(19) United States
(12) Patent Application Publication
(10) Pub. No.: US 2025/0127924 A1
(43) Pub. Date: Apr. 24, 2025

(54) GENE EDITING SYSTEMS COMPRISING AN RNA GUIDE TARGETING STATHMIN 2 (STMN2) AND USES THEREOF

(71) Applicant: Arbor Biotechnologies, Inc., Cambridge, MA (US)

(72) Inventors: Tia Marie DITOMMASO, Newton, MA (US); Anthony James GARRITY, Hingham, MA (US); Noah Michael JAKIMO, Cambridge, MA (US); Quinton Norman WESSELLS, Cambridge, MA (US)

(21) Appl. No.: 18/682,643

(22) PCT Filed: Aug. 11, 2022
(86) PCT No.: PCT/US2022/040042

§ 371 Date: Feb. 9, 2024

Related U.S. Application Data
(60) Provisional application No. 63/322,002, filed on Mar. 21, 2022.
(60) Provisional application No. 63/231,784, filed on Aug. 11, 2021.

(51) Int. Cl. A61K 48/00; A61K 38/17; A61P 25/28; C12N 9/22; C12N 15/11; C12N 15/86

(52) U.S. Cl. CPC A61K 48/005; A61K 38/1709; A61P 25/28; C12N 9/22; C12N 15/111; C12N 15/86; C12N 2310/20; C12N 2750/14143

(57) ABSTRACT

A system for genetic editing of a stathmin 2 (STMN2) gene, comprising (i) a Cas12i2 polypeptide or a first nucleic acid encoding the Cas12i2 polypeptide, and (ii) an RNA guide or second nucleic acid encoding the RNA guide, wherein the RNA guide comprises a spacer sequence specific to a target sequence within an STMN2 gene. Also provided herein are methods for editing a STMN2 gene using the gene editing system disclosed herein and/or for treating diseases associated with the STMN2 gene.

### CROSS REFERENCE TO RELATED APPLICATIONS

This application claims the benefit under 35 U.S.C. § 119 (c) of U.S. Provisional Application No. 63/231,784, filed Aug. 11, 2021, and U.S. Provisional Application No. 63/322,002, filed Mar. 21, 2022, the contents of each of which are incorporated by reference herein in their entirety.

### BACKGROUND

Clustered Regularly Interspaced Short Palindromic Repeats (CRISPR) and CRISPR-associated (Cas) genes, collectively known as CRISPR-Cas or CRISPR/Cas systems, are adaptive immune systems in archaea and bacteria that defend particular species against foreign genetic elements.

### SUMMARY OF THE PRESENT DISCLOSURE

The present disclosure is based, at least in part, on the development of a system for genetic editing of a stathmin 2 (STMN2) gene. The system involves a Cas12i polypeptide such as a Cas12i2 polypeptide and an RNA guide mediating cleavage at a genetic site within the STMN2 gene by the CRISPR nuclease polypeptide. As reported herein, the gene editing system disclosed herein has achieved successful editing of STMN2 gene with high editing efficiency and accuracy.

Without being bound by theory, the gene editing system disclosed herein may exhibit one or more of the following advantageous features. Compared to SpCas9 and Cas12a, Cas12i effectors are smaller (1033 to 1093aa) which, in conjunction with their short mature crRNA (40-43 nt), is preferable in terms of delivery and cost of synthesis. Cas12i cleavage results in larger deletions compared to the small deletions and +1 insertions induced by Cas9 cleavage. Cas12i PAM sequences also differ from those of Cas9. Therefore, larger and different portions of genetic sites of interest can be disrupted with a Cas12i polypeptide and RNA guide compared to Cas9. Using an unbiased approach of tagmentation-based tag integration site sequencing (TTISS), more potential off-target sites with a higher number of unique integration events were identified for SpCas9 compared to Cas12i2. See WO/2021/202800. Therefore, Cas12i such as Cas12i2 may be more specific than Cas9.

Accordingly, provided herein are gene editing systems for editing a STMN2 gene, pharmaceutical compositions or kits comprising such, methods of using the gene editing systems to produce genetically modified cells, and the resultant cells thus produced. Also provided herein are uses of the gene editing systems disclosed herein, the pharmaceutical compositions and kits comprising such, and/or the genetically modified cells thus produced for treating neurodegenerative diseases (e.g., amyotrophic lateral sclerosis (ALS) or frontotemporal dementia (FTD)) in a subject.

In some aspects, the present disclosure features system for genetic editing of a stathmin 2 (STMN2) gene, comprising (i) a Cas12i polypeptide or a first nucleic acid encoding the Cas12i polypeptide, and (ii) an RNA guide or a second nucleic acid encoding the RNA guide. The RNA guide comprises a spacer sequence specific to a target sequence within an STMN2 gene, the target sequence being adjacent to a protospacer adjacent motif (PAM) comprising the motif of 5′-TTN-3′, which is located 5′ to the target sequence.

In some embodiments, the Cas12i is a Cas12i2 polypeptide. In other embodiments, the Cas12i is a Cas12i4 polypeptide.

In some embodiments, the Cas12i polypeptide is a Cas12i2 polypeptide comprising an amino acid sequence at least 95% identical to SEQ ID NO: 448. In some instances, the Cas12i2 polypeptide may comprise one or more mutations relative to SEQ ID NO: 448. In some examples, the one or more mutations in the Cas12i2 polypeptide are at positions D581, G624, F626, P868, I926, V1030, E1035, and/or S1046 of SEQ ID NO: 448. In some examples, the one or more mutations are amino acid substitutions, which optionally is D581R, G624R, F626R, P868T. 1926R. V1030G, E1035R, S1046G, or a combination thereof.

In one example, the Cas12i2 polypeptide comprises mutations at positions D581, D911, 1926, and V1030 (e.g., amino acid substitutions of D581R, D911R, 1926R, and V1030G). In another example, the Cas12i2 polypeptide comprises mutations at positions D581, I926, and V1030 (e.g., amino acid substitutions of D581R, 1926R, and V1030G). In yet another example, the Cas12i2 polypeptide comprises mutations at positions D581, I926, V1030, and S1046 (e.g., amino acid substitutions of D581R, 1926R, V1030G, and S1046G). In still another example, the Cas12i2 polypeptide comprises mutations at positions D581, G624, F626, I926, V1030, E1035, and S1046 (e.g., amino acid substitutions of D581R, G624R, F626R, 1926R, V1030G, E1035R, and S1046G). In another example, the Cas12i2 polypeptide comprises mutations at positions D581, G624, F626, P868, I926, V1030, E1035, and S1046 (e.g., amino acid substitutions of D581R, G624R, F626R, P868T, 1926R, V1030G, E1035R, and S1046G).

Exemplary Cas12i2 polypeptides for use in any of the gene editing systems disclosed herein may comprise the amino acid sequence of any one of SEQ ID NOs: 449-453. In one example, the exemplary Cas12i2 polypeptide for use in any of the gene editing systems disclosed herein comprises the amino acid sequence of SEQ ID NO: 450. In another example, the exemplary Cas12i2 polypeptide for use in any of the gene editing systems disclosed herein comprises the amino acid sequence of SEQ ID NO: 453.

In some embodiments, the gene editing system may comprise the first nucleic acid encoding the Cas12i polypeptide (e.g., the Cas12i2 polypeptide). In some instances, the first nucleic acid is located in a first vector (e.g., a viral vector such as an adeno-associated viral vector or AAV vector). In some instances, the first nucleic acid is a messenger RNA (mRNA). In some instances, the coding sequence for the Cas12i polypeptide is codon optimized.

In some embodiments, the target sequence may be within exon 1, exon 2, exon 3, exon 4, exon 5, exon 6, exon 7, or an intron of the STMN2 gene.

In some embodiments, the RNA guide comprises the sequence of any one of SEQ ID NOs: 4508, 4512, 4559, and 4561, or the second nucleic acid encodes an RNA guide comprising any one of SEQ ID NOs: 4508, 4512, 4559, and 4561.

In some embodiments, the RNA guide comprises the sequence of any one of SEQ ID NOs: 4505, 4506, 4507, 4508, 4509, 4510, 4511, 4512, 4513, 4514, 4515, 4554, 4555, 4556, 4557, 4558, 4559, 4560, 4561, and 4562, or the second nucleic acid encodes an RNA guide comprising any one of SEQ ID NOs: 4505, 4506, 4507, 4508, 4509, 4510, 4511, 4512, 4513, 4514, 4515, 4554, 4555, 4556, 4557, 4558, 4559, 4560, 4561, and 4562.

In some embodiments, the spacer sequence may be 20-30-nucleotides in length. In some examples, the spacer sequence is 20-nucleotides in length.

In some embodiments, the RNA guide comprises the spacer and a direct repeat sequence. In some examples, the direct repeat sequence is 23-36-nucleotides in length. In one example, the direct repeat sequence is at least 90% identical to any one of SEQ ID NOs: 1-10 or a fragment thereof that is at least 23-nucleotides in length. In some specific examples, the direct repeat sequence is any one of SEQ ID NOs: 1-10, or a fragment thereof that is at least 23-nucleotides in length. By way of non-limiting example, the direct repeat sequence is 5′-AGAAAUCCGUCUUUCAUUGACGG-3′ (SEQ ID NO: 10).

In some embodiments, the system may comprise the second nucleic acid encoding the RNA guide. In some examples, the nucleic acid encoding the RNA guide may be located in a viral vector. In some examples, the viral vector comprises the both the first nucleic acid encoding the Cas12i polypeptide (e.g., the Cas12i2 polypeptide) and the second nucleic acid encoding the RNA guide.

In some embodiments, any of the systems described herein may comprise the first nucleic acid encoding the Cas12i polypeptide (e.g., the Cas12i2 polypeptide), which is located in a first vector, and the second nucleic acid encoding the RNA guide, which is located on a second vector. In some examples, the first and/or second vector is a viral vector. In some specific examples, the first and second vectors are the same vector.

In some embodiments, any of the systems described herein may comprise one or more lipid nanoparticles (LNPs), which encompass the Cas12i polypeptide (e.g., the Cas12i2 polypeptide) or the first nucleic acid encoding the Cas12i polypeptide, the RNA guide or the second nucleic acid encoding the RNA guide, or both.

In some embodiments, the system described herein may comprise an LNP, which encompasses the Cas12i polypeptide (e.g., the Cas12i2 polypeptide) or the first nucleic acid encoding the Cas12i polypeptide, and a viral vector comprising the second nucleic acid encoding the RNA guide. In some examples, the viral vector is an AAV vector. In other embodiments, the system described herein may comprise an LNP, which encompasses the RNA guide or the second nucleic acid encoding the RNA guide, and a viral vector comprising the first nucleic acid encoding the Cas12i polypeptide. In some examples, the viral vector is an AAV vector.

In some aspects, the present disclosure also provides a pharmaceutical composition comprising any of the gene editing systems disclosed herein, and a kit comprising the components of the gene editing system.

In other aspects, the present disclosure also features a method for editing a stathmin 2 (STMN2) gene in a cell, the method comprising contacting a host cell with any of the systems disclosed herein to genetically edit the STMN2 gene in the host cell. In some examples, the host cell is cultured in vitro. In other examples, the contacting step is performed by administering the system for editing the STMN2 gene to a subject comprising the host cell.

Also within the scope of the present disclosure is a cell comprising a disrupted a stathmin 2 (STMN2) gene, which can be produced by contacting a host cell with the system disclosed herein genetically edit the STMN2 gene in the host cell.

Still in other aspects, the present disclosure provides a method for treating neurodegenerative diseases (e.g., amyotrophic lateral sclerosis (ALS) or frontotemporal dementia (FTD)) in a subject. The method may comprise administering to a subject in need thereof any of the systems for editing a stathmin 2 (STMN2) gene or any of the cells disclosed herein.

Also provided herein is an RNA guide, comprising (i) a spacer sequence as disclosed herein that is specific to a target sequence in a stathmin 2 (STMN2) gene, wherein the target sequence is adjacent to a protospacer adjacent motif (PAM) comprising the motif of 5′-TTN-3′, which is located 5′ to the target sequence; and (ii) a direct repeat sequence.

In some embodiments, the spacer may be 20-30-nucleotides in length. In some examples, the spacer is 20-nucleotides in length.

In some embodiments, the direct repeat sequence may be 23-36-nucleotides in length. In some examples, the direct repeat sequence is 23-nucleotides in length.

In some embodiments, the target sequence is within exon 1, exon 2, exon 3, exon 4, exon 5, exon 6, exon 7, or an intron of the STMN2 gene.

In some embodiments, the RNA guide comprises the sequence of any one of SEQ ID NOs: 4508, 4512, 4559, and 4561, or the second nucleic acid encodes an RNA guide comprising any one of SEQ ID NOs: 4508, 4512, 4559, and 4561.

In some embodiments, the RNA guide comprises the sequence of any one of SEQ ID NOs: 4505, 4506, 4507, 4508, 4509, 4510, 4511, 4512, 4513, 4514, 4515, 4554, 4555, 4556, 4557, 4558, 4559, 4560, 4561, and 4562, or the second nucleic acid encodes an RNA guide comprising any one of SEQ ID NOs: 4505, 4506, 4507, 4508, 4509, 4510, 4511, 4512, 4513, 4514, 4515, 4554, 4555, 4556, 4557, 4558, 4559, 4560, 4561, and 4562.

In some embodiments, the direct repeat sequence may be at least 90% identical to any one of SEQ ID NOs: 1-10 or a fragment thereof that is at least 23-nucleotides in length. In some examples, the direct repeat sequence is any one of SEQ ID NOs: 1-10, or a fragment thereof that is at least 23-nucleotides in length. By way of non-limiting example, the direct repeat sequence is 5′-AGAAAUCCGUCUUUCAUUGACGG-3′ (SEQ ID NO: 10).

Also provided herein are any of the gene editing systems disclosed herein, pharmaceutical compositions or kits comprising such, or genetically modified cells generated by the gene editing system for use in treating neurodegenerative disease (e.g., amyotrophic lateral sclerosis (ALS) or frontotemporal dementia (FTD)) in a subject, as well as uses of the gene editing systems disclosed herein, pharmaceutical compositions or kits comprising such, or genetically modified cells generated by the gene editing system for manufacturing a medicament for treatment of neurodegenerative disease (e.g., amyotrophic lateral sclerosis (ALS) or frontotemporal dementia (FTD)) in a subject.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 shows editing percentage of STMN2 intron target sequences by the indicated guides, as described in Example 1.

FIG. 2A shows disruption of >15% of the cryptic splice site in STMN2 intron 1 by guides 4, 8, 55, and 57.

FIG. 2B shows disruption of >15% of at least one of 3 TDP-43 binding motifs in STMN2 intron 1 by guides 12, 46, 47, 48, and 49.

FIG. 2C shows disruption of >15% of the premature polyadenylation signal in STMN2 intron 1 by guides 17 and 18.

FIG. 3 is a schematic showing the positions where each of the indicated RNA guides binds intron 1 of STMN2 relative to the positions of the cryptic splice site, the TDP-43 binding motifs, and the premature polyadenylation signal.

FIG. 4 shows indel activity of the tested RNA guides in SH-SY5Y cells.

FIG. 5A is a plot comparing indel activity (% indels) demonstrated in HEK293T cells and SH-SY5Y cells from Example 1 and Example 2, respectively. FIG. 5B is a plot comparing splice site motif disruption demonstrated in HEK293T cells and SH-SY5Y cells from Example 1 and Example 2, respectively.

### DETAILED DESCRIPTION

The present disclosure relates to a system for genetic editing of a stathmin 2 (STMN2) gene, which comprises (i) a Cas12i polypeptide or a first nucleic acid encoding the Cas12i2 polypeptide; and (ii) an RNA guide or a second nucleic acid encoding the RNA guide, wherein the RNA guide comprises a spacer sequence specific to a target sequence within a STMN2 gene, the target sequence being adjacent to a protospacer adjacent motif (PAM) comprising the motif of 5′-TTN-3′, which is located 5′ to the target sequence. Also provided in the present disclosure are a pharmaceutical composition or a kit comprising such a system as well as uses thereof. Further disclosed herein are a method for editing a STMN2 gene in a cell, a cell so produced that comprises a disrupted a STMN2 gene, a method of treating neurodegenerative disease in a subject, and an RNA guide that comprises (i) a spacer sequence that is specific to a target sequence in a STMN2 gene, wherein the target sequence is adjacent to a protospacer adjacent motif (PAM) comprising the motif of 5′-TTN-3′, which is located 5′ to the target sequence; and (ii) a direct repeat sequence, as well as uses thereof.

The Cas12i polypeptide for use in the gene editing system disclosed herein may be a Cas12i2 polypeptide, e.g., a wild-type Cas12i polypeptide or a variant thereof as those disclosed herein. In some examples, the Cas12i2 polypeptide comprises an amino acid sequence at least 95% identical to SEQ ID NO: 448 and comprises one or more mutations relative to SEQ ID NO: 448. In other examples, the Cas12i polypeptide may be a Cas12i4 polypeptide, which is also disclosed herein.

### Definitions

The present disclosure will be described with respect to particular embodiments and with reference to certain Figures, but the present disclosure is not limited thereto but only by the claims. Terms as set forth hereinafter are generally to be understood in their common sense unless indicated otherwise.

As used herein, the term “activity” refers to a biological activity. In some embodiments, activity includes enzymatic activity, e.g., catalytic ability of a Cas12i polypeptide. For example, activity can include nuclease activity.

As used herein the term “STMN2” refers to “stathmin-2.” STMN2 is a neuron-specific member of the stathmin family of proteins and plays roles in regulation of microtubule stability and signal transduction. SEQ ID NO: 454 as set forth herein provides an example of a STMN2 gene sequence. Reference is also made to Gene ID: 11075 for this sequence (www.ncbi.nlm.nih.gov/gene/11075).

As used herein, the term “Cas12i polypeptide” (also referred to herein as Cas12i) refers to a polypeptide that binds to a target sequence on a target nucleic acid specified by an RNA guide, wherein the polypeptide has at least some amino acid sequence homology to a wild-type Cas12i polypeptide. In some embodiments, the Cas12i polypeptide comprises at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with any one of SEQ ID NOs: 1-5 and 11-18 of U.S. Pat. No. 10,808,245, which is incorporated by reference for the subject matter and purpose referenced herein. In some embodiments, a Cas12i polypeptide comprises at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with any one of SEQ ID NOs: 4503, 448, 4504, and 482 of the present application. In some embodiments, a Cas12i polypeptide of the disclosure is a Cas12i2 polypeptide as described in WO/2021/202800, the relevant disclosures of which are incorporated by reference for the subject matter and purpose referenced herein. In some embodiments, the Cas12i polypeptide cleaves a target nucleic acid (e.g., as a nick or a double strand break).

As used herein, the term “adjacent to” refers to a nucleotide or amino acid sequence in close proximity to another nucleotide or amino acid sequence. In some embodiments, a nucleotide sequence is adjacent to another nucleotide sequence if no nucleotides separate the two sequences (i.e., immediately adjacent). In some embodiments, a nucleotide sequence is adjacent to another nucleotide sequence if a small number of nucleotides separate the two sequences (e.g., about 1, 2, 3, 4, 5, 6, 7, 8, 9, 10, 11, 12, 13, 14, 15, 16, 17, 18, 19, or 20 nucleotides). In some embodiments, a first sequence is adjacent to a second sequence if the two sequences are separated by about 5, 6, 7, 8, 9, 10, 11, 12, 13, 14, or 15 nucleotides. In some embodiments, a first sequence is adjacent to a second sequence if the two sequences are separated by up to 2 nucleotides, up to 5 nucleotides, up to 8 nucleotides, up to 10 nucleotides, up to 12 nucleotides, or up to 15 nucleotides. In some embodiments, a first sequence is adjacent to a second sequence if the two sequences are separated by 2-5 nucleotides, 4-6 nucleotides, 4-8 nucleotides, 4-10 nucleotides, 6-8 nucleotides, 6-10 nucleotides, 6-12 nucleotides, 8-10 nucleotides, 8-12 nucleotides, 10-12 nucleotides, 10-15 nucleotides, or 12-15 nucleotides.

As used herein, the term “complex” refers to a grouping of two or more molecules. In some embodiments, the complex comprises a polypeptide and a nucleic acid molecule interacting with (e.g., binding to, coming into contact with, adhering to) one another. For example, the term “complex” can refer to a grouping of an RNA guide and a polypeptide (e.g., a Cas12i polypeptide). Alternatively, the term “complex” can refer to a grouping of an RNA guide, a polypeptide, and the complementary region of a target sequence. As used herein, the term “complex” can refer to a grouping of a STMN2-targeting RNA guide and a Cas12i polypeptide.

As used herein, the term “protospacer adjacent motif” or “PAM” refers to a DNA sequence adjacent to a target sequence (e.g., a STMN2 target sequence). In a double-stranded DNA molecule, the strand containing the PAM motif is called the “PAM-strand” and the complementary strand is called the “non-PAM strand.” The RNA guide binds to a site in the non-PAM strand that is complementary to a target sequence disclosed herein.

In some embodiments, the PAM strand is a coding (e.g., sense) strand. In other embodiments, the PAM strand is a non-coding (e.g., antisense strand). Since an RNA guide binds the non-PAM strand via base-pairing, the non-PAM strand is also known as the target strand, while the PAM strand is also known as the non-target strand.

As used herein, the term “target sequence” refers to a DNA fragment adjacent to a PAM motif (on the PAM strand). The complementary region of the target sequence is on the non-PAM strand. A target sequence may be immediately adjacent to the PAM motif. Alternatively, the target sequence and the PAM may be separately by a small sequence segment (e.g., up to 5 nucleotides, for example, up to 4, 3, 2, or 1 nucleotide). A target sequence may be located at the 3′ end of the PAM motif or at the 5′ end of the PAM motif, depending upon the CRISPR nuclease that recognizes the PAM motif, which is known in the art. For example, a target sequence is located at the 3′ end of a PAM motif for a Cas12i polypeptide (e.g., a Cas12i2 polypeptide such as those disclosed herein). In some embodiments, the target sequence is a sequence within a STMN2 gene sequence, including, but not limited, to the sequence set forth in SEQ ID NO: 454.

As used herein, the term “spacer” or “spacer sequence” is a portion in an RNA guide that is the RNA equivalent of the target sequence (a DNA sequence). The spacer contains a sequence capable of binding to the non-PAM strand via base-pairing at the site complementary to the target sequence (in the PAM strand). Such a spacer is also known as specific to the target sequence. In some instances, the spacer may be at least 75% identical to the target sequence (e.g., at least 80%, at least 85%, at least 90%, at least 95%, at least 98%, or at least 99%), except for the RNA-DNA sequence difference. In some instances, the spacer may be 100% identical to the target sequence except for the RNA-DNA sequence difference.

As used herein, the term “RNA guide” or “RNA guide sequence” refers to any RNA molecule or a modified RNA molecule that facilitates the targeting of a polypeptide (e.g., a Cas12i polypeptide) described herein to a target sequence (e.g., a sequence of a STMN2 gene). For example, an RNA guide can be a molecule that is designed to be complementary to a specific nucleic acid sequence (a target sequence such as a target sequence within a STMN2 gene). An RNA guide may comprise a spacer sequence and a direct repeat (DR) sequence. In some instances, the RNA guide can be a modified RNA molecule comprising one or more deoxyribonucleotides, for example, in a DNA-binding sequence contained in the RNA guide, which binds a sequence complementary to the target sequence. In some examples, the DNA-binding sequence may contain a DNA sequence or a DNA/RNA hybrid sequence. The terms CRISPR RNA (crRNA), pre-crRNA, and mature crRNA are also used herein to refer to an RNA guide.

As used herein, the term “complementary” refers to a first polynucleotide (e.g., a spacer sequence of an RNA guide) that has a certain level of complementarity to a second polynucleotide (e.g., the complementary sequence of a target sequence) such that the first and second polynucleotides can form a double-stranded complex via base-pairing to permit an effector polypeptide that is complexed with the first polynucleotide to act on (e.g., cleave) the second polynucleotide. In some embodiments, the first polynucleotide may be substantially complementary to the second polynucleotide, i.e., having at least about 80%, 81%, 82%, 83%, 84%, 85%, 86%, 87%, 88%, 89%, 90%, 91%, 92%, 93%, 94%, 95%, 96%, 97%, 98%, or 99% complementarity to the second polynucleotide. In some embodiments, the first polynucleotide is completely complementary to the second polynucleotide, i.e., having 100% complementarity to the second polynucleotide.

The “percent identity” (a.k.a., sequence identity) of two nucleic acids or of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST nucleotide searches can be performed with the NBLAST program, score=100, wordlength-12 to obtain nucleotide sequences homologous to the nucleic acid molecules of the present disclosure. BLAST protein searches can be performed with the XBLAST program, score=50, word length=3 to obtain amino acid sequences homologous to the protein molecules of the present disclosure. Where gaps exist between two sequences, Gapped BLAST can be utilized as described in Altschul et al., Nucleic Acids Res. 25 (17): 3389-3402, 1997. When utilizing BLAST and Gapped BLAST programs, the default parameters of the respective programs (e.g., XBLAST and NBLAST) can be used.

As used herein, the term “edit” refers to one or more modifications introduced into a target nucleic acid, e.g., within the STMN2 gene. The edit can be one or more substitutions, one or more insertions, one or more deletions, or a combination thereof. As used herein, the term “substitution” refers to a replacement of a nucleotide or nucleotides with a different nucleotide or nucleotides, relative to a reference sequence. As used herein, the term “insertion” refers to a gain of a nucleotide or nucleotides in a nucleic acid sequence, relative to a reference sequence. As used herein, the term “deletion” refers to a loss of a nucleotide or nucleotides in a nucleic acid sequence, relative to a reference sequence.

No particular process is implied in how to make a sequence comprising a deletion. For instance, a sequence comprising a deletion can be synthesized directly from individual nucleotides. In other embodiments, a deletion is made by providing and then altering a reference sequence. The nucleic acid sequence can be in a genome of an organism. The nucleic acid sequence can be in a cell. The nucleic acid sequence can be a DNA sequence. The deletion can be a frameshift mutation or a non-frameshift mutation. A deletion described herein refers to a deletion of up to several kilobases.

As used herein, the terms “upstream” and “downstream” refer to relative positions within a single nucleic acid (e.g., DNA) sequence in a nucleic acid molecule. “Upstream” and “downstream” relate to the 5′ to 3′ direction, respectively, in which RNA transcription occurs. A first sequence is upstream of a second sequence when the 3′ end of the first sequence occurs before the 5′ end of the second sequence. A first sequence is downstream of a second sequence when the 5′ end of the first sequence occurs after the 3′ end of the second sequence. In some embodiments, the 5′-NTTN-3′ or 5′-TTN-3′ sequence is upstream of an indel described herein, and a Cas12i-induced indel is downstream of the 5′-NTTN-3′ or 5′-TTN-3′ sequence.

### I. Gene Editing Systems

In some aspects, the present disclosure provides gene editing systems comprising an RNA guide targeting a STMN2 gene. Such a gene editing system can be used to edit the STMN2 target gene, e.g., to disrupt the STMN2 gene.

As used herein the term “STMN2” refers to “stathmin-2.” STMN2 is a neuron-specific member of the stathmin family of proteins and plays roles in regulation of microtubule stability and signal transduction. SEQ ID NO: 454 as set forth herein provides an example of a STMN2 gene sequence. Reference is also made to Gene ID: 11075 for this sequence (www.ncbi.nlm.nih.gov/gene/11075).

In some embodiments, the RNA guide is comprised of a direct repeat component and a spacer sequence. In some embodiments, the RNA guide binds a Cas12i polypeptide. In some embodiments, the spacer sequence is specific to a STMN2 target sequence, wherein the STMN2 target sequence is adjacent to a 5′-NTTN-3′ or 5′-TTN-3′ PAM sequence as described herein. In the case of a double-stranded target, the RNA guide binds to a first strand of the target (i.e., the non-PAM strand) and a PAM sequence as described herein is present in the second, complementary strand (i.e., the PAM strand).

In some embodiments, the present disclosure provides compositions comprising a complex, wherein the complex comprises an RNA guide targeting a STMN2. In some embodiments, the present disclosure comprises a complex comprising an RNA guide and a Cas12i polypeptide. In some embodiments, the RNA guide and the Cas12i polypeptide bind to each other in a molar ratio of about 1:1. In some embodiments, a complex comprising an RNA guide and a Cas12i polypeptide binds to the complementary region of a target sequence within a STMN2 gene. In some embodiments, a complex comprising an RNA guide targeting a STMN2 and a Cas12i polypeptide binds to the complementary region of a target sequence within the STMN2 gene at a molar ratio of about 1:1. In some embodiments, the complex comprises enzymatic activity, such as nuclease activity, that can cleave the STMN2 target sequence and/or the complementary sequence. The RNA guide, the Cas12i polypeptide, and the complementary region of the STMN2 target sequence, either alone or together, do not naturally occur. In some embodiments, the RNA guide in the complex comprises a direct repeat and/or a spacer sequence described herein.

In some embodiments, the present disclosure comprises compositions comprising an RNA guide as described herein and/or an RNA encoding a Cas12i polypeptide as described herein. In some embodiments, the RNA guide and the RNA encoding a Cas12i polypeptide are comprised together within the same composition. In some embodiments, the RNA guide and the RNA encoding a Cas12i polypeptide are comprised within separate compositions. In some embodiments, the RNA guide comprises a direct repeat and/or a spacer sequence described herein.

Use of the gene editing systems disclosed herein has advantages over those of other known nuclease systems. Cas12i polypeptides are smaller than other nucleases. For example, Cas12i2 is 1,054 amino acids in length, whereas S. pyogenes Cas9 (SpCas9) is 1.368 amino acids in length, S. thermophilus Cas9 (StCas9) is 1,128 amino acids in length, FnCpf1 is 1,300 amino acids in length, AsCpf1 is 1,307 amino acids in length, and LbCpf1 is 1,246 amino acids in length. Cas12i RNA guides, which do not require a trans-activating CRISPR RNA (tracrRNA), are also smaller than Cas9 RNA guides. The smaller Cas12i polypeptide and RNA guide sizes are beneficial for delivery. Compositions comprising a Cas12i polypeptide also demonstrate decreased off-target activity compared to compositions comprising an SpCas9 polypeptide. See, WO/2021/202800, the relevant disclosures of which are incorporated by reference for the subject matter and purpose referenced herein. Furthermore, indels induced by compositions comprising a Cas12i polypeptide differ from indels induced by compositions comprising an SpCas9 polypeptide. For example, SpCas9 polypeptides primarily induce insertions and deletions of 1 nucleotide in length. However, Cas12i polypeptides induce larger deletions, which can be beneficial in disrupting a larger portion of a gene such as STMN2.

Also provided herein is a system for genetic editing of a STMN2 gene, which comprises (i) a Cas12i polypeptide (e.g., a Cas12i2 polypeptide) or a first nucleic acid encoding the Cas12i polypeptide (e.g., a Cas12i2 polypeptide comprises an amino acid sequence at least 95% identical to SEQ ID NO: 448, which may and comprises one or more mutations relative to SEQ ID NO: 448); and (ii) an RNA guide or a second nucleic acid encoding the RNA guide, wherein the RNA guide comprises a spacer sequence specific to a target sequence within the STMN2 gene (e.g., within exon 1, exon 2, exon 3, exon 4, exon 5, exon 6, exon 7, or an intron of the STMN2 gene), the target sequence being adjacent to a protospacer adjacent motif (PAM) comprising the motif of 5′-TTN-3′ (5′-NTTN-3′), which is located 5′ to the target sequence.

### A. RNA Guides

In some embodiments, the gene editing system described herein comprises an RNA guide targeting a STMN2 gene, for example, targeting exon 1, exon 2, exon 3, exon 4, exon 5, exon 6, exon 7, or an intron of the STMN2 gene. In some embodiments, the gene editing system described herein may comprise two or more (e.g., 2, 3, 4, 5, 6, 7, 8, 9, or more) RNA guides targeting STMN2.

The RNA guide may direct the Cas12i polypeptide contained in the gene editing system as described herein to an STMN2 target sequence. Two or more RNA guides may direct two or more separate Cas12i polypeptides (e.g., Cas12i polypeptides having the same or different sequence) as described herein to two or more (e.g., 2, 3, 4, 5, 6, 7, 8, 9, or more) STMN2 target sequences. Those skilled in the art reading the below examples of particular kinds of RNA guides will understand that, in some embodiments, an RNA guide is STMN2 target-specific. That is, in some embodiments, an RNA guide binds specifically to one or more STMN2 target sequences (e.g., within a cell) and not to non-targeted sequences (e.g., non-specific DNA or random sequences within the same cell).

In some embodiments, the RNA guide comprises a spacer sequence followed by a direct repeat sequence, referring to the sequences in the 5′ to 3′ direction. In some embodiments, the RNA guide comprises a first direct repeat sequence followed by a spacer sequence and a second direct repeat sequence, referring to the sequences in the 5′ to 3′ direction. In some embodiments, the first and second direct repeats of such an RNA guide are identical. In some embodiments, the first and second direct repeats of such an RNA guide are different.

In some embodiments, the spacer sequence and the direct repeat sequence(s) of the RNA guide are present within the same RNA molecule. In some embodiments, the spacer and direct repeat sequences are linked directly to one another. In some embodiments, a short linker is present between the spacer and direct repeat sequences, e.g., an RNA linker of 1, 2, or 3 nucleotides in length. In some embodiments, the spacer sequence and the direct repeat sequence(s) of the RNA guide are present in separate molecules, which are joined to one another by base pairing interactions.

Additional information regarding exemplary direct repeat and spacer components of RNA guides is provided as follows.

### (i). Direct Repeat

In some embodiments, the RNA guide comprises a direct repeat sequence. In some embodiments, the direct repeat sequence of the RNA guide has a length of between 12-100, 13-75, 14-50, or 15-40 nucleotides (e.g., 15, 16, 17, 18, 19, 20, 21, 22, 23, 24, 25, 26, 27, 28, 29, 30, 31, 32, 33, 34, 35, 36, 37, 38, 39, or 40 nucleotides).

In some embodiments, the direct repeat sequence is a sequence of Table 1 or a portion of a sequence of Table 1. The direct repeat sequence can comprise nucleotide 1 through nucleotide 36 of any one of SEQ ID NOs: 1, 2, 3, 4, 5, 6, 7, or 8. The direct repeat sequence can comprise nucleotide 2 through nucleotide 36 of any one of SEQ ID NOs: 1, 2, 3, 4, 5, 6, 7, or 8. The direct repeat sequence can comprise nucleotide 3 through nucleotide 36 of any one of SEQ ID NOs: 1, 2, 3, 4, 5, 6, 7, or 8. The direct repeat sequence can comprise nucleotide 4 through nucleotide 36 of any one of SEQ ID NOs: 1, 2, 3, 4, 5, 6, 7, or 8. The direct repeat sequence can comprise nucleotide 5 through nucleotide 36 of any one of SEQ ID NOs: 1, 2, 3, 4, 5, 6, 7, or 8. The direct repeat sequence can comprise nucleotide 6 through nucleotide 36 of any one of SEQ ID NOs: 1, 2, 3, 4, 5, 6, 7, or 8. The direct repeat sequence can comprise nucleotide 7 through nucleotide 36 of any one of SEQ ID NOs: 1, 2, 3, 4, 5, 6, 7, or 8. The direct repeat sequence can comprise nucleotide 8 through nucleotide 36 of any one of SEQ ID NOs: 1, 2, 3, 4, 5, 6, 7, or 8. The direct repeat sequence can comprise nucleotide 9 through nucleotide 36 of any one of SEQ ID NOs: 1, 2, 3, 4, 5, 6, 7, or 8. The direct repeat sequence can comprise nucleotide 10 through nucleotide 36 of any one of SEQ ID NOs: 1, 2, 3, 4, 5, 6, 7, or 8. The direct repeat sequence can comprise nucleotide 11 through nucleotide 36 of any one of SEQ ID NOs: 1, 2, 3, 4, 5, 6, 7, or 8. The direct repeat sequence can comprise nucleotide 12 through nucleotide 36 of any one of SEQ ID NOs: 1, 2, 3, 4, 5, 6, 7, or 8. The direct repeat sequence can comprise nucleotide 13 through nucleotide 36 of any one of SEQ ID NOs: 1, 2, 3, 4, 5, 6, 7, or 8. The direct repeat sequence can comprise nucleotide 14 through nucleotide 36 of any one of SEQ ID NOs: 1, 2, 3, 4, 5, 6, 7, or 8. The direct repeat sequence can comprise nucleotide 1 through nucleotide 34 of SEQ ID NO: 9. The direct repeat sequence can comprise nucleotide 2 through nucleotide 34 of SEQ ID NO: 9. The direct repeat sequence can comprise nucleotide 3 through nucleotide 34 of SEQ ID NO: 9. The direct repeat sequence can comprise nucleotide 4 through nucleotide 34 of SEQ ID NO: 9. The direct repeat sequence can comprise nucleotide 5 through nucleotide 34 of SEQ ID NO: 9. The direct repeat sequence can comprise nucleotide 6 through nucleotide 34 of SEQ ID NO: 9. The direct repeat sequence can comprise nucleotide 7 through nucleotide 34 of SEQ ID NO: 9. The direct repeat sequence can comprise nucleotide 8 through nucleotide 34 of SEQ ID NO: 9. The direct repeat sequence can comprise nucleotide 9 through nucleotide 34 of SEQ ID NO: 9. The direct repeat sequence can comprise nucleotide 10 through nucleotide 34 of SEQ ID NO: 9. The direct repeat sequence can comprise nucleotide 11 through nucleotide 34 of SEQ ID NO: 9. The direct repeat sequence can comprise nucleotide 12 through nucleotide 34 of SEQ ID NO: 9. In some embodiments, the direct repeat sequence is set forth in SEQ ID NO: 10. In some embodiments, the direct repeat sequence comprises a portion of the sequence set forth in SEQ ID NO: 10.

In some embodiments, the direct repeat sequence has at least 90% identity (e.g., at least 90%, 91%, 92%, 93%, 94%, 95%, 96%, 97%, 98% or 99% identity) to a sequence of Table 1 or a portion of a sequence of Table 1. The direct repeat sequence can have at least 90% identity to a sequence comprising nucleotide 1 through nucleotide 36 of any one of SEQ ID NOs: 1, 2, 3, 4, 5, 6, 7, or 8. The direct repeat sequence can have at least 90% identity to a sequence comprising 2 through nucleotide 36 of any one of SEQ ID NOs: 1, 2, 3, 4, 5, 6, 7, or 8. The direct repeat sequence can have at least 90% identity to a sequence comprising 3 through nucleotide 36 of any one of SEQ ID NOs: 1, 2, 3, 4, 5, 6, 7, or 8. The direct repeat sequence can have at least 90% identity to a sequence comprising 4 through nucleotide 36 of any one of SEQ ID NOs: 1, 2, 3, 4, 5, 6, 7, or 8. The direct repeat sequence can have at least 90% identity to a sequence comprising 5 through nucleotide 36 of any one of SEQ ID NOs: 1, 2, 3, 4, 5, 6, 7, or 8. The direct repeat sequence can have at least 90% identity to a sequence comprising 6 through nucleotide 36 of any one of SEQ ID NOs: 1, 2, 3, 4, 5, 6, 7, or 8. The direct repeat sequence can have at least 90% identity to a sequence comprising 7 through nucleotide 36 of any one of SEQ ID NOs: 1, 2, 3, 4, 5, 6, 7, or 8. The direct repeat sequence can have at least 90% identity to a sequence comprising 8 through nucleotide 36 of any one of SEQ ID NOs: 1, 2, 3, 4, 5, 6, 7, or 8. The direct repeat sequence can have at least 90% identity to a sequence comprising 9 through nucleotide 36 of any one of SEQ ID NOs: 1, 2, 3, 4, 5, 6, 7, or 8. The direct repeat sequence can have at least 90% identity to a sequence comprising 10 through nucleotide 36 of any one of SEQ ID NOs: 1, 2, 3, 4, 5, 6, 7, or 8. The direct repeat sequence can have at least 90% identity to a sequence comprising 11 through nucleotide 36 of any one of SEQ ID NOs: 1, 2, 3, 4, 5, 6, 7, or 8. The direct repeat sequence can have at least 90% identity to a sequence comprising 12 through nucleotide 36 of any one of SEQ ID NOs: 1, 2, 3, 4, 5, 6, 7, or 8. The direct repeat sequence can have at least 90% identity to a sequence comprising 13 through nucleotide 36 of any one of SEQ ID NOs: 1, 2, 3, 4, 5, 6, 7, or 8. The direct repeat sequence can have at least 90% identity to a sequence comprising 14 through nucleotide 36 of any one of SEQ ID NOs: 1, 2, 3, 4, 5, 6, 7, or 8. The direct repeat sequence can have at least 90% identity to a sequence comprising 1 through nucleotide 34 of SEQ ID NO: 9. The direct repeat sequence can have at least 90% identity to a sequence comprising 2 through nucleotide 34 of SEQ ID NO: 9. The direct repeat sequence can have at least 90% identity to a sequence comprising 3 through nucleotide 34 of SEQ ID NO: 9. The direct repeat sequence can have at least 90% identity to a sequence comprising 4 through nucleotide 34 of SEQ ID NO: 9. The direct repeat sequence can have at least 90% identity to a sequence comprising 5 through nucleotide 34 of SEQ ID NO: 9. The direct repeat sequence can have at least 90% identity to a sequence comprising 6 through nucleotide 34 of SEQ ID NO: 9. The direct repeat sequence can have at least 90% identity to a sequence comprising 7 through nucleotide 34 of SEQ ID NO: 9. The direct repeat sequence can have at least 90% identity to a sequence comprising 8 through nucleotide 34 of SEQ ID NO: 9. The direct repeat sequence can have at least 90% identity to a sequence comprising 9 through nucleotide 34 of SEQ ID NO: 9. The direct repeat sequence can have at least 90% identity to a sequence comprising 10 through nucleotide 34 of SEQ ID NO: 9. The direct repeat sequence can have at least 90% identity to a sequence comprising 11 through nucleotide 34 of SEQ ID NO: 9. The direct repeat sequence can have at least 90% identity to a sequence comprising 12 through nucleotide 34 of SEQ ID NO: 9. In some embodiments, the direct repeat sequence has at least 90% identity (e.g., at least 90%, 91%, 92%, 93%, 94%, 95%, 96%, 97%, 98% or 99% identity) to SEQ ID NO: 10. In some embodiments, the direct repeat sequence has at least 90% identity to a portion of the sequence set forth in SEQ ID NO: 10.

In some embodiments, compositions comprising a Cas12i2 polypeptide and an RNA guide comprising the direct repeat of SEQ ID NO: 10 and a spacer length of 20 nucleotides are capable of introducing indels into a STMN2 target sequence.

In some embodiments, the direct repeat sequence is at least 90% identical to the reverse complement of any one of SEQ ID NOs: 1-10 (see, Table 1). In some embodiments, the direct repeat sequence is the reverse complement of any one of SEQ ID NOs: 1-10.

TABLE 1
Cas12i2 Direct Repeat Sequences

| Sequence | |
|---|---|
| identifier | Direct Repeat Sequence |
| SEQ ID NO: 1 | GUUGCAAAACCCAAGAAAUCCGUCUUUCAUUGACGG |
| SEQ ID NO: 2 | AAUAGCGGCCCUAAGAAAUCCGUCUUUCAUUGACGG |
| SEQ ID NO: 3 | AUUGGAACUGGCGAGAAAUCCGUCUUUCAUUGACGG |
| SEQ ID NO: 4 | CCAGCAACACCUAAGAAAUCCGUCUUUCAUUGACGG |
| SEQ ID NO: 5 | CGGCGCUCGAAUAGGAAAUCCGUCUUUCAUUGACGG |
| SEQ ID NO: 6 | GUGGCAACACCUAAGAAAUCCGUCUUUCAUUGACGG |
| SEQ ID NO: 7 | GUUGCAACACCUAAGAAAUCCGUCUUUCAUUGACGG |
| SEQ ID NO: 8 | GUUGCAAUGCCUAAGAAAUCCGUCUUUCAUUGACGG |
| SEQ ID NO: 9 | GCAACACCUAAGAAAUCCGUCUUUCAUUGACGGG |
| SEQ ID NO: 10 | AGAAAUCCGUCUUUCAUUGACGG |

In some embodiments, the direct repeat sequence is a sequence of Table 2 or a portion of a sequence of Table 2. The direct repeat sequence can comprise nucleotide 1 through nucleotide 36 of any one of SEQ ID NOs: 462, 463, 464, 465, 466, 467, 468, 469, 470, 471, 472, 473, 474, 475, 476, 477, 478, or 479. The direct repeat sequence can comprise nucleotide 2 through nucleotide 36 of any one of SEQ ID NOs: 462, 463, 464, 465, 466, 467, 468, 469, 470, 471, 472, 473, 474, 475, 476, 477, 478, or 479. The direct repeat sequence can comprise nucleotide 3 through nucleotide 36 of any one of SEQ ID NOs: 462, 463, 464, 465, 466, 467, 468, 469, 470, 471, 472, 473, 474, 475, 476, 477, 478, or 479. The direct repeat sequence can comprise nucleotide 4 through nucleotide 36 of any one of SEQ ID NOs: 462, 463, 464, 465, 466, 467, 468, 469, 470, 471, 472, 473, 474, 475, 476, 477, 478, or 479. The direct repeat sequence can comprise nucleotide 5 through nucleotide 36 of any one of SEQ ID NOs: 462, 463, 464, 465, 466, 467, 468, 469, 470, 471, 472, 473, 474, 475, 476, 477, 478, or 479. The direct repeat sequence can comprise nucleotide 6 through nucleotide 36 of any one of SEQ ID NOs: 462, 463, 464, 465, 466, 467, 468, 469, 470, 471, 472, 473, 474, 475, 476, 477, 478, or 479. The direct repeat sequence can comprise nucleotide 7 through nucleotide 36 of any one of SEQ ID NOS: 462, 463, 464, 465, 466, 467, 468, 469, 470, 471, 472, 473, 474, 475, 476, 477, 478, or 479. The direct repeat sequence can comprise nucleotide 8 through nucleotide 36 of any one of SEQ ID NOs: 462, 463, 464, 465, 466, 467, 468, 469, 470, 471, 472, 473, 474, 475, 476, 477, 478, or 479. The direct repeat sequence can comprise nucleotide 9 through nucleotide 36 of any one of SEQ ID NOs: 462, 463, 464, 465, 466, 467, 468, 469, 470, 471, 472, 473, 474, 475, 476, 477, 478, or 479. The direct repeat sequence can comprise nucleotide 10 through nucleotide 36 of any one of SEQ ID NOs: 462, 463, 464, 465, 466, 467, 468, 469, 470, 471, 472, 473, 474, 475, 476, 477, 478, or 479. The direct repeat sequence can comprise nucleotide 11 through nucleotide 36 of any one of SEQ ID NOs: 462, 463, 464, 465, 466, 467, 468, 469, 470, 471, 472, 473, 474, 475, 476, 477, 478, or 479. The direct repeat sequence can comprise nucleotide 12 through nucleotide 36 of any one of SEQ ID NOs: 462, 463, 464, 465, 466, 467, 468, 469, 470, 471, 472, 473, 474, 475, 476, 477, 478, or 479. The direct repeat sequence can comprise nucleotide 13 through nucleotide 36 of any one of SEQ ID NOs: 462, 463, 464, 465, 466, 467, 468, 469, 470, 471, 472, 473, 474, 475, 476, 477, 478, or 479. The direct repeat sequence can comprise nucleotide 14 through nucleotide 36 of any one of SEQ ID NOs: 462, 463, 464, 465, 466, 467, 468, 469, 470, 471, 472, 473, 474, 475, 476, 477, 478, or 479.

In some embodiments, the direct repeat sequence has at least 95% identity (e.g., at least 95%, 96%, 97%, 98% or 99% identity) to a sequence of Table 2 or a portion of a sequence of Table 2. The direct repeat sequence can have at least 95% identity to a sequence comprising nucleotide 1 through nucleotide 36 of any one of SEQ ID NOs: 462, 463, 464, 465, 466, 467, 468, 469, 470, 471, 472, 473, 474, 475, 476, 477, 478, or 479. The direct repeat sequence can have at least 95% identity to a sequence comprising 2 through nucleotide 36 of any one of SEQ ID NOs: 462, 463, 464, 465, 466, 467, 468, 469, 470, 471, 472, 473, 474, 475, 476, 477, 478, or 479. The direct repeat sequence can have at least 95% identity to a sequence comprising 3 through nucleotide 36 of any one of SEQ ID NOs: 462, 463, 464, 465, 466, 467, 468, 469, 470, 471, 472, 473, 474, 475, 476, 477, 478, or 479. The direct repeat sequence can have at least 95% identity to a sequence comprising 4 through nucleotide 36 of any one of SEQ ID NOs: 462, 463, 464, 465, 466, 467, 468, 469, 470, 471, 472, 473, 474, 475, 476, 477, 478, or 479. The direct repeat sequence can have at least 95% identity to a sequence comprising 5 through nucleotide 36 of any one of SEQ ID NOs: 462, 463, 464, 465, 466, 467, 468, 469, 470, 471, 472, 473, 474, 475, 476, 477, 478, or 479. The direct repeat sequence can have at least 95% identity to a sequence comprising 6 through nucleotide 36 of any one of SEQ ID NOs: 462, 463, 464, 465, 466, 467, 468, 469, 470, 471, 472, 473, 474, 475, 476, 477, 478, or 479. The direct repeat sequence can have at least 95% identity to a sequence comprising 7 through nucleotide 36 of any one of SEQ ID NOs: 462, 463, 464, 465, 466, 467, 468, 469, 470, 471, 472, 473, 474, 475, 476, 477, 478, or 479. The direct repeat sequence can have at least 95% identity to a sequence comprising 8 through nucleotide 36 of any one of SEQ ID NOs: 462, 463, 464, 465, 466, 467, 468, 469, 470, 471, 472, 473, 474, 475, 476, 477, 478, or 479. The direct repeat sequence can have at least 95% identity to a sequence comprising 9 through nucleotide 36 of any one of SEQ ID NOs: 462, 463, 464, 465, 466, 467, 468, 469, 470, 471, 472, 473, 474, 475, 476, 477, 478, or 479. The direct repeat sequence can have at least 95% identity to a sequence comprising 10 through nucleotide 36 of any one of SEQ ID NOs: 462, 463, 464, 465, 466, 467, 468, 469, 470, 471, 472, 473, 474, 475, 476, 477, 478, or 479. The direct repeat sequence can have at least 95% identity to a sequence comprising 11 through nucleotide 36 of any one of SEQ ID NOs: 462, 463, 464, 465, 466, 467, 468, 469, 470, 471, 472, 473, 474, 475, 476, 477, 478, or 479. The direct repeat sequence can have at least 95% identity to a sequence comprising 12 through nucleotide 36 of any one of SEQ ID NOs: 462, 463, 464, 465, 466, 467, 468, 469, 470, 471, 472, 473, 474, 475, 476, 477, 478, or 479. The direct repeat sequence can have at least 95% identity to a sequence comprising 13 through nucleotide 36 of any one of SEQ ID NOs: 462, 463, 464, 465, 466, 467, 468, 469, 470, 471, 472, 473, 474, 475, 476, 477, 478, or 479.

In some embodiments, the direct repeat sequence has at least 90% identity (e.g., at least 90%, 91%, 92%, 93%, 94%, 95%, 96%, 97%, 98% or 99% identity) to a sequence of Table 2 or a portion of a sequence of Table 2. The direct repeat sequence can have at least 90% identity to a sequence comprising nucleotide 1 through nucleotide 36 of any one of SEQ ID NOs: 462, 463, 464, 465, 466, 467, 468, 469, 470, 471, 472, 473, 474, 475, 476, 477, 478, or 479. The direct repeat sequence can have at least 90% identity to a sequence comprising 2 through nucleotide 36 of any one of SEQ ID NOs: 462, 463, 464, 465, 466, 467, 468, 469, 470, 471, 472, 473, 474, 475, 476, 477, 478, or 479. The direct repeat sequence can have at least 90% identity to a sequence comprising 3 through nucleotide 36 of any one of SEQ ID NOs: 462, 463, 464, 465, 466, 467, 468, 469, 470, 471, 472, 473, 474, 475, 476, 477, 478, or 479. The direct repeat sequence can have at least 90% identity to a sequence comprising 4 through nucleotide 36 of any one of SEQ ID NOs: 462, 463, 464, 465, 466, 467, 468, 469, 470, 471, 472, 473, 474, 475, 476, 477, 478, or 479. The direct repeat sequence can have at least 90% identity to a sequence comprising 5 through nucleotide 36 of any one of SEQ ID NOs: 462, 463, 464, 465, 466, 467, 468, 469, 470, 471, 472, 473, 474, 475, 476, 477, 478, or 479. The direct repeat sequence can have at least 90% identity to a sequence comprising 6 through nucleotide 36 of any one of SEQ ID NOs: 462, 463, 464, 465, 466, 467, 468, 469, 470, 471, 472, 473, 474, 475, 476, 477, 478, or 479. The direct repeat sequence can have at least 90% identity to a sequence comprising 7 through nucleotide 36 of any one of SEQ ID NOs: 462, 463, 464, 465, 466, 467, 468, 469, 470, 471, 472, 473, 474, 475, 476, 477, 478, or 479. The direct repeat sequence can have at least 90% identity to a sequence comprising 8 through nucleotide 36 of any one of SEQ ID NOs: 462, 463, 464, 465, 466, 467, 468, 469, 470, 471, 472, 473, 474, 475, 476, 477, 478, or 479. The direct repeat sequence can have at least 90% identity to a sequence comprising 9 through nucleotide 36 of any one of SEQ ID NOs: 462, 463, 464, 465, 466, 467, 468, 469, 470, 471, 472, 473, 474, 475, 476, 477, 478, or 479. The direct repeat sequence can have at least 90% identity to a sequence comprising 10 through nucleotide 36 of any one of SEQ ID NOs: 462, 463, 464, 465, 466, 467, 468, 469, 470, 471, 472, 473, 474, 475, 476, 477, 478, or 479. The direct repeat sequence can have at least 90% identity to a sequence comprising 11 through nucleotide 36 of any one of SEQ ID NOs: 462, 463, 464, 465, 466, 467, 468, 469, 470, 471, 472, 473, 474, 475, 476, 477, 478, or 479. The direct repeat sequence can have at least 90% identity to a sequence comprising 12 through nucleotide 36 of any one of SEQ ID NOs: 462, 463, 464, 465, 466, 467, 468, 469, 470, 471, 472, 473, 474, 475, 476, 477, 478, or 479. The direct repeat sequence can have at least 90% identity to a sequence comprising 13 through nucleotide 36 of any one of SEQ ID NOs: 462, 463, 464, 465, 466, 467, 468, 469, 470, 471, 472, 473, 474, 475, 476, 477, 478, or 479.

In some embodiments, the direct repeat sequence is at least 90% identical to the reverse complement of any one of SEQ ID NOs: 462, 463, 464, 465, 466, 467, 468, 469, 470, 471, 472, 473, 474, 475, 476, 477, 478, or 479. In some embodiments, the direct repeat sequence is at least 95% identical to the reverse complement of any one of SEQ ID NOs: 462, 463, 464, 465, 466, 467, 468, 469, 470, 471, 472, 473, 474, 475, 476, 477, 478, or 479. In some embodiments, the direct repeat sequence is the reverse complement of any one of SEQ ID NOs: 462, 463, 464, 465, 466, 467, 468, 469, 470, 471, 472, 473, 474, 475, 476, 477, 478, or 479.

In some embodiments, the direct repeat sequence is at least 90% identical to SEQ ID NO: 480 or a portion of SEQ ID NO: 480. In some embodiments, the direct repeat sequence is at least 95% identical to SEQ ID NO: 480 or a portion of SEQ ID NO: 480. In some embodiments, the direct repeat sequence is 100% identical to SEQ ID NO: 480 or a portion of SEQ ID NO: 480.

TABLE 2
Cas12i4 Direct Repeat Sequences

| Sequence identifier | Direct Repeat Sequence |
|---|---|
| SEQ ID NO: 462 | UCUCAACGAUAGUCAGACAUGUGUCCUCAGUGACAC |
| SEQ ID NO: 463 | UUUUAACAACACUCAGGCAUGUGUCCACAGUGACAC |
| SEQ ID NO: 464 | UUGAACGGAUACUCAGACAUGUGUUUCCAGUGACAC |
| SEQ ID NO: 465 | UGCCCUCAAUAGUCAGAUGUGUGUCCACAGUGACAC |
| SEQ ID NO: 466 | UCUCAAUGAUACUUAGAUACGUGUCCUCAGUGACAC |
| SEQ ID NO: 467 | UCUCAAUGAUACUCAGACAUGUGUCCCCAGUGACAC |
| SEQ ID NO: 468 | UCUCAAUGAUACUAAGACAUGUGUCCUCAGUGACAC |
| SEQ ID NO: 469 | UCUCAACUAUACUCAGACAUGUGUCCUCAGUGACAC |
| SEQ ID NO: 470 | UCUCAACGAUACUCAGACAUGUGUCCUCAGUGACAC |
| SEQ ID NO: 471 | UCUCAACGAUACUAAGAUAUGUGUCCUCAGCGACAC |
| SEQ ID NO: 472 | UCUCAACGAUACUAAGAUAUGUGUCCCCAGUGACAC |
| SEQ ID NO: 473 | UCUCAACGAUACUAAGAUAUGUGUCCACAGUGACAC |
| SEQ ID NO: 474 | UCUCAACAAUACUCAGACAUGUGUCCCCAGUGACAC |
| SEQ ID NO: 475 | UCUCAACAAUACUAAGGCAUGUGUCCCCAGUGACCC |
| SEQ ID NO: 476 | UCUCAAAGAUACUCAGACACGUGUCCCCAGUGACAC |
| SEQ ID NO: 477 | UCUCAAAAAUACUCAGACAUGUGUCCUCAGUGACAC |
| SEQ ID NO: 478 | GCGAAACAACAGUCAGACAUGUGUCCCCAGUGACAC |
| SEQ ID NO: 479 | CCUCAACGAUAUUAAGACAUGUGUCCGCAGUGACAC |
| SEQ ID NO: 480 | AGACAUGUGUCCUCAGUGACAC |

In some embodiments, the direct repeat sequence is a sequence of Table 3 or a portion of a sequence of Table 3. In some embodiments, the direct repeat sequence has at least 95% identity (e.g., at least 95%, 96%, 97%, 98% or 99% identity) to a sequence of Table 3 or a portion of a sequence of Table 3. In some embodiments, the direct repeat sequence has at least 90% identity (e.g., at least 90%, 91%, 92%, 93%, 94%, 95%, 96%, 97%, 98% or 99% identity) to a sequence of Table 3 or a portion of a sequence of Table 3. In some embodiments, the direct repeat sequence is at least 90% identical to the reverse complement of any one of SEQ ID NOs: 485-487. In some embodiments, the direct repeat sequence is at least 95% identical to the reverse complement of any one of SEQ ID NOs: 485-487. In some embodiments, the direct repeat sequence is the reverse complement of any one of SEQ ID NOs: 485-487.

TABLE 3
Cas12i1 Direct Repeat Sequences

| Sequence identifier | Direct Repeat Sequence |
|---|---|
| SEQ ID NO: 485 | GUUGGAAUGACUAAUUUUUGUGCCCACCGUUGGCAC |
| SEQ ID NO: 486 | AAUUUUUGUGCCCAUCGUUGGCAC |
| SEQ ID NO: 487 | AUUUUUGUGCCCAUCGUUGGCAC |

In some embodiments, the direct repeat sequence is a sequence of Table 4 or a portion of a sequence of Table 4. In some embodiments, the direct repeat sequence has at least 95% identity (e.g., at least 95%, 96%, 97%, 98% or 99% identity) to a sequence of Table 4 or a portion of a sequence of Table 4. In some embodiments, the direct repeat sequence has at least 90% identity (e.g., at least 90%, 91%, 92%, 93%, 94%, 95%, 96%, 97%, 98% or 99% identity) to a sequence of Table 4 or a portion of a sequence of Table 4. In some embodiments, the direct repeat sequence is at least 90% identical to the reverse complement of any one of SEQ ID NOs: 488-490. In some embodiments, the direct repeat sequence is at least 95% identical to the reverse complement of any one of SEQ ID NOs: 488-490. In some embodiments, the direct repeat sequence is the reverse complement of any one of SEQ ID NOs: 488-490.

TABLE 4
Cas12i3 Direct Repeat Sequences.

| Sequence identifier | Direct Repeat Sequence |
|---|---|
| SEQ ID NO: 488 | CUAGCAAUGACCUAAUAGUGUGUCCUUAGUUGACAU |
| SEQ ID NO: 489 | CCUACAAUACCUAAGAAAUCCGUCCUAAGUUGACGG |
| SEQ ID NO: 490 | AUAGUGUGUCCUUAGUUGACAU |

In some embodiments, a direct repeat sequence described herein comprises a uracil (U). In some embodiments, a direct repeat sequence described herein comprises a thymine (T). In some embodiments, a direct repeat sequence according to Tables 1˜4 comprises a sequence comprising a thymine in one or more places indicated as uracil in Tables 1-4.

### (ii). Spacer Sequences

In some embodiments, the RNA guide comprises a DNA targeting or spacer sequence. In some embodiments, the spacer sequence of the RNA guide has a length of between 12-100, 13-75, 14-50, or 15-30 nucleotides (e.g., 15, 16, 17, 18, 19, 20, 21, 22, 23, 24, 25, 26, 27, 28, 29, or 30 nucleotides) and is complementary to a non-PAM strand sequence. In some embodiments, the spacer sequence is designed to be complementary to a specific DNA strand, e.g., of a genomic locus.

In some embodiments, the RNA guide spacer sequence is substantially identical to a complementary strand of a target sequence. In some embodiments, the RNA guide comprises a sequence having at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to a complementary strand of a reference nucleic acid sequence, e.g., target sequence. The percent identity between two such nucleic acids can be determined manually by inspection of the two optimally aligned nucleic acid sequences or by using software programs or algorithms (e.g., BLAST. ALIGN, CLUSTAL) using standard parameters.

In some embodiments, the RNA guide comprises a spacer sequence that has a length of between 12-100, 13-75, 14-50, or 15-30 nucleotides (e.g., 15, 16, 17, 18, 19, 20, 21, 22, 23, 24, 25, 26, 27, 28, 29, or 30 nucleotides) and at least 80%, at least 90%, at least 95%, at least 96%, at least 97%, at least 98%, at least 99% complementary to a region on the non-PAM strand that is complementary to the target sequence. In some embodiments, the RNA guide comprises a sequence at least 80%, at least 90%, at least 95%, at least 96%, at least 97%, at least 98%, at least 99% complementary to a target DNA sequence. In some embodiments, the RNA guide comprises a sequence at least 80%, at least 90%, at least 95%, at least 96%, at least 97%, at least 98%, at least 99% complementary to a target genomic sequence. In some embodiments, the RNA guide comprises a sequence, e.g., RNA sequence, that is a length of up to 50 and at least 80%, at least 90%, at least 95%, at least 96%, at least 97%, at least 98%, at least 99% complementary to a region on the non-PAM strand that is complementary to the target sequence. In some embodiments, the RNA guide comprises a sequence at least 80%, at least 90%, at least 95%, at least 96%, at least 97%, at least 98%, at least 99% complementary to a target DNA sequence. In some embodiments, the RNA guide comprises a sequence at least 80%, at least 90%, at least 95%, at least 96%, at least 97%, at least 98%, at least 99% complementary to a target genomic sequence.

In some embodiments, the spacer sequence is a sequence of Table 5A or 5B or a portion of a sequence of Table 5A or 5B. It should be understood that an indication of SEQ ID NOs: 229-446 or 2497-4502 should be considered as equivalent to a listing of SEQ ID NOs: 229-446 or 2497-4502, with each of the intervening numbers present in the listing, i.e., 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, and 446, or 2497, 2498, 2499, 2500, 2501, 2502, 2503, 2504, 2505, 2506, 2507, 2508, 2509, 2510, 2511, 2512, 2513, 2514, 2515, 2516, 2517, 2518, 2519, 2520, 2521, 2522, 2523, 2524, 2525, 2526, 2527, 2528, 2529, 2530, 2531, 2532, 2533, 2534, 2535, 2536, 2537, 2538, 2539, 2540, 2541, 2542, 2543, 2544, 2545, 2546, 2547, 2548, 2549, 2550, 2551, 2552, 2553, 2554, 2555, 2556, 2557, 2558, 2559, 2560, 2561, 2562, 2563, 2564, 2565, 2566, 2567, 2568, 2569, 2570, 2571, 2572, 2573, 2574, 2575, 2576, 2577, 2578, 2579, 2580, 2581, 2582, 2583, 2584, 2585, 2586, 2587, 2588, 2589, 2590, 2591, 2592, 2593, 2594, 2595, 2596, 2597, 2598, 2599, 2600, 2601, 2602, 2603, 2604, 2605, 2606, 2607, 2608, 2609, 2610, 2611, 2612, 2613, 2614, 2615, 2616, 2617, 2618, 2619, 2620, 2621, 2622, 2623, 2624, 2625, 2626, 2627, 2628, 2629, 2630, 2631, 2632, 2633, 2634, 2635, 2636, 2637, 2638, 2639, 2640, 2641, 2642, 2643, 2644, 2645, 2646, 2647, 2648, 2649, 2650, 2651, 2652, 2653, 2654, 2655, 2656, 2657, 2658, 2659, 2660, 2661, 2662, 2663, 2664, 2665, 2666, 2667, 2668, 2669, 2670, 2671, 2672, 2673, 2674, 2675, 2676, 2677, 2678, 2679, 2680, 2681, 2682, 2683, 2684, 2685, 2686, 2687, 2688, 2689, 2690, 2691, 2692, 2693, 2694, 2695, 2696, 2697, 2698, 2699, 2700, 2701, 2702, 2703, 2704, 2705, 2706, 2707, 2708, 2709, 2710, 2711, 2712, 2713, 2714, 2715, 2716, 2717, 2718, 2719, 2720, 2721, 2722, 2723, 2724, 2725, 2726, 2727, 2728, 2729, 2730, 2731, 2732, 2733, 2734, 2735, 2736, 2737, 2738, 2739, 2740, 2741, 2742, 2743, 2744, 2745, 2746, 2747, 2748, 2749, 2750, 2751, 2752, 2753, 2754, 2755, 2756, 2757, 2758, 2759, 2760, 2761, 2762, 2763, 2764, 2765, 2766, 2767, 2768, 2769, 2770, 2771, 2772, 2773, 2774, 2775, 2776, 2777, 2778, 2779, 2780, 2781, 2782, 2783, 2784, 2785, 2786, 2787, 2788, 2789, 2790, 2791, 2792, 2793, 2794, 2795, 2796, 2797, 2798, 2799, 2800, 2801, 2802, 2803, 2804, 2805, 2806, 2807, 2808, 2809, 2810, 2811, 2812, 2813, 2814, 2815, 2816, 2817, 2818, 2819, 2820, 2821, 2822, 2823, 2824, 2825, 2826, 2827, 2828, 2829, 2830, 2831, 2832, 2833, 2834, 2835, 2836, 2837, 2838, 2839, 2840, 2841, 2842, 2843, 2844, 2845, 2846, 2847, 2848, 2849, 2850, 2851, 2852, 2853, 2854, 2855, 2856, 2857, 2858, 2859, 2860, 2861, 2862, 2863, 2864, 2865, 2866, 2867, 2868, 2869, 2870, 2871, 2872, 2873, 2874, 2875, 2876, 2877, 2878, 2879, 2880, 2881, 2882, 2883, 2884, 2885, 2886, 2887, 2888, 2889, 2890, 2891, 2892, 2893, 2894, 2895, 2896, 2897, 2898, 2899, 2900, 2901, 2902, 2903, 2904, 2905, 2906, 2907, 2908, 2909, 2910, 2911, 2912, 2913, 2914, 2915, 2916, 2917, 2918, 2919, 2920, 2921, 2922, 2923, 2924, 2925, 2926, 2927, 2928, 2929, 2930, 2931, 2932, 2933, 2934, 2935, 2936, 2937, 2938, 2939, 2940, 2941, 2942, 2943, 2944, 2945, 2946, 2947, 2948, 2949, 2950, 2951, 2952, 2953, 2954, 2955, 2956, 2957, 2958, 2959, 2960, 2961, 2962, 2963, 2964, 2965, 2966, 2967, 2968, 2969, 2970, 2971, 2972, 2973, 2974, 2975, 2976, 2977, 2978, 2979, 2980, 2981, 2982, 2983, 2984, 2985, 2986, 2987, 2988, 2989, 2990, 2991, 2992, 2993, 2994, 2995, 2996, 2997, 2998, 2999, 3000, 3001, 3002, 3003, 3004, 3005, 3006, 3007, 3008, 3009, 3010, 3011, 3012, 3013, 3014, 3015, 3016, 3017, 3018, 3019, 3020, 3021, 3022, 3023, 3024, 3025, 3026, 3027, 3028, 3029, 3030, 3031, 3032, 3033, 3034, 3035, 3036, 3037, 3038, 3039, 3040, 3041, 3042, 3043, 3044, 3045, 3046, 3047, 3048, 3049, 3050, 3051, 3052, 3053, 3054, 3055, 3056, 3057, 3058, 3059, 3060, 3061, 3062, 3063, 3064, 3065, 3066, 3067, 3068, 3069, 3070, 3071, 3072, 3073, 3074, 3075, 3076, 3077, 3078, 3079, 3080, 3081, 3082, 3083, 3084, 3085, 3086, 3087, 3088, 3089, 3090, 3091, 3092, 3093, 3094, 3095, 3096, 3097, 3098, 3099, 3100, 3101, 3102, 3103, 3104, 3105, 3106, 3107, 3108, 3109, 3110, 3111, 3112, 3113, 3114, 3115, 3116, 3117, 3118, 3119, 3120, 3121, 3122, 3123, 3124, 3125, 3126, 3127, 3128, 3129, 3130, 3131, 3132, 3133, 3134, 3135, 3136, 3137, 3138, 3139, 3140, 3141, 3142, 3143, 3144, 3145, 3146, 3147, 3148, 3149, 3150, 3151, 3152, 3153, 3154, 3155, 3156, 3157, 3158, 3159, 3160, 3161, 3162, 3163, 3164, 3165, 3166, 3167, 3168, 3169, 3170, 3171, 3172, 3173, 3174, 3175, 3176, 3177, 3178, 3179, 3180, 3181, 3182, 3183, 3184, 3185, 3186, 3187, 3188, 3189, 3190, 3191, 3192, 3193, 3194, 3195, 3196, 3197, 3198, 3199, 3200, 3201, 3202, 3203, 3204, 3205, 3206, 3207, 3208, 3209, 3210, 3211, 3212, 3213, 3214, 3215, 3216, 3217, 3218, 3219, 3220, 3221, 3222, 3223, 3224, 3225, 3226, 3227, 3228, 3229, 3230, 3231, 3232, 3233, 3234, 3235, 3236, 3237, 3238, 3239, 3240, 3241, 3242, 3243, 3244, 3245, 3246, 3247, 3248, 3249, 3250, 3251, 3252, 3253, 3254, 3255, 3256, 3257, 3258, 3259, 3260, 3261, 3262, 3263, 3264, 3265, 3266, 3267, 3268, 3269, 3270, 3271, 3272, 3273, 3274, 3275, 3276, 3277, 3278, 3279, 3280, 3281, 3282, 3283, 3284, 3285, 3286, 3287, 3288, 3289, 3290, 3291, 3292, 3293, 3294, 3295, 3296, 3297, 3298, 3299, 3300, 3301, 3302, 3303, 3304, 3305, 3306, 3307, 3308, 3309, 3310, 3311, 3312, 3313, 3314, 3315, 3316, 3317, 3318, 3319, 3320, 3321, 3322, 3323, 3324, 3325, 3326, 3327, 3328, 3329, 3330, 3331, 3332, 3333, 3334, 3335, 3336, 3337, 3338, 3339, 3340, 3341, 3342, 3343, 3344, 3345, 3346, 3347, 3348, 3349, 3350, 3351, 3352, 3353, 3354, 3355, 3356, 3357, 3358, 3359, 3360, 3361, 3362, 3363, 3364, 3365, 3366, 3367, 3368, 3369, 3370, 3371, 3372, 3373, 3374, 3375, 3376, 3377, 3378, 3379, 3380, 3381, 3382, 3383, 3384, 3385, 3386, 3387, 3388, 3389, 3390, 3391, 3392, 3393, 3394, 3395, 3396, 3397, 3398, 3399, 3400, 3401, 3402, 3403, 3404, 3405, 3406, 3407, 3408, 3409, 3410, 3411, 3412, 3413, 3414, 3415, 3416, 3417, 3418, 3419, 3420, 3421, 3422, 3423, 3424, 3425, 3426, 3427, 3428, 3429, 3430, 3431, 3432, 3433, 3434, 3435, 3436, 3437, 3438, 3439, 3440, 3441, 3442, 3443, 3444, 3445, 3446, 3447, 3448, 3449, 3450, 3451, 3452, 3453, 3454, 3455, 3456, 3457, 3458, 3459, 3460, 3461, 3462, 3463, 3464, 3465, 3466, 3467, 3468, 3469, 3470, 3471, 3472, 3473, 3474, 3475, 3476, 3477, 3478, 3479, 3480, 3481, 3482, 3483, 3484, 3485, 3486, 3487, 3488, 3489, 3490, 3491, 3492, 3493, 3494, 3495, 3496, 3497, 3498, 3499, 3500, 3501, 3502, 3503, 3504, 3505, 3506, 3507, 3508, 3509, 3510, 3511, 3512, 3513, 3514, 3515, 3516, 3517, 3518, 3519, 3520, 3521, 3522, 3523, 3524, 3525, 3526, 3527, 3528, 3529, 3530, 3531, 3532, 3533, 3534, 3535, 3536, 3537, 3538, 3539, 3540, 3541, 3542, 3543, 3544, 3545, 3546, 3547, 3548, 3549, 3550, 3551, 3552, 3553, 3554, 3555, 3556, 3557, 3558, 3559, 3560, 3561, 3562, 3563, 3564, 3565, 3566, 3567, 3568, 3569, 3570, 3571, 3572, 3573, 3574, 3575, 3576, 3577, 3578, 3579, 3580, 3581, 3582, 3583, 3584, 3585, 3586, 3587, 3588, 3589, 3590, 3591, 3592, 3593, 3594, 3595, 3596, 3597, 3598, 3599, 3600, 3601, 3602, 3603, 3604, 3605, 3606, 3607, 3608, 3609, 3610, 3611, 3612, 3613, 3614, 3615, 3616, 3617, 3618, 3619, 3620, 3621, 3622, 3623, 3624, 3625, 3626, 3627, 3628, 3629, 3630, 3631, 3632, 3633, 3634, 3635, 3636, 3637, 3638, 3639, 3640, 3641, 3642, 3643, 3644, 3645, 3646, 3647, 3648, 3649, 3650, 3651, 3652, 3653, 3654, 3655, 3656, 3657, 3658, 3659, 3660, 3661, 3662, 3663, 3664, 3665, 3666, 3667, 3668, 3669, 3670, 3671, 3672, 3673, 3674, 3675, 3676, 3677, 3678, 3679, 3680, 3681, 3682, 3683, 3684, 3685, 3686, 3687, 3688, 3689, 3690, 3691, 3692, 3693, 3694, 3695, 3696, 3697, 3698, 3699, 3700, 3701, 3702, 3703, 3704, 3705, 3706, 3707, 3708, 3709, 3710, 3711, 3712, 3713, 3714, 3715, 3716, 3717, 3718, 3719, 3720, 3721, 3722, 3723, 3724, 3725, 3726, 3727, 3728, 3729, 3730, 3731, 3732, 3733, 3734, 3735, 3736, 3737, 3738, 3739, 3740, 3741, 3742, 3743, 3744, 3745, 3746, 3747, 3748, 3749, 3750, 3751, 3752, 3753, 3754, 3755, 3756, 3757, 3758, 3759, 3760, 3761, 3762, 3763, 3764, 3765, 3766, 3767, 3768, 3769, 3770, 3771, 3772, 3773, 3774, 3775, 3776, 3777, 3778, 3779, 3780, 3781, 3782, 3783, 3784, 3785, 3786, 3787, 3788, 3789, 3790, 3791, 3792, 3793, 3794, 3795, 3796, 3797, 3798, 3799, 3800, 3801, 3802, 3803, 3804, 3805, 3806, 3807, 3808, 3809, 3810, 3811, 3812, 3813, 3814, 3815, 3816, 3817, 3818, 3819, 3820, 3821, 3822, 3823, 3824, 3825, 3826, 3827, 3828, 3829, 3830, 3831, 3832, 3833, 3834, 3835, 3836, 3837, 3838, 3839, 3840, 3841, 3842, 3843, 3844, 3845, 3846, 3847, 3848, 3849, 3850, 3851, 3852, 3853, 3854, 3855, 3856, 3857, 3858, 3859, 3860, 3861, 3862, 3863, 3864, 3865, 3866, 3867, 3868, 3869, 3870, 3871, 3872, 3873, 3874, 3875, 3876, 3877, 3878, 3879, 3880, 3881, 3882, 3883, 3884, 3885, 3886, 3887, 3888, 3889, 3890, 3891, 3892, 3893, 3894, 3895, 3896, 3897, 3898, 3899, 3900, 3901, 3902, 3903, 3904, 3905, 3906, 3907, 3908, 3909, 3910, 3911, 3912, 3913, 3914, 3915, 3916, 3917, 3918, 3919, 3920, 3921, 3922, 3923, 3924, 3925, 3926, 3927, 3928, 3929, 3930, 3931, 3932, 3933, 3934, 3935, 3936, 3937, 3938, 3939, 3940, 3941, 3942, 3943, 3944, 3945, 3946, 3947, 3948, 3949, 3950, 3951, 3952, 3953, 3954, 3955, 3956, 3957, 3958, 3959, 3960, 3961, 3962, 3963, 3964, 3965, 3966, 3967, 3968, 3969, 3970, 3971, 3972, 3973, 3974, 3975, 3976, 3977, 3978, 3979, 3980, 3981, 3982, 3983, 3984, 3985, 3986, 3987, 3988, 3989, 3990, 3991, 3992, 3993, 3994, 3995, 3996, 3997, 3998, 3999, 4000, 4001, 4002, 4003, 4004, 4005, 4006, 4007, 4008, 4009, 4010, 4011, 4012, 4013, 4014, 4015, 4016, 4017, 4018, 4019, 4020, 4021, 4022, 4023, 4024, 4025, 4026, 4027, 4028, 4029, 4030, 4031, 4032, 4033, 4034, 4035, 4036, 4037, 4038, 4039, 4040, 4041, 4042, 4043, 4044, 4045, 4046, 4047, 4048, 4049, 4050, 4051, 4052, 4053, 4054, 4055, 4056, 4057, 4058, 4059, 4060, 4061, 4062, 4063, 4064, 4065, 4066, 4067, 4068, 4069, 4070, 4071, 4072, 4073, 4074, 4075, 4076, 4077, 4078, 4079, 4080, 4081, 4082, 4083, 4084, 4085, 4086, 4087, 4088, 4089, 4090, 4091, 4092, 4093, 4094, 4095, 4096, 4097, 4098, 4099, 4100, 4101, 4102, 4103, 4104, 4105, 4106, 4107, 4108, 4109, 4110, 4111, 4112, 4113, 4114, 4115, 4116, 4117, 4118, 4119, 4120, 4121, 4122, 4123, 4124, 4125, 4126, 4127, 4128, 4129, 4130, 4131, 4132, 4133, 4134, 4135, 4136, 4137, 4138, 4139, 4140, 4141, 4142, 4143, 4144, 4145, 4146, 4147, 4148, 4149, 4150, 4151, 4152, 4153, 4154, 4155, 4156, 4157, 4158, 4159, 4160, 4161, 4162, 4163, 4164, 4165, 4166, 4167, 4168, 4169, 4170, 4171, 4172, 4173, 4174, 4175, 4176, 4177, 4178, 4179, 4180, 4181, 4182, 4183, 4184, 4185, 4186, 4187, 4188, 4189, 4190, 4191, 4192, 4193, 4194, 4195, 4196, 4197, 4198, 4199, 4200, 4201, 4202, 4203, 4204, 4205, 4206, 4207, 4208, 4209, 4210, 4211, 4212, 4213, 4214, 4215, 4216, 4217, 4218, 4219, 4220, 4221, 4222, 4223, 4224, 4225, 4226, 4227, 4228, 4229, 4230, 4231, 4232, 4233, 4234, 4235, 4236, 4237, 4238, 4239, 4240, 4241, 4242, 4243, 4244, 4245, 4246, 4247, 4248, 4249, 4250, 4251, 4252, 4253, 4254, 4255, 4256, 4257, 4258, 4259, 4260, 4261, 4262, 4263, 4264, 4265, 4266, 4267, 4268, 4269, 4270, 4271, 4272, 4273, 4274, 4275, 4276, 4277, 4278, 4279, 4280, 4281, 4282, 4283, 4284, 4285, 4286, 4287, 4288, 4289, 4290, 4291, 4292, 4293, 4294, 4295, 4296, 4297, 4298, 4299, 4300, 4301, 4302, 4303, 4304, 4305, 4306, 4307, 4308, 4309, 4310, 4311, 4312, 4313, 4314, 4315, 4316, 4317, 4318, 4319, 4320, 4321, 4322, 4323, 4324, 4325, 4326, 4327, 4328, 4329, 4330, 4331, 4332, 4333, 4334, 4335, 4336, 4337, 4338, 4339, 4340, 4341, 4342, 4343, 4344, 4345, 4346, 4347, 4348, 4349, 4350, 4351, 4352, 4353, 4354, 4355, 4356, 4357, 4358, 4359, 4360, 4361, 4362, 4363, 4364, 4365, 4366, 4367, 4368, 4369, 4370, 4371, 4372, 4373, 4374, 4375, 4376, 4377, 4378, 4379, 4380, 4381, 4382, 4383, 4384, 4385, 4386, 4387, 4388, 4389, 4390, 4391, 4392, 4393, 4394, 4395, 4396, 4397, 4398, 4399, 4400, 4401, 4402, 4403, 4404, 4405, 4406, 4407, 4408, 4409, 4410, 4411, 4412, 4413, 4414, 4415, 4416, 4417, 4418, 4419, 4420, 4421, 4422, 4423, 4424, 4425, 4426, 4427, 4428, 4429, 4430, 4431, 4432, 4433, 4434, 4435, 4436, 4437, 4438, 4439, 4440, 4441, 4442, 4443, 4444, 4445, 4446, 4447, 4448, 4449, 4450, 4451, 4452, 4453, 4454, 4455, 4456, 4457, 4458, 4459, 4460, 4461, 4462, 4463, 4464, 4465, 4466, 4467, 4468, 4469, 4470, 4471, 4472, 4473, 4474, 4475, 4476, 4477, 4478, 4479, 4480, 4481, 4482, 4483, 4484, 4485, 4486, 4487, 4488, 4489, 4490, 4491, 4492, 4493, 4494, 4495, 4496, 4497, 4498, 4499, 4500, 4501, or 4502.

The spacer sequence can comprise nucleotide 1 through nucleotide 16 of any one of SEQ ID NOs: 229-446 or 2497-4502. The spacer sequence can comprise nucleotide 1 through nucleotide 17 of any one of SEQ ID NOs: 229-446 or 2497-4502. The spacer sequence can comprise nucleotide 1 through nucleotide 18 of any one of SEQ ID NOs: 229-446 or 2497-4502. The spacer sequence can comprise nucleotide 1 through nucleotide 19 of any one of SEQ ID NOs: 229-446 or 2497-4502. The spacer sequence can comprise nucleotide 1 through nucleotide 20 of any one of SEQ ID NOs: 229-446 or 2497-4502. The spacer sequence can comprise nucleotide 1 through nucleotide 21 of any one of SEQ ID NOs: 229-446 or 2497-4502. The spacer sequence can comprise nucleotide 1 through nucleotide 22 of any one of SEQ ID NOS: 229-446 or 2497-4502. The spacer sequence can comprise nucleotide 1 through nucleotide 23 of any one of SEQ ID NOs: 229-446 or 2497-4502. The spacer sequence can comprise nucleotide 1 through nucleotide 24 of any one of SEQ ID NOs: 229-446 or 2497-4502. The spacer sequence can comprise nucleotide 1 through nucleotide 25 of any one of SEQ ID NOs: 229-446 or 2497-4502. The spacer sequence can comprise nucleotide 1 through nucleotide 26 of any one of SEQ ID NOs: 229-446 or 2497-4502. The spacer sequence can comprise nucleotide 1 through nucleotide 27 of any one of SEQ ID NOs: 229-446 or 2497-4502. The spacer sequence can comprise nucleotide 1 through nucleotide 28 of any one of SEQ ID NOs: 229-446 or 2497-4502. The spacer sequence can comprise nucleotide 1 through nucleotide 29 of any one of SEQ ID NOs: 229-446 or 2497-4502. The spacer sequence can comprise nucleotide 1 through nucleotide 30 of any one of SEQ ID NOs: 229-446 or 2497-4502.

In some embodiments, the spacer sequence has at least 90% identity (e.g., at least 90%, 91%, 92%, 93%, 94%, 95%, 96%, 97%, 98% or 99% identity) to a sequence of Table 5A or 5B or a portion of a sequence of Table 5A or 5B. The spacer sequence can have at least 90% identity to a sequence comprising nucleotide 1 through nucleotide 16 of any one of SEQ ID NOs: 229-446 or 2497-4502. The spacer sequence can have at least 90% identity to a sequence comprising nucleotide 1 through nucleotide 17 of any one of SEQ ID NOs: 229-446 or 2497-4502. The spacer sequence can have at least 90% identity to a sequence comprising nucleotide 1 through nucleotide 18 of any one of SEQ ID NOs: 229-446 or 2497-4502. The spacer sequence can have at least 90% identity to a sequence comprising nucleotide 1 through nucleotide 19 of any one of SEQ ID NOs: 229-446 or 2497-4502. The spacer sequence can have at least 90% identity to a sequence comprising nucleotide 1 through nucleotide 20 of any one of SEQ ID NOs: 229-446 or 2497-4502. The spacer sequence can have at least 90% identity to a sequence comprising nucleotide 1 through nucleotide 21 of any one of SEQ ID NOs: 229-446 or 2497-4502. The spacer sequence can have at least 90% identity to a sequence comprising nucleotide 1 through nucleotide 22 of any one of SEQ ID NOs: 229-446 or 2497-4502. The spacer sequence can have at least 90% identity to a sequence comprising nucleotide 1 through nucleotide 23 of any one of SEQ ID NOs: 229-446 or 2497-4502. The spacer sequence can have at least 90% identity to a sequence comprising nucleotide 1 through nucleotide 24 of any one of SEQ ID NOs: 229-446 or 2497-4502. The spacer sequence can have at least 90% identity to a sequence comprising nucleotide 1 through nucleotide 25 of any one of SEQ ID NOs: 229-446 or 2497-4502. The spacer sequence can have at least 90% identity to a sequence comprising nucleotide 1 through nucleotide 26 of any one of SEQ ID NOs: 229-446 or 2497-4502. The spacer sequence can have at least 90% identity to a sequence comprising nucleotide 1 through nucleotide 27 of any one of SEQ ID NOs: 229-446 or 2497-4502. The spacer sequence can have at least 90% identity to a sequence comprising nucleotide 1 through nucleotide 28 of any one of SEQ ID NOs: 229-446 or 2497-4502. The spacer sequence can have at least 90% identity to a sequence comprising nucleotide 1 through nucleotide 29 of any one of SEQ ID NOs: 229-446 or 2497-4502. The spacer sequence can have at least 90% identity to a sequence comprising nucleotide 1 through nucleotide 30 of any one of SEQ ID NOs: 229-446 or 2497-4502.

TABLE 5A
Target and Spacer Sequences-Exons

| ref_ | | | | | | |
|---|---|---|---|---|---|---|
| id | Strand | PAM | | target | | spacer |
| STMN2_ | + | TT | 1 | CCTTCGCCACTGCTCAGC | 2 | CCUUCGCCACUGCUCAGC |
| exon1 | | TG | 1 | GTCTGCACATCC | 2 | GUCUGCACAUCC |
| | | | | | 9 | |
| STMN2_ | + | CT | 1 | GCCACTGCTCAGCGTCTG | 2 | GCCACUGCUCAGCGUCU |
| exon1 | | TC | 2 | CACATCCCTACA | 3 | GCACAUCCCUACA |
| | | | | | 0 | |
| STMN2_ | + | CT | 1 | GCCTTCGCCACTGCTCAG | 2 | GCCUUCGCCACUGCUCAG |
| exon1 | | TT | 3 | CGTCTGCACATC | 3 | CGUCUGCACAUC |
| | | | | | 1 | |
| STMN2_ | − | CT | 1 | CCCATTGCTGTTTTAGCC | 2 | CCCAUUGCUGUUUUAGCC |
| exon1 | | TA | 4 | ATTGTAGGGATG | 3 | AUUGUAGGGAUG |
| | | | | | 2 | |
| STMN2_ | − | AT | 1 | CTGTTTTAGCCATTGTAG | 2 | CUGUUUUAGCCAUUGUAG |
| exon1 | | TG | 5 | GGATGTGCAGAC | 3 | GGAUGUGCAGAC |
| | | | | | 3 | |
| STMN2_ | − | GT | 1 | TAGCCATTGTAGGGATGT | 2 | UAGCCAUUGUAGGGAUGU |
| exon1 | | TT | 6 | GCAGACGCTGAG | 3 | GCAGACGCUGAG |
| | | | | | 4 | |
| STMN2_ | − | TT | 1 | AGCCATTGTAGGGATGTG | 2 | AGCCAUUGUAGGGAUGU |
| exon1 | | TT | 7 | CAGACGCTGAGC | 3 | GCAGACGCUGAGC |
| | | | | | 5 | |
| STMN2_ | − | TT | 1 | GCCATTGTAGGGATGTGC | 2 | GCCAUUGUAGGGAUGUG |
| exon1 | | TA | 8 | AGACGCTGAGCA | 3 | CAGACGCUGAGCA |
| | | | | | 6 | |
| STMN2_ | − | AT | 1 | TAGGGATGTGCAGACGCT | 2 | UAGGGAUGUGCAGACGC |
| exon1 | | TG | 9 | GAGCAGTGGCGA | 3 | UGAGCAGUGGCGA |
| | | | | | 7 | |
| STMN2_ | + | GT | 2 | TCCGTCGGCTCTACCTGG | 2 | UCCGUCGGCUCUACCUG |
| exon1 | | TC | 0 | AGCCCACCTCT | 3 | GAGCCCACCUCU |
| | | | | | 8 | |
| STMN2_ | − | TT | 2 | GTTTTCTAAGCCAGGGAG | 2 | GUUUUCUAAGCCAGGGAG |
| exon2 | | TG | 1 | GTTTTGAAAGAT | 3 | GUUUUGAAAGAU |
| | | | | | 9 | |
| STMN2_ | + | CT | 2 | CAAAACCTCCCTGGCTTA | 2 | CAAAACCUCCCUGGCUUA |
| exon2 | | TT | 2 | GAAAACCAAATT | 4 | GAAAACCAAAUU |
| | | | | | 0 | |
| STMN2_ | + | TT | 2 | AAAACCTCCCTGGCTTAG | 2 | AAAACCUCCCUGGCUUAG |
| exon2 | | TC | 3 | AAAACCAAATTT | 4 | AAAACCAAAUUU |
| | | | | | 1 | |
| STMN2_ | + | CT | 2 | GAAAACCAAATTTTTGTAG | 2 | GAAAACCAAAUUUUUGUA |
| exon2 | | TA | 4 | AGAGAGATGGG | 4 | GAGAGAGAUGGG |
| | | | | | 2 | |
| STMN2_ | − | AT | 2 | GGTTTTCTAAGCCAGGGA | 2 | GGUUUUCUAAGCCAGGGA |
| exon2 | | TT | 5 | GGTTTTGAAAGA | 4 | GGUUUUGAAAGA |
| | | | | | 3 | |
| STMN2_ | + | TT | 2 | TGTAGAGAGAGATGGGTA | 2 | UGUAGAGAGAGAUGGGUA |
| exon2 | | TT | 6 | GAATCTAATTTT | 4 | GAAUCUAAUUUU |
| | | | | | 4 | |
| STMN2_ | + | AT | 2 | TTGTAGAGAGAGATGGGT | 2 | UUGUAGAGAGAGAUGGG |
| exon2 | | TT | 7 | AGAATCTAATTT | 4 | UAGAAUCUAAUUU |
| | | | | | 5 | |
| STMN2_ | − | AT | 2 | GATTCTACCCATCTCTCTC | 2 | GAUUCUACCCAUCUCUCU |
| exon2 | | TA | 8 | TACAAAAATTT | 4 | CUACAAAAAUUU |
| | | | | | 6 | |
| STMN2_ | − | AT | 2 | TACCCATCTCTCTCTACAA | 2 | UACCCAUCUCUCUCUACA |
| exon2 | | TC | 9 | AAATTTGGTTT | 4 | AAAAUUUGGUUU |
| | | | | | 7 | |
| STMN2_ | − | TT | 3 | GAATAAAATTAGATTCTAC | 2 | GAAUAAAAUUAGAUUCUA |
| exon2 | | TA | 0 | CCATCTCTCTC | 4 | CCCAUCUCUCUC |
| | | | | | 8 | |
| STMN2_ | − | CT | 3 | AGAATAAAATTAGATTCTA | 2 | AGAAUAAAAUUAGAUUCU |
| exon2 | | TT | 1 | CCCATCTCTCT | 4 | ACCCAUCUCUCU |
| | | | | | 9 | |
| STMN2_ | − | AT | 3 | CTTTAGAATAAAATTAGAT | 2 | CUUUAGAAUAAAAUUAGA |
| exon2 | | TG | 2 | TCTACCCATCT | 5 | UUCUACCCAUCU |
| | | | | | 0 | |
| STMN2_ | + | AT | 3 | TAAAGCAATTAGCATTACA | 2 | UAAAGCAAUUAGCAUUAC |
| exon2 | | TC | 3 | TCATCACAGCA | 5 | AUCAUCACAGCA |
| | | | | | 1 | |
| STMN2_ | + | TT | 3 | TTCTAAAGCAATTAGCATT | 2 | UUCUAAAGCAAUUAGCAU |
| exon2 | | TA | 4 | ACATCATCACA | 5 | UACAUCAUCACA |
| | | | | | 2 | |
| STMN2_ | + | AT | 3 | TATTCTAAAGCAATTAGCA | 2 | UAUUCUAAAGCAAUUAGC |
| exon2 | | TT | 5 | TTACATCATCA | 5 | AUUACAUCAUCA |
| | | | | | 3 | |
| STMN2_ | + | TT | 3 | ATTCTAAAGCAATTAGCAT | 2 | AUUCUAAAGCAAUUAGCA |
| exon2 | | TT | 6 | TACATCATCAC | 5 | UUACAUCAUCAC |
| | | | | | 4 | |
| STMN2_ | + | TT | 3 | GTAGAGAGAGATGGGTAG | 2 | GUAGAGAGAGAUGGGUA |
| exon2 | | TT | 7 | AATCTAATTTTA | 5 | GAAUCUAAUUUUA |
| | | | | | 5 | |
| STMN2_ | + | TT | 3 | TAGAGAGAGATGGGTAGA | 2 | UAGAGAGAGAUGGGUAGA |
| exon2 | | TG | 8 | ATCTAATTTTAT | 5 | AUCUAAUUUUAU |
| | | | | | 6 | |
| STMN2_ | + | AT | 3 | GCATTACATCATCACAGC | 2 | GCAUUACAUCAUCACAGC |
| exon2 | | TA | 9 | AG | 5 | AG |
| | | | | | 7 | |
| STMN2_ | − | GT | 4 | TCTAAGCCAGGGAGGTTT | 2 | UCUAAGCCAGGGAGGUUU |
| exon2 | | TT | 0 | TGAAAGATT | 5 | UGAAAGAUU |
| | | | | | 8 | |
| STMN2_ | − | TT | 4 | CTAAGCCAGGGAGGTTTT | 2 | CUAAGCCAGGGAGGUUUU |
| exon2 | | TT | 1 | GAAAGATT | 5 | GAAAGAUU |
| | | | | | 9 | |
| STMN2_ | − | TT | 4 | TAAGCCAGGGAGGTTTTG | 2 | UAAGCCAGGGAGGUUUU |
| exon2 | | TC | 2 | AAAGATT | 6 | GAAAGAUU |
| | | | | | 0 | |
| STMN2_ | − | GT | 4 | CGAGGTTCCGGGTAAAAG | 2 | CGAGGUUCCGGGUAAAAG |
| exon3 | | TG | 3 | CAAGAGCAGATC | 6 | CAAGAGCAGAUC |
| | | | | | 1 | |
| STMN2_ | − | CT | 4 | TAGGCTGAAATGAAAAGC | 2 | UAGGCUGAAAUGAAAAGC |
| exon3 | | TG | 4 | TGAAGATTAGTA | 6 | UGAAGAUUAGUA |
| | | | | | 2 | |
| STMN2_ | − | GT | 4 | CGGGTAAAAGCAAGAGCA | 2 | CGGGUAAAAGCAAGAGCA |
| exon3 | | TC | 5 | GATCAGTGACAG | 6 | GAUCAGUGACAG |
| | | | | | 3 | |
| STMN2_ | − | TT | 4 | CCTTGTAGGCTGAAATGA | 2 | CCUUGUAGGCUGAAAUGA |
| exon3 | | TT | 6 | AAAGCTGAAGAT | 6 | AAAGCUGAAGAU |
| | | | | | 4 | |
| STMN2_ | − | TT | 4 | TCCTTGTAGGCTGAAATG | 2 | UCCUUGUAGGCUGAAAUG |
| exon3 | | TT | 7 | AAAAGCTGAAGA | 6 | AAAAGCUGAAGA |
| | | | | | 5 | |
| STMN2_ | − | TT | 4 | TTCCTTGTAGGCTGAAAT | 2 | UUCCUUGUAGGCUGAAAU |
| exon3 | | TT | 8 | GAAAAGCTGAAG | 6 | GAAAAGCUGAAG |
| | | | | | 6 | |
| STMN2_ | − | AT | 4 | TTTCCTTGTAGGCTGAAAT | 2 | UUUCCUUGUAGGCUGAAA |
| exon3 | | TT | 9 | GAAAAGCTGAA | 6 | UGAAAAGCUGAA |
| | | | | | 7 | |
| STMN2_ | − | CT | 5 | ATTTTTTCCTTGTAGGCTG | 2 | AUUUUUUCCUUGUAGGCU |
| exon3 | | TC | 0 | AAATGAAAAGC | 6 | GAAAUGAAAAGC |
| | | | | | 8 | |
| STMN2_ | + | AT | 5 | AGAAAAAATGAAATATACT | 2 | AGAAAAAAUGAAAUAUAC |
| exon3 | | TC | 1 | AATCTTCAGCT | 6 | UAAUCUUCAGCU |
| | | | | | 9 | |
| STMN2_ | + | CT | 5 | AGCTTTTCATTTCAGCCTA | 2 | AGCUUUUCAUUUCAGCCU |
| exon3 | | TC | 2 | CAAGGAAAAAA | 7 | ACAAGGAAAAAA |
| | | | | | 0 | |
| STMN2_ | + | CT | 5 | TCATTTCAGCCTACAAGG | 2 | UCAUUUCAGCCUACAAGG |
| exon3 | | TT | 3 | AAAAAATGAAGG | 7 | AAAAAAUGAAGG |
| | | | | | 1 | |
| STMN2_ | − | TT | 5 | CTTGTAGGCTGAAATGAA | 2 | CUUGUAGGCUGAAAUGAA |
| exon3 | | TC | 4 | AAGCTGAAGATT | 7 | AAGCUGAAGAUU |
| | | | | | 2 | |
| STMN2_ | + | TT | 5 | ATTTCAGCCTACAAGGAA | 2 | AUUUCAGCCUACAAGGAA |
| exon3 | | TC | 5 | AAAATGAAGGAG | 7 | AAAAUGAAGGAG |
| | | | | | 3 | |
| STMN2_ | + | TT | 5 | CATTTCAGCCTACAAGGA | 2 | CAUUUCAGCCUACAAGGA |
| exon3 | | TT | 6 | AAAAATGAAGGA | 7 | AAAAAUGAAGGA |
| | | | | | 4 | |
| STMN2_ | − | GT | 5 | CTCACCATCGTAAGTATA | 2 | CUCACCAUCGUAAGUAUA |
| exon3 | | TA | 7 | GATGTTGATGTT | 7 | GAUGUUGAUGUU |
| | | | | | 5 | |
| STMN2_ | − | TT | 5 | CAAATGATCTAGCTAGCA | 2 | CAAAUGAUCUAGCUAGCA |
| exon3 | | TC | 8 | GGGGTATGTCTA | 7 | GGGGUAUGUCUA |
| | | | | | 6 | |
| STMN2_ | − | CT | 5 | CCAAATGATCTAGCTAGC | 2 | CCAAAUGAUCUAGCUAGC |
| exon3 | | TT | 9 | AGGGGTATGTCT | 7 | AGGGGUAUGUCU |
| | | | | | 7 | |
| STMN2_ | + | CT | 6 | CGATGGTGAGTAACCTAG | 2 | CGAUGGUGAGUAACCUAG |
| exon3 | | TA | 0 | GATAGACATACC | 7 | GAUAGACAUACC |
| | | | | | 8 | |
| STMN2_ | + | TT | 6 | CCCGGAACCTCGCAACAT | 2 | CCCGGAACCUCGCAACAU |
| exon3 | | TA | 1 | CAACATCTATAC | 7 | CAACAUCUAUAC |
| | | | | | 9 | |
| STMN2_ | − | GT | 6 | ATGTTGCGAGGTTCCGGG | 2 | AUGUUGCGAGGUUCCGG |
| exon3 | | TG | 2 | TAAAAGCAAGAG | 8 | GUAAAAGCAAGAG |
| | | | | | 0 | |
| STMN2_ | + | CT | 6 | TACCCGGAACCTCGCAAC | 2 | UACCCGGAACCUCGCAAC |
| exon3 | | TT | 3 | ATCAACATCTAT | 8 | AUCAACAUCUAU |
| | | | | | 1 | |
| STMN2_ | + | CT | 6 | CTTTTACCCGGAACCTCG | 2 | CUUUUACCCGGAACCUCG |
| exon3 | | TG | 4 | CAACATCAACAT | 8 | CAACAUCAACAU |
| | | | | | 2 | |
| STMN2_ | + | TT | 6 | AGCCTACAAGGAAAAAAT | 2 | AGCCUACAAGGAAAAAAU |
| exon3 | | TC | 5 | GAAGGAGCTGTC | 8 | GAAGGAGCUGUC |
| | | | | | 3 | |
| STMN2_ | + | AT | 6 | CAGCCTACAAGGAAAAAA | 2 | CAGCCUACAAGGAAAAAA |
| exon3 | | TT | 6 | TGAAGGAGCTGT | 8 | UGAAGGAGCUGU |
| | | | | | 4 | |
| STMN2_ | + | TT | 6 | ACCCGGAACCTCGCAACA | 2 | ACCCGGAACCUCGCAACA |
| exon3 | | TT | 7 | TCAACATCTATA | 8 | UCAACAUCUAUA |
| | | | | | 5 | |
| STMN2_ | − | AT | 6 | GTATATTTCATTTTTTCTG | 2 | GUAUAUUUCAUUUUUUCU |
| exon3 | | TA | 8 | AATTTCTC | 8 | GAAUUUCUC |
| | | | | | 6 | |
| STMN2_ | − | TT | 6 | TTCTGGATCTCCTCCAGG | 2 | UUCUGGAUCUCCUCCAGG |
| exon4 | | TC | 9 | GACAGGTCTTTC | 8 | GACAGGUCUUUC |
| | | | | | 7 | |
| STMN2_ | − | CT | 7 | TGGATCTCCTCCAGGGAC | 2 | UGGAUCUCCUCCAGGGAC |
| exon4 | | TC | 0 | AGGTCTTTCTTC | 8 | AGGUCUUUCUUC |
| | | | | | 8 | |
| STMN2_ | − | CT | 7 | CTTCTTTGGAGAAGCTAA | 2 | CUUCUUUGGAGAAGCUAA |
| exon4 | | TT | 1 | AGTTCGTGGGGC | 8 | AGUUCGUGGGGC |
| | | | | | 9 | |
| STMN2_ | − | TT | 7 | TTCTTTGGAGAAGCTAAA | 2 | UUCUUUGGAGAAGCUAAA |
| exon4 | | TC | 2 | GTTCGTGGGGCT | 9 | GUUCGUGGGGCU |
| | | | | | 0 | |
| STMN2_ | − | CT | 7 | TTTGGAGAAGCTAAAGTT | 2 | UUUGGAGAAGCUAAAGUU |
| exon4 | | TC | 3 | CGTGGGGCTTCT | 9 | CGUGGGGCUUCU |
| | | | | | 1 | |
| STMN2_ | − | CT | 7 | GGAGAAGCTAAAGTTCGT | 2 | GGAGAAGCUAAAGUUCGU |
| exon4 | | TT | 4 | GGGGCTTCTGAG | 9 | GGGGCUUCUGAG |
| | | | | | 2 | |
| STMN2_ | − | TT | 7 | GAGAAGCTAAAGTTCGTG | 2 | GAGAAGCUAAAGUUCGUG |
| exon4 | | TG | 5 | GGGCTTCTGAGA | 9 | GGGCUUCUGAGA |
| | | | | | 3 | |
| STMN2_ | − | GT | 7 | GTGGGGCTTCTGAGATAG | 2 | GUGGGGCUUCUGAGAUA |
| exon4 | | TC | 6 | GAGATGGTGGCT | 9 | GGAGAUGGUGGCU |
| | | | | | 4 | |
| STMN2_ | − | CT | 7 | TGAGATAGGAGATGGTGG | 2 | UGAGAUAGGAGAUGGUG |
| exon4 | | TC | 7 | CTTCAAGATCAG | 9 | GCUUCAAGAUCAG |
| | | | | | 5 | |
| STMN2_ | − | TT | 7 | TTGATTTGCTTCACTTCCA | 2 | UUGAUUUGCUUCACUUCC |
| exon4 | | TG | 8 | TATCTGAAAAG | 9 | AUAUCUGAAAAG |
| | | | | | 6 | |
| STMN2_ | − | GT | 7 | GTTGATTTGCTTCACTTCC | 2 | GUUGAUUUGCUUCACUUC |
| exon4 | | TT | 9 | ATATCTGAAAA | 9 | CAUAUCUGAAAA |
| | | | | | 7 | |
| STMN2_ | − | GT | 8 | ATTTGCTTCACTTCCATAT | 2 | AUUUGCUUCACUUCCAUA |
| exon4 | | TG | 0 | CTGAAAAGTGA | 9 | UCUGAAAAGUGA |
| | | | | | 8 | |
| STMN2_ | − | AT | 8 | GCTTCACTTCCATATCTGA | 2 | GCUUCACUUCCAUAUCUG |
| exon4 | | TT | 1 | AAAGTGAACAT | 9 | AAAAGUGAACAU |
| | | | | | 9 | |
| STMN2_ | − | TT | 8 | CTTCACTTCCATATCTGAA | 3 | CUUCACUUCCAUAUCUGA |
| exon4 | | TG | 2 | AAGTGAACATT | 0 | AAAGUGAACAUU |
| | | | | | 0 | |
| STMN2_ | − | CT | 8 | ACTTCCATATCTGAAAAGT | 3 | ACUUCCAUAUCUGAAAAG |
| exon4 | | TC | 3 | GAACATTTGAG | 0 | UGAACAUUUGAG |
| | | | | | 1 | |
| STMN2_ | − | CT | 8 | CATATCTGAAAAGTGAAC | 3 | CAUAUCUGAAAAGUGAAC |
| exon4 | | TC | 4 | ATTTGAGAATGT | 0 | AUUUGAGAAUGU |
| | | | | | 2 | |
| STMN2_ | − | AT | 8 | GAGAATGTTAAGCATACA | 3 | GAGAAUGUUAAGCAUACA |
| exon4 | | TT | 5 | AAGCTTGCAGCA | 0 | AAGCUUGCAGCA |
| | | | | | 3 | |
| STMN2_ | − | TT | 8 | AGAATGTTAAGCATACAAA | 3 | AGAAUGUUAAGCAUACAA |
| exon4 | | TG | 6 | GCTTGCAGCAT | 0 | AGCUUGCAGCAU |
| | | | | | 4 | |
| STMN2_ | − | GT | 8 | CTTCTGGATCTCCTCCAG | 3 | CUUCUGGAUCUCCUCCAG |
| exon4 | | TT | 7 | GGACAGGTCTTT | 0 | GGACAGGUCUUU |
| | | | | | 5 | |
| STMN2_ | − | CT | 8 | AAGATCAGCTCAAAAGCC | 3 | AAGAUCAGCUCAAAAGCC |
| exon4 | | TC | 8 | TGGCCAGAGGCA | 0 | UGGCCAGAGGCA |
| | | | | | 6 | |
| STMN2_ | − | TT | 8 | CTCTGCAGCCTCCAGTTT | 3 | CUCUGCAGCCUCCAGUUU |
| exon4 | | TC | 9 | CTTCTGGATCTC | 0 | CUUCUGGAUCUC |
| | | | | | 7 | |
| STMN2_ | + | TT | 9 | AGATATGGAAGTGAAGCA | 3 | AGAUAUGGAAGUGAAGCA |
| exon4 | | TC | 0 | AATCAACAAACG | 0 | AAUCAACAAACG |
| | | | | | 8 | |
| STMN2_ | − | CT | 9 | TTTCCTCTGCAGCCTCCA | 3 | UUUCCUCUGCAGCCUCCA |
| exon4 | | TC | 1 | GTTTCTTCTGGA | 0 | GUUUCUUCUGGA |
| | | | | | 9 | |
| STMN2_ | + | TT | 9 | TATGCTTAACATTCTCAAA | 3 | UAUGCUUAACAUUCUCAA |
| exon4 | | TG | 2 | TGTTCACTTTT | 1 | AUGUUCACUUUU |
| | | | | | 0 | |
| STMN2_ | + | CT | 9 | ACATTCTCAAATGTTCACT | 3 | ACAUUCUCAAAUGUUCAC |
| exon4 | | TA | 3 | TTTCAGATATG | 1 | UUUUCAGAUAUG |
| | | | | | 1 | |
| STMN2_ | + | AT | 9 | TCAAATGTTCACTTTTCAG | 3 | UCAAAUGUUCACUUUUCA |
| exon4 | | TC | 4 | ATATGGAAGTG | 1 | GAUAUGGAAGUG |
| | | | | | 2 | |
| STMN2_ | + | GT | 9 | ACTTTTCAGATATGGAAGT | 3 | ACUUUUCAGAUAUGGAAG |
| exon4 | | TC | 5 | GAAGCAAATCA | 1 | UGAAGCAAAUCA |
| | | | | | 3 | |
| STMN2_ | + | CT | 9 | TCAGATATGGAAGTGAAG | 3 | UCAGAUAUGGAAGUGAAG |
| exon4 | | TT | 6 | CAAATCAACAAA | 1 | CAAAUCAACAAA |
| | | | | | 4 | |
| STMN2_ | + | TT | 9 | CAGATATGGAAGTGAAGC | 3 | CAGAUAUGGAAGUGAAGC |
| exon4 | | TT | 7 | AAATCAACAAAC | 1 | AAAUCAACAAAC |
| | | | | | 5 | |
| STMN2_ | + | CT | 9 | TGAGCTGATCTTGAAGCC | 3 | UGAGCUGAUCUUGAAGCC |
| exon4 | | TT | 8 | ACCATCTCCTAT | 1 | ACCAUCUCCUAU |
| | | | | | 6 | |
| STMN2_ | + | TT | 9 | GAGCTGATCTTGAAGCCA | 3 | GAGCUGAUCUUGAAGCCA |
| exon4 | | TT | 9 | CCATCTCCTATC | 1 | CCAUCUCCUAUC |
| | | | | | 7 | |
| STMN2_ | + | TT | 1 | AGCTGATCTTGAAGCCAC | 3 | AGCUGAUCUUGAAGCCAC |
| exon4 | | TG | 0 | CATCTCCTATCT | 1 | CAUCUCCUAUCU |
| | | | 0 | | 8 | |
| STMN2_ | + | CT | 1 | AAGCCACCATCTCCTATC | 3 | AAGCCACCAUCUCCUAUC |
| exon4 | | TG | 0 | TCAGAAGCCCCA | 1 | UCAGAAGCCCCA |
| | | | 1 | | 9 | |
| STMN2_ | + | CT | 1 | AGCTTCTCCAAAGAAGAA | 3 | AGCUUCUCCAAAGAAGAA |
| exon4 | | TT | 0 | AGACCTGTCCCT | 2 | AGACCUGUCCCU |
| | | | 2 | | 0 | |
| STMN2_ | + | TT | 1 | GCTTCTCCAAAGAAGAAA | 3 | GCUUCUCCAAAGAAGAAA |
| exon4 | | TA | 0 | GACCTGTCCCTG | 2 | GACCUGUCCCUG |
| | | | 3 | | 1 | |
| STMN2_ | + | CT | 1 | TCCAAAGAAGAAAGACCT | 3 | UCCAAAGAAGAAAGACCU |
| exon4 | | TC | 0 | GTCCCTGGAGGA | 2 | GUCCCUGGAGGA |
| | | | 4 | | 2 | |
| STMN2_ | + | CT | 1 | TTCCATAGGTTTTCCTTCT | 3 | UUCCAUAGGUUUUCCUUC |
| exon4 | | TT | 0 | CTCTCTCCCTC | 2 | UCUCUCUCCCUC |
| | | | 5 | | 3 | |
| STMN2_ | + | TT | 1 | TCCATAGGTTTTCCTTCTC | 3 | UCCAUAGGUUUUCCUUCU |
| exon4 | | TT | 0 | TCTCTCCCTCC | 2 | CUCUCUCCCUCC |
| | | | 6 | | 4 | |
| STMN2_ | + | TT | 1 | CCATAGGTTTTCCTTCTCT | 3 | CCAUAGGUUUUCCUUCUC |
| exon4 | | TT | 0 | CTCTCCCTCCC | 2 | UCUCUCCCUCCC |
| | | | 7 | | 5 | |
| STMN2_ | + | TT | 1 | CATAGGTTTTCCTTCTCTC | 3 | CAUAGGUUUUCCUUCUCU |
| exon4 | | TC | 0 | TCTCCCTCCCC | 2 | CUCUCCCUCCCC |
| | | | 8 | | 6 | |
| STMN2_ | + | GT | 1 | TCCTTCTCTCTCTCCCTCC | 3 | UCCUUCUCUCUCUCCCUC |
| exon4 | | TT | 0 | CCTGCTCCTCC | 2 | CCCUGCUCCUCC |
| | | | 9 | | 7 | |
| STMN2_ | − | GT | 1 | CCTTTCTTCTTTCCTCTGC | 3 | CCUUUCUUCUUUCCUCUG |
| exon4 | | TA | 1 | AGCCTCCAGTT | 2 | CAGCCUCCAGUU |
| | | | 0 | | 8 | |
| STMN2_ | − | CT | 1 | CTTCTTTCCTCTGCAGCCT | 3 | CUUCUUUCCUCUGCAGCC |
| exon4 | | TT | 1 | CCAGTTTCTTC | 2 | UCCAGUUUCUUC |
| | | | 1 | | 9 | |
| STMN2_ | − | TT | 1 | TTCTTTCCTCTGCAGCCTC | 3 | UUCUUUCCUCUGCAGCCU |
| exon4 | | TC | 1 | CAGTTTCTTCT | 3 | CCAGUUUCUUCU |
| | | | 2 | | 0 | |
| STMN2_ | − | CT | 1 | CCTCTGCAGCCTCCAGTT | 3 | CCUCUGCAGCCUCCAGUU |
| exon4 | | TT | 1 | TCTTCTGGATCT | 3 | UCUUCUGGAUCU |
| | | | 3 | | 1 | |
| STMN2_ | + | CT | 1 | GTATGCTTAACATTCTCAA | 3 | GUAUGCUUAACAUUCUCA |
| exon4 | | TT | 1 | ATGTTCACTTT | 3 | AAUGUUCACUUU |
| | | | 4 | | 2 | |
| STMN2_ | + | TT | 1 | CCTTCTCTCTCTCCCTCC | 3 | CCUUCUCUCUCUCCCUCC |
| exon4 | | TT | 1 | CCTGCTCCTCC | 3 | CCUGCUCCUCC |
| | | | 5 | | 3 | |
| STMN2_ | + | TT | 1 | CTTCTCTCTCTCCCTCCC | 3 | CUUCUCUCUCUCCCUCCC |
| exon4 | | TC | 1 | CTGCTCCTCC | 3 | CUGCUCCUCC |
| | | | 6 | | 4 | |
| STMN2_ | + | CT | 1 | TCTCTCTCCCTCCCCTGC | 3 | UCUCUCUCCCUCCCCUGC |
| exon4 | | TC | 1 | TCCTCC | 3 | UCCUCC |
| | | | 7 | | 5 | |
| STMN2_ | − | GT | 1 | AGCATACAAAGCTTGCAG | 3 | AGCAUACAAAGCUUGCAG |
| exon4 | | TA | 1 | CATGG | 3 | CAUGG |
| | | | 8 | | 6 | |
| STMN2_ | + | GT | 1 | GTGTTTGGATAATTATAAG | 3 | GUGUUUGGAUAAUUAUAA |
| exon5 | | TT | 1 | ATGGCTATGTT | 3 | GAUGGCUAUGUU |
| | | | 9 | | 7 | |
| STMN2_ | − | TT | 1 | CTGCAGACGTTCAATAAT | 3 | CUGCAGACGUUCAAUAAU |
| exon5 | | TC | 2 | AGCAGCTAGATT | 3 | AGCAGCUAGAUU |
| | | | 0 | | 8 | |
| STMN2_ | − | TT | 1 | AGGATCAGCTTTTCCTCC | 3 | AGGAUCAGCUUUUCCUCC |
| exon5 | | TC | 2 | GCCATCTTGCTG | 3 | GCCAUCUUGCUG |
| | | | 1 | | 9 | |
| STMN2_ | − | CT | 1 | TCCTCCGCCATCTTGCTG | 3 | UCCUCCGCCAUCUUGCUG |
| exon5 | | TT | 2 | AAGTTGTTGTTC | 4 | AAGUUGUUGUUC |
| | | | 2 | | 0 | |
| STMN2_ | − | TT | 1 | CCTCCGCCATCTTGCTGA | 3 | CCUCCGCCAUCUUGCUGA |
| exon5 | | TT | 2 | AGTTGTTGTTCT | 4 | AGUUGUUGUUCU |
| | | | 3 | | 1 | |
| STMN2_ | − | TT | 1 | CTCCGCCATCTTGCTGAA | 3 | CUCCGCCAUCUUGCUGAA |
| exon5 | | TC | 2 | GTTGTTGTTCTC | 4 | GUUGUUGUUCUC |
| | | | 4 | | 2 | |
| STMN2_ | − | CT | 1 | CTGAAGTTGTTGTTCTCCT | 3 | CUGAAGUUGUUGUUCUCC |
| exon5 | | TG | 2 | CCAAAGCCTTC | 4 | UCCAAAGCCUUC |
| | | | 5 | | 3 | |
| STMN2_ | − | GT | 1 | TTGTTCTCCTCCAAAGCCT | 3 | UUGUUCUCCUCCAAAGCC |
| exon5 | | TG | 2 | TCTGAAGGACT | 4 | UUCUGAAGGACU |
| | | | 6 | | 4 | |
| STMN2_ | − | GT | 1 | TTCTCCTCCAAAGCCTTCT | 3 | UUCUCCUCCAAAGCCUUC |
| exon5 | | TG | 2 | GAAGGACTTCT | 4 | UGAAGGACUUCU |
| | | | 7 | | 5 | |
| STMN2_ | − | GT | 1 | TCCTCCAAAGCCTTCTGA | 3 | UCCUCCAAAGCCUUCUGA |
| exon5 | | TC | 2 | AGGACTTCTCGC | 4 | AGGACUUCUCGC |
| | | | 8 | | 6 | |
| STMN2_ | − | CT | 1 | TGAAGGACTTCTCGCTCG | 3 | UGAAGGACUUCUCGCUCG |
| exon5 | | TC | 2 | TGTTCCCTCTTC | 4 | UGUUCCCUCUUC |
| | | | 9 | | 7 | |
| STMN2_ | − | TT | 1 | CAGGATCAGCTTTTCCTC | 3 | CAGGAUCAGCUUUUCCUC |
| exon5 | | TT | 3 | CGCCATCTTGCT | 4 | CGCCAUCUUGCU |
| | | | 0 | | 8 | |
| STMN2_ | − | CT | 1 | TCGCTCGTGTTCCCTCTT | 3 | UCGCUCGUGUUCCCUCU |
| exon5 | | TC | 3 | CTCTGCCAATTG | 4 | UCUCUGCCAAUUG |
| | | | 1 | | 9 | |
| STMN2_ | − | CT | 1 | TCTGCCAATTGTTTCAGCA | 3 | UCUGCCAAUUGUUUCAGC |
| exon5 | | TC | 3 | CCTGGGCCTCC | 5 | ACCUGGGCCUCC |
| | | | 2 | | 0 | |
| STMN2_ | − | AT | 1 | TTTCAGCACCTGGGCCTC | 3 | UUUCAGCACCUGGGCCUC |
| exon5 | | TG | 3 | CTGAGACTGGGG | 5 | CUGAGACUGGGG |
| | | | 3 | | 1 | |
| STMN2_ | − | GT | 1 | CAGCACCTGGGCCTCCTG | 3 | CAGCACCUGGGCCUCCU |
| exon5 | | TT | 3 | AGACTGGGGAAG | 5 | GAGACUGGGGAAG |
| | | | 4 | | 2 | |
| STMN2_ | − | TT | 1 | AGCACCTGGGCCTCCTGA | 3 | AGCACCUGGGCCUCCUGA |
| exon5 | | TC | 3 | GACTGGGGAAGA | 5 | GACUGGGGAAGA |
| | | | 5 | | 3 | |
| STMN2_ | − | GT | 1 | AATAATAGCAGCTAGATTA | 3 | AAUAAUAGCAGCUAGAUU |
| exon5 | | TC | 3 | GCCTCACGGTT | 5 | AGCCUCACGGUU |
| | | | 6 | | 4 | |
| STMN2_ | − | TT | 1 | CCTGCAGACGTTCAATAA | 3 | CCUGCAGACGUUCAAUAA |
| exon5 | | TT | 3 | TAGCAGCTAGAT | 5 | UAGCAGCUAGAU |
| | | | 7 | | 5 | |
| STMN2_ | − | CT | 1 | TCCTGCAGACGTTCAATA | 3 | UCCUGCAGACGUUCAAUA |
| exon5 | | TT | 3 | ATAGCAGCTAGA | 5 | AUAGCAGCUAGA |
| | | | 8 | | 6 | |
| STMN2_ | − | AT | 1 | CCTTTTCCTGCAGACGTT | 3 | CCUUUUCCUGCAGACGUU |
| exon5 | | TA | 3 | CAATAATAGCAG | 5 | CAAUAAUAGCAG |
| | | | 9 | | 7 | |
| STMN2_ | + | AT | 1 | AACGTCTGCAGGAAAAGG | 3 | AACGUCUGCAGGAAAAGG |
| exon5 | | TG | 4 | TAATCTCAGCAG | 5 | UAAUCUCAGCAG |
| | | | 0 | | 8 | |
| STMN2_ | − | GT | 1 | CCTCTTCTCTGCCAATTGT | 3 | CCUCUUCUCUGCCAAUUG |
| exon5 | | TC | 4 | TTCAGCACCTG | 5 | UUUCAGCACCUG |
| | | | 1 | | 9 | |
| STMN2_ | − | AT | 1 | TCAGGATCAGCTTTTCCT | 3 | UCAGGAUCAGCUUUUCCU |
| exon5 | | TT | 4 | CCGCCATCTTGC | 6 | CCGCCAUCUUGC |
| | | | 2 | | 0 | |
| STMN2_ | − | GT | 1 | CATTTTCAGGATCAGCTTT | 3 | CAUUUUCAGGAUCAGCUU |
| exon5 | | TC | 4 | TCCTCCGCCAT | 6 | UUCCUCCGCCAU |
| | | | 3 | | 1 | |
| STMN2_ | + | CT | 1 | AGAAGGCTTTGGAGGAGA | 3 | AGAAGGCUUUGGAGGAGA |
| exon5 | | TC | 4 | ACAACAACTTCA | 6 | ACAACAACUUCA |
| | | | 4 | | 2 | |
| STMN2_ | + | TT | 1 | GATAATTATAAGATGGCTA | 3 | GAUAAUUAUAAGAUGGCU |
| exon5 | | TG | 4 | TGTTTTTCTTC | 6 | AUGUUUUUCUUC |
| | | | 5 | | 3 | |
| STMN2_ | + | AT | 1 | TAAGATGGCTATGTTTTTC | 3 | UAAGAUGGCUAUGUUUUU |
| exon5 | | TA | 4 | TTCCCCAGTCT | 6 | CUUCCCCAGUCU |
| | | | 6 | | 4 | |
| STMN2_ | + | GT | 1 | TTCTTCCCCAGTCTCAGG | 3 | UUCUUCCCCAGUCUCAGG |
| exon5 | | TT | 4 | AGGCCCAGGTGC | 6 | AGGCCCAGGUGC |
| | | | 7 | | 5 | |
| STMN2_ | + | TT | 1 | TCTTCCCCAGTCTCAGGA | 3 | UCUUCCCCAGUCUCAGGA |
| exon5 | | TT | 4 | GGCCCAGGTGCT | 6 | GGCCCAGGUGCU |
| | | | 8 | | 6 | |
| STMN2_ | + | TT | 1 | CTTCCCCAGTCTCAGGAG | 3 | CUUCCCCAGUCUCAGGAG |
| exon5 | | TT | 4 | GCCCAGGTGCTG | 6 | GCCCAGGUGCUG |
| | | | 9 | | 7 | |
| STMN2_ | + | TT | 1 | TTCCCCAGTCTCAGGAGG | 3 | UUCCCCAGUCUCAGGAGG |
| exon5 | | TC | 5 | CCCAGGTGCTGA | 6 | CCCAGGUGCUGA |
| | | | 0 | | 8 | |
| STMN2_ | + | CT | 1 | CCCAGTCTCAGGAGGCCC | 3 | CCCAGUCUCAGGAGGCCC |
| exon5 | | TC | 5 | AGGTGCTGAAAC | 6 | AGGUGCUGAAAC |
| | | | 1 | | 9 | |
| STMN2_ | + | AT | 1 | GCAGAGAAGAGGGAACA | 3 | GCAGAGAAGAGGGAACAC |
| exon5 | | TG | 5 | CGAGCGAGAAGTC | 7 | GAGCGAGAAGUC |
| | | | 2 | | 0 | |
| STMN2_ | − | TT | 1 | TTCCATTTTCAGGATCAGC | 3 | UUCCAUUUUCAGGAUCAG |
| exon5 | | TG | 5 | TTTTCCTCCGC | 7 | CUUUUCCUCCGC |
| | | | 3 | | 1 | |
| STMN2_ | + | GT | 1 | GGATAATTATAAGATGGC | 3 | GGAUAAUUAUAAGAUGGC |
| exon5 | | TT | 5 | TATGTTTTTCTT | 7 | UAUGUUUUUCUU |
| | | | 4 | | 2 | |
| STMN2_ | + | CT | 1 | GGAGGAGAACAACAACTT | 3 | GGAGGAGAACAACAACUU |
| exon5 | | TT | 5 | CAGCAAGATGGC | 7 | CAGCAAGAUGGC |
| | | | 5 | | 3 | |
| STMN2_ | + | CT | 1 | AGCAAGATGGCGGAGGA | 3 | AGCAAGAUGGCGGAGGAA |
| exon5 | | TC | 5 | AAAGCTGATCCTG | 7 | AAGCUGAUCCUG |
| | | | 6 | | 4 | |
| STMN2_ | + | AT | 1 | AGGAAAACCGTGAGGCTA | 3 | AGGAAAACCGUGAGGCUA |
| exon5 | | TA | 5 | ATCTAGCTGCTA | 7 | AUCUAGCUGCUA |
| | | | 7 | | 5 | |
| STMN2_ | + | AT | 1 | TTGAACGTCTGCAGGAAA | 3 | UUGAACGUCUGCAGGAAA |
| exon5 | | TA | 5 | AGGTAATCTCAG | 7 | AGGUAAUCUCAG |
| | | | 8 | | 6 | |
| STMN2_ | − | AT | 1 | GCCTCACGGTTTTCCTTA | 3 | GCCUCACGGUUUUCCUUA |
| exon5 | | TA | 5 | ATTTGTTCCATT | 7 | AUUUGUUCCAUU |
| | | | 9 | | 7 | |
| STMN2_ | − | GT | 1 | TCCTTAATTTGTTCCATTT | 3 | UCCUUAAUUUGUUCCAUU |
| exon5 | | TT | 6 | TCAGGATCAGC | 7 | UUCAGGAUCAGC |
| | | | 0 | | 8 | |
| STMN2_ | − | TT | 1 | CCTTAATTTGTTCCATTTT | 3 | CCUUAAUUUGUUCCAUUU |
| exon5 | | TT | 6 | CAGGATCAGCT | 7 | UCAGGAUCAGCU |
| | | | 1 | | 9 | |
| STMN2_ | − | TT | 1 | CTTAATTTGTTCCATTTTC | 3 | CUUAAUUUGUUCCAUUUU |
| exon5 | | TC | 6 | AGGATCAGCTT | 8 | CAGGAUCAGCUU |
| | | | 2 | | 0 | |
| STMN2_ | − | CT | 1 | ATTTGTTCCATTTTCAGGA | 3 | AUUUGUUCCAUUUUCAGG |
| exon5 | | TA | 6 | TCAGCTTTTCC | 8 | AUCAGCUUUUCC |
| | | | 3 | | 1 | |
| STMN2_ | − | AT | 1 | GTTCCATTTTCAGGATCA | 3 | GUUCCAUUUUCAGGAUCA |
| exon5 | | TT | 6 | GCTTTTCCTCCG | 8 | GCUUUUCCUCCG |
| | | | 4 | | 2 | |
| STMN2_ | + | TT | 1 | GAGGAGAACAACAACTTC | 3 | GAGGAGAACAACAACUUC |
| exon5 | | TG | 6 | AGCAAGATGGCG | 8 | AGCAAGAUGGCG |
| | | | 5 | | 3 | |
| STMN2_ | + | TT | 1 | TGTTTGGATAATTATAAGA | 3 | UGUUUGGAUAAUUAUAAG |
| exon5 | | TG | 6 | TGGCTATGTTT | 8 | AUGGCUAUGUUU |
| | | | 6 | | 4 | |
| STMN2_ | − | CT | 1 | TAATTATCCAAACACAAAC | 3 | UAAUUAUCCAAACACAAA |
| exon5 | | TA | 6 | CTAG | 8 | CCUAG |
| | | | 7 | | 5 | |
| STMN2_ | − | GT | 1 | AGAAGAAATAAACTTGAC | 3 | AGAAGAAAUAAACUUGAC |
| exon6 | | TC | 6 | CAGCTATAAAGT | 8 | CAGCUAUAAAGU |
| | | | 8 | | 6 | |
| STMN2_ | − | CT | 1 | TCGTTAAACTCTATTAATC | 3 | UCGUUAAACUCUAUUAAU |
| exon6 | | TA | 6 | TCAAGGAGTCT | 8 | CUCAAGGAGUCU |
| | | | 9 | | 7 | |
| STMN2_ | − | TT | 1 | GTTCAGAAGAAATAAACTT | 3 | GUUCAGAAGAAAUAAACU |
| exon6 | | TA | 7 | GACCAGCTATA | 8 | UGACCAGCUAUA |
| | | | 0 | | 8 | |
| STMN2_ | − | CT | 1 | ACCAGCTATAAAGTAAAA | 3 | ACCAGCUAUAAAGUAAAA |
| exon6 | | TG | 7 | CTTATCGTTAAA | 8 | CUUAUCGUUAAA |
| | | | 1 | | 9 | |
| STMN2_ | − | CT | 1 | TAGTTCAGAAGAAATAAAC | 3 | UAGUUCAGAAGAAAUAAA |
| exon6 | | TT | 7 | TTGACCAGCTA | 9 | CUUGACCAGCUA |
| | | | 2 | | 0 | |
| STMN2_ | + | CT | 1 | AGATTAATAGAGTTTAACG | 3 | AGAUUAAUAGAGUUUAAC |
| exon6 | | TG | 7 | ATAAGTTTTAC | 9 | GAUAAGUUUUAC |
| | | | 3 | | 1 | |
| STMN2_ | + | AT | 1 | ATAGAGTTTAACGATAAGT | 3 | AUAGAGUUUAACGAUAAG |
| exon6 | | TA | 7 | TTTACTTTATA | 9 | UUUUACUUUAUA |
| | | | 4 | | 2 | |
| STMN2_ | + | GT | 1 | AACGATAAGTTTTACTTTA | 3 | AACGAUAAGUUUUACUUU |
| exon6 | | TT | 7 | TAGCTGGTCAA | 9 | AUAGCUGGUCAA |
| | | | 5 | | 3 | |
| STMN2_ | + | TT | 1 | ACGATAAGTTTTACTTTAT | 3 | ACGAUAAGUUUUACUUUA |
| exon6 | | TA | 7 | AGCTGGTCAAG | 9 | UAGCUGGUCAAG |
| | | | 6 | | 4 | |
| STMN2_ | + | GT | 1 | TACTTTATAGCTGGTCAAG | 3 | UACUUUAUAGCUGGUCAA |
| exon6 | | TT | 7 | TTTATTTCTTC | 9 | GUUUAUUUCUUC |
| | | | 7 | | 5 | |
| STMN2_ | + | TT | 1 | ACTTTATAGCTGGTCAAGT | 3 | ACUUUAUAGCUGGUCAAG |
| exon6 | | TT | 7 | TTATTTCTTCT | 9 | UUUAUUUCUUCU |
| | | | 8 | | 6 | |
| STMN2_ | + | TT | 1 | CTTTATAGCTGGTCAAGTT | 3 | CUUUAUAGCUGGUCAAGU |
| exon6 | | TA | 7 | TATTTCTTCTG | 9 | UUAUUUCUUCUG |
| | | | 9 | | 7 | |
| STMN2_ | + | CT | 1 | ATAGCTGGTCAAGTTTATT | 3 | AUAGCUGGUCAAGUUUAU |
| exon6 | | TT | 8 | TCTTCTGAACT | 9 | UUCUUCUGAACU |
| | | | 0 | | 8 | |
| STMN2_ | + | TT | 1 | TAGCTGGTCAAGTTTATTT | 3 | UAGCUGGUCAAGUUUAUU |
| exon6 | | TA | 8 | CTTCTGAACTA | 9 | UCUUCUGAACUA |
| | | | 1 | | 9 | |
| STMN2_ | + | GT | 1 | ATTTCTTCTGAACTAAAAG | 4 | AUUUCUUCUGAACUAAAA |
| exon6 | | TT | 8 | AATCTATAGAG | 0 | GAAUCUAUAGAG |
| | | | 2 | | 0 | |
| STMN2_ | + | TT | 1 | TTTCTTCTGAACTAAAAGA | 4 | UUUCUUCUGAACUAAAAG |
| exon6 | | TA | 8 | ATCTATAGAGT | 0 | AAUCUAUAGAGU |
| | | | 3 | | 1 | |
| STMN2_ | + | AT | 1 | CTTCTGAACTAAAAGAATC | 4 | CUUCUGAACUAAAAGAAU |
| exon6 | | TT | 8 | TATAGAGTCTC | 0 | CUAUAGAGUCUC |
| | | | 4 | | 2 | |
| STMN2_ | + | TT | 1 | TTCTGAACTAAAAGAATCT | 4 | UUCUGAACUAAAAGAAUC |
| exon6 | | TC | 8 | ATAGAGTCTCA | 0 | UAUAGAGUCUCA |
| | | | 5 | | 3 | |
| STMN2_ | + | CT | 1 | TGAACTAAAAGAATCTATA | 4 | UGAACUAAAAGAAUCUAU |
| exon6 | | TC | 8 | GAGTCTCAATT | 0 | AGAGUCUCAAUU |
| | | | 6 | | 4 | |
| STMN2_ | + | AT | 1 | CTGGAGCTTCAGAGGGAA | 4 | CUGGAGCUUCAGAGGGAA |
| exon6 | | TT | 8 | GGAGAGAAGCAA | 0 | GGAGAGAAGCAA |
| | | | 7 | | 5 | |
| STMN2_ | + | TT | 1 | TGGAGCTTCAGAGGGAAG | 4 | UGGAGCUUCAGAGGGAA |
| exon6 | | TC | 8 | GAGAGAAGCAAT | 0 | GGAGAGAAGCAAU |
| | | | 8 | | 6 | |
| STMN2_ | + | CT | 1 | AGAGGGAAGGAGAGAAG | 4 | AGAGGGAAGGAGAGAAGC |
| exon6 | | TC | 8 | CAATGTAAGCAAC | 0 | AAUGUAAGCAAC |
| | | | 9 | | 7 | |
| STMN2_ | − | AT | 1 | TTTTAGTTCAGAAGAAATA | 4 | UUUUAGUUCAGAAGAAAU |
| exon6 | | TC | 9 | AACTTGACCAG | 0 | AAACUUGACCAG |
| | | | 0 | | 8 | |
| STMN2_ | − | AT | 1 | AGACTCTATAGATTCTTTT | 4 | AGACUCUAUAGAUUCUUU |
| exon6 | | TG | 9 | AGTTCAGAAGA | 0 | UAGUUCAGAAGA |
| | | | 1 | | 9 | |
| STMN2_ | − | CT | 1 | CCTCTGAAGCTCCAGAAA | 4 | CCUCUGAAGCUCCAGAAA |
| exon6 | | TC | 9 | TTGAGACTCTAT | 1 | UUGAGACUCUAU |
| | | | 2 | | 0 | |
| STMN2_ | − | CT | 1 | TCTCCTTCCCTCTGAAGC | 4 | UCUCCUUCCCUCUGAAGC |
| exon6 | | TC | 9 | TCCAGAAATTGA | 1 | UCCAGAAAUUGA |
| | | | 3 | | 1 | |
| STMN2_ | − | AT | 1 | CTTCTCTCCTTCCCTCTGA | 4 | CUUCUCUCCUUCCCUCUG |
| exon6 | | TG | 9 | AGCTCCAGAAA | 1 | AAGCUCCAGAAA |
| | | | 4 | | 2 | |
| STMN2_ | − | CT | 1 | CATTGCTTCTCTCCTTCCC | 4 | CAUUGCUUCUCUCCUUCC |
| exon6 | | TA | 9 | TCTGAAGCTCC | 1 | CUCUGAAGCUCC |
| | | | 5 | | 3 | |
| STMN2_ | − | TT | 1 | AGTTCAGAAGAAATAAACT | 4 | AGUUCAGAAGAAAUAAAC |
| exon6 | | TT | 9 | TGACCAGCTAT | 1 | UUGACCAGCUAU |
| | | | 6 | | 4 | |
| STMN2_ | − | TT | 1 | TGTAGAATGTTGCTTACAT | 4 | UGUAGAAUGUUGCUUACA |
| exon6 | | TC | 9 | TGCTTCTCTCC | 1 | UUGCUUCUCUCC |
| | | | 7 | | 5 | |
| STMN2_ | − | TT | 1 | TATTTCTGTAGAATGTTGC | 4 | UAUUUCUGUAGAAUGUUG |
| exon6 | | TA | 9 | TTACATTGCTT | 1 | CUUACAUUGCUU |
| | | | 8 | | 6 | |
| STMN2_ | − | AT | 1 | ATATTTCTGTAGAATGTTG | 4 | AUAUUUCUGUAGAAUGUU |
| exon6 | | TT | 9 | CTTACATTGCT | 1 | GCUUACAUUGCU |
| | | | 9 | | 7 | |
| STMN2_ | − | AT | 2 | TTTATATTTCTGTAGAATG | 4 | UUUAUAUUUCUGUAGAAU |
| exon6 | | TA | 0 | TTGCTTACATT | 1 | GUUGCUUACAUU |
| | | | 0 | | 8 | |
| STMN2_ | − | AT | 2 | GTAGTATTATTTATATTTC | 4 | GUAGUAUUAUUUAUAUUU |
| exon6 | | TA | 0 | TGTAGAATGTT | 1 | CUGUAGAAUGUU |
| | | | 1 | | 9 | |
| STMN2_ | − | AT | 2 | TTAGTAGTATTATTTATATT | 4 | UUAGUAGUAUUAUUUAUA |
| exon6 | | TA | 0 | TCTGTAGAAT | 2 | UUUCUGUAGAAU |
| | | | 2 | | 0 | |
| STMN2_ | + | AT | 2 | TACAGAAATATAAATAATA | 4 | UACAGAAAUAUAAAUAAUA |
| exon6 | | TC | 0 | CTACTAATAAT | 2 | CUACUAAUAAU |
| | | | 3 | | 1 | |
| STMN2_ | | AT | 2 | CTGTAGAATGTTGCTTACA | 4 | CUGUAGAAUGUUGCUUAC |
| exon6 | | TT | 0 | TTGCTTCTCTC | 2 | AUUGCUUCUCUC |
| | | | 4 | | 2 | |
| STMN2_ | − | GT | 2 | CTTACATTGCTTCTCTCCT | 4 | CUUACAUUGCUUCUCUCC |
| exon6 | | TG | 0 | TCCCTCTGAAG | 2 | UUCCCUCUGAAG |
| | | | 5 | | 3 | |
| STMN2_ | − | GT | 2 | AACTCTATTAATCTCAAGG | 4 | AACUCUAUUAAUCUCAAG |
| exon6 | | TA | 0 | AGTCTACA | 2 | GAGUCUACA |
| | | | 6 | | 4 | |
| STMN2_ | + | TT | 2 | GTGTTTTTTAGGAGAGGC | 4 | GUGUUUUUUAGGAGAGG |
| exon7 | | TT | 0 | ATGCTGCGGAGG | 2 | CAUGCUGCGGAGG |
| | | | 7 | | 5 | |
| STMN2_ | + | TT | 2 | TTCTTCCTTTTGTGTTTTTT | 4 | UUCUUCCUUUUGUGUUUU |
| exon7 | | TC | 0 | AGGAGAGGCA | 2 | UUAGGAGAGGCA |
| | | | 8 | | 6 | |
| STMN2_ | + | TT | 2 | TGTTTTTTAGGAGAGGCA | 4 | UGUUUUUUAGGAGAGGCA |
| exon7 | | TG | 0 | TGCTGCGGAGGT | 2 | UGCUGCGGAGGU |
| | | | 9 | | 7 | |
| STMN2_ | + | GT | 2 | TTTAGGAGAGGCATGCTG | 4 | UUUAGGAGAGGCAUGCU |
| exon7 | | TT | 1 | CGGAGGTGCGCA | 2 | GCGGAGGUGCGCA |
| | | | 0 | | 8 | |
| STMN2_ | + | CT | 2 | CTTTTGTGTTTTTTAGGAG | 4 | CUUUUGUGUUUUUUAGGA |
| exon7 | | TC | 1 | AGGCATGCTGC | 2 | GAGGCAUGCUGC |
| | | | 1 | | 9 | |
| STMN2_ | + | TT | 2 | TAGGAGAGGCATGCTGCG | 4 | UAGGAGAGGCAUGCUGC |
| exon7 | | TT | 1 | GAGGTGCGCAGG | 3 | GGAGGUGCGCAGG |
| | | | 2 | | 0 | |
| STMN2_ | + | TT | 2 | AGGAGAGGCATGCTGCG | 4 | AGGAGAGGCAUGCUGCG |
| exon7 | | TT | 1 | GAGGTGCGCAGGA | 3 | GAGGUGCGCAGGA |
| | | | 3 | | 1 | |
| STMN2_ | + | TT | 2 | GGAGAGGCATGCTGCGG | 4 | GGAGAGGCAUGCUGCGG |
| exon7 | | TA | 1 | AGGTGCGCAGGAA | 3 | AGGUGCGCAGGAA |
| | | | 4 | | 2 | |
| STMN2_ | + | GT | 2 | AACTGTCTGGCTGAAGCA | 4 | AACUGUCUGGCUGAAGCA |
| exon7 | | TG | 1 | AGGGAGGGTCTG | 3 | AGGGAGGGUCUG |
| | | | 5 | | 3 | |
| STMN2_ | − | AT | 2 | ACTATTGGTGGGGCGTGC | 4 | ACUAUUGGUGGGGCGUG |
| exon7 | | TT | 1 | CAGACCCTCCCT | 3 | CCAGACCCUCCCU |
| | | | 6 | | 4 | |
| STMN2_ | + | AT | 2 | CTTCTTCCTTTTGTGTTTT | 4 | CUUCUUCCUUUUGUGUUU |
| exon7 | | TT | 1 | TTAGGAGAGGC | 3 | UUUAGGAGAGGC |
| | | | 7 | | 5 | |
| STMN2_ | − | TT | 2 | CTATTGGTGGGGCGTGCC | 4 | CUAUUGGUGGGGCGUGC |
| exon7 | | TA | 1 | AGACCCTCCCTT | 3 | CAGACCCUCCCUU |
| | | | 8 | | 6 | |
| STMN2_ | − | CT | 2 | CTTCAGCCAGACAGTTCA | 4 | CUUCAGCCAGACAGUUCA |
| exon7 | | TG | 1 | ACCTGGAGTTCC | 3 | ACCUGGAGUUCC |
| | | | 9 | | 7 | |
| STMN2_ | − | CT | 2 | AGCCAGACAGTTCAACCT | 4 | AGCCAGACAGUUCAACCU |
| exon7 | | TC | 2 | GGAGTTCCTTGT | 3 | GGAGUUCCUUGU |
| | | | 0 | | 8 | |
| STMN2_ | − | GT | 2 | AACCTGGAGTTCCTTGTT | 4 | AACCUGGAGUUCCUUGUU |
| exon7 | | TC | 2 | CCTGCGCACCTC | 3 | CCUGCGCACCUC |
| | | | 1 | | 9 | |
| STMN2_ | − | GT | 2 | CTTGTTCCTGCGCACCTC | 4 | CUUGUUCCUGCGCACCUC |
| exon7 | | TC | 2 | CGCAGCATGCCT | 4 | CGCAGCAUGCCU |
| | | | 2 | | 0 | |
| STMN2_ | − | CT | 2 | TTCCTGCGCACCTCCGCA | 4 | UUCCUGCGCACCUCCGCA |
| exon7 | | TG | 2 | GCATGCCTCTCC | 4 | GCAUGCCUCUCC |
| | | | 3 | | 1 | |
| STMN2_ | − | GT | 2 | CTGCGCACCTCCGCAGCA | 4 | CUGCGCACCUCCGCAGCA |
| exon7 | | TC | 2 | TGCCTCTCCTAA | 4 | UGCCUCUCCUAA |
| | | | 4 | | 2 | |
| STMN2_ | + | CT | 2 | TGTGTTTTTTAGGAGAGG | 4 | UGUGUUUUUUAGGAGAG |
| exon7 | | TT | 2 | CATGCTGCGGAG | 4 | GCAUGCUGCGGAG |
| | | | 5 | | 3 | |
| STMN2_ | + | CT | 2 | TTCCTTTTGTGTTTTTTAG | 4 | UUCCUUUUGUGUUUUUUA |
| exon7 | | TC | 2 | GAGAGGCATGC | 4 | GGAGAGGCAUGC |
| | | | 6 | | 4 | |
| STMN2_ | − | AT | 2 | GTGGGGCGTGCCAGACC | 4 | GUGGGGCGUGCCAGACC |
| exon7 | | TG | 2 | CTCCCTTGCTTCA | 4 | CUCCCUUGCUUCA |
| | | | 7 | | 5 | |
| STMN2_ | + | TT | 2 | TTAGGAGAGGCATGCTGC | 4 | UUAGGAGAGGCAUGCUG |
| exon7 | | TT | 2 | GGAGGTGCGCAG | 4 | CGGAGGUGCGCAG |
| | | | 8 | | 6 | |

TABLE 5B
Target and Spacer Sequences-intron

| refid | strand | PAM | | target | | spacer |
|---|---|---|---|---|---|---|
| STMN2_ | + | GTTC | 491 | TCCGTCGGCTCTACC | 2497 | UCCGUCGGCUCUACC |
| intron1 | | | | TGGAGCCCACCTCTC | | UGGAGCCCACCUCUC |
| STMN2_ | − | ATTT | 492 | GGAAGTATTTTCTCT | 2498 | GGAAGUAUUUUCUCU |
| intron1 | | | | TCAAGGTGAGTCTGT | | UCAAGGUGAGUCUGU |
| STMN2_ | − | ATTA | 493 | AAACTAGGCATCAAT | 2499 | AAACUAGGCAUCAAU |
| intron1 | | | | TTGGAAGTATTTTCT | | UUGGAAGUAUUUUCU |
| STMN2_ | − | TTTG | 494 | AATAAGCCCCAGGTA | 2500 | AAUAAGCCCCAGGUA |
| intron1 | | | | AGCTATTAAAACTAG | | AGCUAUUAAAACUAG |
| STMN2_ | − | ATTT | 495 | GAATAAGCCCCAGGT | 2501 | GAAUAAGCCCCAGGU |
| intron1 | | | | AAGCTATTAAAACTA | | AAGCUAUUAAAACUA |
| STMN2_ | − | ATTA | 496 | TTTGAATAAGCCCCA | 2502 | UUUGAAUAAGCCCCA |
| intron1 | | | | GGTAAGCTATTAAAA | | GGUAAGCUAUUAAAA |
| STMN2_ | − | TTTC | 497 | TCCCAAAGCCTAAAT | 2503 | UCCCAAAGCCUAAAU |
| intron1 | | | | CATGGCAATTATTTG | | CAUGGCAAUUAUUUG |
| STMN2_ | − | CTTT | 498 | CTCCCAAAGCCTAAA | 2504 | CUCCCAAAGCCUAAA |
| intron1 | | | | TCATGGCAATTATTT | | UCAUGGCAAUUAUUU |
| STMN2_ | − | GTTA | 499 | CAACCCACACGGCCT | 2505 | CAACCCACACGGCCU |
| intron1 | | | | CATAGCTCTCTTTCT | | CAUAGCUCUCUUUCU |
| STMN2_ | − | GTTC | 500 | CCACCAGAAATCGAT | 2506 | CCACCAGAAAUCGAU |
| intron1 | | | | GCTGTGCTGAGCCTG | | GCUGUGCUGAGCCUG |
| STMN2_ | − | TTTC | 501 | TGGAACTGGTCATCA | 2507 | UGGAACUGGUCAUCA |
| intron1 | | | | GAGTGTGTTCCCACC | | GAGUGUGUUCCCACC |
| STMN2_ | − | ATTT | 502 | CTGGAACTGGTCATC | 2508 | CUGGAACUGGUCAUC |
| intron1 | | | | AGAGTGTGTTCCCAC | | AGAGUGUGUUCCCAC |
| STMN2_ | − | GTTA | 503 | TTTCTGGAACTGGTC | 2509 | UUUCUGGAACUGGUC |
| intron1 | | | | ATCAGAGTGTGTTCC | | AUCAGAGUGUGUUCC |
| STMN2_ | − | ATTA | 504 | AGTCAATGTTATTTC | 2510 | AGUCAAUGUUAUUUC |
| intron1 | | | | TGGAACTGGTCATCA | | UGGAACUGGUCAUCA |
| STMN2_ | − | TTTG | 505 | AAATGTGCTAACCAT | 2511 | AAAUGUGCUAACCAU |
| intron1 | | | | GATGGGACTGAGGAG | | GAUGGGACUGAGGAG |
| STMN2_ | − | TTTT | 506 | GAAATGTGCTAACCA | 2512 | GAAAUGUGCUAACCA |
| intron1 | | | | TGATGGGACTGAGGA | | UGAUGGGACUGAGGA |
| STMN2_ | − | ATTT | 507 | TGAAATGTGCTAACC | 2513 | UGAAAUGUGCUAACC |
| intron1 | | | | ATGATGGGACTGAGG | | AUGAUGGGACUGAGG |
| STMN2_ | − | GTTA | 508 | AGGAGGCATTTTGAA | 2514 | AGGAGGCAUUUUGAA |
| intron1 | | | | ATGTGCTAACCATGA | | AUGUGCUAACCAUGA |
| STMN2_ | − | GTTA | 509 | AAACTAAATATCTCT | 2515 | AAACUAAAUAUCUCU |
| intron1 | | | | GGCCTATGGAAGTAG | | GGCCUAUGGAAGUAG |
| STMN2_ | − | ATTC | 510 | AACAAAATGTTAAAA | 2516 | AACAAAAUGUUAAAA |
| intron1 | | | | CTAAATATCTCTGGC | | CUAAAUAUCUCUGGC |
| STMN2_ | − | TTTA | 511 | TTCAACAAAATGTTA | 2517 | UUCAACAAAAUGUUA |
| intron1 | | | | AAACTAAATATCTCT | | AAACUAAAUAUCUCU |
| STMN2_ | − | TTTT | 512 | ATTCAACAAAATGTT | 2518 | AUUCAACAAAAUGUU |
| intron1 | | | | AAAACTAAATATCTC | | AAAACUAAAUAUCUC |
| STMN2_ | − | ATTT | 513 | TATTCAACAAAATGT | 2519 | UAUUCAACAAAAUGU |
| intron1 | | | | TAAAACTAAATATCT | | UAAAACUAAAUAUCU |
| STMN2_ | − | TTTA | 514 | TTTTATTCAACAAAA | 2520 | UUUUAUUCAACAAAA |
| intron1 | | | | TGTTAAAACTAAATA | | UGUUAAAACUAAAUA |
| STMN2_ | − | ATTT | 515 | ATTTTATTCAACAAA | 2521 | AUUUUAUUCAACAAA |
| intron1 | | | | ATGTTAAAACTAAAT | | AUGUUAAAACUAAAU |
| STMN2_ | − | ATTA | 516 | AATGTGAATGTGTAA | 2522 | AAUGUGAAUGUGUAA |
| intron1 | | | | ATTTATTTTATTCAA | | AUUUAUUUUAUUCAA |
| STMN2_ | − | GTTA | 517 | TATTAAATGTGAATG | 2523 | UAUUAAAUGUGAAUG |
| intron1 | | | | TGTAAATTTATTTTA | | UGUAAAUUUAUUUUA |
| STMN2_ | − | CTTG | 518 | AAATAACATCTAATA | 2524 | AAAUAACAUCUAAUA |
| intron1 | | | | GTTATATTAAATGTG | | GUUAUAUUAAAUGUG |
| STMN2_ | − | TTTG | 519 | GAAGTATTTTCTCTT | 2525 | GAAGUAUUUUCUCUU |
| intron1 | | | | CAAGGTGAGTCTGTG | | CAAGGUGAGUCUGUG |
| STMN2_ | − | TTTG | 520 | ATGGTAATATGAAGA | 2526 | AUGGUAAUAUGAAGA |
| intron1 | | | | GAATCTTGAAATAAC | | GAAUCUUGAAAUAAC |
| STMN2_ | − | ATTT | 521 | TCTCTTCAAGGTGAG | 2527 | UCUCUUCAAGGUGAG |
| intron1 | | | | TCTGTGATCAGAAAG | | UCUGUGAUCAGAAAG |
| STMN2_ | − | TTTC | 522 | TCTTCAAGGTGAGTC | 2528 | UCUUCAAGGUGAGUC |
| intron1 | | | | TGTGATCAGAAAGGA | | UGUGAUCAGAAAGGA |
| STMN2_ | − | ATTG | 523 | CGGGAAAATGTTTGA | 2529 | CGGGAAAAUGUUUGA |
| intron1 | | | | GTAAAGAAATAGGAA | | GUAAAGAAAUAGGAA |
| STMN2_ | − | GTTG | 524 | AAAGAAAGCACCATT | 2530 | AAAGAAAGCACCAUU |
| intron1 | | | | GCGGGAAAATGTTTG | | GCGGGAAAAUGUUUG |
| STMN2_ | − | TTTA | 525 | TGAATACACCAGAAA | 2531 | UGAAUACACCAGAAA |
| intron1 | | | | AACAGTTGAAAGAAA | | AACAGUUGAAAGAAA |
| STMN2_ | − | ATTT | 526 | ATGAATACACCAGAA | 2532 | AUGAAUACACCAGAA |
| intron1 | | | | AAACAGTTGAAAGAA | | AAACAGUUGAAAGAA |
| STMN2_ | − | CTTC | 527 | CCATAGAGAATCTGG | 2533 | CCAUAGAGAAUCUGG |
| intron1 | | | | AATTTATGAATACAC | | AAUUUAUGAAUACAC |
| STMN2_ | − | GTTA | 528 | CTTCCCATAGAGAAT | 2534 | CUUCCCAUAGAGAAU |
| intron1 | | | | CTGGAATTTATGAAT | | CUGGAAUUUAUGAAU |
| STMN2_ | − | GTTA | 529 | AATCAATCAATAAAA | 2535 | AAUCAAUCAAUAAAA |
| intron1 | | | | GTTACTTCCCATAGA | | GUUACUUCCCAUAGA |
| STMN2_ | − | GTTA | 530 | TATGTGCTATACAAG | 2536 | UAUGUGCUAUACAAG |
| intron1 | | | | GGTTAAATCAATCAA | | GGUUAAAUCAAUCAA |
| STMN2_ | − | CTTG | 531 | CATGTTATATGTGCT | 2537 | CAUGUUAUAUGUGCU |
| intron1 | | | | ATACAAGGGTTAAAT | | AUACAAGGGUUAAAU |
| STMN2_ | − | CTTA | 532 | GAACAATGCCTTGCA | 2538 | GAACAAUGCCUUGCA |
| intron1 | | | | TGTTATATGTGCTAT | | UGUUAUAUGUGCUAU |
| STMN2_ | − | GTTC | 533 | TTAGAACAATGCCTT | 2539 | UUAGAACAAUGCCUU |
| intron1 | | | | GCATGTTATATGTGC | | GCAUGUUAUAUGUGC |
| STMN2_ | − | GTTA | 534 | ATATGTGGAAAGTTC | 2540 | AUAUGUGGAAAGUUC |
| intron1 | | | | TTAGAACAATGCCTT | | UUAGAACAAUGCCUU |
| STMN2_ | − | ATTA | 535 | ACACAGTTAATATGT | 2541 | ACACAGUUAAUAUGU |
| intron1 | | | | GGAAAGTTCTTAGAA | | GGAAAGUUCUUAGAA |
| STMN2_ | − | ATTA | 536 | AGTGATTAACACAGT | 2542 | AGUGAUUAACACAGU |
| intron1 | | | | TAATATGTGGAAAGT | | UAAUAUGUGGAAAGU |
| STMN2_ | − | ATTA | 537 | TTAAGTGATTAACAC | 2543 | UUAAGUGAUUAACAC |
| intron1 | | | | AGTTAATATGTGGAA | | AGUUAAUAUGUGGAA |
| STMN2_ | − | CTTA | 538 | GGATTATTAAGTGAT | 2544 | GGAUUAUUAAGUGAU |
| intron1 | | | | TAACACAGTTAATAT | | UAACACAGUUAAUAU |
| STMN2_ | − | TTTC | 539 | CATATCTGTAATAGA | 2545 | CAUAUCUGUAAUAGA |
| intron1 | | | | ACCTACTTAGGATTA | | ACCUACUUAGGAUUA |
| STMN2_ | − | GTTT | 540 | CCATATCTGTAATAG | 2546 | CCAUAUCUGUAAUAG |
| intron1 | | | | AACCTACTTAGGATT | | AACCUACUUAGGAUU |
| STMN2_ | − | TTTC | 541 | TGTGCCTCAGTTTCC | 2547 | UGUGCCUCAGUUUCC |
| intron1 | | | | ATATCTGTAATAGAA | | AUAUCUGUAAUAGAA |
| STMN2_ | − | CTTT | 542 | CTGTGCCTCAGTTTC | 2548 | CUGUGCCUCAGUUUC |
| intron1 | | | | CATATCTGTAATAGA | | CAUAUCUGUAAUAGA |
| STMN2_ | − | CTTC | 543 | AACTTTCTGTGCCTC | 2549 | AACUUUCUGUGCCUC |
| intron1 | | | | AGTTTCCATATCTGT | | AGUUUCCAUAUCUGU |
| STMN2_ | − | CTTG | 544 | AGTAAGATACTTCAA | 2550 | AGUAAGAUACUUCAA |
| intron1 | | | | CTTTCTGTGCCTCAG | | CUUUCUGUGCCUCAG |
| STMN2_ | − | ATTC | 545 | TGGATCTGACTAACT | 2551 | UGGAUCUGACUAACU |
| intron1 | | | | GTGTGACCTTGAGTA | | GUGUGACCUUGAGUA |
| STMN2_ | − | ATTC | 546 | CGAAGCCAGATGGCC | 2552 | CGAAGCCAGAUGGCC |
| intron1 | | | | TGGGCCCAAATTCTG | | UGGGCCCAAAUUCUG |
| STMN2_ | − | TTTA | 547 | AATAAAATGGTGATA | 2553 | AAUAAAAUGGUGAUA |
| intron1 | | | | TCACAGGTGTGACCT | | UCACAGGUGUGACCU |
| STMN2_ | − | GTTT | 548 | AAATAAAATGGTGAT | 2554 | AAAUAAAAUGGUGAU |
| intron1 | | | | ATCACAGGTGTGACC | | AUCACAGGUGUGACC |
| STMN2_ | − | CTTC | 549 | AAGGTGAGTCTGTGA | 2555 | AAGGUGAGUCUGUGA |
| intron1 | | | | TCAGAAAGGAGAAGA | | UCAGAAAGGAGAAGA |
| STMN2_ | − | TTTT | 550 | CTCTTCAAGGTGAGT | 2556 | CUCUUCAAGGUGAGU |
| intron1 | | | | CTGTGATCAGAAAGG | | CUGUGAUCAGAAAGG |
| STMN2_ | − | CTTT | 551 | GATGGTAATATGAAG | 2557 | GAUGGUAAUAUGAAG |
| intron1 | | | | AGAATCTTGAAATAA | | AGAAUCUUGAAAUAA |
| STMN2_ | − | GTTC | 552 | TCTCCTGCCTGCCTG | 2558 | UCUCCUGCCUGCCUG |
| intron1 | | | | CCTGCTTTGATGGTA | | CCUGCUUUGAUGGUA |
| STMN2_ | − | CTTC | 553 | CTACAGTTCTCTCCT | 2559 | CUACAGUUCUCUCCU |
| intron1 | | | | GCCTGCCTGCCTGCT | | GCCUGCCUGCCUGCU |
| STMN2_ | − | ATTT | 554 | TTGTTATGGTTTTAT | 2560 | UUGUUAUGGUUUUAU |
| intron1 | | | | AGTATAATATGTGGC | | AGUAUAAUAUGUGGC |
| STMN2_ | − | CTTA | 555 | AAATATTTTTGTTAT | 2561 | AAAUAUUUUUGUUAU |
| intron1 | | | | GGTTTTATAGTATAA | | GGUUUUAUAGUAUAA |
| STMN2_ | − | TTTA | 556 | CTCTGGAGGTCAACA | 2562 | CUCUGGAGGUCAACA |
| intron1 | | | | ACAAGTGAGAACAAA | | ACAAGUGAGAACAAA |
| STMN2_ | − | TTTT | 557 | ACTCTGGAGGTCAAC | 2563 | ACUCUGGAGGUCAAC |
| intron1 | | | | AACAAGTGAGAACAA | | AACAAGUGAGAACAA |
| STMN2_ | − | ATTT | 558 | TACTCTGGAGGTCAA | 2564 | UACUCUGGAGGUCAA |
| intron1 | | | | CAACAAGTGAGAACA | | CAACAAGUGAGAACA |
| STMN2_ | − | ATTA | 559 | AATATTTTACTCTGG | 2565 | AAUAUUUUACUCUGG |
| intron1 | | | | AGGTCAACAACAAGT | | AGGUCAACAACAAGU |
| STMN2_ | − | TTTC | 560 | CAGAGTATTAAATAT | 2566 | CAGAGUAUUAAAUAU |
| intron1 | | | | TTTACTCTGGAGGTC | | UUUACUCUGGAGGUC |
| STMN2_ | − | CTTT | 561 | CCAGAGTATTAAATA | 2567 | CCAGAGUAUUAAAUA |
| intron1 | | | | TTTTACTCTGGAGGT | | UUUUACUCUGGAGGU |
| STMN2_ | − | TTTG | 562 | AAACCCATAACTTTC | 2568 | AAACCCAUAACUUUC |
| intron1 | | | | CAGAGTATTAAATAT | | CAGAGUAUUAAAUAU |
| STMN2_ | − | TTTT | 563 | GAAACCCATAACTTT | 2569 | GAAACCCAUAACUUU |
| intron1 | | | | CCAGAGTATTAAATA | | CCAGAGUAUUAAAUA |
| STMN2_ | − | ATTT | 564 | TGAAACCCATAACTT | 2570 | UGAAACCCAUAACUU |
| intron1 | | | | TCCAGAGTATTAAAT | | UCCAGAGUAUUAAAU |
| STMN2_ | − | CTTG | 565 | CCATAAAATAAATTT | 2571 | CCAUAAAAUAAAUUU |
| intron1 | | | | TGAAACCCATAACTT | | UGAAACCCAUAACUU |
| STMN2_ | − | TTTC | 566 | TTGCCATAAAATAAA | 2572 | UUGCCAUAAAAUAAA |
| intron1 | | | | TTTTGAAACCCATAA | | UUUUGAAACCCAUAA |
| STMN2_ | − | ATTT | 567 | CTTGCCATAAAATAA | 2573 | CUUGCCAUAAAAUAA |
| intron1 | | | | ATTTTGAAACCCATA | | AUUUUGAAACCCAUA |
| STMN2_ | − | ATTA | 568 | TCTATTTCTTGCCAT | 2574 | UCUAUUUCUUGCCAU |
| intron1 | | | | AAAATAAATTTTGAA | | AAAAUAAAUUUUGAA |
| STMN2_ | − | TTTA | 569 | AATGTGCTCTATGAG | 2575 | AAUGUGCUCUAUGAG |
| intron1 | | | | AACTGTAATTATCTA | | AACUGUAAUUAUCUA |
| STMN2_ | − | TTTT | 570 | AAATGTGCTCTATGA | 2576 | AAAUGUGCUCUAUGA |
| intron1 | | | | GAACTGTAATTATCT | | GAACUGUAAUUAUCU |
| STMN2_ | − | ATTT | 571 | TAAATGTGCTCTATG | 2577 | UAAAUGUGCUCUAUG |
| intron1 | | | | AGAACTGTAATTATC | | AGAACUGUAAUUAUC |
| STMN2_ | − | ATTA | 572 | TTTTAAATGTGCTCT | 2578 | UUUUAAAUGUGCUCU |
| intron1 | | | | ATGAGAACTGTAATT | | AUGAGAACUGUAAUU |
| STMN2_ | − | TTTG | 573 | CCCTATAAAAATAAA | 2579 | CCCUAUAAAAAUAAA |
| intron1 | | | | TTATTTTAAATGTGC | | UUAUUUUAAAUGUGC |
| STMN2_ | − | TTTT | 574 | GCCCTATAAAAATAA | 2580 | GCCCUAUAAAAAUAA |
| intron1 | | | | ATTATTTTAAATGTG | | AUUAUUUUAAAUGUG |
| STMN2_ | − | TTTT | 575 | TGCCCTATAAAAATA | 2581 | UGCCCUAUAAAAAUA |
| intron1 | | | | AATTATTTTAAATGT | | AAUUAUUUUAAAUGU |
| STMN2_ | − | ATTT | 576 | TTGCCCTATAAAAAT | 2582 | UUGCCCUAUAAAAAU |
| intron1 | | | | AAATTATTTTAAATG | | AAAUUAUUUUAAAUG |
| STMN2_ | − | ATTC | 577 | AGTCCTAGGCAATAT | 2583 | AGUCCUAGGCAAUAU |
| intron1 | | | | TTTTGCCCTATAAAA | | UUUUGCCCUAUAAAA |
| STMN2_ | − | TTTG | 578 | TAAAAAAAAAAAAAT | 2584 | UAAAAAAAAAAAAAU |
| intron1 | | | | CATTCAGTCCTAGGC | | CAUUCAGUCCUAGGC |
| STMN2_ | − | CTTT | 579 | GTAAAAAAAAAAAAA | 2585 | GUAAAAAAAAAAAAA |
| intron1 | | | | TCATTCAGTCCTAGG | | UCAUUCAGUCCUAGG |
| STMN2_ | − | TTTA | 580 | CAATCTTTGTAAAAA | 2586 | CAAUCUUUGUAAAAA |
| intron1 | | | | AAAAAAAATCATTCA | | AAAAAAAAUCAUUCA |
| STMN2_ | − | TTTT | 581 | TGTTATGGTTTTATA | 2587 | UGUUAUGGUUUUAUA |
| intron1 | | | | GTATAATATGTGGCT | | GUAUAAUAUGUGGCU |
| STMN2_ | − | TTTT | 582 | GTTATGGTTTTATAG | 2588 | GUUAUGGUUUUAUAG |
| intron1 | | | | TATAATATGTGGCTC | | UAUAAUAUGUGGCUC |
| STMN2_ | − | TTTG | 583 | TTATGGTTTTATAGT | 2589 | UUAUGGUUUUAUAGU |
| intron1 | | | | ATAATATGTGGCTCC | | AUAAUAUGUGGCUCC |
| STMN2_ | − | GTTA | 584 | TGGTTTTATAGTATA | 2590 | UGGUUUUAUAGUAUA |
| intron1 | | | | ATATGTGGCTCCTAC | | AUAUGUGGCUCCUAC |
| STMN2_ | − | ATTC | 585 | AAAACCTTCCTACAG | 2591 | AAAACCUUCCUACAG |
| intron1 | | | | TTCTCTCCTGCCTGC | | UUCUCUCCUGCCUGC |
| STMN2_ | − | TTTC | 586 | ACAAGGGATTCAAAA | 2592 | ACAAGGGAUUCAAAA |
| intron1 | | | | CCTTCCTACAGTTCT | | CCUUCCUACAGUUCU |
| STMN2_ | − | GTTT | 587 | CACAAGGGATTCAAA | 2593 | CACAAGGGAUUCAAA |
| intron1 | | | | ACCTTCCTACAGTTC | | ACCUUCCUACAGUUC |
| STMN2_ | − | ATTA | 588 | AAAATGTTTCACAAG | 2594 | AAAAUGUUUCACAAG |
| intron1 | | | | GGATTCAAAACCTTC | | GGAUUCAAAACCUUC |
| STMN2_ | − | ATTA | 589 | AAAGATAATTAAAAA | 2595 | AAAGAUAAUUAAAAA |
| intron1 | | | | TGTTTCACAAGGGAT | | UGUUUCACAAGGGAU |
| STMN2_ | − | TTTA | 590 | TTAAAAGATAATTAA | 2596 | UUAAAAGAUAAUUAA |
| intron1 | | | | AAATGTTTCACAAGG | | AAAUGUUUCACAAGG |
| STMN2_ | − | CTTT | 591 | ATTAAAAGATAATTA | 2597 | AUUAAAAGAUAAUUA |
| intron1 | | | | AAAATGTTTCACAAG | | AAAAUGUUUCACAAG |
| STMN2_ | − | ATTC | 592 | CTTTATTAAAAGATA | 2598 | CUUUAUUAAAAGAUA |
| intron1 | | | | ATTAAAAATGTTTCA | | AUUAAAAAUGUUUCA |
| STMN2_ | − | CTTG | 593 | ACAAATGACAGGGCC | 2599 | ACAAAUGACAGGGCC |
| intron1 | | | | TGATTCCTTTATTAA | | UGAUUCCUUUAUUAA |
| STMN2_ | − | TTTA | 594 | CTACTGCAAATGTCT | 2600 | CUACUGCAAAUGUCU |
| intron1 | | | | CCTTGACAAATGACA | | CCUUGACAAAUGACA |
| STMN2_ | − | CTTT | 595 | ACTACTGCAAATGTC | 2601 | ACUACUGCAAAUGUC |
| intron1 | | | | TCCTTGACAAATGAC | | UCCUUGACAAAUGAC |
| STMN2_ | − | ATTA | 596 | TAAACACAAGCTTTA | 2602 | UAAACACAAGCUUUA |
| intron1 | | | | CTACTGCAAATGTCT | | CUACUGCAAAUGUCU |
| STMN2_ | − | TTTA | 597 | ATCATGACTAATAAA | 2603 | AUCAUGACUAAUAAA |
| intron1 | | | | AATGGATATTATAAA | | AAUGGAUAUUAUAAA |
| STMN2_ | − | GTTT | 598 | GAGTAAAGAAATAGG | 2604 | GAGUAAAGAAAUAGG |
| intron1 | | | | AAGACTTATTGGCTC | | AAGACUUAUUGGCUC |
| STMN2_ | − | CTTT | 599 | AATCATGACTAATAA | 2605 | AAUCAUGACUAAUAA |
| intron1 | | | | AAATGGATATTATAA | | AAAUGGAUAUUAUAA |
| STMN2_ | − | TTTG | 600 | TGAGAACAAATGTAC | 2606 | UGAGAACAAAUGUAC |
| intron1 | | | | ACAAATGTTATCTTT | | ACAAAUGUUAUCUUU |
| STMN2_ | − | TTTT | 601 | GTGAGAACAAATGTA | 2607 | GUGAGAACAAAUGUA |
| intron1 | | | | CACAAATGTTATCTT | | CACAAAUGUUAUCUU |
| STMN2_ | − | GTTT | 602 | TGTGAGAACAAATGT | 2608 | UGUGAGAACAAAUGU |
| intron1 | | | | ACACAAATGTTATCT | | ACACAAAUGUUAUCU |
| STMN2_ | − | TTTA | 603 | CACTCATATAAAAGT | 2609 | CACUCAUAUAAAAGU |
| intron1 | | | | GTTTTGTGAGAACAA | | GUUUUGUGAGAACAA |
| STMN2_ | − | CTTT | 604 | ACACTCATATAAAAG | 2610 | ACACUCAUAUAAAAG |
| intron1 | | | | TGTTTTGTGAGAACA | | UGUUUUGUGAGAACA |
| STMN2_ | − | ATTA | 605 | ACCTTTACACTCATA | 2611 | ACCUUUACACUCAUA |
| intron1 | | | | TAAAAGTGTTTTGTG | | UAAAAGUGUUUUGUG |
| STMN2_ | − | ATTA | 606 | ATTAACCTTTACACT | 2612 | AUUAACCUUUACACU |
| intron1 | | | | CATATAAAAGTGTTT | | CAUAUAAAAGUGUUU |
| STMN2_ | − | TTTC | 607 | CACATGACCAGCAAA | 2613 | CACAUGACCAGCAAA |
| intron1 | | | | ATGATGGCTGAAATG | | AUGAUGGCUGAAAUG |
| STMN2_ | − | ATTT | 608 | CCACATGACCAGCAA | 2614 | CCACAUGACCAGCAA |
| intron1 | | | | AATGATGGCTGAAAT | | AAUGAUGGCUGAAAU |
| STMN2_ | − | ATTC | 609 | CTAAAGAAGCTATAT | 2615 | CUAAAGAAGCUAUAU |
| intron1 | | | | TTCCACATGACCAGC | | UUCCACAUGACCAGC |
| STMN2_ | − | TTTA | 610 | TAGTATAATATGTGG | 2616 | UAGUAUAAUAUGUGG |
| intron1 | | | | CTCCTACTCTAAGTA | | CUCCUACUCUAAGUA |
| STMN2_ | − | TTTT | 611 | ATAGTATAATATGTG | 2617 | AUAGUAUAAUAUGUG |
| intron1 | | | | GCTCCTACTCTAAGT | | GCUCCUACUCUAAGU |
| STMN2_ | − | GTTT | 612 | TATAGTATAATATGT | 2618 | UAUAGUAUAAUAUGU |
| intron1 | | | | GGCTCCTACTCTAAG | | GGCUCCUACUCUAAG |
| STMN2_ | − | GTTA | 613 | TCTTTAATCATGACT | 2619 | UCUUUAAUCAUGACU |
| intron1 | | | | AATAAAAATGGATAT | | AAUAAAAAUGGAUAU |
| STMN2_ | − | TTTG | 614 | AGTAAAGAAATAGGA | 2620 | AGUAAAGAAAUAGGA |
| intron1 | | | | AGACTTATTGGCTCG | | AGACUUAUUGGCUCG |
| STMN2_ | − | CTTA | 615 | TTGGCTCGAGGCCCT | 2621 | UUGGCUCGAGGCCCU |
| intron1 | | | | CAAGTTTAGATTTTT | | CAAGUUUAGAUUUUU |
| STMN2_ | − | ATTG | 616 | GCTCGAGGCCCTCAA | 2622 | GCUCGAGGCCCUCAA |
| intron1 | | | | GTTTAGATTTTTGTC | | GUUUAGAUUUUUGUC |
| STMN2_ | − | TTTG | 617 | TTTTAATTTCTTCAG | 2623 | UUUUAAUUUCUUCAG |
| intron1 | | | | TATTGCTATTCATAA | | UAUUGCUAUUCAUAA |
| STMN2_ | − | TTTT | 618 | GTTTTAATTTCTTCA | 2624 | GUUUUAAUUUCUUCA |
| intron1 | | | | GTATTGCTATTCATA | | GUAUUGCUAUUCAUA |
| STMN2_ | − | CTTT | 619 | TGTTTTAATTTCTTC | 2625 | UGUUUUAAUUUCUUC |
| intron1 | | | | AGTATTGCTATTCAT | | AGUAUUGCUAUUCAU |
| STMN2_ | − | ATTG | 620 | AGACAGCAATCTTTT | 2626 | AGACAGCAAUCUUUU |
| intron1 | | | | GTTTTAATTTCTTCA | | GUUUUAAUUUCUUCA |
| STMN2_ | − | TTTG | 621 | GTAAATAATAAATAT | 2627 | GUAAAUAAUAAAUAU |
| intron1 | | | | AAGATATATTGAGAC | | AAGAUAUAUUGAGAC |
| STMN2_ | − | ATTT | 622 | GGTAAATAATAAATA | 2628 | GGUAAAUAAUAAAUA |
| intron1 | | | | TAAGATATATTGAGA | | UAAGAUAUAUUGAGA |
| STMN2_ | − | CTTA | 623 | GAATAATTTGGTAAA | 2629 | GAAUAAUUUGGUAAA |
| intron1 | | | | TAATAAATATAAGAT | | UAAUAAAUAUAAGAU |
| STMN2_ | − | ATTC | 624 | AGGAAGAAATACTCT | 2630 | AGGAAGAAAUACUCU |
| intron1 | | | | TAGAATAATTTGGTA | | UAGAAUAAUUUGGUA |
| STMN2_ | − | TTTC | 625 | TCACATGGTATTCAG | 2631 | UCACAUGGUAUUCAG |
| intron1 | | | | GAAGAAATACTCTTA | | GAAGAAAUACUCUUA |
| STMN2_ | − | TTTT | 626 | CTCACATGGTATTCA | 2632 | CUCACAUGGUAUUCA |
| intron1 | | | | GGAAGAAATACTCTT | | GGAAGAAAUACUCUU |
| STMN2_ | − | ATTT | 627 | TCTCACATGGTATTC | 2633 | UCUCACAUGGUAUUC |
| intron1 | | | | AGGAAGAAATACTCT | | AGGAAGAAAUACUCU |
| STMN2_ | − | CTTA | 628 | AGAATTTTCTCACAT | 2634 | AGAAUUUUCUCACAU |
| intron1 | | | | GGTATTCAGGAAGAA | | GGUAUUCAGGAAGAA |
| STMN2_ | − | ATTC | 629 | TTAAGAATTTTCTCA | 2635 | UUAAGAAUUUUCUCA |
| intron1 | | | | CATGGTATTCAGGAA | | CAUGGUAUUCAGGAA |
| STMN2_ | − | TTTC | 630 | AAATATACAGTCATA | 2636 | AAAUAUACAGUCAUA |
| intron1 | | | | CTCAATAAATTCTTA | | CUCAAUAAAUUCUUA |
| STMN2_ | − | TTTT | 631 | CAAATATACAGTCAT | 2637 | CAAAUAUACAGUCAU |
| intron1 | | | | ACTCAATAAATTCTT | | ACUCAAUAAAUUCUU |
| STMN2_ | − | CTTT | 632 | TCAAATATACAGTCA | 2638 | UCAAAUAUACAGUCA |
| intron1 | | | | TACTCAATAAATTCT | | UACUCAAUAAAUUCU |
| STMN2_ | − | CTTA | 633 | GATAAGCAGAAGAAA | 2639 | GAUAAGCAGAAGAAA |
| intron1 | | | | ACACTCTTTTCAAAT | | ACACUCUUUUCAAAU |
| STMN2_ | − | ATTG | 634 | GCTTAGATAAGCAGA | 2640 | GCUUAGAUAAGCAGA |
| intron1 | | | | AGAAAACACTCTTTT | | AGAAAACACUCUUUU |
| STMN2_ | − | TTTA | 635 | TTGGCTTAGATAAGC | 2641 | UUGGCUUAGAUAAGC |
| intron1 | | | | AGAAGAAAACACTCT | | AGAAGAAAACACUCU |
| STMN2_ | − | CTTT | 636 | ATTGGCTTAGATAAG | 2642 | AUUGGCUUAGAUAAG |
| intron1 | | | | CAGAAGAAAACACTC | | CAGAAGAAAACACUC |
| STMN2_ | − | ATTG | 637 | AATAATGAAGATCCT | 2643 | AAUAAUGAAGAUCCU |
| intron1 | | | | TTATTGGCTTAGATA | | UUAUUGGCUUAGAUA |
| STMN2_ | − | GTTA | 638 | GAATTGAATAATGAA | 2644 | GAAUUGAAUAAUGAA |
| intron1 | | | | GATCCTTTATTGGCT | | GAUCCUUUAUUGGCU |
| STMN2_ | − | CTTA | 639 | GAAAGTTAGAATTGA | 2645 | GAAAGUUAGAAUUGA |
| intron1 | | | | ATAATGAAGATCCTT | | AUAAUGAAGAUCCUU |
| STMN2_ | − | CTTC | 640 | CTTAGAAAGTTAGAA | 2646 | CUUAGAAAGUUAGAA |
| intron1 | | | | TTGAATAATGAAGAT | | UUGAAUAAUGAAGAU |
| STMN2_ | − | GTTG | 641 | ACTTCCTTAGAAAGT | 2647 | ACUUCCUUAGAAAGU |
| intron1 | | | | TAGAATTGAATAATG | | UAGAAUUGAAUAAUG |
| STMN2_ | − | TTTC | 642 | TGATCTGTAGGTTGA | 2648 | UGAUCUGUAGGUUGA |
| intron1 | | | | CTTCCTTAGAAAGTT | | CUUCCUUAGAAAGUU |
| STMN2_ | − | CTTT | 643 | CTGATCTGTAGGTTG | 2649 | CUGAUCUGUAGGUUG |
| intron1 | | | | ACTTCCTTAGAAAGT | | ACUUCCUUAGAAAGU |
| STMN2_ | − | GTTT | 644 | TAATTTCTTCAGTAT | 2650 | UAAUUUCUUCAGUAU |
| intron1 | | | | TGCTATTCATAAATG | | UGCUAUUCAUAAAUG |
| STMN2_ | − | TTTT | 645 | AATTTCTTCAGTATT | 2651 | AAUUUCUUCAGUAUU |
| intron1 | | | | GCTATTCATAAATGA | | GCUAUUCAUAAAUGA |
| STMN2_ | − | TTTA | 646 | ATTTCTTCAGTATTG | 2652 | AUUUCUUCAGUAUUG |
| intron1 | | | | CTATTCATAAATGAT | | CUAUUCAUAAAUGAU |
| STMN2_ | − | ATTT | 647 | CTTCAGTATTGCTAT | 2653 | CUUCAGUAUUGCUAU |
| intron1 | | | | TCATAAATGATAGTA | | UCAUAAAUGAUAGUA |
| STMN2_ | − | ATTA | 648 | AGAGAGAGTGATGGG | 2654 | AGAGAGAGUGAUGGG |
| intron1 | | | | GCAGAACACATAATT | | GCAGAACACAUAAUU |
| STMN2_ | − | TTTA | 649 | AAAATCCAATTAAGA | 2655 | AAAAUCCAAUUAAGA |
| intron1 | | | | GAGAGTGATGGGGCA | | GAGAGUGAUGGGGCA |
| STMN2_ | − | TTTT | 650 | AAAAATCCAATTAAG | 2656 | AAAAAUCCAAUUAAG |
| intron1 | | | | AGAGAGTGATGGGGC | | AGAGAGUGAUGGGGC |
| STMN2_ | − | ATTT | 651 | TAAAAATCCAATTAA | 2657 | UAAAAAUCCAAUUAA |
| intron1 | | | | GAGAGAGTGATGGGG | | GAGAGAGUGAUGGGG |
| STMN2_ | − | CTTC | 652 | TGCCGAGTCCTGCAA | 2658 | UGCCGAGUCCUGCAA |
| intron1 | | | | TATGAATATAATTTT | | UAUGAAUAUAAUUUU |
| STMN2_ | − | TTTC | 653 | TCTCGAAGGTCTTCT | 2659 | UCUCGAAGGUCUUCU |
| intron1 | | | | GCCGAGTCCTGCAAT | | GCCGAGUCCUGCAAU |
| STMN2_ | − | CTTT | 654 | CTCTCGAAGGTCTTC | 2660 | CUCUCGAAGGUCUUC |
| intron1 | | | | TGCCGAGTCCTGCAA | | UGCCGAGUCCUGCAA |
| STMN2_ | − | TTTC | 655 | TACCTTTCTCTCGAA | 2661 | UACCUUUCUCUCGAA |
| intron1 | | | | GGTCTTCTGCCGAGT | | GGUCUUCUGCCGAGU |
| STMN2_ | − | TTTT | 656 | CTACCTTTCTCTCGA | 2662 | CUACCUUUCUCUCGA |
| intron1 | | | | AGGTCTTCTGCCGAG | | AGGUCUUCUGCCGAG |
| STMN2_ | − | ATTT | 657 | TCTACCTTTCTCTCG | 2663 | UCUACCUUUCUCUCG |
| intron1 | | | | AAGGTCTTCTGCCGA | | AAGGUCUUCUGCCGA |
| STMN2_ | − | CTTA | 658 | TTTTCTACCTTTCTC | 2664 | UUUUCUACCUUUCUC |
| intron1 | | | | TCGAAGGTCTTCTGC | | UCGAAGGUCUUCUGC |
| STMN2_ | − | ATTC | 659 | TTATTTTCTACCTTT | 2665 | UUAUUUUCUACCUUU |
| intron1 | | | | CTCTCGAAGGTCTTC | | CUCUCGAAGGUCUUC |
| STMN2_ | − | CTTA | 660 | GGCAGGCTGTCTGTC | 2666 | GGCAGGCUGUCUGUC |
| intron1 | | | | TCTCTCTCTCGCACA | | UCUCUCUCUCGCACA |
| STMN2_ | − | CTTG | 661 | AAGATCCTCTTTCTG | 2667 | AAGAUCCUCUUUCUG |
| intron1 | | | | ATCTGTAGGTTGACT | | AUCUGUAGGUUGACU |
| STMN2_ | − | CTTC | 662 | TTAGGCAGGCTGTCT | 2668 | UUAGGCAGGCUGUCU |
| intron1 | | | | GTCTCTCTCTCTCGC | | GUCUCUCUCUCUCGC |
| STMN2_ | − | ATTT | 663 | CTTCTTAGGCAGGCT | 2669 | CUUCUUAGGCAGGCU |
| intron1 | | | | GTCTGTCTCTCTCTC | | GUCUGUCUCUCUCUC |
| STMN2_ | − | ATTC | 664 | ATTTCTTCTTAGGCA | 2670 | AUUUCUUCUUAGGCA |
| intron1 | | | | GGCTGTCTGTCTCTC | | GGCUGUCUGUCUCUC |
| STMN2_ | − | ATTC | 665 | ACATTCATTTCTTCT | 2671 | ACAUUCAUUUCUUCU |
| intron1 | | | | TAGGCAGGCTGTCTG | | UAGGCAGGCUGUCUG |
| STMN2_ | − | CTTG | 666 | TCAACTGTGCCACAA | 2672 | UCAACUGUGCCACAA |
| intron1 | | | | GCCGCATTCACATTC | | GCCGCAUUCACAUUC |
| STMN2_ | − | TTTA | 667 | TCATCCTTGTCAACT | 2673 | UCAUCCUUGUCAACU |
| intron1 | | | | GTGCCACAAGCCGCA | | GUGCCACAAGCCGCA |
| STMN2_ | − | ATTT | 668 | ATCATCCTTGTCAAC | 2674 | AUCAUCCUUGUCAAC |
| intron1 | | | | TGTGCCACAAGCCGC | | UGUGCCACAAGCCGC |
| STMN2_ | − | ATTG | 669 | ATTTATCATCCTTGT | 2675 | AUUUAUCAUCCUUGU |
| intron1 | | | | CAACTGTGCCACAAG | | CAACUGUGCCACAAG |
| STMN2_ | − | ATTA | 670 | TTGATTTATCATCCT | 2676 | UUGAUUUAUCAUCCU |
| intron1 | | | | TGTCAACTGTGCCAC | | UGUCAACUGUGCCAC |
| STMN2_ | − | CTTG | 671 | CATTATTGATTTATC | 2677 | CAUUAUUGAUUUAUC |
| intron1 | | | | ATCCTTGTCAACTGT | | AUCCUUGUCAACUGU |
| STMN2_ | − | ATTC | 672 | ATAAATGATAGTAAG | 2678 | AUAAAUGAUAGUAAG |
| intron1 | | | | CTTGCATTATTGATT | | CUUGCAUUAUUGAUU |
| STMN2_ | − | ATTG | 673 | CTATTCATAAATGAT | 2679 | CUAUUCAUAAAUGAU |
| intron1 | | | | AGTAAGCTTGCATTA | | AGUAAGCUUGCAUUA |
| STMN2_ | − | CTTC | 674 | AGTATTGCTATTCAT | 2680 | AGUAUUGCUAUUCAU |
| intron1 | | | | AAATGATAGTAAGCT | | AAAUGAUAGUAAGCU |
| STMN2_ | − | TTTC | 675 | TTCAGTATTGCTATT | 2681 | UUCAGUAUUGCUAUU |
| intron1 | | | | CATAAATGATAGTAA | | CAUAAAUGAUAGUAA |
| STMN2_ | − | TTTC | 676 | TTCTTAGGCAGGCTG | 2682 | UUCUUAGGCAGGCUG |
| intron1 | | | | TCTGTCTCTCTCTCT | | UCUGUCUCUCUCUCU |
| STMN2_ | − | CTTT | 677 | ACAATCTTTGTAAAA | 2683 | ACAAUCUUUGUAAAA |
| intron1 | | | | AAAAAAAAATCATTC | | AAAAAAAAAUCAUUC |
| STMN2_ | − | ATTC | 678 | CTTGAAGATCCTCTT | 2684 | CUUGAAGAUCCUCUU |
| intron1 | | | | TCTGATCTGTAGGTT | | UCUGAUCUGUAGGUU |
| STMN2_ | − | CTTT | 679 | GATGCTATTCCTTGA | 2685 | GAUGCUAUUCCUUGA |
| intron1 | | | | AGATCCTCTTTCTGA | | AGAUCCUCUUUCUGA |
| STMN2_ | − | CTTA | 680 | GTCCAACTTTGTGTT | 2686 | GUCCAACUUUGUGUU |
| intron1 | | | | GAGTAACAGTATATT | | GAGUAACAGUAUAUU |
| STMN2_ | − | TTTG | 681 | AGACTTAGTCCAACT | 2687 | AGACUUAGUCCAACU |
| intron1 | | | | TTGTGTTGAGTAACA | | UUGUGUUGAGUAACA |
| STMN2_ | − | CTTT | 682 | GAGACTTAGTCCAAC | 2688 | GAGACUUAGUCCAAC |
| intron1 | | | | TTTGTGTTGAGTAAC | | UUUGUGUUGAGUAAC |
| STMN2_ | − | GTTA | 683 | ACAACAACTGAATGG | 2689 | ACAACAACUGAAUGG |
| intron1 | | | | CTAACTTTGAGACTT | | CUAACUUUGAGACUU |
| STMN2_ | − | CTTC | 684 | TGAGAGACCCTGAAA | 2690 | UGAGAGACCCUGAAA |
| intron1 | | | | TGAACTGTTAACAAC | | UGAACUGUUAACAAC |
| STMN2_ | − | TTTC | 685 | CCAGCTTCTGAGAGA | 2691 | CCAGCUUCUGAGAGA |
| intron1 | | | | CCCTGAAATGAACTG | | CCCUGAAAUGAACUG |
| STMN2_ | − | GTTT | 686 | CCCAGCTTCTGAGAG | 2692 | CCCAGCUUCUGAGAG |
| intron1 | | | | ACCCTGAAATGAACT | | ACCCUGAAAUGAACU |
| STMN2_ | − | ATTG | 687 | CAAAAATGGAAAGTT | 2693 | CAAAAAUGGAAAGUU |
| intron1 | | | | TCCCAGCTTCTGAGA | | UCCCAGCUUCUGAGA |
| STMN2_ | − | CTTC | 688 | AATGTACAAGAAATT | 2694 | AAUGUACAAGAAAUU |
| intron1 | | | | GCAAAAATGGAAAGT | | GCAAAAAUGGAAAGU |
| STMN2_ | − | TTTC | 689 | CTTCAATGTACAAGA | 2695 | CUUCAAUGUACAAGA |
| intron1 | | | | AATTGCAAAAATGGA | | AAUUGCAAAAAUGGA |
| STMN2_ | − | CTTT | 690 | CCTTCAATGTACAAG | 2696 | CCUUCAAUGUACAAG |
| intron1 | | | | AAATTGCAAAAATGG | | AAAUUGCAAAAAUGG |
| STMN2_ | − | CTTC | 691 | CTTTCCTTCAATGTA | 2697 | CUUUCCUUCAAUGUA |
| intron1 | | | | CAAGAAATTGCAAAA | | CAAGAAAUUGCAAAA |
| STMN2_ | − | CTTA | 692 | AGTGTGTCTTCCTTT | 2698 | AGUGUGUCUUCCUUU |
| intron1 | | | | CCTTCAATGTACAAG | | CCUUCAAUGUACAAG |
| STMN2_ | − | TTTG | 693 | TAATGCTGTCTTAAG | 2699 | UAAUGCUGUCUUAAG |
| intron1 | | | | TGTGTCTTCCTTTCC | | UGUGUCUUCCUUUCC |
| STMN2_ | − | TTTT | 694 | GTAATGCTGTCTTAA | 2700 | GUAAUGCUGUCUUAA |
| intron1 | | | | GTGTGTCTTCCTTTC | | GUGUGUCUUCCUUUC |
| STMN2_ | − | CTTT | 695 | TGTAATGCTGTCTTA | 2701 | UGUAAUGCUGUCUUA |
| intron1 | | | | AGTGTGTCTTCCTTT | | AGUGUGUCUUCCUUU |
| STMN2_ | − | ATTA | 696 | CTTTTGTAATGCTGT | 2702 | CUUUUGUAAUGCUGU |
| intron1 | | | | CTTAAGTGTGTCTTC | | CUUAAGUGUGUCUUC |
| STMN2_ | − | TTTA | 697 | AAACATGAATTACTT | 2703 | AAACAUGAAUUACUU |
| intron1 | | | | TTGTAATGCTGTCTT | | UUGUAAUGCUGUCUU |
| STMN2_ | − | ATTT | 698 | AAAACATGAATTACT | 2704 | AAAACAUGAAUUACU |
| intron1 | | | | TTTGTAATGCTGTCT | | UUUGUAAUGCUGUCU |
| STMN2_ | − | ATTA | 699 | AACATTTAAAACATG | 2705 | AACAUUUAAAACAUG |
| intron1 | | | | AATTACTTTTGTAAT | | AAUUACUUUUGUAAU |
| STMN2_ | − | GTTA | 700 | TACAGAGAGCCCTGC | 2706 | UACAGAGAGCCCUGC |
| intron1 | | | | CCGACTGCCAGAATT | | CCGACUGCCAGAAUU |
| STMN2_ | − | TTTG | 701 | TCATCTCCAAATGAG | 2707 | UCAUCUCCAAAUGAG |
| intron1 | | | | GTTATACAGAGAGCC | | GUUAUACAGAGAGCC |
| STMN2_ | − | TTTT | 702 | GTCATCTCCAAATGA | 2708 | GUCAUCUCCAAAUGA |
| intron1 | | | | GGTTATACAGAGAGC | | GGUUAUACAGAGAGC |
| STMN2_ | − | TTTT | 703 | TGTCATCTCCAAATG | 2709 | UGUCAUCUCCAAAUG |
| intron1 | | | | AGGTTATACAGAGAG | | AGGUUAUACAGAGAG |
| STMN2_ | − | ATTT | 704 | TTGTCATCTCCAAAT | 2710 | UUGUCAUCUCCAAAU |
| intron1 | | | | GAGGTTATACAGAGA | | GAGGUUAUACAGAGA |
| STMN2_ | − | TTTA | 705 | GATTTTTGTCATCTC | 2711 | GAUUUUUGUCAUCUC |
| intron1 | | | | CAAATGAGGTTATAC | | CAAAUGAGGUUAUAC |
| STMN2_ | − | GTTT | 706 | AGATTTTTGTCATCT | 2712 | AGAUUUUUGUCAUCU |
| intron1 | | | | CCAAATGAGGTTATA | | CCAAAUGAGGUUAUA |
| STMN2_ | − | CTTT | 707 | GTGTTGAGTAACAGT | 2713 | GUGUUGAGUAACAGU |
| intron1 | | | | ATATTCTGCAAACCC | | AUAUUCUGCAAACCC |
| STMN2_ | − | TTTG | 708 | TGTTGAGTAACAGTA | 2714 | UGUUGAGUAACAGUA |
| intron1 | | | | TATTCTGCAAACCCT | | UAUUCUGCAAACCCU |
| STMN2_ | − | GTTG | 709 | AGTAACAGTATATTC | 2715 | AGUAACAGUAUAUUC |
| intron1 | | | | TGCAAACCCTGAAGC | | UGCAAACCCUGAAGC |
| STMN2_ | − | ATTC | 710 | TGCAAACCCTGAAGC | 2716 | UGCAAACCCUGAAGC |
| intron1 | | | | TAGTTTTATTTGGGA | | UAGUUUUAUUUGGGA |
| STMN2_ | − | TTTC | 711 | CAGAAAGGTGGTAAT | 2717 | CAGAAAGGUGGUAAU |
| intron1 | | | | GGCTGCATGGTCAGC | | GGCUGCAUGGUCAGC |
| STMN2_ | − | ATTT | 712 | CCAGAAAGGTGGTAA | 2718 | CCAGAAAGGUGGUAA |
| intron1 | | | | TGGCTGCATGGTCAG | | UGGCUGCAUGGUCAG |
| STMN2_ | − | TTTG | 713 | CAGCATAATATTTCC | 2719 | CAGCAUAAUAUUUCC |
| intron1 | | | | AGAAAGGTGGTAATG | | AGAAAGGUGGUAAUG |
| STMN2_ | − | TTTT | 714 | GCAGCATAATATTTC | 2720 | GCAGCAUAAUAUUUC |
| intron1 | | | | CAGAAAGGTGGTAAT | | CAGAAAGGUGGUAAU |
| STMN2_ | − | TTTT | 715 | TGCAGCATAATATTT | 2721 | UGCAGCAUAAUAUUU |
| intron1 | | | | CCAGAAAGGTGGTAA | | CCAGAAAGGUGGUAA |
| STMN2_ | − | ATTT | 716 | TTGCAGCATAATATT | 2722 | UUGCAGCAUAAUAUU |
| intron1 | | | | TCCAGAAAGGTGGTA | | UCCAGAAAGGUGGUA |
| STMN2_ | − | ATTG | 717 | TATCATTTTTGCAGC | 2723 | UAUCAUUUUUGCAGC |
| intron1 | | | | ATAATATTTCCAGAA | | AUAAUAUUUCCAGAA |
| STMN2_ | − | TTTC | 718 | GTGTATTGTATCATT | 2724 | GUGUAUUGUAUCAUU |
| intron1 | | | | TTTGCAGCATAATAT | | UUUGCAGCAUAAUAU |
| STMN2_ | − | ATTT | 719 | CGTGTATTGTATCAT | 2725 | CGUGUAUUGUAUCAU |
| intron1 | | | | TTTTGCAGCATAATA | | UUUUGCAGCAUAAUA |
| STMN2_ | − | TTTG | 720 | AGATATTTCGTGTAT | 2726 | AGAUAUUUCGUGUAU |
| intron1 | | | | TGTATCATTTTTGCA | | UGUAUCAUUUUUGCA |
| STMN2_ | − | ATTT | 721 | GAGATATTTCGTGTA | 2727 | GAGAUAUUUCGUGUA |
| intron1 | | | | TTGTATCATTTTTGC | | UUGUAUCAUUUUUGC |
| STMN2_ | − | TTTA | 722 | ATTTGAGATATTTCG | 2728 | AUUUGAGAUAUUUCG |
| intron1 | | | | TGTATTGTATCATTT | | UGUAUUGUAUCAUUU |
| STMN2_ | − | TTTT | 723 | AATTTGAGATATTTC | 2729 | AAUUUGAGAUAUUUC |
| intron1 | | | | GTGTATTGTATCATT | | GUGUAUUGUAUCAUU |
| STMN2_ | − | TTTG | 724 | ATGCTATTCCTTGAA | 2730 | AUGCUAUUCCUUGAA |
| intron1 | | | | GATCCTCTTTCTGAT | | GAUCCUCUUUCUGAU |
| STMN2_ | − | TTTT | 725 | TAATTTGAGATATTT | 2731 | UAAUUUGAGAUAUUU |
| intron1 | | | | CGTGTATTGTATCAT | | CGUGUAUUGUAUCAU |
| STMN2_ | − | ATTT | 726 | TTTAATTTGAGATAT | 2732 | UUUAAUUUGAGAUAU |
| intron1 | | | | TTCGTGTATTGTATC | | UUCGUGUAUUGUAUC |
| STMN2_ | − | GTTA | 727 | TATTTTTTAATTTGA | 2733 | UAUUUUUUAAUUUGA |
| intron1 | | | | GATATTTCGTGTATT | | GAUAUUUCGUGUAUU |
| STMN2_ | − | TTTG | 728 | GGAAATGTTATATTT | 2734 | GGAAAUGUUAUAUUU |
| intron1 | | | | TTTAATTTGAGATAT | | UUUAAUUUGAGAUAU |
| STMN2_ | − | ATTT | 729 | GGGAAATGTTATATT | 2735 | GGGAAAUGUUAUAUU |
| intron1 | | | | TTTTAATTTGAGATA | | UUUUAAUUUGAGAUA |
| STMN2_ | − | TTTA | 730 | GTGCCCTATTTGGGA | 2736 | GUGCCCUAUUUGGGA |
| intron1 | | | | AATGTTATATTTTTT | | AAUGUUAUAUUUUUU |
| STMN2_ | − | TTTT | 731 | AGTGCCCTATTTGGG | 2737 | AGUGCCCUAUUUGGG |
| intron1 | | | | AAATGTTATATTTTT | | AAAUGUUAUAUUUUU |
| STMN2_ | − | TTTT | 732 | TAGTGCCCTATTTGG | 2738 | UAGUGCCCUAUUUGG |
| intron1 | | | | GAAATGTTATATTTT | | GAAAUGUUAUAUUUU |
| STMN2_ | − | GTTT | 733 | TTAGTGCCCTATTTG | 2739 | UUAGUGCCCUAUUUG |
| intron1 | | | | GGAAATGTTATATTT | | GGAAAUGUUAUAUUU |
| STMN2_ | − | TTTG | 734 | GGATCATGTTTTTAG | 2740 | GGAUCAUGUUUUUAG |
| intron1 | | | | TGCCCTATTTGGGAA | | UGCCCUAUUUGGGAA |
| STMN2_ | − | ATTT | 735 | GGGATCATGTTTTTA | 2741 | GGGAUCAUGUUUUUA |
| intron1 | | | | GTGCCCTATTTGGGA | | GUGCCCUAUUUGGGA |
| STMN2_ | − | TTTA | 736 | TTTGGGATCATGTTT | 2742 | UUUGGGAUCAUGUUU |
| intron1 | | | | TTAGTGCCCTATTTG | | UUAGUGCCCUAUUUG |
| STMN2_ | − | TTTT | 737 | ATTTGGGATCATGTT | 2743 | AUUUGGGAUCAUGUU |
| intron1 | | | | TTTAGTGCCCTATTT | | UUUAGUGCCCUAUUU |
| STMN2_ | − | GTTT | 738 | TATTTGGGATCATGT | 2744 | UAUUUGGGAUCAUGU |
| intron1 | | | | TTTTAGTGCCCTATT | | UUUUAGUGCCCUAUU |
| STMN2_ | − | TTTT | 739 | TTAATTTGAGATATT | 2745 | UUAAUUUGAGAUAUU |
| intron1 | | | | TCGTGTATTGTATCA | | UCGUGUAUUGUAUCA |
| STMN2_ | − | ATTC | 740 | CCAGAGTAATAAAAT | 2746 | CCAGAGUAAUAAAAU |
| intron1 | | | | CCCCAGGTATATGAG | | CCCCAGGUAUAUGAG |
| STMN2_ | − | GTTG | 741 | CTTTACAATCTTTGT | 2747 | CUUUACAAUCUUUGU |
| intron1 | | | | AAAAAAAAAAAAATC | | AAAAAAAAAAAAAUC |
| STMN2_ | − | ATTC | 742 | CAGAAGAATAACTGC | 2748 | CAGAAGAAUAACUGC |
| intron1 | | | | TAAATGGGCACTCTT | | UAAAUGGGCACUCUU |
| STMN2_ | − | TTTT | 743 | TATTTTTGTTCTCAT | 2749 | UAUUUUUGUUCUCAU |
| intron1 | | | | AATACCTGGCACAGG | | AAUACCUGGCACAGG |
| STMN2_ | − | ATTT | 744 | TTATTTTTGTTCTCA | 2750 | UUAUUUUUGUUCUCA |
| intron1 | | | | TAATACCTGGCACAG | | UAAUACCUGGCACAG |
| STMN2_ | − | TTTC | 745 | TGCAAAAGACTAAAT | 2751 | UGCAAAAGACUAAAU |
| intron1 | | | | CCACCAAGGGTGAGG | | CCACCAAGGGUGAGG |
| STMN2_ | − | TTTT | 746 | CTGCAAAAGACTAAA | 2752 | CUGCAAAAGACUAAA |
| intron1 | | | | TCCACCAAGGGTGAG | | UCCACCAAGGGUGAG |
| STMN2_ | − | TTTT | 747 | TCTGCAAAAGACTAA | 2753 | UCUGCAAAAGACUAA |
| intron1 | | | | ATCCACCAAGGGTGA | | AUCCACCAAGGGUGA |
| STMN2_ | − | TTTT | 748 | TTCTGCAAAAGACTA | 2754 | UUCUGCAAAAGACUA |
| intron1 | | | | AATCCACCAAGGGTG | | AAUCCACCAAGGGUG |
| STMN2_ | − | TTTT | 749 | TTTCTGCAAAAGACT | 2755 | UUUCUGCAAAAGACU |
| intron1 | | | | AAATCCACCAAGGGT | | AAAUCCACCAAGGGU |
| STMN2_ | − | CTTT | 750 | TTTTCTGCAAAAGAC | 2756 | UUUUCUGCAAAAGAC |
| intron1 | | | | TAAATCCACCAAGGG | | UAAAUCCACCAAGGG |
| STMN2_ | − | TTTC | 751 | TGACATGTACAGGAT | 2757 | UGACAUGUACAGGAU |
| intron1 | | | | CTTTTTTTCTGCAAA | | CUUUUUUUCUGCAAA |
| STMN2_ | − | CTTT | 752 | CTGACATGTACAGGA | 2758 | CUGACAUGUACAGGA |
| intron1 | | | | TCTTTTTTTCTGCAA | | UCUUUUUUUCUGCAA |
| STMN2_ | − | ATTG | 753 | AACTTTCTGACATGT | 2759 | AACUUUCUGACAUGU |
| intron1 | | | | ACAGGATCTTTTTTT | | ACAGGAUCUUUUUUU |
| STMN2_ | − | ATTA | 754 | CTATTGAACTTTCTG | 2760 | CUAUUGAACUUUCUG |
| intron1 | | | | ACATGTACAGGATCT | | ACAUGUACAGGAUCU |
| STMN2_ | − | ATTA | 755 | TTACTATTGAACTTT | 2761 | UUACUAUUGAACUUU |
| intron1 | | | | CTGACATGTACAGGA | | CUGACAUGUACAGGA |
| STMN2_ | − | ATTA | 756 | CCATTATTACTATTG | 2762 | CCAUUAUUACUAUUG |
| intron1 | | | | AACTTTCTGACATGT | | AACUUUCUGACAUGU |
| STMN2_ | − | GTTA | 757 | TAAATTACCATTATT | 2763 | UAAAUUACCAUUAUU |
| intron1 | | | | ACTATTGAACTTTCT | | ACUAUUGAACUUUCU |
| STMN2_ | − | TTTA | 758 | TAGTTATAAATTACC | 2764 | UAGUUAUAAAUUACC |
| intron1 | | | | ATTATTACTATTGAA | | AUUAUUACUAUUGAA |
| STMN2_ | − | ATTT | 759 | ATAGTTATAAATTAC | 2765 | AUAGUUAUAAAUUAC |
| intron1 | | | | CATTATTACTATTGA | | CAUUAUUACUAUUGA |
| STMN2_ | − | CTTC | 760 | CATTTATAGTTATAA | 2766 | CAUUUAUAGUUAUAA |
| intron1 | | | | ATTACCATTATTACT | | AUUACCAUUAUUACU |
| STMN2_ | − | ATTG | 761 | TGAGATGGTGACTTC | 2767 | UGAGAUGGUGACUUC |
| intron1 | | | | CATTTATAGTTATAA | | CAUUUAUAGUUAUAA |
| STMN2_ | − | GTTA | 762 | AGATGGTGAAATTGT | 2768 | AGAUGGUGAAAUUGU |
| intron1 | | | | GAGATGGTGACTTCC | | GAGAUGGUGACUUCC |
| STMN2_ | − | ATTG | 763 | TTAAGATGGTGAAAT | 2769 | UUAAGAUGGUGAAAU |
| intron1 | | | | TGTGAGATGGTGACT | | UGUGAGAUGGUGACU |
| STMN2_ | − | TTTA | 764 | ACAAAATTGTTAAGA | 2770 | ACAAAAUUGUUAAGA |
| intron1 | | | | TGGTGAAATTGTGAG | | UGGUGAAAUUGUGAG |
| STMN2_ | − | GTTT | 765 | AACAAAATTGTTAAG | 2771 | AACAAAAUUGUUAAG |
| intron1 | | | | ATGGTGAAATTGTGA | | AUGGUGAAAUUGUGA |
| STMN2_ | − | ATTG | 766 | TAGGGCAGTTTAACA | 2772 | UAGGGCAGUUUAACA |
| intron1 | | | | AAATTGTTAAGATGG | | AAAUUGUUAAGAUGG |
| STMN2_ | − | CTTG | 767 | TAATATTGTAGGGCA | 2773 | UAAUAUUGUAGGGCA |
| intron1 | | | | GTTTAACAAAATTGT | | GUUUAACAAAAUUGU |
| STMN2_ | − | ATTA | 768 | TGTACTATCTTGTAA | 2774 | UGUACUAUCUUGUAA |
| intron1 | | | | TATTGTAGGGCAGTT | | UAUUGUAGGGCAGUU |
| STMN2_ | − | GTTA | 769 | CTAGTGTATCATTAT | 2775 | CUAGUGUAUCAUUAU |
| intron1 | | | | GTACTATCTTGTAAT | | GUACUAUCUUGUAAU |
| STMN2_ | − | TTTT | 770 | ATTTTTGTTCTCATA | 2776 | AUUUUUGUUCUCAUA |
| intron1 | | | | ATACCTGGCACAGGC | | AUACCUGGCACAGGC |
| STMN2_ | − | GTTG | 771 | ATGTTACTAGTGTAT | 2777 | AUGUUACUAGUGUAU |
| intron1 | | | | CATTATGTACTATCT | | CAUUAUGUACUAUCU |
| STMN2_ | − | TTTA | 772 | TTTTTGTTCTCATAA | 2778 | UUUUUGUUCUCAUAA |
| intron1 | | | | TACCTGGCACAGGCT | | UACCUGGCACAGGCU |
| STMN2_ | − | TTTT | 773 | TGTTCTCATAATACC | 2779 | UGUUCUCAUAAUACC |
| intron1 | | | | TGGCACAGGCTTCAG | | UGGCACAGGCUUCAG |
| STMN2_ | − | TTTT | 774 | GATAGGTAAATAATA | 2780 | GAUAGGUAAAUAAUA |
| intron1 | | | | TACACAACTTTATTA | | UACACAACUUUAUUA |
| STMN2_ | − | ATTT | 775 | TGATAGGTAAATAAT | 2781 | UGAUAGGUAAAUAAU |
| intron1 | | | | ATACACAACTTTATT | | AUACACAACUUUAUU |
| STMN2_ | − | ATTA | 776 | CATATAAATATTTTG | 2782 | CAUAUAAAUAUUUUG |
| intron1 | | | | ATAGGTAAATAATAT | | AUAGGUAAAUAAUAU |
| STMN2_ | − | TTTA | 777 | TATATTACATATAAA | 2783 | UAUAUUACAUAUAAA |
| intron1 | | | | TATTTTGATAGGTAA | | UAUUUUGAUAGGUAA |
| STMN2_ | − | ATTT | 778 | ATATATTACATATAA | 2784 | AUAUAUUACAUAUAA |
| intron1 | | | | ATATTTTGATAGGTA | | AUAUUUUGAUAGGUA |
| STMN2_ | − | TTTG | 779 | CATGAATGTGTATAT | 2785 | CAUGAAUGUGUAUAU |
| intron1 | | | | ATGTATGAAATAGGC | | AUGUAUGAAAUAGGC |
| STMN2_ | − | TTTT | 780 | GCATGAATGTGTATA | 2786 | GCAUGAAUGUGUAUA |
| intron1 | | | | TATGTATGAAATAGG | | UAUGUAUGAAAUAGG |
| STMN2_ | − | ATTT | 781 | TGCATGAATGTGTAT | 2787 | UGCAUGAAUGUGUAU |
| intron1 | | | | ATATGTATGAAATAG | | AUAUGUAUGAAAUAG |
| STMN2_ | − | CTTA | 782 | TTTTGCATGAATGTG | 2788 | UUUUGCAUGAAUGUG |
| intron1 | | | | TATATATGTATGAAA | | UAUAUAUGUAUGAAA |
| STMN2_ | − | ATTA | 783 | CAGGACAGTGGAGGG | 2789 | CAGGACAGUGGAGGG |
| intron1 | | | | AGTGCTAAACCTTAT | | AGUGCUAAACCUUAU |
| STMN2_ | − | TTTA | 784 | TTACAGGACAGTGGA | 2790 | UUACAGGACAGUGGA |
| intron1 | | | | GGGAGTGCTAAACCT | | GGGAGUGCUAAACCU |
| STMN2_ | − | TTTT | 785 | ATTACAGGACAGTGG | 2791 | AUUACAGGACAGUGG |
| intron1 | | | | AGGGAGTGCTAAACC | | AGGGAGUGCUAAACC |
| STMN2_ | − | GTTT | 786 | TATTACAGGACAGTG | 2792 | UAUUACAGGACAGUG |
| intron1 | | | | GAGGGAGTGCTAAAC | | GAGGGAGUGCUAAAC |
| STMN2_ | − | ATTC | 787 | TCACTGTGCATGTTT | 2793 | UCACUGUGCAUGUUU |
| intron1 | | | | TATTACAGGACAGTG | | UAUUACAGGACAGUG |
| STMN2_ | − | TTTA | 788 | AACTGAAGACAAATA | 2794 | AACUGAAGACAAAUA |
| intron1 | | | | TGCCTCGTGTATGAC | | UGCCUCGUGUAUGAC |
| STMN2_ | − | CTTT | 789 | AAACTGAAGACAAAT | 2795 | AAACUGAAGACAAAU |
| intron1 | | | | ATGCCTCGTGTATGA | | AUGCCUCGUGUAUGA |
| STMN2_ | − | GTTA | 790 | GTGACACTGACTATC | 2796 | GUGACACUGACUAUC |
| intron1 | | | | AATGACTTTAAACTG | | AAUGACUUUAAACUG |
| STMN2_ | − | TTTA | 791 | GTTAGTGACACTGAC | 2797 | GUUAGUGACACUGAC |
| intron1 | | | | TATCAATGACTTTAA | | UAUCAAUGACUUUAA |
| STMN2_ | − | CTTT | 792 | AGTTAGTGACACTGA | 2798 | AGUUAGUGACACUGA |
| intron1 | | | | CTATCAATGACTTTA | | CUAUCAAUGACUUUA |
| STMN2_ | − | TTTA | 793 | CTTTAGTTAGTGACA | 2799 | CUUUAGUUAGUGACA |
| intron1 | | | | CTGACTATCAATGAC | | CUGACUAUCAAUGAC |
| STMN2_ | − | TTTT | 794 | ACTTTAGTTAGTGAC | 2800 | ACUUUAGUUAGUGAC |
| intron1 | | | | ACTGACTATCAATGA | | ACUGACUAUCAAUGA |
| STMN2_ | − | ATTT | 795 | TACTTTAGTTAGTGA | 2801 | UACUUUAGUUAGUGA |
| intron1 | | | | CACTGACTATCAATG | | CACUGACUAUCAAUG |
| STMN2_ | − | GTTG | 796 | GTGCTCCAATCTATT | 2802 | GUGCUCCAAUCUAUU |
| intron1 | | | | TTACTTTAGTTAGTG | | UUACUUUAGUUAGUG |
| STMN2_ | − | CTTC | 797 | AGAACAAAGTTGGTG | 2803 | AGAACAAAGUUGGUG |
| intron1 | | | | CTCCAATCTATTTTA | | CUCCAAUCUAUUUUA |
| STMN2_ | − | GTTC | 798 | TCATAATACCTGGCA | 2804 | UCAUAAUACCUGGCA |
| intron1 | | | | CAGGCTTCAGAACAA | | CAGGCUUCAGAACAA |
| STMN2_ | − | TTTG | 799 | TTCTCATAATACCTG | 2805 | UUCUCAUAAUACCUG |
| intron1 | | | | GCACAGGCTTCAGAA | | GCACAGGCUUCAGAA |
| STMN2_ | − | TTTT | 800 | GTTCTCATAATACCT | 2806 | GUUCUCAUAAUACCU |
| intron1 | | | | GGCACAGGCTTCAGA | | GGCACAGGCUUCAGA |
| STMN2_ | − | ATTT | 801 | TTGTTCTCATAATAC | 2807 | UUGUUCUCAUAAUAC |
| intron1 | | | | CTGGCACAGGCTTCA | | CUGGCACAGGCUUCA |
| STMN2_ | − | CTTC | 802 | CTAGTTGATGTTACT | 2808 | CUAGUUGAUGUUACU |
| intron1 | | | | AGTGTATCATTATGT | | AGUGUAUCAUUAUGU |
| STMN2_ | − | CTTG | 803 | GTACTTCCTAGTTGA | 2809 | GUACUUCCUAGUUGA |
| intron1 | | | | TGTTACTAGTGTATC | | UGUUACUAGUGUAUC |
| STMN2_ | − | TTTG | 804 | GTGGATCTTGGTACT | 2810 | GUGGAUCUUGGUACU |
| intron1 | | | | TCCTAGTTGATGTTA | | UCCUAGUUGAUGUUA |
| STMN2_ | + | TTTT | 805 | ACTGAGAATCAGCAG | 2811 | ACUGAGAAUCAGCAG |
| intron1 | | | | CGTTTGAGGAGCTAG | | CGUUUGAGGAGCUAG |
| STMN2_ | + | ATTT | 806 | TACTGAGAATCAGCA | 2812 | UACUGAGAAUCAGCA |
| intron1 | | | | GCGTTTGAGGAGCTA | | GCGUUUGAGGAGCUA |
| STMN2_ | + | CTTC | 807 | CCAAATTTTACTGAG | 2813 | CCAAAUUUUACUGAG |
| intron1 | | | | AATCAGCAGCGTTTG | | AAUCAGCAGCGUUUG |
| STMN2_ | + | ATTA | 808 | AAATGCTTCCCAAAT | 2814 | AAAUGCUUCCCAAAU |
| intron1 | | | | TTTACTGAGAATCAG | | UUUACUGAGAAUCAG |
| STMN2_ | + | TTTA | 809 | ATTAAAATGCTTCCC | 2815 | AUUAAAAUGCUUCCC |
| intron1 | | | | AAATTTTACTGAGAA | | AAAUUUUACUGAGAA |
| STMN2_ | + | CTTT | 810 | AATTAAAATGCTTCC | 2816 | AAUUAAAAUGCUUCC |
| intron1 | | | | CAAATTTTACTGAGA | | CAAAUUUUACUGAGA |
| STMN2_ | + | ATTC | 811 | TTTAATTAAAATGCT | 2817 | UUUAAUUAAAAUGCU |
| intron1 | | | | TCCCAAATTTTACTG | | UCCCAAAUUUUACUG |
| STMN2_ | + | TTTA | 812 | ATGAGTCCATCAACC | 2818 | AUGAGUCCAUCAACC |
| intron1 | | | | AATCTGGCCAGAGAA | | AAUCUGGCCAGAGAA |
| STMN2_ | + | ATTT | 813 | AATGAGTCCATCAAC | 2819 | AAUGAGUCCAUCAAC |
| intron1 | | | | CAATCTGGCCAGAGA | | CAAUCUGGCCAGAGA |
| STMN2_ | + | TTTA | 814 | AATATTTAATGAGTC | 2820 | AAUAUUUAAUGAGUC |
| intron1 | | | | CATCAACCAATCTGG | | CAUCAACCAAUCUGG |
| STMN2_ | + | ATTT | 815 | AAATATTTAATGAGT | 2821 | AAAUAUUUAAUGAGU |
| intron1 | | | | CCATCAACCAATCTG | | CCAUCAACCAAUCUG |
| STMN2_ | | ATTA | 816 | CAAGATAGTACATAA | 2822 | CAAGAUAGUACAUAA |
| intron1 | | | | TGATACACTAGTAAC | | UGAUACACUAGUAAC |
| STMN2_ | + | GTTA | 817 | AACTGCCCTACAATA | 2823 | AACUGCCCUACAAUA |
| intron1 | | | | TTACAAGATAGTACA | | UUACAAGAUAGUACA |
| STMN2_ | + | TTTG | 818 | TTAAACTGCCCTACA | 2824 | UUAAACUGCCCUACA |
| intron1 | | | | ATATTACAAGATAGT | | AUAUUACAAGAUAGU |
| STMN2_ | + | TTTT | 819 | GTTAAACTGCCCTAC | 2825 | GUUAAACUGCCCUAC |
| intron1 | | | | AATATTACAAGATAG | | AAUAUUACAAGAUAG |
| STMN2_ | + | ATTT | 820 | TGTTAAACTGCCCTA | 2826 | UGUUAAACUGCCCUA |
| intron1 | | | | CAATATTACAAGATA | | CAAUAUUACAAGAUA |
| STMN2_ | + | CTTA | 821 | ACAATTTTGTTAAAC | 2827 | ACAAUUUUGUUAAAC |
| intron1 | | | | TGCCCTACAATATTA | | UGCCCUACAAUAUUA |
| STMN2_ | + | TTTC | 822 | ACCATCTTAACAATT | 2828 | ACCAUCUUAACAAUU |
| intron1 | | | | TTGTTAAACTGCCCT | | UUGUUAAACUGCCCU |
| STMN2_ | + | ATTT | 823 | CACCATCTTAACAAT | 2829 | CACCAUCUUAACAAU |
| intron1 | | | | TTTGTTAAACTGCCC | | UUUGUUAAACUGCCC |
| STMN2_ | + | TTTA | 824 | TAACTATAAATGGAA | 2830 | UAACUAUAAAUGGAA |
| intron1 | | | | GTCACCATCTCACAA | | GUCACCAUCUCACAA |
| STMN2_ | + | ATTT | 825 | ATAACTATAAATGGA | 2831 | AUAACUAUAAAUGGA |
| intron1 | | | | AGTCACCATCTCACA | | AGUCACCAUCUCACA |
| STMN2_ | + | GTTC | 826 | AATAGTAATAATGGT | 2832 | AAUAGUAAUAAUGGU |
| intron1 | | | | AATTTATAACTATAA | | AAUUUAUAACUAUAA |
| STMN2_ | + | TTTG | 827 | CAGAAAAAAAGATCC | 2833 | CAGAAAAAAAGAUCC |
| intron1 | | | | TGTACATGTCAGAAA | | UGUACAUGUCAGAAA |
| STMN2_ | + | TTTT | 828 | GCAGAAAAAAAGATC | 2834 | GCAGAAAAAAAGAUC |
| intron1 | | | | CTGTACATGTCAGAA | | CUGUACAUGUCAGAA |
| STMN2_ | + | CTTT | 829 | TGCAGAAAAAAAGAT | 2835 | UGCAGAAAAAAAGAU |
| intron1 | | | | CCTGTACATGTCAGA | | CCUGUACAUGUCAGA |
| STMN2_ | + | TTTA | 830 | GTCTTTTGCAGAAAA | 2836 | GUCUUUUGCAGAAAA |
| intron1 | | | | AAAGATCCTGTACAT | | AAAGAUCCUGUACAU |
| STMN2_ | + | ATTT | 831 | AGTCTTTTGCAGAAA | 2837 | AGUCUUUUGCAGAAA |
| intron1 | | | | AAAAGATCCTGTACA | | AAAAGAUCCUGUACA |
| STMN2_ | + | TTTA | 832 | CTGAGAATCAGCAGC | 2838 | CUGAGAAUCAGCAGC |
| intron1 | | | | GTTTGAGGAGCTAGC | | GUUUGAGGAGCUAGC |
| STMN2_ | + | GTTT | 833 | GAGGAGCTAGCCTCC | 2839 | GAGGAGCUAGCCUCC |
| intron1 | | | | ACCCCCAGAGGTTCT | | ACCCCCAGAGGUUCU |
| STMN2_ | + | TTTG | 834 | AGGAGCTAGCCTCCA | 2840 | AGGAGCUAGCCUCCA |
| intron1 | | | | CCCCCAGAGGTTCTC | | CCCCCAGAGGUUCUC |
| STMN2_ | + | GTTC | 835 | TCACTCTATTAGGTC | 2841 | UCACUCUAUUAGGUC |
| intron1 | | | | TGAAGCAGGTCCCAT | | UGAAGCAGGUCCCAU |
| STMN2_ | − | TTTT | 836 | GGTGGATCTTGGTAC | 2842 | GGUGGAUCUUGGUAC |
| intron1 | | | | TTCCTAGTTGATGTT | | UUCCUAGUUGAUGUU |
| STMN2_ | − | CTTT | 837 | TGGTGGATCTTGGTA | 2843 | UGGUGGAUCUUGGUA |
| intron1 | | | | CTTCCTAGTTGATGT | | CUUCCUAGUUGAUGU |
| STMN2_ | − | TTTC | 838 | AGCCTTTTGGTGGAT | 2844 | AGCCUUUUGGUGGAU |
| intron1 | | | | CTTGGTACTTCCTAG | | CUUGGUACUUCCUAG |
| STMN2_ | − | TTTT | 839 | CAGCCTTTTGGTGGA | 2845 | CAGCCUUUUGGUGGA |
| intron1 | | | | TCTTGGTACTTCCTA | | UCUUGGUACUUCCUA |
| STMN2_ | − | TTTT | 840 | TCAGCCTTTTGGTGG | 2846 | UCAGCCUUUUGGUGG |
| intron1 | | | | ATCTTGGTACTTCCT | | AUCUUGGUACUUCCU |
| STMN2_ | − | ATTT | 841 | TTCAGCCTTTTGGTG | 2847 | UUCAGCCUUUUGGUG |
| intron1 | | | | GATCTTGGTACTTCC | | GAUCUUGGUACUUCC |
| STMN2_ | − | TTTA | 842 | AATTTTTCAGCCTTT | 2848 | AAUUUUUCAGCCUUU |
| intron1 | | | | TGGTGGATCTTGGTA | | UGGUGGAUCUUGGUA |
| STMN2_ | − | ATTT | 843 | AAATTTTTCAGCCTT | 2849 | AAAUUUUUCAGCCUU |
| intron1 | | | | TTGGTGGATCTTGGT | | UUGGUGGAUCUUGGU |
| STMN2_ | − | ATTA | 844 | AATATTTAAATTTTT | 2850 | AAUAUUUAAAUUUUU |
| intron1 | | | | CAGCCTTTTGGTGGA | | CAGCCUUUUGGUGGA |
| STMN2_ | − | GTTG | 845 | ATGGACTCATTAAAT | 2851 | AUGGACUCAUUAAAU |
| intron1 | | | | ATTTAAATTTTTCAG | | AUUUAAAUUUUUCAG |
| STMN2_ | − | ATTG | 846 | GTTGATGGACTCATT | 2852 | GUUGAUGGACUCAUU |
| intron1 | | | | AAATATTTAAATTTT | | AAAUAUUUAAAUUUU |
| STMN2_ | − | ATTC | 847 | TCTGGCCAGATTGGT | 2853 | UCUGGCCAGAUUGGU |
| intron1 | | | | TGATGGACTCATTAA | | UGAUGGACUCAUUAA |
| STMN2_ | − | ATTA | 848 | AAGAATTCTCTGGCC | 2854 | AAGAAUUCUCUGGCC |
| intron1 | | | | AGATTGGTTGATGGA | | AGAUUGGUUGAUGGA |
| STMN2_ | − | TTTG | 849 | ATAGGTAAATAATAT | 2855 | AUAGGUAAAUAAUAU |
| intron1 | | | | ACACAACTTTATTAT | | ACACAACUUUAUUAU |
| STMN2_ | − | TTTA | 850 | ATTAAAGAATTCTCT | 2856 | AUUAAAGAAUUCUCU |
| intron1 | | | | GGCCAGATTGGTTGA | | GGCCAGAUUGGUUGA |
| STMN2_ | − | ATTT | 85 | TAATTAAAGAATTCT | 2857 | UAAUUAAAGAAUUCU |
| intron1 | | | | CTGGCCAGATTGGTT | | CUGGCCAGAUUGGUU |
| STMN2_ | − | TTTG | 852 | GGAAGCATTTTAATT | 2858 | GGAAGCAUUUUAAUU |
| intron1 | | | | AAAGAATTCTCTGGC | | AAAGAAUUCUCUGGC |
| STMN2_ | − | ATTT | 853 | GGGAAGCATTTTAAT | 2859 | GGGAAGCAUUUUAAU |
| intron1 | | | | TAAAGAATTCTCTGG | | UAAAGAAUUCUCUGG |
| STMN2_ | − | ATTC | 854 | TCAGTAAAATTTGGG | 2860 | UCAGUAAAAUUUGGG |
| intron1 | | | | AAGCATTTTAATTAA | | AAGCAUUUUAAUUAA |
| STMN2_ | − | CTTC | 855 | AGACCTAATAGAGTG | 2861 | AGACCUAAUAGAGUG |
| intron1 | | | | AGAACCTCTGGGGGT | | AGAACCUCUGGGGGU |
| STMN2_ | − | GTTA | 856 | GAAATGCAAATCCAT | 2862 | GAAAUGCAAAUCCAU |
| intron1 | | | | GGGACCTGCTTCAGA | | GGGACCUGCUUCAGA |
| STMN2_ | − | CTTG | 857 | TTAGAAATGCAAATC | 2863 | UUAGAAAUGCAAAUC |
| intron1 | | | | CATGGGACCTGCTTC | | CAUGGGACCUGCUUC |
| STMN2_ | − | GTTC | 858 | TGAATCAGCCTCATC | 2864 | UGAAUCAGCCUCAUC |
| intron1 | | | | AGCACCACCTGGGAG | | AGCACCACCUGGGAG |
| STMN2_ | + | TTTC | 859 | TAACAAGCTCCCAGG | 2865 | UAACAAGCUCCCAGG |
| intron1 | | | | TGGTGCTGATGAGGC | | UGGUGCUGAUGAGGC |
| STMN2_ | + | ATTT | 860 | CTAACAAGCTCCCAG | 2866 | CUAACAAGCUCCCAG |
| intron1 | | | | GTGGTGCTGATGAGG | | GUGGUGCUGAUGAGG |
| STMN2_ | + | TTTG | 86 | CATTTCTAACAAGCT | 2867 | CAUUUCUAACAAGCU |
| intron1 | | | | CCCAGGTGGTGCTGA | | CCCAGGUGGUGCUGA |
| STMN2_ | + | ATTT | 862 | GCATTTCTAACAAGC | 2868 | GCAUUUCUAACAAGC |
| intron1 | | | | TCCCAGGTGGTGCTG | | UCCCAGGUGGUGCUG |
| STMN2_ | + | ATTA | 863 | GGTCTGAAGCAGGTC | 2869 | GGUCUGAAGCAGGUC |
| intron1 | | | | CCATGGATTTGCATT | | CCAUGGAUUUGCAUU |
| STMN2_ | − | TTTT | 864 | AATTAAAGAATTCTC | 2870 | AAUUAAAGAAUUCUC |
| intron1 | | | | TGGCCAGATTGGTTG | | UGGCCAGAUUGGUUG |
| STMN2_ | − | CTTT | 865 | ATTATATGTAATATA | 2871 | AUUAUAUGUAAUAUA |
| intron1 | | | | TATATTATATGTTAT | | UAUAUUAUAUGUUAU |
| STMN2_ | − | TTTA | 866 | TTATATGTAATATAT | 2872 | UUAUAUGUAAUAUAU |
| intron1 | | | | ATATTATATGTTATA | | AUAUUAUAUGUUAUA |
| STMN2_ | − | ATTA | 867 | TATGTAATATATATA | 2873 | UAUGUAAUAUAUAUA |
| intron1 | | | | TTATATGTTATAATA | | UUAUAUGUUAUAAUA |
| STMN2_ | − | TTTG | 868 | TTAATGGAAGTTAAA | 2874 | UUAAUGGAAGUUAAA |
| intron1 | | | | CTTTATGGCTGCATT | | CUUUAUGGCUGCAUU |
| STMN2_ | − | CTTT | 869 | GTTAATGGAAGTTAA | 2875 | GUUAAUGGAAGUUAA |
| intron1 | | | | ACTTTATGGCTGCAT | | ACUUUAUGGCUGCAU |
| STMN2_ | − | TTTA | 870 | CTGTGAGCAGCTTTG | 2876 | CUGUGAGCAGCUUUG |
| intron1 | | | | TTAATGGAAGTTAAA | | UUAAUGGAAGUUAAA |
| STMN2_ | − | GTTT | 871 | ACTGTGAGCAGCTTT | 2877 | ACUGUGAGCAGCUUU |
| intron1 | | | | GTTAATGGAAGTTAA | | GUUAAUGGAAGUUAA |
| STMN2_ | − | ATTA | 872 | TAATAGGTTTACTGT | 2878 | UAAUAGGUUUACUGU |
| intron1 | | | | GAGCAGCTTTGTTAA | | GAGCAGCUUUGUUAA |
| STMN2_ | − | ATTA | 873 | TTATAATAGGTTTAC | 2879 | UUAUAAUAGGUUUAC |
| intron1 | | | | TGTGAGCAGCTTTGT | | UGUGAGCAGCUUUGU |
| STMN2_ | − | GTTG | 874 | CTCCTCACTAGGAAG | 2880 | CUCCUCACUAGGAAG |
| intron1 | | | | CCCAAACTGGGAAAC | | CCCAAACUGGGAAAC |
| STMN2_ | − | GTTA | 875 | GGTTGCTCCTCACTA | 2881 | GGUUGCUCCUCACUA |
| intron1 | | | | GGAAGCCCAAACTGG | | GGAAGCCCAAACUGG |
| STMN2_ | − | TTTC | 876 | GTGTGAGTTAGGTTG | 2882 | GUGUGAGUUAGGUUG |
| intron1 | | | | CTCCTCACTAGGAAG | | CUCCUCACUAGGAAG |
| STMN2_ | − | GTTT | 877 | CGTGTGAGTTAGGTT | 2883 | CGUGUGAGUUAGGUU |
| intron1 | | | | GCTCCTCACTAGGAA | | GCUCCUCACUAGGAA |
| STMN2_ | − | GTTG | 878 | TTTCGTGTGAGTTAG | 2884 | UUUCGUGUGAGUUAG |
| intron1 | | | | GTTGCTCCTCACTAG | | GUUGCUCCUCACUAG |
| STMN2_ | − | GTTG | 879 | GGGTTGTTTCGTGTG | 2885 | GGGUUGUUUCGUGUG |
| intron1 | | | | AGTTAGGTTGCTCCT | | AGUUAGGUUGCUCCU |
| STMN2_ | − | ATTA | 880 | TAAGTTGGGGTTGTT | 2886 | UAAGUUGGGGUUGUU |
| intron1 | | | | TCGTGTGAGTTAGGT | | UCGUGUGAGUUAGGU |
| STMN2_ | − | TTTG | 881 | TAACAGTCAATATAT | 2887 | UAACAGUCAAUAUAU |
| intron1 | | | | TATAAGTTGGGGTTG | | UAUAAGUUGGGGUUG |
| STMN2_ | − | TTTT | 882 | GTAACAGTCAATATA | 2888 | GUAACAGUCAAUAUA |
| intron1 | | | | TTATAAGTTGGGGTT | | UUAUAAGUUGGGGUU |
| STMN2_ | − | GTTT | 883 | TGTAACAGTCAATAT | 2889 | UGUAACAGUCAAUAU |
| intron1 | | | | ATTATAAGTTGGGGT | | AUUAUAAGUUGGGGU |
| STMN2_ | − | TTTC | 884 | TGGTCTCAGTTTTGT | 2890 | UGGUCUCAGUUUUGU |
| intron1 | | | | AACAGTCAATATATT | | AACAGUCAAUAUAUU |
| STMN2_ | − | TTTT | 885 | CTGGTCTCAGTTTTG | 2891 | CUGGUCUCAGUUUUG |
| intron1 | | | | TAACAGTCAATATAT | | UAACAGUCAAUAUAU |
| STMN2_ | − | ATTT | 886 | TCTGGTCTCAGTTTT | 2892 | UCUGGUCUCAGUUUU |
| intron1 | | | | GTAACAGTCAATATA | | GUAACAGUCAAUAUA |
| STMN2_ | − | CTTG | 887 | ATGGGATTTTCTGGT | 2893 | AUGGGAUUUUCUGGU |
| intron1 | | | | CTCAGTTTTGTAACA | | CUCAGUUUUGUAACA |
| STMN2_ | − | CTTC | 888 | CCGAGAGTCTGGAAA | 2894 | CCGAGAGUCUGGAAA |
| intron1 | | | | TGATAACAGTACCAT | | UGAUAACAGUACCAU |
| STMN2_ | − | GTTC | 889 | TTCCCGAGAGTCTGG | 2895 | UUCCCGAGAGUCUGG |
| intron1 | | | | AAATGATAACAGTAC | | AAAUGAUAACAGUAC |
| STMN2_ | − | ATTA | 890 | ATGTTCTTCCCGAGA | 2896 | AUGUUCUUCCCGAGA |
| intron1 | | | | GTCTGGAAATGATAA | | GUCUGGAAAUGAUAA |
| STMN2_ | − | GTTC | 891 | CCAGGGAGGCTGCAA | 2897 | CCAGGGAGGCUGCAA |
| intron1 | | | | TAAGTCTATCCTAAA | | UAAGUCUAUCCUAAA |
| STMN2_ | − | GTTC | 892 | TGAAGCAGAGTTCCC | 2898 | UGAAGCAGAGUUCCC |
| intron1 | | | | AGGGAGGCTGCAATA | | AGGGAGGCUGCAAUA |
| STMN2_ | − | ATTA | 893 | TGTTCTGAAGCAGAG | 2899 | UGUUCUGAAGCAGAG |
| intron1 | | | | TTCCCAGGGAGGCTG | | UUCCCAGGGAGGCUG |
| STMN2_ | − | ATTA | 894 | ATAAAAATAATTATG | 2900 | AUAAAAAUAAUUAUG |
| intron1 | | | | TTCTGAAGCAGAGTT | | UUCUGAAGCAGAGUU |
| STMN2_ | − | GTTA | 895 | ATGGAAGTTAAACTT | 2901 | AUGGAAGUUAAACUU |
| intron1 | | | | TATGGCTGCATTTCA | | UAUGGCUGCAUUUCA |
| STMN2_ | − | GTTA | 896 | AACTTTATGGCTGCA | 2902 | AACUUUAUGGCUGCA |
| intron1 | | | | TTTCATAAGGAAAAA | | UUUCAUAAGGAAAAA |
| STMN2_ | − | CTTT | 897 | ATGGCTGCATTTCAT | 2903 | AUGGCUGCAUUUCAU |
| intron1 | | | | AAGGAAAAAAAACTT | | AAGGAAAAAAAACUU |
| STMN2_ | − | TTTA | 898 | TGGCTGCATTTCATA | 2904 | UGGCUGCAUUUCAUA |
| intron1 | | | | AGGAAAAAAAACTTC | | AGGAAAAAAAACUUC |
| STMN2_ | − | ATTA | 899 | TTCCAGAAGAATAAC | 2905 | UUCCAGAAGAAUAAC |
| intron1 | | | | TGCTAAATGGGCACT | | UGCUAAAUGGGCACU |
| STMN2_ | − | GTTA | 900 | ATGTGCGAACTCCAA | 2906 | AUGUGCGAACUCCAA |
| intron1 | | | | CATCCAAAATACAAT | | CAUCCAAAAUACAAU |
| STMN2_ | − | CTTG | 901 | TACTAATGGTTAATG | 2907 | UACUAAUGGUUAAUG |
| intron1 | | | | TGCGAACTCCAACAT | | UGCGAACUCCAACAU |
| STMN2_ | − | ATTG | 902 | GGTACTTGTACTAAT | 2908 | GGUACUUGUACUAAU |
| intron1 | | | | GGTTAATGTGCGAAC | | GGUUAAUGUGCGAAC |
| STMN2_ | − | GTTA | 903 | TATTGGGTACTTGTA | 2909 | UAUUGGGUACUUGUA |
| intron1 | | | | CTAATGGTTAATGTG | | CUAAUGGUUAAUGUG |
| STMN2_ | − | ATTG | 904 | TTATATTGGGTACTT | 2910 | UUAUAUUGGGUACUU |
| intron1 | | | | GTACTAATGGTTAAT | | GUACUAAUGGUUAAU |
| STMN2_ | − | ATTA | 905 | TCCTGATGATCTATT | 2911 | UCCUGAUGAUCUAUU |
| intron1 | | | | GTTATATTGGGTACT | | GUUAUAUUGGGUACU |
| STMN2_ | − | TTTA | 906 | TTATCCTGATGATCT | 2912 | UUAUCCUGAUGAUCU |
| intron1 | | | | ATTGTTATATTGGGT | | AUUGUUAUAUUGGGU |
| STMN2_ | − | ATTT | 907 | ATTATCCTGATGATC | 2913 | AUUAUCCUGAUGAUC |
| intron1 | | | | TATTGTTATATTGGG | | UAUUGUUAUAUUGGG |
| STMN2_ | − | TTTA | 908 | TCCTGATATAAAGAC | 2914 | UCCUGAUAUAAAGAC |
| intron1 | | | | ATACAACTAAAAGAT | | AUACAACUAAAAGAU |
| STMN2_ | − | CTTT | 909 | ATCCTGATATAAAGA | 2915 | AUCCUGAUAUAAAGA |
| intron1 | | | | CATACAACTAAAAGA | | CAUACAACUAAAAGA |
| STMN2_ | − | ATTC | 910 | TCTTTATCCTGATAT | 2916 | UCUUUAUCCUGAUAU |
| intron1 | | | | AAAGACATACAACTA | | AAAGACAUACAACUA |
| STMN2_ | − | TTTC | 911 | ACTCAATTCTCTTTA | 2917 | ACUCAAUUCUCUUUA |
| intron1 | | | | TCCTGATATAAAGAC | | UCCUGAUAUAAAGAC |
| STMN2_ | − | GTTG | 912 | GAAATAAAAAGTAAC | 2918 | GAAAUAAAAAGUAAC |
| intron1 | | | | TCTGCATTAATAAAA | | UCUGCAUUAAUAAAA |
| STMN2_ | − | ATTT | 913 | CACTCAATTCTCTTT | 2919 | CACUCAAUUCUCUUU |
| intron1 | | | | ATCCTGATATAAAGA | | AUCCUGAUAUAAAGA |
| STMN2_ | − | GTTT | 914 | AGATAAATTTCACTC | 2920 | AGAUAAAUUUCACUC |
| intron1 | | | | AATTCTCTTTATCCT | | AAUUCUCUUUAUCCU |
| STMN2_ | − | TTTG | 915 | TGGGACTAGGTTTAG | 2921 | UGGGACUAGGUUUAG |
| intron1 | | | | ATAAATTTCACTCAA | | AUAAAUUUCACUCAA |
| STMN2_ | − | ATTT | 916 | GTGGGACTAGGTTTA | 2922 | GUGGGACUAGGUUUA |
| intron1 | | | | GATAAATTTCACTCA | | GAUAAAUUUCACUCA |
| STMN2_ | − | CTTG | 917 | TAAAAGTATTTGTGG | 2923 | UAAAAGUAUUUGUGG |
| intron1 | | | | GACTAGGTTTAGATA | | GACUAGGUUUAGAUA |
| STMN2_ | − | TTTA | 918 | ACATGCTCTCTTGTA | 2924 | ACAUGCUCUCUUGUA |
| intron1 | | | | AAAGTATTTGTGGGA | | AAAGUAUUUGUGGGA |
| STMN2_ | − | CTTT | 919 | AACATGCTCTCTTGT | 2925 | AACAUGCUCUCUUGU |
| intron1 | | | | AAAAGTATTTGTGGG | | AAAAGUAUUUGUGGG |
| STMN2_ | − | TTTA | 920 | CACTTTAACATGCTC | 2926 | CACUUUAACAUGCUC |
| intron1 | | | | TCTTGTAAAAGTATT | | UCUUGUAAAAGUAUU |
| STMN2_ | − | ATTT | 92 | ACACTTTAACATGCT | 2927 | ACACUUUAACAUGCU |
| intron1 | | | | CTCTTGTAAAAGTAT | | CUCUUGUAAAAGUAU |
| STMN2_ | − | TTTA | 922 | ATTTACACTTTAACA | 2928 | AUUUACACUUUAACA |
| intron1 | | | | TGCTCTCTTGTAAAA | | UGCUCUCUUGUAAAA |
| STMN2_ | − | ATTT | 923 | AATTTACACTTTAAC | 2929 | AAUUUACACUUUAAC |
| intron1 | | | | ATGCTCTCTTGTAAA | | AUGCUCUCUUGUAAA |
| STMN2_ | − | CTTC | 924 | CAAAGACAGAGTAGA | 2930 | CAAAGACAGAGUAGA |
| intron1 | | | | ATGCTAATAAAAATT | | AUGCUAAUAAAAAUU |
| STMN2_ | − | TTTC | 925 | ATAAGGAAAAAAAAC | 2931 | AUAAGGAAAAAAAAC |
| intron1 | | | | TTCCAAAGACAGAGT | | UUCCAAAGACAGAGU |
| STMN2_ | − | ATTT | 926 | CATAAGGAAAAAAAA | 2932 | CAUAAGGAAAAAAAA |
| intron1 | | | | CTTCCAAAGACAGAG | | CUUCCAAAGACAGAG |
| STMN2_ | − | TTTA | 927 | GATAAATTTCACTCA | 2933 | GAUAAAUUUCACUCA |
| intron1 | | | | ATTCTCTTTATCCTG | | AUUCUCUUUAUCCUG |
| STMN2_ | − | CTTG | 928 | CAGGCGTTGCTTTAC | 2934 | CAGGCGUUGCUUUAC |
| intron1 | | | | AATCTTTGTAAAAAA | | AAUCUUUGUAAAAAA |
| STMN2_ | − | TTTG | 929 | TTGGAAATAAAAAGT | 2935 | UUGGAAAUAAAAAGU |
| intron1 | | | | AACTCTGCATTAATA | | AACUCUGCAUUAAUA |
| STMN2_ | − | TTTT | 930 | TGTTGGAAATAAAAA | 2936 | UGUUGGAAAUAAAAA |
| intron1 | | | | GTAACTCTGCATTAA | | GUAACUCUGCAUUAA |
| STMN2_ | − | TTTT | 931 | GAACATTTTTTAGTC | 2937 | GAACAUUUUUUAGUC |
| intron1 | | | | TTCTATGCTTGCCTG | | UUCUAUGCUUGCCUG |
| STMN2_ | − | CTTT | 932 | TGAACATTTTTTAGT | 2938 | UGAACAUUUUUUAGU |
| intron1 | | | | CTTCTATGCTTGCCT | | CUUCUAUGCUUGCCU |
| STMN2_ | − | TTTC | 933 | TTTTGAACATTTTTT | 2939 | UUUUGAACAUUUUUU |
| intron1 | | | | AGTCTTCTATGCTTG | | AGUCUUCUAUGCUUG |
| STMN2_ | − | TTTT | 934 | CTTTTGAACATTTTT | 2940 | CUUUUGAACAUUUUU |
| intron1 | | | | TAGTCTTCTATGCTT | | UAGUCUUCUAUGCUU |
| STMN2_ | − | TTTT | 935 | TCTTTTGAACATTTT | 2941 | UCUUUUGAACAUUUU |
| intron1 | | | | TTAGTCTTCTATGCT | | UUAGUCUUCUAUGCU |
| STMN2_ | − | ATTT | 936 | TTCTTTTGAACATTT | 2942 | UUCUUUUGAACAUUU |
| intron1 | | | | TTTAGTCTTCTATGC | | UUUAGUCUUCUAUGC |
| STMN2_ | − | TTTA | 937 | ATTTTTCTTTTGAAC | 2943 | AUUUUUCUUUUGAAC |
| intron1 | | | | ATTTTTTAGTCTTCT | | AUUUUUUAGUCUUCU |
| STMN2_ | − | ATTT | 938 | AATTTTTCTTTTGAA | 2944 | AAUUUUUCUUUUGAA |
| intron1 | | | | CATTTTTTAGTCTTC | | CAUUUUUUAGUCUUC |
| STMN2_ | − | TTTC | 939 | TAAAAATGACAAGGT | 2945 | UAAAAAUGACAAGGU |
| intron1 | | | | CCCATATAGATAGAT | | CCCAUAUAGAUAGAU |
| STMN2_ | − | TTTT | 940 | CTAAAAATGACAAGG | 2946 | CUAAAAAUGACAAGG |
| intron1 | | | | TCCCATATAGATAGA | | UCCCAUAUAGAUAGA |
| STMN2_ | − | GTTT | 941 | TCTAAAAATGACAAG | 2947 | UCUAAAAAUGACAAG |
| intron1 | | | | GTCCCATATAGATAG | | GUCCCAUAUAGAUAG |
| STMN2_ | − | ATTC | 942 | AAAAGGATGAAGCAG | 2948 | AAAAGGAUGAAGCAG |
| intron1 | | | | GTGAATGTTTTCTAA | | GUGAAUGUUUUCUAA |
| STMN2_ | − | ATTA | 943 | TATGAAGATTCAAAA | 2949 | UAUGAAGAUUCAAAA |
| intron1 | | | | GGATGAAGCAGGTGA | | GGAUGAAGCAGGUGA |
| STMN2_ | − | CTTG | 944 | TATAGTATGCCCATC | 2950 | UAUAGUAUGCCCAUC |
| intron1 | | | | TCAGAGGGATTATAT | | UCAGAGGGAUUAUAU |
| STMN2_ | − | TTTA | 945 | AATAAGACAACTTGT | 2951 | AAUAAGACAACUUGU |
| intron1 | | | | ATAGTATGCCCATCT | | AUAGUAUGCCCAUCU |
| STMN2_ | − | CTTT | 946 | AAATAAGACAACTTG | 2952 | AAAUAAGACAACUUG |
| intron1 | | | | TATAGTATGCCCATC | | UAUAGUAUGCCCAUC |
| STMN2_ | − | TTTA | 947 | CCAATCTTTAAATAA | 2953 | CCAAUCUUUAAAUAA |
| intron1 | | | | GACAACTTGTATAGT | | GACAACUUGUAUAGU |
| STMN2_ | − | ATTT | 948 | ACCAATCTTTAAATA | 2954 | ACCAAUCUUUAAAUA |
| intron1 | | | | AGACAACTTGTATAG | | AGACAACUUGUAUAG |
| STMN2_ | − | CTTA | 949 | AATTTACCAATCTTT | 2955 | AAUUUACCAAUCUUU |
| intron1 | | | | AAATAAGACAACTTG | | AAAUAAGACAACUUG |
| STMN2_ | − | TTTG | 950 | AGCTTAAATTTACCA | 2956 | AGCUUAAAUUUACCA |
| intron1 | | | | ATCTTTAAATAAGAC | | AUCUUUAAAUAAGAC |
| STMN2_ | − | ATTT | 951 | GAGCTTAAATTTACC | 2957 | GAGCUUAAAUUUACC |
| intron1 | | | | AATCTTTAAATAAGA | | AAUCUUUAAAUAAGA |
| STMN2_ | − | ATTA | 952 | TTTGAGCTTAAATTT | 2958 | UUUGAGCUUAAAUUU |
| intron1 | | | | ACCAATCTTTAAATA | | ACCAAUCUUUAAAUA |
| STMN2_ | − | CTTG | 953 | CCACTGAATAAATTA | 2959 | CCACUGAAUAAAUUA |
| intron1 | | | | TTTGAGCTTAAATTT | | UUUGAGCUUAAAUUU |
| STMN2_ | − | GTTC | 954 | CGAGTCTGCCTCTGA | 2960 | CGAGUCUGCCUCUGA |
| intron1 | | | | GGCTTGCCACTGAAT | | GGCUUGCCACUGAAU |
| STMN2_ | − | ATTA | 955 | GACCTGTGTTCCGAG | 2961 | GACCUGUGUUCCGAG |
| intron1 | | | | TCTGCCTCTGAGGCT | | UCUGCCUCUGAGGCU |
| STMN2_ | − | GTTA | 956 | TAATATATATATAAT | 2962 | UAAUAUAUAUAUAAU |
| intron1 | | | | ATATATTAGACCTGT | | AUAUAUUAGACCUGU |
| STMN2_ | − | ATTA | 957 | TATGTTATAATATAT | 2963 | UAUGUUAUAAUAUAU |
| intron1 | | | | ATATAATATATATTA | | AUAUAAUAUAUAUUA |
| STMN2_ | − | TTTG | 958 | AACATTTTTTAGTCT | 2964 | AACAUUUUUUAGUCU |
| intron1 | | | | TCTATGCTTGCCTGC | | UCUAUGCUUGCCUGC |
| STMN2_ | − | ATTT | 959 | TTTAGTCTTCTATGC | 2965 | UUUAGUCUUCUAUGC |
| intron1 | | | | TTGCCTGCTCCTTTT | | UUGCCUGCUCCUUUU |
| STMN2_ | − | TTTT | 960 | TTAGTCTTCTATGCT | 2966 | UUAGUCUUCUAUGCU |
| intron1 | | | | TGCCTGCTCCTTTTA | | UGCCUGCUCCUUUUA |
| STMN2_ | − | TTTT | 961 | TAGTCTTCTATGCTT | 2967 | UAGUCUUCUAUGCUU |
| intron1 | | | | GCCTGCTCCTTTTAA | | GCCUGCUCCUUUUAA |
| STMN2_ | − | ATTT | 962 | TTGTTGGAAATAAAA | 2968 | UUGUUGGAAAUAAAA |
| intron1 | | | | AGTAACTCTGCATTA | | AGUAACUCUGCAUUA |
| STMN2_ | − | CTTA | 963 | AATAATAACAATAGA | 2969 | AAUAAUAACAAUAGA |
| intron1 | | | | TATTTTTGTTGGAAA | | UAUUUUUGUUGGAAA |
| STMN2_ | − | TTTC | 964 | TCAGATAAAGCTGTA | 2970 | UCAGAUAAAGCUGUA |
| intron1 | | | | AGACTTAAATAATAA | | AGACUUAAAUAAUAA |
| STMN2_ | − | ATTT | 965 | CTCAGATAAAGCTGT | 2971 | CUCAGAUAAAGCUGU |
| intron1 | | | | AAGACTTAAATAATA | | AAGACUUAAAUAAUA |
| STMN2_ | − | ATTG | 966 | GAATTTCTCAGATAA | 2972 | GAAUUUCUCAGAUAA |
| intron1 | | | | AGCTGTAAGACTTAA | | AGCUGUAAGACUUAA |
| STMN2_ | − | ATTA | 967 | TGAGAAGGGTGCTAA | 2973 | UGAGAAGGGUGCUAA |
| intron1 | | | | TTGGAATTTCTCAGA | | UUGGAAUUUCUCAGA |
| STMN2_ | − | TTTA | 968 | TTATGAGAAGGGTGC | 2974 | UUAUGAGAAGGGUGC |
| intron1 | | | | TAATTGGAATTTCTC | | UAAUUGGAAUUUCUC |
| STMN2_ | − | ATTT | 969 | ATTATGAGAAGGGTG | 2975 | AUUAUGAGAAGGGUG |
| intron1 | | | | CTAATTGGAATTTCT | | CUAAUUGGAAUUUCU |
| STMN2_ | − | TTTG | 970 | AATATTTATTATGAG | 2976 | AAUAUUUAUUAUGAG |
| intron1 | | | | AAGGGTGCTAATTGG | | AAGGGUGCUAAUUGG |
| STMN2_ | − | GTTT | 971 | GAATATTTATTATGA | 2977 | GAAUAUUUAUUAUGA |
| intron1 | | | | GAAGGGTGCTAATTG | | GAAGGGUGCUAAUUG |
| STMN2_ | − | TTTC | 972 | ATGTGTTTGAATATT | 2978 | AUGUGUUUGAAUAUU |
| intron1 | | | | TATTATGAGAAGGGT | | UAUUAUGAGAAGGGU |
| STMN2_ | − | TTTT | 973 | CATGTGTTTGAATAT | 2979 | CAUGUGUUUGAAUAU |
| intron1 | | | | TTATTATGAGAAGGG | | UUAUUAUGAGAAGGG |
| STMN2_ | − | TTTT | 974 | TCATGTGTTTGAATA | 2980 | UCAUGUGUUUGAAUA |
| intron1 | | | | TTTATTATGAGAAGG | | UUUAUUAUGAGAAGG |
| STMN2_ | − | TTTT | 975 | GTTGGAAATAAAAAG | 2981 | GUUGGAAAUAAAAAG |
| intron1 | | | | TAACTCTGCATTAAT | | UAACUCUGCAUUAAU |
| STMN2_ | − | ATTT | 976 | TTCATGTGTTTGAAT | 2982 | UUCAUGUGUUUGAAU |
| intron1 | | | | ATTTATTATGAGAAG | | AUUUAUUAUGAGAAG |
| STMN2_ | − | CTTT | 977 | GGTAATTTTTCATGT | 2983 | GGUAAUUUUUCAUGU |
| intron1 | | | | GTTTGAATATTTATT | | GUUUGAAUAUUUAUU |
| STMN2_ | − | ATTA | 978 | AAAGACTAGAACAAC | 2984 | AAAGACUAGAACAAC |
| intron1 | | | | TTTGGTAATTTTTCA | | UUUGGUAAUUUUUCA |
| STMN2_ | − | TTTA | 979 | AAGTGACAAGAGTGC | 2985 | AAGUGACAAGAGUGC |
| intron1 | | | | AGGATCATGTAATAT | | AGGAUCAUGUAAUAU |
| STMN2_ | − | TTTT | 980 | AAAGTGACAAGAGTG | 2986 | AAAGUGACAAGAGUG |
| intron1 | | | | CAGGATCATGTAATA | | CAGGAUCAUGUAAUA |
| STMN2_ | − | TTTT | 981 | TAAAGTGACAAGAGT | 2987 | UAAAGUGACAAGAGU |
| intron1 | | | | GCAGGATCATGTAAT | | GCAGGAUCAUGUAAU |
| STMN2_ | − | ATTT | 982 | TTAAAGTGACAAGAG | 2988 | UUAAAGUGACAAGAG |
| intron1 | | | | TGCAGGATCATGTAA | | UGCAGGAUCAUGUAA |
| STMN2_ | − | TTTA | 983 | AAAAACTATATAAGA | 2989 | AAAAACUAUAUAAGA |
| intron1 | | | | AAAAAATCATCAGAA | | AAAAAAUCAUCAGAA |
| STMN2_ | − | TTTT | 984 | AAAAAACTATATAAG | 2990 | AAAAAACUAUAUAAG |
| intron1 | | | | AAAAAAATCATCAGA | | AAAAAAAUCAUCAGA |
| STMN2_ | − | CTTT | 985 | TAAAAAACTATATAA | 2991 | UAAAAAACUAUAUAA |
| intron1 | | | | GAAAAAAATCATCAG | | GAAAAAAAUCAUCAG |
| STMN2_ | − | CTTG | 986 | CCTGCTCCTTTTAAA | 2992 | CCUGCUCCUUUUAAA |
| intron1 | | | | AAACTATATAAGAAA | | AAACUAUAUAAGAAA |
| STMN2_ | − | CTTC | 987 | TATGCTTGCCTGCTC | 2993 | UAUGCUUGCCUGCUC |
| intron1 | | | | CTTTTAAAAAACTAT | | CUUUUAAAAAACUAU |
| STMN2_ | − | TTTA | 988 | GTCTTCTATGCTTGC | 2994 | GUCUUCUAUGCUUGC |
| intron1 | | | | CTGCTCCTTTTAAAA | | CUGCUCCUUUUAAAA |
| STMN2_ | − | TTTT | 989 | AGTCTTCTATGCTTG | 2995 | AGUCUUCUAUGCUUG |
| intron1 | | | | CCTGCTCCTTTTAAA | | CCUGCUCCUUUUAAA |
| STMN2_ | − | TTTG | 990 | GTAATTTTTCATGTG | 2996 | GUAAUUUUUCAUGUG |
| intron1 | | | | TTTGAATATTTATTA | | UUUGAAUAUUUAUUA |
| STMN2_ | + | CTTG | 991 | GTGGATTTAGTCTTT | 2997 | GUGGAUUUAGUCUUU |
| intron1 | | | | TGCAGAAAAAAAGAT | | UGCAGAAAAAAAGAU |
| STMN2_ | − | GTTC | 992 | GGAAGTAAAATATTT | 2998 | GGAAGUAAAAUAUUU |
| intron1 | | | | TGTAAAGATTACCAT | | UGUAAAGAUUACCAU |
| STMN2_ | − | TTTT | 993 | GTAAAGATTACCATA | 2999 | GUAAAGAUUACCAUA |
| intron1 | | | | GATTTAAAAATGTTA | | GAUUUAAAAAUGUUA |
| STMN2_ | − | TTTA | 994 | CCTTTTTGTGGGGGA | 3000 | CCUUUUUGUGGGGGA |
| intron1 | | | | AAGGGATGAGGGCAA | | AAGGGAUGAGGGCAA |
| STMN2_ | − | ATTT | 995 | ACCTTTTTGTGGGGG | 3001 | ACCUUUUUGUGGGGG |
| intron1 | | | | AAAGGGATGAGGGCA | | AAAGGGAUGAGGGCA |
| STMN2_ | − | CTTA | 996 | AAATGAACAACTGGA | 3002 | AAAUGAACAACUGGA |
| intron1 | | | | GACAAATTTACCTTT | | GACAAAUUUACCUUU |
| STMN2_ | − | TTTA | 997 | TAACTTAAAATGAAC | 3003 | UAACUUAAAAUGAAC |
| intron1 | | | | AACTGGAGACAAATT | | AACUGGAGACAAAUU |
| STMN2_ | − | CTTT | 998 | ATAACTTAAAATGAA | 3004 | AUAACUUAAAAUGAA |
| intron1 | | | | CAACTGGAGACAAAT | | CAACUGGAGACAAAU |
| STMN2_ | − | TTTG | 999 | CTTTATAACTTAAAA | 3005 | CUUUAUAACUUAAAA |
| intron1 | | | | TGAACAACTGGAGAC | | UGAACAACUGGAGAC |
| STMN2_ | − | ATTT | 1000 | GCTTTATAACTTAAA | 3006 | GCUUUAUAACUUAAA |
| intron1 | | | | ATGAACAACTGGAGA | | AUGAACAACUGGAGA |
| STMN2_ | − | CTTA | 1001 | GCCACATGAACATAC | 3007 | GCCACAUGAACAUAC |
| intron1 | | | | ATAATCCTGGCAGGA | | AUAAUCCUGGCAGGA |
| STMN2_ | − | CTTG | 1002 | CACATGTATCTTAGC | 3008 | CACAUGUAUCUUAGC |
| intron1 | | | | CACATGAACATACAT | | CACAUGAACAUACAU |
| STMN2_ | − | CTTA | 1003 | GCAAGCACTTGCACA | 3009 | GCAAGCACUUGCACA |
| intron1 | | | | TGTATCTTAGCCACA | | UGUAUCUUAGCCACA |
| STMN2_ | − | GTTG | 1004 | GCACACAAACCCTGC | 3010 | GCACACAAACCCUGC |
| intron1 | | | | TCTTAGCAAGCACTT | | UCUUAGCAAGCACUU |
| STMN2_ | − | TTTC | 1005 | CAGCAATCGTTGGCA | 3011 | CAGCAAUCGUUGGCA |
| intron1 | | | | CACAAACCCTGCTCT | | CACAAACCCUGCUCU |
| STMN2_ | − | TTTT | 1006 | CCAGCAATCGTTGGC | 3012 | CCAGCAAUCGUUGGC |
| intron1 | | | | ACACAAACCCTGCTC | | ACACAAACCCUGCUC |
| STMN2_ | − | ATTT | 1007 | TCCAGCAATCGTTGG | 3013 | UCCAGCAAUCGUUGG |
| intron1 | | | | CACACAAACCCTGCT | | CACACAAACCCUGCU |
| STMN2_ | − | TTTG | 1008 | CAGAGAATTTTCCAG | 3014 | CAGAGAAUUUUCCAG |
| intron1 | | | | CAATCGTTGGCACAC | | CAAUCGUUGGCACAC |
| STMN2_ | − | CTTT | 1009 | GCAGAGAATTTTCCA | 3015 | GCAGAGAAUUUUCCA |
| intron1 | | | | GCAATCGTTGGCACA | | GCAAUCGUUGGCACA |
| STMN2_ | − | ATTC | 1010 | TTTGCAGAGAATTTT | 3016 | UUUGCAGAGAAUUUU |
| intron1 | | | | CCAGCAATCGTTGGC | | CCAGCAAUCGUUGGC |
| STMN2_ | − | ATTG | 1011 | CAGCCACAAACAATT | 3017 | CAGCCACAAACAAUU |
| intron1 | | | | CTTTGCAGAGAATTT | | CUUUGCAGAGAAUUU |
| STMN2_ | − | ATTC | 1012 | TCACCCATTGCAGCC | 3018 | UCACCCAUUGCAGCC |
| intron1 | | | | ACAAACAATTCTTTG | | ACAAACAAUUCUUUG |
| STMN2_ | − | ATTA | 1013 | TATATGTGTATTCTC | 3019 | UAUAUGUGUAUUCUC |
| intron1 | | | | ACCCATTGCAGCCAC | | ACCCAUUGCAGCCAC |
| STMN2_ | − | GTTG | 1014 | AAGATCATCTCAATT | 3020 | AAGAUCAUCUCAAUU |
| intron1 | | | | ATATATGTGTATTCT | | AUAUAUGUGUAUUCU |
| STMN2_ | − | CTTA | 1015 | TGTTGAAGATCATCT | 3021 | UGUUGAAGAUCAUCU |
| intron1 | | | | CAATTATATATGTGT | | CAAUUAUAUAUGUGU |
| STMN2_ | − | TTTA | 1016 | TAGATATAACCTTAT | 3022 | UAGAUAUAACCUUAU |
| intron1 | | | | GTTGAAGATCATCTC | | GUUGAAGAUCAUCUC |
| STMN2_ | − | ATTT | 1017 | ATAGATATAACCTTA | 3023 | AUAGAUAUAACCUUA |
| intron1 | | | | TGTTGAAGATCATCT | | UGUUGAAGAUCAUCU |
| STMN2_ | − | TTTA | 1018 | TATATTTATAGATAT | 3024 | UAUAUUUAUAGAUAU |
| intron1 | | | | AACCTTATGTTGAAG | | AACCUUAUGUUGAAG |
| STMN2_ | − | ATTT | 1019 | ATATATTTATAGATA | 3025 | AUAUAUUUAUAGAUA |
| intron1 | | | | TAACCTTATGTTGAA | | UAACCUUAUGUUGAA |
| STMN2_ | − | TTTG | 1020 | TGCATAAACTATATT | 3026 | UGCAUAAACUAUAUU |
| intron1 | | | | TATATATTTATAGAT | | UAUAUAUUUAUAGAU |
| STMN2_ | − | CTTT | 1021 | TTGTGGGGGAAAGGG | 3027 | UUGUGGGGGAAAGGG |
| intron1 | | | | ATGAGGGCAATTAGG | | AUGAGGGCAAUUAGG |
| STMN2_ | − | TTTT | 1022 | GTGCATAAACTATAT | 3028 | GUGCAUAAACUAUAU |
| intron1 | | | | TTATATATTTATAGA | | UUAUAUAUUUAUAGA |
| STMN2_ | − | TTTT | 1023 | TGTGGGGGAAAGGGA | 3029 | UGUGGGGGAAAGGGA |
| intron1 | | | | TGAGGGCAATTAGGA | | UGAGGGCAAUUAGGA |
| STMN2_ | − | TTTG | 1024 | TGGGGGAAAGGGATG | 303 | UGGGGGAAAGGGAUG |
| intron1 | | | | AGGGCAATTAGGAGG | 0 | AGGGCAAUUAGGAGG |
| STMN2_ | − | GTTG | 1025 | TGGACTGCGGGGCTG | 3031 | UGGACUGCGGGGCUG |
| intron1 | | | | AAAAAAGAGGTTCCA | | AAAAAAGAGGUUCCA |
| STMN2_ | − | CTTG | 1026 | GCTGGGAGGGGCTCG | 3032 | GCUGGGAGGGGCUCG |
| intron1 | | | | GTGCTGGGGCTGAGA | | GUGCUGGGGCUGAGA |
| STMN2_ | − | TTTC | 1027 | TGCAGAGCCACCCGC | 3033 | UGCAGAGCCACCCGC |
| intron1 | | | | TTGGCTGGGAGGGGC | | UUGGCUGGGAGGGGC |
| STMN2_ | − | TTTT | 1028 | CTGCAGAGCCACCCG | 3034 | CUGCAGAGCCACCCG |
| intron1 | | | | CTTGGCTGGGAGGGG | | CUUGGCUGGGAGGGG |
| STMN2_ | − | CTTT | 1029 | TCTGCAGAGCCACCC | 3035 | UCUGCAGAGCCACCC |
| intron1 | | | | GCTTGGCTGGGAGGG | | GCUUGGCUGGGAGGG |
| STMN2_ | − | TTTG | 1030 | TGTGGCCGGGGGGGG | 3036 | UGUGGCCGGGGGGGG |
| intron1 | | | | CTCGAGCCAGCTTTT | | CUCGAGCCAGCUUUU |
| STMN2_ | − | CTTT | 1031 | GTGTGGCCGGGGGGG | 3037 | GUGUGGCCGGGGGGG |
| intron1 | | | | GCTCGAGCCAGCTTT | | GCUCGAGCCAGCUUU |
| STMN2_ | − | CTTG | 1032 | GGCTGGGGGAAAAAA | 3038 | GGCUGGGGGAAAAAA |
| intron1 | | | | AGCCCCGAGCTCCGC | | AGCCCCGAGCUCCGC |
| STMN2_ | − | ATTC | 1033 | TGGAAAATCATAGAG | 3039 | UGGAAAAUCAUAGAG |
| intron1 | | | | AACAGAGGGTGGGCG | | AACAGAGGGUGGGCG |
| STMN2_ | − | CTTA | 1034 | GAGAAGCCCCTCGCG | 3040 | GAGAAGCCCCUCGCG |
| intron1 | | | | GGGTCTCCATTCTGG | | GGGUCUCCAUUCUGG |
| STMN2_ | − | ATTG | 1035 | TGGAAAGCGGGGGTA | 3041 | UGGAAAGCGGGGGUA |
| intron1 | | | | GCTCAGGACACTGCG | | GCUCAGGACACUGCG |
| STMN2_ | − | TTTC | 1036 | TGGACGTGCGAGTGA | 3042 | UGGACGUGCGAGUGA |
| intron1 | | | | ACTGCGAATTGTGGA | | ACUGCGAAUUGUGGA |
| STMN2_ | − | CTTT | 1037 | CTGGACGTGCGAGTG | 3043 | CUGGACGUGCGAGUG |
| intron1 | | | | AACTGCGAATTGTGG | | AACUGCGAAUUGUGG |
| STMN2_ | − | ATTC | 1038 | TCAGAACCTTTCTGG | 3044 | UCAGAACCUUUCUGG |
| intron1 | | | | ACGTGCGAGTGAACT | | ACGUGCGAGUGAACU |
| STMN2_ | − | TTTC | 1039 | TGAGGGGTGCAGAAA | 3045 | UGAGGGGUGCAGAAA |
| intron1 | | | | GCGAGGCGAGATCGC | | GCGAGGCGAGAUCGC |
| STMN2_ | − | CTTT | 1040 | CTGAGGGGTGCAGAA | 3046 | CUGAGGGGUGCAGAA |
| intron1 | | | | AGCGAGGCGAGATCG | | AGCGAGGCGAGAUCG |
| STMN2_ | − | TTTG | 1041 | CAGCCACTAGCCTGC | 3047 | CAGCCACUAGCCUGC |
| intron1 | | | | AGCGGAAACCTTTCT | | AGCGGAAACCUUUCU |
| STMN2_ | − | GTTT | 1042 | GCAGCCACTAGCCTG | 3048 | GCAGCCACUAGCCUG |
| intron1 | | | | CAGCGGAAACCTTTC | | CAGCGGAAACCUUUC |
| STMN2_ | − | TTTG | 1043 | AAATGATAATAATAC | 3049 | AAAUGAUAAUAAUAC |
| intron1 | | | | TGATGATGACGATGA | | UGAUGAUGACGAUGA |
| STMN2_ | − | ATTT | 1044 | GAAATGATAATAATA | 3050 | GAAAUGAUAAUAAUA |
| intron1 | | | | CTGATGATGACGATG | | CUGAUGAUGACGAUG |
| STMN2_ | − | ATTA | 1045 | AATAATAACAACGAT | 3051 | AAUAAUAACAACGAU |
| intron1 | | | | TTGAAATGATAATAA | | UUGAAAUGAUAAUAA |
| STMN2_ | − | TTTG | 1046 | AACAAATGAGAACAA | 3052 | AACAAAUGAGAACAA |
| intron1 | | | | ACAAGGCTACTGAAT | | ACAAGGCUACUGAAU |
| STMN2_ | − | TTTT | 1047 | GAACAAATGAGAACA | 3053 | GAACAAAUGAGAACA |
| intron1 | | | | AACAAGGCTACTGAA | | AACAAGGCUACUGAA |
| STMN2_ | − | CTTT | 1048 | TGAACAAATGAGAAC | 3054 | UGAACAAAUGAGAAC |
| intron1 | | | | AAACAAGGCTACTGA | | AAACAAGGCUACUGA |
| STMN2_ | − | CTTA | 1049 | ACCAAGAGCAATCCA | 3055 | ACCAAGAGCAAUCCA |
| intron1 | | | | CGTCCCTTTTGAACA | | CGUCCCUUUUGAACA |
| STMN2_ | − | GTTA | 1050 | ATCCTTAACCAAGAG | 3056 | AUCCUUAACCAAGAG |
| intron1 | | | | CAATCCACGTCCCTT | | CAAUCCACGUCCCUU |
| STMN2_ | − | ATTA | 1051 | GGAGGAAGCAAAGCG | 3057 | GGAGGAAGCAAAGCG |
| intron1 | | | | AACGCAACAAGGGTT | | AACGCAACAAGGGUU |
| STMN2_ | − | TTTT | 1052 | GTGGGGGAAAGGGAT | 3058 | GUGGGGGAAAGGGAU |
| intron1 | | | | GAGGGCAATTAGGAG | | GAGGGCAAUUAGGAG |
| STMN2_ | − | ATTT | 1053 | TGTGCATAAACTATA | 3059 | UGUGCAUAAACUAUA |
| intron1 | | | | TTTATATATTTATAG | | UUUAUAUAUUUAUAG |
| STMN2_ | − | CTTA | 1054 | AAATTTTGTGCATAA | 3060 | AAAUUUUGUGCAUAA |
| intron1 | | | | ACTATATTTATATAT | | ACUAUAUUUAUAUAU |
| STMN2_ | − | TTTC | 1055 | AGGGGAAAAAACTTA | 3061 | AGGGGAAAAAACUUA |
| intron1 | | | | AAATTTTGTGCATAA | | AAAUUUUGUGCAUAA |
| STMN2_ | − | ATTA | 1056 | ATTTCAAAATCTATT | 3062 | AUUUCAAAAUCUAUU |
| intron1 | | | | ATTTTAATACTGCAG | | AUUUUAAUACUGCAG |
| STMN2_ | − | ATTG | 1057 | GAATTAATTTCAAAA | 3063 | GAAUUAAUUUCAAAA |
| intron1 | | | | TCTATTATTTTAATA | | UCUAUUAUUUUAAUA |
| STMN2_ | − | TTTG | 1058 | AAATTGGAATTAATT | 3064 | AAAUUGGAAUUAAUU |
| intron1 | | | | TCAAAATCTATTATT | | UCAAAAUCUAUUAUU |
| STMN2_ | − | CTTT | 1059 | GAAATTGGAATTAAT | 3065 | GAAAUUGGAAUUAAU |
| intron1 | | | | TTCAAAATCTATTAT | | UUCAAAAUCUAUUAU |
| STMN2_ | − | ATTA | 1060 | TCTTTGAAATTGGAA | 3066 | UCUUUGAAAUUGGAA |
| intron1 | | | | TTAATTTCAAAATCT | | UUAAUUUCAAAAUCU |
| STMN2_ | − | ATTA | 1061 | ATTATCTTTGAAATT | 3067 | AUUAUCUUUGAAAUU |
| intron1 | | | | GGAATTAATTTCAAA | | GGAAUUAAUUUCAAA |
| STMN2_ | − | TTTA | 1062 | ATGAATCAGGAAAAA | 3068 | AUGAAUCAGGAAAAA |
| intron1 | | | | AGCACTCGCCCTGAT | | AGCACUCGCCCUGAU |
| STMN2_ | − | GTTT | 1063 | AATGAATCAGGAAAA | 3069 | AAUGAAUCAGGAAAA |
| intron1 | | | | AAGCACTCGCCCTGA | | AAGCACUCGCCCUGA |
| STMN2_ | − | ATTG | 1064 | TTTAATGAATCAGGA | 3070 | UUUAAUGAAUCAGGA |
| intron1 | | | | AAAAAGCACTCGCCC | | AAAAAGCACUCGCCC |
| STMN2_ | − | CTTA | 1065 | CAATCATGCTGAATA | 3071 | CAAUCAUGCUGAAUA |
| intron1 | | | | CATAATTGTTTAATG | | CAUAAUUGUUUAAUG |
| STMN2_ | − | ATTA | 1066 | TATGCACCTCTTACA | 3072 | UAUGCACCUCUUACA |
| intron1 | | | | ATCATGCTGAATACA | | AUCAUGCUGAAUACA |
| STMN2_ | − | ATTA | 1067 | GAAAAGATAATGGGG | 3073 | GAAAAGAUAAUGGGG |
| intron1 | | | | AATATTATATGCACC | | AAUAUUAUAUGCACC |
| STMN2_ | − | CTTC | 1068 | ATTAGAAAAGATAAT | 3074 | AUUAGAAAAGAUAAU |
| intron1 | | | | GGGGAATATTATATG | | GGGGAAUAUUAUAUG |
| STMN2_ | − | ATTC | 1069 | AGAAGGTGCCCACTT | 3075 | AGAAGGUGCCCACUU |
| intron1 | | | | CATTAGAAAAGATAA | | CAUUAGAAAAGAUAA |
| STMN2_ | − | CTTA | 1070 | TATATCCATTCAGAA | 3076 | UAUAUCCAUUCAGAA |
| intron1 | | | | GGTGCCCACTTCATT | | GGUGCCCACUUCAUU |
| STMN2_ | − | GTTA | 1071 | CTTATATATCCATTC | 307 | CUUAUAUAUCCAUUC |
| intron1 | | | | AGAAGGTGCCCACTT | 7 | AGAAGGUGCCCACUU |
| STMN2_ | − | TTTC | 1072 | TAGTTACTTATATAT | 3078 | UAGUUACUUAUAUAU |
| intron1 | | | | CCATTCAGAAGGTGC | | CCAUUCAGAAGGUGC |
| STMN2_ | − | ATTT | 1073 | CTAGTTACTTATATA | 3079 | CUAGUUACUUAUAUA |
| intron1 | | | | TCCATTCAGAAGGTG | | UCCAUUCAGAAGGUG |
| STMN2_ | − | TTTC | 1074 | ATTTCTAGTTACTTA | 3080 | AUUUCUAGUUACUUA |
| intron1 | | | | TATATCCATTCAGAA | | UAUAUCCAUUCAGAA |
| STMN2_ | − | TTTT | 1075 | CATTTCTAGTTACTT | 3081 | CAUUUCUAGUUACUU |
| intron1 | | | | ATATATCCATTCAGA | | AUAUAUCCAUUCAGA |
| STMN2_ | − | CTTT | 1076 | TCATTTCTAGTTACT | 3082 | UCAUUUCUAGUUACU |
| intron1 | | | | TATATATCCATTCAG | | UAUAUAUCCAUUCAG |
| STMN2_ | − | ATTC | 1077 | TGACCAAATCCTCAG | 3083 | UGACCAAAUCCUCAG |
| intron1 | | | | CTTTTCATTTCTAGT | | CUUUUCAUUUCUAGU |
| STMN2_ | − | TTTA | 1078 | TCCTGAAATTCTGAC | 3084 | UCCUGAAAUUCUGAC |
| intron1 | | | | CAAATCCTCAGCTTT | | CAAAUCCUCAGCUUU |
| STMN2_ | − | TTTT | 1079 | ATCCTGAAATTCTGA | 3085 | AUCCUGAAAUUCUGA |
| intron1 | | | | CCAAATCCTCAGCTT | | CCAAAUCCUCAGCUU |
| STMN2_ | − | GTTT | 1080 | TATCCTGAAATTCTG | 3086 | UAUCCUGAAAUUCUG |
| intron1 | | | | ACCAAATCCTCAGCT | | ACCAAAUCCUCAGCU |
| STMN2_ | − | TTTC | 1081 | AGTTTTATCCTGAAA | 3087 | AGUUUUAUCCUGAAA |
| intron1 | | | | TTCTGACCAAATCCT | | UUCUGACCAAAUCCU |
| STMN2_ | − | CTTT | 1082 | CAGTTTTATCCTGAA | 3088 | CAGUUUUAUCCUGAA |
| intron1 | | | | ATTCTGACCAAATCC | | AUUCUGACCAAAUCC |
| STMN2_ | − | ATTT | 1083 | CAAAATCTATTATTT | 3089 | CAAAAUCUAUUAUUU |
| intron1 | | | | TAATACTGCAGAAGT | | UAAUACUGCAGAAGU |
| STMN2_ | − | TTTC | 1084 | AAAATCTATTATTTT | 3090 | AAAAUCUAUUAUUUU |
| intron1 | | | | AATACTGCAGAAGTA | | AAUACUGCAGAAGUA |
| STMN2_ | − | ATTA | 1085 | TTTTAATACTGCAGA | 3091 | UUUUAAUACUGCAGA |
| intron1 | | | | AGTAGTGTTTTTTTC | | AGUAGUGUUUUUUUC |
| STMN2_ | − | ATTT | 1086 | TAATACTGCAGAAGT | 3092 | UAAUACUGCAGAAGU |
| intron1 | | | | AGTGTTTTTTTCATG | | AGUGUUUUUUUCAUG |
| STMN2_ | − | GTTT | 1087 | CAGGGGAAAAAACTT | 3093 | CAGGGGAAAAAACUU |
| intron1 | | | | AAAATTTTGTGCATA | | AAAAUUUUGUGCAUA |
| STMN2_ | − | GTTG | 1088 | GAAGAACAGTTTCAG | 3094 | GAAGAACAGUUUCAG |
| intron1 | | | | GGGAAAAAACTTAAA | | GGGAAAAAACUUAAA |
| STMN2_ | − | ATTG | 1089 | AGGCTGTATCAAGAA | 3095 | AGGCUGUAUCAAGAA |
| intron1 | | | | TCAGCAGTTGGAAGA | | UCAGCAGUUGGAAGA |
| STMN2_ | − | TTTC | 1090 | ACGATCCATGTATCT | 3096 | ACGAUCCAUGUAUCU |
| intron1 | | | | GTGTAGGATTGAGGC | | GUGUAGGAUUGAGGC |
| STMN2_ | − | ATTT | 1091 | CACGATCCATGTATC | 3097 | CACGAUCCAUGUAUC |
| intron1 | | | | TGTGTAGGATTGAGG | | UGUGUAGGAUUGAGG |
| STMN2_ | − | TTTG | 1092 | GATGGCGGCTACCAT | 3098 | GAUGGCGGCUACCAU |
| intron1 | | | | TTCACGATCCATGTA | | UUCACGAUCCAUGUA |
| STMN2_ | − | ATTT | 1093 | GGATGGCGGCTACCA | 3099 | GGAUGGCGGCUACCA |
| intron1 | | | | TTTCACGATCCATGT | | UUUCACGAUCCAUGU |
| STMN2_ | − | TTTA | 1094 | TTTGGATGGCGGCTA | 3100 | UUUGGAUGGCGGCUA |
| intron1 | | | | CCATTTCACGATCCA | | CCAUUUCACGAUCCA |
| STMN2_ | − | TTTT | 1095 | ATTTGGATGGCGGCT | 3101 | AUUUGGAUGGCGGCU |
| intron1 | | | | ACCATTTCACGATCC | | ACCAUUUCACGAUCC |
| STMN2_ | − | TTTT | 1096 | TATTTGGATGGCGGC | 3102 | UAUUUGGAUGGCGGC |
| intron1 | | | | TACCATTTCACGATC | | UACCAUUUCACGAUC |
| STMN2_ | − | ATTT | 1097 | TTATTTGGATGGCGG | 3103 | UUAUUUGGAUGGCGG |
| intron1 | | | | CTACCATTTCACGAT | | CUACCAUUUCACGAU |
| STMN2_ | − | TTTG | 1098 | GGGTGGGATTTTTAT | 3104 | GGGUGGGAUUUUUAU |
| intron1 | | | | TTGGATGGCGGCTAC | | UUGGAUGGCGGCUAC |
| STMN2_ | − | ATTT | 1099 | GGGGTGGGATTTTTA | 3105 | GGGGUGGGAUUUUUA |
| intron1 | | | | TTTGGATGGCGGCTA | | UUUGGAUGGCGGCUA |
| STMN2_ | − | GTTC | 1100 | CAGGACTGCATACAG | 3106 | CAGGACUGCAUACAG |
| intron1 | | | | CTCAACTGCCCCTCC | | CUCAACUGCCCCUCC |
| STMN2_ | − | TTTG | 1101 | TCATATTTGGGGTGG | 3107 | UCAUAUUUGGGGUGG |
| intron1 | | | | GATTTTTATTTGGAT | | GAUUUUUAUUUGGAU |
| STMN2_ | − | CTTG | 1102 | CGTTTGTCATATTTG | 3108 | CGUUUGUCAUAUUUG |
| intron1 | | | | GGGTGGGATTTTTAT | | GGGUGGGAUUUUUAU |
| STMN2_ | − | ATTA | 1103 | TGGCCAGAAAGGATG | 3109 | UGGCCAGAAAGGAUG |
| intron1 | | | | CTTGCGTTTGTCATA | | CUUGCGUUUGUCAUA |
| STMN2_ | − | GTTA | 1104 | AATTATGGCCAGAAA | 3110 | AAUUAUGGCCAGAAA |
| intron1 | | | | GGATGCTTGCGTTTG | | GGAUGCUUGCGUUUG |
| STMN2_ | − | TTTG | 1105 | CAAATGCAGTTAAAT | 3111 | CAAAUGCAGUUAAAU |
| intron1 | | | | TATGGCCAGAAAGGA | | UAUGGCCAGAAAGGA |
| STMN2_ | − | ATTT | 1106 | GCAAATGCAGTTAAA | 3112 | GCAAAUGCAGUUAAA |
| intron1 | | | | TTATGGCCAGAAAGG | | UUAUGGCCAGAAAGG |
| STMN2_ | − | TTTC | 1107 | ATGATTTGCAAATGC | 3113 | AUGAUUUGCAAAUGC |
| intron1 | | | | AGTTAAATTATGGCC | | AGUUAAAUUAUGGCC |
| STMN2_ | − | TTTT | 1108 | CATGATTTGCAAATG | 3114 | CAUGAUUUGCAAAUG |
| intron1 | | | | CAGTTAAATTATGGC | | CAGUUAAAUUAUGGC |
| STMN2_ | − | TTTT | 1109 | TCATGATTTGCAAAT | 3115 | UCAUGAUUUGCAAAU |
| intron1 | | | | GCAGTTAAATTATGG | | GCAGUUAAAUUAUGG |
| STMN2_ | − | TTTT | 1110 | TTCATGATTTGCAAA | 3116 | UUCAUGAUUUGCAAA |
| intron1 | | | | TGCAGTTAAATTATG | | UGCAGUUAAAUUAUG |
| STMN2_ | − | TTTT | 1111 | TTTCATGATTTGCAA | 3117 | UUUCAUGAUUUGCAA |
| intron1 | | | | ATGCAGTTAAATTAT | | AUGCAGUUAAAUUAU |
| STMN2_ | − | GTTT | 1112 | TTTTCATGATTTGCA | 3118 | UUUUCAUGAUUUGCA |
| intron1 | | | | AATGCAGTTAAATTA | | AAUGCAGUUAAAUUA |
| STMN2_ | − | TTTA | 1113 | ATACTGCAGAAGTAG | 3119 | AUACUGCAGAAGUAG |
| intron1 | | | | TGTTTTTTTCATGAT | | UGUUUUUUUCAUGAU |
| STMN2_ | − | TTTT | 1114 | AATACTGCAGAAGTA | 3120 | AAUACUGCAGAAGUA |
| intron1 | | | | GTGTTTTTTTCATGA | | GUGUUUUUUUCAUGA |
| STMN2_ | − | GTTT | 1115 | GTCATATTTGGGGTG | 3121 | GUCAUAUUUGGGGUG |
| intron1 | | | | GGATTTTTATTTGGA | | GGAUUUUUAUUUGGA |
| STMN2_ | − | ATTC | 1116 | CTCTTCCCCGCCAGT | 3122 | CUCUUCCCCGCCAGU |
| intron1 | | | | CTCGGAGCCTGAGGT | | CUCGGAGCCUGAGGU |
| STMN2_ | − | CTTC | 1117 | CCCGCCAGTCTCGGA | 3123 | CCCGCCAGUCUCGGA |
| intron1 | | | | GCCTGAGGTCTCCCC | | GCCUGAGGUCUCCCC |
| STMN2_ | − | CTTT | 1118 | CGGCAGCTTTCCCTG | 3124 | CGGCAGCUUUCCCUG |
| intron1 | | | | TCTCCGCATCCTGCA | | UCUCCGCAUCCUGCA |
| STMN2_ | − | TTTC | 1119 | CAAAATGTCCCTTAA | 3125 | CAAAAUGUCCCUUAA |
| intron1 | | | | GCCCATTTAAGGCAA | | GCCCAUUUAAGGCAA |
| STMN2_ | − | CTTT | 1120 | CCAAAATGTCCCTTA | 3126 | CCAAAAUGUCCCUUA |
| intron1 | | | | AGCCCATTTAAGGCA | | AGCCCAUUUAAGGCA |
| STMN2_ | − | GTTA | 1121 | TAAAGCACTTTCCAA | 3127 | UAAAGCACUUUCCAA |
| intron1 | | | | AATGTCCCTTAAGCC | | AAUGUCCCUUAAGCC |
| STMN2_ | − | CTTA | 1122 | AACTAGAGAAGAAAT | 3128 | AACUAGAGAAGAAAU |
| intron1 | | | | AAAAAAAAAAAAGGT | | AAAAAAAAAAAAGGU |
| STMN2_ | − | CTTC | 1123 | TTAAACTAGAGAAGA | 3129 | UUAAACUAGAGAAGA |
| intron1 | | | | AATAAAAAAAAAAAA | | AAUAAAAAAAAAAAA |
| STMN2_ | − | TTTC | 1124 | TTCTTAAACTAGAGA | 3130 | UUCUUAAACUAGAGA |
| intron1 | | | | AGAAATAAAAAAAAA | | AGAAAUAAAAAAAAA |
| STMN2_ | − | TTTT | 1125 | CTTCTTAAACTAGAG | 3131 | CUUCUUAAACUAGAG |
| intron1 | | | | AAGAAATAAAAAAAA | | AAGAAAUAAAAAAAA |
| STMN2_ | − | ATTT | 1126 | TCTTCTTAAACTAGA | 3132 | UCUUCUUAAACUAGA |
| intron1 | | | | GAAGAAATAAAAAAA | | GAAGAAAUAAAAAAA |
| STMN2_ | − | TTTC | 1127 | CTATTTTCTTCTTAA | 3133 | CUAUUUUCUUCUUAA |
| intron1 | | | | ACTAGAGAAGAAATA | | ACUAGAGAAGAAAUA |
| STMN2_ | − | CTTT | 1128 | CCTATTTTCTTCTTA | 3134 | CCUAUUUUCUUCUUA |
| intron1 | | | | AACTAGAGAAGAAAT | | AACUAGAGAAGAAAU |
| STMN2_ | − | TTTA | 1129 | CCCCTTTCCTATTTT | 3135 | CCCCUUUCCUAUUUU |
| intron1 | | | | CTTCTTAAACTAGAG | | CUUCUUAAACUAGAG |
| STMN2_ | − | CTTT | 1130 | ACCCCTTTCCTATTT | 3136 | ACCCCUUUCCUAUUU |
| intron1 | | | | TCTTCTTAAACTAGA | | UCUUCUUAAACUAGA |
| STMN2_ | − | CTTC | 1131 | CCTTTACCCCTTTCC | 3137 | CCUUUACCCCUUUCC |
| intron1 | | | | TATTTTCTTCTTAAA | | UAUUUUCUUCUUAAA |
| STMN2_ | − | TTTC | 1132 | TCCCACCTTCCCTTT | 3138 | UCCCACCUUCCCUUU |
| intron1 | | | | ACCCCTTTCCTATTT | | ACCCCUUUCCUAUUU |
| STMN2_ | − | CTTT | 1133 | CTCCCACCTTCCCTT | 3139 | CUCCCACCUUCCCUU |
| intron1 | | | | TACCCCTTTCCTATT | | UACCCCUUUCCUAUU |
| STMN2_ | − | TTTC | 1134 | CTTTCTCCCACCTTC | 3140 | CUUUCUCCCACCUUC |
| intron1 | | | | CCTTTACCCCTTTCC | | CCUUUACCCCUUUCC |
| STMN2_ | − | TTTT | 1135 | CCTTTCTCCCACCTT | 3141 | CCUUUCUCCCACCUU |
| intron1 | | | | CCCTTTACCCCTTTC | | CCCUUUACCCCUUUC |
| STMN2_ | − | TTTT | 1136 | TCCTTTCTCCCACCT | 3142 | UCCUUUCUCCCACCU |
| intron1 | | | | TCCCTTTACCCCTTT | | UCCCUUUACCCCUUU |
| STMN2_ | − | CTTT | 1137 | TTCCTTTCTCCCACC | 3143 | UUCCUUUCUCCCACC |
| intron1 | | | | TTCCCTTTACCCCTT | | UUCCCUUUACCCCUU |
| STMN2_ | − | TTTC | 1138 | TTTTTCCTTTCTCCC | 3144 | UUUUUCCUUUCUCCC |
| intron1 | | | | ACCTTCCCTTTACCC | | ACCUUCCCUUUACCC |
| STMN2_ | − | TTTT | 1139 | CTTTTTCCTTTCTCC | 3145 | CUUUUUCCUUUCUCC |
| intron1 | | | | CACCTTCCCTTTACC | | CACCUUCCCUUUACC |
| STMN2_ | − | ATTT | 1140 | TCTTTTTCCTTTCTC | 3146 | UCUUUUUCCUUUCUC |
| intron1 | | | | CCACCTTCCCTTTAC | | CCACCUUCCCUUUAC |
| STMN2_ | − | TTTG | 1141 | CAATTTTCTTTTTCC | 3147 | CAAUUUUCUUUUUCC |
| intron1 | | | | TTTCTCCCACCTTCC | | UUUCUCCCACCUUCC |
| STMN2_ | − | CTTT | 1142 | GCAATTTTCTTTTTC | 3148 | GCAAUUUUCUUUUUC |
| intron1 | | | | CTTTCTCCCACCTTC | | CUUUCUCCCACCUUC |
| STMN2_ | − | TTTG | 1143 | ACTTTGCAATTTTCT | 3149 | ACUUUGCAAUUUUCU |
| intron1 | | | | TTTTCCTTTCTCCCA | | UUUUCCUUUCUCCCA |
| STMN2_ | − | CTTT | 1144 | GACTTTGCAATTTTC | 3150 | GACUUUGCAAUUUUC |
| intron1 | | | | TTTTTCCTTTCTCCC | | UUUUUCCUUUCUCCC |
| STMN2_ | − | TTTC | 1145 | AAACAGCGGGATGGG | 3151 | AAACAGCGGGAUGGG |
| intron1 | | | | ACCGCTTTGACTTTG | | ACCGCUUUGACUUUG |
| STMN2_ | − | CTTA | 1146 | AGCCCATTTAAGGCA | 3152 | AGCCCAUUUAAGGCA |
| intron1 | | | | AACAGTTAAGGTAGC | | AACAGUUAAGGUAGC |
| STMN2_ | − | ATTT | 1147 | AAGGCAAACAGTTAA | 3153 | AAGGCAAACAGUUAA |
| intron1 | | | | | | |
| | | | | GGTAGCTTCCTCCCC | | GGUAGCUUCCUCCCC |
| STMN2_ | − | TTTA | 1148 | AGGCAAACAGTTAAG | 3154 | AGGCAAACAGUUAAG |
| intron1 | | | | GTAGCTTCCTCCCCT | | GUAGCUUCCUCCCCU |
| STMN2_ | − | GTTA | 1149 | AGGTAGCTTCCTCCC | 3155 | AGGUAGCUUCCUCCC |
| intron1 | | | | CTCACGATTGAGTCC | | CUCACGAUUGAGUCC |
| STMN2_ | − | CTTC | 1150 | TAGAGCTCAAGAGAG | 3156 | UAGAGCUCAAGAGAG |
| intron1 | | | | GAGGTGAGAGGTGGG | | GAGGUGAGAGGUGGG |
| STMN2_ | − | TTTA | 1151 | TAAAATATCTCTGAA | 3157 | UAAAAUAUCUCUGAA |
| intron1 | | | | TGCTTCTAGAGCTCA | | UGCUUCUAGAGCUCA |
| STMN2_ | − | CTTT | 1152 | ATAAAATATCTCTGA | 3158 | AUAAAAUAUCUCUGA |
| intron1 | | | | ATGCTTCTAGAGCTC | | AUGCUUCUAGAGCUC |
| STMN2_ | − | TTTC | 1153 | TTTATAAAATATCTC | 3159 | UUUAUAAAAUAUCUC |
| intron1 | | | | TGAATGCTTCTAGAG | | UGAAUGCUUCUAGAG |
| STMN2_ | − | TTTT | 1154 | CTTTATAAAATATCT | 3160 | CUUUAUAAAAUAUCU |
| intron1 | | | | CTGAATGCTTCTAGA | | CUGAAUGCUUCUAGA |
| STMN2_ | − | TTTT | 1155 | TCTTTATAAAATATC | 316 | UCUUUAUAAAAUAUC |
| intron1 | | | | TCTGAATGCTTCTAG | 1 | UCUGAAUGCUUCUAG |
| STMN2_ | − | CTTT | 1156 | TTCTTTATAAAATAT | 3162 | UUCUUUAUAAAAUAU |
| intron1 | | | | CTCTGAATGCTTCTA | | CUCUGAAUGCUUCUA |
| STMN2_ | − | ATTA | 1157 | ACATCTTTTTCTTTA | 3163 | ACAUCUUUUUCUUUA |
| intron1 | | | | TAAAATATCTCTGAA | | UAAAAUAUCUCUGAA |
| STMN2_ | − | GTTA | 1158 | CCATTAACATCTTTT | 3164 | CCAUUAACAUCUUUU |
| intron1 | | | | TCTTTATAAAATATC | | UCUUUAUAAAAUAUC |
| STMN2_ | − | CTTC | 1159 | CTGGTCCTGTGTTAC | 3165 | CUGGUCCUGUGUUAC |
| intron1 | | | | CATTAACATCTTTTT | | CAUUAACAUCUUUUU |
| STMN2_ | − | CTTC | 1160 | TCTGCCCTCCCACCT | 3166 | UCUGCCCUCCCACCU |
| intron1 | | | | CCCCCAGAACTGCCC | | CCCCCAGAACUGCCC |
| STMN2_ | − | TTTC | 1161 | CATAGACCTCTTCTC | 3167 | CAUAGACCUCUUCUC |
| intron1 | | | | TGCCCTCCCACCTCC | | UGCCCUCCCACCUCC |
| STMN2_ | − | ATTT | 1162 | CCATAGACCTCTTCT | 3168 | CCAUAGACCUCUUCU |
| intron1 | | | | CTGCCCTCCCACCTC | | CUGCCCUCCCACCUC |
| STMN2_ | − | CTTT | 1163 | CAAACAGCGGGATGG | 3169 | CAAACAGCGGGAUGG |
| intron1 | | | | GACCGCTTTGACTTT | | GACCGCUUUGACUUU |
| STMN2_ | − | TTTA | 1164 | GATTTCCATAGACCT | 3170 | GAUUUCCAUAGACCU |
| intron1 | | | | CTTCTCTGCCCTCCC | | CUUCUCUGCCCUCCC |
| STMN2_ | − | CTTC | 1165 | GCTTTAGATTTCCAT | 3171 | GCUUUAGAUUUCCAU |
| intron1 | | | | AGACCTCTTCTCTGC | | AGACCUCUUCUCUGC |
| STMN2_ | − | ATTC | 1166 | TTCGCTTTAGATTTC | 3172 | UUCGCUUUAGAUUUC |
| intron1 | | | | CATAGACCTCTTCTC | | CAUAGACCUCUUCUC |
| STMN2_ | − | TTTA | 1167 | AAAGAAATTCTTCGC | 3173 | AAAGAAAUUCUUCGC |
| intron1 | | | | TTTAGATTTCCATAG | | UUUAGAUUUCCAUAG |
| STMN2_ | − | TTTT | 1168 | AAAAGAAATTCTTCG | 3174 | AAAAGAAAUUCUUCG |
| intron1 | | | | CTTTAGATTTCCATA | | CUUUAGAUUUCCAUA |
| STMN2_ | − | CTTT | 1169 | TAAAAGAAATTCTTC | 3175 | UAAAAGAAAUUCUUC |
| intron1 | | | | GCTTTAGATTTCCAT | | GCUUUAGAUUUCCAU |
| STMN2_ | − | CTTC | 1170 | TACCTTTTAAAAGAA | 3176 | UACCUUUUAAAAGAA |
| intron1 | | | | ATTCTTCGCTTTAGA | | AUUCUUCGCUUUAGA |
| STMN2_ | − | CTTA | 1171 | CCCGCTTCTACCTTT | 3177 | CCCGCUUCUACCUUU |
| intron1 | | | | TAAAAGAAATTCTTC | | UAAAAGAAAUUCUUC |
| STMN2_ | − | ATTC | 1172 | TCTACCCATAGGAGG | 3178 | UCUACCCAUAGGAGG |
| intron1 | | | | GCAACTTACCCGCTT | | GCAACUUACCCGCUU |
| STMN2_ | − | TTTA | 1173 | AATATGGAAACAGAA | 3179 | AAUAUGGAAACAGAA |
| intron1 | | | | TAAATTCTCTACCCA | | UAAAUUCUCUACCCA |
| STMN2_ | − | TTTT | 1174 | AAATATGGAAACAGA | 3180 | AAAUAUGGAAACAGA |
| intron1 | | | | ATAAATTCTCTACCC | | AUAAAUUCUCUACCC |
| STMN2_ | − | ATTT | 1175 | TAAATATGGAAACAG | 3181 | UAAAUAUGGAAACAG |
| intron1 | | | | AATAAATTCTCTACC | | AAUAAAUUCUCUACC |
| STMN2_ | − | ATTG | 1176 | AGTCCTAATTTTAAA | 3182 | AGUCCUAAUUUUAAA |
| intron1 | | | | TATGGAAACAGAATA | | UAUGGAAACAGAAUA |
| STMN2_ | − | CTTC | 1177 | CTCCCCTCACGATTG | 3183 | CUCCCCUCACGAUUG |
| intron1 | | | | AGTCCTAATTTTAAA | | AGUCCUAAUUUUAAA |
| STMN2_ | − | CTTT | 1178 | AGATTTCCATAGACC | 3184 | AGAUUUCCAUAGACC |
| intron1 | | | | TCTTCTCTGCCCTCC | | UCUUCUCUGCCCUCC |
| STMN2_ | − | TTTC | 1179 | TTTCAGTTTTATCCT | 3185 | UUUCAGUUUUAUCCU |
| intron1 | | | | GAAATTCTGACCAAA | | GAAAUUCUGACCAAA |
| STMN2_ | − | GTTC | 1180 | TCTCCATCCCCTCCC | 3186 | UCUCCAUCCCCUCCC |
| intron1 | | | | CCCGTCTCCACCCAT | | CCCGUCUCCACCCAU |
| STMN2_ | − | TTTC | 1181 | TTCGACGAGACAATA | 3187 | UUCGACGAGACAAUA |
| intron1 | | | | CCGTAAAATGTGCCC | | CCGUAAAAUGUGCCC |
| STMN2_ | − | TTTA | 1182 | TATACGATTTCATGT | 3188 | UAUACGAUUUCAUGU |
| intron1 | | | | CATCTCTATTATTAT | | |
| | | | | | | CAUCUCUAUUAUUAU |
| STMN2_ | − | CTTT | 1183 | ATATACGATTTCATG | 3189 | AUAUACGAUUUCAUG |
| intron1 | | | | TCATCTCTATTATTA | | UCAUCUCUAUUAUUA |
| STMN2_ | − | TTTG | 1184 | CTTTATATACGATTT | 3190 | CUUUAUAUACGAUUU |
| intron1 | | | | CATGTCATCTCTATT | | CAUGUCAUCUCUAUU |
| STMN2_ | − | TTTT | 1185 | GCTTTATATACGATT | 3191 | GCUUUAUAUACGAUU |
| intron1 | | | | TCATGTCATCTCTAT | | UCAUGUCAUCUCUAU |
| STMN2_ | − | CTTT | 1186 | TGCTTTATATACGAT | 3192 | UGCUUUAUAUACGAU |
| intron1 | | | | TTCATGTCATCTCTA | | UUCAUGUCAUCUCUA |
| STMN2_ | − | TTTG | 1187 | ACCTCTTTTGCTTTA | 3193 | ACCUCUUUUGCUUUA |
| intron1 | | | | TATACGATTTCATGT | | UAUACGAUUUCAUGU |
| STMN2_ | − | CTTT | 1188 | GACCTCTTTTGCTTT | 3194 | GACCUCUUUUGCUUU |
| intron1 | | | | ATATACGATTTCATG | | AUAUACGAUUUCAUG |
| STMN2_ | − | CTTA | 1189 | AGACTTTGACCTCTT | 3195 | AGACUUUGACCUCUU |
| intron1 | | | | TTGCTTTATATACGA | | UUGCUUUAUAUACGA |
| STMN2_ | − | CTTA | 1190 | ACTTAAGACTTTGAC | 3196 | ACUUAAGACUUUGAC |
| intron1 | | | | CTCTTTTGCTTTATA | | CUCUUUUGCUUUAUA |
| STMN2_ | − | TTTC | 1191 | GCGTGGCTTAACTTA | 3197 | GCGUGGCUUAACUUA |
| intron1 | | | | AGACTTTGACCTCTT | | AGACUUUGACCUCUU |
| STMN2_ | − | ATTT | 1192 | CGCGTGGCTTAACTT | 3198 | CGCGUGGCUUAACUU |
| intron1 | | | | AAGACTTTGACCTCT | | AAGACUUUGACCUCU |
| STMN2_ | − | TTTG | 1193 | GCACTGTCTGACCCA | 3199 | GCACUGUCUGACCCA |
| intron1 | | | | CAAAACGGAAATTTC | | CAAAACGGAAAUUUC |
| STMN2_ | − | ATTT | 1194 | GGCACTGTCTGACCC | 3200 | GGCACUGUCUGACCC |
| intron1 | | | | ACAAAACGGAAATTT | | ACAAAACGGAAAUUU |
| STMN2_ | − | ATTG | 1195 | CCGATATTTGGCACT | 3201 | CCGAUAUUUGGCACU |
| intron1 | | | | GTCTGACCCACAAAA | | GUCUGACCCACAAAA |
| STMN2_ | − | CTTA | 1196 | TGAAATTGCCGATAT | 3202 | UGAAAUUGCCGAUAU |
| intron1 | | | | TTGGCACTGTCTGAC | | UUGGCACUGUCUGAC |
| STMN2_ | − | CTTG | 1197 | TCTCTCTGAGCTTAT | 3203 | UCUCUCUGAGCUUAU |
| intron1 | | | | GAAATTGCCGATATT | | GAAAUUGCCGAUAUU |
| STMN2_ | − | TTTC | 1198 | CGGTCATCCTGTGTC | 3204 | CGGUCAUCCUGUGUC |
| intron1 | | | | TCCACTGTCTTGTCT | | UCCACUGUCUUGUCU |
| STMN2_ | − | TTTT | 1199 | CCGGTCATCCTGTGT | 3205 | CCGGUCAUCCUGUGU |
| intron1 | | | | CTCCACTGTCTTGTC | | CUCCACUGUCUUGUC |
| STMN2_ | − | CTTT | 1200 | TCCGGTCATCCTGTG | 3206 | UCCGGUCAUCCUGUG |
| intron1 | | | | TCTCCACTGTCTTGT | | UCUCCACUGUCUUGU |
| STMN2_ | − | ATTG | 1201 | CGGATGAAGGCCCTG | 3207 | CGGAUGAAGGCCCUG |
| intron1 | | | | AATCCAGAATCTTTT | | AAUCCAGAAUCUUUU |
| STMN2_ | − | TTTC | 1202 | ACCCCGGGGCCACTG | 3208 | ACCCCGGGGCCACUG |
| intron1 | | | | AGCGCCAGAACCGTG | | AGCGCCAGAACCGUG |
| STMN2_ | − | TTTT | 1203 | CACCCCGGGGCCACT | 3209 | CACCCCGGGGCCACU |
| intron1 | | | | GAGCGCCAGAACCGT | | GAGCGCCAGAACCGU |
| STMN2_ | − | CTTT | 1204 | TCACCCCGGGGCCAC | 3210 | UCACCCCGGGGCCAC |
| intron1 | | | | TGAGCGCCAGAACCG | | UGAGCGCCAGAACCG |
| STMN2_ | − | CTTC | 1205 | CAGCTGCCACAGGAC | 3211 | CAGCUGCCACAGGAC |
| intron1 | | | | CCCAGGCCCCACCCT | | CCCAGGCCCCACCCU |
| STMN2_ | − | TTTC | 1206 | CCTGTCTCCGCATCC | 3212 | CCUGUCUCCGCAUCC |
| intron1 | | | | TGCAACCAAGTCCCG | | UGCAACCAAGUCCCG |
| STMN2_ | − | CTTT | 1207 | CCCTGTCTCCGCATC | 3213 | CCCUGUCUCCGCAUC |
| intron1 | | | | CTGCAACCAAGTCCC | | CUGCAACCAAGUCCC |
| STMN2_ | − | TTTC | 1208 | GGCAGCTTTCCCTGT | 3214 | GGCAGCUUUCCCUGU |
| intron1 | | | | CTCCGCATCCTGCAA | | CUCCGCAUCCUGCAA |
| STMN2_ | − | ATTT | 1209 | CATGTCATCTCTATT | 3215 | CAUGUCAUCUCUAUU |
| intron1 | | | | ATTATACATACACAT | | AUUAUACAUACACAU |
| STMN2_ | − | TTTC | 1210 | ATGTCATCTCTATTA | 3216 | AUGUCAUCUCUAUUA |
| intron1 | | | | TTATACATACACATG | | UUAUACAUACACAUG |
| STMN2_ | − | ATTA | 1211 | TTATACATACACATG | 3217 | UUAUACAUACACAUG |
| intron1 | | | | TCTAGGTTCTAGAAG | | UCUAGGUUCUAGAAG |
| STMN2_ | − | ATTA | 1212 | TACATACACATGTCT | 3218 | UACAUACACAUGUCU |
| intron1 | | | | AGGTTCTAGAAGCTT | | AGGUUCUAGAAGCUU |
| STMN2_ | − | GTTT | 1213 | CTTCGACGAGACAAT | 3219 | CUUCGACGAGACAAU |
| intron1 | | | | ACCGTAAAATGTGCC | | ACCGUAAAAUGUGCC |
| STMN2_ | − | CTTA | 1214 | CCTCCCTGCACCGCA | 3220 | CCUCCCUGCACCGCA |
| intron1 | | | | CCCCAGGACTAGCGG | | CCCCAGGACUAGCGG |
| STMN2_ | | CTTG | 1215 | CCCTAAAACAAAGGA | 3221 | CCCUAAAACAAAGGA |
| intron1 | | | | GCGGAGGTCCTACCC | | GCGGAGGUCCUACCC |
| STMN2_ | − | CTTC | 1216 | CCCTCCCTTGCCCTA | 3222 | CCCUCCCUUGCCCUA |
| intron1 | | | | AAACAAAGGAGCGGA | | AAACAAAGGAGCGGA |
| STMN2_ | − | CTTC | 1217 | CTCTCTCCTTCCCCT | 3223 | CUCUCUCCUUCCCCU |
| intron1 | | | | CCCTTGCCCTAAAAC | | CCCUUGCCCUAAAAC |
| STMN2_ | − | CTTC | 1218 | CCCGCCCCTGCAGCT | 3224 | CCCGCCCCUGCAGCU |
| intron1 | | | | GCCCACCCGCGCCCT | | GCCCACCCGCGCCCU |
| STMN2_ | − | CTTC | 1219 | GAAGCCGCTGTCCCT | 3225 | GAAGCCGCUGUCCCU |
| intron1 | | | | CCACCCCTCCCTGCC | | CCACCCCUCCCUGCC |
| STMN2_ | − | ATTG | 1220 | TGCGCCCAGCGCTGC | 3226 | UGCGCCCAGCGCUGC |
| intron1 | | | | AGGTGCCTCCCCCCG | | AGGUGCCUCCCCCCG |
| STMN2_ | − | GTTC | 1221 | CGCACTGGGTGGGGC | 3227 | CGCACUGGGUGGGGC |
| intron1 | | | | TGTCCGCATTGTGCG | | UGUCCGCAUUGUGCG |
| STMN2_ | − | TTTC | 1222 | GAATGAAGATGCAGC | 3228 | GAAUGAAGAUGCAGC |
| intron1 | | | | ACCGGGGGGGGGGGG | | ACCGGGGGGGGGGGG |
| STMN2_ | − | CTTT | 1223 | CGAATGAAGATGCAG | 3229 | CGAAUGAAGAUGCAG |
| intron1 | | | | CACCGGGCGGGGGGG | | CACCGGGCGGGGGGG |
| STMN2_ | − | GTTG | 1224 | GGCTCCTGGGTGTCA | 3230 | GGCUCCUGGGUGUCA |
| intron1 | | | | CGCTGCGCTCCCCAC | | CGCUGCGCUCCCCAC |
| STMN2_ | − | CTTG | 1225 | GAAGCCGCGGCGGGG | 3231 | GAAGCCGCGGCGGGG |
| intron1 | | | | AGTCGGGAGCGGGGA | | AGUCGGGAGCGGGGA |
| STMN2_ | − | CTTC | 1226 | GACGAGACAATACCG | 3232 | GACGAGACAAUACCG |
| intron1 | | | | TAAAATGTGCCCAGT | | UAAAAUGUGCCCAGU |
| STMN2_ | − | CTTA | 1227 | AAAGCAGAACAATGA | 3233 | AAAGCAGAACAAUGA |
| intron1 | | | | GGCCAGCGTGGGGAG | | GGCCAGCGUGGGGAG |
| STMN2_ | − | TTTT | 1228 | CCCATCTCTCTTAAA | 3234 | CCCAUCUCUCUUAAA |
| intron1 | | | | AGCAGAACAATGAGG | | AGCAGAACAAUGAGG |
| STMN2_ | − | CTTT | 1229 | TCCCATCTCTCTTAA | 3235 | UCCCAUCUCUCUUAA |
| intron1 | | | | AAGCAGAACAATGAG | | AAGCAGAACAAUGAG |
| STMN2_ | − | GTTA | 1230 | ACCCACTTTTCCCAT | 3236 | ACCCACUUUUCCCAU |
| intron1 | | | | CTCTCTTAAAAGCAG | | CUCUCUUAAAAGCAG |
| STMN2_ | − | CTTC | 1231 | CGAAAAGAAAAATGT | 3237 | CGAAAAGAAAAAUGU |
| intron1 | | | | TAACCCACTTTTCCC | | UAACCCACUUUUCCC |
| STMN2_ | − | TTTG | 1232 | CTTCCGAAAAGAAAA | 3238 | CUUCCGAAAAGAAAA |
| intron1 | | | | ATGTTAACCCACTTT | | AUGUUAACCCACUUU |
| STMN2_ | − | ATTT | 1233 | GCTTCCGAAAAGAAA | 3239 | GCUUCCGAAAAGAAA |
| intron1 | | | | AATGTTAACCCACTT | | AAUGUUAACCCACUU |
| STMN2_ | | TTTA | 1234 | TCTGTGTCTATGTCT | 3240 | UCUGUGUCUAUGUCU |
| intron1 | | | | AAACACTCTATGTAA | | AAACACUCUAUGUAA |
| STMN2_ | − | CTTT | 1235 | ATGTGTGTCTATGTC | 3241 | AUCUGUGUCUAUGUC |
| intron1 | | | | TAAACACTCTATGTA | | UAAACACUCUAUGUA |
| STMN2_ | − | CTTC | 1236 | AAAGAACCCTTTATC | 3242 | AAAGAACCCUUUAUC |
| intron1 | | | | TGTGTCTATGTCTAA | | UGUGUCUAUGUCUAA |
| STMN2_ | − | TTTC | 1237 | CCGCAAACGATCAAA | 3243 | CCGCAAACGAUCAAA |
| intron1 | | | | GGTCTTCAAAGAACC | | GGUCUUCAAAGAACC |
| STMN2_ | − | TTTT | 1238 | CCCGCAAACGATCAA | 3244 | CCCGCAAACGAUCAA |
| intron1 | | | | AGGTCTTCAAAGAAC | | AGGUCUUCAAAGAAC |
| STMN2_ | − | CTTT | 1239 | TCCCGCAAACGATCA | 3245 | UCCCGCAAACGAUCA |
| intron1 | | | | AAGGTCTTCAAAGAA | | AAGGUCUUCAAAGAA |
| STMN2_ | − | GTTC | 1240 | TAGAAGCTTTTCCCG | 3246 | UAGAAGCUUUUCCCG |
| intron1 | | | | CAAACGATCAAAGGT | | CAAACGAUCAAAGGU |
| STMN2_ | − | TTTC | 1241 | CCATCTCTCTTAAAA | 3247 | CCAUCUCUCUUAAAA |
| intron1 | | | | GCAGAACAATGAGGC | | GCAGAACAAUGAGGC |
| STMN2_ | − | ATTT | 1242 | TGTAAAGATTACCAT | 3248 | UGUAAAGAUUACCAU |
| intron1 | | | | AGATTTAAAAATGTT | | AGAUUUAAAAAUGUU |
| STMN2_ | − | ATTT | 1243 | CTTTCAGTTTTATCC | 3249 | CUUUCAGUUUUAUCC |
| intron1 | | | | TGAAATTCTGACCAA | | UGAAAUUCUGACCAA |
| STMN2_ | − | ATTA | 1244 | ATTGATAAACTACTG | 3250 | AUUGAUAAACUACUG |
| intron1 | | | | CCATTTCTTTCAGTT | | CCAUUUCUUUCAGUU |
| STMN2_ | − | TTTC | 1245 | TACTATTTATCCACT | 3251 | UACUAUUUAUCCACU |
| intron1 | | | | ACAAAATCTCAGAAG | | ACAAAAUCUCAGAAG |
| STMN2_ | − | TTTT | 1246 | CTACTATTTATCCAC | 3252 | CUACUAUUUAUCCAC |
| intron1 | | | | TACAAAATCTCAGAA | | UACAAAAUCUCAGAA |
| STMN2_ | − | TTTT | 1247 | TCTACTATTTATCCA | 3253 | UCUACUAUUUAUCCA |
| intron1 | | | | CTACAAAATCTCAGA | | CUACAAAAUCUCAGA |
| STMN2_ | − | ATTT | 1248 | TTCTACTATTTATCC | 3254 | UUCUACUAUUUAUCC |
| intron1 | | | | ACTACAAAATCTCAG | | ACUACAAAAUCUCAG |
| STMN2_ | − | ATTA | 1249 | CTACTGACATTTTTC | 3255 | CUACUGACAUUUUUC |
| intron1 | | | | TACTATTTATCCACT | | UACUAUUUAUCCACU |
| STMN2_ | − | TTTG | 1250 | CTATTACTACTGACA | 3256 | CUAUUACUACUGACA |
| intron1 | | | | TTTTTCTACTATTTA | | UUUUUCUACUAUUUA |
| STMN2_ | − | CTTT | 1251 | GCTATTACTACTGAC | 3257 | GCUAUUACUACUGAC |
| intron1 | | | | ATTTTTCTACTATTT | | AUUUUUCUACUAUUU |
| STMN2_ | − | ATTC | 1252 | GGCTGCTAAATAACT | 3258 | GGCUGCUAAAUAACU |
| intron1 | | | | TTGCTATTACTACTG | | UUGCUAUUACUACUG |
| STMN2_ | − | ATTA | 1253 | AAATATTCGGCTGCT | 3259 | AAAUAUUCGGCUGCU |
| intron1 | | | | AAATAACTTTGCTAT | | AAAUAACUUUGCUAU |
| STMN2_ | − | TTTA | 1254 | AGCATTAAAATATTC | 3260 | AGCAUUAAAAUAUUC |
| intron1 | | | | GGCTGCTAAATAACT | | GGCUGCUAAAUAACU |
| STMN2_ | − | TTTT | 1255 | AAGCATTAAAATATT | 3261 | AAGCAUUAAAAUAUU |
| intron1 | | | | CGGCTGCTAAATAAC | | CGGCUGCUAAAUAAC |
| STMN2_ | − | TTTT | 1256 | TAAGCATTAAAATAT | 3262 | UAAGCAUUAAAAUAU |
| intron1 | | | | TCGGCTGCTAAATAA | | UCGGCUGCUAAAUAA |
| STMN2_ | − | ATTT | 1257 | TTAAGCATTAAAATA | 3263 | UUAAGCAUUAAAAUA |
| intron1 | | | | TTCGGCTGCTAAATA | | UUCGGCUGCUAAAUA |
| STMN2_ | − | TTTA | 1258 | TTTTTAAGCATTAAA | 3264 | UUUUUAAGCAUUAAA |
| intron1 | | | | ATATTCGGCTGCTAA | | AUAUUCGGCUGCUAA |
| STMN2_ | − | CTTT | 1259 | ATTTTTAAGCATTAA | 3265 | AUUUUUAAGCAUUAA |
| intron1 | | | | AATATTCGGCTGCTA | | AAUAUUCGGCUGCUA |
| STMN2_ | − | ATTC | 1260 | CTTTATTTTTAAGCA | 3266 | CUUUAUUUUUAAGCA |
| intron1 | | | | TTAAAATATTCGGCT | | UUAAAAUAUUCGGCU |
| STMN2_ | − | TTTA | 1261 | TTCCTTTATTTTTAA | 3267 | UUCCUUUAUUUUUAA |
| intron1 | | | | GCATTAAAATATTCG | | GCAUUAAAAUAUUCG |
| STMN2_ | − | ATTT | 1262 | ATTCCTTTATTTTTA | 3268 | AUUCCUUUAUUUUUA |
| intron1 | | | | AGCATTAAAATATTC | | AGCAUUAAAAUAUUC |
| STMN2_ | − | TTTA | 1263 | ATTTATTCCTTTATT | 3269 | AUUUAUUCCUUUAUU |
| intron1 | | | | TTTAAGCATTAAAAT | | UUUAAGCAUUAAAAU |
| STMN2_ | − | CTTT | 1264 | AATTTATTCCTTTAT | 3270 | AAUUUAUUCCUUUAU |
| intron1 | | | | TTTTAAGCATTAAAA | | UUUUAAGCAUUAAAA |
| STMN2_ | − | TTTC | 1265 | TTTAATTTATTCCTT | 3271 | UUUAAUUUAUUCCUU |
| intron1 | | | | TATTTTTAAGCATTA | | UAUUUUUAAGCAUUA |
| STMN2_ | − | TTTT | 1266 | CTTTAATTTATTCCT | 3272 | CUUUAAUUUAUUCCU |
| intron1 | | | | TTATTTTTAAGCATT | | UUAUUUUUAAGCAUU |
| STMN2_ | − | ATTT | 1267 | TCTTTAATTTATTCC | 3273 | UCUUUAAUUUAUUCC |
| intron1 | | | | TTTATTTTTAAGCAT | | UUUAUUUUUAAGCAU |
| STMN2_ | − | ATTT | 1268 | CAATCGATGAAGAAG | 3274 | CAAUCGAUGAAGAAG |
| intron1 | | | | TAAACAATGATTTTC | | UAAACAAUGAUUUUC |
| STMN2_ | − | ATTC | 1269 | AGATGTGCTCTGAAC | 3275 | AGAUGUGCUCUGAAC |
| intron1 | | | | AGGGGGCACATTTCA | | AGGGGGCACAUUUCA |
| STMN2_ | − | GTTC | 1270 | TCTGCAGGTGGAGAC | 3276 | UCUGCAGGUGGAGAC |
| intron1 | | | | TCTGATATTCAGATG | | UCUGAUAUUCAGAUG |
| STMN2_ | − | TTTA | 1271 | CTCGCTAAGCTGCAT | 3277 | CUCGCUAAGCUGCAU |
| intron1 | | | | GTTCTCTGCAGGTGG | | GUUCUCUGCAGGUGG |
| STMN2_ | − | ATTT | 1272 | ATCCACTACAAAATC | 3278 | AUCCACUACAAAAUC |
| intron1 | | | | TCAGAAGTAACATAA | | UCAGAAGUAACAUAA |
| STMN2_ | − | TTTT | 1273 | ACTCGCTAAGCTGCA | 3279 | ACUCGCUAAGCUGCA |
| intron1 | | | | TGTTCTCTGCAGGTG | | UGUUCUCUGCAGGUG |
| STMN2_ | − | TTTA | 1274 | TCCACTACAAAATCT | 3280 | UCCACUACAAAAUCU |
| intron1 | | | | CAGAAGTAACATAAA | | CAGAAGUAACAUAAA |
| STMN2_ | − | ATTA | 1275 | ACCAGGGCGTGTATC | 3281 | ACCAGGGCGUGUAUC |
| intron1 | | | | TACTTTCAGATTATG | | UACUUUCAGAUUAUG |
| STMN2_ | − | ATTG | 1276 | CCCTCTAGTGTGGTG | 3282 | CCCUCUAGUGUGGUG |
| intron1 | | | | AAAAGTTAATGCAGA | | AAAAGUUAAUGCAGA |
| STMN2_ | − | TTTA | 1277 | GAGAACATGATTGCC | 3283 | GAGAACAUGAUUGCC |
| intron1 | | | | CTCTAGTGTGGTGAA | | CUCUAGUGUGGUGAA |
| STMN2_ | − | TTTT | 1278 | AGAGAACATGATTGC | 3284 | AGAGAACAUGAUUGC |
| intron1 | | | | CCTCTAGTGTGGTGA | | CCUCUAGUGUGGUGA |
| STMN2_ | − | TTTT | 1279 | TAGAGAACATGATTG | 3285 | UAGAGAACAUGAUUG |
| intron1 | | | | CCCTCTAGTGTGGTG | | CCCUCUAGUGUGGUG |
| STMN2_ | − | TTTT | 1280 | TTAGAGAACATGATT | 3286 | UUAGAGAACAUGAUU |
| intron1 | | | | GCCCTCTAGTGTGGT | | GCCCUCUAGUGUGGU |
| STMN2_ | − | CTTT | 1281 | TTTAGAGAACATGAT | 3287 | UUUAGAGAACAUGAU |
| intron1 | | | | TGCCCTCTAGTGTGG | | UGCCCUCUAGUGUGG |
| STMN2_ | − | TTTA | 1282 | CATCAATCATCTGCT | 3288 | CAUCAAUCAUCUGCU |
| intron1 | | | | TTTTTAGAGAACATG | | UUUUUAGAGAACAUG |
| STMN2_ | − | GTTT | 1283 | ACATCAATCATCTGC | 3289 | ACAUCAAUCAUCUGC |
| intron1 | | | | TTTTTTAGAGAACAT | | UUUUUUAGAGAACAU |
| STMN2_ | − | TTTG | 1284 | GAACTAGGTTTACAT | 3290 | GAACUAGGUUUACAU |
| intron1 | | | | CAATCATCTGCTTTT | | CAAUCAUCUGCUUUU |
| STMN2_ | − | ATTT | 1285 | GGAACTAGGTTTACA | 3291 | GGAACUAGGUUUACA |
| intron1 | | | | TCAATCATCTGCTTT | | UCAAUCAUCUGCUUU |
| STMN2_ | − | GTTA | 1286 | ATATTTGGAACTAGG | 3292 | AUAUUUGGAACUAGG |
| intron1 | | | | TTTACATCAATCATC | | UUUACAUCAAUCAUC |
| STMN2_ | − | ATTA | 1287 | AACAGTTAATATTTG | 3293 | AACAGUUAAUAUUUG |
| intron1 | | | | GAACTAGGTTTACAT | | GAACUAGGUUUACAU |
| STMN2_ | − | TTTA | 1288 | TTAAACAGTTAATAT | 3294 | UUAAACAGUUAAUAU |
| intron1 | | | | TTGGAACTAGGTTTA | | UUGGAACUAGGUUUA |
| STMN2_ | − | TTTT | 1289 | ATTAAACAGTTAATA | 3295 | AUUAAACAGUUAAUA |
| intron1 | | | | TTTGGAACTAGGTTT | | UUUGGAACUAGGUUU |
| STMN2_ | − | ATTT | 1290 | TATTAAACAGTTAAT | 3296 | UAUUAAACAGUUAAU |
| intron1 | | | | ATTTGGAACTAGGTT | | AUUUGGAACUAGGUU |
| STMN2_ | − | GTTC | 1291 | CTGGTAAAAGAAAAG | 329 | CUGGUAAAAGAAAAG |
| intron1 | | | | ATTTTATTAAACAGT | 7 | AUUUUAUUAAACAGU |
| STMN2_ | − | CTTG | 1292 | AATGTTCCTGGTAAA | 3298 | AAUGUUCCUGGUAAA |
| intron1 | | | | AGAAAAGATTTTATT | | AGAAAAGAUUUUAUU |
| STMN2_ | − | ATTG | 1293 | AATAAACACTTGAAT | 3299 | AAUAAACACUUGAAU |
| intron1 | | | | GTTCCTGGTAAAAGA | | GUUCCUGGUAAAAGA |
| STMN2_ | − | CTTA | 1294 | TTGAATAAACACTTG | 3300 | UUGAAUAAACACUUG |
| intron1 | | | | AATGTTCCTGGTAAA | | AAUGUUCCUGGUAAA |
| STMN2_ | − | GTTC | 1295 | ATCCACTAGGGTAAA | 3301 | AUCCACUAGGGUAAA |
| intron1 | | | | GCATGGCATCAGCTT | | GCAUGGCAUCAGCUU |
| STMN2_ | − | ATTG | 1296 | TACAAGCTCTGTTCA | 3302 | UACAAGCUCUGUUCA |
| intron1 | | | | TCCACTAGGGTAAAG | | UCCACUAGGGUAAAG |
| STMN2_ | − | CTTG | 1297 | AAAATTGTACAAGCT | 3303 | AAAAUUGUACAAGCU |
| intron1 | | | | CTGTTCATCCACTAG | | CUGUUCAUCCACUAG |
| STMN2_ | − | TTTC | 1298 | ATCCTGTCTCCTTGA | 3304 | AUCCUGUCUCCUUGA |
| intron1 | | | | AAATTGTACAAGCTC | | AAAUUGUACAAGCUC |
| STMN2_ | − | ATTT | 1299 | CATCCTGTCTCCTTG | 3305 | CAUCCUGUCUCCUUG |
| intron1 | | | | AAAATTGTACAAGCT | | AAAAUUGUACAAGCU |
| STMN2_ | − | ATTA | 1300 | TGACCACTCATTTCA | 3306 | UGACCACUCAUUUCA |
| intron1 | | | | TCCTGTCTCCTTGAA | | UCCUGUCUCCUUGAA |
| STMN2_ | − | TTTC | 1301 | AGATTATGACCACTC | 3307 | AGAUUAUGACCACUC |
| intron1 | | | | ATTTCATCCTGTCTC | | AUUUCAUCCUGUCUC |
| STMN2_ | − | CTTT | 1302 | CAGATTATGACCACT | 3308 | CAGAUUAUGACCACU |
| intron1 | | | | CATTTCATCCTGTCT | | CAUUUCAUCCUGUCU |
| STMN2_ | − | ATTA | 1303 | TAATAACAATGTAAT | 3309 | UAAUAACAAUGUAAU |
| intron1 | | | | AAAACTGAGAAGTAA | | AAAACUGAGAAGUAA |
| STMN2_ | − | GTTT | 1304 | TACTCGCTAAGCTGC | 3310 | UACUCGCUAAGCUGC |
| intron1 | | | | ATGTTCTCTGCAGGT | | AUGUUCUCUGCAGGU |
| STMN2_ | − | TTTG | 1305 | GTACACCTCCTCAGT | 3311 | GUACACCUCCUCAGU |
| intron1 | | | | ATCACATACCTGCCT | | AUCACAUACCUGCCU |
| STMN2_ | − | TTTT | 1306 | GGTACACCTCCTCAG | 3312 | GGUACACCUCCUCAG |
| intron1 | | | | TATCACATACCTGCC | | UAUCACAUACCUGCC |
| STMN2_ | − | ATTA | 1307 | CATAAAATGTAATCA | 3313 | CAUAAAAUGUAAUCA |
| intron1 | | | | AAAAATAATTCTATC | | AAAAAUAAUUCUAUC |
| STMN2_ | − | ATTA | 1308 | GAATTACATAAAATG | 3314 | GAAUUACAUAAAAUG |
| intron1 | | | | TAATCAAAAAATAAT | | UAAUCAAAAAAUAAU |
| STMN2_ | − | TTTA | 1309 | TAGCTGGATTAGAAT | 3315 | UAGCUGGAUUAGAAU |
| intron1 | | | | TACATAAAATGTAAT | | UACAUAAAAUGUAAU |
| STMN2_ | − | TTTT | 1310 | ATAGCTGGATTAGAA | 3316 | AUAGCUGGAUUAGAA |
| intron1 | | | | TTACATAAAATGTAA | | UUACAUAAAAUGUAA |
| STMN2_ | − | ATTT | 1311 | TATAGCTGGATTAGA | 3317 | UAUAGCUGGAUUAGA |
| intron1 | | | | ATTACATAAAATGTA | | AUUACAUAAAAUGUA |
| STMN2_ | − | ATTA | 1312 | AATATTTTATAGCTG | 3318 | AAUAUUUUAUAGCUG |
| intron1 | | | | GATTAGAATTACATA | | GAUUAGAAUUACAUA |
| STMN2_ | − | TTTG | 1313 | AGGAACACAGTAATA | 3319 | AGGAACACAGUAAUA |
| intron1 | | | | TGACACTATTAAATA | | UGACACUAUUAAAUA |
| STMN2_ | − | GTTT | 1314 | GAGGAACACAGTAAT | 3320 | GAGGAACACAGUAAU |
| intron1 | | | | ATGACACTATTAAAT | | AUGACACUAUUAAAU |
| STMN2_ | − | ATTC | 1315 | ATATGCACATCAAAG | 3321 | AUAUGCACAUCAAAG |
| intron1 | | | | TTTGAGGAACACAGT | | UUUGAGGAACACAGU |
| STMN2_ | − | TTTA | 1316 | ATGAAAATCAAAGGT | 3322 | AUGAAAAUCAAAGGU |
| intron1 | | | | AATTCATATGCACAT | | AAUUCAUAUGCACAU |
| STMN2_ | − | TTTT | 1317 | AATGAAAATCAAAGG | 3323 | AAUGAAAAUCAAAGG |
| intron1 | | | | TAATTCATATGCACA | | UAAUUCAUAUGCACA |
| STMN2_ | − | ATTT | 1318 | TAATGAAAATCAAAG | 3324 | UAAUGAAAAUCAAAG |
| intron1 | | | | GTAATTCATATGCAC | | GUAAUUCAUAUGCAC |
| STMN2_ | − | TTTG | 1319 | CATTTTAATGAAAAT | 3325 | CAUUUUAAUGAAAAU |
| intron1 | | | | CAAAGGTAATTCATA | | CAAAGGUAAUUCAUA |
| STMN2_ | − | ATTT | 1320 | GCATTTTAATGAAAA | 3326 | GCAUUUUAAUGAAAA |
| intron1 | | | | TCAAAGGTAATTCAT | | UCAAAGGUAAUUCAU |
| STMN2_ | − | ATTG | 1321 | AATCAGAATTTGCAT | 3327 | AAUCAGAAUUUGCAU |
| intron1 | | | | TTTAATGAAAATCAA | | UUUAAUGAAAAUCAA |
| STMN2_ | − | GTTC | 1322 | GGAAGACAGAATGTC | 3328 | GGAAGACAGAAUGUC |
| intron1 | | | | TGCCTCAAGCCAGAT | | UGCCUCAAGCCAGAU |
| STMN2_ | − | CTTG | 1323 | TTCGGAAGACAGAAT | 3329 | UUCGGAAGACAGAAU |
| intron1 | | | | GTCTGCCTCAAGCCA | | GUCUGCCUCAAGCCA |
| STMN2_ | − | TTTA | 1324 | GTGGTCAGAATCAGC | 3330 | GUGGUCAGAAUCAGC |
| intron1 | | | | ATCATCTGGGAGCTT | | AUCAUCUGGGAGCUU |
| STMN2_ | − | GTTT | 1325 | AGTGGTCAGAATCAG | 3331 | AGUGGUCAGAAUCAG |
| intron1 | | | | CATCATCTGGGAGCT | | CAUCAUCUGGGAGCU |
| STMN2_ | − | GTTA | 1326 | ATATCCCTAAAACTG | 3332 | AUAUCCCUAAAACUG |
| intron1 | | | | ATGTGTTTAGTGGTC | | AUGUGUUUAGUGGUC |
| STMN2_ | − | ATTA | 1327 | CAAGTTAATATCCCT | 333 | CAAGUUAAUAUCCCU |
| intron1 | | | | AAAACTGATGTGTTT | 3 | AAAACUGAUGUGUUU |
| STMN2_ | | CTTA | 1328 | CCAGGAGGGATACCT | 3334 | CCAGGAGGGAUACCU |
| intron1 | | | | GTATATTACAAGTTA | | GUAUAUUACAAGUUA |
| STMN2_ | − | GTTA | 1329 | AGACATAATACCAGA | 3335 | AGACAUAAUACCAGA |
| intron1 | | | | GCTTACCAGGAGGGA | | GCUUACCAGGAGGGA |
| STMN2_ | − | TTTA | 1330 | AAAATGTTAAGACAT | 3336 | AAAAUGUUAAGACAU |
| intron1 | | | | AATACCAGAGCTTAC | | AAUACCAGAGCUUAC |
| STMN2_ | − | ATTT | 1331 | AAAAATGTTAAGACA | 3337 | AAAAAUGUUAAGACA |
| intron1 | | | | TAATACCAGAGCTTA | | UAAUACCAGAGCUUA |
| STMN2_ | − | ATTA | 1332 | CCATAGATTTAAAAA | 3338 | CCAUAGAUUUAAAAA |
| intron1 | | | | TGTTAAGACATAATA | | UGUUAAGACAUAAUA |
| STMN2_ | − | TTTG | 1333 | TAAAGATTACCATAG | 3339 | UAAAGAUUACCAUAG |
| intron1 | | | | ATTTAAAAATGTTAA | | AUUUAAAAAUGUUAA |
| STMN2_ | − | ATTC | 1334 | TATCAATGCATATTT | 3340 | UAUCAAUGCAUAUUU |
| intron1 | | | | AAAAAATCCACTTTT | | AAAAAAUCCACUUUU |
| STMN2_ | − | ATTT | 1335 | AAAAAATCCACTTTT | 3341 | AAAAAAUCCACUUUU |
| intron1 | | | | GATGATACCCAAAAT | | GAUGAUACCCAAAAU |
| STMN2_ | − | TTTA | 1336 | AAAAATCCACTTTTG | 3342 | AAAAAUCCACUUUUG |
| intron1 | | | | ATGATACCCAAAATT | | AUGAUACCCAAAAUU |
| STMN2_ | − | CTTT | 1337 | TGATGATACCCAAAA | 3343 | UGAUGAUACCCAAAA |
| intron1 | | | | TTAGTTTATACTTAT | | UUAGUUUAUACUUAU |
| STMN2_ | − | TTTT | 1338 | TGGTACACCTCCTCA | 3344 | UGGUACACCUCCUCA |
| intron1 | | | | GTATCACATACCTGC | | GUAUCACAUACCUGC |
| STMN2_ | − | GTTT | 1339 | TTGGTACACCTCCTC | 3345 | UUGGUACACCUCCUC |
| intron1 | | | | AGTATCACATACCTG | | AGUAUCACAUACCUG |
| STMN2_ | − | CTTA | 1340 | GAAGATGGGAAAAAT | 3346 | GAAGAUGGGAAAAAU |
| intron1 | | | | AACAGCAGTCAGTTT | | AACAGCAGUCAGUUU |
| STMN2_ | − | TTTA | 1341 | AATGGAAAAGAAAGA | 3347 | AAUGGAAAAGAAAGA |
| intron1 | | | | CAGACTTAGAAGATG | | CAGACUUAGAAGAUG |
| STMN2_ | | CTTT | 1342 | AAATGGAAAAGAAAG | 3348 | AAAUGGAAAAGAAAG |
| intron1 | | | | ACAGACTTAGAAGAT | | ACAGACUUAGAAGAU |
| STMN2_ | − | TTTA | 1343 | AAAAGGTATCTTTAA | 3349 | AAAAGGUAUCUUUAA |
| intron1 | | | | ATGGAAAAGAAAGAC | | AUGGAAAAGAAAGAC |
| STMN2_ | − | ATTT | 1344 | AAAAAGGTATCTTTA | 3350 | AAAAAGGUAUCUUUA |
| intron1 | | | | AATGGAAAAGAAAGA | | AAUGGAAAAGAAAGA |
| STMN2_ | | ATTA | 1345 | GATTTAAAAAGGTAT | 3351 | GAUUUAAAAAGGUAU |
| intron1 | | | | CTTTAAATGGAAAAG | | CUUUAAAUGGAAAAG |
| STMN2_ | − | ATTG | 1346 | GATTAGATTTAAAAA | 3352 | GAUUAGAUUUAAAAA |
| intron1 | | | | GGTATCTTTAAATGG | | GGUAUCUUUAAAUGG |
| STMN2_ | − | ATTG | 1347 | AAATCACATTGGATT | 3353 | AAAUCACAUUGGAUU |
| intron1 | | | | AGATTTAAAAAGGTA | | AGAUUUAAAAAGGUA |
| STMN2_ | − | GTTG | 1348 | AAATCTGATAAAACT | 3354 | AAAUCUGAUAAAACU |
| intron1 | | | | AGATTGAAATCACAT | | AGAUUGAAAUCACAU |
| STMN2_ | − | ATTG | 1349 | TTGAAATCTGATAAA | 3355 | UUGAAAUCUGAUAAA |
| intron1 | | | | ACTAGATTGAAATCA | | ACUAGAUUGAAAUCA |
| STMN2_ | − | TTTC | 1350 | TAATAAACAGAAAAC | 3356 | UAAUAAACAGAAAAC |
| intron1 | | | | CACTACAAGGAGATG | | CACUACAAGGAGAUG |
| STMN2_ | − | GTTA | 1351 | ATGCAGACACCGAGG | 3357 | AUGCAGACACCGAGG |
| intron1 | | | | TTTTCCAATGGACAG | | UUUUCCAAUGGACAG |
| STMN2_ | | TTTT | 1352 | CTAATAAACAGAAAA | 3358 | CUAAUAAACAGAAAA |
| intron1 | | | | CCACTACAAGGAGAT | | CCACUACAAGGAGAU |
| STMN2_ | − | ATTA | 1353 | ACATCGATTTTCTAA | 3359 | ACAUCGAUUUUCUAA |
| intron1 | | | | TAAACAGAAAACCAC | | UAAACAGAAAACCAC |
| STMN2_ | − | GTTA | 1354 | AAATTAACATCGATT | 3360 | AAAUUAACAUCGAUU |
| intron1 | | | | TTCTAATAAACAGAA | | UUCUAAUAAACAGAA |
| STMN2_ | − | CTTC | 1355 | GTTAAAATTAACATC | 3361 | GUUAAAAUUAACAUC |
| intron1 | | | | GATTTTCTAATAAAC | | GAUUUUCUAAUAAAC |
| STMN2_ | − | CTTA | 1356 | CTTCGTTAAAATTAA | 3362 | CUUCGUUAAAAUUAA |
| intron1 | | | | CATCGATTTTCTAAT | | CAUCGAUUUUCUAAU |
| STMN2_ | − | CTTC | 1357 | TTACTTCGTTAAAAT | 3363 | UUACUUCGUUAAAAU |
| intron1 | | | | TAACATCGATTTTCT | | UAACAUCGAUUUUCU |
| STMN2_ | | TTTC | 1358 | TTCTTACTTCGTTAA | 3364 | UUCUUACUUCGUUAA |
| intron1 | | | | AATTAACATCGATTT | | AAUUAACAUCGAUUU |
| STMN2_ | − | ATTT | 1359 | CTTCTTACTTCGTTA | 3365 | CUUCUUACUUCGUUA |
| intron1 | | | | AAATTAACATCGATT | | AAAUUAACAUCGAUU |
| STMN2_ | − | CTTA | 1360 | TATATTTCTTCTTAC | 3366 | UAUAUUUCUUCUUAC |
| intron1 | | | | TTCGTTAAAATTAAC | | UUCGUUAAAAUUAAC |
| STMN2_ | − | TTTA | 1361 | TACTTATATATTTCT | 3367 | UACUUAUAUAUUUCU |
| intron1 | | | | TCTTACTTCGTTAAA | | UCUUACUUCGUUAAA |
| STMN2_ | − | GTTT | 1362 | ATACTTATATATTTC | 3368 | AUACUUAUAUAUUUC |
| intron1 | | | | TTCTTACTTCGTTAA | | UUCUUACUUCGUUAA |
| STMN2_ | − | ATTA | 1363 | GTTTATACTTATATA | 3369 | GUUUAUACUUAUAUA |
| intron1 | | | | TTTCTTCTTACTTCG | | UUUCUUCUUACUUCG |
| STMN2_ | − | TTTG | 1364 | ATGATACCCAAAATT | 3370 | AUGAUACCCAAAAUU |
| intron1 | | | | AGTTTATACTTATAT | | AGUUUAUACUUAUAU |
| STMN2_ | − | TTTT | 1365 | GATGATACCCAAAAT | 3371 | GAUGAUACCCAAAAU |
| intron1 | | | | TAGTTTATACTTATA | | UAGUUUAUACUUAUA |
| STMN2_ | − | ATTT | 1366 | TCTAATAAACAGAAA | 3372 | UCUAAUAAACAGAAA |
| intron1 | | | | ACCACTACAAGGAGA | | ACCACUACAAGGAGA |
| STMN2_ | − | GTTT | 1367 | TCCAATGGACAGAAC | 3373 | UCCAAUGGACAGAAC |
| intron1 | | | | CAGTCTAGGTTCTGA | | CAGUCUAGGUUCUGA |
| STMN2_ | − | TTTT | 1368 | CCAATGGACAGAACC | 3374 | CCAAUGGACAGAACC |
| intron1 | | | | AGTCTAGGTTCTGAA | | AGUCUAGGUUCUGAA |
| STMN2_ | − | TTTC | 1369 | CAATGGACAGAACCA | 3375 | CAAUGGACAGAACCA |
| intron1 | | | | GTCTAGGTTCTGAAA | | GUCUAGGUUCUGAAA |
| STMN2_ | − | TTTT | 1370 | AGAATAGAATAATTT | 3376 | AGAAUAGAAUAAUUU |
| intron1 | | | | ACTACAAATCTGTAA | | ACUACAAAUCUGUAA |
| STMN2_ | − | CTTT | 1371 | TAGAATAGAATAATT | 3377 | UAGAAUAGAAUAAUU |
| intron1 | | | | TACTACAAATCTGTA | | UACUACAAAUCUGUA |
| STMN2_ | − | TTTC | 1372 | TCTTTTAGAATAGAA | 3378 | UCUUUUAGAAUAGAA |
| intron1 | | | | TAATTTACTACAAAT | | UAAUUUACUACAAAU |
| STMN2_ | − | ATTT | 1373 | CTCTTTTAGAATAGA | 3379 | CUCUUUUAGAAUAGA |
| intron1 | | | | ATAATTTACTACAAA | | AUAAUUUACUACAAA |
| STMN2_ | − | ATTA | 1374 | ATGAGGTAATAGCTG | 3380 | AUGAGGUAAUAGCUG |
| intron1 | | | | TAACAATAAAAACAC | | UAACAAUAAAAACAC |
| STMN2_ | − | TTTG | 1375 | CTAAAAATATTAATG | 3381 | CUAAAAAUAUUAAUG |
| intron1 | | | | AGGTAATAGCTGTAA | | AGGUAAUAGCUGUAA |
| STMN2_ | − | GTTT | 1376 | GCTAAAAATATTAAT | 3382 | GCUAAAAAUAUUAAU |
| intron1 | | | | GAGGTAATAGCTGTA | | GAGGUAAUAGCUGUA |
| STMN2_ | − | TTTC | 1377 | AATGCAACAAATAAA | 3383 | AAUGCAACAAAUAAA |
| intron1 | | | | AGTTTGCTAAAAATA | | AGUUUGCUAAAAAUA |
| STMN2_ | − | CTTT | 1378 | CAATGCAACAAATAA | 3384 | CAAUGCAACAAAUAA |
| intron1 | | | | AAGTTTGCTAAAAAT | | AAGUUUGCUAAAAAU |
| STMN2_ | − | ATTA | 1379 | AAACTGCTTTCAATG | 3385 | AAACUGCUUUCAAUG |
| intron1 | | | | CAACAAATAAAAGTT | | CAACAAAUAAAAGUU |
| STMN2_ | − | TTTG | 1380 | AAAAATAAAAACCCA | 3386 | AAAAAUAAAAACCCA |
| intron1 | | | | AAGTAATTAAAACTG | | AAGUAAUUAAAACUG |
| STMN2_ | − | ATTT | 1381 | GAAAAATAAAAACCC | 3387 | GAAAAAUAAAAACCC |
| intron1 | | | | AAAGTAATTAAAACT | | AAAGUAAUUAAAACU |
| STMN2_ | − | ATTA | 1382 | GTAATTTGAAAAATA | 3388 | GUAAUUUGAAAAAUA |
| intron1 | | | | AAAACCCAAAGTAAT | | AAAACCCAAAGUAAU |
| STMN2_ | − | ATTC | 1383 | CACCATCTATCCATT | 3389 | CACCAUCUAUCCAUU |
| intron1 | | | | AGTAATTTGAAAAAT | | AGUAAUUUGAAAAAU |
| STMN2_ | − | CTTA | 1384 | TTCCACCATCTATCC | 3390 | UUCCACCAUCUAUCC |
| intron1 | | | | ATTAGTAATTTGAAA | | AUUAGUAAUUUGAAA |
| STMN2_ | − | ATTA | 1385 | AATGCTTATTCCACC | 3391 | AAUGCUUAUUCCACC |
| intron1 | | | | ATCTATCCATTAGTA | | AUCUAUCCAUUAGUA |
| STMN2_ | − | ATTG | 1386 | TGCCAAATGATTAAA | 3392 | UGCCAAAUGAUUAAA |
| intron1 | | | | TGCTTATTCCACCAT | | UGCUUAUUCCACCAU |
| STMN2_ | − | TTTG | 1387 | ATGGAAGTCATATTG | 3393 | AUGGAAGUCAUAUUG |
| intron1 | | | | TGCCAAATGATTAAA | | UGCCAAAUGAUUAAA |
| STMN2_ | − | ATTT | 1388 | GATGGAAGTCATATT | 339 | GAUGGAAGUCAUAUU |
| intron1 | | | | GTGCCAAATGATTAA | 4 | GUGCCAAAUGAUUAA |
| STMN2_ | − | TTTA | 1389 | ATCACTGAGAATGAG | 3395 | AUCACUGAGAAUGAG |
| intron1 | | | | CTATTTGATGGAAGT | | CUAUUUGAUGGAAGU |
| STMN2_ | − | TTTT | 1390 | AATCACTGAGAATGA | 3396 | AAUCACUGAGAAUGA |
| intron1 | | | | GCTATTTGATGGAAG | | GCUAUUUGAUGGAAG |
| STMN2_ | − | TTTT | 1391 | TAATCACTGAGAATG | 3397 | UAAUCACUGAGAAUG |
| intron1 | | | | AGCTATTTGATGGAA | | AGCUAUUUGAUGGAA |
| STMN2_ | − | TTTT | 1392 | TTAATCACTGAGAAT | 3398 | UUAAUCACUGAGAAU |
| intron1 | | | | GAGCTATTTGATGGA | | GAGCUAUUUGAUGGA |
| STMN2_ | − | ATTT | 1393 | TTTAATCACTGAGAA | 3399 | UUUAAUCACUGAGAA |
| intron1 | | | | TGAGCTATTTGATGG | | UGAGCUAUUUGAUGG |
| STMN2_ | − | CTTG | 1394 | TAGCATTTTTTAATC | 3400 | UAGCAUUUUUUAAUC |
| intron1 | | | | ACTGAGAATGAGCTA | | ACUGAGAAUGAGCUA |
| STMN2_ | − | ATTG | 1395 | TAGCCTCTTGTAGCA | 3401 | UAGCCUCUUGUAGCA |
| intron1 | | | | TTTTTTAATCACTGA | | UUUUUUAAUCACUGA |
| STMN2_ | − | TTTC | 1396 | CTGAATCTGAGTAAA | 3402 | CUGAAUCUGAGUAAA |
| intron1 | | | | TTGTAGCCTCTTGTA | | UUGUAGCCUCUUGUA |
| STMN2_ | − | TTTA | 1397 | GAATAGAATAATTTA | 3403 | GAAUAGAAUAAUUUA |
| intron1 | | | | CTACAAATCTGTAAG | | CUACAAAUCUGUAAG |
| STMN2_ | − | ATTT | 1398 | ACTACAAATCTGTAA | 3404 | ACUACAAAUCUGUAA |
| intron1 | | | | GTCACATTATTGTAA | | GUCACAUUAUUGUAA |
| STMN2_ | − | TTTA | 1399 | CTACAAATCTGTAAG | 3405 | CUACAAAUCUGUAAG |
| intron1 | | | | TCACATTATTGTAAA | | UCACAUUAUUGUAAA |
| STMN2_ | − | ATTA | 1400 | TTGTAAAAAAAAACC | 3406 | UUGUAAAAAAAAACC |
| intron1 | | | | ATTGTGAATTTTGAC | | AUUGUGAAUUUUGAC |
| STMN2_ | − | CTTA | 1401 | CTCACCTGGTATAAA | 3407 | CUCACCUGGUAUAAA |
| intron1 | | | | CTAAATACATGAGAT | | CUAAAUACAUGAGAU |
| STMN2_ | − | ATTG | 1402 | CAGGCTCAGCTTACT | 3408 | CAGGCUCAGCUUACU |
| intron1 | | | | CACCTGGTATAAACT | | CACCUGGUAUAAACU |
| STMN2_ | − | TTTA | 1403 | TTGCAGGCTCAGCTT | 340 | UUGCAGGCUCAGCUU |
| intron1 | | | | ACTCACCTGGTATAA | 9 | ACUCACCUGGUAUAA |
| STMN2_ | − | GTTT | 1404 | ATTGCAGGCTCAGCT | 3410 | AUUGCAGGCUCAGCU |
| intron1 | | | | TACTCACCTGGTATA | | UACUCACCUGGUAUA |
| STMN2_ | − | GTTA | 1405 | CACTGGGACAGAGAG | 3411 | CACUGGGACAGAGAG |
| intron1 | | | | TGTTTATTGCAGGCT | | UGUUUAUUGCAGGCU |
| STMN2_ | − | ATTC | 1406 | TAGCTACCTGCGACG | 3412 | UAGCUACCUGCGACG |
| intron1 | | | | TGTTACACTGGGACA | | UGUUACACUGGGACA |
| STMN2_ | − | TTTA | 1407 | TCCTATCATTCTAGC | 3413 | UCCUAUCAUUCUAGC |
| intron1 | | | | TACCTGCGACGTGTT | | UACCUGCGACGUGUU |
| STMN2_ | − | ATTT | 1408 | ATCCTATCATTCTAG | 3414 | AUCCUAUCAUUCUAG |
| intron1 | | | | CTACCTGCGACGTGT | | CUACCUGCGACGUGU |
| STMN2_ | − | ATTA | 1409 | ATTTATCCTATCATT | 3415 | AUUUAUCCUAUCAUU |
| intron1 | | | | CTAGCTACCTGCGAC | | CUAGCUACCUGCGAC |
| STMN2_ | − | TTTA | 1410 | ACGTGCATAGACAAA | 3416 | ACGUGCAUAGACAAA |
| intron1 | | | | CACCACAAGGTCTAT | | CACCACAAGGUCUAU |
| STMN2_ | − | TTTT | 1411 | AACGTGCATAGACAA | 3417 | AACGUGCAUAGACAA |
| intron1 | | | | ACACCACAAGGTCTA | | ACACCACAAGGUCUA |
| STMN2_ | − | ATTT | 1412 | TAACGTGCATAGACA | 3418 | UAACGUGCAUAGACA |
| intron1 | | | | AACACCACAAGGTCT | | AACACCACAAGGUCU |
| STMN2_ | − | TTTC | 1413 | TCTCAGAGAATTTTA | 3419 | UCUCAGAGAAUUUUA |
| intron1 | | | | ACGTGCATAGACAAA | | ACGUGCAUAGACAAA |
| STMN2_ | − | ATTT | 1414 | CCTGAATCTGAGTAA | 3420 | CCUGAAUCUGAGUAA |
| intron1 | | | | ATTGTAGCCTCTTGT | | AUUGUAGCCUCUUGU |
| STMN2_ | − | CTTT | 1415 | CTCTCAGAGAATTTT | 3421 | CUCUCAGAGAAUUUU |
| intron1 | | | | AACGTGCATAGACAA | | AACGUGCAUAGACAA |
| STMN2_ | − | TTTT | 1416 | AAAATATACTTTCTC | 3422 | AAAAUAUACUUUCUC |
| intron1 | | | | TCAGAGAATTTTAAC | | UCAGAGAAUUUUAAC |
| STMN2_ | − | ATTT | 1417 | TAAAATATACTTTCT | 3423 | UAAAAUAUACUUUCU |
| intron1 | | | | CTCAGAGAATTTTAA | | CUCAGAGAAUUUUAA |
| STMN2_ | − | ATTA | 1418 | TCATTTTAAAATATA | 3424 | UCAUUUUAAAAUAUA |
| intron1 | | | | CTTTCTCTCAGAGAA | | CUUUCUCUCAGAGAA |
| STMN2_ | − | CTTA | 1419 | ATTATCATTTTAAAA | 3425 | AUUAUCAUUUUAAAA |
| intron1 | | | | TATACTTTCTCTCAG | | UAUACUUUCUCUCAG |
| STMN2_ | − | TTTA | 1420 | ATAGCACAAATGTCC | 3426 | AUAGCACAAAUGUCC |
| intron1 | | | | AATCTTAATTATCAT | | AAUCUUAAUUAUCAU |
| STMN2_ | − | TTTT | 1421 | AATAGCACAAATGTC | 3427 | AAUAGCACAAAUGUC |
| intron1 | | | | CAATCTTAATTATCA | | CAAUCUUAAUUAUCA |
| STMN2_ | − | ATTT | 1422 | TAATAGCACAAATGT | 3428 | UAAUAGCACAAAUGU |
| intron1 | | | | CCAATCTTAATTATC | | CCAAUCUUAAUUAUC |
| STMN2_ | − | GTTG | 1423 | TAGATTTTAATAGCA | 3429 | UAGAUUUUAAUAGCA |
| intron1 | | | | CAAATGTCCAATCTT | | CAAAUGUCCAAUCUU |
| STMN2_ | − | TTTG | 1424 | ACTAAAGTTGTAGAT | 3430 | ACUAAAGUUGUAGAU |
| intron1 | | | | TTTAATAGCACAAAT | | UUUAAUAGCACAAAU |
| STMN2_ | − | TTTT | 1425 | GACTAAAGTTGTAGA | 3431 | GACUAAAGUUGUAGA |
| intron1 | | | | TTTTAATAGCACAAA | | UUUUAAUAGCACAAA |
| STMN2_ | − | ATTT | 1426 | TGACTAAAGTTGTAG | 3432 | UGACUAAAGUUGUAG |
| intron1 | | | | ATTTTAATAGCACAA | | AUUUUAAUAGCACAA |
| STMN2_ | − | ATTG | 1427 | TGAATTTTGACTAAA | 3433 | UGAAUUUUGACUAAA |
| intron1 | | | | GTTGTAGATTTTAAT | | GUUGUAGAUUUUAAU |
| STMN2_ | − | ATTG | 1428 | TAAAAAAAAACCATT | 3434 | UAAAAAAAAACCAUU |
| intron1 | | | | GTGAATTTTGACTAA | | GUGAAUUUUGACUAA |
| STMN2_ | − | TTTA | 1429 | AAATATACTTTCTCT | 3435 | AAAUAUACUUUCUCU |
| intron1 | | | | CAGAGAATTTTAACG | | CAGAGAAUUUUAACG |
| STMN2_ | − | ATTG | 1430 | ATAAACTACTGCCAT | 3436 | AUAAACUACUGCCAU |
| intron1 | | | | TTCTTTCAGTTTTAT | | UUCUUUCAGUUUUAU |
| STMN2_ | − | CTTA | 1431 | TGGCACTCTGAAAGG | 3437 | UGGCACUCUGAAAGG |
| intron1 | | | | ACATTTCCTGAATCT | | ACAUUUCCUGAAUCU |
| STMN2_ | − | TTTA | 1432 | TTATATGAATCAGCC | 3438 | UUAUAUGAAUCAGCC |
| intron1 | | | | TTATGGCACTCTGAA | | UUAUGGCACUCUGAA |
| STMN2_ | − | CTTT | 1433 | TTTCAGCCTCCTGTG | 3439 | UUUCAGCCUCCUGUG |
| intron1 | | | | AGCAATGAGCTACCA | | AGCAAUGAGCUACCA |
| STMN2_ | − | CTTC | 1434 | CTCCTGCTCGGAGGC | 3440 | CUCCUGCUCGGAGGC |
| intron1 | | | | CAGCTTTTTTCAGCC | | CAGCUUUUUUCAGCC |
| STMN2_ | − | TTTG | 1435 | TGCTCTGAGCTTCCT | 3441 | UGCUCUGAGCUUCCU |
| intron1 | | | | CCTGCTCGGAGGCCA | | CCUGCUCGGAGGCCA |
| STMN2_ | − | GTTT | 1436 | GTGCTCTGAGCTTCC | 3442 | GUGCUCUGAGCUUCC |
| intron1 | | | | TCCTGCTCGGAGGCC | | UCCUGCUCGGAGGCC |
| STMN2_ | − | GTTC | 1437 | GCTATCAGCAGCTCC | 3443 | GCUAUCAGCAGCUCC |
| intron1 | | | | CAGTGGCCACGCCCA | | CAGUGGCCACGCCCA |
| STMN2_ | − | CTTC | 1438 | CCACGACCAAAAAAG | 3444 | CCACGACCAAAAAAG |
| intron1 | | | | AAACTGGTGTGAGCT | | AAACUGGUGUGAGCU |
| STMN2_ | − | TTTC | 1439 | TTCCCACGACCAAAA | 3445 | UUCCCACGACCAAAA |
| intron1 | | | | AAGAAACTGGTGTGA | | AAGAAACUGGUGUGA |
| STMN2_ | − | TTTT | 1440 | CTTCCCACGACCAAA | 3446 | CUUCCCACGACCAAA |
| intron1 | | | | AAAGAAACTGGTGTG | | AAAGAAACUGGUGUG |
| STMN2_ | − | TTTT | 1441 | TCTTCCCACGACCAA | 3447 | UCUUCCCACGACCAA |
| intron1 | | | | AAAAGAAACTGGTGT | | AAAAGAAACUGGUGU |
| STMN2_ | − | GTTT | 1442 | TTCTTCCCACGACCA | 3448 | UUCUUCCCACGACCA |
| intron1 | | | | AAAAAGAAACTGGTG | | AAAAAGAAACUGGUG |
| STMN2_ | − | CTTG | 1443 | TGACAACAGGATAAT | 3449 | UGACAACAGGAUAAU |
| intron1 | | | | ATGTGTTTTTCTTCC | | AUGUGUUUUUCUUCC |
| STMN2_ | − | TTTC | 1444 | ATATAAGGTCACAGA | 3450 | AUAUAAGGUCACAGA |
| intron1 | | | | TCTTGTGACAACAGG | | UCUUGUGACAACAGG |
| STMN2_ | − | TTTT | 1445 | CATATAAGGTCACAG | 3451 | CAUAUAAGGUCACAG |
| intron1 | | | | ATCTTGTGACAACAG | | AUCUUGUGACAACAG |
| STMN2_ | − | TTTT | 1446 | TCATATAAGGTCACA | 3452 | UCAUAUAAGGUCACA |
| intron1 | | | | GATCTTGTGACAACA | | GAUCUUGUGACAACA |
| STMN2_ | − | TTTT | 1447 | TTCATATAAGGTCAC | 3453 | UUCAUAUAAGGUCAC |
| intron1 | | | | AGATCTTGTGACAAC | | AGAUCUUGUGACAAC |
| STMN2_ | − | ATTT | 1448 | TTTCATATAAGGTCA | 3454 | UUUCAUAUAAGGUCA |
| intron1 | | | | CAGATCTTGTGACAA | | CAGAUCUUGUGACAA |
| STMN2_ | − | ATTC | 1449 | TAGCATTTTTTCATA | 3455 | UAGCAUUUUUUCAUA |
| intron1 | | | | TAAGGTCACAGATCT | | UAAGGUCACAGAUCU |
| STMN2_ | − | TTTA | 1450 | ATGAAAAAATTCTAG | 3456 | AUGAAAAAAUUCUAG |
| intron1 | | | | CATTTTTTCATATAA | | CAUUUUUUCAUAUAA |
| STMN2_ | − | TTTT | 1451 | AATGAAAAAATTCTA | 3457 | AAUGAAAAAAUUCUA |
| intron1 | | | | GCATTTTTTCATATA | | GCAUUUUUUCAUAUA |
| STMN2_ | − | TTTT | 1452 | TAATGAAAAAATTCT | 3458 | UAAUGAAAAAAUUCU |
| intron1 | | | | AGCATTTTTTCATAT | | AGCAUUUUUUCAUAU |
| STMN2_ | − | TTTT | 1453 | TTAATGAAAAAATTC | 3459 | UUAAUGAAAAAAUUC |
| intron1 | | | | TAGCATTTTTTCATA | | UAGCAUUUUUUCAUA |
| STMN2_ | − | CTTT | 1454 | TTTAATGAAAAAATT | 3460 | UUUAAUGAAAAAAUU |
| intron1 | | | | CTAGCATTTTTTCAT | | CUAGCAUUUUUUCAU |
| STMN2_ | − | TTTC | 1455 | TTTTTTAATGAAAAA | 3461 | UUUUUUAAUGAAAAA |
| intron1 | | | | ATTCTAGCATTTTTT | | AUUCUAGCAUUUUUU |
| STMN2_ | − | TTTT | 1456 | CTTTTTTAATGAAAA | 3462 | CUUUUUUAAUGAAAA |
| intron1 | | | | AATTCTAGCATTTTT | | AAUUCUAGCAUUUUU |
| STMN2_ | − | ATTT | 1457 | TCTTTTTTAATGAAA | 3463 | UCUUUUUUAAUGAAA |
| intron1 | | | | AAATTCTAGCATTTT | | AAAUUCUAGCAUUUU |
| STMN2_ | − | GTTC | 1458 | AGTATTTTCTTTTTT | 3464 | AGUAUUUUCUUUUUU |
| intron1 | | | | AATGAAAAAATTCTA | | AAUGAAAAAAUUCUA |
| STMN2_ | − | GTTC | 1459 | TGAAAACATCTGGGT | 3465 | UGAAAACAUCUGGGU |
| intron1 | | | | CACTGGCTAGTTCAG | | CACUGGCUAGUUCAG |
| STMN2_ | − | TTTT | 1460 | TTCAGCCTCCTGTGA | 3466 | UUCAGCCUCCUGUGA |
| intron1 | | | | GCAATGAGCTACCAA | | GCAAUGAGCUACCAA |
| STMN2_ | − | TTTT | 1461 | TCAGCCTCCTGTGAG | 3467 | UCAGCCUCCUGUGAG |
| intron1 | | | | CAATGAGCTACCAAG | | CAAUGAGCUACCAAG |
| STMN2_ | − | TTTT | 1462 | CAGCCTCCTGTGAGC | 3468 | CAGCCUCCUGUGAGC |
| intron1 | | | | AATGAGCTACCAAGG | | AAUGAGCUACCAAGG |
| STMN2_ | − | TTTC | 1463 | AGCCTCCTGTGAGCA | 3469 | AGCCUCCUGUGAGCA |
| intron1 | | | | ATGAGCTACCAAGGT | | AUGAGCUACCAAGGU |
| STMN2_ | − | TTTT | 1464 | ATTATATGAATCAGC | 3470 | AUUAUAUGAAUCAGC |
| intron1 | | | | CTTATGGCACTCTGA | | CUUAUGGCACUCUGA |
| STMN2_ | − | ATTT | 1465 | TATTATATGAATCAG | 3471 | UAUUAUAUGAAUCAG |
| intron1 | | | | CCTTATGGCACTCTG | | CCUUAUGGCACUCUG |
| STMN2_ | − | ATTA | 1466 | TAGGGAAGAAAACTA | 3472 | UAGGGAAGAAAACUA |
| intron1 | | | | TTTTATTATATGAAT | | UUUUAUUAUAUGAAU |
| STMN2_ | − | CTTA | 1467 | AATTATAGGGAAGAA | 3473 | AAUUAUAGGGAAGAA |
| intron1 | | | | AACTATTTTATTATA | | AACUAUUUUAUUAUA |
| STMN2_ | − | TTTG | 1468 | ATCTTAAATTATAGG | 3474 | AUCUUAAAUUAUAGG |
| intron1 | | | | GAAGAAAACTATTTT | | GAAGAAAACUAUUUU |
| STMN2_ | − | ATTT | 1469 | GATCTTAAATTATAG | 3475 | GAUCUUAAAUUAUAG |
| intron1 | | | | GGAAGAAAACTATTT | | GGAAGAAAACUAUUU |
| STMN2_ | − | ATTC | 1470 | ACAGAACTAAGTAAC | 3476 | ACAGAACUAAGUAAC |
| intron1 | | | | TATTTGATCTTAAAT | | UAUUUGAUCUUAAAU |
| STMN2_ | − | TTTG | 1471 | ATAGCTACTGCTAGG | 3477 | AUAGCUACUGCUAGG |
| intron1 | | | | TATTCACAGAACTAA | | UAUUCACAGAACUAA |
| STMN2_ | − | GTTT | 1472 | GATAGCTACTGCTAG | 3478 | GAUAGCUACUGCUAG |
| intron1 | | | | GTATTCACAGAACTA | | GUAUUCACAGAACUA |
| STMN2_ | − | ATTC | 1473 | TGTTTGATAGCTACT | 3479 | UGUUUGAUAGCUACU |
| intron1 | | | | GCTAGGTATTCACAG | | GCUAGGUAUUCACAG |
| STMN2_ | − | TTTA | 1474 | AAATTCTGTTTGATA | 3480 | AAAUUCUGUUUGAUA |
| intron1 | | | | GCTACTGCTAGGTAT | | GCUACUGCUAGGUAU |
| STMN2_ | − | CTTT | 1475 | AAAATTCTGTTTGAT | 3481 | AAAAUUCUGUUUGAU |
| intron1 | | | | AGCTACTGCTAGGTA | | AGCUACUGCUAGGUA |
| STMN2_ | − | TTTA | 1476 | ACTTTAAAATTCTGT | 3482 | ACUUUAAAAUUCUGU |
| intron1 | | | | TTGATAGCTACTGCT | | UUGAUAGCUACUGCU |
| STMN2_ | − | ATTA | 1477 | TATGAATCAGCCTTAT | 3483 | UAUGAAUCAGCCUUAU |
| intron1 | | | | GGCACTCTGAAAGG | | GGCACUCUGAAAGG |
| STMN2_ | − | ATTT | 1478 | AACTTTAAAATTCTGT | 3484 | AACUUUAAAAUUCUGU |
| intron1 | | | | TTGATAGCTACTGC | | UUGAUAGCUACUGC |
| STMN2_ | − | GTTA | 1479 | GTTGTACAGATTTAAC | 3485 | GUUGUACAGAUUUAAC |
| intron1 | | | | TTTAAAATTCTGTT | | UUUAAAAUUCUGUU |
| STMN2_ | − | ATTG | 1480 | TTAGTTGTACAGATTT | 3486 | UUAGUUGUACAGAUUU |
| intron1 | | | | AACTTTAAAATTCT | | AACUUUAAAAUUCU |
| STMN2_ | − | CTTC | 1481 | ATTGTTAGTTGTACAG | 3487 | AUUGUUAGUUGUACAG |
| intron1 | | | | ATTTAACTTTAAAA | | AUUUAACUUUAAAA |
| STMN2_ | − | ATTC | 1482 | ATCCTCCACTTCATTG | 3488 | AUCCUCCACUUCAUUG |
| intron1 | | | | TTAGTTGTACAGAT | | UUAGUUGUACAGAU |
| STMN2_ | − | ATTC | 1483 | AATATGTATCGATTCA | 3489 | AAUAUGUAUCGAUUCA |
| intron1 | | | | TCCTCCACTTCATT | | UCCUCCACUUCAUU |
| STMN2_ | − | CTTC | 1484 | CATTCAATATGTATCG | 3490 | CAUUCAAUAUGUAUCG |
| intron1 | | | | ATTCATCCTCCACT | | AUUCAUCCUCCACU |
| STMN2_ | − | TTTA | 1485 | TCAATGACAAAGTCTT | 3491 | UCAAUGACAAAGUCUU |
| intron1 | | | | CCATTCAATATGTA | | CCAUUCAAUAUGUA |
| STMN2_ | − | ATTT | 1486 | ATCAATGACAAAGTC | 3492 | AUCAAUGACAAAGUCU |
| intron1 | | | | TTCCATTCAATATGT | | UCCAUUCAAUAUGU |
| STMN2_ | − | TTTC | 1487 | CTAAAGATGGCCTGA | 3493 | CUAAAGAUGGCCUGAA |
| intron1 | | | | ATTTATCAATGACAA | | UUUAUCAAUGACAA |
| STMN2_ | − | TTTT | 1488 | CCTAAAGATGGCCTG | 3494 | CCUAAAGAUGGCCUG |
| intron1 | | | | AATTTATCAATGACA | | AAUUUAUCAAUGACA |
| STMN2_ | − | ATTT | 1489 | TCCTAAAGATGGCCT | 3495 | UCCUAAAGAUGGCCU |
| intron1 | | | | GAATTTATCAATGAC | | GAAUUUAUCAAUGAC |
| STMN2_ | − | ATTG | 1490 | ATAAATCCGGAATTT | 3496 | AUAAAUCCGGAAUUUU |
| intron1 | | | | TCCTAAAGATGGCCT | | CCUAAAGAUGGCCU |
| STMN2_ | − | GTTG | 1491 | AAGTAAAAAATAATG | 3497 | AAGUAAAAAAUAAUGG |
| intron1 | | | | GTGATTGATAAATCC | | UGAUUGAUAAAUCC |
| STMN2_ | − | GTTG | 1492 | TACAGATTTAACTTT | 3498 | UACAGAUUUAACUUU |
| intron1 | | | | AAAATTCTGTTTGAT | | AAAAUUCUGUUUGAU |
| STMN2_ | + | GTTC | 1493 | CTCACCCTTGGTGGA | 3499 | CUCACCCUUGGUGGA |
| intron1 | | | | TTTAGTCTTTTGCAG | | UUUAGUCUUUUGCAG |
| STMN2_ | − | TTTC | 1494 | AATCGATGAAGAAGT | 3500 | AAUCGAUGAAGAAGU |
| intron1 | | | | AAACAATGATTTTCT | | AAACAAUGAUUUUCU |
| STMN2_ | + | GTTC | 1495 | TGAAGCCTGTGCCAG | 3501 | UGAAGCCUGUGCCAG |
| intron1 | | | | GTATTATGAGAACAA | | GUAUUAUGAGAACAA |
| STMN2_ | + | GTTA | 1496 | CTTAGTTCTGTGAAT | 3502 | CUUAGUUCUGUGAAU |
| intron1 | | | | ACCTAGCAGTAGCTA | | ACCUAGCAGUAGCUA |
| STMN2_ | + | TTTA | 1497 | AGATCAAATAGTTACT | 3503 | AGAUCAAAUAGUUACU |
| intron1 | | | | TAGTTCTGTGAATA | | UAGUUCUGUGAAUA |
| STMN2_ | + | ATTT | 1498 | AAGATCAAATAGTTA | 3504 | AAGAUCAAAUAGUUAC |
| intron1 | | | | CTTAGTTCTGTGAAT | | UUAGUUCUGUGAAU |
| STMN2_ | + | CTTC | 1499 | CCTATAATTTAAGATC | 3505 | CCUAUAAUUUAAGAUC |
| intron1 | | | | AAATAGTTACTTAG | | AAAUAGUUACUUAG |
| STMN2_ | + | TTTC | 1500 | TTCCCTATAATTTAA | 3506 | UUCCCUAUAAUUUAAG |
| intron1 | | | | GATCAAATAGTTACT | | AUCAAAUAGUUACU |
| STMN2_ | + | TTTT | 1501 | CTTCCCTATAATTTA | 3507 | CUUCCCUAUAAUUU |
| intron1 | | | | AGATCAAATAGTTAC | | AAGAUCAAAUAGUUAC |
| STMN2_ | + | GTTT | 1502 | TCTTCCCTATAATTT | 3508 | UCUUCCCUAUAAUUUA |
| intron1 | | | | AAGATCAAATAGTTA | | AGAUCAAAUAGUUA |
| STMN2_ | + | ATTC | 1503 | ATATAATAAAATAGTT | 3509 | AUAUAAUAAAAUAGUU |
| intron1 | | | | TTCTTCCCTATAAT | | UUCUUCCCUAUAAU |
| STMN2_ | + | TTTC | 1504 | AGAGTGCCATAAGGC | 3510 | AGAGUGCCAUAAGGCU |
| intron1 | | | | TGATTCATATAATAA | | GAUUCAUAUAAUAA |
| STMN2_ | + | CTTT | 1505 | CAGAGTGCCATAAGG | 3511 | CAGAGUGCCAUAAGGC |
| intron1 | | | | CTGATTCATATAATA | | UGAUUCAUAUAAUA |
| STMN2_ | + | ATTC | 1506 | AGGAAATGTCCTTTC | 3512 | AGGAAAUGUCCUUUCA |
| intron1 | | | | AGAGTGCCATAAGGC | | GAGUGCCAUAAGGC |
| STMN2_ | + | TTTA | 1507 | CTCAGATTCAGGAAA | 3513 | CUCAGAUUCAGGAAAU |
| intron1 | | | | TGTCCTTTCAGAGTG | | GUCCUUUCAGAGUG |
| STMN2_ | + | ATTT | 1508 | ACTCAGATTCAGGAA | 3514 | ACUCAGAUUCAGGAAA |
| intron1 | | | | ATGTCCTTTCAGAGT | | UGUCCUUUCAGAGU |
| STMN2_ | + | ATTA | 1509 | AAAAATGCTACAAGA | 3515 | AAAAAUGCUACAAGAG |
| intron1 | | | | GGCTACAATTTACTC | | GCUACAAUUUACUC |
| STMN2_ | + | ATTC | 1510 | TCAGTGATTAAAAAA | 3516 | UCAGUGAUUAAAAAAU |
| intron1 | | | | TGCTACAAGAGGCTA | | GCUACAAGAGGCUA |
| STMN2_ | + | CTTC | 1511 | CATCAAATAGCTCATT | 3517 | CAUCAAAUAGCUCAUU |
| intron1 | | | | CTCAGTGATTAAAA | | CUCAGUGAUUAAAA |
| STMN2_ | + | TTTG | 1512 | GCACAATATGACTTCC | 3518 | GCACAAUAUGACUUCC |
| intron1 | | | | ATCAAATAGCTCAT | | AUCAAAUAGCUCAU |
| STMN2_ | + | ATTT | 1513 | GGCACAATATGACTT | 3519 | GGCACAAUAUGACUUC |
| intron1 | | | | CCATCAAATAGCTCA | | CAUCAAAUAGCUCA |
| STMN2_ | + | TTTA | 1514 | ATCATTTGGCACAAT | 3520 | AUCAUUUGGCACAAUA |
| intron1 | | | | ATGACTTCCATCAAA | | UGACUUCCAUCAAA |
| STMN2_ | + | ATTT | 1515 | AATCATTTGGCACAA | 3521 | AAUCAUUUGGCACAAU |
| intron1 | | | | TATGACTTCCATCAA | | AUGACUUCCAUCAA |
| STMN2_ | + | ATTA | 1516 | CTAATGGATAGATGG | 3522 | CUAAUGGAUAGAUGGU |
| intron1 | | | | TGGAATAAGCATTTA | | GGAAUAAGCAUUUA |
| STMN2_ | + | TTTC | 1517 | AAATTACTAATGGAT | 3523 | AAAUUACUAAUGGAU |
| intron1 | | | | AGATGGTGGAATAAG | | AGAUGGUGGAAUAAG |
| STMN2_ | + | TTTT | 1518 | CAAATTACTAATGGA | 3524 | CAAAUUACUAAUGGA |
| intron1 | | | | TAGATGGTGGAATAA | | UAGAUGGUGGAAUAA |
| STMN2_ | + | TTTT | 1519 | TCAAATTACTAATGG | 3525 | UCAAAUUACUAAUGG |
| intron1 | | | | ATAGATGGTGGAATA | | AUAGAUGGUGGAAUA |
| STMN2_ | + | ATTT | 1520 | TTCAAATTACTAATG | 3526 | UUCAAAUUACUAAUG |
| intron1 | | | | GATAGATGGTGGAAT | | GAUAGAUGGUGGAAU |
| STMN2_ | + | TTTA | 1521 | TTTTTCAAATTACTA | 3527 | UUUUUCAAAUUACUA |
| intron1 | | | | ATGGATAGATGGTGG | | AUGGAUAGAUGGUGG |
| STMN2_ | + | TTTT | 1522 | ATTTTTCAAATTACT | 3528 | AUUUUUCAAAUUACU |
| intron1 | | | | AATGGATAGATGGTG | | AAUGGAUAGAUGGUG |
| STMN2_ | + | CTTA | 1523 | GTTCTGTGAATACCT | 3529 | GUUCUGUGAAUACCU |
| intron1 | | | | AGCAGTAGCTATCAA | | AGCAGUAGCUAUCAA |
| STMN2_ | + | TTTT | 1524 | TATTTTTCAAATTAC | 3530 | UAUUUUUCAAAUUAC |
| intron1 | | | | TAATGGATAGATGGT | | UAAUGGAUAGAUGGU |
| STMN2_ | + | GTTC | 1525 | TGTGAATACCTAGCA | 3531 | UGUGAAUACCUAGCA |
| intron1 | | | | GTAGCTATCAAACAG | | GUAGCUAUCAAACAG |
| STMN2_ | + | TTTT | 1526 | AAAGTTAAATCTGTA | 3532 | AAAGUUAAAUCUGUA |
| intron1 | | | | CAACTAACAATGAAG | | CAACUAACAAUGAAG |
| STMN2_ | + | TTTT | 1527 | GGTCGTGGGAAGAAA | 3533 | GGUCGUGGGAAGAAA |
| intron1 | | | | AACACATATTATCCT | | AACACAUAUUAUCCU |
| STMN2_ | + | TTTT | 1528 | TGGTCGTGGGAAGAA | 3534 | UGGUCGUGGGAAGAA |
| intron1 | | | | AAACACATATTATCC | | AAACACAUAUUAUCC |
| STMN2_ | + | TTTT | 1529 | TTGGTCGTGGGAAGA | 3535 | UUGGUCGUGGGAAGA |
| intron1 | | | | AAAACACATATTATC | | AAAACACAUAUUAUC |
| STMN2_ | + | CTTT | 1530 | TTTGGTCGTGGGAAG | 3536 | UUUGGUCGUGGGAAG |
| intron1 | | | | AAAAACACATATTAT | | AAAAACACAUAUUAU |
| STMN2_ | + | TTTC | 1531 | TTTTTTGGTCGTGGG | 3537 | UUUUUUGGUCGUGGG |
| intron1 | | | | AAGAAAAACACATAT | | AAGAAAAACACAUAU |
| STMN2_ | + | GTTT | 1532 | CTTTTTTGGTCGTGG | 3538 | CUUUUUUGGUCGUGG |
| intron1 | | | | GAAGAAAAACACATA | | GAAGAAAAACACAUA |
| STMN2_ | + | ATTG | 1533 | CTCACAGGAGGCTGA | 3539 | CUCACAGGAGGCUGA |
| intron1 | | | | AAAAAGCTGGCCTCC | | AAAAAGCUGGCCUCC |
| STMN2_ | + | CTTG | 1534 | GTAGCTCATTGCTCA | 3540 | GUAGCUCAUUGCUCA |
| intron1 | | | | CAGGAGGCTGAAAAA | | CAGGAGGCUGAAAAA |
| STMN2_ | + | CTTC | 1535 | AACTGAGTGTGACTG | 3541 | AACUGAGUGUGACUG |
| intron1 | | | | ATCACATGCTCAGGC | | AUCACAUGCUCAGGC |
| STMN2_ | + | TTTA | 1536 | CTTCAACTGAGTGTG | 3542 | CUUCAACUGAGUGUG |
| intron1 | | | | ACTGATCACATGCTC | | ACUGAUCACAUGCUC |
| STMN2_ | + | TTTT | 1537 | ACTTCAACTGAGTGT | 3543 | ACUUCAACUGAGUGU |
| intron1 | | | | GACTGATCACATGCT | | GACUGAUCACAUGCU |
| STMN2_ | + | TTTT | 1538 | TACTTCAACTGAGTG | 3544 | UACUUCAACUGAGUG |
| intron1 | | | | TGACTGATCACATGC | | UGACUGAUCACAUGC |
| STMN2_ | + | TTTT | 1539 | TTACTTCAACTGAGT | 3545 | UUACUUCAACUGAGU |
| intron1 | | | | GTGACTGATCACATG | | GUGACUGAUCACAUG |
| STMN2_ | + | ATTT | 1540 | TTTACTTCAACTGAGT | 3546 | UUUACUUCAACUGAG |
| intron1 | | | | GTGACTGATCACAT | | UGUGACUGAUCACAU |
| STMN2_ | + | ATTA | 1541 | TTTTTTACTTCAACTG | 3547 | UUUUUUACUUCAACUG |
| intron1 | | | | AGTGTGACTGATCA | | AGUGUGACUGAUCA |
| STMN2_ | + | TTTA | 1542 | TCAATCACCATTATTT | 3548 | UCAAUCACCAUUAUUU |
| intron1 | | | | TTTACTTCAACTGA | | UUUACUUCAACUGA |
| STMN2_ | + | ATTT | 1543 | ATCAATCACCATTATT | 3549 | AUCAAUCACCAUUAUU |
| intron1 | | | | TTTTACTTCAACTG | | UUUUACUUCAACUG |
| STMN2_ | + | ATTC | 1544 | CGGATTTATCAATCAC | 3550 | CGGAUUUAUCAAUCAC |
| intron1 | | | | CATTATTTTTTACT | | CAUUAUUUUUUACU |
| STMN2_ | + | TTTA | 1545 | GGAAAATTCCGGATTT | 3551 | GGAAAAUUCCGGAUUU |
| intron1 | | | | ATCAATCACCATTA | | AUCAAUCACCAUUA |
| STMN2_ | + | CTTT | 1546 | AGGAAAATTCCGGAT | 3552 | AGGAAAAUUCCGGAUU |
| intron1 | | | | TTATCAATCACCATT | | UAUCAAUCACCAUU |
| STMN2_ | + | ATTC | 1547 | AGGCCATCTTTAGGA | 3553 | AGGCCAUCUUUAGGAA |
| intron1 | | | | AAATTCCGGATTTAT | | AAUUCCGGAUUUAU |
| STMN2_ | + | ATTG | 1548 | ATAAATTCAGGCCAT | 3554 | AUAAAUUCAGGCCAUC |
| intron1 | | | | CTTTAGGAAAATTCC | | UUUAGGAAAAUUCC |
| STMN2_ | + | TTTG | 1549 | TCATTGATAAATTCAG | 3555 | UCAUUGAUAAAUUCAG |
| intron1 | | | | GCCATCTTTAGGAA | | GCCAUCUUUAGGAA |
| STMN2_ | + | CTTT | 1550 | GTCATTGATAAATTC | 3556 | GUCAUUGAUAAAUUCA |
| intron1 | | | | AGGCCATCTTTAGGA | | GGCCAUCUUUAGGA |
| STMN2_ | + | ATTG | 1551 | AATGGAAGACTTTGT | 3557 | AAUGGAAGACUUUGUC |
| intron1 | | | | CATTGATAAATTCAG | | AUUGAUAAAUUCAG |
| STMN2_ | + | GTTA | 1552 | AATCTGTACAACTAA | 3558 | AAUCUGUACAACUAAC |
| intron1 | | | | CAATGAAGTGGAGGA | | AAUGAAGUGGAGGA |
| STMN2_ | + | TTTA | 1553 | AAGTTAAATCTGTACA | 3559 | AAGUUAAAUCUGUACA |
| intron1 | | | | ACTAACAATGAAGT | | ACUAACAAUGAAGU |
| STMN2_ | + | ATTT | 1554 | TAAAGTTAAATCTGTA | 3560 | UAAAGUUAAAUCUGUA |
| intron1 | | | | CAACTAACAATGAA | | CAACUAACAAUGAA |
| STMN2_ | + | GTTT | 1555 | TTATTTTTCAAATTAC | 3561 | UUAUUUUUCAAAUUAC |
| intron1 | | | | TAATGGATAGATGG | | UAAUGGAUAGAUGG |
| STMN2_ | + | TTTG | 1556 | GGTTTTTATTTTTCAA | 3562 | GGUUUUUAUUUUUCAA |
| intron1 | | | | ATTACTAATGGATA | | AUUACUAAUGGAUA |
| STMN2_ | + | CTTT | 1557 | GGGTTTTTATTTTTCA | 3563 | GGGUUUUUAUUUUUC |
| intron1 | | | | AATTACTAATGGAT | | AAAUUACUAAUGGAU |
| STMN2_ | + | TTTT | 1558 | TTACAATAATGTGACT | 3564 | UUACAAUAAUGUGAC |
| intron1 | | | | TACAGATTTGTAGT | | UUACAGAUUUGUAGU |
| STMN2_ | + | TTTT | 1559 | TTTACAATAATGTGA | 3565 | UUUACAAUAAUGUGA |
| intron1 | | | | CTTACAGATTTGTAG | | CUUACAGAUUUGUAG |
| STMN2_ | + | TTTT | 1560 | TTTTACAATAATGTG | 3566 | UUUUACAAUAAUGUGA |
| intron1 | | | | ACTTACAGATTTGTA | | CUUACAGAUUUGUA |
| STMN2_ | + | TTTT | 1561 | TTTTTACAATAATGT | 3567 | UUUUUACAAUAAUGU |
| intron1 | | | | GACTTACAGATTTGT | | GACUUACAGAUUUGU |
| STMN2_ | + | GTTT | 1562 | TTTTTTACAATAATGT | 3568 | UUUUUUACAAUAAUG |
| intron1 | | | | GACTTACAGATTTG | | UGACUUACAGAUUUG |
| STMN2_ | + | ATTC | 1563 | ACAATGGTTTTTTTTT | 3569 | ACAAUGGUUUUUUUUU |
| intron1 | | | | ACAATAATGTGACT | | ACAAUAAUGUGACU |
| STMN2_ | + | TTTA | 1564 | GTCAAAATTCACAAT | 3570 | GUCAAAAUUCACAAUG |
| intron1 | | | | GGTTTTTTTTTACAA | | GUUUUUUUUUACAA |
| STMN2_ | + | CTTT | 1565 | AGTCAAAATTCACAA | 3571 | AGUCAAAAUUCACAAU |
| intron1 | | | | TGGTTTTTTTTTACA | | GGUUUUUUUUUACA |
| STMN2_ | + | ATTA | 1566 | AAATCTACAACTTTA | 3572 | AAAUCUACAACUUUAG |
| intron1 | | | | GTCAAAATTCACAAT | | UCAAAAUUCACAAU |
| STMN2_ | + | TTTG | 1567 | TGCTATTAAAATCTAC | 3573 | UGCUAUUAAAAUCUAC |
| intron1 | | | | AACTTTAGTCAAAA | | AACUUUAGUCAAAA |
| STMN2_ | + | ATTT | 1568 | GTGCTATTAAAATCTA | 3574 | GUGCUAUUAAAAUCUA |
| intron1 | | | | CAACTTTAGTCAAA | | CAACUUUAGUCAAA |
| STMN2_ | + | ATTG | 1569 | GACATTTGTGCTATTA | 3575 | GACAUUUGUGCUAUUA |
| intron1 | | | | AAATCTACAACTTT | | AAAUCUACAACUUU |
| STMN2_ | + | ATTA | 1570 | AGATTGGACATTTGTG | 3576 | AGAUUGGACAUUUGUG |
| intron1 | | | | CTATTAAAATCTAC | | CUAUUAAAAUCUAC |
| STMN2_ | + | TTTA | 1571 | AAATGATAATTAAGA | 3577 | AAAUGAUAAUUAAGAU |
| intron1 | | | | TTGGACATTTGTGCT | | UGGACAUUUGUGCU |
| STMN2_ | + | TTTT | 1572 | AAAATGATAATTAAG | 3578 | AAAAUGAUAAUUAAGA |
| intron1 | | | | ATTGGACATTTGTGC | | UUGGACAUUUGUGC |
| STMN2_ | + | ATTT | 1573 | TAAAATGATAATTAA | 3579 | UAAAAUGAUAAUUAAG |
| intron1 | | | | GATTGGACATTTGTG | | AUUGGACAUUUGUG |
| STMN2_ | + | ATTC | 1574 | TCTGAGAGAAAGTAT | 3580 | UCUGAGAGAAAGUAUA |
| intron1 | | | | ATTTTAAAATGATAA | | UUUUAAAAUGAUAA |
| STMN2_ | + | GTTA | 1575 | AAATTCTCTGAGAGA | 3581 | AAAUUCUCUGAGAGAA |
| intron1 | | | | AAGTATATTTTAAAA | | AGUAUAUUUUAAAA |
| STMN2_ | + | TTTG | 1576 | TCTATGCACGTTAAAA | 3582 | UCUAUGCACGUUAAAA |
| intron1 | | | | TTCTCTGAGAGAAA | | UUCUCUGAGAGAAA |
| STMN2_ | + | GTTT | 1577 | GTCTATGCACGTTAAA | 3583 | GUCUAUGCACGUUAAA |
| intron1 | | | | ATTCTCTGAGAGAA | | AUUCUCUGAGAGAA |
| STMN2_ | + | CTTG | 1578 | TGGTGTTTGTCTATGC | 3584 | UGGUGUUUGUCUAUGC |
| intron1 | | | | ACGTTAAAATTCTC | | ACGUUAAAAUUCUC |
| STMN2_ | + | ATTA | 1579 | ATAGACCTTGTGGTGT | 3585 | AUAGACCUUGUGGUGU |
| intron1 | | | | TTGTCTATGCACGT | | UUGUCUAUGCACGU |
| STMN2_ | + | TTTA | 1580 | TACCAGGTGAGTAAG | 3586 | UACCAGGUGAGUAAGC |
| intron1 | | | | CTGAGCCTGCAATAA | | UGAGCCUGCAAUAA |
| STMN2_ | + | GTTT | 1581 | ATACCAGGTGAGTAA | 3587 | AUACCAGGUGAGUAAG |
| intron1 | | | | GCTGAGCCTGCAATA | | CUGAGCCUGCAAUA |
| STMN2_ | + | TTTA | 1582 | GTTTATACCAGGTGA | 3588 | GUUUAUACCAGGUGAG |
| intron1 | | | | GTAAGCTGAGCCTGC | | UAAGCUGAGCCUGC |
| STMN2_ | + | ATTT | 1583 | AGTTTATACCAGGTG | 3589 | AGUUUAUACCAGGUGA |
| intron1 | | | | AGTAAGCTGAGCCTG | | GUAAGCUGAGCCUG |
| STMN2_ | + | ATTA | 1584 | ATCTCATGTATTTAG | 3590 | AUCUCAUGUAUUUAG |
| intron1 | | | | TTTATACCAGGTGAG | | UUUAUACCAGGUGAG |
| STMN2_ | + | TTTT | 1585 | TACAATAATGTGACTT | 3591 | UACAAUAAUGUGACUU |
| intron1 | | | | ACAGATTTGTAGTA | | ACAGAUUUGUAGUA |
| STMN2_ | + | TTTT | 1586 | ACAATAATGTGACTT | 3592 | ACAAUAAUGUGACUUA |
| intron1 | | | | ACAGATTTGTAGTAA | | CAGAUUUGUAGUAA |
| STMN2_ | + | TTTA | 1587 | CAATAATGTGACTTAC | 3593 | CAAUAAUGUGACUUAC |
| intron1 | | | | AGATTTGTAGTAAA | | AGAUUUGUAGUAAA |
| STMN2_ | + | CTTA | 1588 | CAGATTTGTAGTAAAT | 3594 | CAGAUUUGUAGUAAAU |
| intron1 | | | | TATTCTATTCTAAA | | UAUUCUAUUCUAAA |
| STMN2_ | + | ATTA | 1589 | CTTTGGGTTTTTATTT | 3595 | CUUUGGGUUUUUAUUU |
| intron1 | | | | TTCAAATTACTAAT | | UUCAAAUUACUAAU |
| STMN2_ | + | TTTA | 1590 | ATTACTTTGGGTTTTT | 3596 | AUUACUUUGGGUUUUU |
| intron1 | | | | ATTTTTCAAATTAC | | AUUUUUCAAAUUAC |
| STMN2_ | + | TTTT | 1591 | AATTACTTTGGGTTT | 3597 | AAUUACUUUGGGUUUU |
| intron1 | | | | TTATTTTTCAAATTA | | UAUUUUUCAAAUUA |
| STMN2_ | + | GTTT | 1592 | TAATTACTTTGGGTT | 3598 | UAAUUACUUUGGGUUU |
| intron1 | | | | TTTATTTTTCAAATT | | UUAUUUUUCAAAUU |
| STMN2_ | + | ATTG | 1593 | AAAGCAGTTTTAATTA | 3599 | AAAGCAGUUUUAAUUA |
| intron1 | | | | CTTTGGGTTTTTAT | | CUUUGGGUUUUUAU |
| STMN2_ | + | GTTG | 1594 | CATTGAAAGCAGTTTT | 3600 | CAUUGAAAGCAGUUUU |
| intron1 | | | | AATTACTTTGGGTT | | AAUUACUUUGGGUU |
| STMN2_ | + | TTTG | 1595 | TTGCATTGAAAGCAGT | 3601 | UUGCAUUGAAAGCAGU |
| intron1 | | | | TTTAATTACTTTGG | | UUUAAUUACUUUGG |
| STMN2_ | + | ATTT | 1596 | GTTGCATTGAAAGCA | 3602 | GUUGCAUUGAAAGCAG |
| intron1 | | | | GTTTTAATTACTTTG | | UUUUAAUUACUUUG |
| STMN2_ | + | TTTA | 1597 | TTTGTTGCATTGAAAG | 3603 | UUUGUUGCAUUGAAAG |
| intron1 | | | | CAGTTTTAATTACT | | CAGUUUUAAUUACU |
| STMN2_ | + | TTTT | 1598 | ATTTGTTGCATTGAA | 3604 | AUUUGUUGCAUUGAA |
| intron1 | | | | AGCAGTTTTAATTAC | | AGCAGUUUUAAUUAC |
| STMN2_ | + | CTTT | 1599 | TATTTGTTGCATTGA | 3605 | UAUUUGUUGCAUUGA |
| intron1 | | | | AAGCAGTTTTAATTA | | AAGCAGUUUUAAUUA |
| STMN2_ | + | TTTA | 1600 | GCAAACTTTTATTTG | 3606 | GCAAACUUUUAUUUGU |
| intron1 | | | | TTGCATTGAAAGCAG | | UGCAUUGAAAGCAG |
| STMN2_ | + | TTTT | 1601 | AGCAAACTTTTATTT | 3607 | AGCAAACUUUUAUUU |
| intron1 | | | | GTTGCATTGAAAGCA | | GUUGCAUUGAAAGCA |
| STMN2_ | + | TTTG | 1602 | GTCGTGGGAAGAAAA | 3608 | GUCGUGGGAAGAAAA |
| intron1 | | | | ACACATATTATCCTG | | ACACAUAUUAUCCUG |
| STMN2_ | + | TTTT | 1603 | TAGCAAACTTTTATT | 3609 | UAGCAAACUUUUAUU |
| intron1 | | | | TGTTGCATTGAAAGC | | UGUUGCAUUGAAAGC |
| STMN2_ | + | ATTA | 1604 | ATATTTTTAGCAAACT | 3610 | AUAUUUUUAGCAAACU |
| intron1 | | | | TTTATTTGTTGCAT | | UUUAUUUGUUGCAU |
| STMN2_ | + | ATTA | 1605 | CCTCATTAATATTTT | 3611 | CCUCAUUAAUAUUUU |
| intron1 | | | | TAGCAAACTTTTATT | | UAGCAAACUUUUAUU |
| STMN2_ | + | GTTA | 1606 | CAGCTATTACCTCAT | 3612 | CAGCUAUUACCUCAU |
| intron1 | | | | TAATATTTTTAGCAA | | UAAUAUUUUUAGCAA |
| STMN2_ | + | ATTG | 1607 | TTACAGCTATTACCT | 3613 | UUACAGCUAUUACCU |
| intron1 | | | | CATTAATATTTTTAG | | CAUUAAUAUUUUUAG |
| STMN2_ | + | TTTA | 1608 | TTGTTACAGCTATTA | 3614 | UUGUUACAGCUAUUA |
| intron1 | | | | CCTCATTAATATTTT | | CCUCAUUAAUAUUUU |
| STMN2_ | + | TTTT | 1609 | ATTGTTACAGCTATT | 3615 | AUUGUUACAGCUAUU |
| intron1 | | | | ACCTCATTAATATTT | | ACCUCAUUAAUAUUU |
| STMN2_ | + | TTTT | 1610 | TATTGTTACAGCTAT | 3616 | UAUUGUUACAGCUAU |
| intron1 | | | | TACCTCATTAATATT | | UACCUCAUUAAUAUU |
| STMN2_ | + | GTTT | 1611 | TTATTGTTACAGCTA | 3617 | UUAUUGUUACAGCUA |
| intron1 | | | | TTACCTCATTAATAT | | UUACCUCAUUAAUAU |
| STMN2_ | + | ATTC | 1612 | TAAAAGAGAAATGAG | 3618 | UAAAAGAGAAAUGAG |
| intron1 | | | | TGTTTTTATTGTTAC | | UGUUUUUAUUGUUAC |
| STMN2_ | + | ATTC | 1613 | TATTCTAAAAGAGAA | 3619 | UAUUCUAAAAGAGAA |
| intron1 | | | | ATGAGTGTTTTTATT | | AUGAGUGUUUUUAUU |
| STMN2_ | + | ATTA | 1614 | TTCTATTCTAAAAGA | 3620 | UUCUAUUCUAAAAGA |
| intron1 | | | | GAAATGAGTGTTTTT | | GAAAUGAGUGUUUUU |
| STMN2_ | + | TTTG | 1615 | TAGTAAATTATTCTA | 3621 | UAGUAAAUUAUUCUA |
| intron1 | | | | TTCTAAAAGAGAAAT | | UUCUAAAAGAGAAAU |
| STMN2_ | + | ATTT | 1616 | GTAGTAAATTATTCT | 3622 | GUAGUAAAUUAUUCU |
| intron1 | | | | ATTCTAAAAGAGAAA | | AUUCUAAAAGAGAAA |
| STMN2_ | + | ATTT | 1617 | TTAGCAAACTTTTAT | 3623 | UUAGCAAACUUUUAU |
| intron1 | | | | TTGTTGCATTGAAAG | | UUGUUGCAUUGAAAG |
| STMN2_ | + | ATTA | 1618 | TCCTGTTGTCACAAG | 3624 | UCCUGUUGUCACAAG |
| intron1 | | | | ATCTGTGACCTTATA | | AUCUGUGACCUUAUA |
| STMN2_ | + | GTTG | 1619 | TCACAAGATCTGTGA | 3625 | UCACAAGAUCUGUGA |
| intron1 | | | | CCTTATATGAAAAAA | | CCUUAUAUGAAAAAA |
| STMN2_ | + | CTTA | 1620 | TATGAAAAAATGCTA | 3626 | UAUGAAAAAAUGCUA |
| intron1 | | | | GAATTTTTTCATTAA | | GAAUUUUUUCAUUAA |
| STMN2_ | + | TTTT | 1621 | AAATCTAATCCAATG | 3627 | AAAUCUAAUCCAAUG |
| intron1 | | | | TGATTTCAATCTAGT | | UGAUUUCAAUCUAGU |
| STMN2_ | + | TTTT | 1622 | TAAATCTAATCCAAT | 3628 | UAAAUCUAAUCCAAU |
| intron1 | | | | GTGATTTCAATCTAG | | GUGAUUUCAAUCUAG |
| STMN2_ | + | CTTT | 1623 | TTAAATCTAATCCAA | 3629 | UUAAAUCUAAUCCAA |
| intron1 | | | | TGTGATTTCAATCTA | | UGUGAUUUCAAUCUA |
| STMN2_ | + | TTTA | 1624 | AAGATACCTTTTTAA | 3630 | AAGAUACCUUUUUAA |
| intron1 | | | | ATCTAATCCAATGTG | | AUCUAAUCCAAUGUG |
| STMN2_ | + | ATTT | 1625 | AAAGATACCTTTTTA | 3631 | AAAGAUACCUUUUUA |
| intron1 | | | | AATCTAATCCAATGT | | AAUCUAAUCCAAUGU |
| STMN2_ | + | TTTC | 1626 | CATTTAAAGATACCT | 3632 | CAUUUAAAGAUACCU |
| intron1 | | | | TTTTAAATCTAATCC | | UUUUAAAUCUAAUCC |
| STMN2_ | + | TTTT | 1627 | CCATTTAAAGATACC | 3633 | CCAUUUAAAGAUACC |
| intron1 | | | | TTTTTAAATCTAATC | | UUUUUAAAUCUAAUC |
| STMN2_ | + | CTTT | 1628 | TCCATTTAAAGATAC | 3634 | UCCAUUUAAAGAUA |
| intron1 | | | | CTTTTTAAATCTAAT | | CCUUUUUAAAUCUAAU |
| STMN2_ | + | TTTC | 1629 | TTTTCCATTTAAAGA | 3635 | UUUUCCAUUUAAAGAU |
| intron1 | | | | TACCTTTTTAAATCT | | ACCUUUUUAAAUCU |
| STMN2_ | + | CTTT | 1630 | CTTTTCCATTTAAAG | 3636 | CUUUUCCAUUUAAAGA |
| intron1 | | | | ATACCTTTTTAAATC | | UACCUUUUUAAAUC |
| STMN2_ | + | CTTC | 1631 | TAAGTCTGTCTTTCT | 3637 | UAAGUCUGUCUUUCUU |
| intron1 | | | | TTTCCATTTAAAGAT | | UUCCAUUUAAAGAU |
| STMN2_ | + | TTTC | 1632 | CCATCTTCTAAGTCT | 3638 | CCAUCUUCUAAGUCUG |
| intron1 | | | | GTCTTTCTTTTCCAT | | UCUUUCUUUUCCAU |
| STMN2_ | + | TTTT | 1633 | CCCATCTTCTAAGTC | 3639 | CCCAUCUUCUAAGUCU |
| intron1 | | | | TGTCTTTCTTTTCCA | | GUCUUUCUUUUCCA |
| STMN2_ | + | TTTT | 1634 | TCCCATCTTCTAAGT | 3640 | UCCCAUCUUCUAAGUC |
| intron1 | | | | CTGTCTTTCTTTTCC | | UGUCUUUCUUUUCC |
| STMN2_ | + | ATTT | 1635 | TTCCCATCTTCTAAGT | 3641 | UUCCCAUCUUCUAAGU |
| intron1 | | | | CTGTCTTTCTTTTC | | CUGUCUUUCUUUUC |
| STMN2_ | + | GTTA | 1636 | TTTTTCCCATCTTCTA | 3642 | UUUUUCCCAUCUUCUA |
| intron1 | | | | AGTCTGTCTTTCTT | | AGUCUGUCUUUCUU |
| STMN2_ | + | CTTA | 1637 | GCGAGTAAAACAGGC | 3643 | GCGAGUAAAACAGGCA |
| intron1 | | | | AGGTATGTGATACTG | | GGUAUGUGAUACUG |
| STMN2_ | + | GTTC | 1638 | AGAGCACATCTGAAT | 3644 | AGAGCACAUCUGAAUA |
| intron1 | | | | ATCAGAGTCTCCACC | | UCAGAGUCUCCACC |
| STMN2_ | + | ATTG | 1639 | AAATGTGCCCCCTGTT | 3645 | AAAUGUGCCCCCUGUU |
| intron1 | | | | CAGAGCACATCTGA | | CAGAGCACAUCUGA |
| STMN2_ | + | CTTC | 1640 | ATCGATTGAAATGTGC | 3646 | AUCGAUUGAAAUGUGC |
| intron1 | | | | CCCCTGTTCAGAGC | | CCCCUGUUCAGAGC |
| STMN2_ | + | CTTC | 1641 | TTCATCGATTGAAATG | 3647 | UUCAUCGAUUGAAAUG |
| intron1 | | | | TGCCCCCTGTTCAG | | UGCCCCCUGUUCAG |
| STMN2_ | + | TTTA | 1642 | CTTCTTCATCGATTGA | 3648 | CUUCUUCAUCGAUUGA |
| intron1 | | | | AATGTGCCCCCTGT | | AAUGUGCCCCCUGU |
| STMN2_ | + | GTTT | 1643 | ACTTCTTCATCGATTG | 3649 | ACUUCUUCAUCGAUUG |
| intron1 | | | | AAATGTGCCCCCTG | | AAAUGUGCCCCCUG |
| STMN2_ | + | ATTG | 1644 | TTTACTTCTTCATCGA | 3650 | UUUACUUCUUCAUCGA |
| intron1 | | | | TTGAAATGTGCCCC | | UUGAAAUGUGCCCC |
| STMN2_ | + | ATTA | 1645 | AAGAAAATCATTGTTT | 3651 | AAGAAAAUCAUUGUUU |
| intron1 | | | | ACTTCTTCATCGAT | | ACUUCUUCAUCGAU |
| STMN2_ | + | CTTA | 1646 | AAAATAAAGGAATAA | 3652 | AAAAUAAAGGAAUAAA |
| intron1 | | | | ATTAAAGAAAATCAT | | UUAAAGAAAAUCAU |
| STMN2_ | + | TTTA | 1647 | ATGCTTAAAAATAAA | 3653 | AUGCUUAAAAAUAAAG |
| intron1 | | | | GGAATAAATTAAAGA | | GAAUAAAUUAAAGA |
| STMN2_ | + | TTTA | 1648 | AATCTAATCCAATGTG | 3654 | AAUCUAAUCCAAUGUG |
| intron1 | | | | ATTTCAATCTAGTT | | AUUUCAAUCUAGUU |
| STMN2_ | + | ATTT | 1649 | CAATCTAGTTTTATC | 3655 | CAAUCUAGUUUUAUC |
| intron1 | | | | AGATTTCAACAATTA | | AGAUUUCAACAAUUA |
| STMN2_ | + | TTTC | 1650 | AATCTAGTTTTATCA | 3656 | AAUCUAGUUUUAUCA |
| intron1 | | | | GATTTCAACAATTAT | | GAUUUCAACAAUUAU |
| STMN2_ | + | GTTT | 1651 | TATCAGATTTCAACA | 3657 | UAUCAGAUUUCAACA |
| intron1 | | | | ATTATTGAGCATCTC | | AUUAUUGAGCAUCUC |
| STMN2_ | + | ATTT | 1652 | TTTGATTACATTTTA | 3658 | UUUGAUUACAUUUUA |
| intron1 | | | | TGTAATTCTAATCCA | | UGUAAUUCUAAUCCA |
| STMN2_ | + | ATTA | 1653 | TTTTTTGATTACATT | 3659 | UUUUUUGAUUACAUU |
| intron1 | | | | TTATGTAATTCTAAT | | UUAUGUAAUUCUAAU |
| STMN2_ | + | ATTG | 1654 | ATAGAATTATTTTTT | 3660 | AUAGAAUUAUUUUUU |
| intron1 | | | | GATTACATTTTATGT | | GAUUACAUUUUAUGU |
| STMN2_ | + | TTTA | 1655 | AATATGCATTGATAG | 3661 | AAUAUGCAUUGAUAG |
| intron1 | | | | AATTATTTTTTGATT | | AAUUAUUUUUUGAUU |
| STMN2_ | + | TTTT | 1656 | AAATATGCATTGATA | 3662 | AAAUAUGCAUUGAUA |
| intron1 | | | | GAATTATTTTTTGAT | | GAAUUAUUUUUUGAU |
| STMN2_ | + | TTTT | 1657 | TAAATATGCATTGAT | 3663 | UAAAUAUGCAUUGAU |
| intron1 | | | | AGAATTATTTTTTGA | | AGAAUUAUUUUUUGA |
| STMN2_ | + | TTTT | 1658 | TTAAATATGCATTGA | 3664 | UUAAAUAUGCAUUGA |
| intron1 | | | | TAGAATTATTTTTTG | | UAGAAUUAUUUUUUG |
| STMN2_ | + | ATTT | 1659 | TTTAAATATGCATTG | 3665 | UUUAAAUAUGCAUUG |
| intron1 | | | | ATAGAATTATTTTTT | | AUAGAAUUAUUUUUU |
| STMN2_ | + | TTTG | 1660 | GGTATCATCAAAAGT | 3666 | GGUAUCAUCAAAAGU |
| intron1 | | | | GGATTTTTTAAATAT | | GGAUUUUUUAAAUAU |
| STMN2_ | + | TTTT | 1661 | GGGTATCATCAAAAG | 3667 | GGGUAUCAUCAAAAG |
| intron1 | | | | TGGATTTTTTAAATA | | UGGAUUUUUUAAAUA |
| STMN2_ | + | ATTT | 1662 | TGGGTATCATCAAAA | 3668 | UGGGUAUCAUCAAAA |
| intron1 | | | | GTGGATTTTTTAAAT | | GUGGAUUUUUUAAAU |
| STMN2_ | + | TTTA | 1663 | ACGAAGTAAGAAGAA | 3669 | ACGAAGUAAGAAGAA |
| intron1 | | | | ATATATAAGTATAAA | | AUAUAUAAGUAUAAA |
| STMN2_ | + | TTTT | 1664 | AACGAAGTAAGAAGA | 3670 | AACGAAGUAAGAAGA |
| intron1 | | | | AATATATAAGTATAA | | AAUAUAUAAGUAUAA |
| STMN2_ | + | TTTT | 1665 | AATGCTTAAAAATAA | 3671 | AAUGCUUAAAAAUAA |
| intron1 | | | | AGGAATAAATTAAAG | | AGGAAUAAAUUAAAG |
| STMN2_ | + | ATTT | 1666 | TAACGAAGTAAGAAG | 3672 | UAACGAAGUAAGAAG |
| intron1 | | | | AAATATATAAGTATA | | AAAUAUAUAAGUAUA |
| STMN2_ | + | ATTA | 1667 | GAAAATCGATGTTAA | 3673 | GAAAAUCGAUGUUAA |
| intron1 | | | | TTTTAACGAAGTAAG | | UUUUAACGAAGUAAG |
| STMN2_ | + | TTTA | 1668 | TTAGAAAATCGATGT | 3674 | UUAGAAAAUCGAUGU |
| intron1 | | | | TAATTTTAACGAAGT | | UAAUUUUAACGAAGU |
| STMN2_ | + | GTTT | 1669 | ATTAGAAAATCGATG | 3675 | AUUAGAAAAUCGAUG |
| intron1 | | | | TTAATTTTAACGAAG | | UUAAUUUUAACGAAG |
| STMN2_ | + | TTTC | 1670 | TGTTTATTAGAAAAT | 3676 | UGUUUAUUAGAAAAU |
| intron1 | | | | CGATGTTAATTTTAA | | CGAUGUUAAUUUUAA |
| STMN2_ | + | TTTT | 1671 | CTGTTTATTAGAAAA | 3677 | CUGUUUAUUAGAAAA |
| intron1 | | | | TCGATGTTAATTTTA | | UCGAUGUUAAUUUUA |
| STMN2_ | + | GTTT | 1672 | TCTGTTTATTAGAAA | 3678 | UCUGUUUAUUAGAAA |
| intron1 | | | | ATCGATGTTAATTTT | | AUCGAUGUUAAUUUU |
| STMN2_ | + | CTTG | 1673 | TAGTGGTTTTCTGTTT | 3679 | UAGUGGUUUUCUGUUU |
| intron1 | | | | ATTAGAAAATCGAT | | AUUAGAAAAUCGAU |
| STMN2_ | + | ATTG | 1674 | AGCATCTCCTTGTAGT | 3680 | AGCAUCUCCUUGUAGU |
| intron1 | | | | GGTTTTCTGTTTAT | | GGUUUUCUGUUUAU |
| STMN2_ | + | ATTA | 1675 | TTGAGCATCTCCTTGT | 3681 | UUGAGCAUCUCCUUGU |
| intron1 | | | | AGTGGTTTTCTGTT | | AGUGGUUUUCUGUU |
| STMN2_ | + | TTTC | 1676 | AACAATTATTGAGCA | 3682 | AACAAUUAUUGAGCAU |
| intron1 | | | | TCTCCTTGTAGTGGT | | CUCCUUGUAGUGGU |
| STMN2_ | + | ATTT | 1677 | CAACAATTATTGAGC | 3683 | CAACAAUUAUUGAGCA |
| intron1 | | | | ATCTCCTTGTAGTGG | | UCUCCUUGUAGUGG |
| STMN2_ | + | TTTA | 1678 | TCAGATTTCAACAATT | 3684 | UCAGAUUUCAACAAUU |
| intron1 | | | | ATTGAGCATCTCCT | | AUUGAGCAUCUCCU |
| STMN2_ | + | TTTT | 1679 | ATCAGATTTCAACAA | 3685 | AUCAGAUUUCAACAAU |
| intron1 | | | | TTATTGAGCATCTCC | | UAUUGAGCAUCUCC |
| STMN2_ | + | GTTA | 1680 | ATTTTAACGAAGTAA | 3686 | AUUUUAACGAAGUAAG |
| intron1 | | | | GAAGAAATATATAAG | | AAGAAAUAUAUAAG |
| STMN2_ | + | TTTA | 1681 | TCAATTAATCTCATG | 3687 | UCAAUUAAUCUCAUGU |
| intron1 | | | | TATTTAGTTTATACC | | AUUUAGUUUAUACC |
| STMN2_ | + | ATTT | 1682 | TAATGCTTAAAAATA | 3688 | UAAUGCUUAAAAAUAA |
| intron1 | | | | AAGGAATAAATTAAA | | AGGAAUAAAUUAAA |
| STMN2_ | + | ATTT | 1683 | AGCAGCCGAATATTTT | 3689 | AGCAGCCGAAUAUUUU |
| intron1 | | | | AATGCTTAAAAATA | | AAUGCUUAAAAAUA |
| STMN2_ | + | TTTA | 1684 | CCAGGAACATTCAAG | 3690 | CCAGGAACAUUCAAGU |
| intron1 | | | | TGTTTATTCAATAAG | | GUUUAUUCAAUAAG |
| STMN2_ | + | TTTT | 1685 | ACCAGGAACATTCAA | 3691 | ACCAGGAACAUUCAA |
| intron1 | | | | GTGTTTATTCAATAA | | GUGUUUAUUCAAUAA |
| STMN2_ | + | CTTT | 1686 | TACCAGGAACATTCA | 3692 | UACCAGGAACAUUCA |
| intron1 | | | | AGTGTTTATTCAATA | | AGUGUUUAUUCAAUA |
| STMN2_ | + | TTTC | 1687 | TTTTACCAGGAACAT | 3693 | UUUUACCAGGAACAU |
| intron1 | | | | TCAAGTGTTTATTCA | | UCAAGUGUUUAUUCA |
| STMN2_ | + | TTTT | 1688 | CTTTTACCAGGAACA | 3694 | CUUUUACCAGGAACA |
| intron1 | | | | TTCAAGTGTTTATTC | | UUCAAGUGUUUAUUC |
| STMN2_ | + | CTTT | 1689 | TCTTTTACCAGGAAC | 3695 | UCUUUUACCAGGAAC |
| intron1 | | | | ATTCAAGTGTTTATT | | AUUCAAGUGUUUAUU |
| STMN2_ | + | TTTA | 1690 | ATAAAATCTTTTCTT | 3696 | AUAAAAUCUUUUCUU |
| intron1 | | | | TTACCAGGAACATTC | | UUACCAGGAACAUUC |
| STMN2_ | + | GTTT | 1691 | AATAAAATCTTTTCT | 3697 | AAUAAAAUCUUUUCU |
| intron1 | | | | TTTACCAGGAACATT | | UUUACCAGGAACAUU |
| STMN2_ | + | ATTA | 1692 | ACTGTTTAATAAAAT | 3698 | ACUGUUUAAUAAAAU |
| intron1 | | | | CTTTTCTTTTACCAG | | CUUUUCUUUUACCAG |
| STMN2_ | + | GTTC | 1693 | CAAATATTAACTGTT | 3699 | CAAAUAUUAACUGUU |
| intron1 | | | | TAATAAAATCTTTTC | | UAAUAAAAUCUUUUC |
| STMN2_ | + | ATTG | 1694 | ATGTAAACCTAGTTC | 3700 | AUGUAAACCUAGUUC |
| intron1 | | | | CAAATATTAACTGTT | | CAAAUAUUAACUGUU |
| STMN2_ | + | GTTC | 1695 | TCTAAAAAAGCAGAT | 3701 | UCUAAAAAAGCAGAU |
| intron1 | | | | GATTGATGTAAACCT | | GAUUGAUGUAAACCU |
| STMN2_ | + | TTTC | 1696 | ACCACACTAGAGGGC | 3702 | ACCACACUAGAGGGC |
| intron1 | | | | AATCATGTTCTCTAA | | AAUCAUGUUCUCUAA |
| STMN2_ | + | TTTT | 1697 | CACCACACTAGAGGG | 3703 | CACCACACUAGAGGG |
| intron1 | | | | CAATCATGTTCTCTA | | CAAUCAUGUUCUCUA |
| STMN2_ | + | CTTT | 1698 | TCACCACACTAGAGG | 3704 | UCACCACACUAGAGG |
| intron1 | | | | GCAATCATGTTCTCT | | GCAAUCAUGUUCUCU |
| STMN2_ | + | ATTA | 1699 | ACTTTTCACCACACTA | 3705 | ACUUUUCACCACACUA |
| intron1 | | | | GAGGGCAATCATGT | | GAGGGCAAUCAUGU |
| STMN2_ | + | ATTG | 1700 | GAAAACCTCGGTGTCT | 3706 | GAAAACCUCGGUGUCU |
| intron1 | | | | GCATTAACTTTTCA | | GCAUUAACUUUUCA |
| STMN2_ | + | GTTC | 1701 | TGTCCATTGGAAAACC | 3707 | UGUCCAUUGGAAAACC |
| intron1 | | | | TCGGTGTCTGCATT | | UCGGUGUCUGCAUU |
| STMN2_ | + | TTTC | 1702 | AGAACCTAGACTGGT | 3708 | AGAACCUAGACUGGU |
| intron1 | | | | TCTGTCCATTGGAAA | | UCUGUCCAUUGGAAA |
| STMN2_ | + | TTTT | 1703 | CAGAACCTAGACTGG | 3709 | CAGAACCUAGACUGG |
| intron1 | | | | TTCTGTCCATTGGAA | | UUCUGUCCAUUGGAA |
| STMN2_ | + | GTTT | 1704 | TCAGAACCTAGACTG | 3710 | UCAGAACCUAGACUG |
| intron1 | | | | GTTCTGTCCATTGGA | | GUUCUGUCCAUUGGA |
| STMN2_ | + | ATTA | 1705 | AAAAAGAAAATACTG | 3711 | AAAAAGAAAAUACUGA |
| intron1 | | | | AACTAGCCAGTGACC | | ACUAGCCAGUGACC |
| STMN2_ | + | TTTC | 1706 | ATTAAAAAAGAAAAT | 3712 | AUUAAAAAAGAAAAUA |
| intron1 | | | | ACTGAACTAGCCAGT | | CUGAACUAGCCAGU |
| STMN2_ | + | TTTT | 1707 | CATTAAAAAAGAAAA | 3713 | CAUUAAAAAAGAAAA |
| intron1 | | | | TACTGAACTAGCCAG | | UACUGAACUAGCCAG |
| STMN2_ | + | TTTT | 1708 | TCATTAAAAAAGAAA | 3714 | UCAUUAAAAAAGAAA |
| intron1 | | | | ATACTGAACTAGCCA | | AUACUGAACUAGCCA |
| STMN2_ | + | TTTT | 1709 | TTCATTAAAAAAGAA | 3715 | UUCAUUAAAAAAGAAA |
| intron1 | | | | AATACTGAACTAGCC | | AUACUGAACUAGCC |
| STMN2_ | + | ATTT | 1710 | TTTCATTAAAAAAGA | 3716 | UUUCAUUAAAAAAGAA |
| intron1 | | | | AAATACTGAACTAGC | | AAUACUGAACUAGC |
| STMN2_ | + | ATTC | 1711 | AAGTGTTTATTCAATA | 3717 | AAGUGUUUAUUCAAUA |
| intron1 | | | | AGCTGATGCCATGC | | AGCUGAUGCCAUGC |
| STMN2_ | + | GTTT | 1712 | ATTCAATAAGCTGATG | 3718 | AUUCAAUAAGCUGAUG |
| intron1 | | | | CCATGCTTTACCCT | | CCAUGCUUUACCCU |
| STMN2_ | + | TTTA | 1713 | TTCAATAAGCTGATGC | 3719 | UUCAAUAAGCUGAUGC |
| intron1 | | | | CATGCTTTACCCTA | | CAUGCUUUACCCUA |
| STMN2_ | + | ATTC | 1714 | AATAAGCTGATGCCA | 3720 | AAUAAGCUGAUGCCAU |
| intron1 | | | | TGCTTTACCCTAGTG | | GCUUUACCCUAGUG |
| STMN2_ | + | GTTA | 1715 | TTTAGCAGCCGAATA | 3721 | UUUAGCAGCCGAAUAU |
| intron1 | | | | TTTTAATGCTTAAAA | | UUUAAUGCUUAAAA |
| STMN2_ | + | TTTG | 1716 | TAGTGGATAAATAGT | 3722 | UAGUGGAUAAAUAGU |
| intron1 | | | | AGAAAAATGTCAGTA | | AGAAAAAUGUCAGUA |
| STMN2_ | + | TTTT | 1717 | GTAGTGGATAAATAG | 3723 | GUAGUGGAUAAAUAG |
| intron1 | | | | TAGAAAAATGTCAGT | | UAGAAAAAUGUCAGU |
| STMN2_ | + | ATTT | 1718 | TGTAGTGGATAAATA | 3724 | UGUAGUGGAUAAAUA |
| intron1 | | | | GTAGAAAAATGTCAG | | GUAGAAAAAUGUCAG |
| STMN2_ | + | CTTC | 1719 | TGAGATTTTGTAGTG | 3725 | UGAGAUUUUGUAGUG |
| intron1 | | | | GATAAATAGTAGAAA | | GAUAAAUAGUAGAAA |
| STMN2_ | + | GTTA | 1720 | CTTCTGAGATTTTGT | 3726 | CUUCUGAGAUUUUGU |
| intron1 | | | | AGTGGATAAATAGTA | | AGUGGAUAAAUAGUA |
| STMN2_ | + | TTTA | 1721 | TGTTACTTCTGAGAT | 3727 | UGUUACUUCUGAGAU |
| intron1 | | | | TTTGTAGTGGATAAA | | UUUGUAGUGGAUAAA |
| STMN2_ | + | ATTT | 1722 | ATGTTACTTCTGAGA | 3728 | AUGUUACUUCUGAGA |
| intron1 | | | | TTTTGTAGTGGATAA | | UUUUGUAGUGGAUAA |
| STMN2_ | + | ATTA | 1723 | TAATACCATTTATGT | 3729 | UAAUACCAUUUAUGU |
| intron1 | | | | TACTTCTGAGATTTT | | UACUUCUGAGAUUUU |
| STMN2_ | + | GTTA | 1724 | TTATAATACCATTTA | 3730 | UUAUAAUACCAUUUA |
| intron1 | | | | TGTTACTTCTGAGAT | | UGUUACUUCUGAGAU |
| STMN2_ | + | ATTG | 1725 | TTATTATAATACCAT | 3731 | UUAUUAUAAUACCAU |
| intron1 | | | | TTATGTTACTTCTGA | | UUAUGUUACUUCUGA |
| STMN2_ | + | ATTA | 1726 | CATTGTTATTATAAT | 3732 | CAUUGUUAUUAUAAU |
| intron1 | | | | ACCATTTATGTTACT | | ACCAUUUAUGUUACU |
| STMN2_ | + | TTTA | 1727 | TTACATTGTTATTAT | 3733 | UUACAUUGUUAUUAU |
| intron1 | | | | AATACCATTTATGTT | | AAUACCAUUUAUGUU |
| STMN2_ | + | TTTA | 1728 | GCAGCCGAATATTTT | 3734 | GCAGCCGAAUAUUUU |
| intron1 | | | | AATGCTTAAAAATAA | | AAUGCUUAAAAAUAA |
| STMN2_ | + | TTTT | 1729 | ATTACATTGTTATTA | 3735 | AUUACAUUGUUAUUA |
| intron1 | | | | TAATACCATTTATGT | | UAAUACCAUUUAUGU |
| STMN2_ | + | CTTC | 1730 | TCAGTTTTATTACAT | 3736 | UCAGUUUUAUUACAU |
| intron1 | | | | TGTTATTATAATACC | | UGUUAUUAUAAUACC |
| STMN2_ | + | TTTA | 1731 | CTTCTCAGTTTTATT | 3737 | CUUCUCAGUUUUAUU |
| intron1 | | | | ACATTGTTATTATAA | | ACAUUGUUAUUAUAA |
| STMN2_ | + | TTTT | 1732 | ACTTCTCAGTTTTAT | 3738 | ACUUCUCAGUUUUAU |
| intron1 | | | | TACATTGTTATTATA | | UACAUUGUUAUUAUA |
| STMN2_ | + | GTTT | 1733 | TACTTCTCAGTTTTA | 3739 | UACUUCUCAGUUUUA |
| intron1 | | | | TTACATTGTTATTAT | | UUACAUUGUUAUUAU |
| STMN2_ | + | ATTC | 1734 | CCTGATGGTTTTACT | 3740 | CCUGAUGGUUUUACU |
| intron1 | | | | TCTCAGTTTTATTAC | | UCUCAGUUUUAUUAC |
| STMN2_ | + | ATTA | 1735 | TTCCCTGATGGTTTT | 3741 | UUCCCUGAUGGUUUU |
| intron1 | | | | ACTTCTCAGTTTTAT | | ACUUCUCAGUUUUAU |
| STMN2_ | + | GTTA | 1736 | ATTATTCCCTGATGG | 3742 | AUUAUUCCCUGAUGG |
| intron1 | | | | TTTTACTTCTCAGTT | | UUUUACUUCUCAGUU |
| STMN2_ | + | TTTC | 1737 | AAGGAGACAGGATGA | 3743 | AAGGAGACAGGAUGA |
| intron1 | | | | AATGAGTGGTCATAA | | AAUGAGUGGUCAUAA |
| STMN2_ | + | TTTT | 1738 | CAAGGAGACAGGATG | 3744 | CAAGGAGACAGGAUG |
| intron1 | | | | AAATGAGTGGTCATA | | AAAUGAGUGGUCAUA |
| STMN2_ | + | ATTT | 1739 | TCAAGGAGACAGGAT | 3745 | UCAAGGAGACAGGAU |
| intron1 | | | | GAAATGAGTGGTCAT | | GAAAUGAGUGGUCAU |
| STMN2_ | + | CTTG | 1740 | TACAATTTTCAAGGA | 3746 | UACAAUUUUCAAGGA |
| intron1 | | | | GACAGGATGAAATGA | | GACAGGAUGAAAUGA |
| STMN2_ | + | TTTA | 1741 | CCCTAGTGGATGAAC | 3747 | CCCUAGUGGAUGAAC |
| intron1 | | | | AGAGCTTGTACAATT | | AGAGCUUGUACAAUU |
| STMN2_ | + | CTTT | 1742 | ACCCTAGTGGATGAA | 3748 | ACCCUAGUGGAUGAA |
| intron1 | | | | CAGAGCTTGTACAAT | | CAGAGCUUGUACAAU |
| STMN2_ | + | GTTT | 1743 | TATTACATTGTTATT | 3749 | UAUUACAUUGUUAUU |
| intron1 | | | | ATAATACCATTTATG | | AUAAUACCAUUUAUG |
| STMN2_ | + | TTTT | 1744 | TTGATTACATTTTAT | 3750 | UUGAUUACAUUUUAU |
| intron1 | | | | GTAATTCTAATCCAG | | GUAAUUCUAAUCCAG |
| STMN2_ | + | GTTT | 1745 | ATCAATTAATCTCAT | 3751 | AUCAAUUAAUCUCAU |
| intron1 | | | | GTATTTAGTTTATAC | | GUAUUUAGUUUAUAC |
| STMN2_ | + | ATTT | 1746 | CAGGATAAAACTGAA | 3752 | CAGGAUAAAACUGAA |
| intron1 | | | | AGAAATGGCAGTAGT | | AGAAAUGGCAGUAGU |
| STMN2_ | + | GTTT | 1747 | GCGGGAAAAGCTTCT | 3753 | GCGGGAAAAGCUUCU |
| intron1 | | | | AGAACCTAGACATGT | | AGAACCUAGACAUGU |
| STMN2_ | + | TTTG | 1748 | ATCGTTTGCGGGAAA | 3754 | AUCGUUUGCGGGAAA |
| intron1 | | | | AGCTTCTAGAACCTA | | AGCUUCUAGAACCUA |
| STMN2_ | + | CTTT | 1749 | GATCGTTTGCGGGAA | 3755 | GAUCGUUUGCGGGAA |
| intron1 | | | | AAGCTTCTAGAACCT | | AAGCUUCUAGAACCU |
| STMN2_ | + | TTTG | 1750 | AAGACCTTTGATCGT | 3756 | AAGACCUUUGAUCGU |
| intron1 | | | | TTGCGGGAAAAGCTT | | UUGCGGGAAAAGCUU |
| STMN2_ | + | CTTT | 1751 | GAAGACCTTTGATCG | 3757 | GAAGACCUUUGAUCG |
| intron1 | | | | TTTGCGGGAAAAGCT | | UUUGCGGGAAAAGCU |
| STMN2_ | + | GTTC | 1752 | TTTGAAGACCTTTGA | 3758 | UUUGAAGACCUUUGA |
| intron1 | | | | TCGTTTGCGGGAAAA | | UCGUUUGCGGGAAAA |
| STMN2_ | + | TTTA | 1753 | GACATAGACACAGAT | 3759 | GACAUAGACACAGAU |
| intron1 | | | | AAAGGGTTCTTTGAA | | AAAGGGUUCUUUGAA |
| STMN2_ | + | GTTT | 1754 | AGACATAGACACAGA | 3760 | AGACAUAGACACAGA |
| intron1 | | | | TAAAGGGTTCTTTGA | | UAAAGGGUUCUUUGA |
| STMN2_ | + | ATTA | 1755 | CATAGAGTGTTTAGA | 3761 | CAUAGAGUGUUUAGA |
| intron1 | | | | CATAGACACAGATAA | | CAUAGACACAGAUAA |
| STMN2_ | + | TTTC | 1756 | GGAAGCAAATTACAT | 3762 | GGAAGCAAAUUACAU |
| intron1 | | | | AGAGTGTTTAGACAT | | AGAGUGUUUAGACAU |
| STMN2_ | + | TTTT | 1757 | CGGAAGCAAATTACA | 3763 | CGGAAGCAAAUUACA |
| intron1 | | | | TAGAGTGTTTAGACA | | UAGAGUGUUUAGACA |
| STMN2_ | + | CTTT | 1758 | TCGGAAGCAAATTAC | 3764 | UCGGAAGCAAAUUAC |
| intron1 | | | | ATAGAGTGTTTAGAC | | AUAGAGUGUUUAGAC |
| STMN2_ | + | TTTC | 1759 | TTTTCGGAAGCAAAT | 3765 | UUUUCGGAAGCAAAU |
| intron1 | | | | TACATAGAGTGTTTA | | UACAUAGAGUGUUUA |
| STMN2_ | + | TTTT | 1760 | CTTTTCGGAAGCAAA | 3766 | CUUUUCGGAAGCAAA |
| intron1 | | | | TTACATAGAGTGTTT | | UUACAUAGAGUGUUU |
| STMN2_ | + | TTTT | 1761 | TCTTTTCGGAAGCAA | 3767 | UCUUUUCGGAAGCAA |
| intron1 | | | | ATTACATAGAGTGTT | | AUUACAUAGAGUGUU |
| STMN2_ | + | ATTT | 1762 | TTCTTTTCGGAAGCAA | 3768 | UUCUUUUCGGAAGCAA |
| intron1 | | | | ATTACATAGAGTGT | | AUUACAUAGAGUGU |
| STMN2_ | + | GTTA | 1763 | ACATTTTTCTTTTCGG | 3769 | ACAUUUUUCUUUUCG |
| intron1 | | | | AAGCAAATTACATA | | GAAGCAAAUUACAUA |
| STMN2_ | + | TTTA | 1764 | AGAGAGATGGGAAAA | 3770 | AGAGAGAUGGGAAAA |
| intron1 | | | | GTGGGTTAACATTTT | | GUGGGUUAACAUUUU |
| STMN2_ | + | TTTT | 1765 | AAGAGAGATGGGAAA | 3771 | AAGAGAGAUGGGAAAA |
| intron1 | | | | AGTGGGTTAACATTT | | GUGGGUUAACAUUU |
| STMN2_ | + | CTTT | 1766 | TAAGAGAGATGGGAA | 3772 | UAAGAGAGAUGGGAAA |
| intron1 | | | | AAGTGGGTTAACATT | | AGUGGGUUAACAUU |
| STMN2_ | + | GTTC | 1767 | TGCTTTTAAGAGAGAT | 3773 | UGCUUUUAAGAGAGAU |
| intron1 | | | | GGGAAAAGTGGGTT | | GGGAAAAGUGGGUU |
| STMN2_ | + | ATT | 1768 | TTCTGCTTTTAAGAGA | 3774 | UUCUGCUUUUAAGAGA |
| intron1 | | G | | GATGGGAAAAGTGG | | GAUGGGAAAAGUGG |
| STMN2_ | + | CTTC | 1769 | CAAGAGAGACCTGAC | 3775 | CAAGAGAGACCUGACC |
| intron1 | | | | CACTGACCCCGCCCT | | ACUGACCCCGCCCU |
| STMN2_ | + | ATTC | 1770 | GAAAGGGGGTCGGGT | 3776 | GAAAGGGGGUCGGGUG |
| intron1 | | | | GGGGAGCGCAGCGTG | | GGGAGCGCAGCGUG |
| STMN2_ | + | CTTC | 1771 | ATTCGAAAGGGGGTC | 3777 | AUUCGAAAGGGGGUCG |
| intron1 | | | | GGGTGGGGAGCGCAG | | GGUGGGGAGCGCAG |
| STMN2_ | + | TTTG | 1772 | TGTGCGGACCAGCGG | 3778 | UGUGCGGACCAGCGGU |
| intron1 | | | | TCCCGGGGGGAGGCA | | CCCGGGGGGAGGCA |
| STMN2_ | + | CTTT | 1773 | GTGTGCGGACCAGCG | 3779 | GUGUGCGGACCAGCGG |
| intron1 | | | | GTCCCGGGGGGAGGC | | UCCCGGGGGGAGGC |
| STMN2_ | + | TTTG | 1774 | CGGGAAAAGCTTCTA | 3780 | CGGGAAAAGCUUCUAG |
| intron1 | | | | GAACCTAGACATGTG | | AACCUAGACAUGUG |
| STMN2_ | + | TTTC | 1775 | TTTGTGTGCGGACCAG | 3781 | UUUGUGUGCGGACCAG |
| intron1 | | | | CGGTCCCGGGGGGA | | CGGUCCCGGGGGGA |
| STMN2_ | + | CTTC | 1776 | TAGAACCTAGACATG | 3782 | UAGAACCUAGACAUGU |
| intron1 | | | | TGTATGTATAATAAT | | GUAUGUAUAAUAAU |
| STMN2_ | + | GTTA | 1777 | AGCCACGCGAAATTTC | 3783 | AGCCACGCGAAAUUUC |
| intron1 | | | | CGTTTTGTGGGTCA | | CGUUUUGUGGGUCA |
| STMN2_ | + | CTTT | 1778 | TTTTCCCCCAGCCCAA | 3784 | UUUUCCCCCAGCCCAA |
| intron1 | | | | GCCCCCCGCCCACC | | GCCCCCCGCCCACC |
| STMN2_ | + | CTTC | 1779 | TCGCCCACCCACGGT | 3785 | UCGCCCACCCACGGUC |
| intron1 | | | | CCGCGGAGCTCGGGG | | CGCGGAGCUCGGGG |
| STMN2_ | + | ATTC | 1780 | AGGGAGGGCTGTCTC | 3786 | AGGGAGGGCUGUCUCU |
| intron1 | | | | TTCTCGCCCACCCAC | | UCUCGCCCACCCAC |
| STMN2_ | + | CTTC | 1781 | CCAGGGATTCAGGGA | 3787 | CCAGGGAUUCAGGGAG |
| intron1 | | | | GGGCTGTCTCTTCTC | | GGCUGUCUCUUCUC |
| STMN2_ | + | CTTG | 1782 | ATGTGCGCAGACCCC | 3788 | AUGUGCGCAGACCCC |
| intron1 | | | | CGGCGTGGCTCTCAG | | CGGCGUGGCUCUCAG |
| STMN2_ | + | TTTC | 1783 | AGCCCCGCAGTCCAC | 3789 | AGCCCCGCAGUCCAC |
| intron1 | | | | AACGGCCCGAGCACC | | AACGGCCCGAGCACC |
| STMN2_ | + | TTTT | 1784 | CAGCCCCGCAGTCCA | 3790 | CAGCCCCGCAGUCCA |
| intron1 | | | | CAACGGCCCGAGCAC | | CAACGGCCCGAGCAC |
| STMN2_ | + | TTTT | 1785 | TCAGCCCCGCAGTCC | 3791 | UCAGCCCCGCAGUCC |
| intron1 | | | | ACAACGGCCCGAGCA | | ACAACGGCCCGAGCA |
| STMN2_ | + | TTTT | 1786 | TTCAGCCCCGCAGTC | 3792 | UUCAGCCCCGCAGUC |
| intron1 | | | | CACAACGGCCCGAGC | | CACAACGGCCCGAGC |
| STMN2_ | + | CTTT | 1787 | TTTCAGCCCCGCAGT | 3793 | UUUCAGCCCCGCAGU |
| intron1 | | | | CCACAACGGCCCGAG | | CCACAACGGCCCGAG |
| STMN2_ | + | GTTG | 1788 | AGCTGTATGCAGTCC | 3794 | AGCUGUAUGCAGUCC |
| intron1 | | | | TGGAACCTCTTTTTT | | UGGAACCUCUUUUUU |
| STMN2_ | + | GTTG | 1789 | CAGGATGCGGAGACA | 3795 | CAGGAUGCGGAGACA |
| intron1 | | | | GGGAAAGCTGCCGAA | | GGGAAAGCUGCCGAA |
| STMN2_ | + | CTTG | 1790 | GTTGCAGGATGCGGA | 3796 | GUUGCAGGAUGCGGA |
| intron1 | | | | GACAGGGAAAGCTGC | | GACAGGGAAAGCUGC |
| STMN2_ | + | GTTC | 1791 | TGGCGCTCAGTGGCC | 3797 | UGGCGCUCAGUGGCC |
| intron1 | | | | CCGGGGTGAAAAGGC | | CCGGGGUGAAAAGGC |
| STMN2_ | + | CTTG | 1792 | AGTGCCCACGGTTCT | 3798 | AGUGCCCACGGUUCU |
| intron1 | | | | GGCGCTCAGTGGCCC | | GGCGCUCAGUGGCCC |
| STMN2_ | + | CTTG | 1793 | TGCCTTGAGTGCCCA | 3799 | UGCCUUGAGUGCCCA |
| intron1 | | | | CGGTTCTGGCGCTCA | | CGGUUCUGGCGCUCA |
| STMN2_ | + | ATTG | 1794 | GTCTTGTGCCTTGAG | 3800 | GUCUUGUGCCUUGAG |
| intron1 | | | | TGCCCACGGTTCTGG | | UGCCCACGGUUCUGG |
| STMN2_ | + | CTTC | 1795 | ATCCGCAATTGGTCT | 3801 | AUCCGCAAUUGGUCU |
| intron1 | | | | TGTGCCTTGAGTGCC | | UGUGCCUUGAGUGCC |
| STMN2_ | + | ATTC | 1796 | AGGGCCTTCATCCGC | 3802 | AGGGCCUUCAUCCGC |
| intron1 | | | | AATTGGTCTTGTGCC | | AAUUGGUCUUGUGCC |
| STMN2_ | + | ATTC | 1797 | TGGATTCAGGGCCTT | 3803 | UGGAUUCAGGGCCUU |
| intron1 | | | | CATCCGCAATTGGTC | | CAUCCGCAAUUGGUC |
| STMN2_ | + | TTTC | 1798 | ATAAGCTCAGAGAGA | 3804 | AUAAGCUCAGAGAGA |
| intron1 | | | | CAAGACAGTGGAGAC | | CAAGACAGUGGAGAC |
| STMN2_ | + | ATTT | 1799 | CATAAGCTCAGAGAG | 3805 | CAUAAGCUCAGAGAG |
| intron1 | | | | ACAAGACAGTGGAGA | | ACAAGACAGUGGAGA |
| STMN2_ | + | TTTG | 1800 | TGGGTCAGACAGTGC | 3806 | UGGGUCAGACAGUGC |
| intron1 | | | | CAAATATCGGCAATT | | CAAAUAUCGGCAAUU |
| STMN2_ | + | TTTT | 1801 | GTGGGTCAGACAGTG | 3807 | GUGGGUCAGACAGUG |
| intron1 | | | | CCAAATATCGGCAAT | | CCAAAUAUCGGCAAU |
| STMN2_ | + | GTTT | 1802 | TGTGGGTCAGACAGT | 3808 | UGUGGGUCAGACAGU |
| intron1 | | | | GCCAAATATCGGCAA | | GCCAAAUAUCGGCAA |
| STMN2_ | + | TTTC | 1803 | CGTTTTGTGGGTCAG | 3809 | CGUUUUGUGGGUCAG |
| intron1 | | | | ACAGTGCCAAATATC | | ACAGUGCCAAAUAUC |
| STMN2_ | + | ATTT | 1804 | CCGTTTTGTGGGTCA | 3810 | CCGUUUUGUGGGUCA |
| intron1 | | | | GACAGTGCCAAATAT | | GACAGUGCCAAAUAU |
| STMN2_ | + | CTTA | 1805 | AGTTAAGCCACGCGA | 3811 | AGUUAAGCCACGCGA |
| intron1 | | | | AATTTCCGTTTTGTG | | AAUUUCCGUUUUGUG |
| STMN2_ | + | GTTT | 1806 | CTTTGTGTGCGGACC | 3812 | CUUUGUGUGCGGACC |
| intron1 | | | | AGCGGTCCCGGGGGG | | AGCGGUCCCGGGGGG |
| STMN2_ | + | CTTC | 1807 | GAAGGCGCTGGGGTG | 3813 | GAAGGCGCUGGGGUG |
| intron1 | | | | GGGTTTCTTTGTGTG | | GGGUUUCUUUGUGUG |
| STMN2_ | + | TTTA | 1808 | GGGCAAGGGAGGGGA | 3814 | GGGCAAGGGAGGGGA |
| intron1 | | | | AGGAGAGAGGAAGTC | | AGGAGAGAGGAAGUC |
| STMN2_ | + | CTTA | 1809 | AGGGACATTTTGGAA | 3815 | AGGGACAUUUUGGAA |
| intron1 | | | | AGTGCTTTATAACGA | | AGUGCUUUAUAACGA |
| STMN2_ | + | CTTA | 1810 | AATGGGCTTAAGGGA | 3816 | AAUGGGCUUAAGGGA |
| intron1 | | | | CATTTTGGAAAGTGC | | CAUUUUGGAAAGUGC |
| STMN2_ | + | TTTG | 1811 | CCTTAAATGGGCTTA | 3817 | CCUUAAAUGGGCUUA |
| intron1 | | | | AGGGACATTTTGGAA | | AGGGACAUUUUGGAA |
| STMN2_ | + | GTTT | 1812 | GCCTTAAATGGGCTT | 3818 | GCCUUAAAUGGGCUU |
| intron1 | | | | AAGGGACATTTTGGA | | AAGGGACAUUUUGGA |
| STMN2_ | + | CTTA | 1813 | ACTGTTTGCCTTAAA | 3819 | ACUGUUUGCCUUAAA |
| intron1 | | | | TGGGCTTAAGGGACA | | UGGGCUUAAGGGACA |
| STMN2_ | + | ATTA | 1814 | GGACTCAATCGTGAG | 3820 | GGACUCAAUCGUGAG |
| intron1 | | | | GGGAGGAAGCTACCT | | GGGAGGAAGCUACCU |
| STMN2_ | + | TTTA | 1815 | AAATTAGGACTCAAT | 3821 | AAAUUAGGACUCAAU |
| intron1 | | | | CGTGAGGGGAGGAAG | | CGUGAGGGGAGGAAG |
| STMN2_ | + | ATTT | 1816 | AAAATTAGGACTCAA | 3822 | AAAAUUAGGACUCAA |
| intron1 | | | | TCGTGAGGGGAGGAA | | UCGUGAGGGGAGGAA |
| STMN2_ | + | TTTC | 1817 | CATATTTAAAATTAG | 3823 | CAUAUUUAAAAUUAG |
| intron1 | | | | GACTCAATCGTGAGG | | GACUCAAUCGUGAGG |
| STMN2_ | + | GTTT | 1818 | CCATATTTAAAATTA | 3824 | CCAUAUUUAAAAUUA |
| intron1 | | | | GGACTCAATCGTGAG | | GGACUCAAUCGUGAG |
| STMN2_ | + | ATTC | 1819 | TGTTTCCATATTTAA | 3825 | UGUUUCCAUAUUUAA |
| intron1 | | | | AATTAGGACTCAATC | | AAUUAGGACUCAAUC |
| STMN2_ | + | TTTA | 1820 | TTCTGTTTCCATATT | 3826 | UUCUGUUUCCAUAUU |
| intron1 | | | | TAAAATTAGGACTCA | | UAAAAUUAGGACUCA |
| STMN2_ | + | ATTT | 1821 | ATTCTGTTTCCATAT | 3827 | AUUCUGUUUCCAUAU |
| intron1 | | | | TTAAAATTAGGACTC | | UUAAAAUUAGGACUC |
| STMN2_ | + | GTTG | 1822 | CCCTCCTATGGGTAG | 3828 | CCCUCCUAUGGGUAG |
| intron1 | | | | AGAATTTATTCTGTT | | AGAAUUUAUUCUGUU |
| STMN2_ | + | TTTA | 1823 | AAAGGTAGAAGCGGG | 3829 | AAAGGUAGAAGCGGG |
| intron1 | | | | TAAGTTGCCCTCCTA | | UAAGUUGCCCUCCUA |
| STMN2_ | + | TTTT | 1824 | AAAAGGTAGAAGCGG | 3830 | AAAAGGUAGAAGCGG |
| intron1 | | | | GTAAGTTGCCCTCCT | | GUAAGUUGCCCUCCU |
| STMN2_ | + | CTTT | 1825 | TAAAAGGTAGAAGCG | 3831 | UAAAAGGUAGAAGCG |
| intron1 | | | | GGTAAGTTGCCCTCC | | GGUAAGUUGCCCUCC |
| STMN2_ | + | TTTC | 1826 | TTTTAAAAGGTAGAA | 3832 | UUUUAAAAGGUAGAA |
| intron1 | | | | GCGGGTAAGTTGCCC | | GCGGGUAAGUUGCCC |
| STMN2_ | + | ATTT | 1827 | CTTTTAAAAGGTAGA | 3833 | CUUUUAAAAGGUAGA |
| intron1 | | | | AGCGGGTAAGTTGCC | | AGCGGGUAAGUUGCC |
| STMN2_ | + | GTTC | 1828 | TGGGGGAGGTGGGAG | 3834 | UGGGGGAGGUGGGAG |
| intron1 | | | | GGCAGAGAAGAGGTC | | GGCAGAGAAGAGGUC |
| STMN2_ | + | GTTA | 1829 | ATGGTAACACAGGAC | 3835 | AUGGUAACACAGGAC |
| intron1 | | | | CAGGAAGGACAGGGC | | CAGGAAGGACAGGGC |
| STMN2_ | + | TTTA | 1830 | TAAAGAAAAAGATGT | 3836 | UAAAGAAAAAGAUGU |
| intron1 | | | | TAATGGTAACACAGG | | UAAUGGUAACACAGG |
| STMN2_ | + | TTTT | 1831 | ATAAAGAAAAAGATG | 3837 | AUAAAGAAAAAGAUG |
| intron1 | | | | TTAATGGTAACACAG | | UUAAUGGUAACACAG |
| STMN2_ | + | ATTT | 1832 | TATAAAGAAAAAGAT | 3838 | UAUAAAGAAAAAGAU |
| intron1 | | | | GTTAATGGTAACACA | | GUUAAUGGUAACACA |
| STMN2_ | + | ATTC | 1833 | AGAGATATTTTATAA | 3839 | AGAGAUAUUUUAUAA |
| intron1 | | | | AGAAAAAGATGTTAA | | AGAAAAAGAUGUUAA |
| STMN2_ | + | CTTG | 1834 | AGCTCTAGAAGCATT | 3840 | AGCUCUAGAAGCAUU |
| intron1 | | | | CAGAGATATTTTATA | | CAGAGAUAUUUUAUA |
| STMN2_ | + | ATTA | 1835 | TGAGAACAAAAATAA | 3841 | UGAGAACAAAAAUAA |
| intron1 | | | | AAATGTTCCTCACCC | | AAAUGUUCCUCACCC |
| STMN2_ | + | ATTT | 1836 | TGGAAAGTGCTTTAT | 3842 | UGGAAAGUGCUUUAU |
| intron1 | | | | AACGACCTTTTTTTT | | AACGACCUUUUUUUU |
| STMN2_ | 1 | TTTT | 1837 | GGAAAGTGCTTTATA | 3843 | GGAAAGUGCUUUAUA |
| intron1 | | | | ACGACCTTTTTTTTT | | ACGACCUUUUUUUUU |
| STMN2_ | + | TTTG | 1838 | GAAAGTGCTTTATAA | 3844 | GAAAGUGCUUUAUAA |
| intron1 | | | | CGACCTTTTTTTTTT | | CGACCUUUUUUUUUU |
| STMN2_ | + | CTTT | 1839 | ATAACGACCTTTTTT | 3845 | AUAACGACCUUUUUU |
| intron1 | | | | TTTTTTATTTCTTCT | | UUUUUUAUUUCUUCU |
| STMN2_ | + | TTTT | 1840 | AGGGCAAGGGAGGGG | 3846 | AGGGCAAGGGAGGGG |
| intron1 | | | | AAGGAGAGAGGAAGT | | AAGGAGAGAGGAAGU |
| STMN2_ | + | GTTT | 1841 | TAGGGCAAGGGAGGG | 3847 | UAGGGCAAGGGAGGG |
| intron1 | | | | GAAGGAGAGAGGAAG | | GAAGGAGAGAGGAAG |
| STMN2_ | + | TTTG | 1842 | TTTTAGGGCAAGGGA | 3848 | UUUUAGGGCAAGGGA |
| intron1 | | | | GGGGAAGGAGAGAGG | | GGGGAAGGAGAGAGG |
| STMN2_ | + | CTTT | 1843 | GTTTTAGGGCAAGGG | 3849 | GUUUUAGGGCAAGGG |
| intron1 | | | | AGGGGAAGGAGAGAG | | AGGGGAAGGAGAGAG |
| STMN2_ | + | ATTG | 1844 | TCTCGTCGAAGAAAC | 3850 | UCUCGUCGAAGAAAC |
| intron1 | | | | CGCTAGTCCTGGGGT | | CGCUAGUCCUGGGGU |
| STMN2_ | + | TTTA | 1845 | CGGTATTGTCTCGTC | 3851 | CGGUAUUGUCUCGUC |
| intron1 | | | | GAAGAAACCGCTAGT | | GAAGAAACCGCUAGU |
| STMN2_ | + | TTTT | 1846 | ACGGTATTGTCTCGT | 3852 | ACGGUAUUGUCUCGU |
| intron1 | | | | CGAAGAAACCGCTAG | | CGAAGAAACCGCUAG |
| STMN2_ | + | ATTT | 1847 | TACGGTATTGTCTCG | 3853 | UACGGUAUUGUCUCG |
| intron1 | | | | TCGAAGAAACCGCTA | | UCGAAGAAACCGCUA |
| STMN2_ | + | TTTG | 1848 | AAAGATGGGTGGAGA | 3854 | AAAGAUGGGUGGAGA |
| intron1 | | | | CGGGGGGAGGGGATG | | CGGGGGGAGGGGAUG |
| STMN2_ | + | GTTT | 1849 | GAAAGATGGGTGGAG | 3855 | GAAAGAUGGGUGGAGA |
| intron1 | | | | ACGGGGGGAGGGGAT | | CGGGGGGAGGGGAU |
| STMN2_ | + | ATTG | 1850 | CAAAGTCAAAGCGGT | 3856 | CAAAGUCAAAGCGGUC |
| intron1 | | | | CCCATCCCGCTGTTT | | CCAUCCCGCUGUUU |
| STMN2_ | + | TTTA | 1851 | AGAAGAAAATAGGAA | 3857 | AGAAGAAAAUAGGAAA |
| intron1 | | | | AGGGGTAAAGGGAAG | | GGGGUAAAGGGAAG |
| STMN2_ | + | GTTT | 1852 | AAGAAGAAAATAGGA | 3858 | AAGAAGAAAAUAGGAA |
| intron1 | | | | AAGGGGTAAAGGGAA | | AGGGGUAAAGGGAA |
| STMN2_ | + | TTTT | 1853 | TTTCCCCCAGCCCAA | 3859 | UUUCCCCCAGCCCAAG |
| intron1 | | | | GCCCCCCGCCCACCC | | CCCCCCGCCCACCC |
| STMN2_ | + | CTTC | 1854 | TCTAGTTTAAGAAGA | 3860 | UCUAGUUUAAGAAGAA |
| intron1 | | | | AAATAGGAAAGGGGT | | AAUAGGAAAGGGGU |
| STMN2_ | + | ATTT | 1855 | CTTCTCTAGTTTAAGA | 3861 | CUUCUCUAGUUUAAGA |
| intron1 | | | | AGAAAATAGGAAAG | | AGAAAAUAGGAAAG |
| STMN2_ | + | TTTA | 1856 | TTTCTTCTCTAGTTTA | 3862 | UUUCUUCUCUAGUUUA |
| intron1 | | | | AGAAGAAAATAGGA | | AGAAGAAAAUAGGA |
| STMN2_ | + | TTTT | 1857 | ATTTCTTCTCTAGTT | 3863 | AUUUCUUCUCUAGUUU |
| intron1 | | | | TAAGAAGAAAATAGG | | AAGAAGAAAAUAGG |
| STMN2_ | + | TTTT | 1858 | TATTTCTTCTCTAGT | 3864 | UAUUUCUUCUCUAGUU |
| intron1 | | | | TTAAGAAGAAAATAG | | UAAGAAGAAAAUAG |
| STMN2_ | + | TTTT | 1859 | TTATTTCTTCTCTAG | 3865 | UUAUUUCUUCUCUAG |
| intron1 | | | | TTTAAGAAGAAAATA | | UUUAAGAAGAAAAUA |
| STMN2_ | + | TTTT | 1860 | TTTATTTCTTCTCTA | 3866 | UUUAUUUCUUCUCUAG |
| intron1 | | | | GTTTAAGAAGAAAAT | | UUUAAGAAGAAAAU |
| STMN2_ | + | TTTT | 1861 | TTTTATTTCTTCTCT | 3867 | UUUUAUUUCUUCUCUA |
| intron1 | | | | AGTTTAAGAAGAAAA | | GUUUAAGAAGAAAA |
| STMN2_ | + | TTTT | 1862 | TTTTTATTTCTTCTC | 3868 | UUUUUAUUUCUUCUCU |
| intron1 | | | | TAGTTTAAGAAGAAA | | AGUUUAAGAAGAAA |
| STMN2_ | + | TTTT | 1863 | TTTTTTATTTCTTCT | 3869 | UUUUUUAUUUCUUCUC |
| intron1 | | | | CTAGTTTAAGAAGAA | | UAGUUUAAGAAGAA |
| STMN2_ | + | TTTT | 1864 | TTTTTTTATTTCTTC | 3870 | UUUUUUUAUUUCUUC |
| intron1 | | | | TCTAGTTTAAGAAGA | | UCUAGUUUAAGAAGA |
| STMN2_ | + | TTTT | 1865 | TTTTTTTTATTTCTT | 3871 | UUUUUUUUAUUUCUU |
| intron1 | | | | CTCTAGTTTAAGAAG | | CUCUAGUUUAAGAAG |
| STMN2_ | + | CTTT | 1866 | TTTTTTTTTATTTCT | 387 | UUUUUUUUUAUUUCUU |
| intron1 | | | | TCTCTAGTTTAAGAA | 2 | CUCUAGUUUAAGAA |
| STMN2_ | + | TTTA | 1867 | TAACGACCTTTTTTT | 3873 | UAACGACCUUUUUUUU |
| intron1 | | | | TTTTTATTTCTTCTC | | UUUUAUUUCUUCUC |
| STMN2_ | + | TTTC | 1868 | TTCTCTAGTTTAAGA | 387 | UUCUCUAGUUUAAGAA |
| intron1 | | | | AGAAAATAGGAAAGG | 4 | GAAAAUAGGAAAGG |
| STMN2_ | + | TTTT | 1869 | TTCCCCCAGCCCAAG | 3875 | UUCCCCCAGCCCAAG |
| intron1 | | | | CCCCCCGCCCACCCT | | CCCCCCGCCCACCCU |
| STMN2_ | + | TTTT | 1870 | TCCCCCAGCCCAAGC | 3876 | UCCCCCAGCCCAAGC |
| intron1 | | | | CCCCCGCCCACCCTC | | CCCCCGCCCACCCUC |
| STMN2_ | + | TTTT | 1871 | CCCCCAGCCCAAGCC | 3877 | CCCCCAGCCCAAGCC |
| intron1 | | | | CCCCGCCCACCCTCT | | CCCCGCCCACCCUCU |
| STMN2_ | + | TTTC | 1872 | TGGCCATAATTTAAC | 3878 | UGGCCAUAAUUUAAC |
| intron1 | | | | TGCATTTGCAAATCA | | UGCAUUUGCAAAUCA |
| STMN2_ | + | CTTT | 1873 | CTGGCCATAATTTAA | 3879 | CUGGCCAUAAUUUAA |
| intron1 | | | | CTGCATTTGCAAATC | | CUGCAUUUGCAAAUC |
| STMN2_ | + | CTTG | 1874 | ATACAGCCTCAATCCT | 3880 | AUACAGCCUCAAUCCU |
| intron1 | | | | ACACAGATACATGG | | ACACAGAUACAUGG |
| STMN2_ | + | ATTC | 1875 | TTGATACAGCCTCAAT | 3881 | UUGAUACAGCCUCAAU |
| intron1 | | | | CCTACACAGATACA | | CCUACACAGAUACA |
| STMN2_ | + | CTTC | 1876 | CAACTGCTGATTCTTG | 3882 | CAACUGCUGAUUCUUG |
| intron1 | | | | ATACAGCCTCAATC | | AUACAGCCUCAAUC |
| STMN2_ | + | GTTC | 1877 | TTCCAACTGCTGATT | 3883 | UUCCAACUGCUGAUUC |
| intron1 | | | | CTTGATACAGCCTCA | | UUGAUACAGCCUCA |
| STMN2_ | + | TTTC | 1878 | CCCTGAAACTGTTCT | 3884 | CCCUGAAACUGUUCUU |
| intron1 | | | | TCCAACTGCTGATTC | | CCAACUGCUGAUUC |
| STMN2_ | + | TTTT | 1879 | CCCCTGAAACTGTTC | 3885 | CCCCUGAAACUGUUC |
| intron1 | | | | TTCCAACTGCTGATT | | UUCCAACUGCUGAUU |
| STMN2_ | + | TTTT | 1880 | TCCCCTGAAACTGTT | 3886 | UCCCCUGAAACUGUUC |
| intron1 | | | | CTTCCAACTGCTGAT | | UUCCAACUGCUGAU |
| STMN2_ | + | TTTT | 1881 | TTCCCCTGAAACTGT | 3887 | UUCCCCUGAAACUGUU |
| intron1 | | | | TCTTCCAACTGCTGA | | CUUCCAACUGCUGA |
| STMN2_ | + | GTTT | 1882 | TTTCCCCTGAAACTGT | 3888 | UUUCCCCUGAAACUGU |
| intron1 | | | | TCTTCCAACTGCTG | | UCUUCCAACUGCUG |
| STMN2_ | + | TTTA | 1883 | AGTTTTTTCCCCTGAA | 3889 | AGUUUUUUCCCCUGAA |
| intron1 | | | | ACTGTTCTTCCAAC | | ACUGUUCUUCCAAC |
| STMN2_ | + | TTTT | 1884 | AAGTTTTTTCCCCTG | 3890 | AAGUUUUUUCCCCUGA |
| intron1 | | | | AAACTGTTCTTCCAA | | AACUGUUCUUCCAA |
| STMN2_ | + | ATTT | 1885 | TAAGTTTTTTCCCCT | 3891 | UAAGUUUUUUCCCCU |
| intron1 | | | | GAAACTGTTCTTCCA | | GAAACUGUUCUUCCA |
| STMN2_ | + | TTTA | 1886 | TGCACAAAATTTTAA | 3892 | UGCACAAAAUUUUAA |
| intron1 | | | | GTTTTTTCCCCTGAA | | GUUUUUUCCCCUGAA |
| STMN2_ | + | GTTT | 1887 | ATGCACAAAATTTTA | 3893 | AUGCACAAAAUUUUA |
| intron1 | | | | AGTTTTTTCCCCTGA | | AGUUUUUUCCCCUGA |
| STMN2_ | + | GTTA | 1888 | TATCTATAAATATAT | 3894 | UAUCUAUAAAUAUAUA |
| intron1 | | | | AAATATAGTTTATGC | | AAUAUAGUUUAUGC |
| STMN2_ | + | CTTC | 1889 | AACATAAGGTTATAT | 3895 | AACAUAAGGUUAUAUC |
| intron1 | | | | CTATAAATATATAAA | | UAUAAAUAUAUAAA |
| STMN2_ | + | ATTG | 1890 | AGATGATCTTCAACA | 3896 | AGAUGAUCUUCAACA |
| intron1 | | | | TAAGGTTATATCTAT | | UAAGGUUAUAUCUAU |
| STMN2_ | + | TTTG | 1891 | TGGCTGCAATGGGTG | 3897 | UGGCUGCAAUGGGUG |
| intron1 | | | | AGAATACACATATAT | | AGAAUACACAUAUAU |
| STMN2_ | + | GTTT | 1892 | GTGGCTGCAATGGGT | 3898 | GUGGCUGCAAUGGGU |
| intron1 | | | | GAGAATACACATATA | | GAGAAUACACAUAUA |
| STMN2_ | + | ATTG | 1893 | TTTGTGGCTGCAATGG | 3899 | UUUGUGGCUGCAAUGG |
| intron1 | | | | GTGAGAATACACAT | | GUGAGAAUACACAU |
| STMN2_ | + | ATTC | 1894 | TCTGCAAAGAATTGTT | 3900 | UCUGCAAAGAAUUGUU |
| intron1 | | | | TGTGGCTGCAATGG | | UGUGGCUGCAAUGG |
| STMN2_ | + | ATTG | 1895 | CTGGAAAATTCTCTGC | 3901 | CUGGAAAAUUCUCUGC |
| intron1 | | | | AAAGAATTGTTTGT | | AAAGAAUUGUUUGU |
| STMN2_ | + | TTTG | 1896 | TGTGCCAACGATTGCT | 3902 | UGUGCCAACGAUUGCU |
| intron1 | | | | GGAAAATTCTCTGC | | GGAAAAUUCUCUGC |
| STMN2_ | + | GTTT | 1897 | GTGTGCCAACGATTGC | 3903 | GUGUGCCAACGAUUGC |
| intron1 | | | | TGGAAAATTCTCTG | | UGGAAAAUUCUCUG |
| STMN2_ | + | CTTG | 1898 | CTAAGAGCAGGGTTT | 3904 | CUAAGAGCAGGGUUUG |
| intron1 | | | | GTGTGCCAACGATTG | | UGUGCCAACGAUUG |
| STMN2_ | + | ATTT | 1899 | AACTGCATTTGCAAAT | 3905 | AACUGCAUUUGCAAAU |
| intron1 | | | | CATGAAAAAAACAC | | CAUGAAAAAAACAC |
| STMN2_ | + | ATTT | 1900 | GCAAATCATGAAAAA | 3906 | GCAAAUCAUGAAAAAA |
| intron1 | | | | AACACTACTTCTGCA | | ACACUACUUCUGCA |
| STMN2_ | + | TTTG | 1901 | CAAATCATGAAAAAA | 3907 | CAAAUCAUGAAAAAAA |
| intron1 | | | | ACACTACTTCTGCAG | | CACUACUUCUGCAG |
| STMN2_ | + | CTTC | 1902 | TGCAGTATTAAAATA | 3908 | UGCAGUAUUAAAAUAA |
| intron1 | | | | ATAGATTTTGAAATT | | UAGAUUUUGAAAUU |
| STMN2_ | + | TTTG | 1903 | GTCAGAATTTCAGGAT | 3909 | GUCAGAAUUUCAGGAU |
| intron1 | | | | AAAACTGAAAGAAA | | AAAACUGAAAGAAA |
| STMN2_ | + | ATTT | 1904 | GGTCAGAATTTCAGG | 3910 | GGUCAGAAUUUCAGGA |
| intron1 | | | | ATAAAACTGAAAGAA | | UAAAACUGAAAGAA |
| STMN2_ | + | CTTC | 1905 | TGAATGGATATATAA | 3911 | UGAAUGGAUAUAUAAG |
| intron1 | | | | GTAACTAGAAATGAA | | UAACUAGAAAUGAA |
| STMN2_ | + | TTTC | 1906 | TAATGAAGTGGGCAC | 3912 | UAAUGAAGUGGGCACC |
| intron1 | | | | CTTCTGAATGGATAT | | UUCUGAAUGGAUAU |
| STMN2_ | + | TTTT | 1907 | CTAATGAAGTGGGCA | 3913 | CUAAUGAAGUGGGCAC |
| intron1 | | | | CCTTCTGAATGGATA | | CUUCUGAAUGGAUA |
| STMN2_ | + | CTTT | 1908 | TCTAATGAAGTGGGC | 3914 | UCUAAUGAAGUGGGCA |
| intron1 | | | | ACCTTCTGAATGGAT | | CCUUCUGAAUGGAU |
| STMN2_ | + | ATTA | 1909 | TCTTTTCTAATGAAG | 3915 | UCUUUUCUAAUGAAGU |
| intron1 | | | | TGGGCACCTTCTGAA | | GGGCACCUUCUGAA |
| STMN2_ | + | ATT | 1910 | CCCATTATCTTTTCTA | 3916 | CCCAUUAUCUUUUCUA |
| intron1 | | C | | ATGAAGTGGGCACC | | AUGAAGUGGGCACC |
| STMN2_ | + | ATTG | 1911 | TAAGAGGTGCATATA | 3917 | UAAGAGGUGCAUAUAA |
| intron1 | | | | ATATTCCCCATTATC | | UAUUCCCCAUUAUC |
| STMN2_ | + | ATTC | 1912 | AGCATGATTGTAAGA | 3918 | AGCAUGAUUGUAAGAG |
| intron1 | | | | GGTGCATATAATATT | | GUGCAUAUAAUAUU |
| STMN2_ | + | ATTA | 1913 | TGTATTCAGCATGAT | 3919 | UGUAUUCAGCAUGAU |
| intron1 | | | | TGTAAGAGGTGCATA | | UGUAAGAGGUGCAUA |
| STMN2_ | + | ATTA | 1914 | AACAATTATGTATTC | 3920 | AACAAUUAUGUAUUC |
| intron1 | | | | AGCATGATTGTAAGA | | AGCAUGAUUGUAAGA |
| STMN2_ | + | ATTC | 1915 | ATTAAACAATTATGT | 3921 | AUUAAACAAUUAUGU |
| intron1 | | | | ATTCAGCATGATTGT | | AUUCAGCAUGAUUGU |
| STMN2_ | + | GTTC | 1916 | ATGTGGCTAAGATAC | 3922 | AUGUGGCUAAGAUAC |
| intron1 | | | | ATGTGCAAGTGCTTG | | AUGUGCAAGUGCUUG |
| STMN2_ | + | TTTC | 1917 | CTGATTCATTAAACA | 3923 | CUGAUUCAUUAAACA |
| intron1 | | | | ATTATGTATTCAGCA | | AUUAUGUAUUCAGCA |
| STMN2_ | + | TTTT | 1918 | TCCTGATTCATTAAA | 3924 | UCCUGAUUCAUUAAA |
| intron1 | | | | CAATTATGTATTCAG | | CAAUUAUGUAUUCAG |
| STMN2_ | + | TTTT | 1919 | TTCCTGATTCATTAA | 3925 | UUCCUGAUUCAUUAA |
| intron1 | | | | ACAATTATGTATTCA | | ACAAUUAUGUAUUCA |
| STMN2_ | + | CTTT | 1920 | TTTCCTGATTCATTA | 3926 | UUUCCUGAUUCAUUA |
| intron1 | | | | AACAATTATGTATTC | | AACAAUUAUGUAUUC |
| STMN2_ | + | ATTA | 1921 | TCAGGGCGAGTGCTT | 3927 | UCAGGGCGAGUGCUU |
| intron1 | | | | TTTTCCTGATTCATT | | UUUUCCUGAUUCAUU |
| STMN2_ | + | ATTA | 1922 | ATTATCAGGGCGAGT | 3928 | AUUAUCAGGGCGAGU |
| intron1 | | | | GCTTTTTTCCTGATT | | GCUUUUUUCCUGAUU |
| STMN2_ | + | TTTC | 1923 | AAAGATAATTAATTA | 3929 | AAAGAUAAUUAAUUA |
| intron1 | | | | TCAGGGCGAGTGCTT | | UCAGGGCGAGUGCUU |
| STMN2_ | + | ATTT | 1924 | CAAAGATAATTAATT | 3930 | CAAAGAUAAUUAAUU |
| intron1 | | | | ATCAGGGCGAGTGCT | | AUCAGGGCGAGUGCU |
| STMN2_ | + | ATTC | 1925 | CAATTTCAAAGATAA | 3931 | CAAUUUCAAAGAUAA |
| intron1 | | | | TTAATTATCAGGGCG | | UUAAUUAUCAGGGCG |
| STMN2_ | + | ATTA | 1926 | ATTCCAATTTCAAAG | 3932 | AUUCCAAUUUCAAAG |
| intron1 | | | | ATAATTAATTATCAG | | AUAAUUAAUUAUCAG |
| STMN2_ | + | TTTG | 1927 | AAATTAATTCCAATT | 3933 | AAAUUAAUUCCAAUU |
| intron1 | | | | TCAAAGATAATTAAT | | UCAAAGAUAAUUAAU |
| STMN2_ | + | TTTT | 1928 | GAAATTAATTCCAAT | 3934 | GAAAUUAAUUCCAAU |
| intron1 | | | | TTCAAAGATAATTAA | | UUCAAAGAUAAUUAA |
| STMN2_ | + | ATTT | 1929 | TGAAATTAATTCCAA | 3935 | UGAAAUUAAUUCCAA |
| intron1 | | | | TTTCAAAGATAATTA | | UUUCAAAGAUAAUUA |
| STMN2_ | + | ATTA | 1930 | AAATAATAGATTTTG | 3936 | AAAUAAUAGAUUUUG |
| intron1 | | | | AAATTAATTCCAATT | | AAAUUAAUUCCAAUU |
| STMN2_ | + | TTTT | 1931 | CCTGATTCATTAAAC | 3937 | CCUGAUUCAUUAAAC |
| intron1 | | | | AATTATGTATTCAGC | | AAUUAUGUAUUCAGC |
| STMN2_ | + | TTTC | 1932 | AGGATAAAACTGAAA | 3938 | AGGAUAAAACUGAAA |
| intron1 | | | | GAAATGGCAGTAGTT | | GAAAUGGCAGUAGUU |
| STMN2_ | + | ATTA | 1933 | TGTATGTTCATGTGG | 3939 | UGUAUGUUCAUGUGG |
| intron1 | | | | CTAAGATACATGTGC | | CUAAGAUACAUGUGC |
| STMN2_ | + | TTTG | 1934 | CTTCCTGCCAGGATT | 3940 | CUUCCUGCCAGGAUU |
| intron1 | | | | ATGTATGTTCATGTG | | AUGUAUGUUCAUGUG |
| STMN2_ | + | TTTA | 1935 | ATGATTCAGTAGCCT | 3941 | AUGAUUCAGUAGCCU |
| intron1 | | | | TGTTTGTTCTCATTT | | UGUUUGUUCUCAUUU |
| STMN2_ | + | ATTT | 1936 | AATGATTCAGTAGCC | 3942 | AAUGAUUCAGUAGCC |
| intron1 | | | | TTGTTTGTTCTCATT | | UUGUUUGUUCUCAUU |
| STMN2_ | + | ATTA | 1937 | TTTAATGATTCAGTA | 3943 | UUUAAUGAUUCAGUA |
| intron1 | | | | GCCTTGTTTGTTCTC | | GCCUUGUUUGUUCUC |
| STMN2_ | + | GTTA | 1938 | TTATTTAATGATTCA | 3944 | UUAUUUAAUGAUUCA |
| intron1 | | | | GTAGCCTTGTTTGTT | | GUAGCCUUGUUUGUU |
| STMN2_ | + | GTTG | 1939 | TTATTATTTAATGAT | 3945 | UUAUUAUUUAAUGAU |
| intron1 | | | | TCAGTAGCCTTGTTT | | UCAGUAGCCUUGUUU |
| STMN2_ | + | TTTC | 1940 | AAATCGTTGTTATTA | 3946 | AAAUCGUUGUUAUUA |
| intron1 | | | | TTTAATGATTCAGTA | | UUUAAUGAUUCAGUA |
| STMN2_ | 1 | ATTT | 1941 | CAAATCGTTGTTATT | 3947 | CAAAUCGUUGUUAUU |
| intron1 | | | | ATTTAATGATTCAGT | | AUUUAAUGAUUCAGU |
| STMN2_ | + | ATTA | 1942 | TCATTTCAAATCGTT | 3948 | UCAUUUCAAAUCGUU |
| intron1 | | | | GTTATTATTTAATGA | | GUUAUUAUUUAAUGA |
| STMN2_ | + | ATTA | 1943 | TTATCATTTCAAATC | 3949 | UUAUCAUUUCAAAUC |
| intron1 | | | | GTTGTTATTATTTAA | | GUUGUUAUUAUUUAA |
| STMN2_ | + | TTTC | 1944 | CGCTGCAGGCTAGTG | 3950 | CGCUGCAGGCUAGUG |
| intron1 | | | | GCTGCAAACTCATCG | | GCUGCAAACUCAUCG |
| STMN2_ | + | GTTT | 1945 | CCGCTGCAGGCTAGT | 3951 | CCGCUGCAGGCUAGU |
| intron1 | | | | GGCTGCAAACTCATC | | GGCUGCAAACUCAUC |
| STMN2_ | + | TTTC | 1946 | TGCACCCCTCAGAAA | 3952 | UGCACCCCUCAGAAA |
| intron1 | | | | GGTTTCCGCTGCAGG | | GGUUUCCGCUGCAGG |
| STMN2_ | + | CTTT | 1947 | CTGCACCCCTCAGAA | 3953 | CUGCACCCCUCAGAA |
| intron1 | | | | AGGTTTCCGCTGCAG | | AGGUUUCCGCUGCAG |
| STMN2_ | + | GTTC | 1948 | TGAGAATGGGTGGTG | 3954 | UGAGAAUGGGUGGUG |
| intron1 | | | | GGGGCGATCTCGCCT | | GGGGCGAUCUCGCCU |
| STMN2_ | + | GTTC | 1949 | ACTCGCACGTCCAGA | 3955 | ACUCGCACGUCCAGA |
| intron1 | | | | AAGGTTCTGAGAATG | | AAGGUUCUGAGAAUG |
| STMN2_ | + | ATTC | 1950 | GCAGTTCACTCGCAC | 3956 | GCAGUUCACUCGCAC |
| intron1 | | | | GTCCAGAAAGGTTCT | | GUCCAGAAAGGUUCU |
| STMN2_ | + | TTTC | 1951 | CACAATTCGCAGTTC | 3957 | CACAAUUCGCAGUUC |
| intron1 | | | | ACTCGCACGTCCAGA | | ACUCGCACGUCCAGA |
| STMN2_ | + | CTTT | 1952 | CCACAATTCGCAGTT | 3958 | CCACAAUUCGCAGUU |
| intron1 | | | | CACTCGCACGTCCAG | | CACUCGCACGUCCAG |
| STMN2_ | + | TTTC | 1953 | TGCGCAGTGTCCTGA | 3959 | UGCGCAGUGUCCUGA |
| intron1 | | | | GCTACCCCCGCTTTC | | GCUACCCCCGCUUUC |
| STMN2_ | + | GTTT | 1954 | CTGCGCAGTGTCCTG | 3960 | CUGCGCAGUGUCCUG |
| intron1 | | | | AGCTACCCCCGCTTT | | AGCUACCCCCGCUUU |
| STMN2_ | + | GTTG | 1955 | GGCGCTCGCCCCCGC | 3961 | GGCGCUCGCCCCCGC |
| intron1 | | | | GGTGCAGCCGGGGAG | | GGUGCAGCCGGGGAG |
| STMN2_ | + | CTTC | 1956 | TCTAAGGGAGACCCT | 3962 | UCUAAGGGAGACCCU |
| intron1 | | | | CGCTCCTCCAGCGGG | | CGCUCCUCCAGCGGG |
| STMN2_ | + | TTTC | 1957 | CAGAATGGAGACCCC | 3963 | CAGAAUGGAGACCCC |
| intron1 | | | | GCGAGGGGCTTCTCT | | GCGAGGGGCUUCUCU |
| STMN2_ | + | TTTT | 1958 | CCAGAATGGAGACCC | 3964 | CCAGAAUGGAGACCC |
| intron1 | | | | CGCGAGGGGCTTCT | | CGCGAGGGGCUUCU |
| | | | | C | | C |
| STMN2_ | + | ATTT | 1959 | TCCAGAATGGAGACC | 3965 | UCCAGAAUGGAGACC |
| intron1 | | | | CCGCGAGGGGCTTC | | CCGCGAGGGGCUUC |
| | | | | T | | U |
| STMN2_ | + | GTTC | 1960 | TCTATGATTTTCCAG | 3966 | UCUAUGAUUUUCCAG |
| intron1 | | | | AATGGAGACCCCGC | | AAUGGAGACCCCGC |
| | | | | G | | G |
| STMN2_ | + | TTTC | 1961 | CCCCAGCCCAAGCCC | 3967 | CCCCAGCCCAAGCCC |
| intron1 | | | | CCCGCCCACCCTCT | | CCCGCCCACCCUCU |
| | | | | G | | G |
| STMN2_ | + | ATTC | 1962 | AGTAGCCTTGTTTGT | 3968 | AGUAGCCUUGUUUGU |
| intron1 | | | | TCTCATTTGTTCAA | | UCUCAUUUGUUCAA |
| | | | | A | | A |
| STMN2_ | + | CTTG | 1963 | TTTGTTCTCATTTGT | 3969 | UUUGUUCUCAUUUGU |
| intron1 | | | | TCAAAAGGGACGTG | | UCAAAAGGGACGUG |
| | | | | G | | G |
| STMN2_ | + | GTTT | 1964 | GTTCTCATTTGTTCA | 3970 | GUUCUCAUUUGUUCA |
| intron1 | | | | AAAGGGACGTGGAT | | AAAGGGACGUGGAU |
| | | | | T | | U |
| STMN2_ | + | TTTG | 1965 | TTCTCATTTGTTCAA | 3971 | UUCUCAUUUGUUCAA |
| intron1 | | | | AAGGGACGTGGATT | | AAGGGACGUGGAUU |
| | | | | G | | G |
| STMN2_ | + | TTTT | 1966 | GCTTCCTGCCAGGAT | 3972 | GCUUCCUGCCAGGAU |
| intron1 | | | | TATGTATGTTCATG | | UAUGUAUGUUCAUG |
| | | | | T | | U |
| STMN2_ | + | TTTT | 1967 | TGCTTCCTGCCAGGA | 3973 | UGCUUCCUGCCAGGA |
| intron1 | | | | TTATGTATGTTCAT | | UUAUGUAUGUUCAU |
| | | | | G | | G |
| STMN2_ | + | ATTT | 1968 | TTGCTTCCTGCCAGG | 3974 | UUGCUUCCUGCCAGG |
| intron1 | | | | ATTATGTATGTTCA | | AUUAUGUAUGUUCA |
| | | | | T | | U |
| STMN2_ | + | GTTA | 1969 | TAAAGCAAATATATT | 3975 | UAAAGCAAAUAUAUU |
| intron1 | | | | TTTGCTTCCTGCCA | | UUUGCUUCCUGCCA |
| | | | | G | | G |
| STMN2_ | + | TTTA | 1970 | AGTTATAAAGCAAAT | 3976 | AGUUAUAAAGCAAAU |
| intron1 | | | | ATATTTTTGCTTCC | | AUAUUUUUGCUUCC |
| | | | | T | | U |
| STMN2_ | + | TTTT | 1971 | AAGTTATAAAGCAAA | 3977 | AAGUUAUAAAGCAAA |
| intron1 | | | | TATATTTTTGCTTC | | UAUAUUUUUGCUUC |
| | | | | C | | C |
| STMN2_ | + | ATTT | 1972 | TAAGTTATAAAGCAA | 3978 | UAAGUUAUAAAGCAA |
| intron1 | | | | ATATATTTTTGCTT | | AUAUAUUUUUGCUU |
| | | | | C | | C |
| STMN2_ | + | GTTC | 1973 | ATTTTAAGTTATAAA | 3979 | AUUUUAAGUUAUAAA |
| intron1 | | | | GCAAATATATTTTT | | GCAAAUAUAUUUUU |
| | | | | G | | G |
| STMN2_ | + | GTTG | 1974 | TTCATTTTAAGTTAT | 3980 | UUCAUUUUAAGUUAU |
| intron1 | | | | AAAGCAAATATATT | | AAAGCAAAUAUAUU |
| | | | | T | | U |
| STMN2_ | + | TTTG | 1975 | TCTCCAGTTGTTCAT | 3981 | UCUCCAGUUGUUCAU |
| intron | | | | TTTAAGTTATAAAG | | UUUAAGUUAUAAAG |
| | | | | C | | C |
| STMN2_ | + | ATTT | 1976 | GTCTCCAGTTGTTCA | 3982 | GUCUCCAGUUGUUCA |
| intron1 | | | | TTTTAAGTTATAAA | | UUUUAAGUUAUAAA |
| | | | | G | | G |
| STMN2_ | + | TTTC | 1977 | CCCCACAAAAAGGTA | 3983 | CCCCACAAAAAGGUA |
| intron1 | | | | AATTTGTCTCCAGT | | AAUUUGUCUCCAGU |
| | | | | T | | U |
| STMN2_ | + | CTTT | 1978 | CCCCCACAAAAAGGT | 3984 | CCCCCACAAAAAGGU |
| intron1 | | | | AAATTTGTCTCCAG | | AAAUUUGUCUCCAG |
| | | | | T | | U |
| STMN2_ | + | CTTC | 1979 | CTGCCAGGATTATGT | 3985 | CUGCCAGGAUUAUGU |
| intron1 | | | | ATGTTCATGTGGCT | | AUGUUCAUGUGGCU |
| | | | | A | | A |
| STMN2_ | + | TTTT | 1958 | CCAGAATGGAGACCC | 3964 | CCAGAAUGGAGACCC |
| intron1 | | | | CGCGAGGGGCTTCTC | | CGCGAGGGGCUUCUC |
| STMN2_ | + | ATTT | 1959 | TCCAGAATGGAGACC | 3965 | UCCAGAAUGGAGACCC |
| intron1 | | | | CCGCGAGGGGCTTCT | | CGCGAGGGGCUUCU |
| STMN2_ | + | GTTC | 1960 | TCTATGATTTTCCAGA | 3966 | UCUAUGAUUUUCCAGA |
| intron1 | | | | ATGGAGACCCCGCG | | AUGGAGACCCCGCG |
| STMN2_ | + | TTTC | 1961 | CCCCAGCCCAAGCCC | 3967 | CCCCAGCCCAAGCCCCC |
| intron1 | | | | CCCGCCCACCCTCTG | | CGCCCACCCUCUG |
| STMN2_ | + | ATTC | 1962 | AGTAGCCTTGTTTGTT | 3968 | AGUAGCCUUGUUUGUU |
| intron1 | | | | CTCATTTGTTCAAA | | CUCAUUUGUUCAAA |
| STMN2_ | + | CTTG | 1963 | TTTGTTCTCATTTGTTC | 3969 | UUUGUUCUCAUUUGUU |
| intron1 | | | | AAAAGGGACGTGG | | CAAAAGGGACGUGG |
| STMN2_ | + | GTTT | 1964 | GTTCTCATTTGTTCAA | 3970 | GUUCUCAUUUGUUCAA |
| intron1 | | | | AAGGGACGTGGATT | | AAGGGACGUGGAUU |
| STMN2_ | + | TTTG | 1965 | TTCTCATTTGTTCAAA | 3971 | UUCUCAUUUGUUCAAA |
| intron1 | | | | AGGGACGTGGATTG | | AGGGACGUGGAUUG |
| STMN2_ | + | TTTT | 1966 | GCTTCCTGCCAGGAT | 3972 | GCUUCCUGCCAGGAUU |
| intron1 | | | | TATGTATGTTCATGT | | AUGUAUGUUCAUGU |
| STMN2_ | + | TTTT | 1967 | TGCTTCCTGCCAGGA | 3973 | UGCUUCCUGCCAGGAU |
| intron1 | | | | TTATGTATGTTCATG | | UAUGUAUGUUCAUG |
| STMN2_ | + | ATTT | 1968 | TTGCTTCCTGCCAGG | 3974 | UUGCUUCCUGCCAGGA |
| intron1 | | | | ATTATGTATGTTCAT | | UUAUGUAUGUUCAU |
| STMN2_ | + | GTTA | 1969 | TAAAGCAAATATATTT | 3975 | UAAAGCAAAUAUAUUU |
| intron1 | | | | TTGCTTCCTGCCAG | | UUGCUUCCUGCCAG |
| STMN2_ | + | TTTA | 1970 | AGTTATAAAGCAAAT | 3976 | AGUUAUAAAGCAAAUA |
| intron1 | | | | ATATTTTTGCTTCCT | | UAUUUUUGCUUCCU |
| STMN2_ | + | TTTT | 1971 | AAGTTATAAAGCAAA | 3977 | AAGUUAUAAAGCAAAU |
| intron1 | | | | TATATTTTTGCTTCC | | AUAUUUUUGCUUCC |
| STMN2_ | + | ATTT | 1972 | TAAGTTATAAAGCAA | 3978 | UAAGUUAUAAAGCAAA |
| intron1 | | | | ATATATTTTTGCTTC | | UAUAUUUUUGCUUC |
| STMN2_ | + | GTTC | 1973 | ATTTTAAGTTATAAA | 3979 | AUUUUAAGUUAUAAAG |
| intron1 | | | | GCAAATATATTTTTG | | CAAAUAUAUUUUUG |
| STMN2_ | + | GTTG | 1974 | TTCATTTTAAGTTATA | 3980 | UUCAUUUUAAGUUAUA |
| intron1 | | | | AAGCAAATATATTT | | AAGCAAAUAUAUUU |
| STMN2_ | + | TTTG | 1975 | TCTCCAGTTGTTCATT | 3981 | UCUCCAGUUGUUCAUU |
| intron1 | | | | TTAAGTTATAAAGC | | UUAAGUUAUAAAGC |
| STMN2_ | + | ATTT | 1976 | GTCTCCAGTTGTTCAT | 3982 | GUCUCCAGUUGUUCAU |
| intron1 | | | | TTTAAGTTATAAAG | | UUUAAGUUAUAAAG |
| STMN2_ | + | TTTC | 1977 | CCCCACAAAAAGGTA | 3983 | CCCCACAAAAAGGUAA |
| intron1 | | | | AATTTGTCTCCAGTT | | AUUUGUCUCCAGUU |
| STMN2_ | + | CTTT | 1978 | CCCCCACAAAAAGGT | 3984 | CCCCCACAAAAAGGUA |
| intron1 | | | | AAATTTGTCTCCAGT | | AAUUUGUCUCCAGU |
| STMN2_ | + | CTTC | 1979 | CTGCCAGGATTATGT | 3985 | CUGCCAGGAUUAUGUA |
| intron1 | | | | ATGTTCATGTGGCTA | | UGUUCAUGUGGCUA |
| STMN2_ | + | ATTG | 1980 | CCCTCATCCCTTTCC | 3986 | CCCUCAUCCCUUUCC |
| intron1 | | | | CCCACAAAAAGGTAA | | CCCACAAAAAGGUAA |
| STMN2_ | + | TTTG | 1981 | CTTCCTCCTAATTGCC | 3987 | CUUCCUCCUAAUUGCC |
| intron1 | | | | CTCATCCCTTTCCC | | CUCAUCCCUUUCCC |
| STMN2_ | + | CTTT | 1982 | GCTTCCTCCTAATTGC | 3988 | GCUUCCUCCUAAUUG |
| intron1 | | | | CCTCATCCCTTTCC | | CCCUCAUCCCUUUCC |
| STMN2_ | + | GTTC | 1983 | GCTTTGCTTCCTCCT | 3989 | GCUUUGCUUCCUCCUA |
| intron1 | | | | AATTGCCCTCATCCC | | AUUGCCCUCAUCCC |
| STMN2_ | + | GTTG | 1984 | CGTTCGCTTTGCTTCC | 3990 | CGUUCGCUUUGCUUCC |
| intron1 | | | | TCCTAATTGCCCTC | | UCCUAAUUGCCCUC |
| STMN2_ | + | CTTG | 1985 | TTGCGTTCGCTTTGCT | 3991 | UUGCGUUCGCUUUGCU |
| intron1 | | | | TCCTCCTAATTGCC | | UCCUCCUAAUUGCC |
| STMN2_ | + | ATTA | 1986 | ACCCTTGTTGCGTTCG | 3992 | ACCCUUGUUGCGUUCG |
| intron1 | | | | CTTTGCTTCCTCCT | | CUUUGCUUCCUCCU |
| STMN2_ | + | GTTA | 1987 | AGGATTAACCCTTGTT | 3993 | AGGAUUAACCCUUGUU |
| intron1 | | | | GCGTTCGCTTTGCT | | GCGUUCGCUUUGCU |
| STMN2_ | + | CTTG | 1988 | GTTAAGGATTAACCCT | 3994 | GUUAAGGAUUAACCCU |
| intron1 | | | | TGTTGCGTTCGCTT | | UGUUGCGUUCGCUU |
| STMN2_ | + | ATTG | 1989 | CTCTTGGTTAAGGATT | 3995 | CUCUUGGUUAAGGAUU |
| intron1 | | | | AACCCTTGTTGCGT | | AACCCUUGUUGCGU |
| STMN2_ | + | GTTC | 1990 | AAAAGGGACGTGGAT | 3996 | AAAAGGGACGUGGAUU |
| intron1 | | | | TGCTCTTGGTTAAGG | | GCUCUUGGUUAAGG |
| STMN2_ | + | TTTG | 1991 | TTCAAAAGGGACGTG | 3997 | UUCAAAAGGGACGUGG |
| intron1 | | | | GATTGCTCTTGGTTA | | AUUGCUCUUGGUUA |
| STMN2_ | + | ATTT | 1992 | GTTCAAAAGGGACGT | 3998 | GUUCAAAAGGGACGUG |
| intron1 | | | | GGATTGCTCTTGGTT | | GAUUGCUCUUGGUU |
| STMN2_ | + | GTTC | 1993 | TCATTTGTTCAAAAGG | 3999 | UCAUUUGUUCAAAAGG |
| intron1 | | | | GACGTGGATTGCTC | | GACGUGGAUUGCUC |
| STMN2_ | + | CTTC | 1994 | CTCCTAATTGCCCTCA | 4000 | CUCCUAAUUGCCCUCA |
| intron1 | | | | TCCCTTTCCCCCAC | | UCCCUUUCCCCCAC |
| STMN2_ | + | TTTT | 1995 | TGATTACATTTTATG | 4001 | UGAUUACAUUUUAUGU |
| intron1 | | | | TAATTCTAATCCAGC | | AAUUCUAAUCCAGC |
| STMN2_ | + | TTTA | 1996 | ACTGCATTTGCAAATC | 4002 | ACUGCAUUUGCAAAUC |
| intron1 | | | | ATGAAAAAAACACT | | AUGAAAAAAACACU |
| STMN2_ | + | TTTG | 1997 | ATTACATTTTATGTA | 4003 | AUUACAUUUUAUGUAA |
| intron1 | | | | ATTCTAATCCAGCTA | | UUCUAAUCCAGCUA |
| STMN2_ | + | GTTC | 1998 | GCACATTAACCATTAG | 4004 | GCACAUUAACCAUUAG |
| intron1 | | | | TACAAGTACCCAAT | | UACAAGUACCCAAU |
| STMN2_ | + | GTTG | 1999 | GAGTTCGCACATTAA | 4005 | GAGUUCGCACAUUAAC |
| intron1 | | | | CCATTAGTACAAGTA | | CAUUAGUACAAGUA |
| STMN2_ | + | TTTG | 2000 | GATGTTGGAGTTCGCA | 4006 | GAUGUUGGAGUUCGCA |
| intron1 | | | | CATTAACCATTAGT | | CAUUAACCAUUAGU |
| STMN2_ | + | TTTT | 2001 | GGATGTTGGAGTTCG | 4007 | GGAUGUUGGAGUUCG |
| intron1 | | | | CACATTAACCATTAG | | CACAUUAACCAUUAG |
| STMN2_ | + | ATTT | 2002 | TGGATGTTGGAGTTC | 4008 | UGGAUGUUGGAGUUC |
| intron1 | | | | GCACATTAACCATTA | | GCACAUUAACCAUUA |
| STMN2_ | + | ATTG | 2003 | TATTTTGGATGTTGGA | 4009 | UAUUUUGGAUGUUGGA |
| intron1 | | | | GTTCGCACATTAAC | | GUUCGCACAUUAAC |
| STMN2_ | + | CTTC | 2004 | TGGAATAATTGTATTT | 4010 | UGGAAUAAUUGUAUUU |
| intron1 | | | | TGGATGTTGGAGTT | | UGGAUGUUGGAGUU |
| STMN2_ | + | ATTC | 2005 | TTCTGGAATAATTGTA | 4011 | UUCUGGAAUAAUUGUA |
| intron1 | | | | TTTTGGATGTTGGA | | UUUUGGAUGUUGGA |
| STMN2_ | + | GTTA | 2006 | TTCTTCTGGAATAATT | 4012 | UUCUUCUGGAAUAAUU |
| intron1 | | | | GTATTTTGGATGTT | | GUAUUUUGGAUGUU |
| STMN2_ | + | TTTA | 2007 | GCAGTTATTCTTCTGG | 4013 | GCAGUUAUUCUUCUGG |
| intron1 | | | | AATAATTGTATTTT | | AAUAAUUGUAUUUU |
| STMN2_ | + | ATTT | 2008 | AGCAGTTATTCTTCT | 4014 | AGCAGUUAUUCUUCUG |
| intron1 | | | | GGAATAATTGTATTT | | GAAUAAUUGUAUUU |
| STMN2_ | + | ATTG | 2009 | TAAAGCAACGCCTGC | 4015 | UAAAGCAACGCCUGCA |
| intron1 | | | | AAGAGTGCCCATTTA | | AGAGUGCCCAUUUA |
| STMN2_ | + | TTTA | 2010 | CAAAGATTGTAAAGC | 4016 | CAAAGAUUGUAAAGCA |
| intron1 | | | | AACGCCTGCAAGAGT | | ACGCCUGCAAGAGU |
| STMN2_ | + | TTTT | 2011 | ACAAAGATTGTAAAG | 4017 | ACAAAGAUUGUAAAGC |
| intron1 | | | | CAACGCCTGCAAGAG | | AACGCCUGCAAGAG |
| STMN2_ | + | TTTT | 2012 | TACAAAGATTGTAAA | 4018 | UACAAAGAUUGUAAA |
| intron1 | | | | GCAACGCCTGCAAGA | | GCAACGCCUGCAAGA |
| STMN2_ | + | TTTT | 2013 | TTACAAAGATTGTAA | 4019 | UUACAAAGAUUGUAA |
| intron1 | | | | AGCAACGCCTGCAAG | | AGCAACGCCUGCAAG |
| STMN2_ | + | TTTT | 2014 | TTTACAAAGATTGTA | 4020 | UUUACAAAGAUUGUA |
| intron1 | | | | AAGCAACGCCTGCAA | | AAGCAACGCCUGCAA |
| STMN2_ | + | TTTT | 2015 | TTTTACAAAGATTGT | 4021 | UUUUACAAAGAUUGU |
| intron1 | | | | AAAGCAACGCCTGCA | | AAAGCAACGCCUGCA |
| STMN2_ | + | TTTT | 2016 | TTTTTACAAAGATTG | 4022 | UUUUUACAAAGAUUG |
| intron1 | | | | TAAAGCAACGCCTGC | | UAAAGCAACGCCUGC |
| STMN2_ | + | TTTT | 2017 | TTTTTTACAAAGATT | 4023 | UUUUUUACAAAGAUU |
| intron1 | | | | GTAAAGCAACGCCTG | | GUAAAGCAACGCCUG |
| STMN2_ | + | TTTT | 2018 | TTTTTTTACAAAGAT | 4024 | UUUUUUUACAAAGAU |
| intron1 | | | | TGTAAAGCAACGCCT | | UGUAAAGCAACGCCU |
| STMN2_ | + | TTTT | 2019 | TTTTTTTTACAAAGA | 4025 | UUUUUUUUACAAAGA |
| intron1 | | | | TTGTAAAGCAACGCC | | UUGUAAAGCAACGCC |
| STMN2_ | + | TTTT | 2020 | TTTTTTTTTACAAAG | 4026 | UUUUUUUUUACAAAG |
| intron1 | | | | ATTGTAAAGCAACGC | | AUUGUAAAGCAACGC |
| STMN2_ | + | ATTT | 2021 | TTTTTTTTTTACAAA | 4027 | UUUUUUUUUUACAAA |
| intron1 | | | | GATTGTAAAGCAACG | | GAUUGUAAAGCAACG |
| STMN2_ | + | ATTG | 2022 | CCTAGGACTGAATGA | 4028 | CCUAGGACUGAAUGA |
| intron1 | | | | TTTTTTTTTTTTTAC | | UUUUUUUUUUUUUAC |
| STMN2_ | + | TTTA | 2023 | TAGGGCAAAAATATT | 4029 | UAGGGCAAAAAUAUU |
| intron1 | | | | GCCTAGGACTGAATG | | GCCUAGGACUGAAUG |
| STMN2_ | + | TTTT | 2024 | ATAGGGCAAAAATAT | 4030 | AUAGGGCAAAAAUAU |
| intron1 | | | | TGCCTAGGACTGAAT | | UGCCUAGGACUGAAU |
| STMN2_ | + | ATTA | 2025 | ACCATTAGTACAAGT | 4031 | ACCAUUAGUACAAGU |
| intron1 | | | | ACCCAATATAACAAT | | ACCCAAUAUAACAAU |
| STMN2_ | + | TTTT | 2026 | TATAGGGCAAAAATA | 4032 | UAUAGGGCAAAAAUA |
| intron1 | | | | TTGCCTAGGACTGAA | | UUGCCUAGGACUGAA |
| STMN2_ | + | ATTA | 2027 | GTACAAGTACCCAAT | 4033 | GUACAAGUACCCAAU |
| intron1 | | | | ATAACAATAGATCAT | | AUAACAAUAGAUCAU |
| STMN2_ | + | TTTT | 2028 | AGTTGTATGTCTTTA | 4034 | AGUUGUAUGUCUUUA |
| intron1 | | | | TATCAGGATAAAGAG | | UAUCAGGAUAAAGAG |
| STMN2_ | + | TTTC | 2029 | CTTATGAAATGCAGC | 4035 | CUUAUGAAAUGCAGC |
| intron1 | | | | CATAAAGTTTAACTT | | CAUAAAGUUUAACUU |
| STMN2_ | + | TTTT | 2030 | CCTTATGAAATGCAG | 4036 | CCUUAUGAAAUGCAG |
| intron1 | | | | CCATAAAGTTTAACT | | CCAUAAAGUUUAACU |
| STMN2_ | + | TTTT | 2031 | TCCTTATGAAATGCA | 4037 | UCCUUAUGAAAUGCA |
| intron1 | | | | GCCATAAAGTTTAAC | | GCCAUAAAGUUUAAC |
| STMN2_ | + | TTTT | 2032 | TTCCTTATGAAATGC | 4038 | UUCCUUAUGAAAUGC |
| intron1 | | | | AGCCATAAAGTTTAA | | AGCCAUAAAGUUUAA |
| STMN2_ | + | TTTT | 2033 | TTTCCTTATGAAATG | 4039 | UUUCCUUAUGAAAUG |
| intron1 | | | | CAGCCATAAAGTTTA | | CAGCCAUAAAGUUUA |
| STMN2_ | + | TTTT | 2034 | TTTTCCTTATGAAAT | 4040 | UUUUCCUUAUGAAAUG |
| intron1 | | | | GCAGCCATAAAGTTT | | CAGCCAUAAAGUUU |
| STMN2_ | + | GTTT | 2035 | TTTTTCCTTATGAAA | 4041 | UUUUUCCUUAUGAAAU |
| intron1 | | | | TGCAGCCATAAAGTT | | GCAGCCAUAAAGUU |
| STMN2_ | + | TTTG | 2036 | GAAGTTTTTTTTCCTT | 4042 | GAAGUUUUUUUUCCUU |
| intron1 | | | | ATGAAATGCAGCCA | | AUGAAAUGCAGCCA |
| STMN2_ | + | CTTT | 2037 | GGAAGTTTTTTTTCC | 4043 | GGAAGUUUUUUUUCCU |
| intron1 | | | | TTATGAAATGCAGCC | | UAUGAAAUGCAGCC |
| STMN2_ | + | ATTC | 2038 | TACTCTGTCTTTGGAA | 4044 | UACUCUGUCUUUGGAA |
| intron1 | | | | GTTTTTTTTCCTTA | | GUUUUUUUUCCUUA |
| STMN2_ | + | ATTA | 2039 | GCATTCTACTCTGTC | 4045 | GCAUUCUACUCUGUCU |
| intron1 | | | | TTTGGAAGTTTTTTT | | UUGGAAGUUUUUUU |
| STMN2_ | + | TTTA | 2040 | TTAGCATTCTACTCT | 4046 | UUAGCAUUCUACUCUG |
| intron1 | | | | GTCTTTGGAAGTTTT | | UCUUUGGAAGUUUU |
| STMN2_ | + | TTTT | 2041 | ATTAGCATTCTACTC | 4047 | AUUAGCAUUCUACUC |
| intron1 | | | | TGTCTTTGGAAGTTT | | UGUCUUUGGAAGUUU |
| STMN2_ | + | TTTT | 2042 | TATTAGCATTCTACT | 4048 | UAUUAGCAUUCUACU |
| intron1 | | | | CTGTCTTTGGAAGTT | | CUGUCUUUGGAAGUU |
| STMN2_ | + | ATTT | 2043 | TTATTAGCATTCTAC | 4049 | UUAUUAGCAUUCUAC |
| intron1 | | | | TCTGTCTTTGGAAGT | | UCUGUCUUUGGAAGU |
| STMN2_ | + | ATTA | 2044 | AATTTTTATTAGCATT | 4050 | AAUUUUUAUUAGCAUU |
| intron1 | | | | CTACTCTGTCTTTG | | CUACUCUGUCUUUG |
| STMN2_ | + | GTTA | 2045 | AAGTGTAAATTAAATT | 4051 | AAGUGUAAAUUAAAUU |
| intron1 | | | | TTTATTAGCATTCT | | UUUAUUAGCAUUCU |
| STMN2_ | + | TTTA | 2046 | CAAGAGAGCATGTTA | 4052 | CAAGAGAGCAUGUUA |
| intron1 | | | | AAGTGTAAATTAAAT | | AAGUGUAAAUUAAAU |
| STMN2_ | + | TTTT | 2047 | ACAAGAGAGCATGTT | 4053 | ACAAGAGAGCAUGUUA |
| intron1 | | | | AAAGTGTAAATTAAA | | AAGUGUAAAUUAAA |
| STMN2_ | + | CTTT | 2048 | TACAAGAGAGCATGT | 4054 | UACAAGAGAGCAUGU |
| intron1 | | | | TAAAGTGTAAATTAA | | UAAAGUGUAAAUUAA |
| STMN2_ | + | TTTA | 2049 | TCTAAACCTAGTCCC | 4055 | UCUAAACCUAGUCCCA |
| intron1 | | | | ACAAATACTTTTACA | | CAAAUACUUUUACA |
| STMN2_ | + | ATTT | 2050 | ATCTAAACCTAGTCCC | 4056 | AUCUAAACCUAGUCCC |
| intron1 | | | | ACAAATACTTTTAC | | ACAAAUACUUUUAC |
| STMN2_ | + | ATTG | 2051 | AGTGAAATTTATCTAA | 4057 | AGUGAAAUUUAUCUAA |
| intron1 | | | | ACCTAGTCCCACAA | | ACCUAGUCCCACAA |
| STMN2_ | + | TTTA | 2052 | TATCAGGATAAAGAG | 4058 | UAUCAGGAUAAAGAGA |
| intron1 | | | | AATTGAGTGAAATTT | | AUUGAGUGAAAUUU |
| STMN2_ | + | CTTT | 2053 | ATATCAGGATAAAGA | 4059 | AUAUCAGGAUAAAGAG |
| intron1 | | | | GAATTGAGTGAAATT | | AAUUGAGUGAAAUU |
| STMN2_ | + | GTTG | 2054 | TATGTCTTTATATCAG | 4060 | UAUGUCUUUAUAUCAG |
| intron1 | | | | GATAAAGAGAATTG | | GAUAAAGAGAAUUG |
| STMN2_ | + | TTTA | 2055 | GTTGTATGTCTTTATA | 4061 | GUUGUAUGUCUUUAUA |
| intron1 | | | | TCAGGATAAAGAGA | | UCAGGAUAAAGAGA |
| STMN2_ | + | CTTT | 2056 | TAGTTGTATGTCTTTA | 4062 | UAGUUGUAUGUCUUUA |
| intron1 | | | | TATCAGGATAAAGA | | UAUCAGGAUAAAGA |
| STMN2_ | + | ATTT | 2057 | TTATAGGGCAAAAAT | 4063 | UUAUAGGGCAAAAAUA |
| intron1 | | | | ATTGCCTAGGACTGA | | UUGCCUAGGACUGA |
| STMN2_ | + | TTTA | 2058 | TTTTTATAGGGCAAAA | 4064 | UUUUUAUAGGGCAAAA |
| intron1 | | | | ATATTGCCTAGGAC | | AUAUUGCCUAGGAC |
| STMN2_ | + | ATTT | 2059 | ATTTTTATAGGGCAAA | 4065 | AUUUUUAUAGGGCAAA |
| intron1 | | | | AATATTGCCTAGGA | | AAUAUUGCCUAGGA |
| STMN2_ | + | TTTC | 2060 | AGCCATCATTTTGCT | 4066 | AGCCAUCAUUUUGCUG |
| intron1 | | | | GGTCATGTGGAAATA | | GUCAUGUGGAAAUA |
| STMN2_ | + | ATTT | 2061 | CAGCCATCATTTTGC | 4067 | CAGCCAUCAUUUUGCU |
| intron1 | | | | TGGTCATGTGGAAAT | | GGUCAUGUGGAAAU |
| STMN2_ | + | ATTA | 2062 | ATGCATTTCAGCCATC | 4068 | AUGCAUUUCAGCCAUC |
| intron1 | | | | ATTTTGCTGGTCAT | | AUUUUGCUGGUCAU |
| STMN2_ | + | GTTA | 2063 | ATTAATGCATTTCAG | 4069 | AUUAAUGCAUUUCAG |
| intron1 | | | | CCATCATTTTGCTGG | | CCAUCAUUUUGCUGG |
| STMN2_ | + | TTTA | 2064 | TATGAGTGTAAAGGT | 4070 | UAUGAGUGUAAAGGU |
| intron1 | | | | TAATTAATGCATTTC | | UAAUUAAUGCAUUUC |
| STMN2_ | + | TTTT | 2065 | ATATGAGTGTAAAGG | 4071 | AUAUGAGUGUAAAGGU |
| intron1 | | | | TTAATTAATGCATTT | | UAAUUAAUGCAUUU |
| STMN2_ | + | CTTT | 2066 | TATATGAGTGTAAAG | 4072 | UAUAUGAGUGUAAAGG |
| intron1 | | | | GTTAATTAATGCATT | | UUAAUUAAUGCAUU |
| STMN2_ | + | GTTC | 2067 | TCACAAAACACTTTT | 4073 | UCACAAAACACUUUU |
| intron1 | | | | ATATGAGTGTAAAGG | | AUAUGAGUGUAAAGG |
| STMN2_ | + | TTTG | 2068 | TTCTCACAAAACACT | 4074 | UUCUCACAAAACACU |
| intron1 | | | | TTTATATGAGTGTAA | | UUUAUAUGAGUGUAA |
| STMN2_ | + | ATTT | 2069 | GTTCTCACAAAACACT | 4075 | GUUCUCACAAAACACU |
| intron1 | | | | TTTATATGAGTGTA | | UUUAUAUGAGUGUA |
| STMN2_ | + | TTTG | 2070 | TGTACATTTGTTCTCA | 4076 | UGUACAUUUGUUCUCA |
| intron1 | | | | CAAAACACTTTTAT | | CAAAACACUUUUAU |
| STMN2_ | + | ATTT | 2071 | GTGTACATTTGTTCTC | 4077 | GUGUACAUUUGUUCUC |
| intron1 | | | | ACAAAACACTTTTA | | ACAAAACACUUUUA |
| STMN2_ | + | ATTA | 2072 | AAGATAACATTTGTGT | 4078 | AAGAUAACAUUUGUGU |
| intron1 | | | | ACATTTGTTCTCAC | | ACAUUUGUUCUCAC |
| STMN2_ | + | ATTA | 2073 | GTCATGATTAAAGAT | 4079 | GUCAUGAUUAAAGAUA |
| intron1 | | | | AACATTTGTGTACAT | | ACAUUUGUGUACAU |
| STMN2_ | + | TTTA | 2074 | TTAGTCATGATTAAAG | 4080 | UUAGUCAUGAUUAAAG |
| intron1 | | | | ATAACATTTGTGTA | | AUAACAUUUGUGUA |
| STMN2_ | + | TTTT | 2075 | ATTAGTCATGATTAA | 4081 | AUUAGUCAUGAUUAAA |
| intron1 | | | | AGATAACATTTGTGT | | GAUAACAUUUGUGU |
| STMN2_ | + | TTTT | 2076 | TATTAGTCATGATTA | 4082 | UAUUAGUCAUGAUUAA |
| intron1 | | | | AAGATAACATTTGTG | | AGAUAACAUUUGUG |
| STMN2_ | + | ATTT | 2077 | TTATTAGTCATGATTA | 4083 | UUAUUAGUCAUGAUUA |
| intron1 | | | | AAGATAACATTTGT | | AAGAUAACAUUUGU |
| STMN2_ | + | TTTA | 2078 | TAATATCCATTTTTAT | 4084 | UAAUAUCCAUUUUUAU |
| intron1 | | | | TAGTCATGATTAAA | | UAGUCAUGAUUAAA |
| STMN2_ | + | GTTT | 2079 | ATAATATCCATTTTTA | 4085 | AUAAUAUCCAUUUUUA |
| intron1 | | | | TTAGTCATGATTAA | | UUAGUCAUGAUUAA |
| STMN2_ | + | CTTG | 2080 | TGTTTATAATATCCAT | 4086 | UGUUUAUAAUAUCCAU |
| intron1 | | | | TTTTATTAGTCATG | | UUUUAUUAGUCAUG |
| STMN2_ | + | TTTG | 2081 | CAGTAGTAAAGCTTGT | 4087 | CAGUAGUAAAGCUUGU |
| intron1 | | | | GTTTATAATATCCA | | GUUUAUAAUAUCCA |
| STMN2_ | + | ATTT | 2082 | GCAGTAGTAAAGCTT | 4088 | GCAGUAGUAAAGCUUG |
| intron1 | | | | GTGTTTATAATATCC | | UGUUUAUAAUAUCC |
| STMN2_ | + | TTTG | 2083 | TCAAGGAGACATTTG | 4089 | UCAAGGAGACAUUUGC |
| intron1 | | | | CAGTAGTAAAGCTTG | | AGUAGUAAAGCUUG |
| STMN2_ | + | ATTT | 2084 | GTCAAGGAGACATTT | 4090 | GUCAAGGAGACAUUUG |
| intron1 | | | | GCAGTAGTAAAGCTT | | CAGUAGUAAAGCUU |
| STMN2_ | + | TTTA | 2085 | ATAAAGGAATCAGGC | 4091 | AUAAAGGAAUCAGGCC |
| intron1 | | | | CCTGTCATTTGTCAA | | CUGUCAUUUGUCAA |
| STMN2_ | + | TTTT | 2086 | AATAAAGGAATCAGG | 4092 | AAUAAAGGAAUCAGGC |
| intron1 | | | | CCCTGTCATTTGTCA | | CCUGUCAUUUGUCA |
| STMN2_ | + | ATTT | 2087 | TGCTGGTCATGTGGA | 4093 | UGCUGGUCAUGUGGAA |
| intron1 | | | | AATATAGCTTCTTTA | | AUAUAGCUUCUUUA |
| STMN2_ | + | TTTT | 2088 | GCTGGTCATGTGGAA | 4094 | GCUGGUCAUGUGGAAA |
| intron1 | | | | ATATAGCTTCTTTAG | | UAUAGCUUCUUUAG |
| STMN2_ | + | TTTG | 2089 | CTGGTCATGTGGAAA | 4095 | CUGGUCAUGUGGAAA |
| intron1 | | | | TATAGCTTCTTTAGG | | UAUAGCUUCUUUAGG |
| STMN2_ | + | CTTC | 2090 | TTTAGGAATTGTACT | 4096 | UUUAGGAAUUGUACU |
| intron1 | | | | TAGAGTAGGAGCCAC | | UAGAGUAGGAGCCAC |
| STMN2_ | + | TTTA | 2091 | AAATAATTTATTTTTA | 4097 | AAAUAAUUUAUUUUUA |
| intron1 | | | | TAGGGCAAAAATAT | | UAGGGCAAAAAUAU |
| STMN2_ | + | ATTT | 2092 | AAAATAATTTATTTTT | 4098 | AAAAUAAUUUAUUUUU |
| intron1 | | | | ATAGGGCAAAAATA | | AUAGGGCAAAAAUA |
| STMN2_ | + | GTTC | 2093 | TCATAGAGCACATTTA | 4099 | UCAUAGAGCACAUUUA |
| intron1 | | | | AAATAATTTATTTT | | AAAUAAUUUAUUUU |
| STMN2_ | + | ATTA | 2094 | CAGTTCTCATAGAGCA | 4100 | CAGUUCUCAUAGAGCA |
| intron1 | | | | CATTTAAAATAATT | | CAUUUAAAAUAAUU |
| STMN2_ | + | TTTA | 2095 | TGGCAAGAAATAGAT | 4101 | UGGCAAGAAAUAGAUA |
| intron1 | | | | AATTACAGTTCTCAT | | AUUACAGUUCUCAU |
| STMN2_ | + | TTTT | 2096 | ATGGCAAGAAATAGA | 4102 | AUGGCAAGAAAUAGAU |
| intron1 | | | | TAATTACAGTTCTCA | | AAUUACAGUUCUCA |
| STMN2_ | + | ATTT | 2097 | TATGGCAAGAAATAG | 4103 | UAUGGCAAGAAAUAGA |
| intron1 | | | | ATAATTACAGTTCTC | | UAAUUACAGUUCUC |
| STMN2_ | + | TTTA | 2098 | TTTTATGGCAAGAAAT | 4104 | UUUUAUGGCAAGAAAU |
| intron1 | | | | AGATAATTACAGTT | | AGAUAAUUACAGUU |
| STMN2_ | + | ATTT | 2099 | ATTTTATGGCAAGAA | 4105 | AUUUUAUGGCAAGAAA |
| intron1 | | | | ATAGATAATTACAGT | | UAGAUAAUUACAGU |
| STMN2_ | + | TTTC | 2100 | AAAATTTATTTTATGG | 4106 | AAAAUUUAUUUUAUGG |
| intron1 | | | | CAAGAAATAGATAA | | CAAGAAAUAGAUAA |
| STMN2_ | + | GTTT | 2101 | CAAAATTTATTTTATG | 4107 | CAAAAUUUAUUUUAUG |
| intron1 | | | | GCAAGAAATAGATA | | GCAAGAAAUAGAUA |
| STMN2_ | + | GTTA | 2102 | TGGGTTTCAAAATTTA | 4108 | UGGGUUUCAAAAUUUA |
| intron1 | | | | TTTTATGGCAAGAA | | UUUUAUGGCAAGAA |
| STMN2_ | + | TTTA | 2103 | ATACTCTGGAAAGTTA | 4109 | AUACUCUGGAAAGUUA |
| intron1 | | | | TGGGTTTCAAAATT | | UGGGUUUCAAAAUU |
| STMN2_ | + | CTTA | 2104 | TGAAATGCAGCCATA | 4110 | UGAAAUGCAGCCAUAA |
| intron1 | | | | AAGTTTAACTTCCAT | | AGUUUAACUUCCAU |
| STMN2_ | + | ATTT | 2105 | AATACTCTGGAAAGTT | 4111 | AAUACUCUGGAAAGUU |
| intron1 | | | | ATGGGTTTCAAAAT | | AUGGGUUUCAAAAU |
| STMN2_ | + | GTTG | 2106 | TTGACCTCCAGAGTAA | 4112 | UUGACCUCCAGAGUAA |
| intron1 | | | | AATATTTAATACTC | | AAUAUUUAAUACUC |
| STMN2_ | + | CTTG | 2107 | TTGTTGACCTCCAGAG | 4113 | UUGUUGACCUCCAGAG |
| intron1 | | | | TAAAATATTTAATA | | UAAAAUAUUUAAUA |
| STMN2_ | + | GTTC | 2108 | TCACTTGTTGTTGACC | 4114 | UCACUUGUUGUUGACC |
| intron1 | | | | TCCAGAGTAAAATA | | UCCAGAGUAAAAUA |
| STMN2_ | + | TTTG | 2109 | TTCTCACTTGTTGTT | 4115 | UUCUCACUUGUUGUUG |
| intron1 | | | | GACCTCCAGAGTAAA | | ACCUCCAGAGUAAA |
| STMN2_ | + | GTTT | 2110 | GTTCTCACTTGTTGTT | 4116 | GUUCUCACUUGUUGUU |
| intron1 | | | | GACCTCCAGAGTAA | | GACCUCCAGAGUAA |
| STMN2_ | + | TTTA | 2111 | AGTTTGTTCTCACTT | 4117 | AGUUUGUUCUCACUU |
| intron1 | | | | GTTGTTGACCTCCAG | | GUUGUUGACCUCCAG |
| STMN2_ | + | TTTT | 2112 | AAGTTTGTTCTCACT | 4118 | AAGUUUGUUCUCACU |
| intron1 | | | | TGTTGTTGACCTCCA | | UGUUGUUGACCUCCA |
| STMN2_ | + | ATTT | 2113 | TAAGTTTGTTCTCACT | 4119 | UAAGUUUGUUCUCACU |
| intron1 | | | | TGTTGTTGACCTCC | | UGUUGUUGACCUCC |
| STMN2_ | + | ATTA | 2114 | TACTATAAAACCATA | 4120 | UACUAUAAAACCAUAA |
| intron1 | | | | ACAAAAATATTTTAA | | CAAAAAUAUUUUAA |
| STMN2_ | + | CTTA | 2115 | GAGTAGGAGCCACAT | 4121 | GAGUAGGAGCCACAUA |
| intron1 | | | | ATTATACTATAAAAC | | UUAUACUAUAAAAC |
| STMN2_ | + | ATTG | 2116 | TACTTAGAGTAGGAG | 4122 | UACUUAGAGUAGGAGC |
| intron1 | | | | CCACATATTATACTA | | CACAUAUUAUACUA |
| STMN2_ | + | TTTA | 2117 | GGAATTGTACTTAGA | 4123 | GGAAUUGUACUUAGAG |
| intron1 | | | | GTAGGAGCCACATAT | | UAGGAGCCACAUAU |
| STMN2_ | + | CTTT | 2118 | AGGAATTGTACTTAG | 4124 | AGGAAUUGUACUUAGA |
| intron1 | | | | AGTAGGAGCCACATA | | GUAGGAGCCACAUA |
| STMN2_ | + | GTTG | 2119 | ACCTCCAGAGTAAAA | 4125 | ACCUCCAGAGUAAAAU |
| intron1 | | | | TATTTAATACTCTGG | | AUUUAAUACUCUGG |
| STMN2_ | + | GTTT | 2120 | AACTTCCATTAACAAA | 4126 | AACUUCCAUUAACAAA |
| intron1 | | | | GCTGCTCACAGTAA | | GCUGCUCACAGUAA |
| STMN2_ | + | TTTA | 2121 | ACTTCCATTAACAAAG | 4127 | ACUUCCAUUAACAAAG |
| intron1 | | | | CTGCTCACAGTAAA | | CUGCUCACAGUAAA |
| STMN2_ | + | CTTC | 2122 | CATTAACAAAGCTGCT | 4128 | CAUUAACAAAGCUGCU |
| intron1 | | | | CACAGTAAACCTAT | | CACAGUAAACCUAU |
| STMN2_ | + | ATTT | 2123 | AAAGATTGGTAAATTT | 4129 | AAAGAUUGGUAAAUUU |
| intron1 | | | | AAGCTCAAATAATT | | AAGCUCAAAUAAUU |
| STMN2_ | + | CTTA | 2124 | TTTAAAGATTGGTAAA | 4130 | UUUAAAGAUUGGUAAA |
| intron1 | | | | TTTAAGCTCAAATA | | UUUAAGCUCAAAUA |
| STMN2_ | + | GTTG | 2125 | TCTTATTTAAAGATTG | 4131 | UCUUAUUUAAAGAUUG |
| intron1 | | | | GTAAATTTAAGCTC | | GUAAAUUUAAGCUC |
| STMN2_ | + | CTTC | 2126 | ATATAATCCCTCTGAG | 4132 | AUAUAAUCCCUCUGAG |
| intron1 | | | | ATGGGCATACTATA | | AUGGGCAUACUAUA |
| STMN2_ | + | TTTG | 2127 | AATCTTCATATAATCC | 4133 | AAUCUUCAUAUAAUCC |
| intron1 | | | | CTCTGAGATGGGCA | | CUCUGAGAUGGGCA |
| STMN2_ | + | TTTT | 2128 | GAATCTTCATATAAT | 4134 | GAAUCUUCAUAUAAU |
| intron1 | | | | CCCTCTGAGATGGGC | | CCCUCUGAGAUGGGC |
| STMN2_ | + | CTTT | 2129 | TGAATCTTCATATAA | 4135 | UGAAUCUUCAUAUAA |
| intron1 | | | | TCCCTCTGAGATGGG | | UCCCUCUGAGAUGGG |
| STMN2_ | + | CTTC | 2130 | ATCCTTTTGAATCTTC | 4136 | AUCCUUUUGAAUCUUC |
| intron1 | | | | ATATAATCCCTCTG | | AUAUAAUCCCUCUG |
| STMN2_ | + | ATTC | 2131 | ACCTGCTTCATCCTT | 4137 | ACCUGCUUCAUCCUU |
| intron1 | | | | TTGAATCTTCATATA | | UUGAAUCUUCAUAUA |
| STMN2_ | + | TTTA | 2132 | GAAAACATTCACCTG | 4138 | GAAAACAUUCACCUG |
| intron1 | | | | CTTCATCCTTTTGAA | | CUUCAUCCUUUUGAA |
| STMN2_ | + | TTTT | 2133 | AGAAAACATTCACCT | 4139 | AGAAAACAUUCACCU |
| intron1 | | | | GCTTCATCCTTTTGA | | GCUUCAUCCUUUUGA |
| STMN2_ | + | TTTT | 2134 | TAGAAAACATTCACC | 4140 | UAGAAAACAUUCACC |
| intron1 | | | | TGCTTCATCCTTTTG | | UGCUUCAUCCUUUUG |
| STMN2_ | + | ATTT | 2135 | TTAGAAAACATTCAC | 4141 | UUAGAAAACAUUCAC |
| intron1 | | | | CTGCTTCATCCTTTT | | CUGCUUCAUCCUUUU |
| STMN2_ | + | CTTG | 2136 | TCATTTTTAGAAAAC | 4142 | UCAUUUUUAGAAAAC |
| intron1 | | | | ATTCACCTGCTTCAT | | AUUCACCUGCUUCAU |
| STMN2_ | + | ATTA | 2137 | AATCGCATGATCTAT | 4143 | AAUCGCAUGAUCUAU |
| intron1 | | | | CTATATGGGACCTTG | | CUAUAUGGGACCUUG |
| STMN2_ | + | GTTC | 2138 | AAAAGAAAAATTAAA | 4144 | AAAAGAAAAAUUAAA |
| intron1 | | | | TCGCATGATCTATCT | | UCGCAUGAUCUAUCU |
| STMN2_ | + | TTTA | 2139 | AAAGGAGCAGGCAAG | 4145 | AAAGGAGCAGGCAAG |
| intron1 | | | | CATAGAAGACTAAAA | | CAUAGAAGACUAAAA |
| STMN2_ | + | TTTT | 2140 | AAAAGGAGCAGGCAA | 4146 | AAAAGGAGCAGGCAA |
| intron1 | | | | GCATAGAAGACTAAA | | GCAUAGAAGACUAAA |
| STMN2_ | + | TTTT | 2141 | TAAAAGGAGCAGGCA | 4147 | UAAAAGGAGCAGGCA |
| intron1 | | | | AGCATAGAAGACTAA | | AGCAUAGAAGACUAA |
| STMN2_ | + | TTTT | 2142 | TTAAAAGGAGCAGGC | 4148 | UUAAAAGGAGCAGGC |
| intron1 | | | | AAGCATAGAAGACTA | | AAGCAUAGAAGACUA |
| STMN2_ | + | GTTT | 2143 | TTTAAAAGGAGCAGG | 4149 | UUUAAAAGGAGCAGG |
| intron1 | | | | CAAGCATAGAAGACT | | CAAGCAUAGAAGACU |
| STMN2_ | + | CTTA | 2144 | TATAGTTTTTTAAAA | 4150 | UAUAGUUUUUUAAAA |
| intron1 | | | | GGAGCAGGCAAGCAT | | GGAGCAGGCAAGCAU |
| STMN2_ | + | TTTC | 2145 | TTATATAGTTTTTTA | 4151 | UUAUAUAGUUUUUUA |
| intron1 | | | | AAAGGAGCAGGCAAG | | AAAGGAGCAGGCAAG |
| STMN2_ | + | TTTT | 2146 | CTTATATAGTTTTTT | 4152 | CUUAUAUAGUUUUUU |
| intron1 | | | | AAAAGGAGCAGGCAA | | AAAAGGAGCAGGCAA |
| STMN2_ | + | TTTT | 2147 | TCTTATATAGTTTTT | 4153 | UCUUAUAUAGUUUUU |
| intron1 | | | | TAAAAGGAGCAGGCA | | UAAAAGGAGCAGGCA |
| STMN2_ | + | TTTT | 2148 | TTCTTATATAGTTTT | 4154 | UUCUUAUAUAGUUUU |
| intron1 | | | | TTAAAAGGAGCAGGC | | UUAAAAGGAGCAGGC |
| STMN2_ | + | TTTT | 2149 | TTTCTTATATAGTTT | 4155 | UUUCUUAUAUAGUUU |
| intron1 | | | | TTTAAAAGGAGCAGG | | UUUAAAAGGAGCAGG |
| STMN2_ | + | TTTA | 2150 | AAGATTGGTAAATTT | 4156 | AAGAUUGGUAAAUUU |
| intron1 | | | | AAGCTCAAATAATTT | | AAGCUCAAAUAAUUU |
| STMN2_ | + | ATTG | 2151 | GTAAATTTAAGCTCA | 4157 | GUAAAUUUAAGCUCA |
| intron1 | | | | AATAATTTATTCAGT | | AAUAAUUUAUUCAGU |
| STMN2_ | + | ATTT | 2152 | AAGCTCAAATAATTT | 4158 | AAGCUCAAAUAAUUU |
| intron1 | | | | ATTCAGTGGCAAGCC | | AUUCAGUGGCAAGCC |
| STMN2_ | + | TTTA | 2153 | AGCTCAAATAATTTA | 4159 | AGCUCAAAUAAUUUA |
| intron1 | | | | TTCAGTGGCAAGCCT | | UUCAGUGGCAAGCCU |
| STMN2_ | + | TTTG | 2154 | TTCTGAAGCCTGTGC | 4160 | UUCUGAAGCCUGUGC |
| intron1 | | | | CAGGTATTATGAGAA | | CAGGUAUUAUGAGAA |
| STMN2_ | + | TTTT | 2155 | GATTACATTTTATGT | 4161 | GAUUACAUUUUAUGU |
| intron1 | | | | AATTCTAATCCAGCT | | AAUUCUAAUCCAGCU |
| STMN2_ | + | CTTT | 2156 | GTTCTGAAGCCTGTG | 4162 | GUUCUGAAGCCUGUG |
| intron1 | | | | CCAGGTATTATGAGA | | CCAGGUAUUAUGAGA |
| STMN2_ | + | ATTG | 2157 | GAGCACCAACTTTGT | 4163 | GAGCACCAACUUUGU |
| intron1 | | | | TCTGAAGCCTGTGCC | | UCUGAAGCCUGUGCC |
| STMN2_ | + | ATTG | 2158 | ATAGTCAGTGTCACT | 4164 | AUAGUCAGUGUCACU |
| intron1 | | | | AACTAAAGTAAAATA | | AACUAAAGUAAAAUA |
| STMN2_ | + | TTTA | 2159 | AAGTCATTGATAGTC | 4165 | AAGUCAUUGAUAGUC |
| intron1 | | | | AGTGTCACTAACTAA | | AGUGUCACUAACUAA |
| STMN2_ | + | GTTT | 2160 | AAAGTCATTGATAGT | 4166 | AAAGUCAUUGAUAGU |
| intron1 | | | | CAGTGTCACTAACTA | | CAGUGUCACUAACUA |
| STMN2_ | + | CTTC | 2161 | AGTTTAAAGTCATTG | 4167 | AGUUUAAAGUCAUUG |
| intron1 | | | | ATAGTCAGTGTCACT | | AUAGUCAGUGUCACU |
| STMN2_ | + | TTTG | 2162 | TCTTCAGTTTAAAGT | 4168 | UCUUCAGUUUAAAGU |
| intron1 | | | | CATTGATAGTCAGTG | | CAUUGAUAGUCAGUG |
| STMN2_ | + | ATTT | 2163 | GTCTTCAGTTTAAAG | 4169 | GUCUUCAGUUUAAAG |
| intron1 | | | | TCATTGATAGTCAGT | | UCAUUGAUAGUCAGU |
| STMN2_ | + | TTTA | 2164 | GCACTCCCTCCACTG | 4170 | GCACUCCCUCCACUG |
| intron1 | | | | TCCTGTAATAAAACA | | UCCUGUAAUAAAACA |
| STMN2_ | + | GTTT | 2165 | AGCACTCCCTCCACT | 4171 | AGCACUCCCUCCACU |
| intron1 | | | | GTCCTGTAATAAAAC | | GUCCUGUAAUAAAAC |
| STMN2_ | + | ATTC | 2166 | ATGCAAAATAAGGTT | 4172 | AUGCAAAAUAAGGUU |
| intron1 | | | | TAGCACTCCCTCCAC | | UAGCACUCCCUCCAC |
| STMN2_ | + | ATTT | 2167 | TTTTCTTATATAGTT | 4173 | UUUUCUUAUAUAGUU |
| intron1 | | | | TTTTAAAAGGAGCAG | | UUUUAAAAGGAGCAG |
| STMN2_ | + | TTTC | 2168 | ATACATATATACACA | 4174 | AUACAUAUAUACACA |
| intron1 | | | | TTCATGCAAAATAAG | | UUCAUGCAAAAUAAG |
| STMN2_ | + | GTTA | 2169 | TATATCATGTATGTG | 4175 | UAUAUCAUGUAUGUG |
| intron1 | | | | CCTATTTCATACATA | | CCUAUUUCAUACAUA |
| STMN2_ | + | TTTA | 2170 | TATGTAATATATAAA | 4176 | UAUGUAAUAUAUAAA |
| intron1 | | | | TATGTTATATATCAT | | UAUGUUAUAUAUCAU |
| STMN2_ | + | ATTT | 2171 | ATATGTAATATATAA | 4177 | AUAUGUAAUAUAUAA |
| intron1 | | | | ATATGTTATATATCA | | AUAUGUUAUAUAUCA |
| STMN2_ | + | TTTA | 2172 | CCTATCAAAATATTT | 4178 | CCUAUCAAAAUAUUU |
| intron1 | | | | ATATGTAATATATAA | | AUAUGUAAUAUAUAA |
| STMN2_ | + | ATTT | 2173 | ACCTATCAAAATATT | 4179 | ACCUAUCAAAAUAUU |
| intron1 | | | | TATATGTAATATATA | | UAUAUGUAAUAUAUA |
| STMN2_ | + | ATTA | 2174 | TTTACCTATCAAAAT | 4180 | UUUACCUAUCAAAAU |
| intron1 | | | | ATTTATATGTAATAT | | AUUUAUAUGUAAUAU |
| STMN2_ | + | GTTG | 2175 | TGTATATTATTTACC | 4181 | UGUAUAUUAUUUACC |
| intron1 | | | | TATCAAAATATTTAT | | UAUCAAAAUAUUUAU |
| STMN2_ | + | ATTA | 2176 | CATATAATAAAGTTG | 4182 | CAUAUAAUAAAGUUG |
| intron1 | | | | TGTATATTATTTACC | | UGUAUAUUAUUUACC |
| STMN2_ | + | ATTA | 2177 | TAACATATAATATAT | 4183 | UAACAUAUAAUAUAU |
| intron1 | | | | ATATTACATATAATA | | AUAUUACAUAUAAUA |
| STMN2_ | + | ATTA | 2178 | TATATATATTATAAC | 4184 | UAUAUAUAUUAUAAC |
| intron1 | | | | ATATAATATATATAT | | AUAUAAUAUAUAUAU |
| STMN2_ | + | ATTC | 2179 | AGTGGCAAGCCTCAG | 4185 | AGUGGCAAGCCUCAG |
| intron1 | | | | AGGCAGACTCGGAAC | | AGGCAGACUCGGAAC |
| STMN2_ | + | TTTA | 2180 | TTCAGTGGCAAGCCT | 4186 | UUCAGUGGCAAGCCU |
| intron1 | | | | CAGAGGCAGACTCGG | | CAGAGGCAGACUCGG |
| STMN2_ | + | ATTT | 2181 | ATTCAGTGGCAAGCC | 4187 | AUUCAGUGGCAAGCC |
| intron1 | | | | TCAGAGGCAGACTCG | | UCAGAGGCAGACUCG |
| STMN2_ | + | ATTT | 2182 | CATACATATATACAC | 4188 | CAUACAUAUAUACAC |
| intron1 | | | | ATTCATGCAAAATAA | | AUUCAUGCAAAAUAA |
| STMN2_ | + | CTTT | 2183 | TAATAAAGGAATCAG | 4189 | UAAUAAAGGAAUCAG |
| intron1 | | | | GCCCTGTCATTTGTC | | GCCCUGUCAUUUGUC |
| STMN2_ | + | TTTC | 2184 | TGATGATTTTTTTCT | 4190 | UGAUGAUUUUUUUCU |
| intron1 | | | | TATATAGTTTTTTAA | | UAUAUAGUUUUUUAA |
| STMN2_ | + | ATTA | 2185 | TATTTCTGATGATTT | 4191 | UAUUUCUGAUGAUUU |
| intron1 | | | | TTTTCTTATATAGTT | | UUUUCUUAUAUAGUU |
| STMN2_ | + | CTTT | 2186 | TTATTTCCAACAAAA | 4192 | UUAUUUCCAACAAAA |
| intron1 | | | | ATATCTATTGTTATT | | AUAUCUAUUGUUAUU |
| STMN2_ | + | GTTA | 2187 | CTTTTTATTTCCAAC | 4193 | CUUUUUAUUUCCAAC |
| intron1 | | | | AAAAATATCTATTGT | | AAAAAUAUCUAUUGU |
| STMN2_ | + | ATTA | 2188 | ATGCAGAGTTACTTT | 4194 | AUGCAGAGUUACUUU |
| intron1 | | | | TTATTTCCAACAAAA | | UUAUUUCCAACAAAA |
| STMN2_ | + | TTTA | 2189 | TTAATGCAGAGTTAC | 4195 | UUAAUGCAGAGUUAC |
| intron1 | | | | TTTTTATTTCCAACA | | UUUUUAUUUCCAACA |
| STMN2_ | + | TTTT | 2190 | ATTAATGCAGAGTTA | 4196 | AUUAAUGCAGAGUUA |
| intron1 | | | | CTTTTTATTTCCAAC | | CUUUUUAUUUCCAAC |
| STMN2_ | + | TTTT | 2191 | TATTAATGCAGAGTT | 4197 | UAUUAAUGCAGAGUU |
| intron1 | | | | ACTTTTTATTTCCAA | | ACUUUUUAUUUCCAA |
| STMN2_ | + | ATTT | 2192 | TTATTAATGCAGAGT | 4198 | UUAUUAAUGCAGAGU |
| intron1 | | | | TACTTTTTATTTCCA | | UACUUUUUAUUUCCA |
| STMN2_ | + | ATTA | 2193 | TTTTTATTAATGCAG | 4199 | UUUUUAUUAAUGCAG |
| intron1 | | | | AGTTACTTTTTATTT | | AGUUACUUUUUAUUU |
| STMN2_ | + | CTTC | 2194 | AGAACATAATTATTT | 4200 | AGAACAUAAUUAUUU |
| intron1 | | | | TTATTAATGCAGAGT | | UUAUUAAUGCAGAGU |
| STMN2_ | + | ATTG | 2195 | CAGCCTCCCTGGGAA | 4201 | CAGCCUCCCUGGGAA |
| intron1 | | | | CTCTGCTTCAGAACA | | CUCUGCUUCAGAACA |
| STMN2_ | + | CTTA | 2196 | TTGCAGCCTCCCTGG | 4202 | UUGCAGCCUCCCUGG |
| intron1 | | | | GAACTCTGCTTCAGA | | GAACUCUGCUUCAGA |
| STMN2_ | + | TTTA | 2197 | GGATAGACTTATTGC | 4203 | GGAUAGACUUAUUGC |
| intron1 | | | | AGCCTCCCTGGGAAC | | AGCCUCCCUGGGAAC |
| STMN2_ | + | TTTT | 2198 | AGGATAGACTTATTG | 4204 | AGGAUAGACUUAUUG |
| intron1 | | | | CAGCCTCCCTGGGAA | | CAGCCUCCCUGGGAA |
| STMN2_ | + | CTTT | 2199 | TAGGATAGACTTATT | 4205 | UAGGAUAGACUUAUU |
| intron1 | | | | GCAGCCTCCCTGGGA | | GCAGCCUCCCUGGGA |
| STMN2_ | + | ATTA | 2200 | ATCATCTCAGGCACT | 4206 | AUCAUCUCAGGCACU |
| intron1 | | | | TTTAGGATAGACTTA | | UUUAGGAUAGACUUA |
| STMN2_ | + | ATTT | 2201 | CCAGACTCTCGGGAA | 4207 | CCAGACUCUCGGGAA |
| intron1 | | | | GAACATTAATCATCT | | GAACAUUAAUCAUCU |
| STMN2_ | + | GTTA | 2202 | TCATTTCCAGACTCT | 4208 | UCAUUUCCAGACUCU |
| intron1 | | | | CGGGAAGAACATTAA | | CGGGAAGAACAUUAA |
| STMN2_ | + | GTTA | 2203 | CAAAACTGAGACCAG | 4209 | CAAAACUGAGACCAG |
| intron1 | | | | AAAATCCCATCAAGA | | AAAAUCCCAUCAAGA |
| STMN2_ | + | ATTG | 2204 | ACTGTTACAAAACTG | 4210 | ACUGUUACAAAACUG |
| intron1 | | | | AGACCAGAAAATCCC | | AGACCAGAAAAUCCC |
| STMN2_ | + | CTTA | 2205 | TAATATATTGACTGT | 4211 | UAAUAUAUUGACUGU |
| intron1 | | | | TACAAAACTGAGACC | | UACAAAACUGAGACC |
| STMN2_ | + | CTTC | 2206 | CTAGTGAGGAGCAAC | 4212 | CUAGUGAGGAGCAAC |
| intron1 | | | | CTAACTCACACGAAA | | CUAACUCACACGAAA |
| STMN2_ | + | TTTG | 2207 | GGCTTCCTAGTGAGG | 4213 | GGCUUCCUAGUGAGG |
| intron1 | | | | AGCAACCTAACTCAC | | AGCAACCUAACUCAC |
| STMN2_ | + | GTTT | 2208 | GGGCTTCCTAGTGAG | 4214 | GGGCUUCCUAGUGAG |
| intron1 | | | | GAGCAACCTAACTCA | | GAGCAACCUAACUCA |
| STMN2_ | + | TTTC | 2209 | CCAGTTTGGGCTTCC | 4215 | CCAGUUUGGGCUUCC |
| intron1 | | | | TAGTGAGGAGCAACC | | UAGUGAGGAGCAACC |
| STMN2_ | + | GTTT | 2210 | CCCAGTTTGGGCTTC | 4216 | CCCAGUUUGGGCUUC |
| intron1 | | | | CTAGTGAGGAGCAAC | | CUAGUGAGGAGCAAC |
| STMN2_ | + | ATTA | 2211 | TAATAATAGTTTCCC | 4217 | UAAUAAUAGUUUCCC |
| intron1 | | | | AGTTTGGGCTTCCTA | | AGUUUGGGCUUCCUA |
| STMN2_ | + | ATTA | 2212 | ACAAAGCTGCTCACA | 4218 | ACAAAGCUGCUCACA |
| intron1 | | | | GTAAACCTATTATAA | | GUAAACCUAUUAUAA |
| STMN2_ | + | TTTT | 2213 | TATTTCCAACAAAAA | 4219 | UAUUUCCAACAAAAA |
| intron1 | | | | TATCTATTGTTATTA | | UAUCUAUUGUUAUUA |
| STMN2_ | + | TTTT | 2214 | ATTTCCAACAAAAAT | 4220 | AUUUCCAACAAAAAU |
| intron1 | | | | ATCTATTGTTATTAT | | AUCUAUUGUUAUUAU |
| STMN2_ | + | TTTA | 2215 | TTTCCAACAAAAATA | 4221 | UUUCCAACAAAAAUA |
| intron1 | | | | TCTATTGTTATTATT | | UCUAUUGUUAUUAUU |
| STMN2_ | + | ATTT | 2216 | CCAACAAAAATATCT | 4222 | CCAACAAAAAUAUCU |
| intron1 | | | | ATTGTTATTATTTAA | | AUUGUUAUUAUUUAA |
| STMN2_ | + | TTTA | 2217 | TTATATTTCTGATGA | 4223 | UUAUAUUUCUGAUGA |
| intron1 | | | | TTTTTTTCTTATATA | | UUUUUUUCUUAUAUA |
| STMN2_ | + | TTTT | 2218 | ATTATATTTCTGATG | 4224 | AUUAUAUUUCUGAUG |
| intron1 | | | | ATTTTTTTCTTATAT | | AUUUUUUUCUUAUAU |
| STMN2_ | + | TTTT | 2219 | TATTATATTTCTGAT | 4225 | UAUUAUAUUUCUGAU |
| intron1 | | | | GATTTTTTTCTTATA | | GAUUUUUUUCUUAUA |
| STMN2_ | + | CTTT | 2220 | TTATTATATTTCTGA | 4226 | UUAUUAUAUUUCUGA |
| intron1 | | | | TGATTTTTTTCTTAT | | UGAUUUUUUUCUUAU |
| STMN2_ | + | ATTA | 2221 | TCTTTTTATTATATT | 4227 | UCUUUUUAUUAUAUU |
| intron1 | | | | TCTGATGATTTTTTT | | UCUGAUGAUUUUUUU |
| STMN2_ | + | TTTA | 2222 | AAAATTATCTTTTTA | 4228 | AAAAUUAUCUUUUUA |
| intron1 | | | | TTATATTTCTGATGA | | UUAUAUUUCUGAUGA |
| STMN2_ | + | CTTT | 2223 | AAAAATTATCTTTTT | 4229 | AAAAAUUAUCUUUUU |
| intron1 | | | | ATTATATTTCTGATG | | AUUAUAUUUCUGAUG |
| STMN2_ | + | CTTG | 2224 | TCACTTTAAAAATTA | 4230 | UCACUUUAAAAAUUA |
| intron1 | | | | TCTTTTTATTATATT | | UCUUUUUAUUAUAUU |
| STMN2_ | + | ATTA | 2225 | CATGATCCTGCACTC | 4231 | CAUGAUCCUGCACUC |
| intron1 | | | | TTGTCACTTTAAAAA | | UUGUCACUUUAAAAA |
| STMN2_ | + | TTTA | 2226 | ATGACATATTACATG | 4232 | AUGACAUAUUACAUG |
| intron1 | | | | ATCCTGCACTCTTGT | | AUCCUGCACUCUUGU |
| STMN2_ | + | TTTT | 2227 | AATGACATATTACAT | 4233 | AAUGACAUAUUACAU |
| intron1 | | | | GATCCTGCACTCTTG | | GAUCCUGCACUCUUG |
| STMN2_ | 1 | CTTT | 2228 | TAATGACATATTACA | 4234 | UAAUGACAUAUUACA |
| intron1 | | | | TGATCCTGCACTCTT | | UGAUCCUGCACUCUU |
| STMN2_ | + | GTTC | 2229 | TAGTCTTTTAATGAC | 4235 | UAGUCUUUUAAUGAC |
| intron1 | | | | ATATTACATGATCCT | | AUAUUACAUGAUCCU |
| STMN2_ | + | ATTT | 2230 | CTGATGATTTTTTTC | 4236 | CUGAUGAUUUUUUUC |
| intron1 | | | | TTATATAGTTTTTTA | | UUAUAUAGUUUUUUA |
| STMN2_ | + | GTTG | 2231 | TTCTAGTCTTTTAAT | 4237 | UUCUAGUCUUUUAAU |
| intron1 | | | | GACATATTACATGAT | | GACAUAUUACAUGAU |
| STMN2_ | + | ATTC | 2232 | AAACACATGAAAAAT | 4238 | AAACACAUGAAAAAU |
| intron1 | | | | TACCAAAGTTGTTCT | | UACCAAAGUUGUUCU |
| STMN2_ | 1 | CTTC | 2233 | TCATAATAAATATTC | 4239 | UCAUAAUAAAUAUUC |
| intron1 | | | | AAACACATGAAAAAT | | AAACACAUGAAAAAU |
| STMN2_ | + | ATTA | 2234 | GCACCCTTCTCATAA | 4240 | GCACCCUUCUCAUAA |
| intron1 | | | | TAAATATTCAAACAC | | UAAAUAUUCAAACAC |
| STMN2_ | + | ATTC | 2235 | CAATTAGCACCCTTC | 4241 | CAAUUAGCACCCUUC |
| intron1 | | | | TCATAATAAATATTC | | UCAUAAUAAAUAUUC |
| STMN2_ | + | TTTA | 2236 | TCTGAGAAATTCCAA | 4242 | UCUGAGAAAUUCCAA |
| intron1 | | | | TTAGCACCCTTCTCA | | UUAGCACCCUUCUCA |
| STMN2_ | + | CTTT | 2237 | ATCTGAGAAATTCCA | 4243 | AUCUGAGAAAUUCCA |
| intron1 | | | | ATTAGCACCCTTCTC | | AUUAGCACCCUUCUC |
| STMN2_ | + | CTTA | 2238 | CAGCTTTATCTGAGA | 4244 | CAGCUUUAUCUGAGA |
| intron1 | | | | AATTCCAATTAGCAC | | AAUUCCAAUUAGCAC |
| STMN2_ | + | TTTA | 2239 | AGTCTTACAGCTTTA | 4245 | AGUCUUACAGCUUUA |
| intron1 | | | | TCTGAGAAATTCCAA | | UCUGAGAAAUUCCAA |
| STMN2_ | + | ATTT | 2240 | AAGTCTTACAGCTTT | 4246 | AAGUCUUACAGCUUU |
| intron1 | | | | ATCTGAGAAATTCCA | | AUCUGAGAAAUUCCA |
| STMN2_ | + | ATTA | 2241 | TTTAAGTCTTACAGC | 4247 | UUUAAGUCUUACAGC |
| intron1 | | | | TTTATCTGAGAAATT | | UUUAUCUGAGAAAUU |
| STMN2_ | + | GTTA | 2242 | TTATTTAAGTCTTAC | 4248 | UUAUUUAAGUCUUAC |
| intron1 | | | | AGCTTTATCTGAGAA | | AGCUUUAUCUGAGAA |
| STMN2_ | + | ATTG | 2243 | TTATTATTTAAGTCT | 4249 | UUAUUAUUUAAGUCU |
| intron1 | | | | TACAGCTTTATCTGA | | UACAGCUUUAUCUGA |
| STMN2_ | + | TTTC | 2244 | CAACAAAAATATCTA | 4250 | CAACAAAAAUAUCUA |
| intron1 | | | | TTGTTATTATTTAAG | | UUGUUAUUAUUUAAG |
| STMN2_ | + | ATTA | 2245 | CCAAAGTTGTTCTAG | 4251 | CCAAAGUUGUUCUAG |
| intron1 | | | | TCTTTTAATGACATA | | UCUUUUAAUGACAUA |
| STMN2_ | + | ATTA | 2246 | TCTTTTAATAAAGGA | 4252 | UCUUUUAAUAAAGGA |
| intron1 | | | | ATCAGGCCCTGTCAT | | AUCAGGCCCUGUCAU |
| STMN2_ | + | TTTC | 2247 | CAGACTCTCGGGAAG | 4253 | CAGACUCUCGGGAAG |
| intron1 | | | | AACATTAATCATCTC | | AACAUUAAUCAUCUC |
| STMN2_ | + | TTTT | 2248 | AATTATCTTTTAATA | 4254 | AAUUAUCUUUUAAUA |
| intron1 | | | | AAGGAATCAGGCCCT | | AAGGAAUCAGGCCCU |
| STMN2_ | + | CTTC | 2249 | ATTATTCAATTCTAA | 4255 | AUUAUUCAAUUCUAA |
| intron1 | | | | CTTTCTAAGGAAGTC | | CUUUCUAAGGAAGUC |
| STMN2_ | + | CTTA | 2250 | TCTAAGCCAATAAAG | 4256 | UCUAAGCCAAUAAAG |
| intron1 | | | | GATCTTCATTATTCA | | GAUCUUCAUUAUUCA |
| STMN2_ | + | CTTC | 2251 | TGCTTATCTAAGCCA | 4257 | UGCUUAUCUAAGCCA |
| intron1 | | | | ATAAAGGATCTTCAT | | AUAAAGGAUCUUCAU |
| STMN2_ | + | TTTC | 2252 | TTCTGCTTATCTAAG | 4258 | UUCUGCUUAUCUAAG |
| intron1 | | | | CCAATAAAGGATCTT | | CCAAUAAAGGAUCUU |
| STMN2_ | + | TTTT | 2253 | CTTCTGCTTATCTAA | 4259 | CUUCUGCUUAUCUAA |
| intron1 | | | | GCCAATAAAGGATCT | | GCCAAUAAAGGAUCU |
| STMN2_ | + | GTTT | 2254 | TCTTCTGCTTATCTA | 4260 | UCUUCUGCUUAUCUA |
| intron1 | | | | AGCCAATAAAGGATC | | AGCCAAUAAAGGAUC |
| STMN2_ | + | TTTG | 2255 | AAAAGAGTGTTTTCT | 4261 | AAAAGAGUGUUUUCU |
| | | | | TCTGCTTATCTAAGC | | UCUGCUUAUCUAAGC |
| intron1 | | | | | | |
| STMN2_ | + | ATTT | 2256 | GAAAAGAGTGTTTTC | 4262 | GAAAAGAGUGUUUUC |
| | | | | TTCTGCTTATCTAAG | | UUCUGCUUAUCUAAG |
| intron1 | | | | | | |
| STMN2_ | + | ATTG | 2257 | AGTATGACTGTATAT | 4263 | AGUAUGACUGUAUAU |
| | | | | TTGAAAAGAGTGTTT | | UUGAAAAGAGUGUUU |
| intron1 | | | | | | |
| STMN2_ | + | TTTA | 2258 | TTGAGTATGACTGTA | 4264 | UUGAGUAUGACUGUA |
| intron1 | | | | TATTTGAAAAGAGTG | | UAUUUGAAAAGAGUG |
| STMN2_ | + | ATTT | 2259 | ATTGAGTATGACTGT | 4265 | AUUGAGUAUGACUGU |
| intron1 | | | | ATATTTGAAAAGAGT | | AUAUUUGAAAAGAGU |
| STMN2_ | + | CTTA | 2260 | AGAATTTATTGAGTA | 4266 | AGAAUUUAUUGAGUA |
| intron1 | | | | TGACTGTATATTTGA | | UGACUGUAUAUUUGA |
| STMN2_ | + | ATTC | 2261 | TTAAGAATTTATTGA | 4267 | UUAAGAAUUUAUUGA |
| intron1 | | | | GTATGACTGTATATT | | GUAUGACUGUAUAUU |
| STMN2_ | + | CTTC | 2262 | CTGAATACCATGTGA | 4268 | CUGAAUACCAUGUGA |
| intron1 | | | | GAAAATTCTTAAGAA | | GAAAAUUCUUAAGAA |
| STMN2_ | + | TTTC | 2263 | TTCCTGAATACCATG | 4269 | UUCCUGAAUACCAUG |
| intron1 | | | | TGAGAAAATTCTTAA | | UGAGAAAAUUCUUAA |
| STMN2_ | + | ATTT | 2264 | CTTCCTGAATACCAT | 4270 | CUUCCUGAAUACCAU |
| intron1 | | | | GTGAGAAAATTCTTA | | GUGAGAAAAUUCUUA |
| STMN2_ | + | ATTC | 2265 | TAAGAGTATTTCTTC | 4271 | UAAGAGUAUUUCUUC |
| intron1 | | | | CTGAATACCATGTGA | | CUGAAUACCAUGUGA |
| STMN2_ | + | ATTA | 2266 | TTCTAAGAGTATTTC | 4272 | UUCUAAGAGUAUUUC |
| intron1 | | | | TTCCTGAATACCATG | | UUCCUGAAUACCAUG |
| STMN2_ | + | TTTA | 2267 | CCAAATTATTCTAAG | 4273 | CCAAAUUAUUCUAAG |
| intron1 | | | | AGTATTTCTTCCTGA | | AGUAUUUCUUCCUGA |
| STMN2_ | + | ATTT | 2268 | ACCAAATTATTCTAA | 4274 | ACCAAAUUAUUCUAA |
| intron1 | | | | GAGTATTTCTTCCTG | | GAGUAUUUCUUCCUG |
| STMN2_ | + | ATTA | 2269 | TTTACCAAATTATTC | 4275 | UUUACCAAAUUAUUC |
| intron1 | | | | TAAGAGTATTTCTTC | | UAAGAGUAUUUCUUC |
| STMN2_ | 1 | TTTA | 2270 | TTATTTACCAAATTA | 4276 | UUAUUUACCAAAUUA |
| intron1 | | | | TTCTAAGAGTATTTC | | UUCUAAGAGUAUUUC |
| STMN2_ | + | ATTT | 2271 | ATTATTTACCAAATT | 4277 | AUUAUUUACCAAAUU |
| intron1 | | | | ATTCTAAGAGTATTT | | AUUCUAAGAGUAUUU |
| STMN2_ | + | CTTA | 2272 | TATTTATTATTTACC | 4278 | UAUUUAUUAUUUACC |
| intron1 | | | | AAATTATTCTAAGAG | | AAAUUAUUCUAAGAG |
| STMN2_ | + | ATTG | 2273 | CTGTCTCAATATATC | 4279 | CUGUCUCAAUAUAUC |
| intron1 | | | | TTATATTTATTATTT | | UUAUAUUUAUUAUUU |
| STMN2_ | + | ATTA | 2274 | AAACAAAAGATTGCT | 4280 | AAACAAAAGAUUGCU |
| intron1 | | | | GTCTCAATATATCTT | | GUCUCAAUAUAUCUU |
| STMN2_ | + | TTTA | 2275 | TGAATAGCAATACTG | 4281 | UGAAUAGCAAUACUG |
| intron1 | | | | AAGAAATTAAAACAA | | AAGAAAUUAAAACAA |
| STMN2_ | + | ATTA | 2276 | TTCAATTCTAACTTT | 4282 | UUCAAUUCUAACUUU |
| intron1 | | | | CTAAGGAAGTCAACC | | CUAAGGAAGUCAACC |
| STMN2_ | + | ATTC | 2277 | AATTCTAACTTTCTA | 4283 | AAUUCUAACUUUCUA |
| intron1 | | | | AGGAAGTCAACCTAC | | AGGAAGUCAACCUAC |
| STMN2_ | + | ATTC | 2278 | TAACTTTCTAAGGAA | 4284 | UAACUUUCUAAGGAA |
| intron1 | | | | GTCAACCTACAGATC | | GUCAACCUACAGAUC |
| STMN2_ | + | CTTT | 2279 | CTAAGGAAGTCAACC | 4285 | CUAAGGAAGUCAACC |
| intron1 | | | | TACAGATCAGAAAGA | | UACAGAUCAGAAAGA |
| STMN2_ | + | TTTG | 2280 | CAATTTCTTGTACAT | 4286 | CAAUUUCUUGUACAU |
| intron1 | | | | TGAAGGAAAGGAAGA | | UGAAGGAAAGGAAGA |
| STMN2_ | + | TTTT | 2281 | GCAATTTCTTGTACA | 4287 | GCAAUUUCUUGUACA |
| intron1 | | | | TTGAAGGAAAGGAAG | | UUGAAGGAAAGGAAG |
| STMN2_ | + | TTTT | 2282 | TGCAATTTCTTGTAC | 4288 | UGCAAUUUCUUGUAC |
| intron1 | | | | ATTGAAGGAAAGGAA | | AUUGAAGGAAAGGAA |
| STMN2_ | + | ATTT | 2283 | TTGCAATTTCTTGTA | 4289 | UUGCAAUUUCUUGUA |
| intron1 | | | | CATTGAAGGAAAGGA | | CAUUGAAGGAAAGGA |
| STMN2_ | + | TTTC | 2284 | CATTTTTGCAATTTC | 4290 | CAUUUUUGCAAUUUC |
| intron1 | | | | TTGTACATTGAAGGA | | UUGUACAUUGAAGGA |
| STMN2_ | + | CTTT | 2285 | CCATTTTTGCAATTT | 4291 | CCAUUUUUGCAAUUU |
| intron1 | | | | CTTGTACATTGAAGG | | CUUGUACAUUGAAGG |
| STMN2_ | + | TTTC | 2286 | AGGGTCTCTCAGAAG | 4292 | AGGGUCUCUCAGAAG |
| intron1 | | | | CTGGGAAACTTTCCA | | CUGGGAAACUUUCCA |
| STMN2_ | + | ATTT | 2287 | CAGGGTCTCTCAGAA | 4293 | CAGGGUCUCUCAGAA |
| intron1 | | | | GCTGGGAAACTTTCC | | GCUGGGAAACUUUCC |
| STMN2_ | + | GTTC | 2288 | ATTTCAGGGTCTCTC | 4294 | AUUUCAGGGUCUCUC |
| intron1 | | | | AGAAGCTGGGAAACT | | AGAAGCUGGGAAACU |
| STMN2_ | + | GTTA | 2289 | ACAGTTCATTTCAGG | 4295 | ACAGUUCAUUUCAGG |
| | | | | GTCTCTCAGAAGCTG | | GUCUCUCAGAAGCUG |
| intron1 | | | | | | |
| STMN2_ | + | GTTG | 2290 | TTAACAGTTCATTTCA | 4296 | UUAACAGUUCAUUUCA |
| intron1 | | | | GGGTCTCTCAGAAG | | GGGUCUCUCAGAAG |
| STMN2_ | + | GTTG | 2291 | TTGTTAACAGTTCATT | 4297 | UUGUUAACAGUUCAUU |
| intron1 | | | | TCAGGGTCTCTCAG | | UCAGGGUCUCUCAG |
| STMN2_ | + | ATTC | 2292 | AGTTGTTGTTAACAGT | 4298 | AGUUGUUGUUAACAGU |
| intron1 | | | | TCATTTCAGGGTCT | | UCAUUUCAGGGUCU |
| STMN2_ | + | ATTT | 2293 | ATGAATAGCAATACT | 4299 | AUGAAUAGCAAUACUG |
| intron1 | | | | GAAGAAATTAAAACA | | AAGAAAUUAAAACA |
| STMN2_ | + | GTTA | 2294 | GCCATTCAGTTGTTGT | 4300 | GCCAUUCAGUUGUUGU |
| intron1 | | | | TAACAGTTCATTTC | | UAACAGUUCAUUUC |
| STMN2_ | + | GTTA | 2295 | CTCAACACAAAGTTG | 4301 | CUCAACACAAAGUUGG |
| intron1 | | | | GACTAAGTCTCAAAG | | ACUAAGUCUCAAAG |
| STMN2_ | + | TTTG | 2296 | CAGAATATACTGTTAC | 4302 | CAGAAUAUACUGUUAC |
| intron1 | | | | TCAACACAAAGTTG | | UCAACACAAAGUUG |
| STMN2_ | + | GTTT | 2297 | GCAGAATATACTGTTA | 4303 | GCAGAAUAUACUGUUA |
| intron1 | | | | CTCAACACAAAGTT | | CUCAACACAAAGUU |
| STMN2_ | + | CTTC | 2298 | AGGGTTTGCAGAATA | 4304 | AGGGUUUGCAGAAUAU |
| intron1 | | | | TACTGTTACTCAACA | | ACUGUUACUCAACA |
| STMN2_ | + | TTTC | 2299 | CCAAATAGGGCACTA | 4305 | CCAAAUAGGGCACUAA |
| intron1 | | | | AAAACATGATCCCAA | | AAACAUGAUCCCAA |
| STMN2_ | + | ATTT | 2300 | CCCAAATAGGGCAC | 4306 | CCCAAAUAGGGCACUA |
| intron1 | | | | TAAAAACATGATCCCA | | AAAACAUGAUCCCA |
| STMN2_ | + | ATTA | 2301 | AAAAATATAACATTTC | 4307 | AAAAAUAUAACAUUUC |
| intron1 | | | | CCAAATAGGGCACT | | CCAAAUAGGGCACU |
| STMN2_ | + | ATTA | 2302 | TGCTGCAAAAATGAT | 4308 | UGCUGCAAAAAUGAUA |
| intron1 | | | | ACAATACACGAAATA | | CAAUACACGAAAUA |
| STMN2_ | + | TTTC | 2303 | TGGAAATATTATGCT | 4309 | UGGAAAUAUUAUGCU |
| intron1 | | | | GCAAAAATGATACAA | | GCAAAAAUGAUACAA |
| STMN2_ | + | CTTT | 2304 | CTGGAAATATTATGC | 4310 | CUGGAAAUAUUAUGC |
| intron1 | | | | TGCAAAAATGATACA | | UGCAAAAAUGAUACA |
| STMN2_ | + | ATTA | 2305 | CCACCTTTCTGGAAA | 4311 | CCACCUUUCUGGAAA |
| intron1 | | | | TATTATGCTGCAAAA | | UAUUAUGCUGCAAAA |
| STMN2_ | + | CTTC | 2306 | AAGGAATAGCATCAA | 4312 | AAGGAAUAGCAUCAA |
| | | | | AGACATAGTCAGGTC | | AGACAUAGUCAGGUC |
| intron1 | | | | | | |
| STMN2_ | + | TTTC | 2307 | TAAGGAAGTCAACCT | 4313 | UAAGGAAGUCAACCU |
| intron1 | | | | ACAGATCAGAAAGAG | | ACAGAUCAGAAAGAG |
| STMN2_ | + | GTTG | 2308 | GACTAAGTCTCAAAG | 4314 | GACUAAGUCUCAAAG |
| | | | | TTAGCCATTCAGTTG | | UUAGCCAUUCAGUUG |
| intron1 | | | | | | |
| STMN2_ | + | ATTT | 2309 | CTTGTACATTGAAGG | 4315 | CUUGUACAUUGAAGG |
| intron1 | | | | AAAGGAAGACACACT | | AAAGGAAGACACACU |
| STMN2_ | + | CTTA | 2310 | CTATCATTTATGAAT | 4316 | CUAUCAUUUAUGAAU |
| intron1 | | | | AGCAATACTGAAGAA | | AGCAAUACUGAAGAA |
| STMN2_ | + | CTTG | 2311 | TGGCACAGTTGACAA | 4317 | UGGCACAGUUGACAAG |
| intron1 | | | | GGATGATAAATCAAT | | GAUGAUAAAUCAAU |
| STMN2_ | + | TTTT | 2312 | AGGGATATTAACTTG | 4318 | AGGGAUAUUAACUUGU |
| intron1 | | | | TAATATACAGGTATC | | AAUAUACAGGUAUC |
| STMN2_ | + | GTTT | 2313 | TAGGGATATTAACTTG | 4319 | UAGGGAUAUUAACUUG |
| intron1 | | | | TAATATACAGGTAT | | UAAUAUACAGGUAU |
| STMN2_ | + | ATTC | 2314 | TGACCACTAAACACAT | 4320 | UGACCACUAAACACAU |
| intron1 | | | | CAGTTTTAGGGATA | | CAGUUUUAGGGAUA |
| STMN2_ | + | CTTC | 2315 | CGAACAAGCTCCCAG | 4321 | CGAACAAGCUCCCAGA |
| intron1 | | | | ATGATGCTGATTCTG | | UGAUGCUGAUUCUG |
| STMN2_ | + | ATTC | 2316 | TGTCTTCCGAACAAGC | 4322 | UGUCUUCCGAACAAGC |
| intron1 | | | | TCCCAGATGATGCT | | UCCCAGAUGAUGCU |
| STMN2_ | + | CTTG | 2317 | AGGCAGACATTCTGTC | 4323 | AGGCAGACAUUCUGUC |
| intron1 | | | | TTCCGAACAAGCTC | | UUCCGAACAAGCUC |
| STMN2_ | + | ATTC | 2318 | AATACATCTGGCTTGA | 4324 | AAUACAUCUGGCUUGA |
| intron1 | | | | GGCAGACATTCTGT | | GGCAGACAUUCUGU |
| STMN2_ | + | ATTC | 2319 | TGATTCAATACATCTG | 4325 | UGAUUCAAUACAUCUG |
| intron1 | | | | GCTTGAGGCAGACA | | GCUUGAGGCAGACA |
| STMN2_ | + | ATTA | 2320 | AAATGCAAATTCTGA | 4326 | AAAUGCAAAUUCUGAU |
| intron1 | | | | TTCAATACATCTGGC | | UCAAUACAUCUGGC |
| STMN2_ | + | TTTC | 2321 | ATTAAAATGCAAATT | 4327 | AUUAAAAUGCAAAUU |
| intron1 | | | | CTGATTCAATACATC | | CUGAUUCAAUACAUC |
| STMN2_ | + | TTTT | 2322 | CATTAAAATGCAAAT | 4328 | CAUUAAAAUGCAAAU |
| intron1 | | | | TCTGATTCAATACAT | | UCUGAUUCAAUACAU |
| STMN2_ | + | ATTT | 2323 | TCATTAAAATGCAAA | 4329 | UCAUUAAAAUGCAAAU |
| intron1 | | | | TTCTGATTCAATACA | | UCUGAUUCAAUACA |
| STMN2_ | + | TTTG | 2324 | ATTTTCATTAAAATG | 4330 | AUUUUCAUUAAAAUG |
| intron1 | | | | CAAATTCTGATTCAA | | CAAAUUCUGAUUCAA |
| STMN2_ | + | CTTT | 2325 | GATTTTCATTAAAAT | 4331 | GAUUUUCAUUAAAAUG |
| intron1 | | | | GCAAATTCTGATTCA | | CAAAUUCUGAUUCA |
| STMN2_ | + | ATTA | 2326 | CCTTTGATTTTCATT | 4332 | CCUUUGAUUUUCAUUA |
| intron1 | | | | AAAATGCAAATTCTG | | AAAUGCAAAUUCUG |
| STMN2_ | + | TTTG | 2327 | ATGTGCATATGAATT | 4333 | AUGUGCAUAUGAAUUA |
| intron1 | | | | ACCTTTGATTTTCAT | | CCUUUGAUUUUCAU |
| STMN2_ | + | CTTT | 2328 | GATGTGCATATGAAT | 4334 | GAUGUGCAUAUGAAUU |
| intron1 | | | | TACCTTTGATTTTCA | | ACCUUUGAUUUUCA |
| STMN2_ | + | GTTC | 2329 | CTCAAACTTTGATGT | 4335 | CUCAAACUUUGAUGUG |
| intron1 | | | | GCATATGAATTACCT | | CAUAUGAAUUACCU |
| STMN2_ | + | ATTA | 2330 | CTGTGTTCCTCAAAC | 4336 | CUGUGUUCCUCAAACU |
| intron1 | | | | TTTGATGTGCATATG | | UUGAUGUGCAUAUG |
| STMN2_ | + | TTTA | 2331 | ATAGTGTCATATTAC | 4337 | AUAGUGUCAUAUUAC |
| intron1 | | | | TGTGTTCCTCAAACT | | UGUGUUCCUCAAACU |
| STMN2_ | + | ATTT | 2332 | AATAGTGTCATATTA | 4338 | AAUAGUGUCAUAUUA |
| intron1 | | | | CTGTGTTCCTCAAAC | | CUGUGUUCCUCAAAC |
| STMN2_ | + | ATTC | 2333 | TAATCCAGCTATAAA | 4339 | UAAUCCAGCUAUAAAA |
| intron1 | | | | ATATTTAATAGTGTC | | UAUUUAAUAGUGUC |
| STMN2_ | + | TTTA | 2334 | TGTAATTCTAATCCAG | 4340 | UGUAAUUCUAAUCCAG |
| intron1 | | | | CTATAAAATATTTA | | CUAUAAAAUAUUUA |
| STMN2_ | + | TTTT | 2335 | ATGTAATTCTAATCCA | 4341 | AUGUAAUUCUAAUCCA |
| intron1 | | | | GCTATAAAATATTT | | GCUAUAAAAUAUUU |
| STMN2_ | + | TTTA | 2336 | ATTATCTTTTAATAAA | 4342 | AUUAUCUUUUAAUAAA |
| intron1 | | | | GGAATCAGGCCCTG | | GGAAUCAGGCCCUG |
| STMN2_ | + | ATTT | 2337 | TATGTAATTCTAATCC | 4343 | UAUGUAAUUCUAAUCC |
| intron1 | | | | AGCTATAAAATATT | | AGCUAUAAAAUAUU |
| STMN2_ | + | ATTA | 2338 | CATTTTATGTAATTCT | 4344 | CAUUUUAUGUAAUUCU |
| intron1 | | | | AATCCAGCTATAAA | | AAUCCAGCUAUAAA |
| STMN2_ | + | TTTA | 2339 | GGGATATTAACTTGTA | 4345 | GGGAUAUUAACUUGUA |
| intron1 | | | | ATATACAGGTATCC | | AUAUACAGGUAUCC |
| STMN2_ | + | ATTA | 2340 | ACTTGTAATATACAGG | 4346 | ACUUGUAAUAUACAGG |
| intron1 | | | | TATCCCTCCTGGTA | | UAUCCCUCCUGGUA |
| STMN2_ | + | CTTG | 2341 | TAATATACAGGTATCC | 4347 | UAAUAUACAGGUAUCC |
| intron1 | | | | CTCCTGGTAAGCTC | | CUCCUGGUAAGCUC |
| STMN2_ | + | ATTA | 2342 | TGTCTTAACATTTTTA | 4348 | UGUCUUAACAUUUUUA |
| intron1 | | | | AATCTATGGTAATC | | AAUCUAUGGUAAUC |
| STMN2_ | + | TTTG | 2343 | GCTCTCTGTGTGAGCA | 4349 | GCUCUCUGUGUGAGCA |
| intron1 | | | | TGTGTGCGTGTGTG | | UGUGUGCGUGUGUG |
| STMN2_ | + | ATTT | 2344 | GGCTCTCTGTGTGAGC | 4350 | GGCUCUCUGUGUGAGC |
| intron1 | | | | ATGTGTGCGTGTGT | | AUGUGUGCGUGUGU |
| STMN2_ | + | ATTG | 2345 | CAGGACTCGGCAGAA | 4351 | CAGGACUCGGCAGAAG |
| intron1 | | | | GACCTTCGAGAGAAA | | ACCUUCGAGAGAAA |
| STMN2_ | + | ATTC | 2346 | ATATTGCAGGACTCG | 4352 | AUAUUGCAGGACUCGG |
| intron1 | | | | GCAGAAGACCTTCGA | | CAGAAGACCUUCGA |
| STMN2_ | + | ATTA | 2347 | TATTCATATTGCAGG | 4353 | UAUUCAUAUUGCAGGA |
| intron1 | | | | ACTCGGCAGAAGACC | | CUCGGCAGAAGACC |
| STMN2_ | + | TTTA | 2348 | AAATTATATTCATAT | 4354 | AAAUUAUAUUCAUAUU |
| intron1 | | | | TGCAGGACTCGGCAG | | GCAGGACUCGGCAG |
| STMN2_ | + | TTTT | 2349 | AAAATTATATTCATA | 4355 | AAAAUUAUAUUCAUA |
| intron1 | | | | TTGCAGGACTCGGCA | | UUGCAGGACUCGGCA |
| STMN2_ | + | TTTT | 2350 | TAAAATTATATTCAT | 4356 | UAAAAUUAUAUUCAU |
| intron1 | | | | ATTGCAGGACTCGGC | | AUUGCAGGACUCGGC |
| STMN2_ | + | ATTT | 2351 | TTAAAATTATATTCA | 4357 | UUAAAAUUAUAUUCA |
| intron1 | | | | TATTGCAGGACTCGG | | UAUUGCAGGACUCGG |
| STMN2_ | + | ATTG | 2352 | GATTTTTAAAATTAT | 4358 | GAUUUUUAAAAUUAU |
| intron1 | | | | ATTCATATTGCAGGA | | AUUCAUAUUGCAGGA |
| STMN2_ | + | CTTA | 2353 | ATTGGATTTTTAAAA | 4359 | AUUGGAUUUUUAAAA |
| intron1 | | | | TTATATTCATATTGC | | UUAUAUUCAUAUUGC |
| STMN2_ | + | GTTC | 2354 | TGCCCCATCACTCTC | 4360 | UGCCCCAUCACUCUC |
| intron1 | | | | TCTTAATTGGATTTT | | UCUUAAUUGGAUUUU |
| STMN2_ | + | ATTA | 2355 | TGTGTTCTGCCCCAT | 4361 | UGUGUUCUGCCCCAUC |
| intron1 | | | | CACTCTCTCTTAATT | | ACUCUCUCUUAAUU |
| STMN2_ | + | GTTG | 2356 | ACAAGGATGATAAAT | 4362 | ACAAGGAUGAUAAAUC |
| intron1 | | | | CAATAATGCAAGCTT | | AAUAAUGCAAGCUU |
| STMN2_ | + | ATTA | 2357 | CTCTGGGAATTATGTG | 4363 | CUCUGGGAAUUAUGUG |
| intron1 | | | | TTCTGCCCCATCAC | | UUCUGCCCCAUCAC |
| STMN2_ | + | TTTT | 2358 | ATTACTCTGGGAATT | 4364 | AUUACUCUGGGAAUUA |
| intron1 | | | | ATGTGTTCTGCCCCA | | UGUGUUCUGCCCCA |
| STMN2_ | + | ATTT | 2359 | TATTACTCTGGGAAT | 4365 | UAUUACUCUGGGAAUU |
| intron1 | | | | TATGTGTTCTGCCCC | | AUGUGUUCUGCCCC |
| STMN2_ | + | CTTC | 2360 | CGAACTCATATACCTG | 4366 | CGAACUCAUAUACCUG |
| intron1 | | | | GGGATTTTATTACT | | GGGAUUUUAUUACU |
| STMN2_ | + | TTTA | 2361 | CTTCCGAACTCATAT | 4367 | CUUCCGAACUCAUAUA |
| intron1 | | | | ACCTGGGGATTTTAT | | CCUGGGGAUUUUAU |
| STMN2_ | + | TTTT | 2362 | ACTTCCGAACTCATA | 4368 | ACUUCCGAACUCAUAU |
| intron1 | | | | TACCTGGGGATTTTA | | ACCUGGGGAUUUUA |
| STMN2_ | + | ATTT | 2363 | TACTTCCGAACTCAT | 4369 | UACUUCCGAACUCAUA |
| intron1 | | | | ATACCTGGGGATTTT | | UACCUGGGGAUUUU |
| STMN2_ | + | TTTA | 2364 | CAAAATATTTTACTT | 4370 | CAAAAUAUUUUACUUC |
| intron1 | | | | CCGAACTCATATACC | | CGAACUCAUAUACC |
| STMN2_ | + | CTTT | 2365 | ACAAAATATTTTACT | 4371 | ACAAAAUAUUUUACUU |
| intron1 | | | | TCCGAACTCATATAC | | CCGAACUCAUAUAC |
| STMN2_ | + | TTTA | 2366 | AATCTATGGTAATCT | 4372 | AAUCUAUGGUAAUCUU |
| intron1 | | | | TTACAAAATATTTTA | | UACAAAAUAUUUUA |
| STMN2_ | + | TTTT | 2367 | AAATCTATGGTAATC | 4373 | AAAUCUAUGGUAAUC |
| intron1 | | | | TTTACAAAATATTTT | | UUUACAAAAUAUUUU |
| STMN2_ | + | TTTT | 2368 | TAAATCTATGGTAAT | 4374 | UAAAUCUAUGGUAAUC |
| intron1 | | | | CTTTACAAAATATTT | | UUUACAAAAUAUUU |
| STMN2_ | + | ATTT | 2369 | TTAAATCTATGGTAA | 4375 | UUAAAUCUAUGGUAA |
| intron1 | | | | TCTTTACAAAATATT | | UCUUUACAAAAUAUU |
| STMN2_ | + | CTTA | 2370 | ACATTTTTAAATCTA | 4376 | ACAUUUUUAAAUCUAU |
| intron1 | | | | TGGTAATCTTTACAA | | GGUAAUCUUUACAA |
| STMN2_ | + | TTTA | 2371 | TTACTCTGGGAATTA | 4377 | UUACUCUGGGAAUUAU |
| intron1 | | | | TGTGTTCTGCCCCAT | | GUGUUCUGCCCCAU |
| STMN2_ | + | TTTC | 2372 | TTGTACATTGAAGGA | 4378 | UUGUACAUUGAAGGAA |
| intron1 | | | | AAGGAAGACACACTT | | AGGAAGACACACUU |
| STMN2_ | + | CTTC | 2373 | GAGAGAAAGGTAGAA | 4379 | GAGAGAAAGGUAGAA |
| intron1 | | | | AATAAGAATTTGGCT | | AAUAAGAAUUUGGCU |
| STMN2_ | + | ATTG | 2374 | AAGGAAAGGAAGACA | 4380 | AAGGAAAGGAAGACA |
| intron1 | | | | CACTTAAGACAGCAT | | CACUUAAGACAGCAU |
| STMN2_ | + | CTTA | 2375 | ATCTCCTCAGTCCCA | 4381 | AUCUCCUCAGUCCCA |
| intron1 | | | | TCATGGTTAGCACAT | | UCAUGGUUAGCACAU |
| STMN2_ | + | ATTG | 2376 | ACTTAATCTCCTCAG | 4382 | ACUUAAUCUCCUCAG |
| intron1 | | | | TCCCATCATGGTTAG | | UCCCAUCAUGGUUAG |
| STMN2_ | + | GTTC | 2377 | CAGAAATAACATTGA | 4383 | CAGAAAUAACAUUGAC |
| intron1 | | | | CTTAATCTCCTCAGT | | UUAAUCUCCUCAGU |
| STMN2_ | + | TTTC | 2378 | TGGTGGGAACACACT | 4384 | UGGUGGGAACACACU |
| intron1 | | | | CTGATGACCAGTTCC | | CUGAUGACCAGUUCC |
| STMN2_ | + | ATTT | 2379 | CTGGTGGGAACACAC | 4385 | CUGGUGGGAACACACU |
| intron1 | | | | TCTGATGACCAGTTC | | CUGAUGACCAGUUC |
| STMN2_ | + | GTTC | 2380 | TGCAGGCTCAGCACA | 4386 | UGCAGGCUCAGCACAG |
| intron1 | | | | GCATCGATTTCTGGT | | CAUCGAUUUCUGGU |
| STMN2_ | + | GTTG | 2381 | TAACGTATGAGACAC | 4387 | UAACGUAUGAGACACA |
| intron1 | | | | ATGGCGTTCTGCAGG | | UGGCGUUCUGCAGG |
| STMN2_ | + | TTTG | 2382 | GGAGAAAGAGAGCTA | 4388 | GGAGAAAGAGAGCUAU |
| intron1 | | | | TGAGGCCGTGTGGGT | | GAGGCCGUGUGGGU |
| STMN2_ | + | CTTT | 2383 | GGGAGAAAGAGAGCT | 4389 | GGGAGAAAGAGAGCUA |
| intron1 | | | | ATGAGGCCGTGTGGG | | UGAGGCCGUGUGGG |
| STMN2_ | + | TTTA | 2384 | GGCTTTGGGAGAAAG | 4390 | GGCUUUGGGAGAAAGA |
| intron1 | | | | AGAGCTATGAGGCCG | | GAGCUAUGAGGCCG |
| STMN2_ | + | ATTT | 2385 | AGGCTTTGGGAGAAA | 4391 | AGGCUUUGGGAGAAAG |
| intron1 | | | | GAGAGCTATGAGGCC | | AGAGCUAUGAGGCC |
| STMN2_ | + | ATTG | 2386 | CCATGATTTAGGCTTT | 4392 | CCAUGAUUUAGGCUUU |
| intron1 | | | | GGGAGAAAGAGAGC | | GGGAGAAAGAGAGC |
| STMN2_ | + | ATTC | 2387 | AAATAATTGCCATGAT | 4393 | AAAUAAUUGCCAUGAU |
| intron1 | | | | TTAGGCTTTGGGAG | | UUAGGCUUUGGGAG |
| STMN2_ | + | CTTA | 2388 | TTCAAATAATTGCCAT | 4394 | UUCAAAUAAUUGCCAU |
| intron1 | | | | GATTTAGGCTTTGG | | GAUUUAGGCUUUGG |
| STMN2_ | + | CTTA | 2389 | CCTGGGGCTTATTCAA | 4395 | CCUGGGGCUUAUUCAA |
| intron1 | | | | ATAATTGCCATGAT | | AUAAUUGCCAUGAU |
| STMN2_ | | TTTA | 2390 | ATAGCTTACCTGGGGC | 4396 | AUAGCUUACCUGGGGC |
| intron1 | | | | TTATTCAAATAATT | | UUAUUCAAAUAAUU |
| STMN2_ | + | TTTT | 2391 | AATAGCTTACCTGGG | 4397 | AAUAGCUUACCUGGG |
| intron1 | | | | GCTTATTCAAATAAT | | GCUUAUUCAAAUAAU |
| STMN2_ | + | GTTT | 2392 | TAATAGCTTACCTGG | 4398 | UAAUAGCUUACCUGG |
| intron1 | | | | GGCTTATTCAAATAA | | GGCUUAUUCAAAUAA |
| STMN2_ | + | ATTG | 2393 | ATGCCTAGTTTTAAT | 4399 | AUGCCUAGUUUUAAUA |
| intron1 | | | | AGCTTACCTGGGGCT | | GCUUACCUGGGGCU |
| STMN2_ | + | CTTC | 2394 | CAAATTGATGCCTAGT | 4400 | CAAAUUGAUGCCUAGU |
| intron1 | | | | TTTAATAGCTTACC | | UUUAAUAGCUUACC |
| STMN2_ | + | CTTG | 2395 | AAGAGAAAATACTTC | 4401 | AAGAGAAAAUACUUCC |
| intron1 | | | | CAAATTGATGCCTAG | | AAAUUGAUGCCUAG |
| STMN2_ | + | TTTC | 2396 | TGATCACAGACTCAC | 4402 | UGAUCACAGACUCACC |
| intron1 | | | | CTTGAAGAGAAAATA | | UUGAAGAGAAAAUA |
| STMN2_ | + | CTTT | 2397 | CTGATCACAGACTCA | 4403 | CUGAUCACAGACUCAC |
| intron1 | | | | CCTTGAAGAGAAAAT | | CUUGAAGAGAAAAU |
| STMN2_ | + | CTTC | 2398 | TCCTTTCTGATCACA | 4404 | UCCUUUCUGAUCACAG |
| intron1 | | | | GACTCACCTTGAAGA | | ACUCACCUUGAAGA |
| STMN2_ | + | TTTA | 2399 | AACAGACCAGAGATG | 4405 | AACAGACCAGAGAUGG |
| intron1 | | | | GTCTTCTCCTTTCTG | | UCUUCUCCUUUCUG |
| STMN2_ | + | ATTT | 2400 | AAACAGACCAGAGAT | 4406 | AAACAGACCAGAGAUG |
| intron1 | | | | GGTCTTCTCCTTTCT | | GUCUUCUCCUUUCU |
| STMN2_ | + | TTTA | 2401 | TTTAAACAGACCAGA | 4407 | UUUAAACAGACCAGAG |
| intron1 | | | | GATGGTCTTCTCCTT | | AUGGUCUUCUCCUU |
| STMN2_ | + | GTTA | 2402 | GCACATTTCAAAATG | 4408 | GCACAUUUCAAAAUGC |
| intron1 | | | | CCTCCTTAACTACTT | | CUCCUUAACUACUU |
| STMN2_ | + | TTTT | 2403 | TAATTATCTTTTAAT | 4409 | UAAUUAUCUUUUAAUA |
| intron1 | | | | AAAGGAATCAGGCCC | | AAGGAAUCAGGCCC |
| STMN2_ | + | TTTC | 2404 | AAAATGCCTCCTTAA | 4410 | AAAAUGCCUCCUUAAC |
| intron1 | | | | CTACTTCCATAGGCC | | UACUUCCAUAGGCC |
| STMN2_ | + | CTTA | 2405 | ACTACTTCCATAGGC | 4411 | ACUACUUCCAUAGGCC |
| intron1 | | | | CAGAGATATTTAGTT | | AGAGAUAUUUAGUU |
| STMN2_ | + | ATTT | 2406 | TTAATTATCTTTTAAT | 4412 | UUAAUUAUCUUUUAAU |
| intron1 | | | | AAAGGAATCAGGCC | | AAAGGAAUCAGGCC |
| STMN2_ | + | CTTG | 2407 | TGAAACATTTTTAAT | 4413 | UGAAACAUUUUUAAUU |
| intron1 | | | | TATCTTTTAATAAAG | | AUCUUUUAAUAAAG |
| STMN2_ | + | CTTG | 2408 | TACATTGAAGGAAAG | 4414 | UACAUUGAAGGAAAGG |
| intron1 | | | | GAAGACACACTTAAG | | AAGACACACUUAAG |
| STMN2_ | + | TTTG | 2409 | AATCCCTTGTGAAAC | 4415 | AAUCCCUUGUGAAAC |
| intron1 | | | | ATTTTTAATTATCTT | | AUUUUUAAUUAUCUU |
| STMN2_ | + | TTTT | 2410 | GAATCCCTTGTGAAA | 4416 | GAAUCCCUUGUGAAAC |
| intron1 | | | | CATTTTTAATTATCT | | AUUUUUAAUUAUCU |
| STMN2_ | + | GTTT | 2411 | TGAATCCCTTGTGAA | 4417 | UGAAUCCCUUGUGAAA |
| intron1 | | | | ACATTTTTAATTATC | | CAUUUUUAAUUAUC |
| STMN2_ | + | ATTA | 2412 | CCATCAAAGCAGGCA | 4418 | CCAUCAAAGCAGGCAG |
| intron1 | | | | GGCAGGCAGGAGAGA | | GCAGGCAGGAGAGA |
| STMN2_ | + | CTTC | 2413 | ATATTACCATCAAAG | 4419 | AUAUUACCAUCAAAGC |
| intron1 | | | | CAGGCAGGCAGGCAG | | AGGCAGGCAGGCAG |
| STMN2_ | + | ATTC | 2414 | TCTTCATATTACCAT | 4420 | UCUUCAUAUUACCAUC |
| intron1 | | | | CAAAGCAGGCAGGCA | | AAAGCAGGCAGGCA |
| STMN2_ | + | TTTC | 2415 | AAGATTCTCTTCATA | 4421 | AAGAUUCUCUUCAUA |
| intron1 | | | | TTACCATCAAAGCAG | | UUACCAUCAAAGCAG |
| STMN2_ | + | ATTT | 2416 | CAAGATTCTCTTCAT | 4422 | CAAGAUUCUCUUCAUA |
| intron1 | | | | ATTACCATCAAAGCA | | UUACCAUCAAAGCA |
| STMN2_ | + | GTTA | 2417 | TTTCAAGATTCTCTT | 4423 | UUUCAAGAUUCUCUUC |
| intron1 | | | | CATATTACCATCAAA | | AUAUUACCAUCAAA |
| STMN2_ | + | ATTA | 2418 | GATGTTATTTCAAGA | 4424 | GAUGUUAUUUCAAGAU |
| intron1 | | | | TTCTCTTCATATTAC | | UCUCUUCAUAUUAC |
| STMN2_ | + | TTTT | 2419 | ATTTAAACAGACCAG | 4425 | AUUUAAACAGACCAG |
| intron1 | | | | AGATGGTCTTCTCCT | | AGAUGGUCUUCUCCU |
| STMN2_ | + | TTTA | 2420 | ATATAACTATTAGAT | 4426 | AUAUAACUAUUAGAU |
| intron1 | | | | GTTATTTCAAGATTC | | GUUAUUUCAAGAUUC |
| STMN2_ | + | ATTC | 2421 | ACATTTAATATAACT | 4427 | ACAUUUAAUAUAACU |
| intron1 | | | | ATTAGATGTTATTTC | | AUUAGAUGUUAUUUC |
| STMN2_ | + | TTTA | 2422 | CACATTCACATTTAA | 4428 | CACAUUCACAUUUAA |
| intron1 | | | | TATAACTATTAGATG | | UAUAACUAUUAGAUG |
| STMN2_ | + | ATTT | 2423 | ACACATTCACATTTA | 4429 | ACACAUUCACAUUUA |
| intron1 | | | | ATATAACTATTAGAT | | AUAUAACUAUUAGAU |
| STMN2_ | + | GTTG | 2424 | AATAAAATAAATTTA | 4430 | AAUAAAAUAAAUUUA |
| intron1 | | | | CACATTCACATTTAA | | CACAUUCACAUUUAA |
| STMN2_ | + | TTTG | 2425 | TTGAATAAAATAAAT | 4431 | UUGAAUAAAAUAAAU |
| intron1 | | | | TTACACATTCACATT | | UUACACAUUCACAUU |
| STMN2_ | + | TTTT | 2426 | GTTGAATAAAATAAA | 4432 | GUUGAAUAAAAUAAA |
| intron1 | | | | TTTACACATTCACAT | | UUUACACAUUCACAU |
| STMN2_ | + | ATTT | 2427 | TGTTGAATAAAATAA | 4433 | UGUUGAAUAAAAUAA |
| intron1 | | | | ATTTACACATTCACA | | AUUUACACAUUCACA |
| STMN2_ | + | TTTA | 2428 | ACATTTTGTTGAATA | 4434 | ACAUUUUGUUGAAUA |
| intron1 | | | | AAATAAATTTACACA | | AAAUAAAUUUACACA |
| STMN2_ | + | TTTT | 2429 | AACATTTTGTTGAAT | 4435 | AACAUUUUGUUGAAU |
| intron1 | | | | AAAATAAATTTACAC | | AAAAUAAAUUUACAC |
| STMN2_ | + | GTTT | 2430 | TAACATTTTGTTGAA | 4436 | UAACAUUUUGUUGAA |
| intron1 | | | | TAAAATAAATTTACA | | UAAAAUAAAUUUACA |
| STMN2_ | + | TTTA | 2431 | GTTTTAACATTTTGT | 4437 | GUUUUAACAUUUUGU |
| intron1 | | | | TGAATAAAATAAATT | | UGAAUAAAAUAAAUU |
| STMN2_ | + | ATTT | 2432 | AGTTTTAACATTTTG | 4438 | AGUUUUAACAUUUUG |
| intron1 | | | | TTGAATAAAATAAAT | | UUGAAUAAAAUAAAU |
| STMN2_ | + | CTTC | 2433 | CATAGGCCAGAGATA | 4439 | CAUAGGCCAGAGAUA |
| intron1 | | | | TTTAGTTTTAACATT | | UUUAGUUUUAACAUU |
| STMN2_ | + | ATTT | 2434 | AATATAACTATTAGA | 4440 | AAUAUAACUAUUAGA |
| intron1 | | | | TGTTATTTCAAGATT | | UGUUAUUUCAAGAUU |
| STMN2_ | + | ATTT | 2435 | TATTTAAACAGACCA | 4441 | UAUUUAAACAGACCA |
| intron1 | | | | GAGATGGTCTTCTCC | | GAGAUGGUCUUCUCC |
| STMN2_ | + | ATTT | 2436 | CAAAATGCCTCCTTA | 4442 | CAAAAUGCCUCCUUA |
| intron1 | | | | ACTACTTCCATAGGC | | ACUACUUCCAUAGGC |
| STMN2_ | + | GTTA | 2437 | GAGGTGAGCTCCCAT | 4443 | GAGGUGAGCUCCCAU |
| intron1 | | | | TGCAGAGGTCACACC | | UGCAGAGGUCACACC |
| STMN2_ | + | TTTC | 2438 | TGGTGTATTCATAAA | 4444 | UGGUGUAUUCAUAAA |
| intron1 | | | | TTCCAGATTCTCTAT | | UUCCAGAUUCUCUAU |
| STMN2_ | + | TTTT | 2439 | CTGGTGTATTCATAA | 4445 | CUGGUGUAUUCAUAA |
| intron1 | | | | ATTCCAGATTCTCTA | | AUUCCAGAUUCUCUA |
| STMN2_ | + | TTTT | 2440 | TCTGGTGTATTCATA | 4446 | UCUGGUGUAUUCAUA |
| intron1 | | | | AATTCCAGATTCTCT | | AAUUCCAGAUUCUCU |
| STMN2_ | + | GTTT | 2441 | TTCTGGTGTATTCAT | 4447 | UUCUGGUGUAUUCAU |
| intron1 | | | | AAATTCCAGATTCTC | | AAAUUCCAGAUUCUC |
| STMN2_ | + | TTTC | 2442 | AACTGTTTTTCTGGT | 4448 | AACUGUUUUUCUGGU |
| intron1 | | | | GTATTCATAAATTCC | | GUAUUCAUAAAUUCC |
| STMN2_ | 1 | CTTT | 2443 | CAACTGTTTTTCTGG | 4449 | CAACUGUUUUUCUGGU |
| intron1 | | | | TGTATTCATAAATTC | | GUAUUCAUAAAUUC |
| STMN2_ | 1 | TTTC | 2444 | TTTCAACTGTTTTTC | 4450 | UUUCAACUGUUUUUCU |
| intron1 | | | | TGGTGTATTCATAAA | | GGUGUAUUCAUAAA |
| STMN2_ | + | CTTT | 2445 | CTTTCAACTGTTTTT | 4451 | CUUUCAACUGUUUUUC |
| intron1 | | | | CTGGTGTATTCATAA | | UGGUGUAUUCAUAA |
| STMN2_ | + | TTTC | 2446 | CCGCAATGGTGCTTT | 4452 | CCGCAAUGGUGCUUUC |
| intron1 | | | | CTTTCAACTGTTTTT | | UUUCAACUGUUUUU |
| STMN2_ | + | TTTT | 2447 | CCCGCAATGGTGCTT | 4453 | CCCGCAAUGGUGCUUU |
| intron1 | | | | TCTTTCAACTGTTTT | | CUUUCAACUGUUUU |
| STMN2_ | + | ATTT | 2448 | TCCCGCAATGGTGCT | 4454 | UCCCGCAAUGGUGCUU |
| intron1 | | | | TTCTTTCAACTGTTT | | UCUUUCAACUGUUU |
| STMN2_ | + | TTTA | 2449 | CTCAAACATTTTCCCG | 4455 | CUCAAACAUUUUCCCG |
| intron1 | | | | CAATGGTGCTTTCT | | CAAUGGUGCUUUCU |
| STMN2_ | + | TTTC | 2450 | TTTACTCAAACATTTT | 4456 | UUUACUCAAACAUUUU |
| intron1 | | | | CCCGCAATGGTGCT | | CCCGCAAUGGUGCU |
| STMN2_ | + | ATTC | 2451 | ATAAATTCCAGATTCT | 4457 | AUAAAUUCCAGAUUCU |
| intron1 | | | | CTATGGGAAGTAAC | | CUAUGGGAAGUAAC |
| STMN2_ | + | ATTT | 2452 | CTTTACTCAAACATTT | 4458 | CUUUACUCAAACAUUU |
| intron1 | | | | TCCCGCAATGGTGC | | UCCCGCAAUGGUGC |
| STMN2_ | + | CTTG | 2453 | AGGGCCTCGAGCCAA | 4459 | AGGGCCUCGAGCCAAU |
| intron1 | | | | TAAGTCTTCCTATTT | | AAGUCUUCCUAUUU |
| STMN2_ | + | TTTG | 2454 | GAGATGACAAAAATC | 4460 | GAGAUGACAAAAAUCU |
| intron1 | | | | TAAACTTGAGGGCCT | | AAACUUGAGGGCCU |
| STMN2_ | + | ATTT | 2455 | GGAGATGACAAAAAT | 4461 | GGAGAUGACAAAAAUC |
| intron1 | | | | CTAAACTTGAGGGCC | | UAAACUUGAGGGCC |
| STMN2_ | + | ATTC | 2456 | TGGCAGTCGGGCAGG | 4462 | UGGCAGUCGGGCAGGG |
| intron1 | | | | GCTCTCTGTATAACC | | CUCUCUGUAUAACC |
| STMN2_ | + | TTTA | 2457 | ATTCTGGCAGTCGGG | 4463 | AUUCUGGCAGUCGGGC |
| intron1 | | | | CAGGGCTCTCTGTAT | | AGGGCUCUCUGUAU |
| STMN2_ | + | GTTT | 2458 | AATTCTGGCAGTCGG | 4464 | AAUUCUGGCAGUCGGG |
| intron1 | | | | GCAGGGCTCTCTGTA | | CAGGGCUCUCUGUA |
| STMN2_ | + | TTTA | 2459 | AATGTTTAATTCTGG | 4465 | AAUGUUUAAUUCUGGC |
| intron1 | | | | CAGTCGGGCAGGGCT | | AGUCGGGCAGGGCU |
| STMN2_ | + | TTTT | 2460 | AAATGTTTAATTCTG | 4466 | AAAUGUUUAAUUCUGG |
| intron1 | | | | GCAGTCGGGCAGGGC | | CAGUCGGGCAGGGC |
| STMN2_ | + | GTTT | 2461 | TAAATGTTTAATTCT | 4467 | UAAAUGUUUAAUUCUG |
| intron1 | | | | GGCAGTCGGGCAGGG | | GCAGUCGGGCAGGG |
| STMN2_ | + | ATT | 2462 | CAGAGGTCACACCTGT | 4468 | CAGAGGUCACACCUGU |
| intron1 | | G | | GATATCACCATTTT | | GAUAUCACCAUUUU |
| STMN2_ | + | ATTC | 2463 | ATGTTTTAAATGTTT | 4469 | AUGUUUUAAAUGUUU |
| intron1 | | | | AATTCTGGCAGTCGG | | AAUUCUGGCAGUCGG |
| STMN2_ | + | ATTA | 2464 | CAAAAGTAATTCATG | 4470 | CAAAAGUAAUUCAUG |
| intron1 | | | | TTTTAAATGTTTAAT | | UUUUAAAUGUUUAAU |
| STMN2_ | + | CTTA | 2465 | AGACAGCATTACAAA | 4471 | AGACAGCAUUACAAAA |
| intron1 | | | | AGTAATTCATGTTTT | | GUAAUUCAUGUUUU |
| STMN2_ | + | CTTC | 2466 | CTATTTCTTTACTCAA | 4472 | CUAUUUCUUUACUCAA |
| intron1 | | | | ACATTTTCCCGCAA | | ACAUUUUCCCGCAA |
| STMN2_ | + | ATTC | 2467 | CAGATTCTCTATGGGA | 4473 | CAGAUUCUCUAUGGGA |
| intron1 | | | | AGTAACTTTTATTG | | AGUAACUUUUAUUG |
| STMN2_ | + | CTTT | 2468 | ACTCAAACATTTTCC | 4474 | ACUCAAACAUUUUCCC |
| intron1 | | | | CGCAATGGTGCTTTC | | GCAAUGGUGCUUUC |
| STMN2_ | + | CTTA | 2469 | CTCAAGGTCACACAGT | 4475 | CUCAAGGUCACACAGU |
| intron1 | | | | TAGTCAGATCCAGA | | UAGUCAGAUCCAGA |
| STMN2_ | + | ATTC | 2470 | TCTATGGGAAGTAACT | 4476 | UCUAUGGGAAGUAACU |
| intron1 | | | | TTTATTGATTGATT | | UUUAUUGAUUGAUU |
| STMN2_ | + | TTTC | 2471 | ACCGATTGCTGCTAG | 4477 | ACCGAUUGCUGCUAGU |
| intron1 | | | | TCTCATATCTGTTCC | | CUCAUAUCUGUUCC |
| STMN2_ | + | CTTT | 2472 | CACCGATTGCTGCTA | 4478 | CACCGAUUGCUGCUAG |
| intron1 | | | | GTCTCATATCTGTTC | | UCUCAUAUCUGUUC |
| STMN2_ | + | CTTC | 2473 | GGAATCCATCTTTCAC | 4479 | GGAAUCCAUCUUUCAC |
| intron1 | | | | CGATTGCTGCTAGT | | CGAUUGCUGCUAGU |
| STMN2_ | + | TTTG | 2474 | GGCCCAGGCCATCTG | 4480 | GGCCCAGGCCAUCUGG |
| intron1 | | | | GCTTCGGAATCCATC | | CUUCGGAAUCCAUC |
| STMN2_ | + | ATTT | 2475 | GGGCCCAGGCCATCT | 4481 | GGGCCCAGGCCAUCUG |
| intron1 | | | | GGCTTCGGAATCCAT | | GCUUCGGAAUCCAU |
| STMN2_ | + | GTTA | 2476 | GTCAGATCCAGAATTT | 4482 | GUCAGAUCCAGAAUUU |
| intron1 | | | | GGGCCCAGGCCATC | | GGGCCCAGGCCAUC |
| STMN2_ | + | GTTG | 2477 | AAGTATCTTACTCAAG | 4483 | AAGUAUCUUACUCAAG |
| intron1 | | | | GTCACACAGTTAGT | | GUCACACAGUUAGU |
| STMN2_ | + | ATTA | 2478 | CAGATATGGAAACTG | 4484 | CAGAUAUGGAAACUGA |
| intron1 | | | | AGGCACAGAAAGTTG | | GGCACAGAAAGUUG |
| STMN2_ | + | GTTC | 2479 | TATTACAGATATGGA | 4485 | UAUUACAGAUAUGGAA |
| intron1 | | | | AACTGAGGCACAGAA | | ACUGAGGCACAGAA |
| STMN2_ | + | ATTG | 2480 | CTGCTAGTCTCATATC | 4486 | CUGCUAGUCUCAUAUC |
| intron1 | | | | TGTTCCATGTTAGA | | UGUUCCAUGUUAGA |
| STMN2_ | + | GTTA | 2481 | ATCACTTAATAATCCT | 4487 | AUCACUUAAUAAUCCU |
| intron1 | | | | AAGTAGGTTCTATT | | AAGUAGGUUCUAUU |
| STMN2_ | + | GTTC | 2482 | CATGTTAGAGGTGAG | 4488 | CAUGUUAGAGGUGAGC |
| intron1 | | | | CTCCCATTGCAGAGG | | UCCCAUUGCAGAGG |
| STMN2_ | + | CTTA | 2483 | ATAATCCTAAGTAGG | 4489 | AUAAUCCUAAGUAGGU |
| intron1 | | | | TTCTATTACAGATAT | | UCUAUUACAGAUAU |
| STMN2_ | + | TTTC | 2484 | CACATATTAACTGTG | 4490 | CACAUAUUAACUGUGU |
| intron1 | | | | TTAATCACTTAATAA | | UAAUCACUUAAUAA |
| STMN2_ | + | CTTT | 2485 | TATTGATTGATTTAA | 4491 | UAUUGAUUGAUUUAA |
| intron1 | | | | CCCTTGTATAGCACA | | CCCUUGUAUAGCACA |
| STMN2_ | + | TTTT | 2486 | ATTGATTGATTTAAC | 4492 | AUUGAUUGAUUUAAC |
| intron1 | | | | CCTTGTATAGCACAT | | CCUUGUAUAGCACAU |
| STMN2_ | + | TTTA | 2487 | TTGATTGATTTAACC | 4493 | UUGAUUGAUUUAACCC |
| intron1 | | | | CTTGTATAGCACATA | | UUGUAUAGCACAUA |
| STMN2_ | + | ATTG | 2488 | ATTGATTTAACCCTTG | 4494 | AUUGAUUUAACCCUUG |
| intron1 | | | | TATAGCACATATAA | | UAUAGCACAUAUAA |
| STMN2_ | + | ATTG | 2489 | ATTTAACCCTTGTATA | 4495 | AUUUAACCCUUGUAUA |
| intron1 | | | | GCACATATAACATG | | GCACAUAUAACAUG |
| STMN2_ | + | ATTA | 2490 | ACTGTGTTAATCACTT | 4496 | ACUGUGUUAAUCACUU |
| intron1 | | | | AATAATCCTAAGTA | | AAUAAUCCUAAGUA |
| STMN2_ | + | ATTT | 2491 | AACCCTTGTATAGCAC | 4497 | AACCCUUGUAUAGCAC |
| intron1 | | | | ATATAACATGCAAG | | AUAUAACAUGCAAG |
| STMN2_ | + | CTTG | 2492 | TATAGCACATATAAC | 4498 | UAUAGCACAUAUAACA |
| intron1 | | | | ATGCAAGGCATTGTT | | UGCAAGGCAUUGUU |
| STMN2_ | + | ATTG | 2493 | TTCTAAGAACTTTCCA | 4499 | UUCUAAGAACUUUCCA |
| intron1 | | | | CATATTAACTGTGT | | CAUAUUAACUGUGU |
| STMN2_ | + | GTTC | 2494 | TAAGAACTTTCCACA | 4500 | UAAGAACUUUCCACA |
| intron1 | | | | TATTAACTGTGTTAA | | UAUUAACUGUGUUAA |
| STMN2_ | + | CTTT | 2495 | CCACATATTAACTGT | 4501 | CCACAUAUUAACUGU |
| intron1 | | | | GTTAATCACTTAATA | | GUUAAUCACUUAAUA |
| STMN2_ | + | TTTA | 2496 | ACCCTTGTATAGCAC | 4502 | ACCCUUGUAUAGCAC |
| intron1 | | | | ATATAACATGCAAGG | | AUAUAACAUGCAAGG |

* The three 3′ nucleotides represent the 5′−TTN−3′ motif.

The present disclosure includes all combinations of the direct repeat sequences and spacer sequences listed above, consistent with the present disclosure herein.

In some embodiments, a spacer sequence described herein comprises a uracil (U). In some embodiments, a spacer sequence described herein comprises a thymine (T). In some embodiments, a spacer sequence according to Table 5A or 5B comprises a sequence comprising a thymine in one or more (e.g., all) places indicated as uracil in Table 5A or 5B.

The present disclosure includes RNA guides that comprise any and all combinations of the direct repeats and spacers described herein (e.g., as set forth in Table 5A or 5B, above).

In some embodiments, the RNA guide has at least 90% identity (e.g., at least 90%, 91%, 92%, 93%, 94%, 95%, 96%, 97%, 98% or 99% identity to any one of SEQ ID NOs: 4505-4562. In some embodiments, the RNA guide has at least 95% identity (e.g., at least 95%, 96%, 97%, 98% or 99% identity to any one of SEQ ID NOs: 4505-4562. In some embodiments, the RNA guide has a sequence set forth in any one of SEQ ID NOs: 4505-4562.

In some embodiments, the RNA guide comprises the sequence of any one of SEQ ID NOs: 4508, 4512, 4559, and 4561, or the second nucleic acid encodes an RNA guide comprising any one of SEQ ID NOs: 4508, 4512, 4559, and 4561.

In some embodiments, the RNA guide comprises the sequence of any one of SEQ ID NOs: 4505, 4506, 4507, 4508, 4509, 4510, 4511, 4512, 4513, 4514, 4515, 4554, 4555, 4556, 4557, 4558, 4559, 4560, 4561, and 4562, or the second nucleic acid encodes an RNA guide comprising any one of SEQ ID NOs: 4505, 4506, 4507, 4508, 4509, 4510, 4511, 4512, 4513, 4514, 4515, 4554, 4555, 4556, 4557, 4558, 4559, 4560, 4561, and 4562.

### B. Nucleic Acid Modifications

The RNA guide may include one or more covalent modifications with respect to a reference sequence, in particular the parent polyribonucleotide, which are included within the scope of this disclosure.

Exemplary modifications can include any modification to the sugar, the nucleobase, the internucleoside linkage (e.g., to a linking phosphate/to a phosphodiester linkage/to the phosphodiester backbone), and any combination thereof. Some of the exemplary modifications provided herein are described in detail below.

The RNA guide may include any useful modification, such as to the sugar, the nucleobase, or the internucleoside linkage (e.g., to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210.

Different sugar modifications, nucleotide modifications, and/or internucleoside linkages (e.g., backbone structures) may exist at various positions in the sequence. One of ordinary skill in the art will appreciate that the nucleotide analogs or other modification(s) may be located at any position(s) of the sequence, such that the function of the sequence is not substantially decreased. The sequence may include from about 1% to about 100% modified nucleotides (either in relation to overall nucleotide content, or in relation to one or more types of nucleotide, i.e. any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, sugar modifications (e.g., at the 2′ position or 4′ position) or replacement of the sugar at one or more ribonucleotides of the sequence may, as well as backbone modifications, include modification or replacement of the phosphodiester linkages. Specific examples of a sequence include, but are not limited to, sequences including modified backbones or no natural internucleoside linkages such as internucleoside modifications, including modification or replacement of the phosphodiester linkages. Sequences having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, a sequence will include ribonucleotides with a phosphorus atom in its internucleoside backbone.

Modified sequence backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the sequence may be negatively or positively charged.

The modified nucleotides, which may be incorporated into the sequence, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment.

In specific embodiments, a modified nucleoside includes an alpha-thio-nucleoside (e.g., 5′-O-(1-thiophosphate)-adenosine, 5′-O-(1-thiophosphate)-cytidine (a-thio-cytidine), 5′-O-(1-thiophosphate)-guanosine, 5′-O-(1-thiophosphate)-uridine, or 5′-O-(1-thiophosphate)-pseudouridine).

Other internucleoside linkages that may be employed according to the present disclosure, including internucleoside linkages which do not contain a phosphorous atom, are described herein.

In some embodiments, the sequence may include one or more cytotoxic nucleosides. For example, cytotoxic nucleosides may be incorporated into sequence, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-2,4 (1H,3H)-dione), troxacitabine, tezacitabine, 2′-deoxy-2′-methylidenecytidine (DMDC), and 6-mercaptopurine. Additional examples include fludarabine phosphate, N4-behenyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester).

In some embodiments, the sequence includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc.). The one or more post-transcriptional modifications can be any post-transcriptional modification, such as any of the more than one hundred different nucleoside modifications that have been identified in RNA (Rozenski, J, Crain, P, and McCloskey, J. (1999). The RNA Modification Database: 1999 update. Nucl Acids Res 27:196-197) In some embodiments, the first isolated nucleic acid comprises messenger RNA (mRNA). In some embodiments, the mRNA comprises at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

The sequence may or may not be uniformly modified along the entire length of the molecule. For example, one or more or all types of nucleotides (e.g., naturally-occurring nucleotides, purine or pyrimidine, or any one or more or all of A, G, U, C, I, pU) may or may not be uniformly modified in the sequence, or in a given predetermined sequence region thereof. In some embodiments, the sequence includes a pseudouridine. In some embodiments, the sequence includes an inosine, which may aid in the immune system characterizing the sequence as endogenous versus viral RNAs. The incorporation of inosine may also mediate improved RNA stability/reduced degradation. See for example, Yu, Z. et al. (2015) RNA editing by ADAR1 marks dsRNA as “self”. Cell Res. 25, 1283-1284, which is incorporated by reference in its entirety.

In some embodiments, one or more of the nucleotides of an RNA guide comprises a 2′-O-methyl phosphorothioate modification. In some embodiments, each of the first three nucleotides of the RNA guide comprises a 2′-O-methyl phosphorothioate modification. In some embodiments, each of the last four nucleotides of the RNA guide comprises a 2′-O-methyl phosphorothioate modification. In some embodiments, each of the first to last, second to last, and third to last nucleotides of the RNA guide comprises a 2′-O-methyl phosphorothioate modification, and wherein the last nucleotide of the RNA guide is unmodified. In some embodiments, each of the first three nucleotides of the RNA guide comprises a 2′-O-methyl phosphorothioate modification, and each of the first to last, second to last, and third to last nucleotides of the RNA guide comprises a 2′-O-methyl phosphorothioate modification.

When a gene editing system disclosed herein comprises nucleic acids encoding the Cas12i polypeptide disclosed herein, e.g., mRNA molecules, such nucleic acid molecules may contain any of the modifications disclosed herein, where applicable.

### C. Cas12i Polypeptide

In some embodiments, the composition or system of the present disclosure includes a Cas12i polypeptide as described in WO/2019/178427, the relevant disclosures of which are incorporated by reference for the subject matter and purpose referenced herein.

In some embodiments, the genetic editing system of the present disclosure comprises a Cas12i2 polypeptide described herein (e.g., a polypeptide comprising SEQ ID NO: 448 and/or encoded by SEQ ID NO: 447 (or a version thereof in which T's are replaced with U's)). In some embodiments, the Cas12i2 polypeptide comprises at least one RuvC domain. In some embodiments, the genetic editing system of the present disclosure comprises a nucleic acid molecule (e.g., a DNA molecule or a polyribonucleotide molecule) encoding a Cas12i polypeptide.

A nucleic acid sequence encoding the Cas12i2 polypeptide described herein may be substantially identical to a reference nucleic acid sequence, e.g., SEQ ID NO: 447 (or a version thereof in which T's are replaced with U's). In some embodiments, the Cas12i2 polypeptide is encoded by a nucleic acid comprising a sequence having least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence, e.g., SEQ ID NO: 447 (or a version thereof in which T's are replaced with U's). The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the nucleic acid molecules hybridize to the complementary sequence of the other under stringent conditions of temperature and ionic strength (e.g., within a range of medium to high stringency). See, e.g., Tijssen, “Hybridization with Nucleic Acid Probes. Part I. Theory and Nucleic Acid Preparation” (Laboratory Techniques in Biochemistry and Molecular Biology, Vol 24).

In some embodiments, the Cas12i2 polypeptide is encoded by a nucleic acid sequence having at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more sequence identity, but not 100% sequence identity, to a reference nucleic acid sequence, e.g., SEQ ID NO: 447 (or a version thereof in which T's are replaced with U's).

In some embodiments, the Cas12i2 polypeptide of the present disclosure comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 448.

In some embodiments, the present disclosure describes a Cas12i2 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 448. Homology or identity can be determined by amino acid sequence alignment, e.g., using a program such as BLAST, ALIGN, or CLUSTAL, as described herein.

Also provided is a Cas12i2 polypeptide of the present disclosure having enzymatic activity, e.g., nuclease or endonuclease activity, and comprising an amino acid sequence which differs from the amino acid sequences of SEQ ID NO: 448 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the Cas12i2 polypeptide may contain one or more mutations relative to SEQ ID NO: 448, for example, at position D581, G624, F626, P868, I926, V1030, E1035, S1046, or any combination thereof. In some instances, the one or more mutations are amino acid substitutions, for example, D581R, G624R, F626R, P868T, 1926R, V1030G, E1035R, S1046G, or a combination thereof.

In some embodiments, the Cas12i2 polypeptide comprises a polypeptide having a sequence of SEQ ID NO: 449, SEQ ID NO: 450, SEQ ID NO: 451, SEQ ID NO: 452, or SEQ ID NO: 453. In some examples, the Cas12i2 polypeptide contains mutations at positions D581, D911, I926, and V1030. Such a Cas12i2 polypeptide may contain amino acid substitutions of D581R, D911R, I926R, and V1030G (e.g., SEQ ID NO: 449). In some examples, the Cas12i2 polypeptide contains mutations at positions D581, I926, and V1030. Such a Cas12i2 polypeptide may contain amino acid substitutions of D581R, 1926R, and V1030G (e.g., SEQ ID NO: 450). In some examples, the Cas12i2 polypeptide may contain mutations at positions D581, I926, V1030, and S1046. Such a Cas12i2 polypeptide may contain amino acid substitutions of D581R, 1926R, V1030G, and S1046G (e.g., SEQ ID NO: 451). In some examples, the Cas12i2 polypeptide may contain mutations at positions D581, G624, F626, I926, V1030, E1035, and S1046. Such a Cas12i2 polypeptide may contain amino acid substitutions of D581R, G624R. F626R, I926R, V1030G, E1035R, and S1046G (e.g., SEQ ID NO: 452). In some examples, the Cas12i2 polypeptide may contain mutations at positions D581, G624, F626, P868, I926, V1030, E1035, and S1046. Such a Cas12i2 polypeptide may contain amino acid substitutions of D581R. G624R, F626R. P868T, 1926R, V1030G, E1035R, and S1046G (e.g., SEQ ID NO: 453).

In some embodiments, the Cas12i2 polypeptide of the present disclosure comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 449, SEQ ID NO: 450, SEQ ID NO: 451, SEQ ID NO: 452, or SEQ ID NO: 453. In some embodiments, a Cas12i2 polypeptide having at least 50%, 60%, 65%, 70%, 75%, 80%, 85%, 90%, 91%, 92%, 93%, 94%, 95%, 96%, 97%, 98%, 99%, or 100% identity to SEQ ID NO: 449, SEQ ID NO: 450, SEQ ID NO: 451, SEQ ID NO: 452, or SEQ ID NO: 453 maintains the amino acid changes (or at least 1, 2, 3 etc. of these changes) that differentiate the polypeptide from its respective parent/reference sequence.

In some embodiments, the present disclosure describes a Cas12i2 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 449, SEQ ID NO: 450, SEQ ID NO: 451, SEQ ID NO: 452, or SEQ ID NO: 453. Homology or identity can be determined by amino acid sequence alignment, e.g., using a program such as BLAST, ALIGN, or CLUSTAL, as described herein.

Also provided is a Cas12i2 polypeptide of the present disclosure having enzymatic activity, e.g., nuclease or endonuclease activity, and comprising an amino acid sequence which differs from the amino acid sequences of SEQ ID NO: 449, SEQ ID NO: 450, SEQ ID NO: 451, SEQ ID NO: 452, or SEQ ID NO: 453 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the composition of the present disclosure includes a Cas12i4 polypeptide described herein (e.g., a polypeptide comprising SEQ ID NO: 482 and/or encoded by SEQ ID NO: 481 (or a version thereof in which T's are replaced with U's)). In some embodiments, the Cas12i4 polypeptide comprises at least one RuvC domain.

A nucleic acid sequence encoding the Cas12i4 polypeptide described herein may be substantially identical to a reference nucleic acid sequence, e.g., SEQ ID NO: 481 (or a version thereof in which T's are replaced with U's). In some embodiments, the Cas12i4 polypeptide is encoded by a nucleic acid comprising a sequence having least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence, e.g., SEQ ID NO: 481 (or a version thereof in which T's are replaced with U's). The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the nucleic acid molecules hybridize to the complementary sequence of the other under stringent conditions of temperature and ionic strength (e.g., within a range of medium to high stringency).

In some embodiments, the Cas12i4 polypeptide is encoded by a nucleic acid sequence having at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more sequence identity, but not 100% sequence identity, to a reference nucleic acid sequence, e.g., SEQ ID NO: 481 (or a version thereof in which T's are replaced with U's).

In some embodiments, the Cas12i4 polypeptide of the present disclosure comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 482.

In some embodiments, the present disclosure describes a Cas12i4 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 482. Homology or identity can be determined by amino acid sequence alignment, e.g., using a program such as BLAST, ALIGN, or CLUSTAL, as described herein.

Also provided is a Cas12i4 polypeptide of the present disclosure having enzymatic activity, e.g., nuclease or endonuclease activity, and comprising an amino acid sequence which differs from the amino acid sequences of SEQ ID NO: 482 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the Cas12i4 polypeptide comprises a polypeptide having a sequence of SEQ ID NO: 483 or SEQ ID NO: 484.

In some embodiments, the Cas12i4 polypeptide of the present disclosure comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 483 or SEQ ID NO: 484. In some embodiments, a Cas12i4 polypeptide having at least 50%, 60%, 65%, 70%, 75%, 80%, 85%, 90%, 91%, 92%, 93%, 94%, 95%, 96%, 97%, 98%, 99%, or 100% identity to SEQ ID NO: 483 or SEQ ID NO: 484 maintains the amino acid changes (or at least 1, 2, 3 etc. of these changes) that differentiate it from its respective parent/reference sequence.

In some embodiments, the present disclosure describes a Cas12i4 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 483 or SEQ ID NO: 484. Homology or identity can be determined by amino acid sequence alignment, e.g., using a program such as BLAST, ALIGN, or CLUSTAL, as described herein.

Also provided is a Cas12i4 polypeptide of the present disclosure having enzymatic activity, e.g., nuclease or endonuclease activity, and comprising an amino acid sequence which differs from the amino acid sequences of SEQ ID NO: 483 or SEQ ID NO: 484 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the composition of the present disclosure includes a Cas12i1 polypeptide described herein (e.g., a polypeptide comprising SEQ ID NO: 4503). In some embodiments, the Cas12i1 polypeptide comprises at least one RuvC domain.

In some embodiments, the Cas12i1 polypeptide of the present disclosure comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 4503.

In some embodiments, the present disclosure describes a Cas12i1 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 4503. Homology or identity can be determined by amino acid sequence alignment, e.g., using a program such as BLAST, ALIGN, or CLUSTAL, as described herein.

Also provided is a Cas12i1 polypeptide of the present disclosure having enzymatic activity, e.g., nuclease or endonuclease activity, and comprising an amino acid sequence which differs from the amino acid sequences of SEQ ID NO: 4503 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the composition of the present disclosure includes a Cas12i3 polypeptide described herein (e.g., a polypeptide comprising SEQ ID NO: 4504). In some embodiments, the Cas1213 polypeptide comprises at least one RuvC domain.

In some embodiments, the Cas12i3 polypeptide of the present disclosure comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 4504.

In some embodiments, the present disclosure describes a Cas1213 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 4504. Homology or identity can be determined by amino acid sequence alignment, e.g., using a program such as BLAST, ALIGN, or CLUSTAL, as described herein.

Also provided is a Cas12i3 polypeptide of the present disclosure having enzymatic activity, e.g., nuclease or endonuclease activity, and comprising an amino acid sequence which differs from the amino acid sequences of SEQ ID NO: 4504 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

Although the changes described herein may be one or more amino acid changes, changes to the Cas12i polypeptide may also be of a substantive nature, such as fusion of polypeptides as amino- and/or carboxyl-terminal extensions. For example, the Cas12i polypeptide may contain additional peptides, e.g., one or more peptides. Examples of additional peptides may include epitope peptides for labelling, such as a polyhistidine tag (His-tag), Myc, and FLAG. In some embodiments, the Cas12i polypeptide described herein can be fused to a detectable moiety such as a fluorescent protein (e.g., green fluorescent protein (GFP) or yellow fluorescent protein (YFP)).

In some embodiments, the Cas12i polypeptide comprises at least one (e.g., two, three, four, five, six, or more) nuclear localization signal (NLS). In some embodiments, the Cas12i polypeptide comprises at least one (e.g., two, three, four, five, six, or more) nuclear export signal (NES). In some embodiments, the Cas12i polypeptide comprises at least one (e.g., two, three, four, five, six, or more) NLS and at least one (e.g., two, three, four, five, six, or more) NES.

In some embodiments, the Cas12i polypeptide described herein can be self-inactivating. See, Epstein et al., “Engineering a Self-Inactivating CRISPR System for AAV Vectors,” Mol. Ther., 24 (2016): S50, which is incorporated by reference in its entirety.

In some embodiments, the nucleotide sequence encoding the Cas12i polypeptide described herein can be codon-optimized for use in a particular host cell or organism. For example, the nucleic acid can be codon-optimized for any non-human eukaryote including mice, rats, rabbits, dogs, livestock, or non-human primates. Codon usage tables are readily available, for example, at the “Codon Usage Database” available at the world wide web site of kazusa.orjp/codon/and these tables can be adapted in a number of ways. Sec Nakamura et al. Nucl. Acids Res. 28:292 (2000), which is incorporated herein by reference in its entirety. Computer algorithms for codon optimizing a particular sequence for expression in a particular host cell are also available, such as Gene Forge (Aptagen; Jacobus, PA). In some examples, the nucleic acid encoding the Cas12i polypeptides such as Cas12i2 polypeptides as disclosed herein can be an mRNA molecule, which can be codon optimized.

Exemplary Cas12i polypeptide sequences and corresponding nucleotide sequences are listed in Table 7.

TABLE 7
Cas12i and STMN2 Sequences

| SEQ ID | | |
|---|---|---|
| NO: | Sequence | Description |
| 447 | ATGAGCAGCGCGATCAAAAGCTACAAGAGCGTTCTGCGTCCGAAC | Nucleotide |
| | GAGCGTAAGAACCAACTGCTGAAAAGCACCATTCAGTGCCTGGAA | sequence |
| | GACGGTAGCGCGTTCTTTTTCAAGATGCTGCAAGGCCTGTTTGGT | encoding |
| | GGCATCACCCCGGAGATTGTTCGTTTCAGCACCGAACAGGAGAAA | Cas12i2 |
| | CAGCAACAGGATATCGCGCTGTGGTGCGCGGTTAACTGGTTCCGT | |
| | CCGGTGAGCCAAGACAGCCTGACCCACACCATTGCGAGCGATAAC | |
| | CTGGTGGAGAAGTTTGAGGAATACTATGGTGGCACCGCGAGCGAC | |
| | GCGATCAAACAGTACTTCAGCGCGAGCATTGGCGAAAGCTACTAT | |
| | TGGAACGACTGCCGTCAACAGTACTATGATCTGTGCCGTGAGCTG | |
| | GGTGTTGAGGTGAGCGACCTGACCCATGATCTGGAGATCCTGTGC | |
| | CGTGAAAAGTGCCTGGCGGTTGCGACCGAGAGCAACCAGAACAAC | |
| | AGCATCATTAGCGTTCTGTTTGGCACCGGCGAAAAAGAGGACCGT | |
| | AGCGTGAAACTGCGTATCACCAAGAAAATTCTGGAGGCGATCAGC | |
| | AACCTGAAAGAAATCCCGAAGAACGTTGCGCCGATTCAAGAGATC | |
| | ATTCTGAACGTGGCGAAAGCGACCAAGGAAACCTTCCGTCAGGTG | |
| | TATGCGGGTAACCTGGGTGCGCCGAGCACCCTGGAGAAATTTATC | |
| | GCGAAGGACGGCCAAAAAGAGTTCGATCTGAAGAAACTGCAGACC | |
| | GACCTGAAGAAAGTTATTCGTGGTAAAAGCAAGGAGCGTGATTGG | |
| | TGCTGCCAGGAAGAGCTGCGTAGCTACGTGGAGCAAAACACCATC | |
| | CAGTATGACCTGTGGGCGTGGGGCGAAATGTTCAACAAAGCGCAC | |
| | ACCGCGCTGAAAATCAAGAGCACCCGTAACTACAACTTTGCGAAG | |
| | CAACGTCTGGAACAGTTCAAAGAGATTCAGAGCCTGAACAACCTG | |
| | CTGGTTGTGAAGAAGCTGAACGACTTTTTCGATAGCGAATTTTTC | |
| | AGCGGCGAGGAAACCTACACCATCTGCGTTCACCATCTGGGTGGC | |
| | AAGGACCTGAGCAAACTGTATAAGGCGTGGGAGGATGATCCGGCG | |
| | GACCCGGAAAACGCGATTGTGGTTCTGTGCGACGATCTGAAAAAC | |
| | AACTTTAAGAAAGAGCCGATCCGTAACATTCTGCGTTACATCTTC | |
| | ACCATTCGTCAAGAATGCAGCGCGCAGGACATCCTGGCGGCGGCG | |
| | AAGTACAACCAACAGCTGGATCGTTATAAAAGCCAAAAGGCGAAC | |
| | CCGAGCGTTCTGGGTAACCAGGGCTTTACCTGGACCAACGCGGTG | |
| | ATCCTGCCGGAGAAGGCGCAGCGTAACGACCGTCCGAACAGCCTG | |
| | GATCTGCGTATTTGGCTGTACCTGAAACTGCGTCACCCGGACGGT | |
| | CGTTGGAAGAAACACCATATCCCGTTCTACGATACCCGTTTCTTC | |
| | CAAGAAATTTATGCGGCGGGCAACAGCCCGGTTGACACCTGCCAG | |
| | TTTCGTACCCCGCGTTTCGGTTATCACCTGCCGAAACTGACCGAT | |
| | CAGACCGCGATCCGTGTTAACAAGAAACATGTGAAAGCGGCGAAG | |
| | ACCGAGGCGCGTATTCGTCTGGCGATCCAACAGGGCACCCTGCCG | |
| | GTGAGCAACCTGAAGATCACCGAAATTAGCGCGACCATCAACAGC | |
| | AAAGGTCAAGTGCGTATTCCGGTTAAGTTTGACGTGGGTCGTCAA | |
| | AAAGGCACCCTGCAGATCGGTGACCGTTTCTGCGGCTACGATCAA | |
| | AACCAGACCGCGAGCCACGCGTATAGCCTGTGGGAAGTGGTTAAA | |
| | GAGGGTCAATACCATAAAGAGCTGGGCTGCTTTGTTCGTTTCATC | |
| | AGCAGCGGTGACATCGTGAGCATTACCGAGAACCGTGGCAACCAA | |
| | TTTGATCAGCTGAGCTATGAAGGTCTGGCGTACCCGCAATATGCG | |
| | GACTGGCGTAAGAAAGCGAGCAAGTTCGTGAGCCTGTGGCAGATC | |
| | ACCAAGAAAAACAAGAAAAAGGAAATCGTGACCGTTGAAGCGAAA | |
| | GAGAAGTTTGACGCGATCTGCAAGTACCAGCCGCGTCTGTATAAA | |
| | TTCAACAAGGAGTACGCGTATCTGCTGCGTGATATTGTTCGTGGC | |
| | AAAAGCCTGGTGGAACTGCAACAGATTCGTCAAGAGATCTTTCGT | |
| | TTCATTGAACAGGACTGCGGTGTTACCCGTCTGGGCAGCCTGAGC | |
| | CTGAGCACCCTGGAAACCGTGAAAGCGGTTAAGGGTATCATTTAC | |
| | AGCTATTTTAGCACCGCGCTGAACGCGAGCAAGAACAACCCGATC | |
| | AGCGACGAACAGCGTAAAGAGTTTGATCCGGAACTGTTCGCGCTG | |
| | CTGGAAAAGCTGGAGCTGATTCGTACCCGTAAAAAGAAACAAAAA | |
| | GTGGAACGTATCGCGAACAGCCTGATTCAGACCTGCCTGGAGAAC | |
| | AACATCAAGTTCATTCGTGGTGAAGGCGACCTGAGCACCACCAAC | |
| | AACGCGACCAAGAAAAAGGCGAACAGCCGTAGCATGGATTGGTTG | |
| | GCGCGTGGTGTTTTTAACAAAATCCGTCAACTGGCGCCGATGCAC | |
| | AACATTACCCTGTTCGGTTGCGGCAGCCTGTACACCAGCCACCAG | |
| | GACCCGCTGGTGCATCGTAACCCGGATAAAGCGATGAAGTGCCGT | |
| | TGGGCGGCGATCCCGGTTAAGGACATTGGCGATTGGGTGCTGCGT | |
| | AAGCTGAGCCAAAACCTGCGTGCGAAAAACATCGGCACCGGCGAG | |
| | TACTATCACCAAGGTGTTAAAGAGTTCCTGAGCCATTATGAACTG | |
| | CAGGACCTGGAGGAAGAGCTGCTGAAGTGGCGTAGCGATCGTAAA | |
| | AGCAACATTCCGTGCTGGGTGCTGCAGAACCGTCTGGCGGAGAAG | |
| | CTGGGCAACAAAGAAGCGGTGGTTTACATCCCGGTTCGTGGTGGC | |
| | CGTATTTATTTTGCGACCCACAAGGTGGCGACCGGTGCGGTGAGC | |
| | ATCGTTTTCGACCAAAAACAAGTGTGGGTTTGCAACGCGGATCAT | |
| | GTTGCGGCGGCGAACATCGCGCTGACCGTGAAGGGTATTGGCGAA | |
| | CAAAGCAGCGACGAAGAGAACCCGGATGGTAGCCGTATCAAACTG | |
| | CAGCTGACCAGC | |
| 448 | MSSAIKSYKSVLRPNERKNQLLKSTIQCLEDGSAFFFKMLQGLFG | Cas12i2 |
| | GITPEIVRFSTEQEKQQQDIALWCAVNWFRPVSQDSLTHTIASDN | amino acid |
| | LVEKFEEYYGGTASDAIKQYFSASIGESYYWNDCRQQYYDLCREL | sequence |
| | GVEVSDLTHDLEILCREKCLAVATESNQNNSIISVLFGTGEKEDR | |
| | SVKLRITKKILEAISNLKEIPKNVAPIQEIILNVAKATKETFRQV | |
| | YAGNLGAPSTLEKFIAKDGQKEFDLKKLQTDLKKVIRGKSKERDW | |
| | CCQEELRSYVEQNTIQYDLWAWGEMFNKAHTALKIKSTRNYNFAK | |
| | QRLEQFKEIQSLNNLLVVKKLNDFFDSEFFSGEETYTICVHHLGG | |
| | KDLSKLYKAWEDDPADPENAIVVLCDDLKNNFKKEPIRNILRYIF | |
| | TIRQECSAQDILAAAKYNQQLDRYKSQKANPSVLGNQGFTWTNAV | |
| | ILPEKAQRNDRPNSLDLRIWLYLKLRHPDGRWKKHHIPFYDTRFF | |
| | QEIYAAGNSPVDTCQFRTPRFGYHLPKLTDQTAIRVNKKHVKAAK | |
| | TEARIRLAIQQGTLPVSNLKITEISATINSKGQVRIPVKFDVGRQ | |
| | KGTLQIGDRFCGYDQNQTASHAYSLWEVVKEGQYHKELGCFVRFI | |
| | SSGDIVSITENRGNQFDQLSYEGLAYPQYADWRKKASKFVSLWQI | |
| | TKKNKKKEIVTVEAKEKFDAICKYQPRLYKFNKEYAYLLRDIVRG | |
| | KSLVELQQIRQEIFRFIEQDCGVTRLGSLSLSTLETVKAVKGIIY | |
| | SYFSTALNASKNNPISDEQRKEFDPELFALLEKLELIRTRKKKQK | |
| | VERIANSLIQTCLENNIKFIRGEGDLSTINNATKKKANSRSMDWL | |
| | ARGVFNKIRQLAPMHNITLFGCGSLYTSHQDPLVHRNPDKAMKCR | |
| | WAAIPVKDIGDWVLRKLSQNLRAKNIGTGEYYHQGVKEFLSHYEL | |
| | QDLEEELLKWRSDRKSNIPCWVLQNRLAEKLGNKEAVVYIPVRGG | |
| | RIYFATHKVATGAVSIVFDQKQVWVCNADHVAAANIALTVKGIGE | |
| | QSSDEENPDGSRIKLQLTS | |
| 449 | MSSAIKSYKS VLRPNERKNQ LLKSTIQCLE DGSAFFFKML | Variant |
| | QGLFGGITPE IVRESTEQEK QQQDIALWCA VNWFRPVSQD | Cas12i2 of |
| | SLTHTIASDN LVEKFEEYYG GTASDAIKQY FSASIGESYY | SEQ ID NO: |
| | WNDCRQQYYD LCRELGVEVS DLTHDLEILC REKCLAVATE | 3 of |
| | SNQNNSIISV LFGTGEKEDR SVKLRITKKI LEAISNLKEI | PCT/US2021/ |
| | PKNVAPIQEI ILNVAKATKE TFRQVYAGNL GAPSTLEKFI | 025257 |
| | AKDGQKEFDL KKLQTDLKKV IRGKSKERDW CCQEELRSYV | |
| | EQNTIQYDLW AWGEMFNKAH TALKIKSTRN YNFAKQRLEQ | |
| | FKEIQSLNNL LVVKKLNDFF DSEFFSGEET YTICVHHLGG | |
| | KDLSKLYKAW EDDPADPENA IVVLCDDLKN NFKKEPIRNI | |
| | LRYIFTIRQE CSAQDILAAA KYNQQLDRYK SQKANPSVLG | |
| | NQGFTWTNAV ILPEKAQRND RPNSLDLRIW LYLKLRHPDG | |
| | RWKKHHIPFY DTRFFQEIYA AGNSPVDTCQ FRTPRFGYHL | |
| | PKLTDQTAIR VNKKHVKAAK TEARIRLAIQ QGTLPVSNLK | |
| | ITEISATINS KGQVRIPVKF RVGRQKGTLQ IGDRFCGYDQ | |
| | NQTASHAYSL WEVVKEGQYH KELGCFVRFI SSGDIVSITE | |
| | NRGNQFDQLS YEGLAYPQYA DWRKKASKFV SLWQITKKNK | |
| | KKEIVTVEAK EKFDAICKYQ PRLYKENKEY AYLLRDIVRG | |
| | KSLVELQQIR QEIFRFIEQD CGVTRLGSLS LSTLETVKAV | |
| | KGIIYSYFST ALNASKNNPI SDEQRKEFDP ELFALLEKLE | |
| | LIRTRKKKQK VERIANSLIQ TCLENNIKFI RGEGDLSTTN | |
| | NATKKKANSR SMDWLARGVF NKIRQLAPMH NITLFGCGSL | |
| | YTSHQDPLVH RNPDKAMKCR WAAIPVKDIG RWVLRKLSQN | |
| | LRAKNRGTGE YYHQGVKEFL SHYELQDLEE ELLKWRSDRK | |
| | SNIPCWVLQN RLAEKLGNKE AVVYIPVRGG RIYFATHKVA | |
| | TGAVSIVFDQ KQVWVCNADH VAAANIALTG KGIGEQSSDE | |
| | ENPDGSRIKL QLTS | |
| 450 | MSSAIKSYKS VLRPNERKNQ LLKSTIQCLE DGSAFFFKML | Variant |
| | QGLEGGITPE IVRESTEQEK QQQDIALWCA VNWFRPVSQD | Cas12i2 of |
| | SLTHTIASDN LVEKFEEYYG GTASDAIKQY FSASIGESYY | SEQ ID NO: |
| | WNDCRQQYYD LCRELGVEVS DLTHDLEILC REKCLAVATE | 4 of |
| | SNQNNSIISV LFGTGEKEDR SVKLRITKKI LEAISNLKEI | PCT/US2021/ |
| | PKNVAPIQEI ILNVAKATKE TFRQVYAGNL GAPSTLEKFI | 025257 |
| | AKDGQKEFDL KKLQTDLKKV IRGKSKERDW CCQEELRSYV | |
| | EQNTIQYDLW AWGEMFNKAH TALKIKSTRN YNFAKQRLEQ | |
| | FKEIQSLNNL LVVKKLNDFF DSEFFSGEET YTICVHHLGG | |
| | KDLSKLYKAW EDDPADPENA IVVLCDDLKN NFKKEPIRNI | |
| | LRYIFTIRQE CSAQDILAAA KYNQQLDRYK SQKANPSVLG | |
| | NQGFTWTNAV ILPEKAQRND RPNSLDLRIW LYLKLRHPDG | |
| | RWKKHHIPFY DTRFFQEIYA AGNSPVDTCQ FRTPRFGYHL | |
| | PKLTDQTAIR VNKKHVKAAK TEARIRLAIQ QGTLPVSNLK | |
| | ITEISATINS KGQVRIPVKF RVGRQKGTLQ IGDRFCGYDQ | |
| | NQTASHAYSL WEVVKEGQYH KELGCFVRFI SSGDIVSITE | |
| | NRGNQFDQLS YEGLAYPQYA DWRKKASKFV SLWQITKKNK | |
| | KKEIVTVEAK EKFDAICKYQ PRLYKFNKEY AYLLRDIVRG | |
| | KSLVELQQIR QEIFRFIEQD CGVTRLGSLS LSTLETVKAV | |
| | KGIIYSYFST ALNASKNNPI SDEQRKEFDP ELFALLEKLE | |
| | LIRTRKKKQK VERIANSLIQ TCLENNIKFI RGEGDLSTIN | |
| | NATKKKANSR SMDWLARGVF NKIRQLAPMH NITLFGCGSL | |
| | YTSHQDPLVH RNPDKAMKCR WAAIPVKDIG DWVLRKLSQN | |
| | LRAKNRGTGE YYHQGVKEFL SHYELQDLEE ELLKWRSDRK | |
| | SNIPCWVLQN RLAEKLGNKE AVVYIPVRGG RIYFATHKVA | |
| | TGAVSIVFDQ KQVWVCNADH VAAANIALTG KGIGEQSSDE | |
| | ENPDGSRIKL QLTS | |
| 451 | MSSAIKSYKS VLRPNERKNQ LLKSTIQCLE DGSAFFFKML | Variant |
| | QGLFGGITPE IVRESTEQEK QQQDIALWCA VNWFRPVSQD | Cas12i2 of |
| | SLTHTIASDN LVEKFEEYYG GTASDAIKQY FSASIGESYY | SEQ ID NO: |
| | WNDCRQQYYD LCRELGVEVS DLTHDLEILC REKCLAVATE | 5 of |
| | SNQNNSIISV LFGTGEKEDR SVKLRITKKI LEAISNLKEI | PCT/US2021/ |
| | PKNVAPIQEI ILNVAKATKE TFRQVYAGNL GAPSTLEKFI | 025257 |
| | AKDGQKEFDL KKLQTDLKKV IRGKSKERDW CCQEELRSYV | |
| | EQNTIQYDLW AWGEMFNKAH TALKIKSTRN YNFAKQRLEQ | |
| | FKEIQSLNNL LVVKKLNDFF DSEFFSGEET YTICVHHLGG | |
| | KDLSKLYKAW EDDPADPENA IVVLCDDLKN NFKKEPIRNI | |
| | LRYIFTIRQE CSAQDILAAA KYNQQLDRYK SQKANPSVLG | |
| | NQGFTWTNAV ILPEKAQRND RPNSLDLRIW LYLKLRHPDG | |
| | RWKKHHIPFY DTRFFQEIYA AGNSPVDTCQ FRTPRFGYHL | |
| | PKLTDQTAIR VNKKHVKAAK TEARIRLAIQ QGTLPVSNLK | |
| | ITEISATINS KGQVRIPVKF RVGRQKGTLQ IGDRFCGYDQ | |
| | NQTASHAYSL WEVVKEGQYH KELGCFVRFI SSGDIVSITE | |
| | NRGNQFDQLS YEGLAYPQYA DWRKKASKFV SLWQITKKNK | |
| | KKEIVTVEAK EKFDAICKYQ PRLYKFNKEY AYLLRDIVRG | |
| | KSLVELQQIR QEIFRFIEQD CGVTRLGSLS LSTLETVKAV | |
| | KGIIYSYFST ALNASKNNPI SDEQRKEFDP ELFALLEKLE | |
| | LIRTRKKKQK VERIANSLIQ TCLENNIKFI RGEGDLSTTN | |
| | NATKKKANSR SMDWLARGVF NKIRQLAPMH NITLFGCGSL | |
| | YTSHQDPLVH RNPDKAMKCR WAAIPVKDIG DWVLRKLSQN | |
| | LRAKNRGTGE YYHQGVKEFL SHYELQDLEE ELLKWRSDRK | |
| | SNIPCWVLQN RLAEKLGNKE AVVYIPVRGG RIYFATHKVA | |
| | TGAVSIVFDQ KQVWVCNADH VAAANIALTG KGIGEQSSDE | |
| | ENPDGGRIKL QLTS | |
| 452 | MSSAIKSYKS VLRPNERKNQ LLKSTIQCLE DGSAFFFKML | Variant |
| | QGLFGGITPE IVRESTEQEK QQQDIALWCA VNWFRPVSQD | Cas12i2 of |
| | SLTHTIASDN LVEKFEEYYG GTASDAIKQY FSASIGESYY | SEQ ID NO: |
| | WNDCRQQYYD LCRELGVEVS DLTHDLEILC REKCLAVATE | 495 of |
| | SNQNNSIISV LFGTGEKEDR SVKLRITKKI LEAISNLKEI | PCT/US2021/ |
| | PKNVAPIQEI ILNVAKATKE TFRQVYAGNL GAPSTLEKFI | 025257 |
| | AKDGQKEFDL KKLQTDLKKV IRGKSKERDW CCQEELRSYV | |
| | EQNTIQYDLW AWGEMENKAH TALKIKSTRN YNFAKQRLEQ | |
| | FKEIQSLNNL LVVKKLNDFF DSEFFSGEET YTICVHHLGG | |
| | KDLSKLYKAW EDDPADPENA IVVLCDDLKN NFKKEPIRNI | |
| | LRYIFTIRQE CSAQDILAAA KYNQQLDRYK SQKANPSVLG | |
| | NQGFTWTNAV ILPEKAQRND RPNSLDLRIW LYLKLRHPDG | |
| | RWKKHHIPFY DTRFFQEIYA AGNSPVDTCQ FRTPRFGYHL | |
| | PKLTDQTAIR VNKKHVKAAK TEARIRLAIQ QGTLPVSNLK | |
| | ITEISATINS KGQVRIPVKF RVGRQKGTLQ IGDRFCGYDQ | |
| | NQTASHAYSL WEVVKEGQYH KELRCRVRFI SSGDIVSITE | |
| | NRGNQFDQLS YEGLAYPQYA DWRKKASKFV SLWQITKKNK | |
| | KKEIVTVEAK EKFDAICKYQ PRLYKFNKEY AYLLRDIVRG | |
| | KSLVELQQIR QEIFRFIEQD CGVTRLGSLS LSTLETVKAV | |
| | KGIIYSYFST ALNASKNNPI SDEQRKEFDP ELFALLEKLE | |
| | LIRTRKKKQK VERIANSLIQ TCLENNIKFI RGEGDLSTIN | |
| | NATKKKANSR SMDWLARGVF NKIRQLAPMH NITLFGCGSL | |
| | YTSHQDPLVH RNPDKAMKCR WAAIPVKDIG DWVLRKLSQN | |
| | LRAKNRGTGE YYHQGVKEFL SHYELQDLEE ELLKWRSDRK | |
| | SNIPCWVLQN RLAEKLGNKE AVVYIPVRGG RIYFATHKVA | |
| | TGAVSIVFDQ KQVWVCNADH VAAANIALTG KGIGRQSSDE | |
| | ENPDGGRIKL QLTS | |
| 453 | MSSAIKSYKS VLRPNERKNQ LLKSTIQCLE DGSAFFFKML | Variant |
| | QGLFGGITPE IVRESTEQEK QQQDIALWCA VNWFRPVSQD | Cas12i2 of |
| | SLTHTIASDN LVEKFEEYYG GTASDAIKQY FSASIGESYY | SEQ ID NO: |
| | WNDCRQQYYD LCRELGVEVS DLTHDLEILC REKCLAVATE | 496 of |
| | SNQNNSIISV LFGTGEKEDR SVKLRITKKI LEAISNLKEI | PCT/US2021/ |
| | PKNVAPIQEI ILNVAKATKE TFRQVYAGNL GAPSTLEKFI | 025257 |
| | AKDGQKEFDL KKLQTDLKKV IRGKSKERDW CCQEELRSYV | |
| | EQNTIQYDLW AWGEMFNKAH TALKIKSTRN YNFAKQRLEQ | |
| | FKEIQSLNNL LVVKKLNDFF DSEFFSGEET YTICVHHLGG | |
| | KDLSKLYKAW EDDPADPENA IVVLCDDLKN NFKKEPIRNI | |
| | LRYIFTIRQE CSAQDILAAA KYNQQLDRYK SQKANPSVLG | |
| | NQGFTWTNAV ILPEKAQRND RPNSLDLRIW LYLKLRHPDG | |
| | RWKKHHIPFY DTRFFQEIYA AGNSPVDTCQ FRTPRFGYHL | |
| | PKLTDQTAIR VNKKHVKAAK TEARIRLAIQ QGTLPVSNLK | |
| | ITEISATINS KGQVRIPVKF RVGRQKGTLQ IGDRFCGYDQ | |
| | NQTASHAYSL WEVVKEGQYH KELRCRVRFI SSGDIVSITE | |
| | NRGNQFDQLS YEGLAYPQYA DWRKKASKFV SLWQITKKNK | |
| | KKEIVTVEAK EKFDAICKYQ PRLYKENKEY AYLLRDIVRG | |
| | KSLVELQQIR QEIFRFIEQD CGVTRLGSLS LSTLETVKAV | |
| | KGIIYSYFST ALNASKNNPI SDEQRKEFDP ELFALLEKLE | |
| | LIRTRKKKQK VERIANSLIQ TCLENNIKFI RGEGDLSTIN | |
| | NATKKKANSR SMDWLARGVF NKIRQLATMH NITLFGCGSL | |
| | YTSHQDPLVH RNPDKAMKCR WAAIPVKDIG DWVLRKLSQN | |
| | LRAKNRGTGE YYHQGVKEFL SHYELQDLEE ELLKWRSDRK | |
| | SNIPCWVLQN RLAEKLGNKE AVVYIPVRGG RIYFATHKVA | |
| | TGAVSIVFDQ KQVWVCNADH VAAANIALTG KGIGRQSSDE | |
| | ENPDGGRIKL QLTS | |
| 481 | ATGGCTTCCATCTCTAGGCCATACGGCACCAAGCTGCGACCGGAC | Nucleotide |
| | GCACGGAAGAAGGAGATGCTCGATAAGTTCTTTAATACACTGACT | sequence |
| | AAGGGTCAGCGCGTGTTCGCAGACCTGGCCCTGTGCATCTATGGC | encoding |
| | TCCCTGACCCTGGAGATGGCCAAGTCTCTGGAGCCAGAAAGTGAT | Cas12i4 |
| | TCAGAACTGGTGTGCGCTATTGGGTGGTTTCGGCTGGTGGACAAG | |
| | ACCATCTGGTCCAAGGATGGCATCAAGCAGGAGAATCTGGTGAAA | |
| | CAGTACGAAGCCTATTCCGGAAAGGAGGCTTCTGAAGTGGTCAAA | |
| | ACATACCTGAACAGCCCCAGCTCCGACAAGTACGTGTGGATCGAT | |
| | TGCAGGCAGAAATTCCTGAGGTTTCAGCGCGAGCTCGGCACTCGC | |
| | AACCTGTCCGAGGACTTCGAATGTATGCTCTTTGAACAGTACATT | |
| | AGACTGACCAAGGGCGAGATCGAAGGGTATGCCGCTATTTCAAAT | |
| | ATGTTCGGAAACGGCGAGAAGGAAGACCGGAGCAAGAAAAGAATG | |
| | TACGCTACACGGATGAAAGATTGGCTGGAGGCAAACGAAAATATC | |
| | ACTTGGGAGCAGTATAGAGAGGCCCTGAAGAACCAGCTGAATGCT | |
| | AAAAACCTGGAGCAGGTTGTGGCCAATTACAAGGGGAACGCTGGC | |
| | GGGGCAGACCCCTTCTTTAAGTATAGCTTCTCCAAAGAGGGAATG | |
| | GTGAGCAAGAAAGAACATGCACAGCAGCTCGACAAGTTCAAAACC | |
| | GTCCTGAAGAACAAAGCCCGGGACCTGAATTTTCCAAACAAGGAG | |
| | AAGCTGAAGCAGTACCTGGAGGCCGAAATCGGCATTCCGGTCGAC | |
| | GCTAACGTGTACTCCCAGATGTTCTCTAACGGGGTGAGTGAGGTC | |
| | CAGCCTAAGACCACACGGAATATGTCTTTTAGTAACGAGAAACTG | |
| | GATCTGCTCACTGAACTGAAGGACCTGAACAAGGGCGATGGGTTC | |
| | GAGTACGCCAGAGAAGTGCTGAACGGGTTCTTTGACTCCGAGCTC | |
| | CACACTACCGAGGATAAGTTTAATATCACCTCTAGGTACCTGGGA | |
| | GGCGACAAATCAAACCGCCTGAGCAAACTCTATAAGATCTGGAAG | |
| | AAAGAGGGTGTGGACTGCGAGGAAGGCATTCAGCAGTTCTGTGAA | |
| | GCCGTCAAAGATAAGATGGGCCAGATCCCCATTCGAAATGTGCTG | |
| | AAGTACCTGTGGCAGTTCCGGGAGACAGTCAGTGCCGAGGATTTT | |
| | GAAGCAGCCGCTAAGGCTAACCATCTGGAGGAAAAGATCAGCCGG | |
| | GTGAAAGCCCACCCAATCGTGATTAGCAATAGGTACTGGGCTTTT | |
| | GGGACTTCCGCACTGGTGGGAAACATTATGCCCGCAGACAAGAGG | |
| | CATCAGGGAGAGTATGCCGGTCAGAATTTCAAAATGTGGCTGGAG | |
| | GCTGAACTGCACTACGATGGCAAGAAAGCAAAGCACCATCTGCCT | |
| | TTTTATAACGCCCGCTTCTTTGAGGAAGTGTACTGCTATCACCCC | |
| | TCTGTCGCCGAGATCACTCCTTTCAAAACCAAGCAGTTTGGCTGT | |
| | GAAATCGGGAAGGACATTCCAGATTACGTGAGCGTCGCTCTGAAG | |
| | GACAATCCGTATAAGAAAGCAACCAAACGAATCCTGCGTGCAATC | |
| | TACAATCCCGTCGCCAACACAACTGGCGTTGATAAGACCACAAAC | |
| | TGCAGCTTCATGATCAAACGCGAGAATGACGAATATAAGCTGGTC | |
| | ATCAACCGAAAAATTTCCGTGGATCGGCCTAAGAGAATCGAAGTG | |
| | GGCAGGACAATTATGGGGTACGACCGCAATCAGACAGCTAGCGAT | |
| | ACTTATTGGATTGGCCGGCTGGTGCCACCTGGAACCCGGGGCGCA | |
| | TACCGCATCGGAGAGTGGAGCGTCCAGTATATTAAGTCCGGGCCT | |
| | GTCCTGTCTAGTACTCAGGGAGTTAACAATTCCACTACCGACCAG | |
| | CTGGTGTACAACGGCATGCCATCAAGCTCCGAGCGGTTCAAGGCC | |
| | TGGAAGAAAGCCAGAATGGCTTTTATCCGAAAACTCATTCGTCAG | |
| | CTGAATGACGAGGGACTGGAATCTAAGGGTCAGGATTATATCCCC | |
| | GAGAACCCTTCTAGTTTCGATGTGCGGGGCGAAACCCTGTACGTC | |
| | TTTAACAGTAATTATCTGAAGGCCCTGGTGAGCAAACACAGAAAG | |
| | GCCAAGAAACCTGTTGAGGGGATCCTGGACGAGATTGAAGCCTGG | |
| | ACATCTAAAGACAAGGATTCATGCAGCCTGATGCGGCTGAGCAGC | |
| | CTGAGCGATGCTTCCATGCAGGGAATCGCCAGCCTGAAGAGTCTG | |
| | ATTAACAGCTACTTCAACAAGAATGGCTGTAAAACCATCGAGGAC | |
| | AAAGAAAAGTTTAATCCCGTGCTGTATGCCAAGCTGGTTGAGGTG | |
| | GAACAGCGGAGAACAAACAAGCGGTCTGAGAAAGTGGGAAGAATC | |
| | GCAGGTAGTCTGGAGCAGCTGGCCCTGCTGAACGGGGTTGAGGTG | |
| | GTCATCGGCGAAGCTGACCTGGGGGAGGTCGAAAAAGGAAAGAGT | |
| | AAGAAACAGAATTCACGGAACATGGATTGGTGCGCAAAGCAGGTG | |
| | GCACAGCGGCTGGAGTACAAACTGGCCTTCCATGGAATCGGTTAC | |
| | TTTGGAGTGAACCCCATGTATACCAGCCACCAGGACCCTTTCGAA | |
| | CATAGGCGCGTGGCTGATCACATCGTCATGCGAGCACGTTTTGAG | |
| | GAAGTCAACGTGGAGAACATTGCCGAATGGCACGTGCGAAATTTC | |
| | TCAAACTACCTGCGTGCAGACAGCGGCACTGGGCTGTACTATAAG | |
| | CAGGCCACCATGGACTTCCTGAAACATTACGGTCTGGAGGAACAC | |
| | GCTGAGGGCCTGGAAAATAAGAAAATCAAGTTCTATGACTTTAGA | |
| | AAGATCCTGGAGGATAAAAACCTGACAAGCGTGATCATTCCAAAG | |
| | AGGGGGGGGCGCATCTACATGGCCACCAACCCAGTGACATCCGAC | |
| | TCTACCCCGATTACATACGCCGGCAAGACTTATAATAGGTGTAAC | |
| | GCTGATGAGGTGGCAGCCGCTAATATCGTTATTTCTGTGCTGGCT | |
| | CCCCGCAGTAAGAAAAACGAGGAACAGGACGATATCCCTCTGATT | |
| | ACCAAGAAAGCCGAGAGTAAGTCACCACCGAAAGACCGGAAGAGA | |
| | TCAAAAACAAGCCAGCTGCCTCAGAAA | |
| 482 | MASISRPYGTKLRPDARKKEMLDKFFNTLTKGQRVFADLALCIYG | Cas12i4 |
| | SLTLEMAKSLEPESDSELVCAIGWFRLVDKTIWSKDGIKQENLVK | amino acid |
| | QYEAYSGKEASEVVKTYLNSPSSDKYVWIDCRQKFLRFQRELGTR | sequence |
| | NLSEDFECMLFEQYIRLTKGEIEGYAAISNMFGNGEKEDRSKKRM | |
| | YATRMKDWLEANENITWEQYREALKNQLNAKNLEQVVANYKGNAG | |
| | GADPFFKYSFSKEGMVSKKEHAQQLDKFKTVLKNKARDLNFPNKE | |
| | KLKQYLEAEIGIPVDANVYSQMFSNGVSEVQPKTTRNMSFSNEKL | |
| | DLLTELKDLNKGDGFEYAREVLNGFFDSELHTTEDKFNITSRYLG | |
| | GDKSNRLSKLYKIWKKEGVDCEEGIQQFCEAVKDKMGQIPIRNVL | |
| | KYLWQFRETVSAEDFEAAAKANHLEEKISRVKAHPIVISNRYWAF | |
| | GTSALVGNIMPADKRHQGEYAGQNFKMWLEAELHYDGKKAKHHLP | |
| | FYNARFFEEVYCYHPSVAEITPFKTKQFGCEIGKDIPDYVSVALK | |
| | DNPYKKATKRILRAIYNPVANTTGVDKTTNCSFMIKRENDEYKLV | |
| | INRKISVDRPKRIEVGRTIMGYDRNQTASDTYWIGRLVPPGTRGA | |
| | YRIGEWSVQYIKSGPVLSSTQGVNNSTTDQLVYNGMPSSSERFKA | |
| | WKKARMAFIRKLIRQLNDEGLESKGQDYIPENPSSFDVRGETLYV | |
| | FNSNYLKALVSKHRKAKKPVEGILDEIEAWTSKDKDSCSLMRLSS | |
| | LSDASMQGIASLKSLINSYFNKNGCKTIEDKEKFNPVLYAKLVEV | |
| | EQRRTNKRSEKVGRIAGSLEQLALLNGVEVVIGEADLGEVEKGKS | |
| | KKQNSRNMDWCAKQVAQRLEYKLAFHGIGYFGVNPMYTSHQDPFE | |
| | HRRVADHIVMRARFEEVNVENIAEWHVRNFSNYLRADSGTGLYYK | |
| | QATMDFLKHYGLEEHAEGLENKKIKFYDFRKILEDKNLTSVIIPK | |
| | RGGRIYMATNPVTSDSTPITYAGKTYNRCNADEVAAANIVISVLA | |
| | PRSKKNEEQDDIPLITKKAESKSPPKDRKRSKTSQLPQK | |
| 483 | MASISRPYGT KLRPDARKKE MLDKFFNTLT KGQRVFADLA | Variant |
| | LCIYGSLTLE MAKSLEPESD SELVCAIGWF RLVDKTIWSK | Cas12i4 A |
| | DGIKQENLVK QYEAYSGKEA SEVVKTYLNS PSSDKYVWID | |
| | CRQKFLRFQR ELGTRNLSED FECMLFEQYI RLTKGEIEGY | |
| | AAISNMFGNG EKEDRSKKRM YATRMKDWLE ANENITWEQY | |
| | REALKNQLNA KNLEQVVANY KGNAGGADPF FKYSFSKEGM | |
| | VSKKEHAQQL DKFKTVLKNK ARDLNFPNKE KLKQYLEAEI | |
| | GIPVDANVYS QMFSNGVSEV QPKTTRNMSF SNEKLDLLTE | |
| | LKDLNKGDGF EYAREVLNGF FDSELHTTED KFNITSRYLG | |
| | GDKSNRLSKL YKIWKKEGVD CEEGIQQFCE AVKDKMGQIP | |
| | IRNVLKYLWQ FRETVSAEDF EAAAKANHLE EKISRVKAHP | |
| | IVISNRYWAF GTSALVGNIM PADKRHQGEY AGQNFKMWLE | |
| | AELHYDGKKA KHHLPFYNAR FFEEVYCYHP SVAEITPFKT | |
| | KQFGCEIGKD IPDYVSVALK DNPYKKATKR ILRAIYNPVA | |
| | NTTGVDKTTN CSFMIKREND EYKLVINRKI SRDRPKRIEV | |
| | GRTIMGYDRN QTASDTYWIG RLVPPGTRGA YRIGEWSVQY | |
| | IKSGPVLSST QGVNNSTTDQ LVYNGMPSSS ERFKAWKKAR | |
| | MAFIRKLIRQ LNDEGLESKG QDYIPENPSS FDVRGETLYV | |
| | FNSNYLKALV SKHRKAKKPV EGILDEIEAW TSKDKDSCSL | |
| | MRLSSLSDAS MQGIASLKSL INSYFNKNGC KTIEDKEKEN | |
| | PVLYAKLVEV EQRRINKRSE KVGRIAGSLE QLALLNGVEV | |
| | VIGEADLGEV EKGKSKKQNS RNMDWCAKQV AQRLEYKLAF | |
| | HGIGYFGVNP MYTSHQDPFE HRRVADHIVM RARFEEVNVE | |
| | NIAEWHVRNF SNYLRADSGT GLYYKQATMD FLKHYGLEEH | |
| | AEGLENKKIK FYDFRKILED KNLTSVIIPK RGGRIYMATN | |
| | PVTSDSTPIT YAGKTYNRCN ADEVAAANIV ISVLAPRSKK | |
| | NREQDDIPLI TKKAESKSPP KDRKRSKTSQ LPQK | |
| 484 | MASISRPYGT KLRPDARKKE MLDKFFNTLT KGQRVFADLA | Variant |
| | LCIYGSLTLE MAKSLEPESD SELVCAIGWF RLVDKTIWSK | Cas12i4 B |
| | DGIKQENLVK QYEAYSGKEA SEVVKTYLNS PSSDKYVWID | |
| | CRQKFLRFQR ELGTRNLSED FECMLFEQYI RLTKGEIEGY | |
| | AAISNMFGNG EKEDRSKKRM YATRMKDWLE ANENITWEQY | |
| | REALKNQLNA KNLEQVVANY KGNAGGADPF FKYSFSKEGM | |
| | VSKKEHAQQL DKFKTVLKNK ARDLNFPNKE KLKQYLEAEI | |
| | GIPVDANVYS QMFSNGVSEV QPKTTRNMSF SNEKLDLLTE | |
| | LKDLNKGDGF EYAREVLNGF FDSELHTTED KFNITSRYLG | |
| | GDKSNRLSKL YKIWKKEGVD CEEGIQQFCE AVKDKMGQIP | |
| | IRNVLKYLWQ FRETVSAEDF EAAAKANHLE EKISRVKAHP | |
| | IVISNRYWAF GTSALVGNIM PADKRHQGEY AGQNFKMWLR | |
| | AELHYDGKKA KHHLPFYNAR FFEEVYCYHP SVAEITPFKT | |
| | KQFGCEIGKD IPDYVSVALK DNPYKKATKR ILRAIYNPVA | |
| | NTTRVDKTTN CSFMIKREND EYKLVINRKI SRDRPKRIEV | |
| | GRTIMGYDRN QTASDTYWIG RLVPPGTRGA YRIGEWSVQY | |
| | IKSGPVLSST QGVNNSTTDQ LVYNGMPSSS ERFKAWKKAR | |
| | MAFIRKLIRQ LNDEGLESKG QDYIPENPSS FDVRGETLYV | |
| | FNSNYLKALV SKHRKAKKPV EGILDEIEAW TSKDKDSCSL | |
| | MRLSSLSDAS MQGIASLKSL INSYFNKNGC KTIEDKEKEN | |
| | PVLYAKLVEV EQRRTNKRSE KVGRIAGSLE QLALLNGVEV | |
| | VIGEADLGEV EKGKSKKQNS RNMDWCAKQV AQRLEYKLAF | |
| | HGIGYFGVNP MYTSHQDPFE HRRVADHIVM RARFEEVNVE | |
| | NIAEWHVRNF SNYLRADSGT GLYYKQATMD FLKHYGLEEH | |
| | AEGLENKKIK FYDFRKILED KNLTSVIIPK RGGRIYMATN | |
| | PVTSDSTPIT YAGKTYNRCN ADEVAAANIV ISVLAPRSKK | |
| | NREQDDIPLI TKKAESKSPP KDRKRSKTSQ LPQK | |
| 4503 | MSNKEKNASETRKAYTTKMIPRSHDRMKLLGNFMDYLMDGTPIFF | Cas12i1 |
| | ELWNQFGGGIDRDIISGTANKDKISDDLLLAVNWFKVMPINSKPQ | (SEQ ID |
| | GVSPSNLANLFQQYSGSEPDIQAQEYFASNFDTEKHQWKDMRVEY | NO: 3 of |
| | ERLLAELQLSRSDMHHDLKLMYKEKCIGLSLSTAHYITSVMFGTG | U.S. Pat. |
| | AKNNRQTKHQFYSKVIQLLEESTQINSVEQLASIILKAGDCDSYR | No. |
| | KLRIRCSRKGATPSILKIVQDYELGTNHDDEVNVPSLIANLKEKL | 10,808,245) |
| | GRFEYECEWKCMEKIKAFLASKVGPYYLGSYSAMLENALSPIKGM | |
| | TTKNCKFVLKQIDAKNDIKYENEPFGKIVEGFFDSPYFESDTNVK | |
| | WVLHPHHIGESNIKTLWEDLNAIHSKYEEDIASLSEDKKEKRIKV | |
| | YQGDVCQTINTYCEEVGKEAKTPLVQLLRYLYSRKDDIAVDKIID | |
| | GITFLSKKHKVEKQKINPVIQKYPSFNFGNNSKLLGKIISPKDKL | |
| | KHNLKCNRNQVDNYIWIEIKVLNTKTMRWEKHHYALSSTRFLEEV | |
| | YYPATSENPPDALAARFRTKTNGYEGKPALSAEQIEQIRSAPVGL | |
| | RKVKKRQMRLEAARQQNLLPRYTWGKDFNINICKRGNNFEVTLAT | |
| | KVKKKKEKNYKVVLGYDANIVRKNTYAAIEAHANGDGVIDYNDLP | |
| | VKPIESGFVTVESQVRDKSYDQLSYNGVKLLYCKPHVESRRSFLE | |
| | KYRNGTMKDNRGNNIQIDFMKDFEAIADDETSLYYFNMKYCKLLQ | |
| | SSIRNHSSQAKEYREEIFELLRDGKLSVLKLSSLSNLSFVMFKVA | |
| | KSLIGTYFGHLLKKPKNSKSDVKAPPITDEDKQKADPEMFALRLA | |
| | LEEKRLNKVKSKKEVIANKIVAKALELRDKYGPVLIKGENISDTT | |
| | KKGKKSSTNSFLMDWLARGVANKVKEMVMMHQGLEFVEVNPNFTS | |
| | HQDPFVHKNPENTFRARYSRCTPSELTEKNRKEILSFLSDKPSKR | |
| | PTNAYYNEGAMAFLATYGLKKNDVLGVSLEKFKQIMANILHQRSE | |
| | DQLLFPSRGGMFYLATYKLDADATSVNWNGKQFWVCNADLVAAYN | |
| | VGLVDIQKDFKKK | |
| 4504 | MSISNNNILPYNPKLLPDDRKHKMLVDTFNQLDLIRNNLHDMIIA | Cas12i3 |
| | LYGALKYDNIKQFASKEKPHISADALCSINWFRLVKTNERKPAIE | (SEQ ID |
| | SNQIISKFIQYSGHTPDKYALSHITGNHEPSHKWIDCREYAINYA | NO: 14 of |
| | RIMHLSFSQFQDLATACLNCKILILNGTLTSSWAWGANSALFGGS | U.S. Pat. |
| | DKENFSVKAKILNSFIENLKDEMNTTKFQVVEKVCQQIGSSDAAD | No. |
| | LFDLYRSTVKDGNRGPATGRNPKVMNLFSQDGEISSEQREDFIES | 10,808,245) |
| | FQKVMQEKNSKQIIPHLDKLKYHLVKQSGLYDIYSWAAAIKNANS | |
| | TIVASNSSNLNTILNKTEKQQTFEELRKDEKIVACSKILLSVNDT | |
| | LPEDLHYNPSTSNLGKNLDVFFDLLNENSVHTIENKEEKNKIVKE | |
| | CVNQYMEECKGLNKPPMPVLLTFISDYAHKHQAQDFLSAAKMNFI | |
| | DLKIKSIKVVPTVHGSSPYTWISNLSKKNKDGKMIRTPNSSLIGW | |
| | IIPPEEIHDQKFAGQNPIIWAVLRVYCNNKWEMHHFPFSDSRFFT | |
| | EVYAYKPNLPYLPGGENRSKRFGYRHSTNLSNESRQILLDKSKYA | |
| | KANKSVLRCMENMTHNVVFDPKTSLNIRIKTDKNNSPVLDDKGRI | |
| | TFVMQINHRILEKYNNTKIEIGDRILAYDQNQSENHTYAILQRTE | |
| | EGSHAHQFNGWYVRVLETGKVTSIVQGLSGPIDQLNYDGMPVTSH | |
| | KFNCWQADRSAFVSQFASLKISETETFDEAYQAINAQGAYTWNLF | |
| | YLRILRKALRVCHMENINQFREEILAISKNRLSPMSLGSLSQNSL | |
| | KMIRAFKSIINCYMSRMSFVDELQKKEGDLELHTIMRLTDNKLND | |
| | KRVEKINRASSFLINKAHSMGCKMIVGESDLPVADSKTSKKQNVD | |
| | RMDWCARALSHKVEYACKLMGLAYRGIPAYMSSHQDPLVHLVESK | |
| | RSVLRPRFVVADKSDVKQHHLDNLRRMLNSKTKVGTAVYYREAVE | |
| | LMCEELGIHKTDMAKGKVSLSDFVDKFIGEKAIFPQRGGRFYMST | |
| | KRLTTGAKLICYSGSDVWLSDADEIAAINIGMFVVCDQTGAFKKK | |
| | KKEKLDDEECDILPFRPM | |
| 454 | AGTCTTCTCTCTCGCTCTCTCCGCTGCTGTAGCCGGACCCTTTGC | STMN2 |
| | CTTCGCCACTGCTCAGCGTCTGCACATCCCTACAATGGCTAAAAC | |
| | AGCAATGGGTAAGGCACTGCGCCTCGTTCTCCGTCGGCTCTACCT | |
| | GGAGCCCACCTCTCACCTCCTCTCTTGAGCTCTAGAAGCATTCAG | |
| | AGATATTTTATAAAGAAAAAGATGTTAATGGTAACACAGGACCAG | |
| | GAAGGACAGGGCAGTTCTGGGGGAGGTGGGAGGGCAGAGAAGAGG | |
| | TCTATGGAAATCTAAAGCGAAGAATTTCTTTTAAAAGGTAGAAGC | |
| | GGGTAAGTTGCCCTCCTATGGGTAGAGAATTTATTCTGTTTCCAT | |
| | ATTTAAAATTAGGACTCAATCGTGAGGGGAGGAAGCTACCTTAAC | |
| | TGTTTGCCTTAAATGGGCTTAAGGGACATTTTGGAAAGTGCTTTA | |
| | TAACGACCTTTTTTTTTTTTATTTCTTCTCTAGTTTAAGAAGAAA | |
| | ATAGGAAAGGGGTAAAGGGAAGGTGGGAGAAAGGAAAAAGAAAAT | |
| | TGCAAAGTCAAAGCGGTCCCATCCCGCTGTTTGAAAGATGGGTGG | |
| | AGACGGGGGGAGGGGATGGAGAGAACTGGGCACATTTTACGGTAT | |
| | TGTCTCGTCGAAGAAACCGCTAGTCCTGGGGTGCGGTGCAGGGAG | |
| | GTAAGACGGCGGGGGACAGGGTGGGGGTAGGACCTCCGCTCCTTT | |
| | GTTTTAGGGCAAGGGAGGGGAAGGAGAGAGGAAGTCGCGGAGGGC | |
| | GTGGAGGGCGCGGGTGGGCAGCTGCAGGGGGGGGGAAGCGCGCGG | |
| | CAGGGAGGGGTGGAGGGACAGCGGCTTCGAAGGCGCTGGGGTGGG | |
| | GTTTCTTTGTGTGCGGACCAGCGGTCCCGGGGGGAGGCACCTGCA | |
| | GCGCTGGGCGCACAATGCGGACAGCCCCACCCAGTGCGGAACCGC | |
| | GCAGCCCCGCCCCCCCGCCCGGTGCTGCATCTTCATTCGAAAGGG | |
| | GGTCGGGTGGGGAGCGCAGCGTGACACCCAGGAGCCCAACCCTGC | |
| | GGGGACAGCGGCGCCACGCCCCGCGCTCCCCGCTCCCGACTCCCC | |
| | GCCGCGGCTTCCAAGAGAGACCTGACCACTGACCCCGCCCTCCCC | |
| | ACGCTGGCCTCATTGTTCTGCTTTTAAGAGAGATGGGAAAAGTGG | |
| | GTTAACATTTTTCTTTTCGGAAGCAAATTACATAGAGTGTTTAGA | |
| | CATAGACACAGATAAAGGGTTCTTTGAAGACCTTTGATCGTTTGC | |
| | GGGAAAAGCTTCTAGAACCTAGACATGTGTATGTATAATAATAGA | |
| | GATGACATGAAATCGTATATAAAGCAAAAGAGGTCAAAGTCTTAA | |
| | GTTAAGCCACGCGAAATTTCCGTTTTGTGGGTCAGACAGTGCCAA | |
| | ATATCGGCAATTTCATAAGCTCAGAGAGACAAGACAGTGGAGACA | |
| | CAGGATGACCGGAAAAGATTCTGGATTCAGGGCCTTCATCCGCAA | |
| | TTGGTCTTGTGCCTTGAGTGCCCACGGTTCTGGCGCTCAGTGGCC | |
| | CCGGGGTGAAAAGGCAGGGTGGGGCCTGGGGTCCTGTGGCAGCTG | |
| | GAAGCACGTGTCCCCCGGGACTTGGTTGCAGGATGCGGAGACAGG | |
| | GAAAGCTGCCGAAAGGACTCCATCTGCGCGGCTCCGCCCTGCCCT | |
| | ACCCTCCCCGCGGAGCCGGGGAGACCTCAGGCTCCGAGACTGGCG | |
| | GGGAAGAGGAATATGGGAGGGGCAGTTGAGCTGTATGCAGTCCTG | |
| | GAACCTCTTTTTTCAGCCCCGCAGTCCACAACGGCCCGAGCACCC | |
| | CTTGATGTGCGCAGACCCCCGGCGTGGCTCTCAGCCCCAGCACCG | |
| | AGCCCCTCCCAGCCAAGCGGGTGGCTCTGCAGAAAAGCTGGCTCG | |
| | AGCCCCGCCCGGCCACACAAAGGCGCGGCCCCACCCAGCCCGGGC | |
| | GCGAGACCGCAGAGGTGACCCCCTTCCCAGGGATTCAGGGAGGGC | |
| | TGTCTCTTCTCGCCCACCCACGGTCCGCGGAGCTCGGGGCTTTTT | |
| | TTCCCCCAGCCCAAGCCCCCCGCCCACCCTCTGTTCTCTATGATT | |
| | TTCCAGAATGGAGACCCCGCGAGGGGCTTCTCTAAGGGAGACCCT | |
| | CGCTCCTCCAGCGGGGCGCGGCTCGGCCCCACCCCTCCCAGCTGA | |
| | GGCCCAGAGCCGCCTACCGCTGGCCGGGTGGGGGCGCACGTGGCG | |
| | ACTGGGTGTGTGGAGCGCAGCCAGCCCTGCAGAGCCCCGCGCCGC | |
| | GCCCTGCGCTCCCCTCCCCGGAGTTGGGCGCTCGCCCCCGCGGTG | |
| | CAGCCGGGGAGACCGGTTTCTGCGCAGTGTCCTGAGCTACCCCCG | |
| | CTTTCCACAATTCGCAGTTCACTCGCACGTCCAGAAAGGTTCTGA | |
| | GAATGGGTGGTGGGGGCGATCTCGCCTCGCTTTCTGCACCCCTCA | |
| | GAAAGGTTTCCGCTGCAGGCTAGTGGCTGCAAACTCATCGTCATC | |
| | ATCAGTATTATTATCATTTCAAATCGTTGTTATTATTTAATGATT | |
| | CAGTAGCCTTGTTTGTTCTCATTTGTTCAAAAGGGACGTGGATTG | |
| | CTCTTGGTTAAGGATTAACCCTTGTTGCGTTCGCTTTGCTTCCTC | |
| | CTAATTGCCCTCATCCCTTTCCCCCACAAAAAGGTAAATTTGTCT | |
| | CCAGTTGTTCATTTTAAGTTATAAAGCAAATATATTTTTGCTTCC | |
| | TGCCAGGATTATGTATGTTCATGTGGCTAAGATACATGTGCAAGT | |
| | GCTTGCTAAGAGCAGGGTTTGTGTGCCAACGATTGCTGGAAAATT | |
| | CTCTGCAAAGAATTGTTTGTGGCTGCAATGGGTGAGAATACACAT | |
| | ATATAATTGAGATGATCTTCAACATAAGGTTATATCTATAAATAT | |
| | ATAAATATAGTTTATGCACAAAATTTTAAGTTTTTTCCCCTGAAA | |
| | CTGTTCTTCCAACTGCTGATTCTTGATACAGCCTCAATCCTACAC | |
| | AGATACATGGATCGTGAAATGGTAGCCGCCATCCAAATAAAAATC | |
| | CCACCCCAAATATGACAAACGCAAGCATCCTTTCTGGCCATAATT | |
| | TAACTGCATTTGCAAATCATGAAAAAAACACTACTTCTGCAGTAT | |
| | TAAAATAATAGATTTTGAAATTAATTCCAATTTCAAAGATAATTA | |
| | ATTATCAGGGCGAGTGCTTTTTTCCTGATTCATTAAACAATTATG | |
| | TATTCAGCATGATTGTAAGAGGTGCATATAATATTCCCCATTATC | |
| | TTTTCTAATGAAGTGGGCACCTTCTGAATGGATATATAAGTAACT | |
| | AGAAATGAAAAGCTGAGGATTTGGTCAGAATTTCAGGATAAAACT | |
| | GAAAGAAATGGCAGTAGTTTATCAATTAATCTCATGTATTTAGTT | |
| | TATACCAGGTGAGTAAGCTGAGCCTGCAATAAACACTCTCTGTCC | |
| | CAGTGTAACACGTCGCAGGTAGCTAGAATGATAGGATAAATTAAT | |
| | AGACCTTGTGGTGTTTGTCTATGCACGTTAAAATTCTCTGAGAGA | |
| | AAGTATATTTTAAAATGATAATTAAGATTGGACATTTGTGCTATT | |
| | AAAATCTACAACTTTAGTCAAAATTCACAATGGTTTTTTTTTACA | |
| | ATAATGTGACTTACAGATTTGTAGTAAATTATTCTATTCTAAAAG | |
| | AGAAATGAGTGTTTTTATTGTTACAGCTATTACCTCATTAATATT | |
| | TTTAGCAAACTTTTATTTGTTGCATTGAAAGCAGTTTTAATTACT | |
| | TTGGGTTTTTATTTTTCAAATTACTAATGGATAGATGGTGGAATA | |
| | AGCATTTAATCATTTGGCACAATATGACTTCCATCAAATAGCTCA | |
| | TTCTCAGTGATTAAAAAATGCTACAAGAGGCTACAATTTACTCAG | |
| | ATTCAGGAAATGTCCTTTCAGAGTGCCATAAGGCTGATTCATATA | |
| | ATAAAATAGTTTTCTTCCCTATAATTTAAGATCAAATAGTTACTT | |
| | AGTTCTGTGAATACCTAGCAGTAGCTATCAAACAGAATTTTAAAG | |
| | TTAAATCTGTACAACTAACAATGAAGTGGAGGATGAATCGATACA | |
| | TATTGAATGGAAGACTTTGTCATTGATAAATTCAGGCCATCTTTA | |
| | GGAAAATTCCGGATTTATCAATCACCATTATTTTTTACTTCAACT | |
| | GAGTGTGACTGATCACATGCTCAGGCTACCTTGGTAGCTCATTGC | |
| | TCACAGGAGGCTGAAAAAAGCTGGCCTCCGAGCAGGAGGAAGCTC | |
| | AGAGCACAAACCTAGGCCTGGGCGTGGCCACTGGGAGCTGCTGAT | |
| | AGCGAACCCCAGCTCACACCAGTTTCTTTTTTGGTCGTGGGAAGA | |
| | AAAACACATATTATCCTGTTGTCACAAGATCTGTGACCTTATATG | |
| | AAAAAATGCTAGAATTTTTTCATTAAAAAAGAAAATACTGAACTA | |
| | GCCAGTGACCCAGATGTTTTCAGAACCTAGACTGGTTCTGTCCAT | |
| | TGGAAAACCTCGGTGTCTGCATTAACTTTTCACCACACTAGAGGG | |
| | CAATCATGTTCTCTAAAAAAGCAGATGATTGATGTAAACCTAGTT | |
| | CCAAATATTAACTGTTTAATAAAATCTTTTCTTTTACCAGGAACA | |
| | TTCAAGTGTTTATTCAATAAGCTGATGCCATGCTTTACCCTAGTG | |
| | GATGAACAGAGCTTGTACAATTTTCAAGGAGACAGGATGAAATGA | |
| | GTGGTCATAATCTGAAAGTAGATACACGCCCTGGTTAATTATTCC | |
| | CTGATGGTTTTACTTCTCAGTTTTATTACATTGTTATTATAATAC | |
| | CATTTATGTTACTTCTGAGATTTTGTAGTGGATAAATAGTAGAAA | |
| | AATGTCAGTAGTAATAGCAAAGTTATTTAGCAGCCGAATATTTTA | |
| | ATGCTTAAAAATAAAGGAATAAATTAAAGAAAATCATTGTTTACT | |
| | TCTTCATCGATTGAAATGTGCCCCCTGTTCAGAGCACATCTGAAT | |
| | ATCAGAGTCTCCACCTGCAGAGAACATGCAGCTTAGCGAGTAAAA | |
| | CAGGCAGGTATGTGATACTGAGGAGGTGTACCAAAAACTGACTGC | |
| | TGTTATTTTTCCCATCTTCTAAGTCTGTCTTTCTTTTCCATTTAA | |
| | AGATACCTTTTTAAATCTAATCCAATGTGATTTCAATCTAGTTTT | |
| | ATCAGATTTCAACAATTATTGAGCATCTCCTTGTAGTGGTTTTCT | |
| | GTTTATTAGAAAATCGATGTTAATTTTAACGAAGTAAGAAGAAAT | |
| | ATATAAGTATAAACTAATTTTGGGTATCATCAAAAGTGGATTTTT | |
| | TAAATATGCATTGATAGAATTATTTTTTGATTACATTTTATGTAA | |
| | TTCTAATCCAGCTATAAAATATTTAATAGTGTCATATTACTGTGT | |
| | TCCTCAAACTTTGATGTGCATATGAATTACCTTTGATTTTCATTA | |
| | AAATGCAAATTCTGATTCAATACATCTGGCTTGAGGCAGACATTC | |
| | TGTCTTCCGAACAAGCTCCCAGATGATGCTGATTCTGACCACTAA | |
| | ACACATCAGTTTTAGGGATATTAACTTGTAATATACAGGTATCCC | |
| | TCCTGGTAAGCTCTGGTATTATGTCTTAACATTTTTAAATCTATG | |
| | GTAATCTTTACAAAATATTTTACTTCCGAACTCATATACCTGGGG | |
| | ATTTTATTACTCTGGGAATTATGTGTTCTGCCCCATCACTCTCTC | |
| | TTAATTGGATTTTTAAAATTATATTCATATTGCAGGACTCGGCAG | |
| | AAGACCTTCGAGAGAAAGGTAGAAAATAAGAATTTGGCTCTCTGT | |
| | GTGAGCATGTGTGCGTGTGTGCGAGAGAGAGAGACAGACAGCCTG | |
| | CCTAAGAAGAAATGAATGTGAATGCGGCTTGTGGCACAGTTGACA | |
| | AGGATGATAAATCAATAATGCAAGCTTACTATCATTTATGAATAG | |
| | CAATACTGAAGAAATTAAAACAAAAGATTGCTGTCTCAATATATC | |
| | TTATATTTATTATTTACCAAATTATTCTAAGAGTATTTCTTCCTG | |
| | AATACCATGTGAGAAAATTCTTAAGAATTTATTGAGTATGACTGT | |
| | ATATTTGAAAAGAGTGTTTTCTTCTGCTTATCTAAGCCAATAAAG | |
| | GATCTTCATTATTCAATTCTAACTTTCTAAGGAAGTCAACCTACA | |
| | GATCAGAAAGAGGATCTTCAAGGAATAGCATCAAAGACATAGTCA | |
| | GGTCTCCCATGCAGTGACTGGCTGACCATGCAGCCATTACCACCT | |
| | TTCTGGAAATATTATGCTGCAAAAATGATACAATACACGAAATAT | |
| | CTCAAATTAAAAAATATAACATTTCCCAAATAGGGCACTAAAAAC | |
| | ATGATCCCAAATAAAACTAGCTTCAGGGTTTGCAGAATATACTGT | |
| | TACTCAACACAAAGTTGGACTAAGTCTCAAAGTTAGCCATTCAGT | |
| | TGTTGTTAACAGTTCATTTCAGGGTCTCTCAGAAGCTGGGAAACT | |
| | TTCCATTTTTGCAATTTCTTGTACATTGAAGGAAAGGAAGACACA | |
| | CTTAAGACAGCATTACAAAAGTAATTCATGTTTTAAATGTTTAAT | |
| | TCTGGCAGTCGGGCAGGGCTCTCTGTATAACCTCATTTGGAGATG | |
| | ACAAAAATCTAAACTTGAGGGCCTCGAGCCAATAAGTCTTCCTAT | |
| | TTCTTTACTCAAACATTTTCCCGCAATGGTGCTTTCTTTCAACTG | |
| | TTTTTCTGGTGTATTCATAAATTCCAGATTCTCTATGGGAAGTAA | |
| | CTTTTATTGATTGATTTAACCCTTGTATAGCACATATAACATGCA | |
| | AGGCATTGTTCTAAGAACTTTCCACATATTAACTGTGTTAATCAC | |
| | TTAATAATCCTAAGTAGGTTCTATTACAGATATGGAAACTGAGGC | |
| | ACAGAAAGTTGAAGTATCTTACTCAAGGTCACACAGTTAGTCAGA | |
| | TCCAGAATTTGGGCCCAGGCCATCTGGCTTCGGAATCCATCTTTC | |
| | ACCGATTGCTGCTAGTCTCATATCTGTTCCATGTTAGAGGTGAGC | |
| | TCCCATTGCAGAGGTCACACCTGTGATATCACCATTTTATTTAAA | |
| | CAGACCAGAGATGGTCTTCTCCTTTCTGATCACAGACTCACCTTG | |
| | AAGAGAAAATACTTCCAAATTGATGCCTAGTTTTAATAGCTTACC | |
| | TGGGGCTTATTCAAATAATTGCCATGATTTAGGCTTTGGGAGAAA | |
| | GAGAGCTATGAGGCCGTGTGGGTTGTAACGTATGAGACACATGGC | |
| | GTTCTGCAGGCTCAGCACAGCATCGATTTCTGGTGGGAACACACT | |
| | CTGATGACCAGTTCCAGAAATAACATTGACTTAATCTCCTCAGTC | |
| | CCATCATGGTTAGCACATTTCAAAATGCCTCCTTAACTACTTCCA | |
| | TAGGCCAGAGATATTTAGTTTTAACATTTTGTTGAATAAAATAAA | |
| | TTTACACATTCACATTTAATATAACTATTAGATGTTATTTCAAGA | |
| | TTCTCTTCATATTACCATCAAAGCAGGCAGGCAGGCAGGAGAGAA | |
| | CTGTAGGAAGGTTTTGAATCCCTTGTGAAACATTTTTAATTATCT | |
| | TTTAATAAAGGAATCAGGCCCTGTCATTTGTCAAGGAGACATTTG | |
| | CAGTAGTAAAGCTTGTGTTTATAATATCCATTTTTATTAGTCATG | |
| | ATTAAAGATAACATTTGTGTACATTTGTTCTCACAAAACACTTTT | |
| | ATATGAGTGTAAAGGTTAATTAATGCATTTCAGCCATCATTTTGC | |
| | TGGTCATGTGGAAATATAGCTTCTTTAGGAATTGTACTTAGAGTA | |
| | GGAGCCACATATTATACTATAAAACCATAACAAAAATATTTTAAG | |
| | TTTGTTCTCACTTGTTGTTGACCTCCAGAGTAAAATATTTAATAC | |
| | TCTGGAAAGTTATGGGTTTCAAAATTTATTTTATGGCAAGAAATA | |
| | GATAATTACAGTTCTCATAGAGCACATTTAAAATAATTTATTTTT | |
| | ATAGGGCAAAAATATTGCCTAGGACTGAATGATTTTTTTTTTTTT | |
| | ACAAAGATTGTAAAGCAACGCCTGCAAGAGTGCCCATTTAGCAGT | |
| | TATTCTTCTGGAATAATTGTATTTTGGATGTTGGAGTTCGCACAT | |
| | TAACCATTAGTACAAGTACCCAATATAACAATAGATCATCAGGAT | |
| | AATAAATCTGTCCATCTTTTAGTTGTATGTCTTTATATCAGGATA | |
| | AAGAGAATTGAGTGAAATTTATCTAAACCTAGTCCCACAAATACT | |
| | TTTACAAGAGAGCATGTTAAAGTGTAAATTAAATTTTTATTAGCA | |
| | TTCTACTCTGTCTTTGGAAGTTTTTTTTCCTTATGAAATGCAGCC | |
| | ATAAAGTTTAACTTCCATTAACAAAGCTGCTCACAGTAAACCTAT | |
| | TATAATAATAGTTTCCCAGTTTGGGCTTCCTAGTGAGGAGCAACC | |
| | TAACTCACACGAAACAACCCCAACTTATAATATATTGACTGTTAC | |
| | AAAACTGAGACCAGAAAATCCCATCAAGATGGTACTGTTATCATT | |
| | TCCAGACTCTCGGGAAGAACATTAATCATCTCAGGCACTTTTAGG | |
| | ATAGACTTATTGCAGCCTCCCTGGGAACTCTGCTTCAGAACATAA | |
| | TTATTTTTATTAATGCAGAGTTACTTTTTATTTCCAACAAAAATA | |
| | TCTATTGTTATTATTTAAGTCTTACAGCTTTATCTGAGAAATTCC | |
| | AATTAGCACCCTTCTCATAATAAATATTCAAACACATGAAAAATT | |
| | ACCAAAGTTGTTCTAGTCTTTTAATGACATATTACATGATCCTGC | |
| | ACTCTTGTCACTTTAAAAATTATCTTTTTATTATATTTCTGATGA | |
| | TTTTTTTCTTATATAGTTTTTTAAAAGGAGCAGGCAAGCATAGAA | |
| | GACTAAAAAATGTTCAAAAGAAAAATTAAATCGCATGATCTATCT | |
| | ATATGGGACCTTGTCATTTTTAGAAAACATTCACCTGCTTCATCC | |
| | TTTTGAATCTTCATATAATCCCTCTGAGATGGGCATACTATACAA | |
| | GTTGTCTTATTTAAAGATTGGTAAATTTAAGCTCAAATAATTTAT | |
| | TCAGTGGCAAGCCTCAGAGGCAGACTCGGAACACAGGTCTAATAT | |
| | ATATTATATATATATTATAACATATAATATATATATTACATATAA | |
| | TAAAGTTGTGTATATTATTTACCTATCAAAATATTTATATGTAAT | |
| | ATATAAATATGTTATATATCATGTATGTGCCTATTTCATACATAT | |
| | ATACACATTCATGCAAAATAAGGTTTAGCACTCCCTCCACTGTCC | |
| | TGTAATAAAACATGCACAGTGAGAATAGTCATACACGAGGCATAT | |
| | TTGTCTTCAGTTTAAAGTCATTGATAGTCAGTGTCACTAACTAAA | |
| | GTAAAATAGATTGGAGCACCAACTTTGTTCTGAAGCCTGTGCCAG | |
| | GTATTATGAGAACAAAAATAAAAATGTTCCTCACCCTTGGTGGAT | |
| | TTAGTCTTTTGCAGAAAAAAAGATCCTGTACATGTCAGAAAGTTC | |
| | AATAGTAATAATGGTAATTTATAACTATAAATGGAAGTCACCATC | |
| | TCACAATTTCACCATCTTAACAATTTTGTTAAACTGCCCTACAAT | |
| | ATTACAAGATAGTACATAATGATACACTAGTAACATCAACTAGGA | |
| | AGTACCAAGATCCACCAAAAGGCTGAAAAATTTAAATATTTAATG | |
| | AGTCCATCAACCAATCTGGCCAGAGAATTCTTTAATTAAAATGCT | |
| | TCCCAAATTTTACTGAGAATCAGCAGCGTTTGAGGAGCTAGCCTC | |
| | CACCCCCAGAGGTTCTCACTCTATTAGGTCTGAAGCAGGTCCCAT | |
| | GGATTTGCATTTCTAACAAGCTCCCAGGTGGTGCTGATGAGGCTG | |
| | ATTCAGAACCACACTTGGAGTAGACCTAAAACAGCAGTGACCTGT | |
| | AGGGTCCCCAAGCAGCAGGCCAGGACAGCATGTGAGTTACGTCCT | |
| | CTGTGGAGCTCTGCAACAAGGCGTCAAGAGGTCAGAGTCTAAGTC | |
| | CCCATCAGCTCTGCCCTTCTCCACCAGTGCTGCTGGTGCTGCATG | |
| | GAAGGAAGAGCCCAGAAGGGATTCTGAGTTTCAGTCTTTACTCTT | |
| | GCTGACGCACCTTGGTCAGGTCAATTTTCCTGTTTGTTCCTCTAA | |
| | TTCAGCATCTGTAAAATAGCCATGTGAACTGCCTTGTCCATATCA | |
| | GAGGGTCTTTTTCAGACTCAAGGAAAAAAACGTGAAAGTGATTAG | |
| | TGTCTGTCAAGTAGTATATAAATGCAAGAAGTTGAGTTTTTAAAT | |
| | TGTCATTAGATATAAATACCCATGTGCATGCATTTAGAATGAGTA | |
| | AAGAGGGAACAAGGAGCGCAATCAAAAACTGCGTCATTTGCTTTT | |
| | TGAAAAATACTTTCTATGTAATGAAAAGTGAAATAAAATGTTAAT | |
| | TGAGTCCCTCTGACAACAGCATCAGACGTTTTGCAGTTCTTGTGA | |
| | TTAGAACCCACCTGGCCAGCCCTTCTTCCTCCTAAAGAAGAGCCT | |
| | TCTTCTTCTTAAATGAAGGTTGGCTCAGAAGAAGCAATTAACTCA | |
| | TTCAACGTTTTGTTACAGTCAATCCACATCCAACTTTTCCCCAAC | |
| | TCAATCTGCTTTAAGGGAAGGATGGTAAGTGGTGGCCCAAGATGG | |
| | CAACCATCAAGCTTAGAGAATCTCTAGAAGCAGGGGTGTCCCCAG | |
| | CAAGTAGACACTGAAAATATGAGAGGGCTGATAAGCCAGAGATAA | |
| | AACTCAGTACTTACTTTGCTTCTAGTCCATGTCTACCCCTTTCTT | |
| | GGCACCACCTTGACACTACCCTCTGAGTCCACCTTCCTGAGATGG | |
| | TACAAACTCTGCTTAGACAAAGCAGCCCATGTCCAAAGGTGTTAG | |
| | GGCTCAGTTTAAAGCTGCCTTCAAAAGTTAAAACAGAAGTGTAAA | |
| | GTTCTGTGCAATTAAAAATAATCAGCTTGTCTTGGAACTCAAACG | |
| | AATGTAAAATCCTATGAAAATTAAAAAGCAGTACCACAAGTTACC | |
| | CCAAAAGTCCTTAGGTCAGTAACTGTTCCTGTTACAGGTAAGAGA | |
| | GAGCATGGATTAGAGGTGGGCGTGGGTATCCAGTGGACATGGTTT | |
| | TGAACCATGCTCCACTACTACTCACTATCTGAGAATTCTTAAATT | |
| | TATTAATCATTTCTATATTATAATTTTCTCAGTTATGAAATGGGA | |
| | AAACAATACCTAAATCACATGGTTGTTAAGTAAGCAATTGATTGT | |
| | TAAGCATTTGGTCATCAAAAATATTAATCCCCTTCCCTGATTCCC | |
| | TAGATAAATGATGAAAATACTAAATAAAAATAATAAAAATTTAAA | |
| | GTGAACATCTCAATTCTTATACTTTGTTAATTTCTACATGTATTA | |
| | CAAATCTACTAGAAATTACTTGGAATTGAGGAAATGATTACTGCT | |
| | TAATAATTCTTTGTGGTAGAGGGAGAGTTGGTATCATATTTATGA | |
| | GACAGCAGCCAATATAGTATATCTCAAAGGAAAAAATCCATTCTA | |
| | CATAATGCCAGAATTTAATAGTTAAGCATTTTATCTAGGTCACAG | |
| | CACAATAAGCAAGATGGATAATTAAAATAAAAGTATATTTCTCTT | |
| | GCATATATTTCTCATTTCATGTTTCCCTATCATATTTTATATCTT | |
| | ACCTTACTTCAAATACATATATACCTTCAATAAAACTGAGCCTTC | |
| | TTGCTTACCCAGGAAGTTTCATCATTCAGTAGAAATAAAAGATGA | |
| | CTTTAGAAATATTAAAATACAAAAATCTACACTGAGGTCTTTTGA | |
| | ATGCAGGAAAAAGAATTATATCACACACACACGTACACGCACGCA | |
| | TGCATACACACACACAGAACCTCTCGTTCTTTCTTAACATCTTAT | |
| | CAATCCATCAGTTTCACTCCCACTCCGTATCACCTGACTGTGCAC | |
| | AATATCTCATTGCCACCTCCCAGTCTTCTCCCTGCCTGGCACCCT | |
| | CCTGCTCTCCTGCTTCCACTTTAAACACCCTTCCTTCAGCTAGGT | |
| | CTTTTCTTTCAGGGATCCTCCCGTTGCTTTCTTATCTGGATCAAT | |
| | TTAGCCTTCCTCTTCTCCACCCATTAGTGGATAAGCACGACAAAG | |
| | ACACTAGAGTCAAATAATACAAACAGAATATACCTTAGATGAGTA | |
| | TGGTGATGAAAAGGATATGGATACTTAGAGTTTAGCACTATTCTC | |
| | TCAGCCACTCAGGAAAGCAACGCCTTTACAATCAATAGTGTTTCA | |
| | GGTACCAATCAATAATCTGTTATTGCTATTTTTAAAATCTATAAG | |
| | GTATCAGTAAAATGTAATTACTAGAGCAACAAAGATATCTTGTGA | |
| | AATCAAATTAGTATTCATCCAGCAACTGAGTACAAAGGTTTAAGG | |
| | GAGGATAACTACCAATACCAAAACATTTTAAGCATTTTGTTTTGC | |
| | CTCCTAAATATCAAATCATGTAAATGTGTGGTACATAAATTAGGA | |
| | ATTATATTTATGACATAGCTGCAGACATATTAAGAGAAATATGTG | |
| | CTTATATTTACAAGTATAGTACAGTTCTTTTTCATATTAGATACT | |
| | GTTGATGATAATCTGCATATAAAAATGCTCAATATTTTTTCACAT | |
| | TTATAAGCCATAAAATACAGCTAATAAAATGTGTTTCTACTTTCT | |
| | CATAAACATGGAATAGTGACAAACAAGGAGCTTTATATGAAAGCA | |
| | CCATTACAATTTAAACTCTCACAAGGTCATAATATATTGCACTAA | |
| | GCAGGAGAGTTCAGCTTATTTAAAAAAAAAAATAAACTCTAATGA | |
| | GGTTCTGGAATGCAGAGCCAAAGCATAAAGATGGAAATAAAAGAA | |
| | TTGCATGTCTTCTGAACTGACTTGGTTGATGATTTTTTTAAAAAA | |
| | GGTTTTGTGTCTTCTGACTTGGTTGATGATTTTTTAAAAAAACGT | |
| | TTTGTGGTAGAACAAATAAGGTAAATGAAATTCAGTATTTAGGAT | |
| | GAAAAGTTTTTCTAATTTCAGGAACAACATTGAAGAAATATTGAA | |
| | CTAAGCAGCTTTGAAAGAATCAGATTCCATTTGTTGAAATTTTTC | |
| | TGAGAATGAATTTTTTTAAGACAGTGTACACAGTTGCAGTGTGTA | |
| | TTGGTTATGGATTGTGGCAAGCTATATTACAACTTACCCAAGAAA | |
| | TAAGGAGGCTGGGCGTGGTGGCTCACACCTGTAATCCCAGCACTT | |
| | TGGGTGGCCGAGGCGGGCGGATCACGAGGTCAGGAGATCGAGACC | |
| | ATCCTGGCTAACACGGTGAAACCCCGTCTCTACTAAAAGTACAAA | |
| | AAATTAGCCGGGTGTGGTGGCGGGTGCCTGTAGTCCCAGCTACTC | |
| | GGGAGGCTGAGGCAGGAGAATGGCGTGAATCCGGGAGGGGGAGTT | |
| | TGCAGTGAGCCGAGATTGTACCACTGCACTCCAGCCTGGGCGACA | |
| | GAGCGAGACTCCGTCTCAAAAAAAAAAAAAAAAAAAAAAAAAGAA | |
| | AGAAAGAAAGAAGGAAAAAAGTCACTTGAAAAGAATACTGGACTT | |
| | TGTGTCCAGCTTGCATAGCTGAAAAGAATAAAAACCTGTCCACTT | |
| | AAACTCATTGCAAAAAGAAGATGTCACTCCTACAAATAGCAAAGA | |
| | GTCATGAAATTATTCTATCCAGAAAAGTATACATTTCATCCCTTT | |
| | GGATAAATTTTAGAAGTGAACTATGAATACATACGGTGAGGATAG | |
| | CCAGCTAAGAAGTCAAGAAGGATTTCTCAAATTTGCTGCTCAGAA | |
| | AGATCATACTCTCCACAAAACAAATAATAGCAGGCTTTCCAAGTC | |
| | AACCTTGAATCCAGCTTTCCTTTATCTTTCCTTCTTGTGAACTTT | |
| | CACTAGTTTACTATCTAACAATGAATTTGACGATAGCCACATACC | |
| | ATCTTATAGCAATATTTGTTATCATATCCCTTGTTATTTATCATT | |
| | CACCTGCTCTGCTTGAGCCAGCTACAAGTCACATGTCCCACGCAC | |
| | TTTTTCCTGTTTGATTTTTTACAGCACTTTGAGACATGTCTCATT | |
| | ATTCCTACTTGACAGGAAAGAAGCCATGGAAAGTTGAGTGACTTG | |
| | CTCCTGATCACAAATGCTGGCCAAGGAAGAGTCGAGTTTCAAATC | |
| | TAATGATCTTTCCACTGCACTCTAGATTCCTCATTTTGAACTATT | |
| | TTTTTATTTTTTGCACTATAGACTTTTTTCCACATTTTGAACTGT | |
| | TTTTTATTTTTTGCACTATAGACTTTTCTCTTATACCCAACTATA | |
| | TTGATGACTTCTTTTAGGCTAGAAACTTGTTTCACTTACTTTCCC | |
| | TTTCTTCAGATTGCTGCAATATTGGCCAACATGTATTGGGTACTT | |
| | ACTGAGTCAAGTACTGTGATTGTGCCAAGTATCTTATAGGAGGAT | |
| | TATCATCCTCATTTTTACAGGTGAGAAAGGAAAGGAGGTAAAGTC | |
| | ACACACAGCCAACAAAAATGGTAGCACCAGGATTTGAAACAAATC | |
| | AGTCTGACCCAAGTTGACTTTGTTAACCACTGTATGCACAGTCTT | |
| | CTTAGACATAGTAAGAGCTCTAATTGTGTTTGGTGATTTGATTAT | |
| | TATGACAAAGTAAGTAAGGGAAGCAGGGAGAATTATAAGAAATAA | |
| | GGCTCCACAACACTTGGCTATAGCAAAGCCCCTTAAAACTTCAAA | |
| | AGGTCACCCAAAGAATAAAGATCAGGCTGGGAGCAGTGGCTCACG | |
| | CCTGTAATCCCAGCACTTTGGGAGGCCGAGGTGGGTGGATCACCT | |
| | GAGTTCAGGAGTTCGAGACCAGCCTGGACAACATGGTGAAACCCT | |
| | GTCTCTACTAAAAATACAAAAATTAGCTGGATGTGGTGGTTGCCG | |
| | CCTGTAATCCCAGCTACTTGGGAGGCTGAGGCAGGGAGAATCGCT | |
| | TGAACCCAGGAGGTGGAGGTTGCAGTGAGCCGAGATCATGCCACT | |
| | GCACTCCAGCCTGGGCAACAAGAGCAAAAAACTCTGACTCAAAAA | |
| | AATAAATAAATCAATCAATAAAATAAAGATCAATTTGGAGAAATT | |
| | AATGCTTATTAATAAGCAATGTCTTGCACAGCACTTCAGTTTCTC | |
| | AATACATTACCTAACTCAATCCTTACAACAACACCCTATCCCCAT | |
| | TTTGTGGATAAATAAACTCATGTTCAGAAGGTTGAATAAATTATC | |
| | TAAGGTTAATAGTTCCTGACCTAGAGCTCAAATCTTCAGTTTCTA | |
| | TCATATTCTTGCCCTTACCCTGGGGTAGCTAACATTCACTCACTA | |
| | GTATTGGAGCTAAAATAAGGGAGAGAACATATAAATGAATACAAA | |
| | GGAGACATTCACCTGCCTTCTCTTTCTCCTTACATAGAGAAGGTT | |
| | GATTATCTGCTATTGTGAAGTTTGCCTTTTGAAGGATAGAAATGA | |
| | GAAGACTTTCTTAAATTTTGCCTCTACGCCAAGAAATTAGAGTGG | |
| | TACCACCAGTAGTTCCATTTTCAAACTATCACTGTAGCTAAAGCT | |
| | ATGTGGTAAGGGCCAAGGAAAAGAAGTATTCTTGCACTTCAAAAT | |
| | GCACTGAAATACCAGTCAGTAGCATAATATAAAGGAATTTAGTGG | |
| | AGAGAAGAGTTGACCTCAATCTGGCTCCAACATCTCGGCTCTTAA | |
| | CCCCTACCCTACACTTGTTCTTCATGGGGAAGCTAATTGGGCCAC | |
| | TGGAAGATTCAGCAGCTACCATTTGCAGCTGAGGGACAGCCCCTC | |
| | CCTGCTTAGCAACCAATGGATATGCATTTATGGAACACCTGCTAA | |
| | CTGCGACACACACTCCTATGTATGAGGGAAAATACAAAAAATGTT | |
| | AAAGGAGATGCCTTCCCTTGCCCTCAGGAAACTTAAGTATAGTTG | |
| | CAAAGAAATGATTAGCAGCAAACGAAACCATGGAGAAGTAAGGGC | |
| | TAAGGTCTGTGAAACAAGCCTAGAAAATAACCTTGTCCTTGAAAA | |
| | ACACAAAAAGAAAGAAAGAAAGAAAAGAAACTCCAAGGCCCTTGT | |
| | GAAGGAAACCATTAAGTTTGCTTCACTTCTGTGTTTAGGAAGACA | |
| | CAAACCCAGTCTTAATGAACCTCAAGGCCACAACTACTGGAGACA | |
| | TTTAGGAATTGTCACCACATTCTAATGTATATATCCTCTGTTTGG | |
| | CCCTTCCTATTAATATTTTGTAAAATTTTTGAAGATATGAGCAAT | |
| | GTTTAAAACCATGAATCCCCCTTTTTTTATAAGTAATATTTAGGC | |
| | TGAATAAACAAGAGAAAATAGGACATAAAGGGGAGCCAACGTGTG | |
| | CCTTCATTTATAATGTATTCCCAAGTTGTGAGTTTGGTTTATCAG | |
| | CAATTTATCATGCCAAATTCCAAGTCATATTTATCTATGCAGATC | |
| | AAACACTTGATTCTATTTTTGCCTTAATTTTTTTATTGGGTATGT | |
| | TTATGACCAAGTCATATGGTATTTTCTGTGACAGATAAAATGCAC | |
| | AGGTTATTCCAATCTGGCTCAGCCAGTCATAGCAACATGTAGTCC | |
| | TTCTCATGTCTTAAGAATGAGTATCAAGAATTCAAAGGGAGTTCC | |
| | AGATGGCATCCAAAAAGCTTACAGTTTATGCATCACTTATTCTAA | |
| | CAGTAGAAAAAGAATATTTGAAGCCAAAAATAGACCTTGCATGTA | |
| | GCATGTGGAAGAGTAGAAATTGCCCTGATAGTTAAACAATTTGAA | |
| | ATTCAAGACATTAATTTCTTTATGAAGCATTTGTCACATCATAGG | |
| | TAATATTTTATGCCTATCATATATATACTTATTATGAAATACAAA | |
| | GAAATTATTCATTCTATCTAAGACTTTGTATCCTTTACCAATATC | |
| | TCTCCATTCTCCCACCTCCACCCTAGCCCCTGGAAACCACCCTTC | |
| | TACTCTCTGCTTCTATGAGTTCTTTTTTAGTGAGATCATGCAGTA | |
| | TTTGTCTTTCTGTTCCTGTCTTATTTCACTTGACATAATGTCCTT | |
| | CAGGCTTATCCATGTTGTCACAAATGACAGAATTTCCTTCTTAAG | |
| | GCTGAATAGTATTCCATTGTGTGTATGTAGCACATTTTCTTTATT | |
| | AATTCATTTGTTGATGGATACTCATATTGATTCCATATCTTGGGT | |
| | CTTGTGAATAATGATGCAGTGAACATAGGAGTGCAGATATCTTTT | |
| | TGACATACTGATTCCACTTTGATGGGATATATACCCAGTAGTGGG | |
| | ACTGCTGGATCATCTAGTAGTTTTATTTTTTTTTATTTTTTATTT | |
| | TTTTTATTTTGAGACAGAGCCTTGCTATGTCGCCCAGGCTGGAGT | |
| | ACAGTGGTGCCATCTAGGCTCACTGCAATCTCTGCCTCCTGGGTT | |
| | CAAGCAATTTTCCTGCCTCAGCCTCCTGAGTAGCTGGGATTACAG | |
| | GCACGCACCACCATGCCCGGCTAATTTTTGTATGTTTAGTAGAGA | |
| | CGGGGTTTCACCATGTCTCGAACTCCTGTCTTCAAGTGATCCGTC | |
| | CACCTCAGACTCCCAAAGTGCTGCGATTACAGGTGTGAGCCACCA | |
| | CGCCTGGCCTAGTAGTTCTGTTTTTAATTTTTTGAGGAGCCTCCA | |
| | TACTGCTTTCCATAATGGCTCTAGGAATTTACATTCCACCAGCAG | |
| | TGCACAAGGATTGCTTTTCTCCACATTCTGGCTAACCAGTCTCCT | |
| | GTCTTTTTGAGAACAGACATTTCAACACGTGTGAGATAATATCTC | |
| | ATTGTGGTTTTGATTTGCATTTCCCTGATGATTAGTGATCTTGTG | |
| | CCTTTTTTCATATAACTGCTGGACATTAATATGCCTTCCTTTGAG | |
| | AACTGTGTATACAGGAGAAAATAATCACTTCTCAGAGGAGCTTTC | |
| | ATTTCAAAATATCCGGGAAAAAAATAGAAAAAATGGAAAATTTAT | |
| | CCTAGAGTAAGTTGTCTTTTATATTTTGACCCTGTTTGTGACATA | |
| | AACTGGATGATACAAAACTGGAATGCAAAGGCTTTAGGAGGATTA | |
| | CTTACTTACTTGTATATTGCTTTAGGTTGTTTGCAGAAAATTATA | |
| | CTAATTGAAGTTCAGGCTATGATGTGATAAAATCTATGTCAGGAG | |
| | ATGAGTCTACATGCAAAGTTTGAGGAAGTGACATTTGAGTTTCAA | |
| | AACAAAAAAGCAATTTTCAATGTCATATCTAGGTTAACCCAAAAG | |
| | ATTTCTTTCACCCTATTTAGCTGCCTCTAAGATGGATGCTGAGGA | |
| | TAATTACACTGTAGAACAATAGGACGATGCTTCACACTCACCTCA | |
| | CAGGCTCTGTTATTCCCACATACTGCCAGAGATACTCCAAAATAA | |
| | AATCACTGCAACATCAGGCAGTTATAAACCTCAACGGTATTATTT | |
| | TCTATTTATATACAGTATATTTTATATTTTACAAGTATAAAATAG | |
| | AATATATTTATTCTATTCTCTTTGACACAAAGTGACCATAAGACA | |
| | TATTACTTAAGTATGACTAGCAAAGTCATGGGGCTTGTCATTCAG | |
| | GAGGAAACTCTTAACTAACTGTTCAGTTTTTGTTCACTGCACCAT | |
| | TTACATAAGCCAAACTAATGCTTCACACTGTGCAAAACAATGCAC | |
| | AGTGTTGTGAATGAATGGCTAAAATAAAACTCTAATGAGTGGGGT | |
| | TTGAAAAATGCAACTTTAGAAAACTGTTGAGAAAATGTTGCACAC | |
| | TGCGCATTTTACAAAATTTCGTTGAAGGACACTGGATATTCTTTT | |
| | TAGGATTATGGAGGGAAGCAAAATTTTGGCTCCTACATGCAGTTT | |
| | TTGTGGCCTTTGCCTGAAATAGTCATCTCCCATTAATTATTTAGA | |
| | TATCATTCATTTCCTAAGACAACATTTAGGGAGACTGCCTTAAGT | |
| | ACAATTTGTACACTACCCAGATAAGAATTCTTTTTGGTGAAACAT | |
| | CGATAAATATTACTTGGCAGTAACACCAAGTTAAAATATTTGTTT | |
| | CACAGTCGACGTTAATAACTATTATAGATAAAGTGAATTTTATAA | |
| | GACATACTCAGATCTAAAACAGCAATATGGAGCTCTTCAAATCCA | |
| | TTGAAACTTCATACCAGCCTACGGAAGTAGAGGTTTTTATGCAAA | |
| | CTCTTCAAGAAATATGCTCTGAACTTTTAATTCCTTAGATTGATA | |
| | GAGGAATTAAATCATGATATAACTAATAGGTTTGTGGTACAAATT | |
| | GCTGCTGCTTAATCTGACTCTGTGTCTTCCCAGTGTTCTATATGA | |
| | ATTAGATATTCCATTATCTAAAGACAATCAACCCCATCCCACGGT | |
| | GATAGCTCTAGGACTCCCTTTGAGTTCATTAAATCTGTATTCTCA | |
| | GTCTCCAAACTTCTGGTTAATTCAAACAGAAAAGTCAACTGGCCC | |
| | ATGAACTAAAATAAAGTCATCTGAATTTTTTTTTTATTTTGCAGT | |
| | GTGATAAAAGTCTCGCACTTTTTATTTCTGAAAGTTTCTGCTTTC | |
| | ACTGAGAGCATAATAGGCTATCCACCCTTATGCAATCTTACATAC | |
| | AAAGTCATAGTCAGGCTAAATTCAAAAACACATGTGAGATAGAAG | |
| | TCAACGTTTATTTTCTGGAGAAAAGCCACACATTACAACAAAGTG | |
| | AACAATGAAGCTGGCATCCTTATCACTGGTGACCAAAACATTTGT | |
| | GACTCTGGACATTGGCCCCACAAATGCGATAAACATTCTGCATAG | |
| | GAAGTGAGTTTTGCTAATTAAAAATGGATCCAAAATACTTTCTAC | |
| | TCTTCAGCCAAGAATTAAAAAGTAATAGGGAGGAATTGAAATCAC | |
| | TTGGGTGCTACATTGAGCCATTCTGGAGAAGCAATTCAGAGAATG | |
| | TCATGGCAGCCTCAAATTGCTGCTCAGGAGCATCCCAGCTTAGAA | |
| | GATTGCAGGAAAGGAAGAGCAAAGTCATTCTTACATGAGAACTGT | |
| | CCTTAACCAGATGAATAGACTCTCCATTTTTTACCCTGGCTTTGT | |
| | CTCATTTAAGTCCCAACCAATCTAGCTATCATTTTAGGTTTTACT | |
| | ACCTGCTAGTATTTAGGAGCTTAGGGGGATAAAAAAATCCCTCAA | |
| | TACTCAGAATTAGACTTGGTGATAAAAATCTTGACACATAAACAG | |
| | AATAAAGCGCTTTCATTACTCCTCTAAACCACAGTGTCATTTGGT | |
| | CTCTATCAAGGACTGTAAGAATTTCTTTCATCAGGGGAAAGAAAA | |
| | AAAGGACAAGAGCCTGCAAGATGTAGCGGAACTCTCATTAAACAC | |
| | AGCAGGAGCTTTAACTGGAATCCAGAGTAAGGTGAGGTACCAGGT | |
| | TACAACAATTTACTGCTTTTATTACAATTTTGATCACAAGGACTG | |
| | ATTCATGTCATCTAGTTTCTTTTCCTTGTCACTATCACTGGTGCT | |
| | AAGAATACATCAAATTGAAATTTAAGAGCCTCATATGTTTCTGTA | |
| | TAACCCAGTGATGGGTTGTACTGCTTTGACCTTCTTAAATGTCCC | |
| | TTTATTTCATTTGATATCCATTCCCATAGAAAAACTATAATGCTT | |
| | TGGTTGGTCAAAATATTAATCTTTCAAAACCTCCCTGGCTTAGAA | |
| | AACCAAATTTTTGTAGAGAGAGATGGGTAGAATCTAATTTTATTC | |
| | TAAAGCAATTAGCATTACATCATCACAGCAGAAATATCTAGAATA | |
| | TTACCTCATGTCAGTGATCTTCTGATATGTTAAAAAGGGTATTTT | |
| | AAAATCTGAGTTATTTCTTTTTCTTTTTAAAGTTACATCATTAAT | |
| | TACATACTCATCAACCAAAATATTTTATGCTCCAAATTTGAACCG | |
| | ATATAGTATGTAAGAAGTGTTCAAAATGAAATTATTTTGGTCTAT | |
| | TTTGTCTTTGAAGAAGATCACAGGGATGGACCTCCCAAAAGGATT | |
| | TTTAAATGGGATTACATATCTGACTTTTAAAAAAAATTATCTGAC | |
| | CTTGAGTTATAGTGCCCCAAAGTAAGCAAAGTTCCAAACACACAG | |
| | TATCATCAGAATTGAGTTAAAATTATCACCAGGGGCTTAATTTCT | |
| | GAAATTAAAAAGGAAATGTTATTTCCTTATGAAAAGAAAAGGAAC | |
| | CAAAAATGAACTTCAAGGTAGCTGATTTCTGTCTATGTTAAGACT | |
| | TAGGTAATGGGAGAAAGGGAAAAGGAAGGACAGAATTAGGAGAGG | |
| | AGCAGTGTTTAACAATTGCGGGTGCAAGACTCAAGTTTTTTAGAA | |
| | TCCATTAGCAGAGAACCCTATTTCTCCCATTAACTGCTGTCCTTT | |
| | TAAATCCTGGCACCAGCTCTGAGGACTGCAGGGTCCATAGCTAGT | |
| | GCCCCACTCTACCCAGTTTAAAGACACCACTGCCTGGAAATGACA | |
| | GGGGTTTTTTTCTTAAGGAAAGAGGTGCTTTCTGCCACGTATATA | |
| | TAAATTGGTAAGCTTCAAATAAAGTGCTTTTGTCCTTTCTGTCTA | |
| | TCAGAAACTGTGCAAATCGAATTGCTGTAAAACCAAGGGCAAGAG | |
| | ACATCAATCCTGCATTCTATAGCATCTGATTTTATCCTTTATCCC | |
| | CAGGCACATTTCAAAAGGAAAAAAATGAGGTTGCATTTAAATTGA | |
| | GTATTTGGGACTTGCCAGGAAAACCTCCCGCTAGACTAATATGAT | |
| | TGCAGGGAAAACAAGAGAAAGGAAAAGTGGAGAGGGAGTGTGCTA | |
| | ACAGATCCTGGGCCTCGTCAGCAGAGCCGTCCTGAGCACAAGGCC | |
| | ATGGTCAGACATCTGGTCCCGCGAATGACGTTTTCTTTATGGTCA | |
| | TTAAGAACACCAGTGTGTCGGGACACAAACAAGTATTCCTTTCAG | |
| | GGATTATGACACATTTTCTCCCAAAGTAGTATATTAATGACATTT | |
| | CCAGAGCATTCTTTACTATCTTTTATATGTGATCAGGAAGACTAA | |
| | TACATATCACTACTTCTTTTACACACAGCATTAGCCAAAACTAAA | |
| | GTGTCAAATACAATTTTGCCTAGGATGAATAAACAGAAGAAATTT | |
| | TTATGATACTGCACTATCAATTCCAAATTAAATAACAACAAAATG | |
| | ATAAGTGTTAAAATTCATATTAATGATTGTTCCCACACAAGCCGG | |
| | AAAAAATCTTTCTAAGAAGTCTTTCATGAGTTAATCCCATCTTTC | |
| | AAAGTGTTCAGTGGCTCCGAATTCAGTTACTGTTTCCTATCAGTT | |
| | CTTCTTTCATTAAGTCTCTTCCCTTTTTTTTCTCTTTGCACTATT | |
| | TCCCTTAGCCGGGTACATAATCTGCTGTGCTTTATTCATTTGTGT | |
| | CTTAAGTTTGTTTCCCGATGACATACCTTTCCAGCAACGCCATCT | |
| | GGGGAGTTTGGGCAACTGTACCACGTTAGGAGGAAACCCTTCTTC | |
| | ACAGGAGAGTGTGCCTTTGCTGCAGGGAAGGAATTAGGATTTGCT | |
| | TGGACTGTGGTTGCAGCTGGCTTTTAAGGATCTCCTTAGAATGCA | |
| | AGCAACTCATCAATGAGAATCTCTGCAATGGTTGTCACTGGGTAG | |
| | AGTCATGCTATGTGGGGTCATAGCCTTTGAAACAAATAACAGTAA | |
| | AGATAAAAATGCTATTAAAGGAATCACCACCCACAGAGGTTAACT | |
| | GGGTTTTGTCCCCAGACCACCTCGAACAAGAAAGAACATTTTTAT | |
| | CAGTCATTTTCTTAGTTTTAGCTGATAAAACAAAGTACCATAGAC | |
| | TAGGTGGCTTATAAACAACAGAAATTTATTTTTCACAGCTTTGGA | |
| | AACTGGAAGTCTGAGATCAGGCCGCCAGAATGATCAGATTCTAGT | |
| | TAGGGCCTACTTTGCTTTTGCAGACTGCCAACTTCTAGCTGCATT | |
| | TTCATGTGGCAAAAGGAGATTGAGCTAGCTCTCTGGTCTCTTCTT | |
| | ATAAGGACACTAATCCCATTCATGAAGGCTTCACCTTCATCATCT | |
| | AATTACTCTCCAAAGACCCCACCTCCAAATACTATCACATTGGGA | |
| | ATTAGATTTCAAATACAAATTTTGCGGGGACACAAATATTCAGTC | |
| | CATAATAGTAATGATTACTCATTATACATAGGGCTCTAAATGTGC | |
| | TAGCTTCTGATAGTTTTTACACTCACTTCTCTTTATTAGCTTGTC | |
| | AAGCATAATTAGGGCAGTGGCCTTACTGAAAATTATTGAATTTAG | |
| | TTTCCTAAGGACAGATATTGAGGAGTTTTTTCTTCACTAAAAATT | |
| | CACGTTCCGATACAGCTTTCATCTGTTACTACTTTGTGAGATGGA | |
| | AAATCTTTTATTTTATTTTTATGTTTGGATTGACCCTTCTTAATA | |
| | AAGTCGGCATGTAATATGCTTCATGTGTTTCTAATATGTGCTTAA | |
| | TTTTGCAAAATGTTTTGCATACCAGAATGCATTTCTCTTCCAAAA | |
| | AAGGTACCAGCCTACAAAACCTTGCTGTTACTGTTTTCAATTAGT | |
| | TCATGGAATTAAATGTATTAAATGTTTTATGCTCTGGCAGAAATT | |
| | ATGATTCTCACTTAACTCCATATAAATCTGGATCTGCCTGGGCCT | |
| | TTATAAGTGACACAATTTCATTAACTGAATAAACAAATGATACAA | |
| | AGAAATTTGGTTTAGCCTTCTAAAATTCCAAAGGCGTTCAACAAA | |
| | ATATCTCAGAATGGATGTTCCAGGACTTTTATGGCACAGGACAAC | |
| | ATGTATTGCTTATTTTAAGAAAATAAGCTAAATAGTGAGGGGATT | |
| | CTTTTAGCAGATCCTCAGGATGTGTTAGGTTGAATCATAGGCAAA | |
| | TGATATTTGATCATTGCACCTGTTAACACATTGAACCTCATCCTA | |
| | AAATTGTAGAGCTAGAAGAAAGCCTTCTGGCAGTTTTTAAATAGA | |
| | TTGATTTACTGCAATTTATCCAGAAGCTTCACCGTTGTCACTGGC | |
| | TACATGTGACTTTGGCCTCTGTGGGGCTATATCCTCATTTGTAAA | |
| | ATTGGTGGTGAGGTAGGTGGACAGTTGACTAAATAATCTCTTAGA | |
| | ATAATTCTAGTATCTGTGGATCTAAAGCATCCAGGGGTTGAATAT | |
| | GTTTCTTTCTGGCCAAGAAAAGATGCACCTGTCAATAATGCCCAA | |
| | ACTCATCTTCTGAGAATCCTCTTTCCCAAGATACCCACTCTCCCT | |
| | TGGGTTATATTATAGTAATGATCAGAAGCCCCTGCCAAGAAGAAA | |
| | CTGTTAACCTGGGAGGTCTATATTTTATTTCACAGCCATCTGTTT | |
| | ATACTTTCTCACAAGTTAGTGCACAGTATACCCATCATTTTCTAC | |
| | CATTTTCCTTAATTTATTAATTTTACTAATTGCATAATTAACAAA | |
| | AGTAAGAAGATTTTACCTCCTTATCCCCATCTGGTAGTTTGCAGA | |
| | TACTTGGCCTGATGACAACTGACAGTGATGAGATACTCACCAAGT | |
| | TTACCAGGGCAGGAGGCTTCCTAGAGAAAAAATGAGAAAATGAAA | |
| | TGGGGAAGGGGAGTGAAGGATTGAGGAGGTGACAATCTGGACTCT | |
| | TGCAACTGCATGGCAAGGTTGGCACACAAGCTGGGTTGCAACGGA | |
| | GGGAAGGAGATCCTTATCAGATGTAATCAGAGCTCAGATCGAGGG | |
| | CTTTGGTGTGTGTAGAAAGAGGGAGAGACAAAGAACTTAAAACAG | |
| | AGCTGCCATTTGACCTTGCAATCCCATTACTTGGTGTATACCCAA | |
| | AGGAGAATAAATCATTCTATTAAAAAGACACATGTGCTTGTATGT | |
| | TCATGGCAGCACTATTCACAATAGCTAAGACATGGAATCAAACTA | |
| | GGTGTCCATCTATGGCAGATTGGATAAAGAAAATGGGGTAAATAT | |
| | AAAGCATGCAATACAACATGGCCATAAGAAAAAATGAAATCATGT | |
| | CCTTTGCTGCAACATGGATGCAGTTGGGACCCATAATCCTAAGTG | |
| | AATTAACACAGGAACAGAAAACCAAATACAGCATGTTCTCACTTA | |
| | TAAGTGGGAGCTAAACACTGAGCACACATGGACATAAATATGAGA | |
| | ACAATAAACACTGTGGACTACTAGAGGGGGGAAGGAGAGAGGTTT | |
| | GTAAAACTACCTATCAGGTGCTATGCTCAATACCTGGGTGATGGG | |
| | ATTTACACCCCAAACATCAGCATCATTTAATATTCCCATGTAAAA | |
| | AGACTGCACATATACCCCTTGTATCTAAAATAAAACTTGAAATTA | |
| | AAAAAAAAAGAAAGAAAGAAAGAGGCTGGAAATAGAGGCTCACAC | |
| | CTGTAATCCCAGCACTTTGGGTGGCCAAGGTGGGTGGATTGCTTG | |
| | AGCCCGGGAATTCAAGACCAGCCTGAGAAACCTGGTGAAACTCTG | |
| | TCTGTACAAAAAATACAAAAATTATCCAGGCATGGTGGAGCGCAC | |
| | CTGTAGTCCCAGCTAATGGGGAGGCTGAGGGGGGAACATCACTTG | |
| | AGCCCAGGAGGTGGAGGTTGCAGTGAGCTGGGATCACACCACTGC | |
| | ACTACAGCCTGGGTAACAGAGCAACTCTGTCTCAAAGAGAGAGAG | |
| | GAAAGAAAAAAGAAAAGATGGACAGATAAGAAAATGCACTTGGAG | |
| | ATTAAGAGAAAGCAGCAACATAGGACCCTGGATAATGTGTTTGCT | |
| | TAATAACTATCCTGATGAGTTATCTGACTATTCCCAAATGAGTAC | |
| | GTGGCAATTCAGGCTGAACCATCAGAGTAGCCCTCCGGAATCTTA | |
| | CTTATGTACAATAGACCTGCATGCACATTTACTAGAATGAGCCTC | |
| | TCTCTCTGGTAATCATGTCTGCTTCCACTAATTCCATCTGTTTCC | |
| | TCTCTCTCCCTCCTATCCTGCTAGATCTTAATTCCTTCGACCTTC | |
| | CTTTGTTTTTCTAACTCCCTTTCTTTCTCTTGTTATTTAACCTGC | |
| | TATACTATGCAATTGATCTCCTCTGCACTAAGGAACATGCACTTC | |
| | AGAATTCTGTTGACATCTTGCATTCCTTTATATTTAGTGAAAGAA | |
| | TGCAAAGGAGTCTACCTGGCAATATTCACTCTGCAGGAGGCAATA | |
| | ATTATTATTCAAATTAAAGGAAGCAGTAAAGAGAAATTCAGAAAA | |
| | AATGAAATATACTAATCTTCAGCTTTTCATTTCAGCCTACAAGGA | |
| | AAAAATGAAGGAGCTGTCCATGCTGTCACTGATCTGCTCTTGCTT | |
| | TTACCCGGAACCTCGCAACATCAACATCTATACTTACGATGGTGA | |
| | GTAACCTAGGATAGACATACCCCTGCTAGCTAGATCATTTGGAAA | |
| | GGTTGACATATATTTGTTTCTTACAGCTCCTGATATAATTACATC | |
| | AATATTTTGTAGCTCTCACTATTGACTTGCCGTGTCTAGCTATTA | |
| | TGTCCAATTGATTACCTATTGCTGAAAACAGTTTGAATTTGGTGC | |
| | TAATAACAACACATCAATGTCTGTTAAGAAATGTGGATGGATTCT | |
| | TATTAACAGCCACATCCAGCATATCAACATCCACAATATGTCTAA | |
| | GGTCTTTCTTTGCAAATAATTTAATAGGCTAAGCCATAATTGGAG | |
| | TAGATCATAATTTGTAAGAAAATGCTTTATACTTAGAAAACTCAA | |
| | GAGAAAGAATCAACAACCATAATTGTTTTTGCTTTATTGTAGTCT | |
| | TTATAAAGTTTCTATACTTTGTATATACATGTCAACCAGCTAATG | |
| | ATAATAATAATTGGCTCAATAAATAAAACTGACTTACGACTGAGG | |
| | CCCTAGATAAAGAGGGTCTGAAAAGAAAAGCCTAAAGAATTAGCA | |
| | TGGCAATTAACATGATTGAGGTGCAACTCTTTAGGTTTGATTTAT | |
| | CCTGATTCATTTTGCTTACTTTGGCTCTGCCACAATCCACATGAT | |
| | CTTGGTCAAATAGATACTTGGATTCTCTAAGTCTCATTTAACTCT | |
| | AGCATCTTCCTCTTGGAGTTGTTGTGAGGTTTAAACGGTTTAATG | |
| | TAAGTCAAATATGCAAAACCAAGCCTAGCTCATTATATCACTCTA | |
| | CAATGATAGCTATCATTATCAACATCATCCTTACCTAATTCAGTC | |
| | AATTTAACTAAAATATTTTATACAGTTCTATGTATCCTAGATATC | |
| | CCTAAGGCATATTTTACTAACTCTCAGGCTCACAAATATTTTTCT | |
| | TTTCCATATATGTAAAGAAAGACATTAATGACAAAACAAACTGAC | |
| | CTTGTGGCAGTTAACCCCTTCTGCACCTTTAAAGCCTATTCAAGG | |
| | ACTCAAAGGCATTTACCTTCCAAAGTTATTCTATCGTAGCACAAA | |
| | AATCATAAATGCTAATTAACTGTTCCATAAGGAAATGTCCTCCAT | |
| | GTGAAAGGAATTCTGTCTCCAAACAAAACATTCATTAGAATGCAG | |
| | GGCCAATGCCTACTTTGTACAAATTCATTCGGTCAGCAAATAAAT | |
| | TAGACAGACCTTTATTATTTGCTAGATGTAGCTGTGAAGAAGGAT | |
| | CCAGCTATGTTTCTTATGAGACTAATGTCGAACTATGGGTTGTCA | |
| | CTGAGGATCCAGAGTTCCATAGGGCGTAGTCCTCACCTTCAAAGA | |
| | ATTCAGGGCTTAGTAGAAGAGTCTTACACAAATGACTAGAATGTA | |
| | GAACACAGAGTGGTTAGGACAAAGGAGCCAGGGATGGTTTTTGCT | |
| | GGGTTAGGGAATGAAAAAAGGGGAAGAAAATATGTGAAGTTATGT | |
| | GTGAGCTGATTCTTGAAATAAGCTGTTTTTATTTGCCTGCGTTCT | |
| | CTTATAATCCTTTTCCATAGGCTTCCATAATTTTTATTGAGCTGT | |
| | ATTTAAAGTTGAATAGATAATTCAACATTTCTCGTAAACTGTGCT | |
| | TCCTAAAAGAGTCCGTAGAGAATTTCAAATTTCTGCAGTCTTTAA | |
| | CTTGACCTGGTATTTCTATGTTAGATAATAACGTGACTTGTTTAT | |
| | TGCAGGCAAACATTATAACAATAAATTATTATTATTGTTTACATT | |
| | TGTAAGCACTAAGTATATGGCTTGTGCTTTGCATTCAGCATCCTT | |
| | TATCATTTAATCTTCACAACCACCTTAGAAGGAAGGTACTCTTTT | |
| | TATTTCCATCTTTTAAATGAGGAAATAAAAGCATAAAGAAGTTAA | |
| | TTAACTTACCTAGTGTCACACAGCTATTAAGAGGGGCTTACTATT | |
| | TGGATGCAAATATAGGCAGTTCTAATTCCAGAGCCTCTAATCTAA | |
| | GGCATTTAAAACCCCATCACCTTATCAAATAAGCTGTTTTTATTT | |
| | GCCCGTGTTCTCTTATAATCCTTATCCATAGGTTTCCATAATTTT | |
| | TATAAAATTGTATTTAAAATTTAAGTATAATCTTGGATGCCATCA | |
| | GGAAAATGAAAAACATTTTTACATTTGTGAAGGAAAAAGCCCACA | |
| | TCATTTCCAATATAGTTATTGAGTTAGTATTATCTAGACTATCTA | |
| | TTAGCAGCTAAGGATCTGAGGTCAAGGCCTGCCAGCCTGGCATTT | |
| | TACTTGACCACAACCTCCATGTGCACTAACCAGGCTGCTAAAAGA | |
| | ACATTAACGGGAACATAACCTGCTGGCTTGGTTGCCACAATTTTA | |
| | AAAAGACGTTAATAAATTAGAGAGCACTTAGAGGTTAGGAAATAA | |
| | TATGGTGGTAAAGATCTAGAAACAGTGTCATTCTGGGGCACTTGA | |
| | AGATGTTTAGCCTGGGGGAACAACTTGAAATGGAACATAACTGTT | |
| | TTCAAATACTTGAAAAATGGTGGTGCACCACAGAGAATGGCCTAA | |
| | TCATGGGTAGCTTCAGACTTCAAACAAGGATCAGTGGGCTAAAAC | |
| | CAGAGAGATGGAGTTTGGGACTCAAAGAATGCTCATCTGAAATTG | |
| | AGGGCTGACCAGCGAGGTTCTTTTAAAAATCATTGCATTTTACTA | |
| | AATTGTGAGTTCTGTAATTATAAATGTCCTAGCAGGTGCTAGCTG | |
| | TCATCTTTTCTATTATAAATTATACTATTTTATGTTATAATTTGT | |
| | ATTATACAGGCTTAAAACATAAGGGTCTGATAATCTGCTTATCTT | |
| | TAATACATAAGCCACTGATAGAAAATAAGTGGCTAACCATTCTTC | |
| | AGTTCTTTTTTTAATTGACAAAAATTGTATATGTTTGCGGTGTAT | |
| | GGCATATTTTGAAATATGTATACATTAGAGAATGGCTAAGTGAAG | |
| | CAAATTCACATATGCATTACCTCACACACCTGTCATTTATTTGTG | |
| | ATGAGAACAAAAAATCTACTCTTTCAGTGATTTTCAAGAATACAG | |
| | TACATTGTTATTAACAATAGTCAGCATGGTGTACAATAAGTCTTC | |
| | TGCGGCCGGGCGTGGTGGCTCACGCCTATAATCCCAGCACTTTGG | |
| | GAGGCCAAGGCTGGCAGATCACGAGGTCAGGAGTTCGAGACCAGC | |
| | CTGACCAACATGCTGAAACCTTGCCTCTACTAAAAATAGAAAAAT | |
| | TAGCTGAGTGTGGTGGTAAGCGCCTGTAGTCCCAGCTACTCAGGA | |
| | GGCTGAGGCAGGAGAATTGCTTGAACCTGGGAGGCGGAGGTTGCA | |
| | GTGAGTCGAGATAGTGCCACTGCACTCCAGCCTGGCAAAAGAGGG | |
| | AAACTCCGTCTCAATAATAAGTCTCTTGCATTTGTTCTTCCTGTT | |
| | TAACTGAAATTATGTATTCTTTGATCAACATCTCCCCAGTCTCCA | |
| | CCCCTAACCCCTGGTAACCACAATTCTACTCTGCTTCCGTGAGTT | |
| | CAACTTTATGAATAGTCCACATGTAAGTGAGATCATGTGGTATTT | |
| | GTCTTTCTGTGCCTAGCTTATTTCACTTAGCATAGTGTCCTCCAG | |
| | GTTCACCCATGTTGTCAAAAATGACAGGATTTCCCCCAACTTTTT | |
| | TAAGGCTGAACAGTATTCCATGTGTATGTGTATAAATTAGATTAG | |
| | TAGATGTTGCCACTCCCTCCTCCACCACAGTGGCTCTATCCCTGG | |
| | CTCCTGGCTCCAGCCGAGTACACTAGAGGAGGATATTCTAAACAG | |
| | CAACAACACAGGAGCAAAGACATTACAATGGGGTGTTGTCTTATT | |
| | GCCCCCATTAGACTGTAAGCATCTTGAAGACAAGGACCCCCATCA | |
| | CAGAGTGATGTTGTCATCCCTGGAGTGGGCACTGTGCATGATTGA | |
| | TGACTGGAAGCAATGAACATACAGAAGGGCAAAACAGAAATCAGC | |
| | AGGATGCTTTGCATTTCAGCATTGACTTTGCCAAATATGCCCAAC | |
| | TGTTCAGGGAGTTACATTGGTTCTAACGAAGCTCCTGTGATTCCT | |
| | AAGCACAGGAATGGTGATAATATATATAATGGTGCATGCATATAT | |
| | ACGCATATCTAGATAATGATATCTCATTATATGTGAGAACTGAAG | |
| | AACTCCGTTATGTTTCTCGTCTAACCAAAAAGGGCCTACAGCTAC | |
| | GATAATTTCCAAACAAATAAATCTGTGCTACTTGATTTTCATGCA | |
| | AAGCTCATATTTGTTCAAAAGGAAAATAAAGCTTAATTTAAAATC | |
| | AATTTAGGCTATTTTTATCTAAGTATGCTTACCGTTATTCAACTC | |
| | CCTGCAGATATTGTCAAATTTCTCAATATGGTAAATATTTATTCT | |
| | GTTAAAATATATCCATAGTTACACTAAAGACAGAGAGGTCTTATA | |
| | TGTTCTAAACAACATAGAGCAAATGCTCATAAACAGCATTTTATT | |
| | CCTATCTCCCGGAATAACAACGCTACTTCCAATTGCTGGAATCTA | |
| | AATTATTAAAATAAACCCATGCTGCAAGCTTTGTATGCTTAACAT | |
| | TCTCAAATGTTCACTTTTCAGATATGGAAGTGAAGCAAATCAACA | |
| | AACGTGCCTCTGGCCAGGCTTTTGAGCTGATCTTGAAGCCACCAT | |
| | CTCCTATCTCAGAAGCCCCACGAACTTTAGCTTCTCCAAAGAAGA | |
| | AAGACCTGTCCCTGGAGGAGATCCAGAAGAAACTGGAGGCTGCAG | |
| | AGGAAAGAAGAAAGGTAACTTTTTCCATAGGTTTTCCTTCTCTCT | |
| | CTCCCTCCCCTGCTCCTCCCTCTCACACACTCGGGCACACATGCA | |
| | CGCACACACACACACACACACACACACACACACACACACACACAC | |
| | ATACAGAGAGCAATGACAGCTGAACCTGTGCCATGCCAACATGTA | |
| | TAGGTTTTCAGTAGACACAGAGCCAGGCTAGTTGGGGTAAAAACT | |
| | GTAAGATAGATGCTAATTTTAGGCTAGCCAAACCAGAGCTCTCAG | |
| | AAATCCAAAGAGCTTCAGTGCTCTAGTGCCCCTTCCCGTATATTG | |
| | AATCCCCTTATTATAAAAGCCTCCCTTCCCTAGACCATCAGGCAG | |
| | AAGCACTGTAGAGAAAACACAGCCCTGGCGAACTCCAGTGGTGGG | |
| | GAGGGGAAGAAGTGCTGCTTCCTCCCTCTCAGGATCTGTGTCACC | |
| | CCCTTTGTCAGGCGTGGTTTTCCTTGGAATTACAAATTACCAGAT | |
| | CTTCCCTCCAAGATCTTTCCTGCCCAGGGTAAGGGCCAAGAGCTT | |
| | GCCCCTTTCCTCTTCAGAGTCCCACTGCCTGCCCTGGAAGTTGGT | |
| | CCTTCCAAGATCAGGACCTTCTCTGAGTTCTTTGAATATGTTCTT | |
| | TATCTTTTTCTAAGACTTGATGGGGATTTTTCTCTTTTTGCCATT | |
| | GGTCCCTGCTTATATTAAAGAGCTTTCCTTTTGCCAAATCTTTAC | |
| | TTTTCCATAATCACATGGCTAAGAAGAGCCAAGGGTATTATTTGA | |
| | GAACACTTAGAAATCCTAGGGACTGTGTACACAAACAGAAGTTGT | |
| | TTGAATGTGTCTGTTCCAACCATGTGGTTATGGTAGTTAATCCCA | |
| | TCAAGGTACTCACGATCATCCAAAAATGGAATTCTTTTATGTAAT | |
| | TCATCCCCACATTGTATTTCCCAATATTTTTTATGATATAATTTT | |
| | AGAATCAGGTAATCACTAAGAACATGTTCCCTGCACAGTTTTATG | |
| | ATGTTTTCTCTAAAAAGTCAGCCAAAACTTTGGACACTTCTATGT | |
| | TGGATAATTAAAAACAGAATGAAGATAATCCTCCTCCTAAAGATT | |
| | GAATTCTCCAAGAGAGAATGCAGGACAAACACAGATGTGCTGTGT | |
| | ATAGTATATGTGCATATATACATGCATATATGTACACAAATATGT | |
| | GTATTATCAAATAATGAGGCTCAAACATTAGAAATCCTTAGATTA | |
| | AATTTTCTAAACAAGAAAACACTAATCTTTGTAGTTGAAAAAAAA | |
| | TCCTCCTATGATATGTAATATGCTGATCTCAATTTTCACCTAAGA | |
| | GTGATGTTCTCCAAATGTCCGATGAGCATGTCATATATATATATA | |
| | TGAATTTTTATATATATAATTACAATGGTAATTGGTATATAGAGA | |
| | TATCTATATTATAGATATATATAGCTATCTCTATATATTACATAT | |
| | ACCAATTATAGATATAAATATAACAATGGTAACTGGTGTATATGT | |
| | GATGTGTATATATGTATATGTATACCATAATTATATATTAATATT | |
| | GTATATATGCCATAATTATATATTAATATTGGTATATATACACCA | |
| | TGATTATATATTAATATTGGTGTGTGTATGTGTGTGTGTATATAT | |
| | ATATATATATATAAAATACTAGTTATCATTGTTCTAGATTTAAAA | |
| | AACAGGAACCTGAGCTACTAACTCGACTATATATATATATATATA | |
| | TACAGGAAGTTGCTTTAAAACATTTTTATCAGCTTTTTTATTGTT | |
| | ATTTTTAGCTTTATTCTCATAGTAAAGCTAAAATAAATTATTCAA | |
| | CATTATCAAAACTTTGCTGCCAGCAGATGTAAGCAATACCTAAAA | |
| | CAGTGGAGAGCATGTTGCACCCAAAGCAGTTTAAGCTCTGACCCA | |
| | AGCACTGGCATCTTATAGGCACTGGGTAGAGATAAGAGTCATAGG | |
| | TCGACATATATTGAGATGCTATGACTTGATTAGAATATGGAGTCA | |
| | GTGACTGAGGTGAAATTAAAACTCAAACCACAATTCAACATCCTG | |
| | ATTTAGGATGTTGCTGGTGTTTCTAGGTACTACACTTAATTTGAA | |
| | AGAAATTATTGAGGATAAAAAAAGAACTGGGATCAACAAAATTAA | |
| | CTAGGTGTTCTTATAAGAGTCCCTGAGGTTACTAATTAATGAAAC | |
| | TGATAAAGCTCCTGCACCCTGACAGCAAGAAATTATCAATGATTA | |
| | TACATTTAAACAATTGAATTGAACTAGAAACTGGCCACATGGTTA | |
| | AAAGACATTTACAAATGTAATCATCCAGTGTTATGATGCCCAGAA | |
| | AAAAAAAATTCCTTAGAATGCTTTAAAAGCCGTATTCCATCACCT | |
| | TTCCAGT | |
| 455 | TAGCCGGACCCTTTGCCTTCGCCACTGCTCAGCGTCTGCACATCC | STMN2 |
| | CTACAATGGCTAAAACAGCAATGGGTAAGGCACTGCGCCTCGTTC | Exon 1 |
| | TCCGTCGGCTCTACCTGGAGCCCACCTCT | |
| 456 | AATCTTTCAAAACCTCCCTGGCTTAGAAAACCAAATTTTTGTAGA | STMN2 |
| | GAGAGATGGGTAGAATCTAATTTTATTCTAAAGCAATTAGCATTA | Exon 2 |
| | CATCATCACAGCAG | |
| 457 | GAGAAATTCAGAAAAAATGAAATATACTAATCTTCAGCTTTTCAT | STMN2 |
| | TTCAGCCTACAAGGAAAAAATGAAGGAGCTGTCCATGCTGTCACT | Exon 3 |
| | GATCTGCTCTTGCTTTTACCCGGAACCTCGCAACATCAACATCTA | |
| | TACTTACGATGGTGAGTAACCTAGGATAGACATACCCCTGCTAGC | |
| | TAGATCATTTGGAAAG | |
| 458 | CCATGCTGCAAGCTTTGTATGCTTAACATTCTCAAATGTTCACTT | STMN2 |
| | TTCAGATATGGAAGTGAAGCAAATCAACAAACGTGCCTCTGGCCA | Exon 4 |
| | GGCTTTTGAGCTGATCTTGAAGCCACCATCTCCTATCTCAGAAGC | |
| | CCCACGAACTTTAGCTTCTCCAAAGAAGAAAGACCTGTCCCTGGA | |
| | GGAGATCCAGAAGAAACTGGAGGCTGCAGAGGAAAGAAGAAAGGT | |
| | AACTTTTTCCATAGGTTTTCCTTCTCTCTCTCCCTCCCCTGCTCC | |
| | TCC | |
| 459 | CTAGGTTTGTGTTTGGATAATTATAAGATGGCTATGTTTTTCTTC | STMN2 |
| | CCCAGTCTCAGGAGGCCCAGGTGCTGAAACAATTGGCAGAGAAGA | Exon 5 |
| | GGGAACACGAGCGAGAAGTCCTTCAGAAGGCTTTGGAGGAGAACA | |
| | ACAACTTCAGCAAGATGGCGGAGGAAAAGCTGATCCTGAAAATGG | |
| | AACAAATTAAGGAAAACCGTGAGGCTAATCTAGCTGCTATTATTG | |
| | AACGTCTGCAGGAAAAGGTAATCTCAGCAGAGTCCTGAGCAGATG | |
| | GATATATTCATATGCAGCACAG | |
| 460 | TGTAGACTCCTTGAGATTAATAGAGTTTAACGATAAGTTTTACTT | STMN2 |
| | TATAGCTGGTCAAGTTTATTTCTTCTGAACTAAAAGAATCTATAG | Exon 6 |
| | AGTCTCAATTTCTGGAGCTTCAGAGGGAAGGAGAGAAGCAATGTA | |
| | AGCAACATTCTACAGAAATATAAATAATACTACTAATAATTAGCA | |
| | TC | |
| 461 | ACCAGACAAAAAGGGCCTGTGACATTTCTTCTTCCTTTTGTGTTT | STMN2 |
| | TTTAGGAGAGGCATGCTGCGGAGGTGCGCAGGAACAAGGAACTCC | Exon 7 |
| | AGGTTGAACTGTCTGGCTGAAGCAAGGGAGGGTCTGGCACGCCCC | |
| | ACCAATAGTAAATCCCCCTGCCTAT | |

In some embodiments, the gene editing system disclosed herein may comprise a Cas12i polypeptide as disclosed herein. In other embodiments, the gene editing system may comprise a nucleic acid encoding the Cas12i polypeptide. For example, the gene editing system may comprise a vector (e.g., a viral vector such as an AAV vector, such as AAV1, AAV2, AAV3, AAV4, AAV5, AAV6, AAV7, AAV8, AAV9, AAV10, AAV11 and AAV12) encoding the Cas12i polypeptide. Alternatively, the gene editing system may comprise a mRNA molecule encoding the Cas12i polypeptide. In some instances, the mRNA molecule may be codon-optimized.

### II. Preparation of Gene Editing System Components

The present disclosure provides methods for production of components of the gene editing systems disclosed herein, e.g., the RNA guide, methods for production of the Cas12i polypeptide, and methods for complexing the RNA guide and Cas12i polypeptide.

### A. RNA Guide

In some embodiments, the RNA guide is made by in vitro transcription of a DNA molecule. Thus, for example, in some embodiments, the RNA guide is generated by in vitro transcription of a DNA molecule encoding the RNA guide using an upstream promoter sequence (e.g., a T7 polymerase promoter sequence).

In some embodiments, the DNA molecule encodes multiple RNA guides or the in vitro transcription reaction includes multiple different DNA molecules, each encoding a different RNA guide. In some embodiments, the RNA guide is made using chemical synthetic methods. In some embodiments, the RNA guide is made by expressing the RNA guide sequence in cells transfected with a plasmid including sequences that encode the RNA guide. In some embodiments, the plasmid encodes multiple different RNA guides. In some embodiments, multiple different plasmids, each encoding a different RNA guide, are transfected into the cells. In some embodiments, the RNA guide is expressed from a plasmid that encodes the RNA guide and also encodes a Cas12i polypeptide. In some embodiments, the RNA guide is expressed from a plasmid that expresses the RNA guide but not a Cas12i polypeptide. In some embodiments, the RNA guide is purchased from a commercial vendor. In some embodiments, the RNA guide is synthesized using one or more modified nucleotide, e.g., as described above.

### B. Cas12i Polypeptide

In some embodiments, the Cas12i polypeptide of the present disclosure can be prepared by (a) culturing bacteria which produce the Cas12i polypeptide of the present disclosure, isolating the Cas12i polypeptide, optionally, purifying the Cas12i polypeptide, and complexing the Cas12i polypeptide with an RNA guide. The Cas12i polypeptide can be also prepared by (b) a known genetic engineering technique, specifically, by isolating a gene encoding the Cas12i polypeptide of the present disclosure from bacteria, constructing a recombinant expression vector, and then transferring the vector into an appropriate host cell that expresses the RNA guide for expression of a recombinant protein that complexes with the RNA guide in the host cell. Alternatively, the Cas12i polypeptide can be prepared by (c) an in vitro coupled transcription-translation system and then complexing with an RNA guide.

In some embodiments, a host cell is used to express the Cas12i polypeptide. The host cell is not particularly limited, and various known cells can be preferably used. Specific examples of the host cell include bacteria such as E. coli, yeasts (budding yeast, Saccharomyces cerevisiae, and fission yeast, Schizosaccharomyces pombe), nematodes (Caenorhabditis elegans), Xenopus laevis oocytes, and animal cells (for example, CHO cells, COS cells and HEK293 cells). The method for transferring the expression vector described above into host cells, i.e., the transformation method, is not particularly limited, and known methods such as electroporation, the calcium phosphate method, the liposome method and the DEAE dextran method can be used.

After a host is transformed with the expression vector, the host cells may be cultured, cultivated or bred, for production of the Cas12i polypeptide. After expression of the Cas12i polypeptide, the host cells can be collected and Cas12i polypeptide purified from the cultures etc. according to conventional methods (for example, filtration, centrifugation, cell disruption, gel filtration chromatography, ion exchange chromatography, etc.).

In some embodiments, the methods for Cas12i polypeptide expression comprises translation of at least 5 amino acids, at least 10 amino acids, at least 15 amino acids, at least 20 amino acids, at least 50 amino acids, at least 100 amino acids, at least 150 amino acids, at least 200 amino acids, at least 250 amino acids, at least 300 amino acids, at least 400 amino acids, at least 500 amino acids, at least 600 amino acids, at least 700 amino acids, at least 800 amino acids, at least 900 amino acids, or at least 1000 amino acids of the Cas12i polypeptide. In some embodiments, the methods for protein expression comprises translation of about 5 amino acids, about 10 amino acids, about 15 amino acids, about 20 amino acids, about 50 amino acids, about 100 amino acids, about 150 amino acids, about 200 amino acids, about 250 amino acids, about 300 amino acids, about 400 amino acids, about 500 amino acids, about 600 amino acids, about 700 amino acids, about 800 amino acids, about 900 amino acids, about 1000 amino acids or more of the Cas12i polypeptide.

A variety of methods can be used to determine the level of production of a Cas12i polypeptide in a host cell. Such methods include, but are not limited to, for example, methods that utilize either polyclonal or monoclonal antibodies specific for the Cas12i polypeptide or a labeling tag as described elsewhere herein. Exemplary methods include, but are not limited to, enzyme-linked immunosorbent assays (ELISA), radioimmunoassays (MA), fluorescent immunoassays (FIA), and fluorescent activated cell sorting (FACS). These and other assays are well known in the art (Sec, e.g., Maddox et al., J. Exp. Med. 158:1211 [1983]).

The present disclosure provides methods of in vivo expression of the Cas12i polypeptide in a cell, comprising providing a polyribonucleotide encoding the Cas12i polypeptide to a host cell wherein the polyribonucleotide encodes the Cas12i polypeptide, expressing the Cas12i polypeptide in the cell, and obtaining the Cas12i polypeptide from the cell.

The present disclosure further provides methods of in vivo expression of a Cas12i polypeptide in a cell, comprising providing a polyribonucleotide encoding the Cas12i polypeptide to a host cell wherein the polyribonucleotide encodes the Cas12i polypeptide and expressing the Cas12i polypeptide in the cell. In some embodiments, the polyribonucleotide encoding the Cas12i polypeptide is delivered to the cell with an RNA guide and, once expressed in the cell, the Cas12i polypeptide and the RNA guide form a complex. In some embodiments, the polyribonucleotide encoding the Cas12i polypeptide and the RNA guide are delivered to the cell within a single composition. In some embodiments, the polyribonucleotide encoding the Cas12i polypeptide and the RNA guide are comprised within separate compositions. In some embodiments, the host cell is present in a subject, e.g., a human patient.

### C. Complexes

In some embodiments, an RNA guide targeting STMN2 is complexed with a Cas12i polypeptide to form a ribonucleoprotein (RNP). In some embodiments, complexation of the RNA guide and Cas12i polypeptide occurs at a temperature lower than about any one of 20° C. 21° C. 22° C., 23° C., 24° C., 25° C., 26° C., 27° C., 28° C., 29° C., 30° C., 31° C., 32° C., 33° C., 34° C., 35° C., 36° C. 37° C., 38° C., 39° C., 40° C., 41° C., 42° C., 43° C., 44° C., 45° C., 50° C., or 55° C. In some embodiments, the RNA guide does not dissociate from the Cas12i polypeptide at about 37° C. over an incubation period of at least about any one of 10 mins, 15 mins, 20 mins, 25 mins, 30 mins, 35 mins, 40 mins, 45 mins, 50 mins, 55 mins, 1 hr, 2 hr, 3 hr, 4 hr, or more hours.

In some embodiments, the RNA guide and Cas12i polypeptide are complexed in a complexation buffer. In some embodiments, the Cas12i polypeptide is stored in a buffer that is replaced with a complexation buffer to form a complex with the RNA guide. In some embodiments, the Cas12i polypeptide is stored in a complexation buffer.

In some embodiments, the complexation buffer has a pH in a range of about 7.3 to 8.6. In one embodiment, the pH of the complexation buffer is about 7.3. In one embodiment, the pH of the complexation buffer is about 7.4. In one embodiment, the pH of the complexation buffer is about 7.5. In one embodiment, the pH of the complexation buffer is about 7.6. In one embodiment, the pH of the complexation buffer is about 7.7. In one embodiment, the pH of the complexation buffer is about 7.8. In one embodiment, the pH of the complexation buffer is about 7.9. In one embodiment, the pH of the complexation buffer is about 8.0. In one embodiment, the pH of the complexation buffer is about 8.1. In one embodiment, the pH of the complexation buffer is about 8.2. In one embodiment, the pH of the complexation buffer is about 8.3. In one embodiment, the pH of the complexation buffer is about 8.4. In one embodiment, the pH of the complexation buffer is about 8.5. In one embodiment, the pH of the complexation buffer is about 8.6.

In some embodiments, the Cas12i polypeptide can be overexpressed and complexed with the RNA guide in a host cell prior to purification as described herein. In some embodiments, mRNA or DNA encoding the Cas12i polypeptide is introduced into a cell so that the Cas12i polypeptide is expressed in the cell. In some embodiments, the RNA guide is also introduced into the cell, whether simultaneously, separately, or sequentially from a single mRNA or DNA construct, such that the RNP complex is formed in the cell.

### III. Genetic Editing Methods

The present disclosure also provides methods of modifying a target site within the STMN2 gene. In some embodiments, the methods comprise introducing a STMN2-targeting RNA guide and a Cas12i polypeptide into a cell. The STMN2-targeting RNA guide and Cas12i polypeptide can be introduced as a ribonucleoprotein complex into a cell. The STMN2-targeting RNA guide and Cas12i polypeptide can be introduced on a nucleic acid vector. The Cas12i polypeptide can be introduced as an mRNA. The RNA guide and template DNA can be introduced directly into the cell. In some embodiments, the composition described herein is delivered to a cell/tissue/person to reduce STMN2 in the cell/tissue/person. In some embodiments, the composition described herein is delivered to a cell/tissue/person to reduce STMN2 production in the cell/tissue/person. In some embodiments, the composition described herein is delivered to a cell/tissue/person to treat a neurodegenerative disease (e.g., amyotrophic lateral sclerosis (ALS) or frontotemporal dementia (FTD)) in a cell/tissue/person. In some embodiments, the composition described herein is delivered to a person with a neurodegenerative disease (e.g., amyotrophic lateral sclerosis (ALS) or frontotemporal dementia (FTD)).

Any of the gene editing systems disclosed herein may be used to genetically engineered a STMN2 gene. The gene editing system may comprise a guide RNA, a Cas12i2 polypeptide, and a template DNA. The guide RNA comprises a spacer sequence specific to a target sequence in the STMN2 gene, e.g., specific to a region in exon 1, exon 2, exon 3, exon 4, exon 5, exon 6, exon 7, or an intron of the STMN2 gene.

### A. Target Sequences

In some embodiments, an RNA guide as disclosed herein is designed to be complementary to a target sequence that is adjacent to a 5′-TTN-3′ PAM sequence or 5′-NTTN-3′ PAM sequence.

In some embodiments, the target sequence is within a STMN2 gene or a locus of a STMN2 gene (e.g., exon 1, exon 2, exon 3, exon 4, exon 5, exon 6, exon 7, or an intron), to which the RNA guide can bind via base pairing. In some embodiments, a cell has only one copy of the target sequence. In some embodiments, a cell has more than one copy, such as at least about any one of 2, 3, 4, 5, 10, 100, or more copies of the target sequence.

In some embodiments, the STMN2 gene is a mammalian gene. In some embodiments, the STMN2 gene is a human gene. For example, in some embodiments, the target sequence is within the sequence of SEQ ID NO: 454, or the reverse complement thereof. In some embodiments, the target sequence is within an exon of the STMN2 gene set forth in SEQ ID NO: 454, or the reverse complement thereof, e.g., within a sequence of any one of SEQ ID NOs: 455-461 (or a reverse complement of any thereof). Target sequences within an exon region of the STMN2 gene of SEQ ID NO: 454 are set forth in Table 6. The exon sequences are set forth in Table 7. In some embodiments, the target sequence is within an intron of the STMN2 gene set forth in SEQ ID NO: 454, or the reverse complement thereof. In some embodiments, the target sequence is within a variant (e.g., a polymorphic variant) of the STMN2 gene sequence set forth in SEQ ID NO: 454, or the reverse complement thereof. In some embodiments, the STMN2 gene sequence is a homolog of the sequence set forth in SEQ ID NO: 454, or the reverse complement thereof. In some embodiments, the STMN2 gene sequence is a non-human STMN2 sequence.

In some embodiments, the target sequence is adjacent to a 5′-NTTN-3′ PAM sequence, wherein N is any nucleotide. The 5′-NTTN-3′ sequence may be immediately adjacent to the target sequence or, for example, within a small number (e.g., 1, 2, 3, 4, or 5) of nucleotides of the target sequence. In some embodiments the 5′-NTTN-3′ sequence is 5′-NTTY-3′, 5′-NTTC-3′, 5′-NTTT-3′, 5′-NTTA-3′, 5′-NTTB-3′, 5′-NTTG-3′, 5′-CTTY-3′, 5′-DTTR′3′, 5′-CTTR-3′, 5′-DTTT-3′, 5′-ATTN-3′, or 5′-GTTN-3′, wherein Y is C or T. B is any nucleotide except for A. D is any nucleotide except for C, and R is A or G. In some embodiments, the 5′-NTTN-3′ sequence is 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′. The PAM sequence may be 5′ to the target sequence. In some embodiments, the target sequence is single-stranded (e.g., single-stranded DNA).

In some embodiments, the target sequence is double-stranded (e.g., double-stranded DNA). In some embodiments, the target sequence comprises both single-stranded and double-stranded regions. In some embodiments, the target sequence is linear. In some embodiments, the target sequence is circular. In some embodiments, the target sequence comprises one or more modified nucleotides, such as methylated nucleotides, damaged nucleotides, or nucleotides analogs. In some embodiments, the target sequence is not modified. In some embodiments, the RNA guide binds to a first strand of a double-stranded target sequence (e.g., the target strand or the spacer-complementary strand), and the 5′-NTTN-3′ PAM sequence is present in the second, complementary strand (e.g., the non-target strand or the non-spacer-complementary strand). In some embodiments, the RNA guide binds adjacent to a 5′-NAAN-3′ sequence on the target strand (e.g., the spacer-complementary strand).

The 5′-NTTN-3′ sequence may be immediately adjacent to the target sequence or, for example, within a small number (e.g., 1, 2, 3, 4, or 5) of nucleotides of the target sequence. In some embodiments the 5′-NTTN-3′ sequence is 5′-NTTY-3′, 5′-NTTC-3′, 5′-NTTT-3′, 5′-NTTA-3′, 5′-NTTB-3′, 5′-NTTG-3′, 5′-CTTY-3′, 5′-DTTR-3′, 5′-CTTR-3′, 5′-DTTT-3′, 5′-ATTN-3′, or 5′-GTTN-3′, wherein Y is C or T, B is any nucleotide except for A, D is any nucleotide except for C, and R is A or G. In some embodiments, the 5′-NTTN-3′ sequence is 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′. In some embodiments, the RNA guide is designed to bind to a first strand of a double-stranded target nucleic acid (i.e., the non-PAM strand), and the 5′-NTTN-3′ PAM sequence is present in the second, complementary strand (i.e., the PAM strand). In some embodiments, the RNA guide binds to a region on the non-PAM strand that is complementary to a target sequence on the PAM strand, which is adjacent to a 5′-NAAN-3′ sequence.

In some embodiments, the target sequence is present in a cell. In some embodiments, the target sequence is present in the nucleus of the cell. In some embodiments, the target sequence is endogenous to the cell. In some embodiments, the target sequence is a genomic DNA. In some embodiments, the target sequence is a chromosomal DNA. In some embodiments, the target sequence is a protein-coding gene or a functional region thereof, such as a coding region, or a regulatory element, such as a promoter, enhancer, a 5′ or 3′ untranslated region, etc. In some embodiments, the target sequence is present in a readily accessible region of the target sequence. In some embodiments, the target sequence is in an exon of a target gene. In some embodiments, the target sequence is across an exon-intron junction of a target gene. In some embodiments, the target sequence is present in a non-coding region, such as a regulatory region of a gene.

### B. Gene Editing

In some embodiments, the Cas12i polypeptide has enzymatic activity (e.g., nuclease activity). In some embodiments, the Cas12i polypeptide induces one or more DNA double-stranded breaks in the cell. In some embodiments, the Cas12i polypeptide induces one or more DNA single-stranded breaks in the cell. In some embodiments, the Cas12i polypeptide induces one or more DNA nicks in the cell. In some embodiments, DNA breaks and/or nicks result in formation of one or more indels (e.g., one or more deletions).

In some embodiments, an RNA guide disclosed herein forms a complex with the Cas12i polypeptide and directs the Cas12i polypeptide to a target sequence adjacent to a 5′-NTTN-3′ sequence. In some embodiments, the complex induces a deletion (e.g., a nucleotide deletion or DNA deletion) adjacent to the 5′-NTTN-3′ sequence. In some embodiments, the complex induces a deletion adjacent to a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the complex induces a deletion adjacent to a T/C-rich sequence.

In some embodiments, the deletion is downstream of a 5′-NTTN-3′ sequence. In some embodiments, the deletion is downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion is downstream of a T/C-rich sequence.

In some embodiments, the deletion alters expression of the STMN2 gene. In some embodiments, the deletion alters function of the STMN2 gene. In some embodiments, the deletion inactivates the STMN2 gene. In some embodiments, the deletion is a frameshifting deletion. In some embodiments, the deletion is a non-frameshifting deletion. In some embodiments, the deletion leads to cell toxicity or cell death (e.g., apoptosis).

In some embodiments, the deletion starts within about 5 to about 15 nucleotides (e.g., about 3, 4, 5, 6, 7, 8, 9, 10, 11, 12, 13, 14, 15, 16, or 17 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence.

In some embodiments, the deletion starts within about 5 to about 10 nucleotides (e.g., about 3, 4, 5, 6, 7, 8, 9, 10, 11, or 12 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence.

In some embodiments, the deletion starts within about 10 to about 15 nucleotides (e.g., about 8, 9, 10, 11, 12, 13, 14, 15, 16, or 17 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence.

In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) downstream of a T/C-rich sequence.

In some embodiments, the deletion ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion ends within about 20 to about 25 nucleotides (e.g., about 17, 18, 19, 20, 21, 22, 23, 24, 25, 26, 27, or 28 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 20 to about 25 nucleotides (e.g., about 17, 18, 19, 20, 21, 22, 23, 24, 25, 26, 27, or 28 nucleotides) of a T/C-rich sequence.

In some embodiments, the deletion ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion ends within about 20 to about 25 nucleotides (e.g., about 17, 18, 19, 20, 21, 22, 23, 24, 25, 26, 27, or 28 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 20 to about 25 nucleotides (e.g., about 17, 18, 19, 20, 21, 22, 23, 24, 25, 26, 27, or 28 nucleotides) downstream of a T/C-rich sequence.

In some embodiments, the deletion ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion ends within about 25 to about 30 nucleotides (e.g., about 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion ends within about 25 to about 30 nucleotides (e.g., about 22, 23, 24, 25, 26, 27, 28, 29, 30, 31, 32, or 33 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 25 to about 30 nucleotides (e.g., about 22, 23, 24, 25, 26, 27, 28, 29, 30, 31, 32, or 33 nucleotides) downstream of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence and ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 25 nucleotides (e.g., about 17, 18, 19, 20, 21, 22, 23, 24, 25, 26, 27, or 28 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence and ends within about 20 to about 25 nucleotides (e.g., about 17, 18, 19, 20, 21, 22, 23, 24, 25, 26, 27, or 28 nucleotides) downstream of the T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence and ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 25 to about 30 nucleotides (e.g., about 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence and ends within about 25 to about 30 nucleotides (e.g., about 22, 23, 24, 25, 26, 27, 28, 29, 30, 31, 32, or 33 nucleotides) downstream of the T/C-rich sequence.

In some embodiments, the deletion starts within about 5 to about 10 nucleotides (e.g., about 3, 4, 5, 6, 7, 8, 9, 10, 11, or 12 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the T/C-rich sequence.

In some embodiments, the deletion starts within about 5 to about 10 nucleotides (e.g., about 3, 4, 5, 6, 7, 8, 9, 10, 11, or 12 nucleotides) and ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) and ends within about 20 to about 25 nucleotides (e.g., about 17, 18, 19, 20, 21, 22, 23, 24, 25, 26, 27, or 28 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides and ends within about 20 to about 25 nucleotides (e.g., about 17, 18, 19, 20, 21, 22, 23, 24, 25, 26, 27, or 28 nucleotides) (e.g., about 3, 4, 5, 6, 7, 8, 9, 10, 11, or 12 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence and ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 25 nucleotides (e.g., about 17, 18, 19, 20, 21, 22, 23, 24, 25, 26, 27, or 28 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence and ends within about 20 to about 25 nucleotides (e.g., about 17, 18, 19, 20, 21, 22, 23, 24, 25, 26, 27, or 28 nucleotides) downstream of the T/C-rich sequence.

In some embodiments, the deletion starts within about 5 to about 10 nucleotides (e.g., about 3, 4, 5, 6, 7, 8, 9, 10, 11, or 12 nucleotides) and ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) and ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence and ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 25 to about 30 nucleotides (e.g., about 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence and ends within about 25 to about 30 nucleotides (e.g., about 22, 23, 24, 25, 26, 27, 28, 29, 30, 31, 32, or 33 nucleotides) downstream of the T/C-rich sequence.

In some embodiments, the deletion starts within about 10 to about 15 nucleotides (e.g., about 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the T/C-rich sequence.

In some embodiments, the deletion starts within about 10 to about 15 nucleotides (e.g., about 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence and ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 25 nucleotides (e.g., about 17, 18, 19, 20, 21, 22, 23, 24, 25, 26, 27, or 28 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence and ends within about 20 to about 25 nucleotides (e.g., about 17, 18, 19, 20, 21, 22, 23, 24, 25, 26, 27, or 28 nucleotides) downstream of the T/C-rich sequence.

In some embodiments, the deletion starts within about 10 to about 15 nucleotides (e.g., about 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence and ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-ATTA-3′, 5′-ATTT-3′, 5′-ATTG-3′, 5′-ATTC-3′. 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 25 to about 30 nucleotides (e.g., about 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence and ends within about 25 to about 30 nucleotides (e.g., about 22, 23, 24, 25, 26, 27, 28, 29, 30, 31, 32, or 33 nucleotides) downstream of the T/C-rich sequence.

In some embodiments, the deletion is up to about 50 nucleotides in length (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 nucleotides). In some embodiments, the deletion is up to about 40 nucleotides in length (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides). In some embodiments, the deletion is between about 4 nucleotides and about 40 nucleotides in length (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides). In some embodiments, the deletion is between about 4 nucleotides and about 25 nucleotides in length (e.g., about 3, 4, 5, 6, 7, 8, 9, 10, 11, 12, 13, 14, 15, 16, 17, 18, 19, 20, 21, 22, 23, 24, 25, 26, 27, or 28 nucleotides). In some embodiments, the deletion is between about 10 nucleotides and about 25 nucleotides in length (e.g., about 7, 8, 9, 10, 11, 12, 13, 14, 15, 16, 17, 18, 19, 20, 21, 22, 23, 24, 25, 26, 27, or 28 nucleotides). In some embodiments, the deletion is between about 10 nucleotides and about 15 nucleotides in length (e.g., about 7, 8, 9, 10, 11, 12, 13, 14, 15, 16, or 17 nucleotides).

In some embodiments, two or more RNA guides described herein are used to introduce a deletion that has a length of greater than 40 nucleotides. In some embodiments, two or more RNA guides described herein are used to introduce a deletion of at least about 41, 42, 43, 44, 45, 46, 47, 48, 49, 50, 55, 60, 65, 70, 75, 80, 85, 90, 95, 100, 110, 120, 130, 140, 150, 16, 170, 180, 190, 200, 210, 220, 230, 240, 250, 260, 270, 280, 290, 300, 310, 320, 330, 340, 350, 360, 370, 380, 390, or 400 nucleotides. In some embodiments, two or more RNA guides described herein are used delete all or a portion of the STMN2 gene or SEQ ID NO: 454.

In some embodiments, the methods described herein are used to engineer a cell comprising a deletion as described herein in a STMN2 gene. In some embodiments, the methods are carried out using a complex comprising a Cas12i enzyme as described herein and an RNA guide comprising a direct repeat sequence and a spacer sequence as described herein.

In some embodiments, the RNA guide targeting STMN2 is encoded in a plasmid. In some embodiments, the RNA guide targeting STMN2 is synthetic or purified RNA. In some embodiments, the Cas12i polypeptide is encoded in a plasmid. In some embodiments, the Cas12i polypeptide is encoded by an RNA that is synthetic or purified.

### C. Delivery

Components of any of the gene editing systems disclosed herein may be formulated, for example, including a carrier, such as a carrier and/or a polymeric carrier, e.g., a liposome, and delivered by known methods to a cell (e.g., a prokaryotic, eukaryotic, plant, mammalian, etc.). Such methods include, but not limited to, transfection (e.g., lipid-mediated, cationic polymers, calcium phosphate, dendrimers); electroporation or other methods of membrane disruption (e.g., nucleofection), viral delivery (e.g., lentivirus, retrovirus, adenovirus, adeno-associated virus (AAV)), microinjection, microprojectile bombardment (“gene gun”), fugene, direct sonic loading, cell squeezing, optical transfection, protoplast fusion, impalefection, magnetofection, exosome-mediated transfer, lipid nanoparticle-mediated transfer, and any combination thereof.

In some embodiments, the method comprises delivering one or more nucleic acids (e.g., nucleic acids encoding the Cas12i polypeptide, RNA guide, donor DNA, etc.), one or more transcripts thereof, and/or a pre-formed RNA guide/Cas12i polypeptide complex to a cell, where a ternary complex is formed. In some embodiments, an RNA guide and an RNA encoding a Cas12i polypeptide are delivered together in a single composition. In some embodiments, an RNA guide and an RNA encoding a Cas12i polypeptide are delivered in separate compositions. In some embodiments, an RNA guide and an RNA encoding a Cas12i polypeptide delivered in separate compositions are delivered using the same delivery technology. In some embodiments, an RNA guide and an RNA encoding a Cas12i polypeptide delivered in separate compositions are delivered using different delivery technologies.

In some embodiments, the Cas12i component and the RNA guide component are delivered together. For example, the Cas12i component and the RNA guide component are packaged together in a single AAV particle. In another example, the Cas12i component and the RNA guide component are delivered together via lipid nanoparticles (LNPs). In some embodiments, the Cas12i component and the RNA guide component are delivered separately. For example, the Cas12i component and the RNA guide are packaged into separate AAV particles. In another example, the Cas12i component is delivered by a first delivery mechanism and the RNA guide is delivered by a second delivery mechanism.

Exemplary intracellular delivery methods, include, but are not limited to: viruses, such as AAV, or virus-like agents; chemical-based transfection methods, such as those using calcium phosphate, dendrimers, liposomes, or cationic polymers (e.g., DEAE-dextran or polyethylenimine); non-chemical methods, such as microinjection, electroporation, cell squeezing, sonoporation, optical transfection, impalefection, protoplast fusion, bacterial conjugation, delivery of plasmids or transposons; particle-based methods, such as using a gene gun, magnetofection or magnet assisted transfection, particle bombardment; and hybrid methods, such as nucleofection. In some embodiments, a lipid nanoparticle comprises an mRNA encoding a Cas12i polypeptide, an RNA guide, or an mRNA encoding a Cas12i polypeptide and an RNA guide. In some embodiments, the mRNA encoding the Cas12i polypeptide is a transcript of the nucleotide sequence set forth in SEQ ID NO: 447 or SEQ ID NO: 481 or a variant thereof. In some embodiments, the present application further provides cells produced by such methods, and organisms (such as animals, plants, or fungi) comprising or produced from such cells.

### D. Genetically Modified Cells

Any of the gene editing systems disclosed herein can be delivered to a variety of cells. In some embodiments, the cell is an isolated cell. In some embodiments, the cell is in cell culture or a co-culture of two or more cell types. In some embodiments, the cell is ex vivo. In some embodiments, the cell is obtained from a living organism and maintained in a cell culture. In some embodiments, the cell is a single-cellular organism.

In some embodiments, the cell is a prokaryotic cell. In some embodiments, the cell is a bacterial cell or derived from a bacterial cell. In some embodiments, the cell is an archaeal cell or derived from an archaeal cell.

In some embodiments, the cell is a eukaryotic cell. In some embodiments, the cell is a plant cell or derived from a plant cell. In some embodiments, the cell is a fungal cell or derived from a fungal cell. In some embodiments, the cell is an animal cell or derived from an animal cell. In some embodiments, the cell is an invertebrate cell or derived from an invertebrate cell. In some embodiments, the cell is a vertebrate cell or derived from a vertebrate cell. In some embodiments, the cell is a mammalian cell or derived from a mammalian cell. In some embodiments, the cell is a human cell. In some embodiments, the cell is a zebra fish cell. In some embodiments, the cell is a rodent cell. In some embodiments, the cell is synthetically made, sometimes termed an artificial cell.

In some embodiments, the cell is derived from a cell line. A wide variety of cell lines for tissue culture are known in the art. Examples of cell lines include, but are not limited to, 293T. MF7, K562, HeLa, CHO, and transgenic varieties thereof. Cell lines are available from a variety of sources known to those with skill in the art (see, e.g., the American Type Culture Collection (ATCC) (Manassas, Va.)). In some embodiments, the cell is an immortal or immortalized cell.

In some embodiments, the cell is a primary cell. In some embodiments, the cell is a stem cell such as a totipotent stem cell (e.g., omnipotent), a pluripotent stem cell, a multipotent stem cell, an oligopotent stem cell, or an unipotent stem cell. In some embodiments, the cell is an induced pluripotent stem cell (iPSC) or derived from an iPSC. In some embodiments, the cell is a differentiated cell. For example, in some embodiments, the differentiated cell is a neural cell (e.g., a glial cell, such as an astrocyte, an oligodendrocyte, a microglial cell, or an ependymal cell, or a neuron), muscle cell (e.g., a myocyte), a fat cell (e.g., an adipocyte), a bone cell (e.g., an osteoblast, osteocyte, osteoclast), a blood cell (e.g., a monocyte, a lymphocyte, a neutrophil, an eosinophil, a basophil, a macrophage, a erythrocyte, or a platelet), an epithelial cell, an immune cell (e.g., a lymphocyte, a neutrophil, a monocyte, or a macrophage), a liver cell (e.g., a hepatocyte), a fibroblast, or a sex cell. In some embodiments, the cell is a terminally differentiated cell. For example, in some embodiments, the terminally differentiated cell is a neuronal cell, an adipocyte, a cardiomyocyte, a skeletal muscle cell, an epidermal cell, or a gut cell. In some embodiments, the cell is an immune cell. In some embodiments, the immune cell is a T cell. In some embodiments, the immune cell is a B cell. In some embodiments, the immune cell is a Natural Killer (NK) cell. In some embodiments, the immune cell is a Tumor Infiltrating Lymphocyte (TIL). In some embodiments, the cell is a cancer cell (e.g., a colorectal cancer cell, renal cell cancer cell, breast cancer cell, or glioma cell). In some embodiments, the cell is a mammalian cell, e.g., a human cell or a murine cell. In some embodiments, the murine cell is derived from a wild-type mouse, an immunosuppressed mouse, or a disease-specific mouse model. In some embodiments, the cell is a cell within a living tissue, organ, or organism.

Any of the genetically modified cells produced using any of the gene editing system disclosed herein is also within the scope of the present disclosure. Such modified cells may comprise a disrupted STMN2 gene.

Any of the gene editing systems, compositions comprising such, vectors, nucleic acids, RNA guides and cells disclosed herein may be used in therapy. Gene editing systems, compositions, vectors, nucleic acids, RNA guides and cells disclosed herein may be used in methods of treating a disease or condition in a subject. Any suitable delivery or administration method known in the art may be used to deliver compositions, vectors, nucleic acids, RNA guides and cells disclosed herein. Such methods may involve contacting a target sequence with a composition, vector, nucleic acid, or RNA guide disclosed herein. Such methods may involve a method of editing a STMN2 sequence as disclosed herein. In some embodiments, a cell engineered using an RNA guide disclosed herein is used for ex vivo gene therapy.

### IV. Therapeutic Applications

Any of the gene editing systems or modified cells generated using such a gene editing system as disclosed herein may be used for treating a disease that is associated with the STMN2 gene, for example, neurodegenerative diseases (e.g., amyotrophic lateral sclerosis (ALS) or frontotemporal dementia (FTD)). Any suitable delivery or administration method known in the art may be used to deliver compositions, vectors, nucleic acids, RNA guides and cells disclosed herein. Such methods may involve contacting a target sequence with a composition, vector, nucleic acid, or RNA guide disclosed herein. Such methods may involve a method of editing a STMN2 sequence as disclosed herein. In some embodiments, a cell engineered using an RNA guide disclosed herein is used for ex vivo gene therapy. In some embodiments, provided herein is a method for treating a target disease as disclosed herein (e.g., a neurodegenerative disease) comprising administering to a subject (e.g., a human patient) in need of the treatment any of the gene editing systems disclosed herein. The gene editing system may be delivered to a specific tissue or specific type of cells where the gene edit is needed. The gene editing system may comprise LNPs encompassing one or more of the components, one or more vectors (e.g., viral vectors) encoding one or more of the components, or a combination thereof. Components of the gene editing system may be formulated to form a pharmaceutical composition, which may further comprise one or more pharmaceutically acceptable carriers.

In some embodiments, modified cells produced using any of the gene editing systems disclosed herein may be administered to a subject (e.g., a human patient) in need of the treatment. The modified cells may comprise a substitution, insertion, and/or deletion described herein. In some examples, the modified cells may include a cell line modified by a CRISPR nuclease, reverse transcriptase polypeptide, and editing template RNA (e.g., RNA guide and RT donor RNA). In some instances, the modified cells may be a heterogenous population comprising cells with different types of gene edits. Alternatively, the modified cells may comprise a substantially homogenous cell population (e.g., at least 80% of the cells in the whole population) comprising one particular gene edit in the STMN2 gene. In some examples, the cells can be suspended in a suitable media.

In some embodiments, provided herein is a composition comprising the gene editing system or components thereof. Such a composition can be a pharmaceutical composition. A pharmaceutical composition that is useful may be prepared, packaged, or sold in a formulation suitable for oral, rectal, vaginal, parenteral, topical, pulmonary, intranasal, intra-lesional, buccal, ophthalmic, intravenous, intra-organ or another route of administration. A pharmaceutical composition of the disclosure may be prepared, packaged, or sold in bulk, as a single unit dose, or as a plurality of single unit doses. As used herein, a “unit dose” is discrete amount of the pharmaceutical composition (e.g., the gene editing system or components thereof), which would be administered to a subject or a convenient fraction of such a dosage such as, for example, one-half or one-third of such a dosage.

A formulation of a pharmaceutical composition suitable for parenteral administration may comprise the active agent (e.g., the gene editing system or components thereof or the modified cells) combined with a pharmaceutically acceptable carrier, such as sterile water or sterile isotonic saline. Such a formulation may be prepared, packaged, or sold in a form suitable for bolus administration or for continuous administration. Some injectable formulations may be prepared, packaged, or sold in unit dosage form, such as in ampules or in multi-dose containers containing a preservative. Some formulations for parenteral administration include, but are not limited to, suspensions, solutions, emulsions in oily or aqueous vehicles, pastes, and implantable sustained-release or biodegradable formulations. Some formulations may further comprise one or more additional ingredients including, but not limited to, suspending, stabilizing, or dispersing agents.

The pharmaceutical composition may be in the form of a sterile injectable aqueous or oily suspension or solution. This suspension or solution may be formulated according to the known art, and may comprise, in addition to the cells, additional ingredients such as the dispersing agents, wetting agents, or suspending agents described herein. Such sterile injectable formulation may be prepared using a non-toxic parenterally-acceptable diluent or solvent, such as water or saline. Other acceptable diluents and solvents include, but are not limited to, Ringer's solution, isotonic sodium chloride solution, and fixed oils such as synthetic mono- or di-glycerides. Other parentally-administrable formulations which that are useful include those which may comprise the cells in a packaged form, in a liposomal preparation, or as a component of a biodegradable polymer system. Some compositions for sustained release or implantation may comprise pharmaceutically acceptable polymeric or hydrophobic materials such as an emulsion, an ion exchange resin, a sparingly soluble polymer, or a sparingly soluble salt.

### V. Kits and Uses Thereof

The present disclosure also provides kits that can be used, for example, to carry out a method described herein for genetical modification of the STMN2 gene. In some embodiments, the kits include an RNA guide and a Cas12i polypeptide. In some embodiments, the kits include an RNA guide, a template DNA, and a Cas12i polypeptide. In some embodiments, the kits include a polynucleotide that encodes such a Cas12i polypeptide, and optionally the polynucleotide is comprised within a vector, e.g., as described herein. In some embodiments, the kits include a polynucleotide that encodes an RNA guide disclosed herein. The Cas12i polypeptide (or polynucleotide encoding the Cas12i polypeptide) and the RNA guide (e.g., as a ribonucleoprotein) can be packaged within the same or other vessel within a kit or can be packaged in separate vials or other vessels, the contents of which can be mixed prior to use.

The Cas12i polypeptide, the RNA guide, and the template DNA can be packaged within the same or other vessel within a kit or can be packaged in separate vials or other vessels, the contents of which can be mixed prior to use. The kits can additionally include, optionally, a buffer and/or instructions for use of the RNA guide, template DNA, and Cas12i polypeptide.

All references and publications cited herein are hereby incorporated by reference.

### Additional Embodiments

Provided below are additional embodiments, which are also within the scope of the present disclosure.

Embodiment 1: A composition comprising an RNA guide, wherein the RNA guide comprises (i) a spacer sequence that is substantially complementary or complete complementary to a region on a non-PAM strand (the complementary sequence of a target sequence) within an STMN2 gene and (ii) a direct repeat sequence; wherein the target sequence is adjacent to a protospacer adjacent motif (PAM) comprising the sequence 5′-NTTN-3′.

In Embodiment 1, the target sequence may be within exon 1, exon 2, exon 3, exon 4, exon 5, exon 6, exon 7, or an intron of the STMN2 gene. In some examples, the STMN2 gene comprises the sequence of SEQ ID NO: 454, the reverse complement of SEQ ID NO: 454, a variant of SEQ ID NO: 454, or the reverse complement of a variant of SEQ ID NO: 454.

In Embodiment 1, the spacer sequence may comprise: (a) nucleotide 1 through nucleotide 16 of a sequence that is at least 90% identical to a sequence of any one of SEQ ID NOs: 229-446 or 2497-4502; (b) nucleotide 1 through nucleotide 17 of a sequence that is at least 90% identical to a sequence of any one of SEQ ID NOs: 229-446 or 2497-4502; (c) nucleotide 1 through nucleotide 18 of a sequence that is at least 90% identical to a sequence of any one of SEQ ID NOs: 229-446 or 2497-4502; (d) nucleotide 1 through nucleotide 19 of a sequence that is at least 90% identical to a sequence of any one of SEQ ID NOs: 229-446 or 2497-4502; I nucleotide 1 through nucleotide 20 of a sequence that is at least 90% identical to a sequence of any one of SEQ ID NOs: 229-446 or 2497-4502; (f) nucleotide 1 through nucleotide 21 of a sequence that is at least 90% identical to a sequence of any one of SEQ ID NOs: 229-446 or 2497-4502; (g) nucleotide 1 through nucleotide 22 of a sequence that is at least 90% identical to a sequence of any one of SEQ ID NOs: 229-446 or 2497-4502; (h) nucleotide 1 through nucleotide 23 of a sequence that is at least 90% identical to a sequence of any one of SEQ ID NOs: 229-446 or 2497-4502; (i) nucleotide 1 through nucleotide 24 of a sequence that is at least 90% identical to a sequence of any one of SEQ ID NOs: 229-446 or 2497-4502; (j) nucleotide 1 through nucleotide 25 of a sequence that is at least 90% identical to a sequence of any one of SEQ ID NOs: 229-446 or 2497-4502; (k) nucleotide 1 through nucleotide 26 of a sequence that is at least 90% identical to a sequence of any one of SEQ ID NOs: 229-446 or 2497-4502; (1) nucleotide 1 through nucleotide 27 of a sequence that is at least 90% identical to a sequence of any one of SEQ ID NOs: 229-446 or 2497-4502; (m) nucleotide 1 through nucleotide 28 of a sequence that is at least 90% identical to a sequence of any one of SEQ ID NOs: 229-446 or 2497-4502; (n) nucleotide 1 through nucleotide 29 of a sequence that is at least 90% identical to a sequence of any one of SEQ ID NOs: 229-446 or 2497-4502; or (o) nucleotide 1 through nucleotide 30 of a sequence that is at least 90% identical to a sequence of any one of SEQ ID NOs: 229-446 or 2497-4502.

In any of the compositions of Embodiment 1, the spacer sequence may comprise: (a) nucleotide 1 through nucleotide 16 of any one of SEQ ID NOs: 229-446 or 2497-4502; (b) nucleotide 1 through nucleotide 17 of any one of SEQ ID NOs: 229-446 or 2497-4502; (c) nucleotide 1 through nucleotide 18 of any one of SEQ ID NOs: 229-446 or 2497-4502; (d) nucleotide 1 through nucleotide 19 of any one of SEQ ID NOs: 229-446 or 2497-4502; (c) nucleotide 1 through nucleotide 20 of any one of SEQ ID NOs: 229-446 or 2497-4502; (f) nucleotide 1 through nucleotide 21 of any one of SEQ ID NOs: 229-446 or 2497-4502; (g) nucleotide 1 through nucleotide 22 of any one of SEQ ID NOs: 229-446 or 2497-4502; (h) nucleotide 1 through nucleotide 23 of any one of SEQ ID NOs: 229-446 or 2497-4502; (i) nucleotide 1 through nucleotide 24 of any one of SEQ ID NOs: 229-446 or 2497-4502; (j) nucleotide 1 through nucleotide 25 of any one of SEQ ID NOs: 229-446 or 2497-4502; (k) nucleotide 1 through nucleotide 26 of any one of SEQ ID NOs: 229-446 or 2497-4502; (l) nucleotide 1 through nucleotide 27 of any one of SEQ ID NOs: 229-446 or 2497-4502; (m) nucleotide 1 through nucleotide 28 of any one of SEQ ID NOs: 229-446 or 2497-4502; (n) nucleotide 1 through nucleotide 29 of any one of SEQ ID NOs: 229-446 or 2497-4502; or (0) nucleotide 1 through nucleotide 30 of any one of SEQ ID NOs: 229-446 or 2497-4502.

In any of the compositions of Embodiment 1, the direct repeat sequence may comprise: (a) nucleotide 1 through nucleotide 36 of a sequence that is at least 90% identical to a sequence of any one of SEQ ID NOs: 1-8; (b) nucleotide 2 through nucleotide 36 of a sequence that is at least 90% identical to a sequence of any one of SEQ ID NOs: 1-8; (c) nucleotide 3 through nucleotide 36 of a sequence that is at least 90% identical to a sequence of any one of SEQ ID NOs: 1-8; (d) nucleotide 4 through nucleotide 36 of a sequence that is at least 90% identical to a sequence of any one of SEQ ID NOs: 1-8; (e) nucleotide 5 through nucleotide 36 of a sequence that is at least 90% identical to a sequence of any one of SEQ ID NOs: 1-8; (f) nucleotide 6 through nucleotide 36 of a sequence that is at least 90% identical to a sequence of any one of SEQ ID NOs: 1-8; (g) nucleotide 7 through nucleotide 36 of a sequence that is at least 90% identical to a sequence of any one of SEQ ID NOs: 1-8; (h) nucleotide 8 through nucleotide 36 of a sequence that is at least 90% identical to a sequence of any one of SEQ ID NOs: 1-8; (i) nucleotide 9 through nucleotide 36 of a sequence that is at least 90% identical to a sequence of any one of SEQ ID NOs: 1-8; (j) nucleotide 10 through nucleotide 36 of a sequence that is at least 90% identical to a sequence of any one of SEQ ID NOs: 1-8; (k) nucleotide 11 through nucleotide 36 of a sequence that is at least 90% identical to a sequence of any one of SEQ ID NOs: 1-8; (1) nucleotide 12 through nucleotide 36 of a sequence that is at least 90% identical to a sequence of any one of SEQ ID NOs: 1-8; (m) nucleotide 13 through nucleotide 36 of a sequence that is at least 90% identical to a sequence of any one of SEQ ID NOs: 1-8; (n) nucleotide 14 through nucleotide 36 of a sequence that is at least 90% identical to a sequence of any one of SEQ ID NOs: 1-8; (o) nucleotide 1 through nucleotide 34 of a sequence that is at least 90% identical to a sequence of SEQ ID NO: 9; (p) nucleotide 2 through nucleotide 34 of a sequence that is at least 90% identical to a sequence of SEQ ID NO: 9; (q) nucleotide 3 through nucleotide 34 of a sequence that is at least 90% identical to a sequence of SEQ ID NO: 9; (r) nucleotide 4 through nucleotide 34 of a sequence that is at least 90% identical to a sequence of SEQ ID NO: 9; (s) nucleotide 5 through nucleotide 34 of a sequence that is at least 90% identical to a sequence of SEQ ID NO: 9; (t) nucleotide 6 through nucleotide 34 of a sequence that is at least 90% identical to a sequence of SEQ ID NO: 9; (u) nucleotide 7 through nucleotide 34 of a sequence that is at least 90% identical to a sequence of SEQ ID NO: 9; (v) nucleotide 8 through nucleotide 34 of a sequence that is at least 90% identical to a sequence of SEQ ID NO: 9; (w) nucleotide 9 through nucleotide 34 of a sequence that is at least 90% identical to a sequence of SEQ ID NO: 9; (x) nucleotide 10 through nucleotide 34 of a sequence that is at least 90% identical to a sequence of SEQ ID NO: 9; (y) nucleotide 11 through nucleotide 34 of a sequence that is at least 90% identical to a sequence of SEQ ID NO: 9; (z) nucleotide 12 through nucleotide 34 of a sequence that is at least 90% identical to a sequence of SEQ ID NO: 9; or (aa) a sequence that is at least 90% identical to a sequence of SEQ ID NO: 10 or a portion thereof.

In some examples, the direct repeat sequence comprises: (a) nucleotide 1 through nucleotide 36 of any one of SEQ ID NOs: 1-8; (b) nucleotide 2 through nucleotide 36 of any one of SEQ ID NOs: 1-8; (c) nucleotide 3 through nucleotide 36 of any one of SEQ ID NOs: 1-8; (d) nucleotide 4 through nucleotide 36 of any one of SEQ ID NOs: 1-8; (e) nucleotide 5 through nucleotide 36 of any one of SEQ ID NOs: 1-8; (f) nucleotide 6 through nucleotide 36 of any one of SEQ ID NOs: 1-8; (g) nucleotide 7 through nucleotide 36 of any one of SEQ ID NOs: 1-8; (h) nucleotide 8 through nucleotide 36 of any one of SEQ ID NOs: 1-8; (i) nucleotide 9 through nucleotide 36 of any one of SEQ ID NOs: 1-8; (j) nucleotide 10 through nucleotide 36 of any one of SEQ ID NOs: 1-8; (k) nucleotide 11 through nucleotide 36 of any one of SEQ ID NOs: 1-8; (1) nucleotide 12 through nucleotide 36 of any one of SEQ ID NOs: 1-8; (m) nucleotide 13 through nucleotide 36 of any one of SEQ ID NOs: 1-8; (n) nucleotide 14 through nucleotide 36 of any one of SEQ ID NOs: 1-8; (0) nucleotide 1 through nucleotide 34 of SEQ ID NO: 9; (p) nucleotide 2 through nucleotide 34 of SEQ ID NO: 9; (q) nucleotide 3 through nucleotide 34 of SEQ ID NO: 9; (r) nucleotide 4 through nucleotide 34 of SEQ ID NO: 9; (s) nucleotide 5 through nucleotide 34 of SEQ ID NO: 9; (t) nucleotide 6 through nucleotide 34 of SEQ ID NO: 9; (u) nucleotide 7 through nucleotide 34 of SEQ ID NO: 9; (v) nucleotide 8 through nucleotide 34 of SEQ ID NO: 9; (w) nucleotide 9 through nucleotide 34 of SEQ ID NO: 9; (x) nucleotide 10 through nucleotide 34 of SEQ ID NO: 9; (y) nucleotide 11 through nucleotide 34 of SEQ ID NO: 9; (z) nucleotide 12 through nucleotide 34 of SEQ ID NO: 9; or (aa) SEQ ID NO: 10 or a portion thereof.

In some examples, the direct repeat sequence comprises: (a) nucleotide 1 through nucleotide 36 of a sequence that is at least 90% identical to a sequence of any one of SEQ ID NOs: 462-479; (b) nucleotide 2 through nucleotide 36 of a sequence that is at least 90% identical to a sequence of any one of SEQ ID NOs: 462-479; (c) nucleotide 3 through nucleotide 36 of a sequence that is at least 90% identical to a sequence of any one of SEQ ID NOs: 462-479; (d) nucleotide 4 through nucleotide 36 of a sequence that is at least 90% identical to a sequence of any one of SEQ ID NOs: 462-479; (c) nucleotide 5 through nucleotide 36 of a sequence that is at least 90% identical to a sequence of any one of SEQ ID NOs: 462-479; (f) nucleotide 6 through nucleotide 36 of a sequence that is at least 90% identical to a sequence of any one of SEQ ID NOs: 462-479; (g) nucleotide 7 through nucleotide 36 of a sequence that is at least 90% identical to a sequence of any one of SEQ ID NOs: 462-479; (h) nucleotide 8 through nucleotide 36 of a sequence that is at least 90% identical to a sequence of any one of SEQ ID NOs: 462-479; (i) nucleotide 9 through nucleotide 36 of a sequence that is at least 90% identical to a sequence of any one of SEQ ID NOs: 462-479; (j) nucleotide 10 through nucleotide 36 of a sequence that is at least 90% identical to a sequence of any one of SEQ ID NOs: 462-479; (k) nucleotide 11 through nucleotide 36 of a sequence that is at least 90% identical to a sequence of any one of SEQ ID NOs: 462-479; (1) nucleotide 12 through nucleotide 36 of a sequence that is at least 90% identical to a sequence of any one of SEQ ID NOs: 462-479; (m) nucleotide 13 through nucleotide 36 of a sequence that is at least 90% identical to a sequence of any one of SEQ ID NOs: 462-479; (n) nucleotide 14 through nucleotide 36 of a sequence that is at least 90% identical to a sequence of any one of SEQ ID NOs: 462-479; or (o) a sequence that is at least 90% identical to a sequence of SEQ ID NO: 480 or a portion thereof.

In some examples, the direct repeat sequence comprises: (a) nucleotide 1 through nucleotide 36 of any one of SEQ ID NOs: 462-479; (b) nucleotide 2 through nucleotide 36 of any one of SEQ ID NOs: 462-479; (c) nucleotide 3 through nucleotide 36 of any one of SEQ ID NOs: 462-479; (d) nucleotide 4 through nucleotide 36 of any one of SEQ ID NOs: 462-479; (c) nucleotide 5 through nucleotide 36 of any one of SEQ ID NOs: 462-479; (f) nucleotide 6 through nucleotide 36 of any one of SEQ ID NOs: 462-479; (g) nucleotide 7 through nucleotide 36 of any one of SEQ ID NOs: 462-479; (h) nucleotide 8 through nucleotide 36 of any one of SEQ ID NOs: 462-479; (i) nucleotide 9 through nucleotide 36 of any one of SEQ ID NOs: 462-479; (j) nucleotide 10 through nucleotide 36 of any one of SEQ ID NOs: 462-479; (k) nucleotide 11 through nucleotide 36 of any one of SEQ ID NOs: 462-479; (1) nucleotide 12 through nucleotide 36 of any one of SEQ ID NOs: 462-479; (m) nucleotide 13 through nucleotide 36 of any one of SEQ ID NOs: 462-479; (n) nucleotide 14 through nucleotide 36 of any one of SEQ ID NOs: 462-479; or (o) SEQ ID NO: 480 or a portion thereof.

In some examples, the direct repeat sequence comprises: (a) nucleotide 1 through nucleotide 36 of a sequence that is at least 90% identical to SEQ ID NO: 485; (b) nucleotide 2 through nucleotide 36 of a sequence that is at least 90% identical to SEQ ID NO: 485; (c) nucleotide 3 through nucleotide 36 of a sequence that is at least 90% identical to SEQ ID NO: 485; (d) nucleotide 4 through nucleotide 36 of a sequence that is at least 90% identical to SEQ ID NO: 485; (c) nucleotide 5 through nucleotide 36 of a sequence that is at least 90% identical to SEQ ID NO: 485; (f) nucleotide 6 through nucleotide 36 of a sequence that is at least 90% identical to SEQ ID NO: 485; (g) nucleotide 7 through nucleotide 36 of a sequence that is at least 90% identical to SEQ ID NO: 485; (h) nucleotide 8 through nucleotide 36 of a sequence that is at least 90% identical to SEQ ID NO: 485; (i) nucleotide 9 through nucleotide 36 of a sequence that is at least 90% identical to SEQ ID NO: 485; (j) nucleotide 10 through nucleotide 36 of a sequence that is at least 90% identical to SEQ ID NO: 485; (k) nucleotide 11 through nucleotide 36 of a sequence that is at least 90% identical to SEQ ID NO: 485; (1) nucleotide 12 through nucleotide 36 of a sequence that is at least 90% identical to SEQ ID NO: 485; (m) nucleotide 13 through nucleotide 36 of a sequence that is at least 90% identical to SEQ ID NO: 485; (n) nucleotide 14 through nucleotide 36 of a sequence that is at least 90% identical to SEQ ID NO: 485; or (o) a sequence that is at least 90% identical to a sequence of SEQ ID NO: 486 or SEQ ID NO: 487 or a portion thereof.

In some examples, the direct repeat sequence comprises: (a) nucleotide 1 through nucleotide 36 of SEQ ID NO: 485; (b) nucleotide 2 through nucleotide 36 of SEQ ID NO: 485; (c) nucleotide 3 through nucleotide 36 of SEQ ID NO: 485; (d) nucleotide 4 through nucleotide 36 of SEQ ID NO: 485; (c) nucleotide 5 through nucleotide 36 of SEQ ID NO: 485; (f) nucleotide 6 through nucleotide 36 of SEQ ID NO: 485; (g) nucleotide 7 through nucleotide 36 of SEQ ID NO: 485; (h) nucleotide 8 through nucleotide 36 of SEQ ID NO: 485; (i) nucleotide 9 through nucleotide 36 of SEQ ID NO: 485; (j) nucleotide 10 through nucleotide 36 of SEQ ID NO: 485; (k) nucleotide 11 through nucleotide 36 of SEQ ID NO: 485; (1) nucleotide 12 through nucleotide 36 of SEQ ID NO: 485; (m) nucleotide 13 through nucleotide 36 of SEQ ID NO: 485; (n) nucleotide 14 through nucleotide 36 of SEQ ID NO: 485; or (0) SEQ ID NO: 486 or SEQ ID NO: 487 or a portion thereof.

In some examples, the direct repeat sequence comprises: (a) nucleotide 1 through nucleotide 36 of a sequence that is at least 90% identical to a sequence of SEQ ID NO: 488 or SEQ ID NO: 489; (b) nucleotide 2 through nucleotide 36 of a sequence that is at least 90% identical to a sequence of SEQ ID NO: 488 or SEQ ID NO: 489; (c) nucleotide 3 through nucleotide 36 of a sequence that is at least 90% identical to a sequence of SEQ ID NO: 488 or SEQ ID NO: 489; (d) nucleotide 4 through nucleotide 36 of a sequence that is at least 90% identical to a sequence of SEQ ID NO: 488 or SEQ ID NO: 489; (c) nucleotide 5 through nucleotide 36 of a sequence that is at least 90% identical to a sequence of SEQ ID NO: 488 or SEQ ID NO: 489; (f) nucleotide 6 through nucleotide 36 of a sequence that is at least 90% identical to a sequence of SEQ ID NO: 488 or SEQ ID NO: 489; (g) nucleotide 7 through nucleotide 36 of a sequence that is at least 90% identical to a sequence of SEQ ID NO: 488 or SEQ ID NO: 489; (h) nucleotide 8 through nucleotide 36 of a sequence that is at least 90% identical to a sequence of SEQ ID NO: 488 or SEQ ID NO: 489; (i) nucleotide 9 through nucleotide 36 of a sequence that is at least 90% identical to a sequence of SEQ ID NO: 488 or SEQ ID NO: 489; (j) nucleotide 10 through nucleotide 36 of a sequence that is at least 90% identical to a sequence of SEQ ID NO: 488 or SEQ ID NO: 489; (k) nucleotide 11 through nucleotide 36 of a sequence that is at least 90% identical to a sequence of SEQ ID NO: 488 or SEQ ID NO: 489; (1) nucleotide 12 through nucleotide 36 of a sequence that is at least 90% identical to a sequence of SEQ ID NO: 488 or SEQ ID NO: 489; (m) nucleotide 13 through nucleotide 36 of a sequence that is at least 90% identical to a sequence of SEQ ID NO: 488 or SEQ ID NO: 489; (n) nucleotide 14 through nucleotide 36 of a sequence that is at least 90% identical to a sequence of SEQ ID NO: 488 or SEQ ID NO: 489; (0) nucleotide 15 through nucleotide 36 of a sequence that is at least 90% identical to a sequence of SEQ ID NO: 488 or SEQ ID NO: 489; or (p) a sequence that is at least 90% identical to a sequence of SEQ ID NO: 490 or a portion thereof.

In some examples, the direct repeat sequence comprises: (a) nucleotide 1 through nucleotide 36 of SEQ ID NO: 488 or SEQ ID NO: 489; (b) nucleotide 2 through nucleotide 36 of SEQ ID NO: 488 or SEQ ID NO: 489; (c) nucleotide 3 through nucleotide 36 of SEQ ID NO: 488 or SEQ ID NO: 489; (d) nucleotide 4 through nucleotide 36 of SEQ ID NO: 488 or SEQ ID NO: 489; (c) nucleotide 5 through nucleotide 36 of SEQ ID NO: 488 or SEQ ID NO: 489; (f) nucleotide 6 through nucleotide 36 of SEQ ID NO: 488 or SEQ ID NO: 489; (g) nucleotide 7 through nucleotide 36 of SEQ ID NO: 488 or SEQ ID NO: 489; (h) nucleotide 8 through nucleotide 36 of SEQ ID NO: 488 or SEQ ID NO: 489; (i) nucleotide 9 through nucleotide 36 of SEQ ID NO: 488 or SEQ ID NO: 489; (j) nucleotide 10 through nucleotide 36 of SEQ ID NO: 488 or SEQ ID NO: 489; (k) nucleotide 11 through nucleotide 36 of SEQ ID NO: 488 or SEQ ID NO: 489; (1) nucleotide 12 through nucleotide 36 of SEQ ID NO: 488 or SEQ ID NO: 489; (m) nucleotide 13 through nucleotide 36 of SEQ ID NO: 488 or SEQ ID NO: 489; (n) nucleotide 14 through nucleotide 36 of SEQ ID NO: 488 or SEQ ID NO: 489; (0) nucleotide 15 through nucleotide 36 of SEQ ID NO: 488 or SEQ ID NO: 489; or (p) SEQ ID NO: 490 or a portion thereof.

In some examples, the spacer sequence is substantially complementary to the complement of a sequence of any one of SEQ ID NOs: 11-228 or 491-2496.

In any of the composition of Embodiment 1, the PAM may comprise the sequence 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

In some examples, the target sequence is immediately adjacent to the PAM sequence.

In some examples, the RNA guide has a sequence that is at least 90% identical to a sequence of any one of SEQ ID NOs: 4505-4562.

In some examples, the RNA guide has the sequence of any one of SEQ ID NOs: 4505-4562.

Embodiment 2: The composition of Embodiment 1 may further comprise a Cas12i polypeptide or a polyribonucleotide encoding a Cas12i polypeptide, which can be one of the following: (a) a Cas12i2 polypeptide comprising a sequence that is at least 90% identical to the sequence of SEQ ID NO: 448, SEQ ID NO: 449, SEQ ID NO: 450, SEQ ID NO: 451, SEQ ID NO: 452, or SEQ ID NO: 453; (b) a Cas12i4 polypeptide comprising a sequence that is at least 90% identical to the sequence of SEQ ID NO: 482, SEQ ID NO: 483, or SEQ ID NO: 484; (c) a Cas12i1 polypeptide comprising a sequence that is at least 90% identical to the sequence of SEQ ID NO: 4503; or (d) a Cas12i3 polypeptide comprising a sequence that is at least 90% identical to the sequence of SEQ ID NO: 4504.

In specific examples, the Cas12i polypeptide is: (a) a Cas12i2 polypeptide comprising a sequence of SEQ ID NO: 448, SEQ ID NO: 449, SEQ ID NO: 450, SEQ ID NO: 451. SEQ ID NO: 452, or SEQ ID NO: 453; (b) a Cas12i4 polypeptide comprising a sequence of SEQ ID NO: 482, SEQ ID NO: 483, or SEQ ID NO: 484; (c) a Cas12i1 polypeptide comprising a sequence of SEQ ID NO: 4503; or (d) a Cas12i3 polypeptide comprising a sequence of SEQ ID NO: 4504.

In any of the compositions of Embodiment 2, the RNA guide and the Cas12i polypeptide may form a ribonucleoprotein complex. In some examples, the ribonucleoprotein complex binds a target nucleic acid. In some examples, the composition is present within a cell.

In any of the compositions of Embodiment 2, the RNA guide and the Cas12i polypeptide may be encoded in a vector, e.g., expression vector. In some examples, the RNA guide and the Cas12i polypeptide are encoded in a single vector. In other examples, the RNA guide is encoded in a first vector and the Cas12i polypeptide is encoded in a second vector.

Embodiment 3: A vector system comprising one or more vectors encoding an RNA guide disclosed herein and a Cas12i polypeptide. In some examples, the vector system comprises a first vector encoding an RNA guide disclosed herein and a second vector encoding a Cas12i polypeptide. The vectors may be expression vectors.

Embodiment 4: A composition comprising an RNA guide and a Cas12i polypeptide, wherein the RNA guide comprises (i) a spacer sequence that is substantially complementary or completely complementary to a region on a non-PAM strand (the complementary sequence of a target sequence) within an STMN2 gene, and (ii) a direct repeat sequence.

In some examples, the target sequence is within exon 1, exon 2, exon 3, exon 4, exon 5, exon 6, exon 7, or an intron of the STMN2 gene, which may comprise the sequence of SEQ ID NO: 454, the reverse complement of SEQ ID NO: 454, a variant of the sequence of SEQ ID NO: 454, or the reverse complement of a variant of SEQ ID NO: 454.

In some examples, the spacer sequence comprises: (a) nucleotide 1 through nucleotide 16 of a sequence that is at least 90% identical to a sequence of any one of SEQ ID NOs: 229-446 or 2497-4502; (b) nucleotide 1 through nucleotide 17 of a sequence that is at least 90% identical to a sequence of any one of SEQ ID NOs: 229-446 or 2497-4502; (c) nucleotide 1 through nucleotide 18 of a sequence that is at least 90% identical to a sequence of any one of SEQ ID NOs: 229-446 or 2497-4502; (d) nucleotide 1 through nucleotide 19 of a sequence that is at least 90% identical to a sequence of any one of SEQ ID NOs: 229-446 or 2497-4502; (c) nucleotide 1 through nucleotide 20 of a sequence that is at least 90% identical to a sequence of any one of SEQ ID NOs: 229-446 or 2497-4502; (f) nucleotide 1 through nucleotide 21 of a sequence that is at least 90% identical to a sequence of any one of SEQ ID NOs: 229-446 or 2497-4502; (g) nucleotide 1 through nucleotide 22 of a sequence that is at least 90% identical to a sequence of any one of SEQ ID NOs: 229-446 or 2497-4502; (h) nucleotide 1 through nucleotide 23 of a sequence that is at least 90% identical to a sequence of any one of SEQ ID NOs: 229-446 or 2497-4502; (i) nucleotide 1 through nucleotide 24 of a sequence that is at least 90% identical to a sequence of any one of SEQ ID NOs: 229-446 or 2497-4502; (j) nucleotide 1 through nucleotide 25 of a sequence that is at least 90% identical to a sequence of any one of SEQ ID NOs: 229-446 or 2497-4502; (k) nucleotide 1 through nucleotide 26 of a sequence that is at least 90% identical to a sequence of any one of SEQ ID NOs: 229-446 or 2497-4502; (1) nucleotide 1 through nucleotide 27 of a sequence that is at least 90% identical to a sequence of any one of SEQ ID NOs: 229-446 or 2497-4502; (m) nucleotide 1 through nucleotide 28 of a sequence that is at least 90% identical to a sequence of any one of SEQ ID NOs: 229-446 or 2497-4502; (n) nucleotide 1 through nucleotide 29 of a sequence that is at least 90% identical to a sequence of any one of SEQ ID NOs: 229-446 or 2497-4502; or (o) nucleotide 1 through nucleotide 30 of a sequence that is at least 90% identical to a sequence of any one of SEQ ID NOs: 229-446 or 2497-4502.

In some examples, the spacer sequence comprises: (a) nucleotide 1 through nucleotide 16 of any one of SEQ ID NOs: 229-446 or 2497-4502; (b) nucleotide 1 through nucleotide 17 of any one of SEQ ID NOs: 229-446 or 2497-4502; (c) nucleotide 1 through nucleotide 18 of any one of SEQ ID NOs: 229-446 or 2497-4502; (d) nucleotide 1 through nucleotide 19 of any one of SEQ ID NOs: 229-446 or 2497-4502; (c) nucleotide 1 through nucleotide 20 of any one of SEQ ID NOs: 229-446 or 2497-4502; (f) nucleotide 1 through nucleotide 21 of any one of SEQ ID NOs: 229-446 or 2497-4502; (g) nucleotide 1 through nucleotide 22 of any one of SEQ ID NOs: 229-446 or 2497-4502; (h) nucleotide 1 through nucleotide 23 of any one of SEQ ID NOs: 229-446 or 2497-4502; (i) nucleotide 1 through nucleotide 24 of any one of SEQ ID NOs: 229-446 or 2497-4502; (j) nucleotide 1 through nucleotide 25 of any one of SEQ ID NOs: 229-446 or 2497-4502; (k) nucleotide 1 through nucleotide 26 of any one of SEQ ID NOs: 229-446 or 2497-4502; (1) nucleotide 1 through nucleotide 27 of any one of SEQ ID NOs: 229-446 or 2497-4502; (m) nucleotide 1 through nucleotide 28 of any one of SEQ ID NOs: 229-446 or 2497-4502; (n) nucleotide 1 through nucleotide 29 of any one of SEQ ID NOs: 229-446 or 2497-4502; or (0) nucleotide 1 through nucleotide 30 of any one of SEQ ID NOs: 229-446 or 2497-4502.

In some examples, the direct repeat sequence comprises: (a) nucleotide 1 through nucleotide 36 of a sequence that is at least 90% identical to a sequence of any one of SEQ ID NOs: 1-8; (b) nucleotide 2 through nucleotide 36 of a sequence that is at least 90% identical to a sequence of any one of SEQ ID NOs: 1-8; (c) nucleotide 3 through nucleotide 36 of a sequence that is at least 90% identical to a sequence of any one of SEQ ID NOs: 1-8; (d) nucleotide 4 through nucleotide 36 of a sequence that is at least 90% identical to a sequence of any one of SEQ ID NOs: 1-8; (c) nucleotide 5 through nucleotide 36 of a sequence that is at least 90% identical to a sequence of any one of SEQ ID NOs: 1-8; (f) nucleotide 6 through nucleotide 36 of a sequence that is at least 90% identical to a sequence of any one of SEQ ID NOs: 1-8; (g) nucleotide 7 through nucleotide 36 of a sequence that is at least 90% identical to a sequence of any one of SEQ ID NOs: 1-8; (h) nucleotide 8 through nucleotide 36 of a sequence that is at least 90% identical to a sequence of any one of SEQ ID NOs: 1-8; (i) nucleotide 9 through nucleotide 36 of a sequence that is at least 90% identical to a sequence of any one of SEQ ID NOs: 1-8; (j) nucleotide 10 through nucleotide 36 of a sequence that is at least 90% identical to a sequence of any one of SEQ ID NOs: 1-8; (k) nucleotide 11 through nucleotide 36 of a sequence that is at least 90% identical to a sequence of any one of SEQ ID NOs: 1-8; (1) nucleotide 12 through nucleotide 36 of a sequence that is at least 90% identical to a sequence of any one of SEQ ID NOs: 1-8; (m) nucleotide 13 through nucleotide 36 of a sequence that is at least 90% identical to a sequence of any one of SEQ ID NOs: 1-8; (n) nucleotide 14 through nucleotide 36 of a sequence that is at least 90% identical to a sequence of any one of SEQ ID NOs: 1-8; (0) nucleotide 1 through nucleotide 34 of a sequence that is at least 90% identical to a sequence of SEQ ID NO: 9; (p) nucleotide 2 through nucleotide 34 of a sequence that is at least 90% identical to a sequence of SEQ ID NO: 9; (q) nucleotide 3 through nucleotide 34 of a sequence that is at least 90% identical to a sequence of SEQ ID NO: 9; (r) nucleotide 4 through nucleotide 34 of a sequence that is at least 90% identical to a sequence of SEQ ID NO: 9; (s) nucleotide 5 through nucleotide 34 of a sequence that is at least 90% identical to a sequence of SEQ ID NO: 9; (t) nucleotide 6 through nucleotide 34 of a sequence that is at least 90% identical to a sequence of SEQ ID NO: 9; (u) nucleotide 7 through nucleotide 34 of a sequence that is at least 90% identical to a sequence of SEQ ID NO: 9; (v) nucleotide 8 through nucleotide 34 of a sequence that is at least 90% identical to a sequence of SEQ ID NO: 9; (w) nucleotide 9 through nucleotide 34 of a sequence that is at least 90% identical to a sequence of SEQ ID NO: 9; (x) nucleotide 10 through nucleotide 34 of a sequence that is at least 90% identical to a sequence of SEQ ID NO: 9; (y) nucleotide 11 through nucleotide 34 of a sequence that is at least 90% identical to a sequence of SEQ ID NO: 9; (2) nucleotide 12 through nucleotide 34 of a sequence that is at least 90% identical to a sequence of SEQ ID NO: 9; or (aa) a sequence that is at least 90% identical to a sequence of SEQ ID NO: 10 or a portion thereof.

In some examples, the direct repeat sequence comprises: (a) nucleotide 1 through nucleotide 36 of any one of SEQ ID NOs: 1-8; (b) nucleotide 2 through nucleotide 36 of any one of SEQ ID NOs: 1-8; (c) nucleotide 3 through nucleotide 36 of any one of SEQ ID NOs: 1-8; (d) nucleotide 4 through nucleotide 36 of any one of SEQ ID NOs: 1-8; (c) nucleotide 5 through nucleotide 36 of any one of SEQ ID NOs: 1-8; (f) nucleotide 6 through nucleotide 36 of any one of SEQ ID NOs: 1-8; (g) nucleotide 7 through nucleotide 36 of any one of SEQ ID NOs: 1-8; (h) nucleotide 8 through nucleotide 36 of any one of SEQ ID NOs: 1-8; (i) nucleotide 9 through nucleotide 36 of any one of SEQ ID NOs: 1-8; (j) nucleotide 10 through nucleotide 36 of any one of SEQ ID NOs: 1-8; (k) nucleotide 11 through nucleotide 36 of any one of SEQ ID NOs: 1-8; (1) nucleotide 12 through nucleotide 36 of any one of SEQ ID NOs: 1-8; (m) nucleotide 13 through nucleotide 36 of any one of SEQ ID NOs: 1-8; (n) nucleotide 14 through nucleotide 36 of any one of SEQ ID NOs: 1-8; (o) nucleotide 1 through nucleotide 34 of SEQ ID NO: 9; (p) nucleotide 2 through nucleotide 34 of SEQ ID NO: 9; (q) nucleotide 3 through nucleotide 34 of SEQ ID NO: 9; (r) nucleotide 4 through nucleotide 34 of SEQ ID NO: 9; (s) nucleotide 5 through nucleotide 34 of SEQ ID NO: 9; (t) nucleotide 6 through nucleotide 34 of SEQ ID NO: 9; (u) nucleotide 7 through nucleotide 34 of SEQ ID NO: 9; (v) nucleotide 8 through nucleotide 34 of SEQ ID NO: 9; (w) nucleotide 9 through nucleotide 34 of SEQ ID NO: 9; (x) nucleotide 10 through nucleotide 34 of SEQ ID NO: 9; (y) nucleotide 11 through nucleotide 34 of SEQ ID NO: 9; (z) nucleotide 12 through nucleotide 34 of SEQ ID NO: 9; or (aa) SEQ ID NO: 10 or a portion thereof.

In some examples, the direct repeat sequence comprises: (a) nucleotide 1 through nucleotide 36 of a sequence that is at least 90% identical to a sequence of any one of SEQ ID NOs: 462-479; (b) nucleotide 2 through nucleotide 36 of a sequence that is at least 90% identical to a sequence of any one of SEQ ID NOs: 462-479; (c) nucleotide 3 through nucleotide 36 of a sequence that is at least 90% identical to a sequence of any one of SEQ ID NOs: 462-479; (d) nucleotide 4 through nucleotide 36 of a sequence that is at least 90% identical to a sequence of any one of SEQ ID NOs: 462-479; (c) nucleotide 5 through nucleotide 36 of a sequence that is at least 90% identical to a sequence of any one of SEQ ID NOs: 462-479; (f) nucleotide 6 through nucleotide 36 of a sequence that is at least 90% identical to a sequence of any one of SEQ ID NOs: 462-479; (g) nucleotide 7 through nucleotide 36 of a sequence that is at least 90% identical to a sequence of any one of SEQ ID NOs: 462-479; (h) nucleotide 8 through nucleotide 36 of a sequence that is at least 90% identical to a sequence of any one of SEQ ID NOs: 462-479; (i) nucleotide 9 through nucleotide 36 of a sequence that is at least 90% identical to a sequence of any one of SEQ ID NOs: 462-479; (j) nucleotide 10 through nucleotide 36 of a sequence that is at least 90% identical to a sequence of any one of SEQ ID NOs: 462-479; (k) nucleotide 11 through nucleotide 36 of a sequence that is at least 90% identical to a sequence of any one of SEQ ID NOs: 462-479; (1) nucleotide 12 through nucleotide 36 of a sequence that is at least 90% identical to a sequence of any one of SEQ ID NOs: 462-479; (m) nucleotide 13 through nucleotide 36 of a sequence that is at least 90% identical to a sequence of any one of SEQ ID NOs: 462-479; (n) nucleotide 14 through nucleotide 36 of a sequence that is at least 90% identical to a sequence of any one of SEQ ID NOs: 462-479; or (o) a sequence that is at least 90% identical to a sequence of SEQ ID NO: 480 or a portion thereof.

In some examples, the direct repeat sequence comprises: (a) nucleotide 1 through nucleotide 36 of any one of SEQ ID NOs: 462-479; (b) nucleotide 2 through nucleotide 36 of any one of SEQ ID NOs: 462-479; (c) nucleotide 3 through nucleotide 36 of any one of SEQ ID NOs: 462-479; (d) nucleotide 4 through nucleotide 36 of any one of SEQ ID NOs: 462-479; (c) nucleotide 5 through nucleotide 36 of any one of SEQ ID NOs: 462-479; (f) nucleotide 6 through nucleotide 36 of any one of SEQ ID NOs: 462-479; (g) nucleotide 7 through nucleotide 36 of any one of SEQ ID NOs: 462-479; (h) nucleotide 8 through nucleotide 36 of any one of SEQ ID NOs: 462-479; (i) nucleotide 9 through nucleotide 36 of any one of SEQ ID NOs: 462-479; (j) nucleotide 10 through nucleotide 36 of any one of SEQ ID NOs: 462-479; (k) nucleotide 11 through nucleotide 36 of any one of SEQ ID NOs: 462-479; (1) nucleotide 12 through nucleotide 36 of any one of SEQ ID NOs: 462-479; (m) nucleotide 13 through nucleotide 36 of any one of SEQ ID NOs: 462-479; (n) nucleotide 14 through nucleotide 36 of any one of SEQ ID NOs: 462-479; or (0) SEQ ID NO: 480 or a portion thereof.

In some examples, the direct repeat sequence comprises: (a) nucleotide 1 through nucleotide 36 of a sequence that is at least 90% identical to SEQ ID NO: 485; (b) nucleotide 2 through nucleotide 36 of a sequence that is at least 90% identical to SEQ ID NO: 485; (c) nucleotide 3 through nucleotide 36 of a sequence that is at least 90% identical to SEQ ID NO: 485; (d) nucleotide 4 through nucleotide 36 of a sequence that is at least 90% identical to SEQ ID NO: 485; (c) nucleotide 5 through nucleotide 36 of a sequence that is at least 90% identical to SEQ ID NO: 485; (f) nucleotide 6 through nucleotide 36 of a sequence that is at least 90% identical to SEQ ID NO: 485; (g) nucleotide 7 through nucleotide 36 of a sequence that is at least 90% identical to SEQ ID NO: 485; (h) nucleotide 8 through nucleotide 36 of a sequence that is at least 90% identical to SEQ ID NO: 485; (i) nucleotide 9 through nucleotide 36 of a sequence that is at least 90% identical to SEQ ID NO: 485; (j) nucleotide 10 through nucleotide 36 of a sequence that is at least 90% identical to SEQ ID NO: 485; (k) nucleotide 11 through nucleotide 36 of a sequence that is at least 90% identical to SEQ ID NO: 485; (1) nucleotide 12 through nucleotide 36 of a sequence that is at least 90% identical to SEQ ID NO: 485; (m) nucleotide 13 through nucleotide 36 of a sequence that is at least 90% identical to SEQ ID NO: 485; (n) nucleotide 14 through nucleotide 36 of a sequence that is at least 90% identical to SEQ ID NO: 485; or (o) a sequence that is at least 90% identical to a sequence of SEQ ID NO: 486 or SEQ ID NO: 487 or a portion thereof.

In some examples, the direct repeat sequence comprises: (a) nucleotide 1 through nucleotide 36 of SEQ ID NO: 485; (b) nucleotide 2 through nucleotide 36 of SEQ ID NO: 485; (c) nucleotide 3 through nucleotide 36 of SEQ ID NO: 485; (d) nucleotide 4 through nucleotide 36 of SEQ ID NO: 485; (c) nucleotide 5 through nucleotide 36 of SEQ ID NO: 485; (f) nucleotide 6 through nucleotide 36 of SEQ ID NO: 485; (g) nucleotide 7 through nucleotide 36 of SEQ ID NO: 485; (h) nucleotide 8 through nucleotide 36 of SEQ ID NO: 485; (i) nucleotide 9 through nucleotide 36 of SEQ ID NO: 485; (j) nucleotide 10 through nucleotide 36 of SEQ ID NO: 485; (k) nucleotide 11 through nucleotide 36 of SEQ ID NO: 485; (1) nucleotide 12 through nucleotide 36 of SEQ ID NO: 485; (m) nucleotide 13 through nucleotide 36 of SEQ ID NO: 485; (n) nucleotide 14 through nucleotide 36 of SEQ ID NO: 485; or (0) SEQ ID NO: 486 or SEQ ID NO: 487 or a portion thereof.

In some examples, the direct repeat sequence comprises: (a) nucleotide 1 through nucleotide 36 of a sequence that is at least 90% identical to a sequence of SEQ ID NO: 488 or SEQ ID NO: 489; (b) nucleotide 2 through nucleotide 36 of a sequence that is at least 90% identical to a sequence of SEQ ID NO: 488 or SEQ ID NO: 489; (c) nucleotide 3 through nucleotide 36 of a sequence that is at least 90% identical to a sequence of SEQ ID NO: 488 or SEQ ID NO: 489; (d) nucleotide 4 through nucleotide 36 of a sequence that is at least 90% identical to a sequence of SEQ ID NO: 488 or SEQ ID NO: 489; (c) nucleotide 5 through nucleotide 36 of a sequence that is at least 90% identical to a sequence of SEQ ID NO: 488 or SEQ ID NO: 489; (f) nucleotide 6 through nucleotide 36 of a sequence that is at least 90% identical to a sequence of SEQ ID NO: 488 or SEQ ID NO: 489; (g) nucleotide 7 through nucleotide 36 of a sequence that is at least 90% identical to a sequence of SEQ ID NO: 488 or SEQ ID NO: 489; (h) nucleotide 8 through nucleotide 36 of a sequence that is at least 90% identical to a sequence of SEQ ID NO: 488 or SEQ ID NO: 489; (i) nucleotide 9 through nucleotide 36 of a sequence that is at least 90% identical to a sequence of SEQ ID NO: 488 or SEQ ID NO: 489; (j) nucleotide 10 through nucleotide 36 of a sequence that is at least 90% identical to a sequence of SEQ ID NO: 488 or SEQ ID NO: 489; (k) nucleotide 11 through nucleotide 36 of a sequence that is at least 90% identical to a sequence of SEQ ID NO: 488 or SEQ ID NO: 489; (1) nucleotide 12 through nucleotide 36 of a sequence that is at least 90% identical to a sequence of SEQ ID NO: 488 or SEQ ID NO: 489; (m) nucleotide 13 through nucleotide 36 of a sequence that is at least 90% identical to a sequence of SEQ ID NO: 488 or SEQ ID NO: 489; (n) nucleotide 14 through nucleotide 36 of a sequence that is at least 90% identical to a sequence of SEQ ID NO: 488 or SEQ ID NO: 489; (o) nucleotide 15 through nucleotide 36 of a sequence that is at least 90% identical to a sequence of SEQ ID NO: 488 or SEQ ID NO: 489; or (p) a sequence that is at least 90% identical to a sequence of SEQ ID NO: 490 or a portion thereof.

In some examples, the direct repeat sequence comprises: (a) nucleotide 1 through nucleotide 36 of SEQ ID NO: 488 or SEQ ID NO: 489; (b) nucleotide 2 through nucleotide 36 of SEQ ID NO: 488 or SEQ ID NO: 489; (c) nucleotide 3 through nucleotide 36 of SEQ ID NO: 488 or SEQ ID NO: 489; (d) nucleotide 4 through nucleotide 36 of SEQ ID NO: 488 or SEQ ID NO: 489; (c) nucleotide 5 through nucleotide 36 of SEQ ID NO: 488 or SEQ ID NO: 489; (f) nucleotide 6 through nucleotide 36 of SEQ ID NO: 488 or SEQ ID NO: 489; (g) nucleotide 7 through nucleotide 36 of SEQ ID NO: 488 or SEQ ID NO: 489; (h) nucleotide 8 through nucleotide 36 of SEQ ID NO: 488 or SEQ ID NO: 489; (i) nucleotide 9 through nucleotide 36 of SEQ ID NO: 488 or SEQ ID NO: 489; (j) nucleotide 10 through nucleotide 36 of SEQ ID NO: 488 or SEQ ID NO: 489; (k) nucleotide 11 through nucleotide 36 of SEQ ID NO: 488 or SEQ ID NO: 489; (l) nucleotide 12 through nucleotide 36 of SEQ ID NO: 488 or SEQ ID NO: 489; (m) nucleotide 13 through nucleotide 36 of SEQ ID NO: 488 or SEQ ID NO: 489; (n) nucleotide 14 through nucleotide 36 of SEQ ID NO: 488 or SEQ ID NO: 489; (o) nucleotide 15 through nucleotide 36 of SEQ ID NO: 488 or SEQ ID NO: 489; or (p) SEQ ID NO: 490 or a portion thereof.

In any of the compositions of Embodiment 4, the spacer sequence may be substantially complementary to the complement of a sequence of any one of SEQ ID NOs: 11-228 or 491-2496.

In some examples, the target sequence is adjacent to a protospacer adjacent motif (PAM) comprising the sequence 5′-NTTN-3′. In some examples, the PAM comprises the sequence 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

In some examples, the target sequence is immediately adjacent to the PAM sequence. In some examples, the target sequence is within 1, 2, 3, 4, or 5 nucleotides of the PAM sequence.

In any of the compositions of Embodiment 4, the Cas12i polypeptide is: (a) a Cas12i2 polypeptide comprising a sequence that is at least 90% identical to the sequence of SEQ ID NO: 448, SEQ ID NO: 449, SEQ ID NO: 450, SEQ ID NO: 451, SEQ ID NO: 452, or SEQ ID NO: 453; (b) a Cas12i4 polypeptide comprising a sequence that is at least 90% identical to the sequence of SEQ ID NO: 482, SEQ ID NO: 483, or SEQ ID NO: 484; (c) a Cas12i1 polypeptide comprising a sequence that is at least 90% identical to the sequence of SEQ ID NO: 4503; or (d) a Cas12i3 polypeptide comprising a sequence that is at least 90% identical to the sequence of SEQ ID NO: 4504.

In some examples, the Cas12i polypeptide is: (a) a Cas12i2 polypeptide comprising a sequence of SEQ ID NO: 448, SEQ ID NO: 449, SEQ ID NO: 450, SEQ ID NO: 451, SEQ ID NO: 452, or SEQ ID NO: 453; (b) a Cas12i4 polypeptide comprising a sequence of SEQ ID NO: 482, SEQ ID NO: 483, or SEQ ID NO: 484; (c) a Cas12i1 polypeptide comprising a sequence of SEQ ID NO: 4503; or (d) a Cas1213 polypeptide comprising a sequence of SEQ ID NO: 4504.

In any of the composition of Embodiment 4, the RNA guide and the Cas12i polypeptide may form a ribonucleoprotein complex. In some examples, the ribonucleoprotein complex binds a target nucleic acid.

In any of the composition of Embodiment 4, the composition may be present within a cell.

In any of the composition of Embodiment 4, the RNA guide and the Cas12i polypeptide may be encoded in a vector, e.g., expression vector. In some examples, the RNA guide and the Cas12i polypeptide are encoded in a single vector. In other examples, the RNA guide is encoded in a first vector and the Cas12i polypeptide is encoded in a second vector.

Embodiment 5: A vector system comprising one or more vectors encoding an RNA guide disclosed herein and a Cas12i polypeptide. In some examples, the vector system comprises a first vector encoding an RNA guide disclosed herein and a second vector encoding a Cas12i polypeptide. In some examples, the vectors are expression vectors.

Embodiment 6: An RNA guide comprising (i) a spacer sequence that is substantially complementary or completely complementary to a region on a non-PAM strand (the complementary sequence of a target sequence) within an STMN2 gene, and (ii) a direct repeat sequence.

In some examples, the target sequence is within exon 1, exon 2, exon 3, exon 4, exon 5, exon 6, exon 7, or an intron of the STMN2 gene, which may comprise the sequence of SEQ ID NO: 454, the reverse complement of SEQ ID NO: 454, a variant of the sequence of SEQ ID NO: 454, or the reverse complement of a variant of SEQ ID NO: 454.

In some examples, the spacer sequence comprises: (a) nucleotide 1 through nucleotide 16 of a sequence that is at least 90% identical to a sequence of any one of SEQ ID NOs: 229-446 or 2497-4502; (b) nucleotide 1 through nucleotide 17 of a sequence that is at least 90% identical to a sequence of any one of SEQ ID NOs: 229-446 or 2497-4502; (c) nucleotide 1 through nucleotide 18 of a sequence that is at least 90% identical to a sequence of any one of SEQ ID NOs: 229-446 or 2497-4502; (d) nucleotide 1 through nucleotide 19 of a sequence that is at least 90% identical to a sequence of any one of SEQ ID NOs: 229-446 or 2497-4502; (c) nucleotide 1 through nucleotide 20 of a sequence that is at least 90% identical to a sequence of any one of SEQ ID NOs: 229-446 or 2497-4502; (f) nucleotide 1 through nucleotide 21 of a sequence that is at least 90% identical to a sequence of any one of SEQ ID NOs: 229-446 or 2497-4502; (g) nucleotide 1 through nucleotide 22 of a sequence that is at least 90% identical to a sequence of any one of SEQ ID NOs: 229-446 or 2497-4502; (h) nucleotide 1 through nucleotide 23 of a sequence that is at least 90% identical to a sequence of any one of SEQ ID NOs: 229-446 or 2497-4502; (i) nucleotide 1 through nucleotide 24 of a sequence that is at least 90% identical to a sequence of any one of SEQ ID NOs: 229-446 or 2497-4502; (j) nucleotide 1 through nucleotide 25 of a sequence that is at least 90% identical to a sequence of any one of SEQ ID NOs: 229-446 or 2497-4502; (k) nucleotide 1 through nucleotide 26 of a sequence that is at least 90% identical to a sequence of any one of SEQ ID NOs: 229-446 or 2497-4502; (1) nucleotide 1 through nucleotide 27 of a sequence that is at least 90% identical to a sequence of any one of SEQ ID NOs: 229-446 or 2497-4502; (m) nucleotide 1 through nucleotide 28 of a sequence that is at least 90% identical to a sequence of any one of SEQ ID NOs: 229-446 or 2497-4502; (n) nucleotide 1 through nucleotide 29 of a sequence that is at least 90% identical to a sequence of any one of SEQ ID NOs: 229-446 or 2497-4502; or (o) nucleotide 1 through nucleotide 30 of a sequence that is at least 90% identical to a sequence of any one of SEQ ID NOs: 229-446 or 2497-4502.

In some examples, the spacer sequence comprises: (a) nucleotide 1 through nucleotide 16 of any one of SEQ ID NOs: 229-446 or 2497-4502; (b) nucleotide 1 through nucleotide 17 of any one of SEQ ID NOs: 229-446 or 2497-4502; (c) nucleotide 1 through nucleotide 18 of any one of SEQ ID NOs: 229-446 or 2497-4502; (d) nucleotide 1 through nucleotide 19 of any one of SEQ ID NOs: 229-446 or 2497-4502; (c) nucleotide 1 through nucleotide 20 of any one of SEQ ID NOs: 229-446 or 2497-4502; (f) nucleotide 1 through nucleotide 21 of any one of SEQ ID NOs: 229-446 or 2497-4502; (g) nucleotide 1 through nucleotide 22 of any one of SEQ ID NOs: 229-446 or 2497-4502; (h) nucleotide 1 through nucleotide 23 of any one of SEQ ID NOs: 229-446 or 2497-4502; (i) nucleotide 1 through nucleotide 24 of any one of SEQ ID NOs: 229-446 or 2497-4502; (j) nucleotide 1 through nucleotide 25 of any one of SEQ ID NOs: 229-446 or 2497-4502; (k) nucleotide 1 through nucleotide 26 of any one of SEQ ID NOs: 229-446 or 2497-4502; (l) nucleotide 1 through nucleotide 27 of any one of SEQ ID NOs: 229-446 or 2497-4502; (m) nucleotide 1 through nucleotide 28 of any one of SEQ ID NOs: 229-446 or 2497-4502; (n) nucleotide 1 through nucleotide 29 of any one of SEQ ID NOs: 229-446 or 2497-4502; or (0) nucleotide 1 through nucleotide 30 of any one of SEQ ID NOs: 229-446 or 2497-4502.

In some examples, the direct repeat sequence comprises: (a) nucleotide 1 through nucleotide 36 of a sequence that is at least 90% identical to a sequence of any one of SEQ ID NOs: 1-8; (b) nucleotide 2 through nucleotide 36 of a sequence that is at least 90% identical to a sequence of any one of SEQ ID NOs: 1-8; (c) nucleotide 3 through nucleotide 36 of a sequence that is at least 90% identical to a sequence of any one of SEQ ID NOs: 1-8; (d) nucleotide 4 through nucleotide 36 of a sequence that is at least 90% identical to a sequence of any one of SEQ ID NOs: 1-8; (e) nucleotide 5 through nucleotide 36 of a sequence that is at least 90% identical to a sequence of any one of SEQ ID NOs: 1-8; (f) nucleotide 6 through nucleotide 36 of a sequence that is at least 90% identical to a sequence of any one of SEQ ID NOs: 1-8; (g) nucleotide 7 through nucleotide 36 of a sequence that is at least 90% identical to a sequence of any one of SEQ ID NOs: 1-8; (h) nucleotide 8 through nucleotide 36 of a sequence that is at least 90% identical to a sequence of any one of SEQ ID NOs: 1-8; (i) nucleotide 9 through nucleotide 36 of a sequence that is at least 90% identical to a sequence of any one of SEQ ID NOs: 1-8; (j) nucleotide 10 through nucleotide 36 of a sequence that is at least 90% identical to a sequence of any one of SEQ ID NOs: 1-8; (k) nucleotide 11 through nucleotide 36 of a sequence that is at least 90% identical to a sequence of any one of SEQ ID NOs: 1-8; (1) nucleotide 12 through nucleotide 36 of a sequence that is at least 90% identical to a sequence of any one of SEQ ID NOs: 1-8; (m) nucleotide 13 through nucleotide 36 of a sequence that is at least 90% identical to a sequence of any one of SEQ ID NOs: 1-8; (n) nucleotide 14 through nucleotide 36 of a sequence that is at least 90% identical to a sequence of any one of SEQ ID NOs: 1-8; (0) nucleotide 1 through nucleotide 34 of a sequence that is at least 90% identical to a sequence of SEQ ID NO: 9; (p) nucleotide 2 through nucleotide 34 of a sequence that is at least 90% identical to a sequence of SEQ ID NO: 9; (q) nucleotide 3 through nucleotide 34 of a sequence that is at least 90% identical to a sequence of SEQ ID NO: 9; (r) nucleotide 4 through nucleotide 34 of a sequence that is at least 90% identical to a sequence of SEQ ID NO: 9; (s) nucleotide 5 through nucleotide 34 of a sequence that is at least 90% identical to a sequence of SEQ ID NO: 9; (t) nucleotide 6 through nucleotide 34 of a sequence that is at least 90% identical to a sequence of SEQ ID NO: 9; (u) nucleotide 7 through nucleotide 34 of a sequence that is at least 90% identical to a sequence of SEQ ID NO: 9; (v) nucleotide 8 through nucleotide 34 of a sequence that is at least 90% identical to a sequence of SEQ ID NO: 9; (w) nucleotide 9 through nucleotide 34 of a sequence that is at least 90% identical to a sequence of SEQ ID NO: 9; (x) nucleotide 10 through nucleotide 34 of a sequence that is at least 90% identical to a sequence of SEQ ID NO: 9; (y) nucleotide 11 through nucleotide 34 of a sequence that is at least 90% identical to a sequence of SEQ ID NO: 9; (2) nucleotide 12 through nucleotide 34 of a sequence that is at least 90% identical to a sequence of SEQ ID NO: 9; or (aa) a sequence that is at least 90% identical to a sequence of SEQ ID NO: 10 or a portion thereof.

In some examples, the direct repeat sequence comprises: (a) nucleotide 1 through nucleotide 36 of any one of SEQ ID NOs: 1-8; (b) nucleotide 2 through nucleotide 36 of any one of SEQ ID NOs: 1-8; (c) nucleotide 3 through nucleotide 36 of any one of SEQ ID NOs: 1-8; (d) nucleotide 4 through nucleotide 36 of any one of SEQ ID NOs: 1-8; (c) nucleotide 5 through nucleotide 36 of any one of SEQ ID NOs: 1-8; (f) nucleotide 6 through nucleotide 36 of any one of SEQ ID NOs: 1-8; (g) nucleotide 7 through nucleotide 36 of any one of SEQ ID NOs: 1-8; (h) nucleotide 8 through nucleotide 36 of any one of SEQ ID NOs: 1-8; (i) nucleotide 9 through nucleotide 36 of any one of SEQ ID NOs: 1-8; (j) nucleotide 10 through nucleotide 36 of any one of SEQ ID NOs: 1-8; (k) nucleotide 11 through nucleotide 36 of any one of SEQ ID NOs: 1-8; (1) nucleotide 12 through nucleotide 36 of any one of SEQ ID NOs: 1-8; (m) nucleotide 13 through nucleotide 36 of any one of SEQ ID NOs: 1-8; (n) nucleotide 14 through nucleotide 36 of any one of SEQ ID NOs: 1-8; (o) nucleotide 1 through nucleotide 34 of SEQ ID NO: 9; (p) nucleotide 2 through nucleotide 34 of SEQ ID NO: 9; (q) nucleotide 3 through nucleotide 34 of SEQ ID NO: 9; (r) nucleotide 4 through nucleotide 34 of SEQ ID NO: 9; (s) nucleotide 5 through nucleotide 34 of SEQ ID NO: 9; (t) nucleotide 6 through nucleotide 34 of SEQ ID NO: 9; (u) nucleotide 7 through nucleotide 34 of SEQ ID NO: 9; (v) nucleotide 8 through nucleotide 34 of SEQ ID NO: 9; (w) nucleotide 9 through nucleotide 34 of SEQ ID NO: 9; (x) nucleotide 10 through nucleotide 34 of SEQ ID NO: 9; (y) nucleotide 11 through nucleotide 34 of SEQ ID NO: 9; (z) nucleotide 12 through nucleotide 34 of SEQ ID NO: 9; or (aa) SEQ ID NO: 10 or a portion thereof.

In some examples, the direct repeat sequence comprises: (a) nucleotide 1 through nucleotide 36 of a sequence that is at least 90% identical to a sequence of any one of SEQ ID NOs: 462-479; (b) nucleotide 2 through nucleotide 36 of a sequence that is at least 90% identical to a sequence of any one of SEQ ID NOs: 462-479; (c) nucleotide 3 through nucleotide 36 of a sequence that is at least 90% identical to a sequence of any one of SEQ ID NOs: 462-479; (d) nucleotide 4 through nucleotide 36 of a sequence that is at least 90% identical to a sequence of any one of SEQ ID NOs: 462-479; (c) nucleotide 5 through nucleotide 36 of a sequence that is at least 90% identical to a sequence of any one of SEQ ID NOs: 462-479; (f) nucleotide 6 through nucleotide 36 of a sequence that is at least 90% identical to a sequence of any one of SEQ ID NOs: 462-479; (g) nucleotide 7 through nucleotide 36 of a sequence that is at least 90% identical to a sequence of any one of SEQ ID NOs: 462-479; (h) nucleotide 8 through nucleotide 36 of a sequence that is at least 90% identical to a sequence of any one of SEQ ID NOs: 462-479; (i) nucleotide 9 through nucleotide 36 of a sequence that is at least 90% identical to a sequence of any one of SEQ ID NOs: 462-479; (j) nucleotide 10 through nucleotide 36 of a sequence that is at least 90% identical to a sequence of any one of SEQ ID NOs: 462-479; (k) nucleotide 11 through nucleotide 36 of a sequence that is at least 90% identical to a sequence of any one of SEQ ID NOs: 462-479; (1) nucleotide 12 through nucleotide 36 of a sequence that is at least 90% identical to a sequence of any one of SEQ ID NOs: 462-479; (m) nucleotide 13 through nucleotide 36 of a sequence that is at least 90% identical to a sequence of any one of SEQ ID NOs: 462-479; (n) nucleotide 14 through nucleotide 36 of a sequence that is at least 90% identical to a sequence of any one of SEQ ID NOs: 462-479; or (o) a sequence that is at least 90% identical to a sequence of SEQ ID NO: 480 or a portion thereof.

In some examples, the direct repeat sequence comprises: (a) nucleotide 1 through nucleotide 36 of any one of SEQ ID NOs: 462-479; (b) nucleotide 2 through nucleotide 36 of any one of SEQ ID NOs: 462-479; (c) nucleotide 3 through nucleotide 36 of any one of SEQ ID NOs: 462-479; (d) nucleotide 4 through nucleotide 36 of any one of SEQ ID NOs: 462-479; (c) nucleotide 5 through nucleotide 36 of any one of SEQ ID NOs: 462-479; (f) nucleotide 6 through nucleotide 36 of any one of SEQ ID NOs: 462-479; (g) nucleotide 7 through nucleotide 36 of any one of SEQ ID NOs: 462-479; (h) nucleotide 8 through nucleotide 36 of any one of SEQ ID NOs: 462-479; (i) nucleotide 9 through nucleotide 36 of any one of SEQ ID NOs: 462-479; (j) nucleotide 10 through nucleotide 36 of any one of SEQ ID NOs: 462-479; (k) nucleotide 11 through nucleotide 36 of any one of SEQ ID NOs: 462-479; (1) nucleotide 12 through nucleotide 36 of any one of SEQ ID NOs: 462-479; (m) nucleotide 13 through nucleotide 36 of any one of SEQ ID NOs: 462-479; (n) nucleotide 14 through nucleotide 36 of any one of SEQ ID NOs: 462-479; or (o) SEQ ID NO: 480 or a portion thereof.

In some examples, the direct repeat sequence comprises: (a) nucleotide 1 through nucleotide 36 of a sequence that is at least 90% identical to SEQ ID NO: 485; (b) nucleotide 2 through nucleotide 36 of a sequence that is at least 90% identical to SEQ ID NO: 485; (c) nucleotide 3 through nucleotide 36 of a sequence that is at least 90% identical to SEQ ID NO: 485; (d) nucleotide 4 through nucleotide 36 of a sequence that is at least 90% identical to SEQ ID NO: 485; (c) nucleotide 5 through nucleotide 36 of a sequence that is at least 90% identical to SEQ ID NO: 485; (f) nucleotide 6 through nucleotide 36 of a sequence that is at least 90% identical to SEQ ID NO: 485; (g) nucleotide 7 through nucleotide 36 of a sequence that is at least 90% identical to SEQ ID NO: 485; (h) nucleotide 8 through nucleotide 36 of a sequence that is at least 90% identical to SEQ ID NO: 485; (i) nucleotide 9 through nucleotide 36 of a sequence that is at least 90% identical to SEQ ID NO: 485; (j) nucleotide 10 through nucleotide 36 of a sequence that is at least 90% identical to SEQ ID NO: 485; (k) nucleotide 11 through nucleotide 36 of a sequence that is at least 90% identical to SEQ ID NO: 485; (1) nucleotide 12 through nucleotide 36 of a sequence that is at least 90% identical to SEQ ID NO: 485; (m) nucleotide 13 through nucleotide 36 of a sequence that is at least 90% identical to SEQ ID NO: 485; (n) nucleotide 14 through nucleotide 36 of a sequence that is at least 90% identical to SEQ ID NO: 485; or (o) a sequence that is at least 90% identical to a sequence of SEQ ID NO: 486 or SEQ ID NO: 487 or a portion thereof.

In some examples, the direct repeat sequence comprises: (a) nucleotide 1 through nucleotide 36 of SEQ ID NO: 485; (b) nucleotide 2 through nucleotide 36 of SEQ ID NO: 485; (c) nucleotide 3 through nucleotide 36 of SEQ ID NO: 485; (d) nucleotide 4 through nucleotide 36 of SEQ ID NO: 485; (c) nucleotide 5 through nucleotide 36 of SEQ ID NO: 485; (f) nucleotide 6 through nucleotide 36 of SEQ ID NO: 485; (g) nucleotide 7 through nucleotide 36 of SEQ ID NO: 485; (h) nucleotide 8 through nucleotide 36 of SEQ ID NO: 485; (i) nucleotide 9 through nucleotide 36 of SEQ ID NO: 485; (j) nucleotide 10 through nucleotide 36 of SEQ ID NO: 485; (k) nucleotide 11 through nucleotide 36 of SEQ ID NO: 485; (1) nucleotide 12 through nucleotide 36 of SEQ ID NO: 485; (m) nucleotide 13 through nucleotide 36 of SEQ ID NO: 485; (n) nucleotide 14 through nucleotide 36 of SEQ ID NO: 485; or (0) SEQ ID NO: 486 or SEQ ID NO: 487 or a portion thereof.

In some examples, the direct repeat sequence comprises: (a) nucleotide 1 through nucleotide 36 of a sequence that is at least 90% identical to a sequence of SEQ ID NO: 488 or SEQ ID NO: 489; (b) nucleotide 2 through nucleotide 36 of a sequence that is at least 90% identical to a sequence of SEQ ID NO: 488 or SEQ ID NO: 489; (c) nucleotide 3 through nucleotide 36 of a sequence that is at least 90% identical to a sequence of SEQ ID NO: 488 or SEQ ID NO: 489; (d) nucleotide 4 through nucleotide 36 of a sequence that is at least 90% identical to a sequence of SEQ ID NO: 488 or SEQ ID NO: 489; (c) nucleotide 5 through nucleotide 36 of a sequence that is at least 90% identical to a sequence of SEQ ID NO: 488 or SEQ ID NO: 489; (f) nucleotide 6 through nucleotide 36 of a sequence that is at least 90% identical to a sequence of SEQ ID NO: 488 or SEQ ID NO: 489; (g) nucleotide 7 through nucleotide 36 of a sequence that is at least 90% identical to a sequence of SEQ ID NO: 488 or SEQ ID NO: 489; (h) nucleotide 8 through nucleotide 36 of a sequence that is at least 90% identical to a sequence of SEQ ID NO: 488 or SEQ ID NO: 489; (i) nucleotide 9 through nucleotide 36 of a sequence that is at least 90% identical to a sequence of SEQ ID NO: 488 or SEQ ID NO: 489; (j) nucleotide 10 through nucleotide 36 of a sequence that is at least 90% identical to a sequence of SEQ ID NO: 488 or SEQ ID NO: 489; (k) nucleotide 11 through nucleotide 36 of a sequence that is at least 90% identical to a sequence of SEQ ID NO: 488 or SEQ ID NO: 489; (1) nucleotide 12 through nucleotide 36 of a sequence that is at least 90% identical to a sequence of SEQ ID NO: 488 or SEQ ID NO: 489; (m) nucleotide 13 through nucleotide 36 of a sequence that is at least 90% identical to a sequence of SEQ ID NO: 488 or SEQ ID NO: 489; (n) nucleotide 14 through nucleotide 36 of a sequence that is at least 90% identical to a sequence of SEQ ID NO: 488 or SEQ ID NO: 489; (o) nucleotide 15 through nucleotide 36 of a sequence that is at least 90% identical to a sequence of SEQ ID NO: 488 or SEQ ID NO: 489; or (p) a sequence that is at least 90% identical to a sequence of SEQ ID NO: 490 or a portion thereof.

In some examples, the direct repeat sequence comprises: (a) nucleotide 1 through nucleotide 36 of SEQ ID NO: 488 or SEQ ID NO: 489; (b) nucleotide 2 through nucleotide 36 of SEQ ID NO: 488 or SEQ ID NO: 489; (c) nucleotide 3 through nucleotide 36 of SEQ ID NO: 488 or SEQ ID NO: 489; (d) nucleotide 4 through nucleotide 36 of SEQ ID NO: 488 or SEQ ID NO: 489; (c) nucleotide 5 through nucleotide 36 of SEQ ID NO: 488 or SEQ ID NO: 489; (f) nucleotide 6 through nucleotide 36 of SEQ ID NO: 488 or SEQ ID NO: 489; (g) nucleotide 7 through nucleotide 36 of SEQ ID NO: 488 or SEQ ID NO: 489; (h) nucleotide 8 through nucleotide 36 of SEQ ID NO: 488 or SEQ ID NO: 489; (i) nucleotide 9 through nucleotide 36 of SEQ ID NO: 488 or SEQ ID NO: 489; (j) nucleotide 10 through nucleotide 36 of SEQ ID NO: 488 or SEQ ID NO: 489; (k) nucleotide 11 through nucleotide 36 of SEQ ID NO: 488 or SEQ ID NO: 489; (1) nucleotide 12 through nucleotide 36 of SEQ ID NO: 488 or SEQ ID NO: 489; (m) nucleotide 13 through nucleotide 36 of SEQ ID NO: 488 or SEQ ID NO: 489; (n) nucleotide 14 through nucleotide 36 of SEQ ID NO: 488 or SEQ ID NO: 489; (o) nucleotide 15 through nucleotide 36 of SEQ ID NO: 488 or SEQ ID NO: 489; or (p) SEQ ID NO: 490 or a portion thereof.

In any of the RNA guide of Embodiment 6, the spacer sequence may be substantially complementary to the complement of a sequence of any one of SEQ ID NOs: 11-228 or 491-2496.

In any of the RNA guide of Embodiment 6, the target sequence may be adjacent to a protospacer adjacent motif (PAM) comprising the sequence 5′-NTTN-3′, wherein N is any nucleotide. In some examples, the PAM comprises the sequence 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

In some examples, the target sequence is immediately adjacent to the PAM sequence. In other examples, the target sequence is within 1, 2, 3, 4, or 5 nucleotides of the PAM sequence.

In some examples, the RNA guide has a sequence that is at least 90% identical to a sequence of any one of SEQ ID NOs: 4505-4562. In specific examples, the RNA guide has the sequence of any one of SEQ ID NOs: 4505-4562.

Embodiment 7: A nucleic acid encoding an RNA guide as described herein.

Embodiment 8: A vector comprising such an RNA guide as described herein.

Embodiment 9: A cell comprising a composition, an RNA guide, a nucleic acid, or a vector as described herein. In some examples, the cell is a eukaryotic cell, an animal cell, a mammalian cell, a human cell, a primary cell, a cell line, a stem cell, a neuron, or a T cell.

Embodiment 10: A kit comprising a composition, an RNA guide, a nucleic acid, or a vector as described herein.

Embodiment 11: A method of editing an STMN2 sequence, the method comprising contacting an STMN2 sequence with a composition or an RNA guide as described herein. In some examples, the method is carried out in vitro. In other examples, the method is carried out ex vivo.

In some examples, the STMN2 sequence is in a cell.

In some examples, the composition or the RNA guide induces a deletion in the STMN2 sequence. In some examples, the deletion is adjacent to a 5′-NTTN-3′ sequence, wherein N is any nucleotide. In some specific examples, the deletion is downstream of the 5′-NTTN-3′ sequence. In some specific examples, the deletion is up to about 40 nucleotides in length. In some instances, the deletion is from about 4 nucleotides to 40 nucleotides, about 4 nucleotides to 25 nucleotides, about 10 nucleotides to 25 nucleotides, or about 10 nucleotides to 15 nucleotides in length.

In some examples, the deletion starts within about 5 nucleotides to about 15 nucleotides, about 5 nucleotides to about 10 nucleotides, or about 10 nucleotides to about 15 nucleotides of the 5′-NTTN-3′ sequence.

In some examples, the deletion starts within about 5 nucleotides to about 15 nucleotides, about 5 nucleotides to about 10 nucleotides, or about 10 nucleotides to about 15 nucleotides downstream of the 5′-NTTN-3′ sequence.

In some examples, the deletion ends within about 20 nucleotides to about 30 nucleotides, about 20 nucleotides to about 25 nucleotides, or about 25 nucleotides to about 30 nucleotides of the 5′-NTTN-3′ sequence.

In some examples, the deletion ends within about 20 nucleotides to about 30 nucleotides, about 20 nucleotides to about 25 nucleotides, about 25 nucleotides to about 30 nucleotides downstream of the 5′-NTTN-3′ sequence.

In some examples, the deletion starts within about 5 nucleotides to about 15 nucleotides downstream of the 5′-NTTN-3′ sequence and ends within about 20 nucleotides to about 30 nucleotides downstream of the 5′-NTTN-3′ sequence.

In some examples, the deletion starts within about 5 nucleotides to about 15 nucleotides downstream of the 5′-NTTN-3′ sequence and ends within about 20 nucleotides to about 25 nucleotides downstream of the 5′-NTTN-3′ sequence.

In some examples, the deletion starts within about 5 nucleotides to about 15 nucleotides downstream of the 5′-NTTN-3′ sequence and ends within about 25 nucleotides to about 30 nucleotides downstream of the 5′-NTTN-3′ sequence.

In some examples, the deletion starts within about 5 nucleotides to about 10 nucleotides downstream of the 5′-NTTN-3′ sequence and ends within about 20 nucleotides to about 30 nucleotides downstream of the 5′-NTTN-3′ sequence.

In some examples, the deletion starts within about 5 nucleotides to about 10 nucleotides downstream of the 5′-NTTN-3′ sequence and ends within about 20 nucleotides to about 25 nucleotides downstream of the 5′-NTTN-3′ sequence.

In some examples, the deletion starts within about 5 nucleotides to about 10 nucleotides downstream of the 5′-NTTN-3′ sequence and ends within about 25 nucleotides to about 30 nucleotides downstream of the 5′-NTTN-3′ sequence.

In some examples, the deletion starts within about 10 nucleotides to about 15 nucleotides downstream of the 5′-NTTN-3′ sequence and ends within about 20 nucleotides to about 30 nucleotides downstream of the 5′-NTTN-3′ sequence.

In some examples, the deletion starts within about 10 nucleotides to about 15 nucleotides downstream of the 5′-NTTN-3′ sequence and ends within about 20 nucleotides to about 25 nucleotides downstream of the 5′-NTTN-3′ sequence.

In some examples, the deletion starts within about 10 nucleotides to about 15 nucleotides downstream of the 5′-NTTN-3′ sequence and ends within about 25 nucleotides to about 30 nucleotides downstream of the 5′-NTTN-3′ sequence.

In some examples, the 5′-NTTN-3′ sequence is 5′-CTTT-3′, 5′-CTTC-3′, 5′-GTTT-3′, 5′-GTTC-3′, 5′-TTTC-3′, 5′-GTTA-3′, or 5′-GTTG-3′.

In some examples, the deletion overlaps with a mutation in the STMN2 sequence. In some instances, the deletion overlaps with an insertion in the STMN2 sequence. In some instances, the deletion removes a repeat expansion of the STMN2 sequence or a portion thereof. In some instances, the deletion disrupts one or both alleles of the STMN2 sequence.

In any of the composition, RNA guide, nucleic acid, vector, cell, kit, or method of Embodiments 1-11 described herein, the RNA guide may comprise the sequence of any one of SEQ ID NOs: 4505-4562.

Embodiment 12: A method of treating neurodegenerative diseases (e.g., amyotrophic lateral sclerosis (ALS) or frontotemporal dementia (FTD)) in a subject, the method comprising administering a composition, an RNA guide, or a cell described herein to the subject.

In any of the compositions, RNA guides, cells, kits, or methods described herein, the RNA guide and/or the polyribonucleotide encoding the Cas12i polypeptide are comprised within a lipid nanoparticle. In some examples, the RNA guide and the polyribonucleotide encoding the Cas12i polypeptide are comprised within the same lipid nanoparticle. In other examples, the RNA guide and the polyribonucleotide encoding the Cas12i polypeptide are comprised within separate lipid nanoparticles.

Embodiment 13: An RNA guide comprising (i) a spacer sequence that is complementary to a target site within an STMN2 gene (the target site being on the non-PAM strand and complementary to a target sequence), and (ii) a direct repeat sequence.

In some examples, the direct repeat sequence comprises: (a) nucleotide 1 through nucleotide 36 of a sequence that is at least 90% identical to a sequence of any one of SEQ ID NOs: 1-8; (b) nucleotide 2 through nucleotide 36 of a sequence that is at least 90% identical to a sequence of any one of SEQ ID NOs: 1-8; (c) nucleotide 3 through nucleotide 36 of a sequence that is at least 90% identical to a sequence of any one of SEQ ID NOs: 1-8; (d) nucleotide 4 through nucleotide 36 of a sequence that is at least 90% identical to a sequence of any one of SEQ ID NOs: 1-8; (e) nucleotide 5 through nucleotide 36 of a sequence that is at least 90% identical to a sequence of any one of SEQ ID NOs: 1-8; (f) nucleotide 6 through nucleotide 36 of a sequence that is at least 90% identical to a sequence of any one of SEQ ID NOs: 1-8; (g) nucleotide 7 through nucleotide 36 of a sequence that is at least 90% identical to a sequence of any one of SEQ ID NOs: 1-8; (h) nucleotide 8 through nucleotide 36 of a sequence that is at least 90% identical to a sequence of any one of SEQ ID NOs: 1-8; (i) nucleotide 9 through nucleotide 36 of a sequence that is at least 90% identical to a sequence of any one of SEQ ID NOs: 1-8; (j) nucleotide 10 through nucleotide 36 of a sequence that is at least 90% identical to a sequence of any one of SEQ ID NOs: 1-8; (k) nucleotide 11 through nucleotide 36 of a sequence that is at least 90% identical to a sequence of any one of SEQ ID NOs: 1-8; (1) nucleotide 12 through nucleotide 36 of a sequence that is at least 90% identical to a sequence of any one of SEQ ID NOs: 1-8; (m) nucleotide 13 through nucleotide 36 of a sequence that is at least 90% identical to a sequence of any one of SEQ ID NOs: 1-8; (n) nucleotide 14 through nucleotide 36 of a sequence that is at least 90% identical to a sequence of any one of SEQ ID NOs: 1-8; (o) nucleotide 1 through nucleotide 34 of a sequence that is at least 90% identical to a sequence of SEQ ID NO: 9; (p) nucleotide 2 through nucleotide 34 of a sequence that is at least 90% identical to a sequence of SEQ ID NO: 9; (q) nucleotide 3 through nucleotide 34 of a sequence that is at least 90% identical to a sequence of SEQ ID NO: 9; (r) nucleotide 4 through nucleotide 34 of a sequence that is at least 90% identical to a sequence of SEQ ID NO: 9; (s) nucleotide 5 through nucleotide 34 of a sequence that is at least 90% identical to a sequence of SEQ ID NO: 9; (t) nucleotide 6 through nucleotide 34 of a sequence that is at least 90% identical to a sequence of SEQ ID NO: 9; (u) nucleotide 7 through nucleotide 34 of a sequence that is at least 90% identical to a sequence of SEQ ID NO: 9; (v) nucleotide 8 through nucleotide 34 of a sequence that is at least 90% identical to a sequence of SEQ ID NO: 9; (w) nucleotide 9 through nucleotide 34 of a sequence that is at least 90% identical to a sequence of SEQ ID NO: 9; (x) nucleotide 10 through nucleotide 34 of a sequence that is at least 90% identical to a sequence of SEQ ID NO: 9; (y) nucleotide 11 through nucleotide 34 of a sequence that is at least 90% identical to a sequence of SEQ ID NO: 9; (z) nucleotide 12 through nucleotide 34 of a sequence that is at least 90% identical to a sequence of SEQ ID NO: 9; or (aa) a sequence that is at least 90% identical to a sequence of SEQ ID NO: 10 or a portion thereof.

In some examples, the direct repeat sequence comprises: (a) nucleotide 1 through nucleotide 36 of any one of SEQ ID NOs: 1-8; (b) nucleotide 2 through nucleotide 36 of any one of SEQ ID NOs: 1-8; (c) nucleotide 3 through nucleotide 36 of any one of SEQ ID NOs: 1-8; (d) nucleotide 4 through nucleotide 36 of any one of SEQ ID NOs: 1-8; (c) nucleotide 5 through nucleotide 36 of any one of SEQ ID NOs: 1-8; (f) nucleotide 6 through nucleotide 36 of any one of SEQ ID NOs: 1-8; (g) nucleotide 7 through nucleotide 36 of any one of SEQ ID NOs: 1-8; (h) nucleotide 8 through nucleotide 36 of any one of SEQ ID NOs: 1-8; (i) nucleotide 9 through nucleotide 36 of any one of SEQ ID NOs: 1-8; (j) nucleotide 10 through nucleotide 36 of any one of SEQ ID NOs: 1-8; (k) nucleotide 11 through nucleotide 36 of any one of SEQ ID NOs: 1-8; (1) nucleotide 12 through nucleotide 36 of any one of SEQ ID NOs: 1-8; (m) nucleotide 13 through nucleotide 36 of any one of SEQ ID NOs: 1-8; (n) nucleotide 14 through nucleotide 36 of any one of SEQ ID NOs: 1-8; (o) nucleotide 1 through nucleotide 34 of SEQ ID NO: 9; (p) nucleotide 2 through nucleotide 34 of SEQ ID NO: 9; (q) nucleotide 3 through nucleotide 34 of SEQ ID NO: 9; (r) nucleotide 4 through nucleotide 34 of SEQ ID NO: 9; (s) nucleotide 5 through nucleotide 34 of SEQ ID NO: 9; (t) nucleotide 6 through nucleotide 34 of SEQ ID NO: 9; (u) nucleotide 7 through nucleotide 34 of SEQ ID NO: 9; (v) nucleotide 8 through nucleotide 34 of SEQ ID NO: 9; (w) nucleotide 9 through nucleotide 34 of SEQ ID NO: 9; (x) nucleotide 10 through nucleotide 34 of SEQ ID NO: 9; (y) nucleotide 11 through nucleotide 34 of SEQ ID NO: 9; (z) nucleotide 12 through nucleotide 34 of SEQ ID NO: 9; or (aa) SEQ ID NO: 10 or a portion thereof.

In some examples, the direct repeat sequence comprises: (a) nucleotide 1 through nucleotide 36 of a sequence that is at least 90% identical to a sequence of any one of SEQ ID NOs: 462-479; (b) nucleotide 2 through nucleotide 36 of a sequence that is at least 90% identical to a sequence of any one of SEQ ID NOs: 462-479; (c) nucleotide 3 through nucleotide 36 of a sequence that is at least 90% identical to a sequence of any one of SEQ ID NOs: 462-479; (d) nucleotide 4 through nucleotide 36 of a sequence that is at least 90% identical to a sequence of any one of SEQ ID NOs: 462-479; (e) nucleotide 5 through nucleotide 36 of a sequence that is at least 90% identical to a sequence of any one of SEQ ID NOs: 462-479; (f) nucleotide 6 through nucleotide 36 of a sequence that is at least 90% identical to a sequence of any one of SEQ ID NOs: 462-479; (g) nucleotide 7 through nucleotide 36 of a sequence that is at least 90% identical to a sequence of any one of SEQ ID NOs: 462-479; (h) nucleotide 8 through nucleotide 36 of a sequence that is at least 90% identical to a sequence of any one of SEQ ID NOs: 462-479; (i) nucleotide 9 through nucleotide 36 of a sequence that is at least 90% identical to a sequence of any one of SEQ ID NOs: 462-479; (j) nucleotide 10 through nucleotide 36 of a sequence that is at least 90% identical to a sequence of any one of SEQ ID NOs: 462-479; (k) nucleotide 11 through nucleotide 36 of a sequence that is at least 90% identical to a sequence of any one of SEQ ID NOs: 462-479; (1) nucleotide 12 through nucleotide 36 of a sequence that is at least 90% identical to a sequence of any one of SEQ ID NOs: 462-479; (m) nucleotide 13 through nucleotide 36 of a sequence that is at least 90% identical to a sequence of any one of SEQ ID NOs: 462-479; (n) nucleotide 14 through nucleotide 36 of a sequence that is at least 90% identical to a sequence of any one of SEQ ID NOs: 462-479; or (o) a sequence that is at least 90% identical to a sequence of SEQ ID NO: 480 or a portion thereof.

In some examples, the direct repeat sequence comprises: (a) nucleotide 1 through nucleotide 36 of any one of SEQ ID NOs: 462-479; (b) nucleotide 2 through nucleotide 36 of any one of SEQ ID NOs: 462-479; (c) nucleotide 3 through nucleotide 36 of any one of SEQ ID NOs: 462-479; (d) nucleotide 4 through nucleotide 36 of any one of SEQ ID NOs: 462-479; (c) nucleotide 5 through nucleotide 36 of any one of SEQ ID NOs: 462-479; (f) nucleotide 6 through nucleotide 36 of any one of SEQ ID NOs: 462-479; (g) nucleotide 7 through nucleotide 36 of any one of SEQ ID NOs: 462-479; (h) nucleotide 8 through nucleotide 36 of any one of SEQ ID NOs: 462-479; (i) nucleotide 9 through nucleotide 36 of any one of SEQ ID NOs: 462-479; (j) nucleotide 10 through nucleotide 36 of any one of SEQ ID NOs: 462-479; (k) nucleotide 11 through nucleotide 36 of any one of SEQ ID NOs: 462-479; (1) nucleotide 12 through nucleotide 36 of any one of SEQ ID NOs: 462-479; (m) nucleotide 13 through nucleotide 36 of any one of SEQ ID NOs: 462-479; (n) nucleotide 14 through nucleotide 36 of any one of SEQ ID NOs: 462-479; or (o) SEQ ID NO: 480 or a portion thereof.

In some examples, the direct repeat sequence comprises: (a) nucleotide 1 through nucleotide 36 of a sequence that is at least 90% identical to SEQ ID NO: 485; (b) nucleotide 2 through nucleotide 36 of a sequence that is at least 90% identical to SEQ ID NO: 485; (c) nucleotide 3 through nucleotide 36 of a sequence that is at least 90% identical to SEQ ID NO: 485; (d) nucleotide 4 through nucleotide 36 of a sequence that is at least 90% identical to SEQ ID NO: 485; (c) nucleotide 5 through nucleotide 36 of a sequence that is at least 90% identical to SEQ ID NO: 485; (f) nucleotide 6 through nucleotide 36 of a sequence that is at least 90% identical to SEQ ID NO: 485; (g) nucleotide 7 through nucleotide 36 of a sequence that is at least 90% identical to SEQ ID NO: 485; (h) nucleotide 8 through nucleotide 36 of a sequence that is at least 90% identical to SEQ ID NO: 485; (i) nucleotide 9 through nucleotide 36 of a sequence that is at least 90% identical to SEQ ID NO: 485; (j) nucleotide 10 through nucleotide 36 of a sequence that is at least 90% identical to SEQ ID NO: 485; (k) nucleotide 11 through nucleotide 36 of a sequence that is at least 90% identical to SEQ ID NO: 485; (1) nucleotide 12 through nucleotide 36 of a sequence that is at least 90% identical to SEQ ID NO: 485; (m) nucleotide 13 through nucleotide 36 of a sequence that is at least 90% identical to SEQ ID NO: 485; (n) nucleotide 14 through nucleotide 36 of a sequence that is at least 90% identical to SEQ ID NO: 485; or (o) a sequence that is at least 90% identical to a sequence of SEQ ID NO: 486 or SEQ ID NO: 487 or a portion thereof.

In some examples, the direct repeat sequence comprises: (a) nucleotide 1 through nucleotide 36 of SEQ ID NO: 485; (b) nucleotide 2 through nucleotide 36 of SEQ ID NO: 485; (c) nucleotide 3 through nucleotide 36 of SEQ ID NO: 485; (d) nucleotide 4 through nucleotide 36 of SEQ ID NO: 485; (c) nucleotide 5 through nucleotide 36 of SEQ ID NO: 485; (f) nucleotide 6 through nucleotide 36 of SEQ ID NO: 485; (g) nucleotide 7 through nucleotide 36 of SEQ ID NO: 485; (h) nucleotide 8 through nucleotide 36 of SEQ ID NO: 485; (i) nucleotide 9 through nucleotide 36 of SEQ ID NO: 485; (j) nucleotide 10 through nucleotide 36 of SEQ ID NO: 485; (k) nucleotide 11 through nucleotide 36 of SEQ ID NO: 485; (1) nucleotide 12 through nucleotide 36 of SEQ ID NO: 485; (m) nucleotide 13 through nucleotide 36 of SEQ ID NO: 485; (n) nucleotide 14 through nucleotide 36 of SEQ ID NO: 485; or (0) SEQ ID NO: 486 or SEQ ID NO: 487 or a portion thereof.

In some examples, the direct repeat sequence comprises: (a) nucleotide 1 through nucleotide 36 of a sequence that is at least 90% identical to a sequence of SEQ ID NO: 488 or SEQ ID NO: 489; (b) nucleotide 2 through nucleotide 36 of a sequence that is at least 90% identical to a sequence of SEQ ID NO: 488 or SEQ ID NO: 489; (c) nucleotide 3 through nucleotide 36 of a sequence that is at least 90% identical to a sequence of SEQ ID NO: 488 or SEQ ID NO: 489; (d) nucleotide 4 through nucleotide 36 of a sequence that is at least 90% identical to a sequence of SEQ ID NO: 488 or SEQ ID NO: 489; (e) nucleotide 5 through nucleotide 36 of a sequence that is at least 90% identical to a sequence of SEQ ID NO: 488 or SEQ ID NO: 489; (f) nucleotide 6 through nucleotide 36 of a sequence that is at least 90% identical to a sequence of SEQ ID NO: 488 or SEQ ID NO: 489; (g) nucleotide 7 through nucleotide 36 of a sequence that is at least 90% identical to a sequence of SEQ ID NO: 488 or SEQ ID NO: 489; (h) nucleotide 8 through nucleotide 36 of a sequence that is at least 90% identical to a sequence of SEQ ID NO: 488 or SEQ ID NO: 489; (i) nucleotide 9 through nucleotide 36 of a sequence that is at least 90% identical to a sequence of SEQ ID NO: 488 or SEQ ID NO: 489; (j) nucleotide 10 through nucleotide 36 of a sequence that is at least 90% identical to a sequence of SEQ ID NO: 488 or SEQ ID NO: 489; (k) nucleotide 11 through nucleotide 36 of a sequence that is at least 90% identical to a sequence of SEQ ID NO: 488 or SEQ ID NO: 489; (1) nucleotide 12 through nucleotide 36 of a sequence that is at least 90% identical to a sequence of SEQ ID NO: 488 or SEQ ID NO: 489; (m) nucleotide 13 through nucleotide 36 of a sequence that is at least 90% identical to a sequence of SEQ ID NO: 488 or SEQ ID NO: 489; (n) nucleotide 14 through nucleotide 36 of a sequence that is at least 90% identical to a sequence of SEQ ID NO: 488 or SEQ ID NO: 489; (o) nucleotide 15 through nucleotide 36 of a sequence that is at least 90% identical to a sequence of SEQ ID NO: 488 or SEQ ID NO: 489; or (p) a sequence that is at least 90% identical to a sequence of SEQ ID NO: 490 or a portion thereof.

In some examples, the direct repeat sequence comprises: (a) nucleotide 1 through nucleotide 36 of SEQ ID NO: 488 or SEQ ID NO: 489; (b) nucleotide 2 through nucleotide 36 of SEQ ID NO: 488 or SEQ ID NO: 489; (c) nucleotide 3 through nucleotide 36 of SEQ ID NO: 488 or SEQ ID NO: 489; (d) nucleotide 4 through nucleotide 36 of SEQ ID NO: 488 or SEQ ID NO: 489; (c) nucleotide 5 through nucleotide 36 of SEQ ID NO: 488 or SEQ ID NO: 489; (f) nucleotide 6 through nucleotide 36 of SEQ ID NO: 488 or SEQ ID NO: 489; (g) nucleotide 7 through nucleotide 36 of SEQ ID NO: 488 or SEQ ID NO: 489; (h) nucleotide 8 through nucleotide 36 of SEQ ID NO: 488 or SEQ ID NO: 489; (i) nucleotide 9 through nucleotide 36 of SEQ ID NO: 488 or SEQ ID NO: 489; (j) nucleotide 10 through nucleotide 36 of SEQ ID NO: 488 or SEQ ID NO: 489; (k) nucleotide 11 through nucleotide 36 of SEQ ID NO: 488 or SEQ ID NO: 489; (1) nucleotide 12 through nucleotide 36 of SEQ ID NO: 488 or SEQ ID NO: 489; (m) nucleotide 13 through nucleotide 36 of SEQ ID NO: 488 or SEQ ID NO: 489; (n) nucleotide 14 through nucleotide 36 of SEQ ID NO: 488 or SEQ ID NO: 489; (o) nucleotide 15 through nucleotide 36 of SEQ ID NO: 488 or SEQ ID NO: 489; or (p) SEQ ID NO: 490 or a portion thereof.

In some examples, each of the first three nucleotides of the RNA guide comprises a 2′-O-methyl phosphorothioate modification.

In some examples, each of the last four nucleotides of the RNA guide comprises a 2′-O-methyl phosphorothioate modification.

In some examples, each of the first to last, second to last, and third to last nucleotides of the RNA guide comprises a 2′-O-methyl phosphorothioate modification, and wherein the last nucleotide of the RNA guide is unmodified.

Embodiment 14: A nucleic acid encoding an RNA guide as described herein.

Embodiment 15: A vector comprising the nucleic acid as described herein.

Embodiment 16: A vector system comprising one or more vectors encoding (i) the RNA guide of Embodiment 13 as described herein and (ii) a Cas12i polypeptide. In some examples, the vector system comprises a first vector encoding the RNA guide and a second vector encoding the Cas12i polypeptide.

Embodiment 17: A cell comprising the RNA guide, the nucleic acid, the vector, or the vector system of Embodiments 13-16 as described herein. In some examples, the cell is a eukaryotic cell, an animal cell, a mammalian cell, a human cell, a primary cell, a cell line, a stem cell, a neuron, or a T cell.

Embodiment 18: A kit comprising the RNA guide, the nucleic acid, the vector, or the vector system of Embodiments 13-16 as described herein.

Embodiment 19: A method of editing an STMN2 sequence, the method comprising contacting an STMN2 sequence with an RNA guide of Embodiment 13 as described herein. In some examples, the STMN2 sequence is in a cell.

In some examples, the RNA guide induces an indel (e.g., an insertion or deletion) in the STMN2 sequence.

Embodiment 20: A method of treating neurodegenerative diseases (e.g., amyotrophic lateral sclerosis (ALS) or frontotemporal dementia (FTD)), in a subject, the method comprising administering the RNA guide of Embodiment 13 as described herein to the subject.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty, ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the present disclosure in any way whatsoever. All publications cited herein are incorporated by reference for the purposes or subject matter referenced herein.

### EXAMPLES

The following examples are provided to further illustrate some embodiments of the present disclosure but are not intended to limit the scope of the present disclosure; it will be understood by their exemplary nature that other procedures, methodologies, or techniques known to those skilled in the art may alternatively be used.

### Example 1—Targeting of STMN2 Intron 1 by Variant Cas12i2

This Example describes indel assessment on multiple targets at the STMN2 gene in cells after transfection with plasmids coding for variant Cas12i2 (SEQ ID NO: 450) and RNA guides.

The variant Cas12i2 polypeptide was cloned into a plasmid comprising a CMV promoter. Fragments coding for RNA guides targeting the STMN2 intron 1 gene were cloned into a pUC19 backbone (New England Biolabs). The plasmids were then maxi-prepped and diluted. The crRNA, target, and PAM sequences are listed in Table 6.

TABLE 6
Mammalian targets and corresponding crRNAs.

| Target | SEQ | | SEQ | | PAM |
|---|---|---|---|---|---|
| identifier | ID NO | crRNA sequence | ID NO | Target sequence | sequence |
| 1 | 4505 | AGAAAUCCGUCUUUCAUU | 4563 | TGCCCCATCACTCTCTCT | TTC |
| or I1T1 | | GACGGUGCCCCAUCACUCU | | TA | |
| | | CUCUUA | | | |
| 2 | 4506 | AGAAAUCCGUCUUUCAUU | 4564 | ATTGGATTTTTAAAATTA | TTA |
| or I1T2 | | GACGGAUUGGAUUUUUAA | | TA | |
| | | AAUUAUA | | | |
| 3 | 4507 | AGAAAUCCGUCUUUCAUU | 4565 | GATTTTTAAAATTATATT | TTG |
| or I1T3 | | GACGGGAUUUUUAAAAUU | | CA | |
| | | AUAUUCA | | | |
| 4 | 4508 | AGAAAUCCGUCUUUCAUU | 4566 | TTAAAATTATATTCATAT | TTT |
| or I1T4 | | GACGGUUAAAAUUAUAUU | | TG | |
| | | CAUAUUG | | | |
| 5 | 4509 | AGAAAUCCGUCUUUCAUU | 4567 | TAAAATTATATTCATATT | TTT |
| or I1T5 | | GACGGUAAAAUUAUAUUC | | GC | |
| | | AUAUUGC | | | |
| 6 | 4510 | AGAAAUCCGUCUUUCAUU | 4568 | AAAATTATATTCATATTG | TTT |
| or I1T6 | | GACGGAAAAUUAUAUUCA | | CA | |
| | | UAUUGCA | | | |
| 7 | 4511 | AGAAAUCCGUCUUUCAUU | 4569 | AAATTATATTCATATTGC | TTA |
| or I1T7 | | GACGGAAAUUAUAUUCAU | | AG | |
| | | AUUGCAG | | | |
| 8 | 4512 | AGAAAUCCGUCUUUCAUU | 4570 | TATTCATATTGCAGGACT | TTA |
| or I1T8 | | GACGGUAUUCAUAUUGCA | | CG | |
| | | GGACUCG | | | |
| 9 | 4513 | AGAAAUCCGUCUUUCAUU | 4571 | ATATTGCAGGACTCGGC | TTC |
| or I1T9 | | GACGGAUAUUGCAGGACU | | AGA | |
| | | CGGCAGA | | | |
| 10 | 4514 | AGAAAUCCGUCUUUCAUU | 4572 | CAGGACTCGGCAGAAGA | TTG |
| or I1T10 | | GACGGCAGGACUCGGCAG | | CCT | |
| | | AAGACCU | | | |
| 11 | 4515 | AGAAAUCCGUCUUUCAUU | 4573 | GAGAGAAAGGTAGAAA | TTC |
| or I1T11 | | GACGGGAGAGAAAGGUAG | | ATAA | |
| | | AAAAUAA | | | |
| 12 | 4516 | AGAAAUCCGUCUUUCAUU | 4574 | GGCTCTCTGTGTGAGCA | TTT |
| or I1T12 | | GACGGGGCUCUCUGUGUG | | TGT | |
| | | AGCAUGU | | | |
| 13 | 4517 | AGAAAUCCGUCUUUCAUU | 4575 | GCTCTCTGTGTGAGCAT | TTG |
| or I1T13 | | GACGGGCUCUCUGUGUGA | | GTG | |
| | | GCAUGUG | | | |
| 14 | 4518 | AGAAAUCCGUCUUUCAUU | 4576 | TGGCACAGTTGACAAGG | TTG |
| or I1T14 | | GACGGUGGCACAGUUGAC | | ATG | |
| | | AAGGAUG | | | |
| 15 | 4519 | AGAAAUCCGUCUUUCAUU | 4577 | ACAAGGATGATAAATCA | TTG |
| or I1T15 | | GACGGACAAGGAUGAUAA | | ATA | |
| | | AUCAAUA | | | |
| 16 | 4520 | AGAAAUCCGUCUUUCAUU | 4578 | CTATCATTTATGAATAGC | TTA |
| or I1T16 | | GACGGCUAUCAUUUAUGA | | AA | |
| | | AUAGCAA | | | |
| 17 | 4521 | AGAAAUCCGUCUUUCAUU | 4579 | ATGAATAGCAATACTGA | TTT |
| or I1T17 | | GACGGAUGAAUAGCAAUA | | AGA | |
| | | CUGAAGA | | | |
| 18 | 4522 | AGAAAUCCGUCUUUCAUU | 4580 | TGAATAGCAATACTGAA | TTA |
| or I1T18 | | GACGGUGAAUAGCAAUAC | | GAA | |
| | | UGAAGAA | | | |
| 19 | 4523 | AGAAAUCCGUCUUUCAUU | 4581 | AAACAAAAGATTGCTGT | TTA |
| or I1T19 | | GACGGAAACAAAAGAUUG | | CTC | |
| | | CUGUCUC | | | |
| 20 | 4524 | AGAAAUCCGUCUUUCAUU | 4582 | CTGTCTCAATATATCTTA | TTG |
| or I1T20 | | GACGGCUGUCUCAAUAUA | | TA | |
| | | UCUUAUA | | | |
| 21 | 4525 | AGAAAUCCGUCUUUCAUU | 4583 | TATTTATTATTTACCAAA | TTA |
| or I1T21 | | GACGGUAUUUAUUAUUUA | | TT | |
| | | CCAAAUU | | | |
| 22 | 4526 | AGAAAUCCGUCUUUCAUU | 4584 | AGGAAGAAATACTCTTA | TTC |
| or I1T22 | | GACGGAGGAAGAAAUACU | | GAA | |
| | | CUUAGAA | | | |
| 23 | 4527 | AGAAAUCCGUCUUUCAUU | 4585 | GAATAATTTGGTAAATA | TTA |
| or I1T23 | | GACGGGAAUAAUUUGGUA | | ATA | |
| | | AAUAAUA | | | |
| 24 | 4528 | AGAAAUCCGUCUUUCAUU | 4586 | GGTAAATAATAAATATA | TTT |
| or I1T24 | | GACGGGGUAAAUAAUAAA | | AGA | |
| | | UAUAAGA | | | |
| 25 | 4529 | AGAAAUCCGUCUUUCAUU | 4587 | GTAAATAATAAATATAA | TTG |
| or I1T25 | | GACGGGUAAAUAAUAAAU | | GAT | |
| | | AUAAGAU | | | |
| 26 | 4530 | AGAAAUCCGUCUUUCAUU | 4588 | AGACAGCAATCTTTTGTT | TTG |
| or I1T26 | | GACGGAGACAGCAAUCUU | | TT | |
| | | UUGUUUU | | | |
| 27 | 4531 | AGAAAUCCGUCUUUCAUU | 4589 | TGTTTTAATTTCTTCAGT | TTT |
| or I1T27 | | GACGGUGUUUUAAUUUCU | | AT | |
| | | UCAGUAU | | | |
| 28 | 4532 | AGAAAUCCGUCUUUCAUU | 4590 | GTTTTAATTTCTTCAGTA | TTT |
| or I1T28 | | GACGGGUUUUAAUUUCUU | | TT | |
| | | CAGUAUU | | | |
| 29 | 4533 | AGAAAUCCGUCUUUCAUU | 4591 | TTTTAATTTCTTCAGTAT | TTG |
| or I1T29 | | GACGGUUUUAAUUUCUUC | | TG | |
| | | AGUAUUG | | | |
| 30 | 4534 | AGAAAUCCGUCUUUCAUU | 4592 | TAATTTCTTCAGTATTGC | TTT |
| or I1T30 | | GACGGUAAUUUCUUCAGU | | TA | |
| | | AUUGCUA | | | |
| 31 | 4535 | AGAAAUCCGUCUUUCAUU | 4593 | AATTTCTTCAGTATTGCT | TTT |
| or I1T31 | | GACGGAAUUUCUUCAGUA | | AT | |
| | | UUGCUAU | | | |
| 32 | 4536 | AGAAAUCCGUCUUUCAUU | 4594 | ATTTCTTCAGTATTGCTA | TTA |
| or I1T32 | | GACGGAUUUCUUCAGUAU | | TT | |
| | | UGCUAUU | | | |
| 33 | 4537 | AGAAAUCCGUCUUUCAUU | 4595 | CTTCAGTATTGCTATTCA | TTT |
| or I1T33 | | GACGGCUUCAGUAUUGCU | | TA | |
| | | AUUCAUA | | | |
| 34 | 4538 | AGAAAUCCGUCUUUCAUU | 4596 | TTCAGTATTGCTATTCAT | TTC |
| or I1T34 | | GACGGUUCAGUAUUGCUA | | AA | |
| | | UUCAUAA | | | |
| 35 | 4539 | AGAAAUCCGUCUUUCAUU | 4597 | AGTATTGCTATTCATAA | TTC |
| or I1T35 | | GACGGAGUAUUGCUAUUC | | ATG | |
| | | AUAAAUG | | | |
| 36 | 4540 | AGAAAUCCGUCUUUCAUU | 4598 | CTATTCATAAATGATAG | TTG |
| or I1T36 | | GACGGCUAUUCAUAAAUG | | TAA | |
| | | AUAGUAA | | | |
| 37 | 4541 | AGAAAUCCGUCUUUCAUU | 4599 | ATAAATGATAGTAAGCT | TTC |
| or I1T37 | | GACGGAUAAAUGAUAGUA | | TGC | |
| | | AGCUUGC | | | |
| 38 | 4542 | AGAAAUCCGUCUUUCAUU | 4600 | CATTATTGATTTATCATC | TTG |
| or I1T38 | | GACGGCAUUAUUGAUUUA | | CT | |
| | | UCAUCCU | | | |
| 39 | 4543 | AGAAAUCCGUCUUUCAUU | 4601 | TTGATTTATCATCCTTGT | TTA |
| or I1T39 | | GACGGUUGAUUUAUCAUC | | CA | |
| | | CUUGUCA | | | |
| 40 | 4544 | AGAAAUCCGUCUUUCAUU | 4602 | ATTTATCATCCTTGTCAA | TTG |
| or I1T40 | | GACGGAUUUAUCAUCCUU | | CT | |
| | | GUCAACU | | | |
| 41 | 4545 | AGAAAUCCGUCUUUCAUU | 4603 | ATCATCCTTGTCAACTGT | TTT |
| or I1T41 | | GACGGAUCAUCCUUGUCA | | GC | |
| | | ACUGUGC | | | |
| 42 | 4546 | AGAAAUCCGUCUUUCAUU | 4604 | TCATCCTTGTCAACTGTG | TTA |
| or I1T42 | | GACGGUCAUCCUUGUCAA | | CC | |
| | | CUGUGCC | | | |
| 43 | 4547 | AGAAAUCCGUCUUUCAUU | 4605 | TCAACTGTGCCACAAGC | TTG |
| or I1T43 | | GACGGUCAACUGUGCCAC | | CGC | |
| | | AAGCCGC | | | |
| 44 | 4548 | AGAAAUCCGUCUUUCAUU | 4606 | ACATTCATTTCTTCTTAG | TTC |
| or I1T44 | | GACGGACAUUCAUUUCUU | | GC | |
| | | CUUAGGC | | | |
| 45 | 4549 | AGAAAUCCGUCUUUCAUU | 4607 | ATTTCTTCTTAGGCAGGC | TTC |
| or I1T45 | | GACGGAUUUCUUCUUAGG | | TG | |
| | | CAGGCUG | | | |
| 46 | 4550 | AGAAAUCCGUCUUUCAUU | 4608 | CTTCTTAGGCAGGCTGTC | TTT |
| or I1T46 | | GACGGCUUCUUAGGCAGG | | TG | |
| | | CUGUCUG | | | |
| 47 | 4551 | AGAAAUCCGUCUUUCAUU | 4609 | TTCTTAGGCAGGCTGTCT | TTC |
| or I1T47 | | GACGGUUCUUAGGCAGGC | | GT | |
| | | UGUCUGU | | | |
| 48 | 4552 | AGAAAUCCGUCUUUCAUU | 4610 | TTAGGCAGGCTGTCTGT | TTC |
| or I1T48 | | GACGGUUAGGCAGGCUGU | | CTC | |
| | | CUGUCUC | | | |
| 49 | 4553 | AGAAAUCCGUCUUUCAUU | 4611 | GGCAGGCTGTCTGTCTCT | TTA |
| or I1T49 | | GACGGGGCAGGCUGUCUG | | CT | |
| | | UCUCUCU | | | |
| 50 | 4554 | AGAAAUCCGUCUUUCAUU | 4612 | TTATTTTCTACCTTTCTC | TTC |
| or I1T50 | | GACGGUUAUUUUCUACCU | | TC | |
| | | UUCUCUC | | | |
| 51 | 4555 | AGAAAUCCGUCUUUCAUU | 4613 | TTTTCTACCTTTCTCTCG | TTA |
| or I1T51 | | GACGGUUUUCUACCUUUC | | AA | |
| | | UCUCGAA | | | |
| 52 | 4556 | AGAAAUCCGUCUUUCAUU | 4614 | TCTACCTTTCTCTCGAAG | TTT |
| or I1T52 | | GACGGUCUACCUUUCUCUC | | GT | |
| | | GAAGGU | | | |
| 53 | 4557 | AGAAAUCCGUCUUUCAUU | 4615 | CTACCTTTCTCTCGAAGG | TTT |
| or I1T53 | | GACGGCUACCUUUCUCUCG | | TC | |
| | | AAGGUC | | | |
| 54 | 4558 | AGAAAUCCGUCUUUCAUU | 4616 | TACCTTTCTCTCGAAGGT | TTC |
| or I1T54 | | GACGGUACCUUUCUCUCG | | CT | |
| | | AAGGUCU | | | |
| 55 | 4559 | AGAAAUCCGUCUUUCAUU | 4617 | CTCTCGAAGGTCTTCTGC | TTT |
| or I1T55 | | GACGGCUCUCGAAGGUCU | | CG | |
| | | UCUGCCG | | | |
| 56 | 4560 | AGAAAUCCGUCUUUCAUU | 4618 | TCTCGAAGGTCTTCTGCC | TTC |
| or I1T56 | | GACGGUCUCGAAGGUCUU | | GA | |
| | | CUGCCGA | | | |
| 57 | 4561 | AGAAAUCCGUCUUUCAUU | 4619 | TGCCGAGTCCTGCAATA | TTC |
| or I1T57 | | GACGGUGCCGAGUCCUGC | | TGA | |
| | | AAUAUGA | | | |
| 58 | 4562 | AGAAAUCCGUCUUUCAUU | 4620 | TAAAAATCCAATTAAGA | TTT |
| or I1T58 | | GACGGUAAAAAUCCAAUU | | GAG | |
| | | AAGAGAG | | | |

Approximately 16 hours prior to transfection, 25,000 HEK293T cells in DMEM/10% FBS+Pen/Strep (D10 media) were plated into each well of a 96-well plate. On the day of transfection, the cells were 70-90% confluent. For each well to be transfected, a mixture of LIPOFECTAMINE® 2000 transfection reagent (ThermoFisher) and Opti-MEM® reduced serum medium (ThermoFisher) was prepared and incubated at room temperature for 5 minutes (Solution 1). After incubation, the LIPOFECTAMINE® 2000: Opti-MEM® (transfection reagent (ThermoFisher):reduced serum medium (ThermoFisher)) mixture was added to a separate mixture containing nuclease plasmid, RNA guide plasmid, and Opti-MEM® reduced serum medium (ThermoFisher) (Solution 2). In the case of negative controls, the RNA guide plasmid was not included in Solution 2. Solution 1 and 2 were pipette mixed 8 times, then incubated at room temperature for 25 minutes. Following incubation, the Solution 1 and 2 mixture was added dropwise to each well of a 96-well plate containing the cells. 72 hours post transfection, cells were trypsinized by adding TRYPLE™ (recombinant cell-dissociation enzymes; ThermoFisher) to the center of each well and incubating at 37° C. for approximately 5 minutes. D10 media was then added to each well and mixed to resuspend cells. The resuspended cells were centrifuged at 500×g for 10 minutes to obtain a pellet, and the supernatant was discarded. QUICKEXTRACT™ (DNA extraction solution; Lucigen) extraction reagent was added to each well to lyse pelleted cells. Cells were incubated at 65° C. for 15 minutes, 68° C. for 15 minutes, and 98° C. for 10 minutes.

Samples for NGS were prepared by two rounds of PCR. The first round (PCR1) was used to amplify specific genomic regions depending on the target. Round 2 PCR (PCR2) was performed to add Illumina adapters and indices. Reactions were then pooled and purified by column purification. Sequencing runs were done with a 300 Cycle NEXTSEQ™ (Illumina) 500/550 High Output v2.5 Kit.

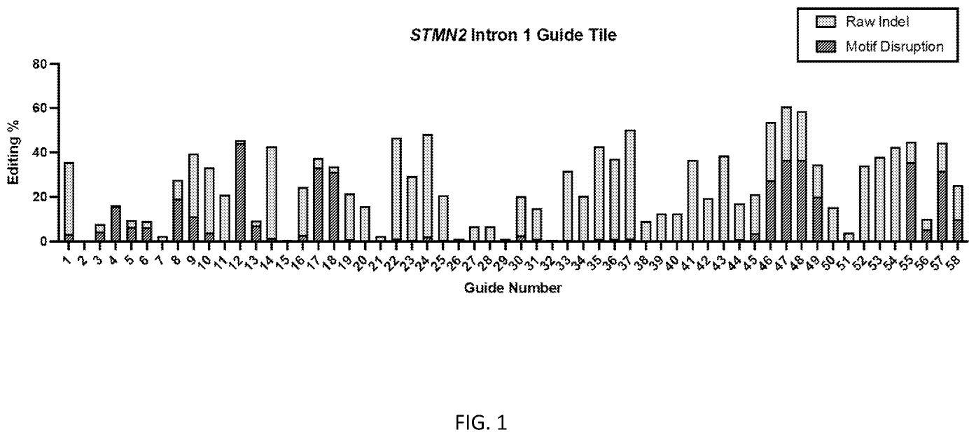

As shown in FIG. 1, RNA guides 1, 3, 4, 5, 6, 8, 9, 10, 11, 12, 13, 14, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, and 58 all resulted in measurable indel activity, defined as >1% and >0.2% above the background rate for the no-RNA guide control.

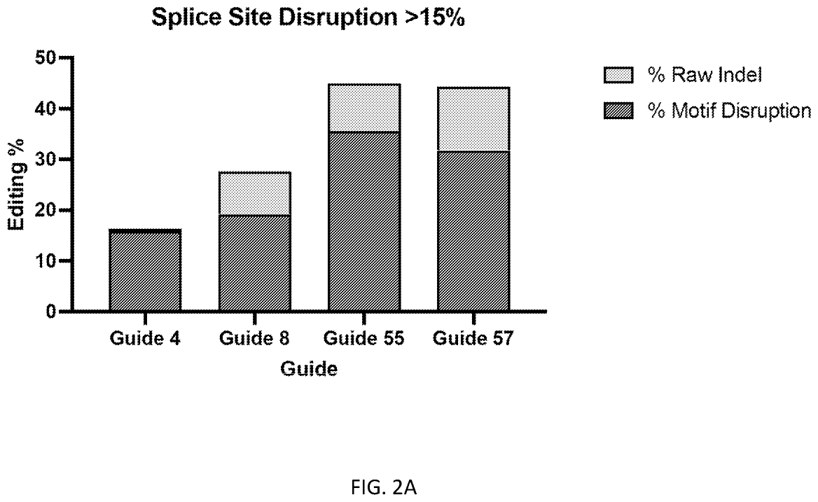

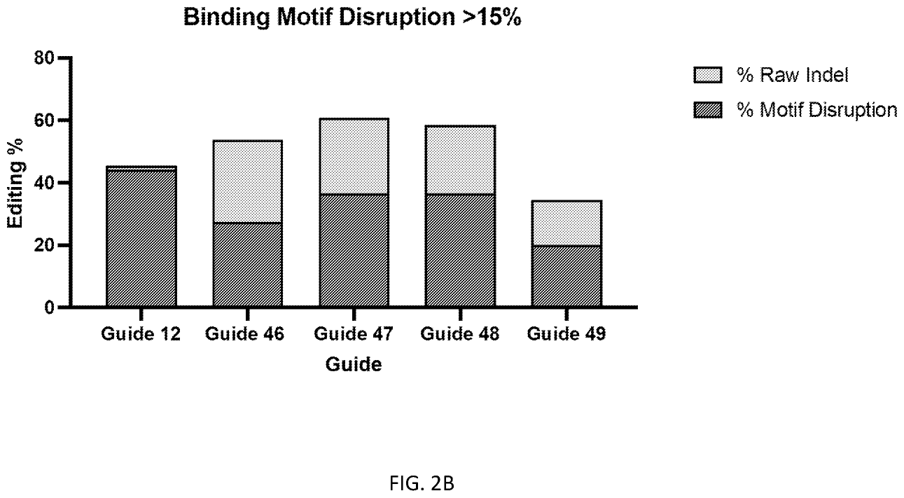

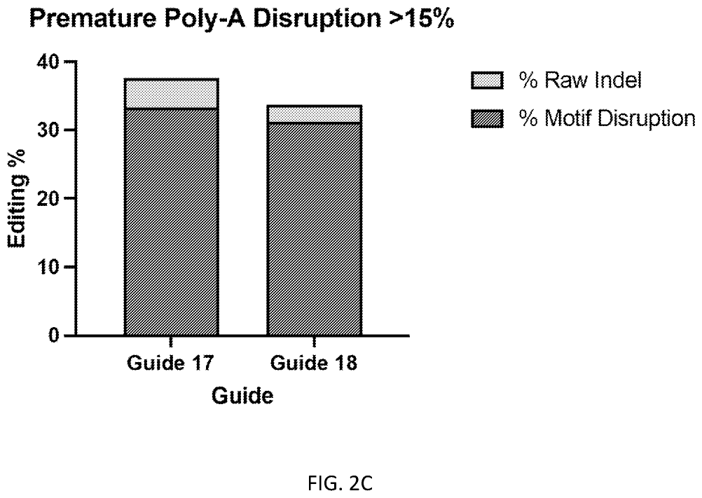

RNA guides 4, 8, 55, and 57 resulted in >15% disruption of the cryptic splice site in intron 1 (FIG. 2A), where disruption is defined as an insertion or deletion at one or more bases of the cryptic splice site. 97% of the indels generated by RNA guide 4 resulted in disruption of the cryptic splice site in intron 1, where disruption is defined as an insertion or deletion at one or more bases of the cryptic splice site.

RNA guides 12, 46, 47, 48, and 49 resulted in >15% disruption of at least one of 3 TDP-43 binding motifs in intron 1 (FIG. 2B), where disruption is defined as an insertion or deletion at one or more bases of a TDP-43 binding motif. 97% of the indels generated by RNA guide 12 resulted in disruption of at least one of 3 TDP-43 binding motifs in intron 1, where disruption is defined as an insertion or deletion at one or more bases of a TDP-43 binding motif.

RNA guides 17 and 18 resulted in >15% disruption of the premature polyadenylation signal in intron 1 (FIG. 2C), where disruption is defined as an insertion or deletion at one or more bases of the polyadenylation signal. 88% of the indels generated by RNA guide 17 resulted in disruption of the premature polyadenylation signal at intron 1, where disruption is defined as an insertion or deletion at one or more bases of the premature polyadenylation site. 93% of the indels generated by RNA guide 18 resulted in disruption of the premature polyadenylation signal at intron 1, where disruption is defined as an insertion or deletion at one or more bases of the premature polyadenylation site.

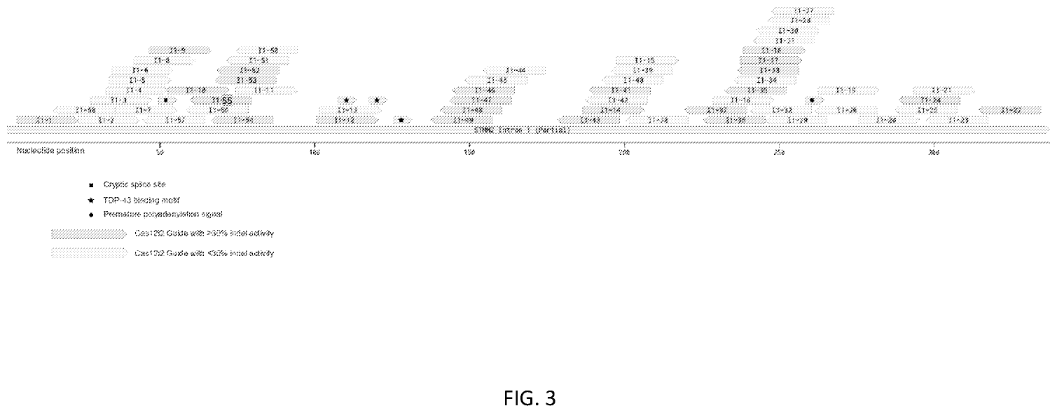

FIG. 3 depicts the positions where each of the RNA guides binds intron 1 of STMN2 relative to the positions of the cryptic splice site, the TDP-43 binding motifs, and the premature polyadenylation signal. The darker grey reflects RNA guides demonstrating indels in greater than 30% of NGS reads, and the lighter grey reflects RNA guides demonstrating indels in less than 30% of NGS read. This Example thus shows that Cas12i2 guides edited intron 1 of STMN2 and were able to disrupt the cryptic splice site, TDP-43 binding motifs, and premature polyadenylation signals.

### Example 2—Targeting of STMN2 Intron 1 by Variant Cas12i2 in SH-SY % Y Cells

This Example describes indel assessment on multiple targets at the STMN2 gene in a neuroblastoma cell line after transfection with plasmids coding for variant Cas12i2 (SEQ ID NO: 450) and RNA guides targeting the cryptic splice site of intron 1.

The variant Cas12i2 polypeptide and RNA guides 4, 5, 8, 9, 55, 56, 57, and 58 of Table 6 were cloned, purified, and diluted as described in Example 1. Approximately 16 hours prior to transfection. 25.000 SH-SY5Y cells in EMEM:F12/10% FBS+Pen/Strep (EF12-10 media) were plated into each well of a 96-well plate. On the day of transfection, the cells were 70-90% confluent. For each well to be transfected, a mixture of LIPOFECTAMINE® 2000 transfection reagent (ThermoFisher) and Opti-MEM® reduced serum medium (ThermoFisher) was prepared and incubated at room temperature for 5 minutes (Solution 1). After incubation, the LIPOFECTAMINE® 2000: Opti-MEM® (transfection reagent (ThermoFisher):reduced serum medium (ThermoFisher)) mixture was added to a separate mixture containing nuclease plasmid. RNA guide plasmid, and Opti-MEM® reduced serum medium (ThermoFisher) (Solution 2). In the case of negative controls, the RNA guide plasmid was not included in Solution 2. Solution 1 and 2 were pipette mixed 8 times, then incubated at room temperature for 25 minutes. Following incubation, the Solution 1 and 2 mixture was added dropwise to each well of a 96-well plate containing the cells. 72 hours post transfection, cells were trypsinized by adding TRYPLE™ (recombinant cell-dissociation enzymes; ThermoFisher) to the center of each well and incubating at 37° C. for approximately 5 minutes. EF12-10 media was then added to each well and mixed to resuspend cells. The resuspended cells were centrifuged at 500×g for 10 minutes to obtain a pellet, and the supernatant was discarded. QUICKEXTRACT™ (DNA extraction solution; Lucigen) extraction reagent was added to each well to lyse pelleted cells. Cells were incubated at 65° C. for 15 minutes. 68° C. for 15 minutes, and 98° C. for 10 minutes.

Samples for NGS were prepared by two rounds of PCR. The first round (PCR1) was used to amplify specific genomic regions depending on the target. Round 2 PCR (PCR2) was performed to add Illumina adapters and indices. Reactions were then pooled and purified by column purification. Sequencing runs were done with a 300 Cycle NEXTSEQ™ (Illumina) 500/550 High Output v2.5 Kit.

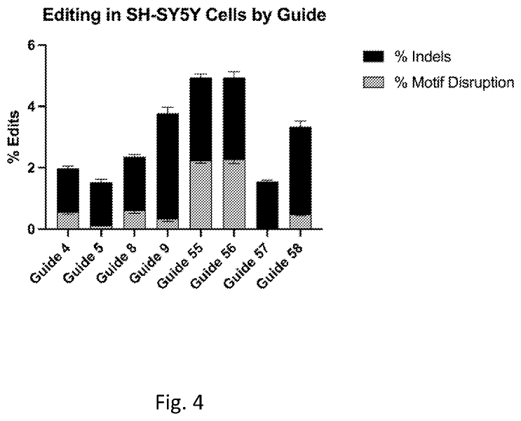

FIG. 4 shows indel activity of the tested RNA guides in SH-SY5Y cells. Guide 4 showed 0.56% splice site motif disruption and 2.0% overall editing; greater than 25% of total edits disrupted the splice site. Guide 5 showed 0.12% splice site motif disruption and 1.5% overall editing; less than 10% of total edits disrupted the splice site. Guide 8 showed 0.62% splice site motif disruption and 2.4% overall editing; greater than 25% of total edits disrupted the splice site. Guide 9 showed 0.34% splice site motif disruption and 3.8% overall editing; less than 10% of total edits disrupted the splice site. Guide 55 showed 2.2% splice site motif disruption and 4.9% overall editing; greater than 40% of total edits disrupted the splice site. Guide 56 showed 2.3% splice site motif disruption and 4.9% overall editing; greater than 45% of total edits disrupted the splice site. Guide 57 showed 0% splice site motif disruption and 1.6% overall editing. Guide 58 showed 0.49% splice site motif disruption and 3.3% overall editing; greater than 10% of total edits disrupted the splice site.

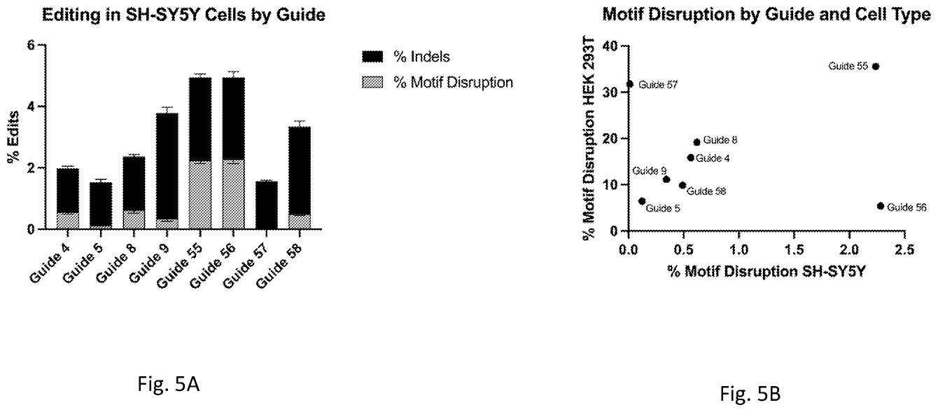

FIG. 5A is a plot comparing indel activity (% indels) demonstrated in HEK293T cells and SH-SY5Y cells from Example 1 and Example 2, respectively. FIG. 5B is a plot comparing splice site motif disruption demonstrated in HEK293T cells and SH-SY5Y cells from Example 1 and Example 2, respectively. As shown in FIG. 5A, Guide 55 and Guide 9 demonstrated the highest % indels across the two cell types. Guide 56 demonstrated the highest % indels in SH-SY5Y cells but low % indels in HEK293T cells. Guide 55 resulted in the highest splice site motif disruption in the two cell types as well (FIG. 5B).

This Example thus shows that the cryptic splice site of intron 1 of STMN2 is capable of being targeted by Cas12i2 and multiple RNA guides in multiple cell types.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the present disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either.” “one of,” “only one of.” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A gene editing system for genetic editing of a stathmin 2 (STMN2) gene, comprising
(i) a Cas12i2 polypeptide or a first nucleic acid encoding the Cas12i2 polypeptide, wherein the Cas12i2 polypeptide comprises an amino acid sequence at least 95% identical to SEQ ID NO: 448 and comprises one or more mutations relative to SEQ ID NO: 448; and
(ii) an RNA guide or a second nucleic acid encoding the RNA guide, wherein the RNA guide comprises a spacer sequence specific to a target sequence within an STMN2 gene, the target sequence being adjacent to a protospacer adjacent motif (PAM) comprising the motif of 5′-TTN-3′, which is located 5′ to the target sequence.

2. The gene editing system of claim 1, wherein the one or more mutations in the Cas12i2 polypeptide are at positions D581, G624, F626, P868, I926, V1030, E1035, and/or S1046 of SEQ ID NO: 448.

3. The gene editing system of claim 1, wherein the one or more mutations are amino acid substitutions, which optionally is D581R, G624R, F626R, P868T, 1926R, V1030G, E1035R, S1046G, or a combination thereof.

4. The gene editing system of claim 3, wherein the Cas12i2 polypeptide comprises:
(i) mutations at positions D581, D911, I926, and V1030, which optionally are amino acid substitutions of D581R, D911R, 1926R, and V1030G;
(ii) mutations at positions D581, I926, and V1030, which optionally are amino acid substitutions of D581R, 1926R, and V1030G;
(iii) mutations at positions D581, I926, V1030, and S1046, which optionally are amino acid substitutions of D581R, 1926R, V1030G, and S1046G;
(iv) mutations at positions D581, G624, F626, I926, V1030, E1035, and S1046, which optionally are amino acid substitutions of D581R, G624R, F626R, 1926R, V1030G, E1035R, and S1046G; or
(v) mutations at positions D581, G624, F626, P868, I926, V1030, E1035, and S1046, which optionally are amino acid substitutions of D581R, G624R, F626R, P868T, 1926R, V1030G, E1035R, and S1046G.

5. The gene editing system of claim 1, wherein the Cas12i2 polypeptide comprises the amino acid sequence of SEQ ID NO: 449, 450, 451, 452, or 453, optionally wherein the Cas12i2 polypeptide comprises the amino acid sequence of SEQ ID NO: 450 or 453.

6. The gene editing system of claim 1, which comprises the first nucleic acid encoding the Cas12i2 polypeptide.

7. The gene editing system of claim 6, wherein the first nucleic acid is a messenger RNA (mRNA).

8. The gene editing system of claim 6, wherein the first nucleic acid is included in a viral vector, which optionally is an adeno-associated viral (AAV) vector.

9. The gene editing system of claim 1, wherein the target sequence is within exon 1, exon 2, exon 3, exon 4, exon 5, exon 6, exon 7, or an intron of the STMN2 gene.

10. The gene editing system of claim 1, wherein the RNA guide comprises the sequence of any one of SEQ ID NOs: 4508, 4512, 4559, and 4561, or the second nucleic acid encodes an RNA guide comprising any one of SEQ ID NOs: 4508, 4512, 4559, and 4561.

11. The gene editing system of claim 1, wherein the RNA guide comprises the sequence of any one of SEQ ID NOs: 4505, 4506, 4507, 4508, 4509, 4510, 4511, 4512, 4513, 4514, 4515, 4554, 4555, 4556, 4557, 4558, 4559, 4560, 4561, and 4562, or the second nucleic acid encodes an RNA guide comprising any one of SEQ ID NOs: 4505, 4506, 4507, 4508, 4509, 4510, 4511, 4512, 4513, 4514, 4515, 4554, 4555, 4556, 4557, 4558, 4559, 4560, 4561, and 4562.

12. The gene editing system of claim 1, wherein the spacer sequence is 20-30-nucleotides in length, optionally wherein the spacer sequence is 20-nucleotides in length.

13. The gene editing system of claim 1, wherein the RNA guide comprises the spacer sequence and a direct repeat sequence.

14. The gene editing system of claim 13, wherein the direct repeat sequence is 23-36-nucleotides in length.

15. The gene editing system of claim 14, wherein the direct repeat sequence is at least 90% identical to any one of SEQ ID NOs: 1-10 or a fragment thereof that is at least 23-nucleotides in length.

16. The gene editing system of claim 15, wherein the direct repeat sequence is any one of SEQ ID NOs: 1-10, or a fragment thereof that is at least 23-nucleotides in length.

17. The gene editing system of claim 16, wherein the direct repeat sequence is 5′-AGAAAUCCGUCUUUCAUUGACGG-3′ (SEQ ID NO: 10).

18. The gene editing system of claim 1, wherein the system comprises the second nucleic acid encoding the RNA guide.

19. The gene editing system of claim 18, wherein the second nucleic acid encoding the RNA guide is located in a viral vector.

20. The gene editing system of claim 8, wherein the viral vector comprises the both the first nucleic acid encoding the Cas12i2 polypeptide and the second nucleic acid encoding the RNA guide.

21. The gene editing system of claim 1, wherein the system comprises the first nucleic acid encoding the Cas12i2 polypeptide, which is located on a first vector, and wherein the system comprises the second nucleic acid encoding the RNA guide, which is located on a second vector.

22. The gene editing system of claim 21, wherein the first and second vector are the same vector.

23. The gene editing system of claim 1, wherein the system comprises one or more lipid nanoparticles (LNPs), which encompass (i), (ii), or both.

24. The gene editing system of claim 23, wherein the system comprises the LNP, which encompass (i), and wherein the system comprises a viral vector comprising the second nucleic acid encoding the RNA guide; optionally wherein the viral vector is an AAV vector.

25. The gene editing system of claim 23, wherein the system comprises the LNP, which encompass (ii), and wherein the system comprises a viral vector comprising the first nucleic acid encoding Cas12i2 polypeptide; optionally wherein the viral vector is an AAV vector.

26. A gene editing system for genetic editing of a stathmin 2 (STMN2) gene, comprising
(i) a Cas12i polypeptide or a first nucleic acid encoding the Cas12i polypeptide, optionally wherein the Cas12i polypeptide is a Cas12i2 polypeptide; and
(ii) an RNA guide or a second nucleic acid encoding the RNA guide, wherein the RNA guide comprises a spacer sequence specific to a target sequence within exon 1, exon 2, exon 3, exon 4, exon 5, exon 6, exon 7, or an intron of a STMN2 gene, the target sequence being adjacent to a protospacer adjacent motif (PAM) comprising the motif of 5′-TTN-3′, which is located 5′ to the target sequence.

27. The gene editing system of 26, wherein the RNA guide comprises the sequence of any one of SEQ ID NOs: 4508, 4512, 4559, and 4561, or the second nucleic acid encodes an RNA guide comprising any one of SEQ ID NOs: 4508, 4512, 4559, and 4561.

28. The gene editing system of claim 26, wherein the RNA guide comprises the sequence of any one of SEQ ID NOs: 4505, 4506, 4507, 4508, 4509, 4510, 4511, 4512, 4513, 4514, 4515, 4554, 4555, 4556, 4557, 4558, 4559, 4560, 4561, and 4562 or the second nucleic acid encodes an RNA guide comprising any one of SEQ ID NOs: 4505, 4506, 4507, 4508, 4509, 4510, 4511, 4512, 4513, 4514, 4515, 4554, 4555, 4556, 4557, 4558, 4559, 4560, 4561, and 4562.

29. The gene editing system of claim 26, which comprises the first nucleic acid encoding the Cas12i polypeptide.

30. The gene editing system of claim 29, wherein the first nucleic acid is a messenger RNA (mRNA).

31. The gene editing system of claim 29, wherein the first nucleic acid is included in a viral vector, which optionally is an adeno-associated viral (AAV) vector.

32. The gene editing system of claim 26, wherein the spacer sequence is 20-30-nucleotides in length, optionally wherein the spacer sequence is 20-nucleotides in length.

33. The gene editing system of claim 26, wherein the RNA guide comprises the spacer sequence and a direct repeat sequence.

34. The gene editing system of claim 33, wherein the direct repeat sequence is 23-36-nucleotides in length.

35. The gene editing system of claim 34, wherein the direct repeat sequence is at least 90% identical to any one of SEQ ID NOs: 1-10 or a fragment thereof that is at least 23-nucleotides in length.

36. The gene editing system of claim 35, wherein the direct repeat sequence is any one of SEQ ID NOs: 1-10, or a fragment thereof that is at least 23-nucleotides in length.

37. The gene editing system of claim 36, wherein the direct repeat sequence is 5′-AGAAAUCCGUCUUUCAUUGACGG-3′ (SEQ ID NO: 10).

38. The gene editing system of claim 26, wherein the system comprises the second nucleic acid encoding the RNA guide.

39. The gene editing system of claim 38, wherein the second nucleic acid encoding the RNA guide is located in a viral vector.

40. The gene editing system of claim 31, wherein the viral vector comprises the both the first nucleic acid encoding the Cas12i polypeptide and the second nucleic acid encoding the RNA guide.

41. The gene editing system of claim 26, wherein the system comprises the first nucleic acid encoding the Cas12i polypeptide, which is located on a first vector, and wherein the system comprises the second nucleic acid encoding the RNA guide, which is located on a second vector.

42. The gene editing system of claim 26, wherein the system comprises one or more lipid nanoparticles (LNPs), which encompass (i), (ii), or both.

43. The gene editing system of claim 42, wherein the system comprises the LNP, which encompass (i), and wherein the system comprises a viral vector comprising the second nucleic acid encoding the RNA guide; optionally wherein the viral vector is an AAV vector.

44. The gene editing system of claim 42, wherein the system comprises the LNP, which encompass (ii), and wherein the system comprises a viral vector comprising the first nucleic acid encoding Cas12i polypeptide; optionally wherein the viral vector is an AAV vector.

45. A pharmaceutical composition comprising the gene editing system set forth in claim 1.

46. A kit comprising the elements (i) and (ii) of the gene editing system set forth in claim 1.

47. A method for editing a stathmin 2 (STMN2) gene in a cell, the method comprising contacting a host cell with the gene editing system for editing the STMN2 gene set forth in claim 1 to genetically edit the STMN2 gene in the host cell.

48. The method of claim 47, wherein the host cell is cultured in vitro.

49. The method of claim 47, wherein contacting step is performed by administering the gene editing system for editing the STMN2 gene to a subject comprising the host cell.

50. A cell comprising a disrupted stathmin 2 (STMN2) gene, wherein the cell optionally is produced by contacting a host cell with the gene editing system of claim 1 to genetically edit the STMN2 gene in the host cell, thereby disrupting the STMN2 gene.

51. A method for treating neurodegenerative diseases in a subject, comprising administering to a subject in need thereof a gene editing system for editing a stathmin 2 (STMN2) gene set forth in claim 1 or a cell comprising a disrupted STMN2 gene, wherein the cell optionally is produced by contacting a host cell with the gene editing system set forth in claim 1.

52. The method of claim 51, wherein the subject is a human patient having the neurodegenerative disease, which optionally is amyotrophic lateral sclerosis (ALS).

53. The method of claim 51, wherein the subject is a human patient having the neurodegenerative disease, which optionally is frontotemporal dementia (FTD).

54. An RNA guide, comprising (i) a spacer sequence that is specific to a target sequence in a stathmin 2 (STMN2) gene, wherein the target sequence is adjacent to a protospacer adjacent motif (PAM) comprising the motif of 5′-TTN-3′, which is located 5′ to the target sequence; and (ii) a direct repeat sequence.

55. The RNA guide of claim 54, wherein the spacer sequence is 20-30-nucleotides in length, optionally 20-nucleotides in length.

56. The RNA guide of claim 54, wherein the direct repeat sequence is 23-36-nucleotides in length, optionally 23-nucleotides in length.

57. The RNA guide of claim 54, wherein the target sequence is within exon 1, exon 2, exon 3, exon 4, exon 5, exon 6, exon 7, or an intron of the STMN2 gene.

58. The RNA guide of claim 54, wherein the RNA guide comprises the sequence of any one of SEQ ID NOs: 4508, 4512, 4559, and 4561.

59. The RNA guide of claim 54, wherein the RNA guide comprises the sequence of any one of SEQ ID NOs: 4505, 4506, 4507, 4508, 4509, 4510, 4511, 4512, 4513, 4514, 4515, 4554, 4555, 4556, 4557, 4558, 4559, 4560, 4561, and 4562.

60. The RNA guide of claim 54, wherein the direct repeat sequence is at least 90% identical to any one of SEQ ID NOs: 1-10 or a fragment thereof that is at least 23-nucleotides in length.

61. The RNA guide of claim 60, wherein the direct repeat sequence is any one of SEQ ID NOs: 1-10, or a fragment thereof that is at least 23-nucleotides in length.

62. The RNA guide of claim 61, wherein the direct repeat sequence is 5′-AGAAAUCCGUCUUUCAUUGACGG-3′ (SEQ ID NO: 10).
